(19) United States
(12) Patent Application Publication
(10) Pub. No.: US 2025/0370335 A1
(43) Pub. Date: Dec. 4, 2025

(54) SULFONIUM SALT MONOMER, POLYMER, CHEMICALLY AMPLIFIED RESIST COMPOSITION, AND PATTERN FORMING PROCESS

(71) Applicant: SHIN-ETSU CHEMICAL CO., LTD., Tokyo (JP)

(72) Inventors: Masahiro Fukushima, Joetsu-shi (JP); Daisuke Kori, Joetsu-shi (JP)

(73) Assignee: SHIN-ETSU CHEMICAL CO., LTD., Tokyo (JP)

(21) Appl. No.: 19/217,485

(22) Filed: May 23, 2025

(30) Foreign Application Priority Data
May 30, 2024 (JP) 2024-087965

(51) Int. Cl. G03F 7/004; C07C 381/12; C08F 20/10; G03F 7/00; G03F 7/039

(52) U.S. Cl. CPC G03F 7/0045; C07C 381/12; C08F 20/10; G03F 7/0046; G03F 7/0397; G03F 7/70033

(57) ABSTRACT

The sulfonium salt monomer used for a chemically amplified resist composition has excellent solvent solubility, high sensitivity, and high contrast, and excellent lithography performance such as exposure latitude (EL), LWR, CDU, and depth of focus (DOF), particularly in photolithography using high-energy radiation such as KrF excimer laser light, ArF excimer laser light, an electron beam (EB), and EUV. The sulfonium salt monomer having the formula (A).

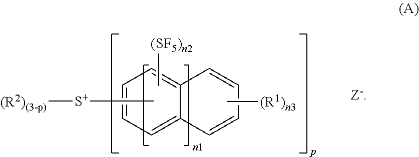

### CROSS-REFERENCE TO RELATED APPLICATION

This non-provisional application claims priority under 35 U.S.C. § 119(a) on Patent Application No. 2024-087965 filed in Japan on May 30, 2024, the entire contents of which are hereby incorporated by reference.

### TECHNICAL FIELD

This invention relates to a sulfonium salt monomer, a polymer, a chemically amplified resist composition, and a pattern forming process.

### BACKGROUND ART

To meet the demand for higher integration density and operating speed of LSIs, the effort to reduce the pattern rule is in rapid progress. The wide-spreading flash memory market and the demand for increased storage capacities drive forward the miniaturization technology. As the most advanced miniaturization technology, mass production of 65-nm node devices by ArF lithography has been implemented, and preparation for mass production of 45-nm node devices by next generation ArF immersion lithography is in progress. The candidates for the next generation 32-nm node devices include ultra-high NA lens immersion lithography using a liquid having a higher refractive index than water in combination with a high refractive index lens and a high refractive index resist film, extreme ultraviolet (EUV) lithography of wavelength 13.5 nm, and double patterning version of the ArF lithography, on which active research efforts have been made.

As the pattern feature size is reduced, approaching to the diffraction limit of light, light contrast lowers. In the case of positive resist film, a lowering of light contrast leads to reductions of resolution and focus margin of hole and trench patterns.

As the pattern feature size is reduced, the line width roughness (LWR) of line patterns and the critical dimension uniformity (CDU) of hole patterns are regarded significant. It is pointed out that these factors are affected by the segregation or agglomeration of a base polymer and an acid generator and the diffusion of an acid. There is a tendency that as the resist film becomes thinner, LWR becomes greater. A film thickness reduction to comply with the progress of size reduction causes a degradation of LWR, which becomes a serious problem.

The resist composition for EUV lithography need to achieve high sensitivity, high resolution, and low LWR at the same time. If the acid diffusion distance is shortened, the LWR becomes smaller, but the sensitivity is lowered. For example, as the post exposure bake (PEB) temperature is lowered, the LWR becomes smaller, but the sensitivity is lowered. The LWR becomes smaller even if the amount of the quencher added is increased, but the sensitivity is lowered. It is necessary to overcome the trade-off relationship between sensitivity and LWR.

In order to prevent acid diffusion, Patent Document 1 discloses a resist compound containing a repeat unit derived from an onium salt of sulfonic acid having a polymerizable unsaturated bond. Such a so-called polymer-bound acid generator is characterized by a very short distance of acid diffusion because a polymeric sulfonic acid is generated upon exposure. The sensitivity can also be enhanced by increasing the proportion of the acid generator. In the case of an addition type acid generators, as the amount of the acid generator added is increased, the sensitivity is increased, but in this case, the acid diffusion distance is also increased. Since the acid diffuses non-uniformly, LWR or CDU deteriorates as the acid diffusion increases. With respect to the balance of sensitivity, LWR, and CDU, the polymeric acid generator is regarded as having a high capability.

Since an iodine atom is highly absorptive to EUV with a wavelength of 13.5 nm, an effect of generating secondary electrons from the iodine atom upon exposure has been verified, and is noteworthy in EUV lithography. Patent Document 2 describes a photoacid generator having an iodized anion, and Patent Document 3 describes a photoacid generator having an iodized anion and containing a polymerizable group. As a result, although improvement in lithography performance to some extent has been verified, iodine atoms do not have high organic solvent solubility, and there is a concern about precipitation in a solvent.

Patent Documents 4 and 5 describe a photoacid generator containing a cation having a pentafluorosulfanyl group (—SF₅ group) or a trifluoromethoxy group (—OCF₃ group) introduced therein. As a result, although lithography performance has been improved to some extent, there is still room for improvement, and development of a resist material effective in finer pattern formation has been desired.

### CITATION LIST

Patent Document 1: JP 4425776
Patent Document 2: JP 6720926
Patent Document 3: JP 6973274
Patent Document 4: WO 2023/223624 A
Patent Document 5: JP-A 2022-59112

### SUMMARY OF THE INVENTION

In a chemically amplified resist composition using an acid as a catalyst, it has been desired to develop a resist composition capable of improving the LWR of a line and the CDU of a hole with higher sensitivity.

The invention has been made in view of the above circumstances, and an object thereof is to provide a sulfonium salt monomer that is used for a chemically amplified resist composition having excellent solvent solubility, high sensitivity, and high contrast, and excellent lithography performance such as exposure latitude (EL), LWR, CDU, and depth of focus (DOF), particularly in photolithography using high-energy radiation such as KrF excimer laser light, ArF excimer laser light, an electron beam (EB), and EUV, a polymer containing a repeat unit derived from the sulfonium salt monomer, a chemically amplified resist composition containing the polymer, and a pattern forming process using the chemically amplified resist composition.

As a result of intensive studies to achieve the above object, the inventors have found that a chemically amplified resist composition having high sensitivity, improved lithography performance such as EL, LWR, CDU, and DOF, high contrast, and high resolution can be obtained by using a polymer containing a repeat unit derived from a sulfonium salt monomer containing a sulfonium cation having a —SF₅ group and a sulfonic acid anion containing a polymerizable group as a polymer-bound acid generator, and have completed the invention.

That is, the invention provides a sulfonium salt monomer, a polymer, a chemically amplified resist composition, and a pattern forming process described below.

1. A sulfonium salt monomer having the formula (A):

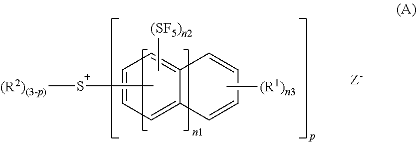

wherein p is 1, 2, or 3, n1 is 0 or 1, n2 is 1 or 2, n3 is 0, 1, 2, or 3, provided that when n1 is 0, 1≤n2+n3≤5, and when n1 is 1, 1≤n2+n3≤7,
R¹ is a halogen atom, a nitro group, a cyano group, a hydroxy group, a C₁-C₂₀ hydrocarbyl group which may contain a heteroatom, a C₁-C₂₀ hydrocarbyloxy group which may contain a heteroatom, or a C₁-C₂₀ hydrocarbylthio group which may contain a heteroatom, when n3 is 2 or 3, a plurality of R¹'s may be identical or different, and two R¹'s may bond together to form a ring with a carbon atom to which they are attached,
R² is a halogen atom or a C₁-C₃₀ hydrocarbyl group which may contain a heteroatom, when p is 1, two R²'s may be identical or different, and two of three substituents attached to S⁺ may bond together to form a ring with a sulfur atom to which they are attached, and
Z⁻ is a fluoroalkanesulfonic acid anion having an aromatic vinyl structure and an iodine atom.
   2. The sulfonium salt monomer of 1, having the formula (A1):

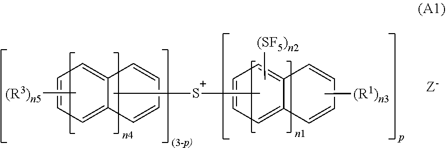

wherein p, n1 to n3, R¹, and Z⁻ are as defined above,
n4 is 0 or 1, n5 is 0,1,2,3,4, or 5,
R³ is a halogen atom, a nitro group, a cyano group, a hydroxy group, a C₁-C₂₀ hydrocarbyl group which may contain a heteroatom, a C₁-C₂₀ hydrocarbyloxy group which may contain a heteroatom, or a C₁-C₂₀ hydrocarbylthio group which may contain a heteroatom, when n5 is 2 to 5, a plurality of R³'s may be identical or different, and two R³'s may bond together to form a ring with a carbon atom to which they are attached.
   3. The sulfonium salt monomer of 1 or 2, wherein Z⁻ is an anion having the formula (Z):

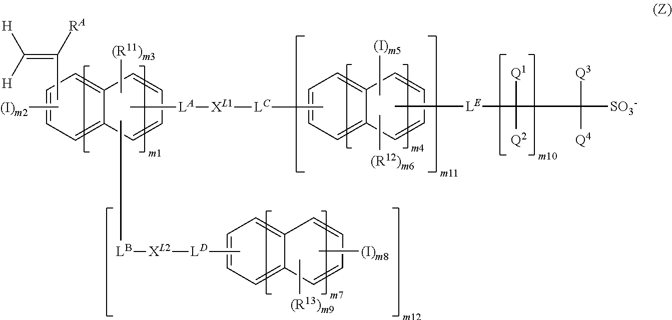

wherein m1 is 0 or 1, m2 is 0, 1, 2, 3, or 4, m3 is 0, 1, 2, or 3, m4 is 0 or 1, m5 is 0, 1, 2, 3, or 4, m6 is 0, 1,2, or 3, m7 is 0 or 1, m8 is 1,2,3, or 4, m9 is 0, 1, 2, or 3, m10 is 0, 1, 2, 3, or 4, m11 is 0 or 1, m12 is 0 or 1, provided that when m1 is 0, 0≤m2+m3+m12≤4, when m1 is 1, 0≤m2+m3+m12≤6, when m4 is 0, 0≤m5+m6≤4, when m4 is 1, 0≤m5+m6≤6, when m7 is 0, 0≤m8+m9≤5, when m7 is 1, 0≤m8+m9≤7, and 1≤m2+m5+m8≤4,
R^{A}'s are each independently a hydrogen atom, a fluorine atom, a methyl group, or a trifluoromethyl group,
R¹¹, R¹², and R¹³ are each independently a halogen atom other than an iodine atom, a nitro group, a hydroxy group, a C₁-C₂₀ hydrocarbyl group which may contain a heteroatom, a C₁-C₂₀ hydrocarbyloxy group which may contain a heteroatom, or a C₁-C₂₀ hydrocarbylthio group which may contain a heteroatom, when m3 is 2 or 3, a plurality of R¹¹'s may be identical or different, and two R¹¹'s may bond together to form a ring with a carbon atom to which they are attached, when m6 is 2 or 3, a plurality of R¹²'s may be identical or different, and two R¹²'s may bond together to form a ring with a carbon atom to which they are attached, when m9 is 2 or 3, a plurality of R¹³'s may be identical or different, and two R¹³'s may bond together to form a ring with a carbon atom to which they are attached,
L^{A}, L^{B}, L^{C}, L^{D}, and L^{E} are each independently a single bond, an ether bond, an ester bond, a sulfonate ester bond, an amide bond, a sulfonamide bond, a carbonate bond, or a carbamate bond,
X^{L1} and X^{L2} are each independently a single bond or a C₁-C₄₀ hydrocarbylene group which may contain a heteroatom,
Q¹ and Q² are each independently a hydrogen atom, a fluorine atom, or a C₁-C₆ fluorinated saturated hydrocarbyl group,
Q³ and Q⁴ are each independently a fluorine atom or a C₁-C₆ fluorinated saturated hydrocarbyl group,
excluding that m11 and m12 are 0 at the same time, and that L^{A}, L^{B}, L^{C}, L^{D}, and
X^{L1} and X^{L2} are a single bond at the same time.
   4. A monomeric photoacid generator containing the sulfonium salt monomer of any one of 1 to 3.
   5. A polymer containing a repeat unit derived from the monomeric photoacid generator of 4.
   6. The polymer of 5, further containing a repeat unit having the formula (a1) or (a2):

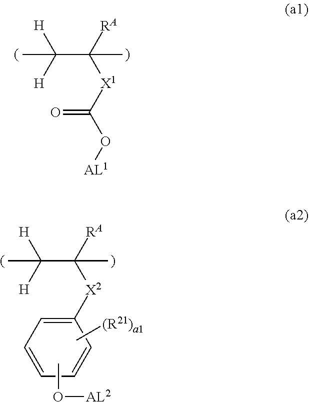

wherein R^{A}'s are each independently a hydrogen atom, a fluorine atom, a methyl group, or a trifluoromethyl group,
X¹ is a single bond, a phenylene group, a naphthylene group, or *—C(═O)—O—X¹¹—, and the phenylene group or the naphthylene group may be substituted with a hydroxy group, a nitro group, a cyano group, a C₁-C₁₀ saturated hydrocarbyl group which may contain a fluorine atom, a C₁-C₁₀ saturated hydrocarbyloxy group which may contain a fluorine atom, or a halogen atom, X¹¹ is a C₁-C₁₀ saturated hydrocarbylene group, a phenylene group, or a naphthylene group, and the saturated hydrocarbylene group may contain a hydroxy group, an ether bond, an ester bond, or a lactone ring,
X² is a single bond or *—C(═O)—O—,
designates a point of attachment to a carbon atom in the backbone,
R²¹ is a halogen atom, a cyano group, a hydroxy group, a nitro group, a C₁-C₂₀ hydrocarbyl group which may contain a heteroatom, a C₁-C₂₀ hydrocarbyloxy group which may contain a heteroatom, a C₂-C₂₀ hydrocarbylcarbonyl group which may contain a heteroatom, a C₂-C₂₀ hydrocarbylcarbonyloxy group which may contain a heteroatom, or a C₂-C₂₀ hydrocarbyloxycarbonyl group which may contain a heteroatom, when a1 is 2 or more, a plurality of R²¹'s may be identical or different,
AL¹ and AL² are each independently an acid labile group, and
a1 is 0, 1, 2, 3, or 4.
   7. The polymer of 5 or 6, further containing a repeat unit having the formula (a3):

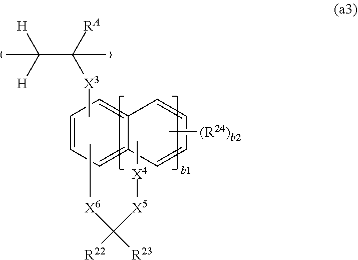

wherein b1 is 0 or 1, b2 is 0, 1, 2, or 3 when b1 is 0, and is 0, 1, 2, 3, 4, or 5 when b1 is 1,
R^{A} is a hydrogen atom, a fluorine atom, a methyl group, or a trifluoromethyl group,
X³ is a single bond, *—C(═O)—O—, or *—C(═O)—NH—, * designates a point of attachment to a carbon atom in the backbone,
R²² and R²³ are each independently a hydrogen atom or a C₁-C₂₀ hydrocarbyl group which may contain a heteroatom, and R²² and R²³ may bond together to form a ring with a carbon atom to which they are attached,
R²⁴ is a halogen atom, a hydroxy group, a cyano group, a nitro group, a C₁-C₂₀ hydrocarbyl group which may contain a heteroatom, a C₁-C₂₀ hydrocarbyloxy group which may contain a heteroatom, a C₂-C₂₀ hydrocarbyloxycarbonyl group which may contain a heteroatom, a C₁-C₂₀ hydrocarbylthio group which may contain a heteroatom, or —N(R^{24A})(R^{24B}), R^{24A} and R^{24B} are each independently a hydrogen atom or a C₁-C₆ hydrocarbyl group, when b2 is 2 or more, a plurality of R²⁴'s may be identical or different, and a plurality of R²⁴'s may bond together to form a ring with a carbon atom on an aromatic ring to which they are attached,
X⁴ is a single bond, a C₁-C₄ aliphatic hydrocarbylene group, a carbonyl group, a sulfonyl group, or a group obtained by combining these groups, and
X⁵ and X⁶ are each independently an oxygen atom or a sulfur atom, provided that X⁴ and X⁶ are attached to an adjacent carbon atom on an aromatic ring.
   8. The polymer of any one of 5 to 7, further containing a repeat unit having the formula (b1) or (b2):

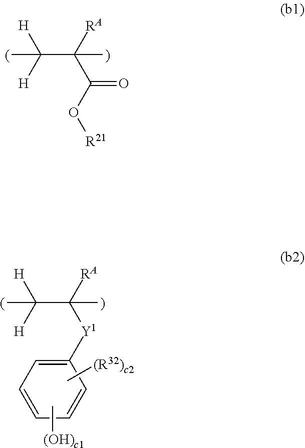

wherein R^{A}'s are each independently a hydrogen atom, a fluorine atom, a methyl group, or a trifluoromethyl group,
Y¹ is a single bond or *—C(═O)—O—, * designates a point of attachment to a carbon atom in the backbone,
R³¹ is a hydrogen atom or a C₁-C₂₀ group containing at least one or more structures selected from a hydroxy group other than a phenolic hydroxy group, a cyano group, a carbonyl group, a carboxy group, an ether bond, an ester bond, a sulfonate ester bond, a carbonate bond, a lactone ring, a sultone ring, and a carboxylic anhydride (—C(═O)—O—C(═O)—),
R³² is a halogen atom, a hydroxy group, a nitro group, a C₁-C₂₀ hydrocarbyl group which may contain a heteroatom, a C₁-C₂₀ hydrocarbyloxy group which may contain a heteroatom, a C₂-C₂₀ hydrocarbylcarbonyl group which may contain a heteroatom, a C₂-C₂₀ hydrocarbylcarbonyloxy group which may contain a heteroatom, or a C₂-C₂₀ hydrocarbyloxycarbonyl group which may contain a heteroatom, when c2 is 2 or more, a plurality of R³²'s may be identical or different,
c1 is an integer of 1 to 4, and c2 is an integer of 0 to 4, provided that 1≤b+c≤5.
   9. A chemically amplified resist composition containing (A) a base polymer containing the polymer of any one of 5 to 8.
   10. The chemically amplified resist composition of 9, further containing (B) an organic solvent.
   11. The chemically amplified resist composition of 9 or 10, further containing (C) a quencher.
   12. The chemically amplified resist composition of any one of 9 to 11, further containing (D) a photoacid generator.
   13. The chemically amplified resist composition of any one of 9 to 12, further containing (E) a surfactant.
   14. A pattern forming process including the steps of forming a resist film on a substrate using the chemically amplified resist composition of any one of 9 to 13, exposing the resist film to high-energy radiation, and developing the exposed resist film using a developer.
   15. The pattern forming process of 14, wherein the high-energy radiation is KrF excimer laser light, ArF excimer laser light, EB, or EUV with a wavelength of 3 to 15 nm.

### Advantageous Effects of the Invention

When a pattern is formed using a polymer containing the sulfonium salt monomer of the invention as a photoacid generator and a chemically amplified resist composition, a resist pattern having high contrast and good sensitivity and excellent lithography performance such as EL, LWR, CDU, and DOF can be formed.

### DESCRIPTION OF THE PREFERRED EMBODIMENT

Hereinafter, the invention is described in detail. In the following description, depending on a structure having a chemical formula, an asymmetric carbon may exist, and an enantiomer or a diastereomer may exist, and in that case, those isomers are represented by one formula as a representative. One type of these isomers may be used alone, or two or more types of isomers may be used as a mixture.

### Sulfonium Salt Monomer

The sulfonium salt monomer of the invention has the formula (A):

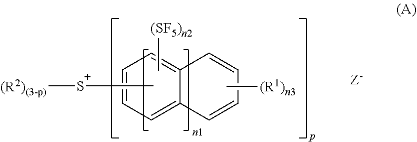

In the formula (A), p is 1, 2, or 3.

In the formula (A), n1 is 0 or 1. The relevant structure is a benzene ring when n1 is 0 and a naphthalene ring when n1 is 1, and from the aspect of solvent solubility, a benzene ring when n1 is 0 is preferred. n2 is 1 or 2. From the aspect of raw material procurement, n2 is preferably 1. n3 is 0, 1, 2, or 3. From the aspect of raw material procurement, n3 is preferably 0, 1, or 2. However, when n1 is 0, 1≤n2+n3≤5, and when n1 is 1, 1≤n2+n3≤7.

In the formula (A), R¹ is a halogen atom, a nitro group, a cyano group, a hydroxy group, a C₁-C₂₀ hydrocarbyl group which may contain a heteroatom, a C₁-C₂₀ hydrocarbyloxy group which may contain a heteroatom, or a C₁-C₂₀ hydrocarbylthio group which may contain a heteroatom. Examples of the halogen atom include a fluorine atom, a chlorine atom, a bromine atom, and an iodine atom. The hydrocarbyl group and the hydrocarbyl moiety of the hydrocarbyloxy group and the hydrocarbylthio group may be saturated or unsaturated and straight, branched, or cyclic. Specific examples thereof include C₁-C₂₀ alkyl groups such as a methyl group, an ethyl group, a n-propyl group, an isopropyl group, a n-butyl group, an isobutyl group, a sec-butyl group, a tert-butyl group, a n-pentyl group, a n-hexyl group, a n-octyl group, a n-nonyl group, a n-decyl group, an undecyl group, a dodecyl group, a tridecyl group, a tetradecyl group, a pentadecyl group, a heptadecyl group, an octadecyl group, a nonadecyl group, and an icosyl group; C₃-C₂₀ cyclic saturated hydrocarbyl groups such as a cyclopropyl group, a cyclopentyl group, a cyclohexyl group, a cyclopropylmethyl group, a 4-methylcyclohexyl group, a cyclohexylmethyl group, a norbornyl group, and an adamantyl group; C₂-C₂₀ alkenyl groups such as a vinyl group, a 1-propenyl group, a 2-propenyl group, a butenyl group, and a hexenyl group; C₃-C₂₀ cyclic unsaturated hydrocarbyl groups such as a cyclohexenyl group; C₆-C₂₀ aryl groups such as a phenyl group and a naphthyl group; C₇-C₂₀ aralkyl groups such as a benzyl group, a 1-phenylethyl group, and a 2-phenylethyl group; and a group obtained by combining these groups. Among them, an aryl group is preferred. Some or all hydrogen atoms in the hydrocarbyl group may be substituted with a group containing a heteroatom such as an oxygen atom, a sulfur atom, a nitrogen atom, or a halogen atom, and some —CH₂— in the hydrocarbyl group may be substituted with a group containing a heteroatom such as an oxygen atom, a sulfur atom, or a nitrogen atom, so that the hydrocarbyl group may contain a hydroxy group, a cyano group, a fluorine atom, a chlorine atom, a bromine atom, an iodine atom, a carbonyl group, an ether bond, an ester bond, a sulfonate ester bond, a carbonate bond, a lactone ring, a sultone ring, a carboxylic anhydride (—C(═O)—O—C(═O)—), a haloalkyl group, or the like. When n3 is 2 or 3, a plurality of R's may be identical or different. Further, when n3 is 2 or 3, a plurality of R¹'s may bond together to form a ring with a carbon atom to which they are attached. The ring is preferably a 5- to 8-membered ring.

In the formula (A), R² is a halogen atom or a C₁-C₃₀ hydrocarbyl group which may contain a heteroatom. When p is 1, two R²'s may be identical or different.

Specific examples of the halogen atom of R² include a fluorine atom, a chlorine atom, a bromine atom, and an iodine atom.

The hydrocarbyl group of R² may be saturated or unsaturated and straight, branched, or cyclic. Specific examples thereof include C₁-C₃₀ alkyl groups such as a methyl group, an ethyl group, a n-propyl group, an isopropyl group, a n-butyl group, an isobutyl group, a sec-butyl group, and a tert-butyl group; C₃-C₃₀ cyclic saturated hydrocarbyl groups such as a cyclopropyl group, a cyclopentyl group, a cyclohexyl group, a cyclopropylmethyl group, a 4-methylcyclohexyl group, a cyclohexylmethyl group, a norbornyl group, and an adamantyl group; C₂-C₃₀ alkenyl groups such as a vinyl group, a 1-propenyl group, a 2-propenyl group, a butenyl group, and a hexenyl group; C₃-C₃₀ cyclic unsaturated hydrocarbyl groups such as a cyclohexenyl group; C₆-C₃₀ aryl groups such as a phenyl group, a naphthyl group, and a thienyl group; C₇-C₃₀ aralkyl groups such as a benzyl group, a 1-phenylethyl group, and a 2-phenylethyl group; and a group obtained by combining these groups, and aryl groups are preferred. Some or all hydrogen atoms in the hydrocarbyl group may be substituted with a group containing a heteroatom such as an oxygen atom, a sulfur atom, a nitrogen atom, or a halogen atom, and some —CH₂— in the hydrocarbyl group may be substituted with a group containing a heteroatom such as an oxygen atom, a sulfur atom, or a nitrogen atom, so that the hydrocarbyl group may contain a hydroxy group, a fluorine atom, a chlorine atom, a bromine atom, an iodine atom, a cyano group, a nitro group, a carbonyl group, an ether bond, an ester bond, a sulfonate ester bond, a carbonate bond, a lactone ring, a sultone ring, a carboxylic anhydride (—C(═O)—O—C(═O)—), a haloalkyl group, or the like.

Two of three substituents attached to S⁺ may bond together to form a ring with a sulfur atom to which they are attached. At this time, specific examples of the structure of the ring include those having the following formulae.

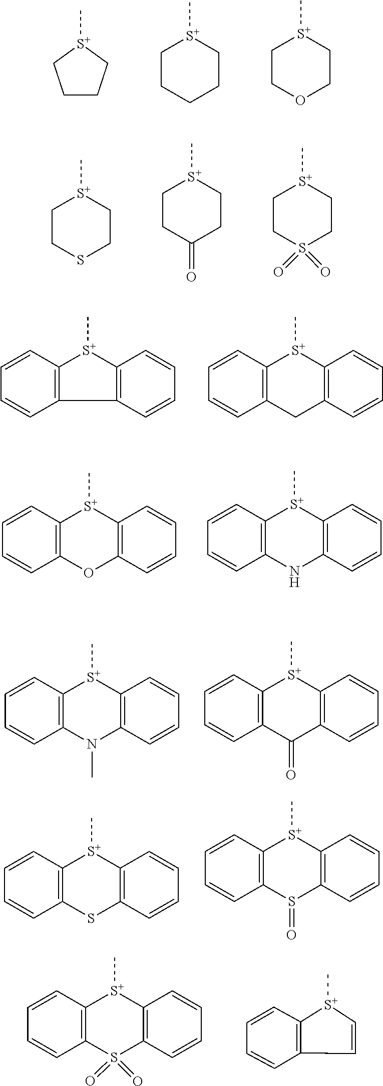

In the formulae, a broken line designates a point of attachment.

As the sulfonium salt monomer having the formula (A), a sulfonium salt monomer having the formula (A1) is preferred.

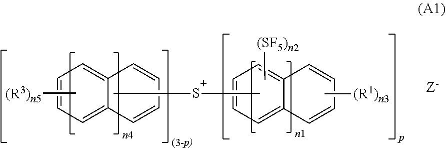

In the formula, p, n1 to n3, R¹, and Z⁻ are as defined above.

In the formula (A1), n4 is 0 or 1. The relevant structure is a benzene ring when n4 is 0 and a naphthalene ring when n4 is 1, and from the aspect of solvent solubility, a benzene ring when n4 is 0 is preferred. n5 is 0, 1, 2, 3, 4, or 5. From the aspect of raw material procurement, n5 is preferably 0, 1, or 2.

In the formula (A1), R³ is a halogen atom, a nitro group, a cyano group, a hydroxy group, a C₁-C₂₀ hydrocarbyl group which may contain a heteroatom, a C₁-C₂₀ hydrocarbyloxy group which may contain a heteroatom, or a C₁-C₂₀ hydrocarbylthio group which may contain a heteroatom. The hydrocarbyl group and the hydrocarbyl moiety of the hydrocarbyloxy group and the hydrocarbylthio group may be saturated or unsaturated and straight, branched, or cyclic. Specific examples thereof are as exemplified above for the hydrocarbyl group of R¹, but not limited thereto. When n5 is 2 to 5, a plurality of R³'s may be identical or different, and two R³'s may bond together to form a ring with a carbon atom to which they are attached.

Examples of the cation of the sulfonium salt monomer having the formula (1) include those shown below, but are not limited thereto.

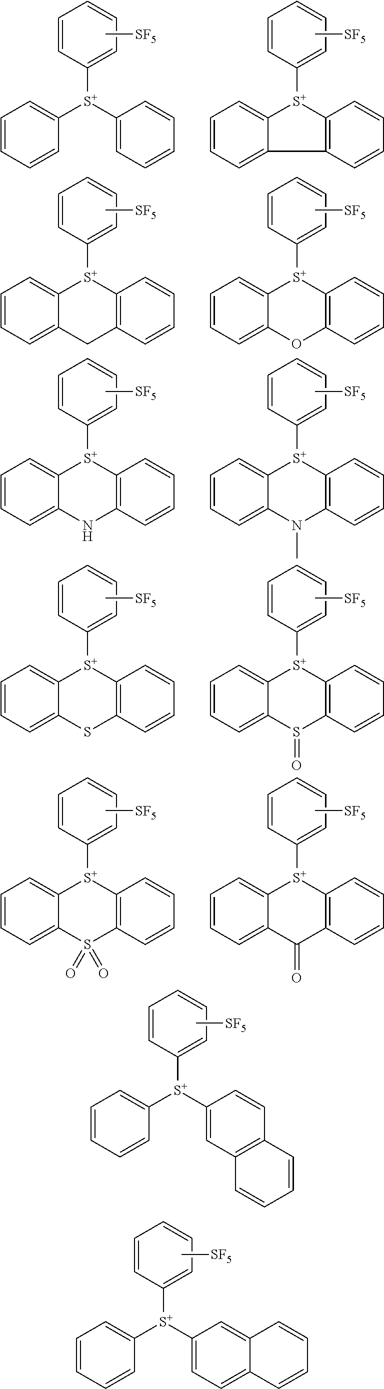

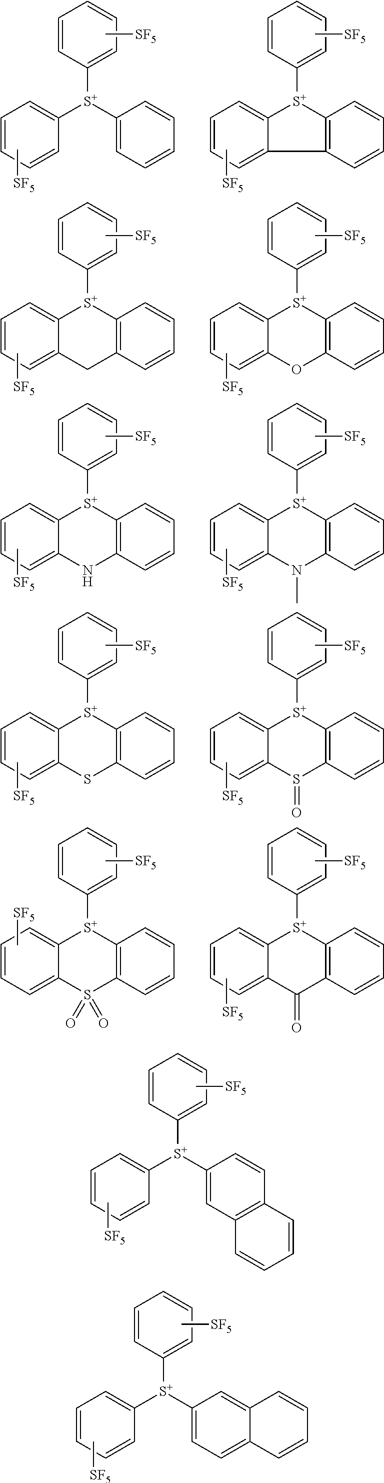

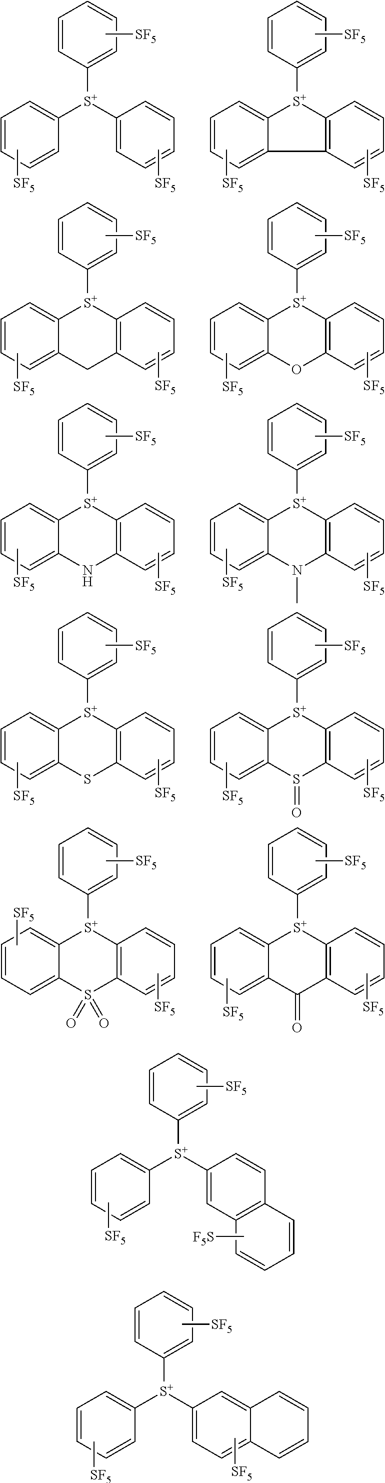

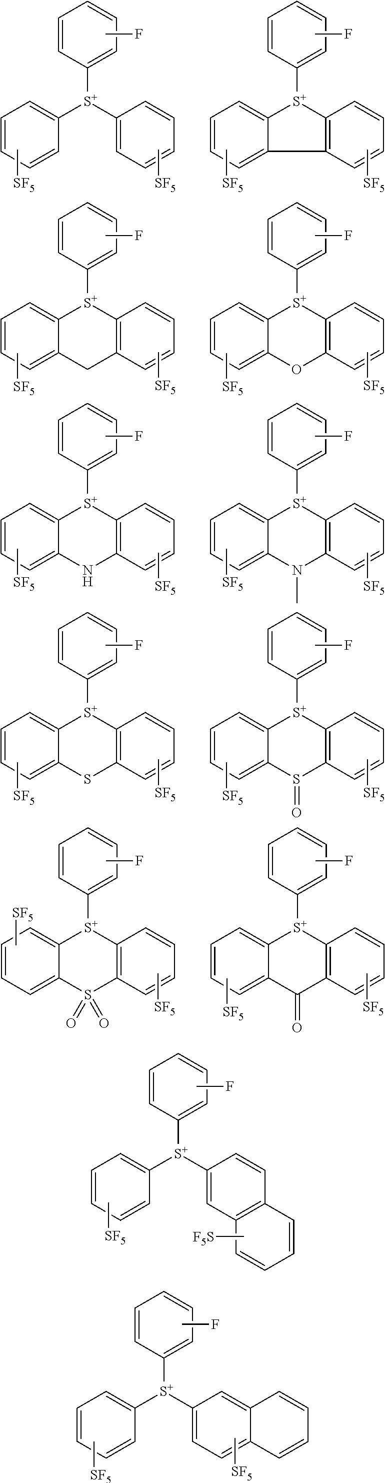

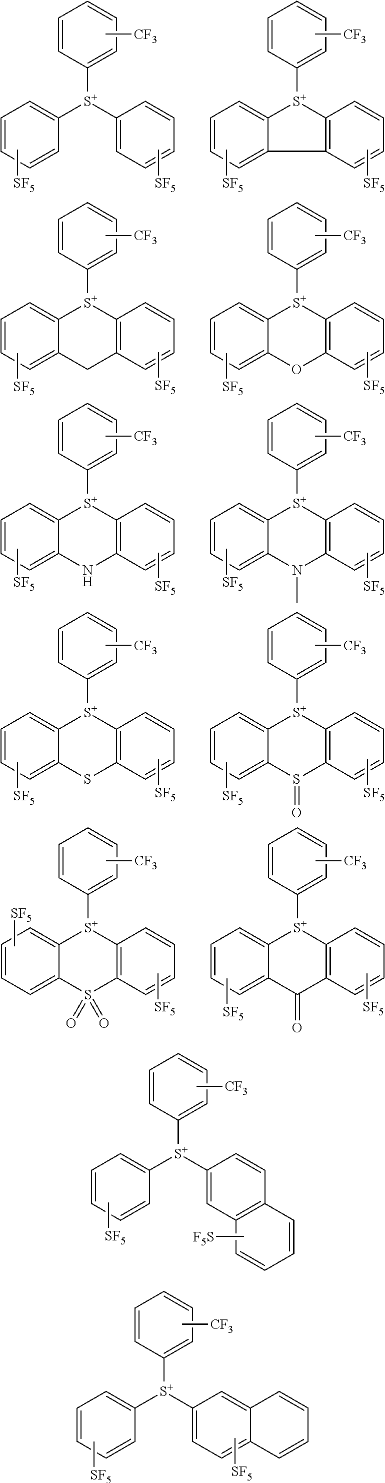

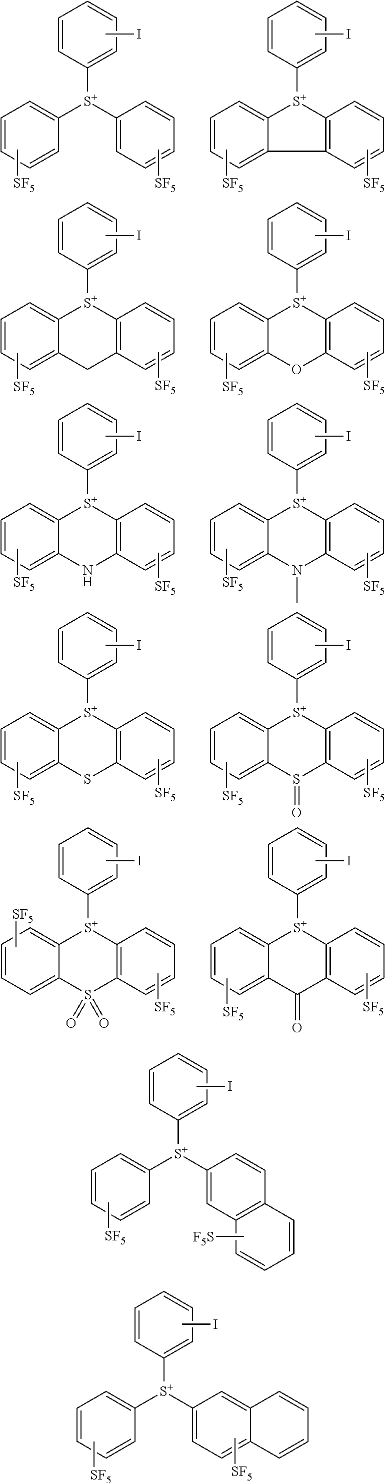

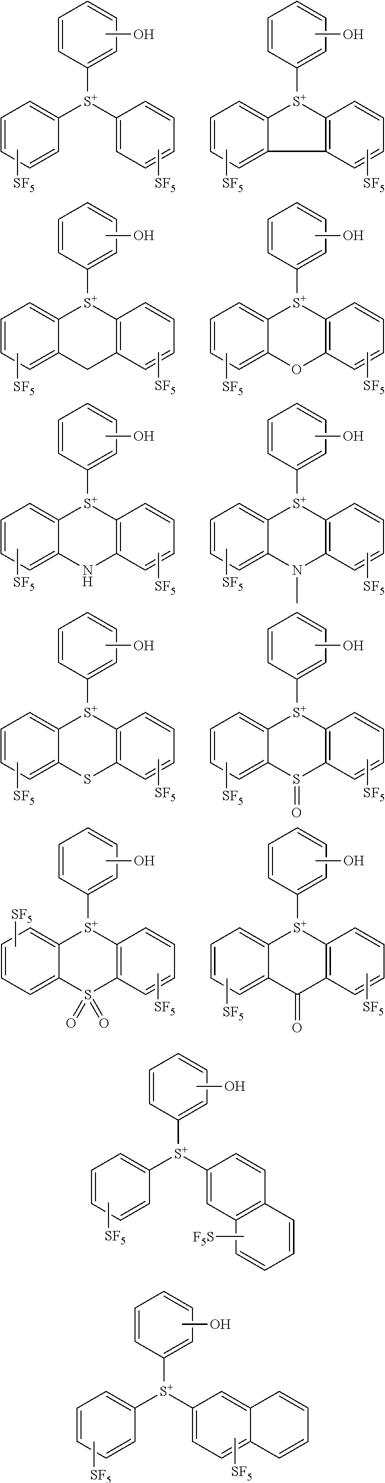

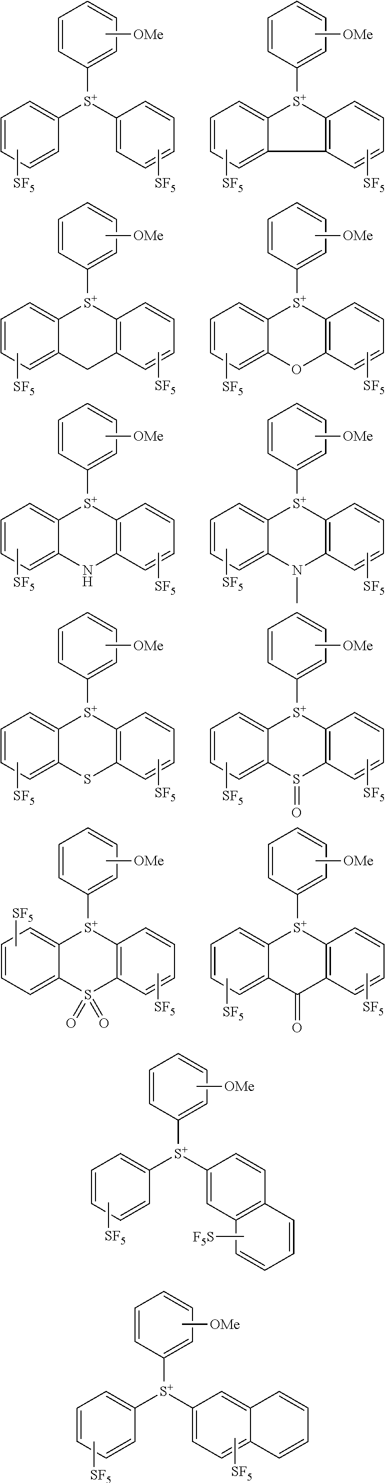

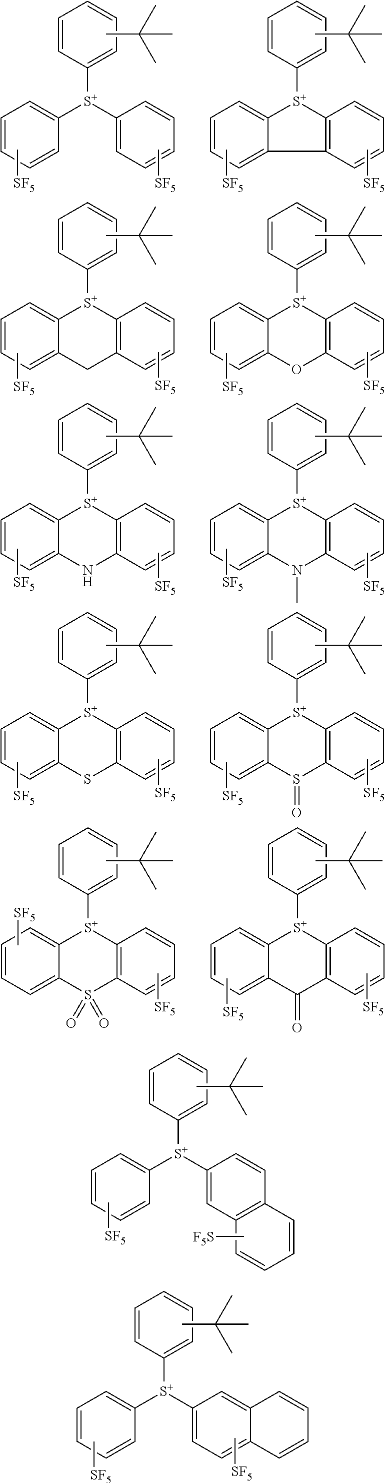

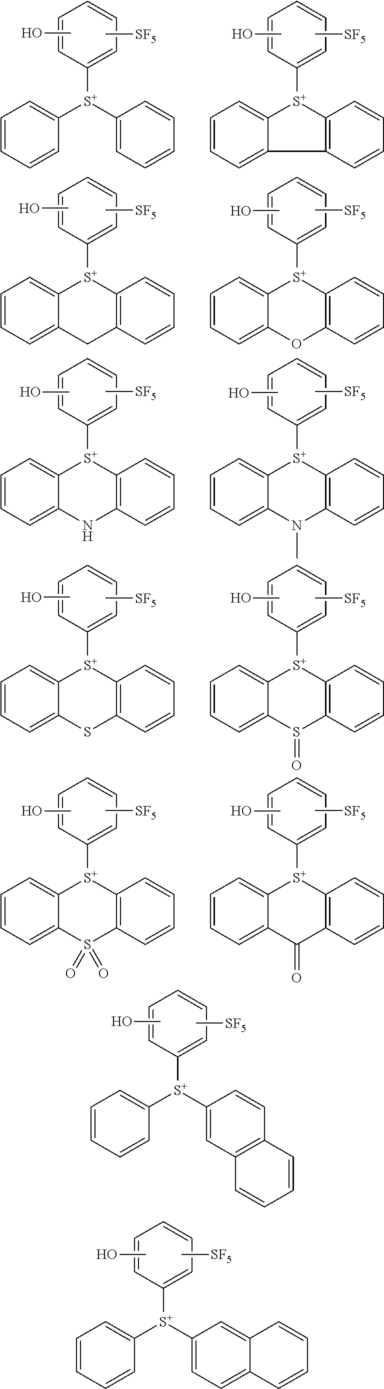

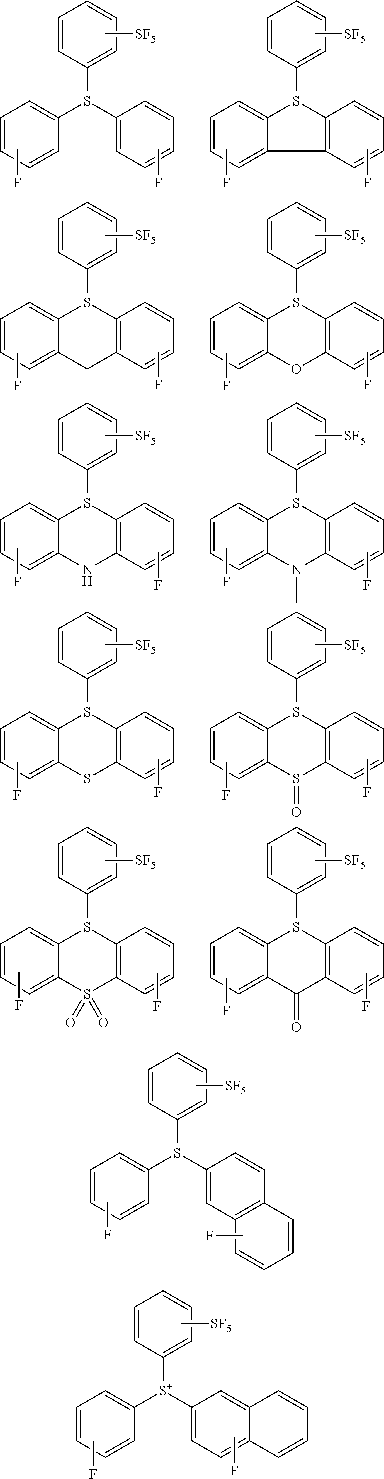

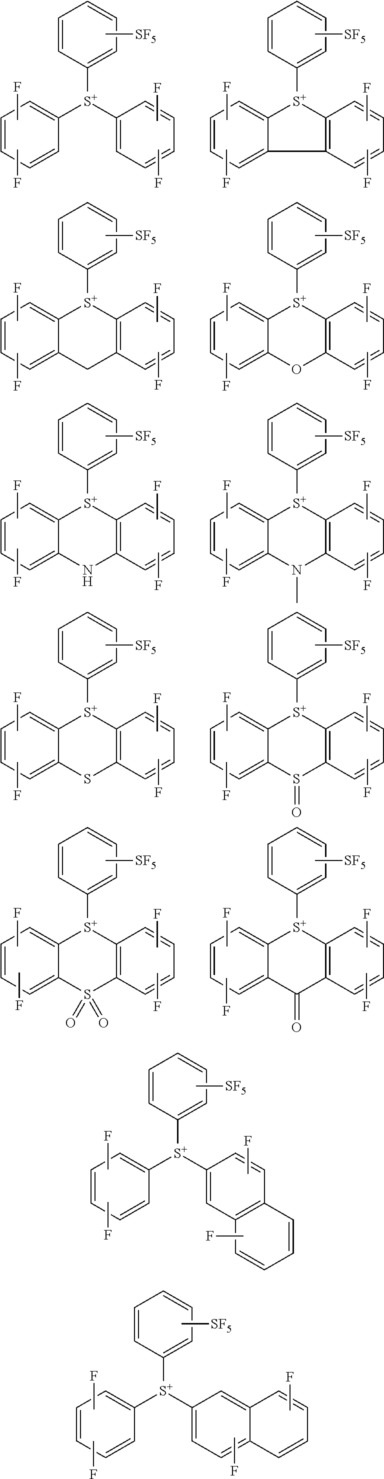

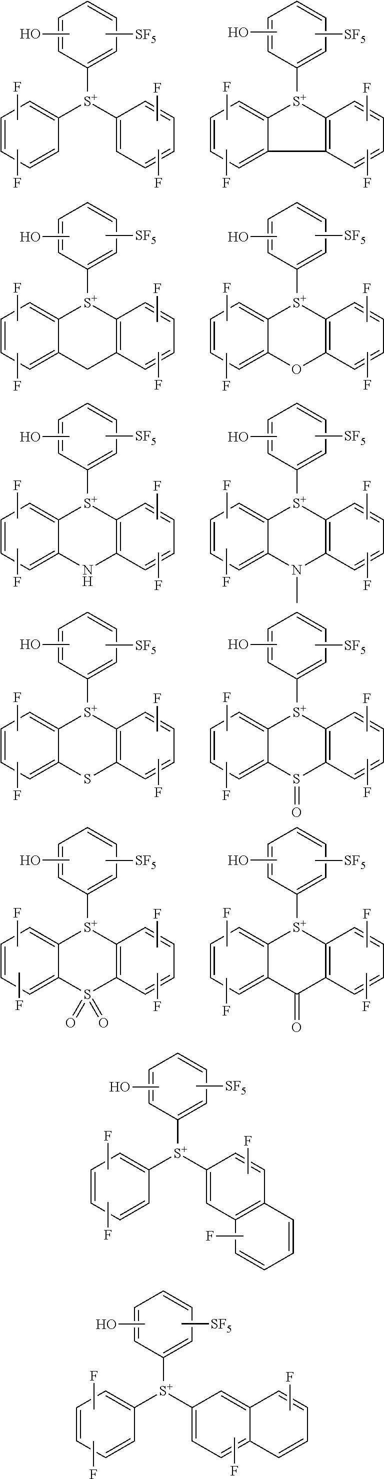

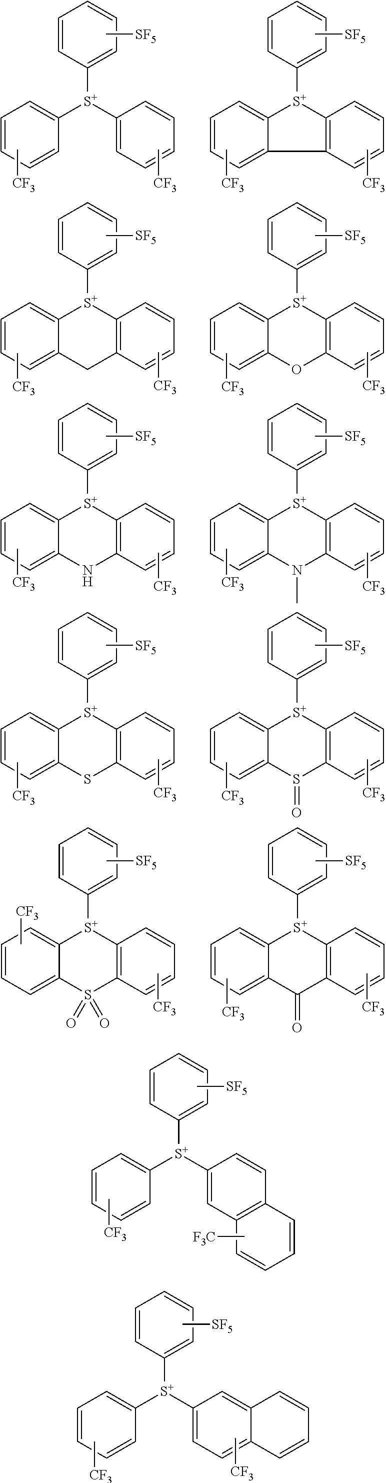

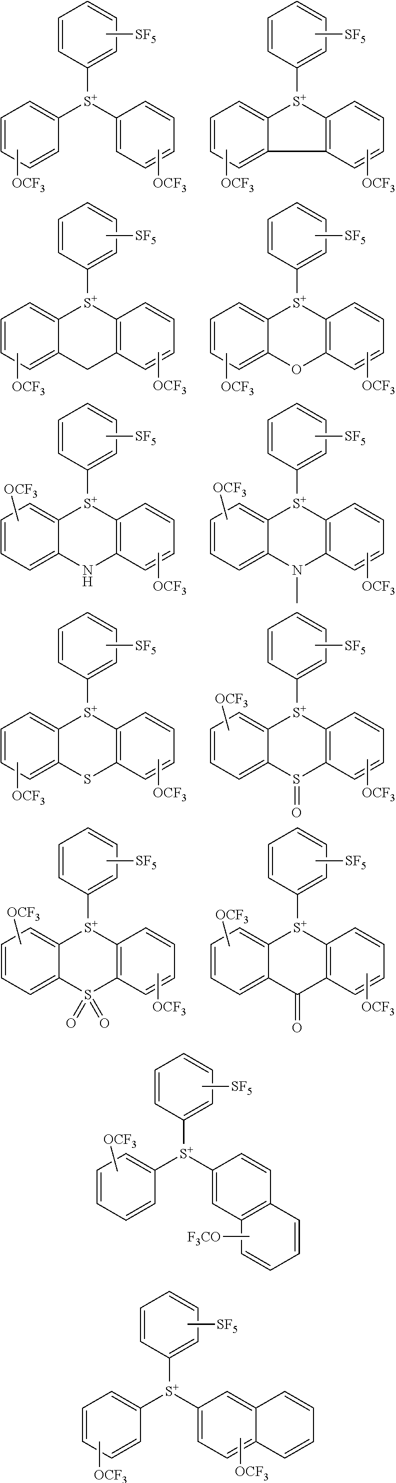

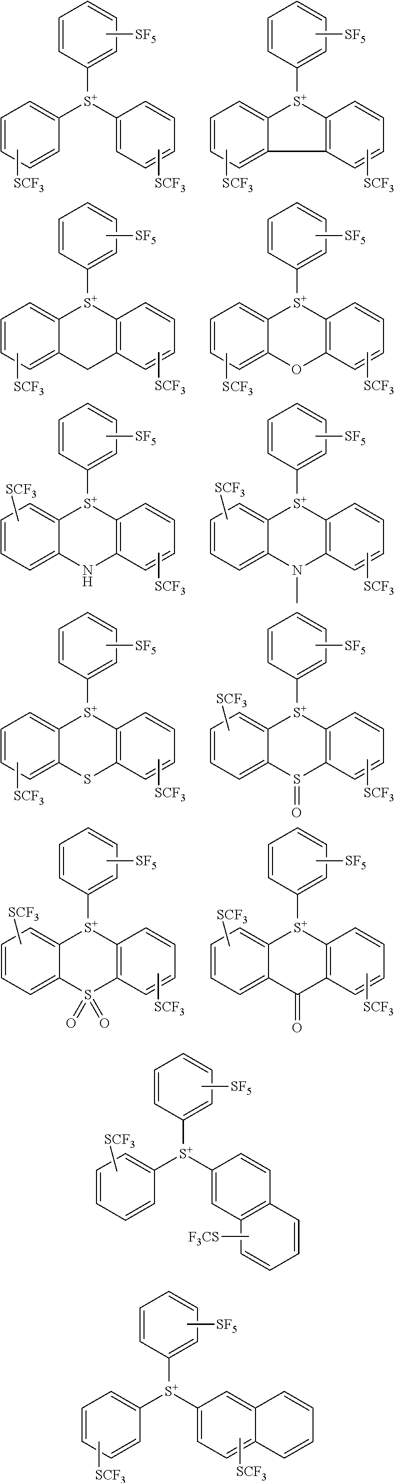

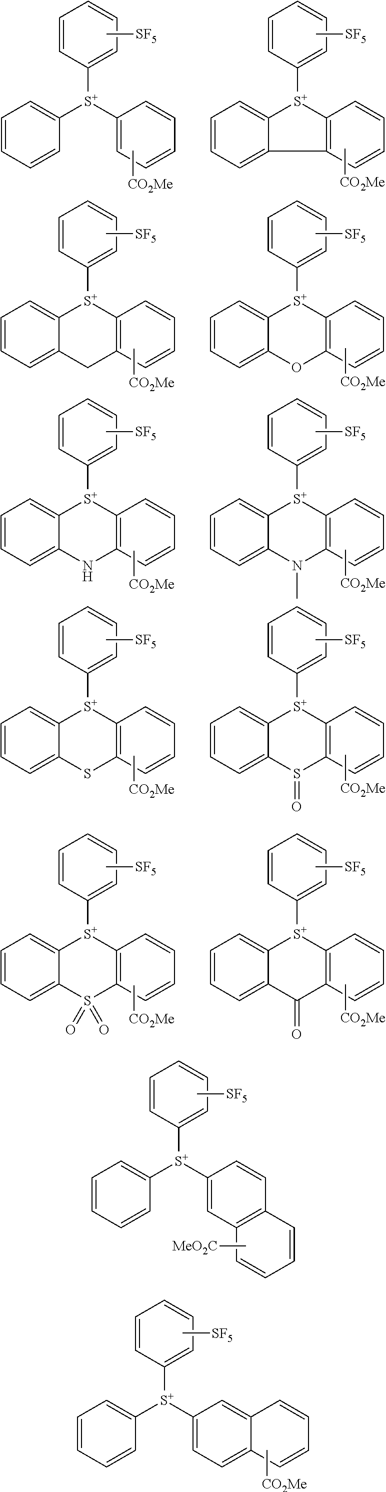

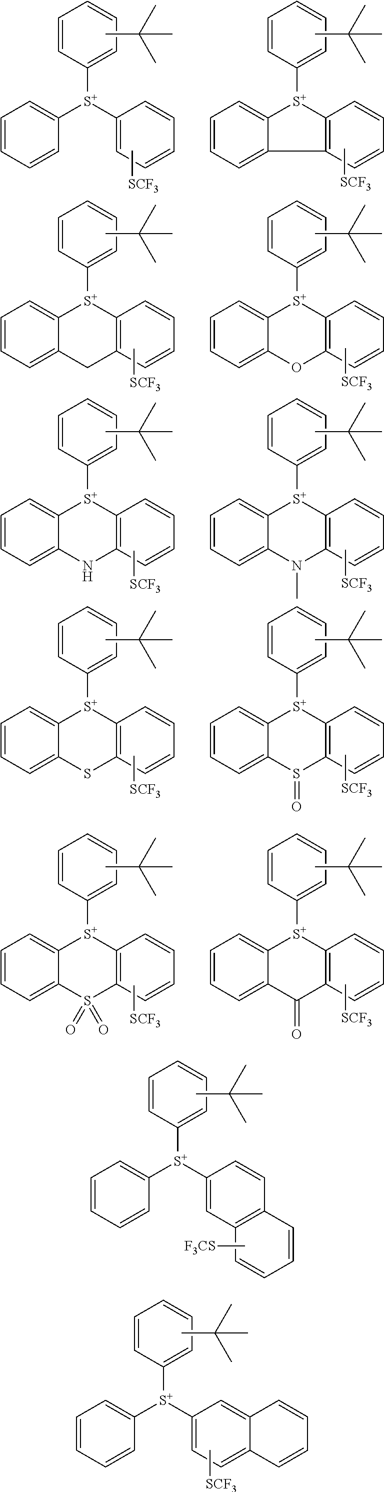

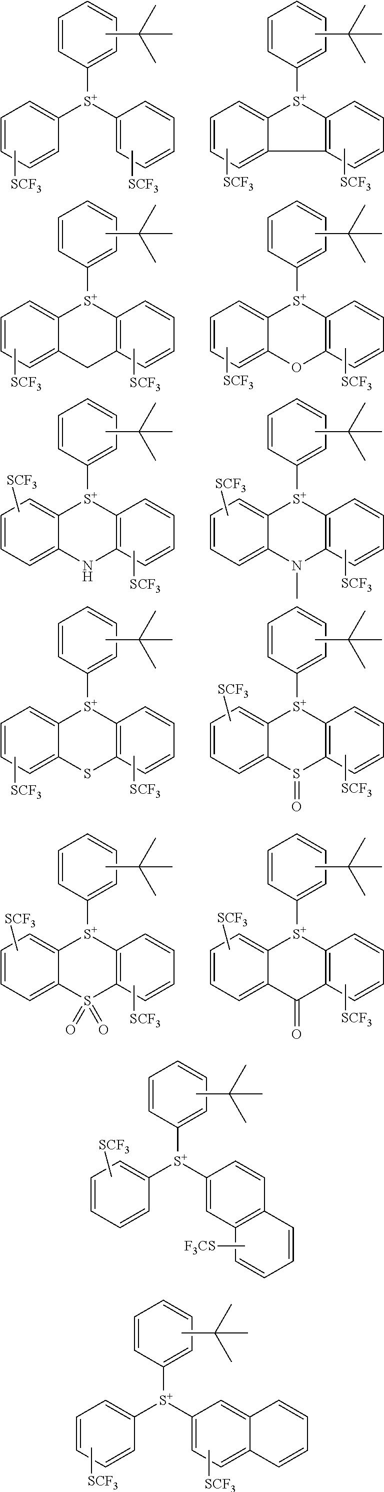

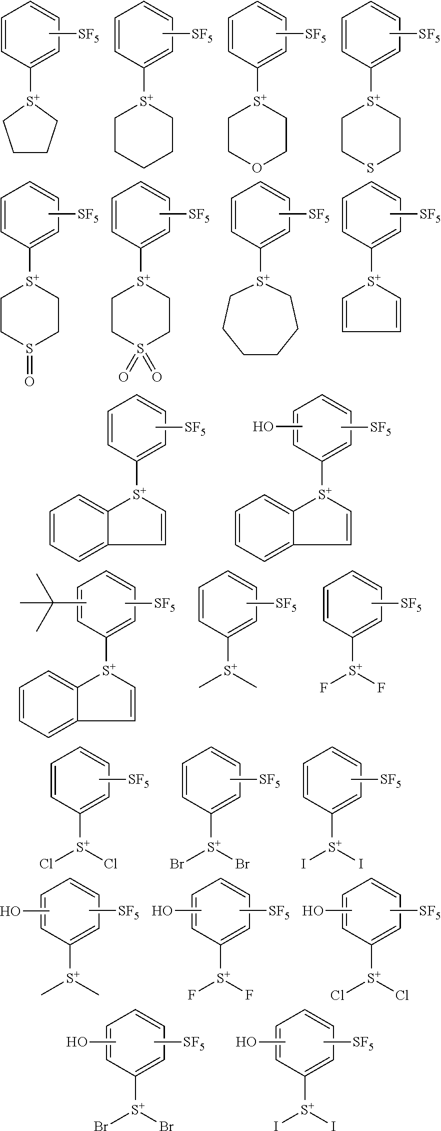

In the formula (A), Y⁻ is a fluoroalkanesulfonic acid anion having an aromatic vinyl structure and an iodine atom. As the fluoroalkanesulfonic acid anion, a fluoroalkanesulfonic acid anion having the formula (Z) is preferred.

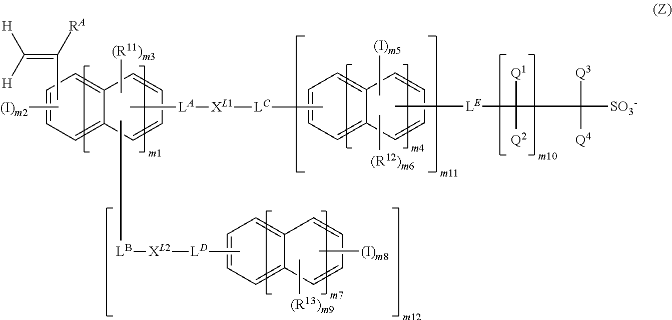

In the formula (Z), m1 is 0 or 1. The relevant structure is a benzene ring when m1 is 0 and a naphthalene ring when m1 is 1, and from the aspect of solvent solubility, a benzene ring when m1 is 0 is preferred. m2 is 0, 1, 2, 3, or 4. From the aspect of raw material procurement, m2 is preferably 0, 1, 2, or 3, more preferably 0, 1, or 2, and still more preferably 0 or 1. m3 is 0, 1, 2, or 3.

In the formula (Z), m4 is 0 or 1. The relevant structure is a benzene ring when m4 is 0 and a naphthalene ring when m4 is 1, and from the aspect of solvent solubility, a benzene ring when m4 is 0 is preferred. m5 is 0, 1, 2, 3, or 4. From the aspect of raw material procurement, m5 is preferably 0, 1, 2, or 3, and more preferably 0, 1, or 2. m6 is 0, 1, 2, or 3.

In the formula (Z), m7 is 0 or 1. The relevant structure is a benzene ring when m7 is 0 and a naphthalene ring when m7 is 1, and from the aspect of solvent solubility, a benzene ring when m7 is 0 is preferred. m8 is 1, 2, 3, or 4. From the aspect of raw material procurement, m8 is preferably 1, 2, or 3, and more preferably 1 or 2. m9 is 0, 1, 2, or 3.

In the formula (Z), m10 is 0, 1, 2, 3, or 4, but is preferably 0, 1, 2, or 3, more preferably 1, 2, or 3, and still more preferably 1. m11 is 0 or 1. m12 is 0 or 1.

When m1 is 0, 0≤m2+m3+m12≤4, and when m1 is 1, 0≤m2+m3+m12≤6. When m4 is 0, 0≤m5+m6≤4, and when m4 is 1, 0≤m5+m6≤6. When m7 is 0, 0≤m8+m9≤5, and when m7 is 1, 0≤m8+m9≤7. In addition, with respect to the number of iodine atoms in the anion, the larger the number of iodine atoms is, the more the absorption to EUV is particularly enhanced, but since there is a concern that the solvent solubility becomes poor and precipitation occurs in the resist composition, it is preferred that 1≤m2+m5+m8≤4.

In the formula (Z), R^{A} is a hydrogen atom, a fluorine atom, a methyl group, or a trifluoromethyl group. Among them, a hydrogen atom and a methyl group are preferred, and a hydrogen atom is more preferred.

In the formula (Z), R¹¹ is a halogen atom other than an iodine atom, a nitro group, a hydroxy group, a C₁-C₂₀ hydrocarbyl group which may contain a heteroatom, a C₁-C₂₀ hydrocarbyloxy group which may contain a heteroatom, or a C₁-C₂₀ hydrocarbylthio group which may contain a heteroatom. The halogen atom other than the iodine atom is preferably a fluorine atom, a chlorine atom, or a bromine atom, and more preferably a fluorine atom. The hydrocarbyl group and the hydrocarbyl moiety of the hydrocarbyloxy group and the hydrocarbylthio group may be saturated or unsaturated and straight, branched, or cyclic. Specific examples thereof include C₁-C₂₀ alkyl groups such as a methyl group, an ethyl group, a n-propyl group, an isopropyl group, a n-butyl group, an isobutyl group, a sec-butyl group, a tert-butyl group, a n-pentyl group, a n-hexyl group, a n-octyl group, a n-nonyl group, a n-decyl group, an undecyl group, a dodecyl group, a tridecyl group, a tetradecyl group, a pentadecyl group, a heptadecyl group, an octadecyl group, a nonadecyl group, and an icosyl group; C₃-C₂₀ cyclic saturated hydrocarbyl groups such as a cyclopropyl group, a cyclopentyl group, a cyclohexyl group, a cyclopropylmethyl group, a 4-methylcyclohexyl group, a cyclohexylmethyl group, a norbornyl group, and an adamantyl group; C₂-C₂₀ alkenyl groups such as a vinyl group, a 1-propenyl group, a 2-propenyl group, a butenyl group, and a hexenyl group; C₃-C₂₀ cyclic unsaturated hydrocarbyl groups such as a cyclohexenyl group; C₆-C₂₀ aryl groups such as a phenyl group and a naphthyl group; C₇-C₂₀ aralkyl groups such as a benzyl group, a 1-phenylethyl group, and a 2-phenylethyl group; and a group obtained by combining these groups. Some or all hydrogen atoms in the hydrocarbyl group may be substituted with a group containing a heteroatom such as an oxygen atom, a sulfur atom, a nitrogen atom, or a halogen atom, and some —CH₂— in the hydrocarbyl group may be substituted with a group containing a heteroatom such as an oxygen atom, a sulfur atom, or a nitrogen atom, so that the hydrocarbyl group may contain a hydroxy group, a cyano group, a fluorine atom, a chlorine atom, a bromine atom, an iodine atom, a carbonyl group, an ether bond, an ester bond, a sulfonate ester bond, a carbonate bond, a lactone ring, a sultone ring, a carboxylic anhydride (—C(═O)—O—C(═O)—), a haloalkyl group, or the like. When m3 is 2, a plurality of R¹¹'s may be identical or different.

Further, when m3 is 2 or 3, two R¹¹'s may bond together to form a ring with a carbon atom to which they are attached. Specific examples of the ring formed at this time include a cyclopropane ring, a cyclobutane ring, a cyclopentane ring, a cyclohexane ring, a norbornane ring, and an adamantane ring. Some or all hydrogen atoms in the ring may be substituted with a group containing a heteroatom such as an oxygen atom, a sulfur atom, a nitrogen atom, or a halogen atom, and some —CH₂— in the ring may be substituted with a group containing a heteroatom such as an oxygen atom, a sulfur atom, or a nitrogen atom, so that the ring may contain a hydroxy group, a fluorine atom, a chlorine atom, a bromine atom, an iodine atom, a cyano group, a carbonyl group, an ether bond, an ester bond, a sulfonate ester bond, a carbonate bond, a lactone ring, a sultone ring, a carboxylic anhydride (—C(═O)—O—C(═O)—), a haloalkyl group, or the like.

In the formula (Z), R¹² is a halogen atom other than an iodine atom, a nitro group, a hydroxy group, a C₁-C₂₀ hydrocarbyl group which may contain a heteroatom, a C₁-C₂₀ hydrocarbyloxy group which may contain a heteroatom, or a C₁-C₂₀ hydrocarbylthio group which may contain a heteroatom. Specific examples of the halogen atom other than an iodine atom include a fluorine atom, a chlorine atom, and a bromine atom. The hydrocarbyl group and the hydrocarbyl moiety of the hydrocarbyloxy group and the hydrocarbylthio group may be saturated or unsaturated and straight, branched, or cyclic. Specific examples thereof are as exemplified above for the hydrocarbyl group of R¹¹, but not limited thereto. When m6 is 2 or 3, a plurality of R¹²'s may be identical or different.

Further, when m6 is 2, two R¹²'s may bond together to form a ring with a carbon atom to which they are attached. The ring is preferably a 5- to 8-membered ring.

In the formula (Z), R¹³ is a halogen atom other than an iodine atom, a nitro group, a hydroxy group, a C₁-C₂₀ hydrocarbyl group which may contain a heteroatom, a C₁-C₂₀ hydrocarbyloxy group which may contain a heteroatom, or a C₁-C₂₀ hydrocarbylthio group which may contain a heteroatom. Specific examples of the halogen atom other than an iodine atom include a fluorine atom, a chlorine atom, and a bromine atom. The hydrocarbyl group and the hydrocarbyl moiety of the hydrocarbyloxy group and the hydrocarbylthio group may be saturated or unsaturated and straight, branched, or cyclic. Specific examples thereof are as exemplified above for the hydrocarbyl group of R¹¹, but not limited thereto. When m9 is 2 or 3, a plurality of R¹³'s may be identical or different.

Further, when m9 is 2, two R¹³'s may bond together to form a ring with a carbon atom to which they are attached. The ring is preferably a 5- to 8-membered ring.

In the formula (Z), L^{A}, L^{B}, L^{C}, L^{D}, and L^{E} are each independently a single bond, an ether bond, an ester bond, a sulfonate ester bond, an amide bond, a sulfonamide bond, a carbonate bond, or a carbamate bond. Among them, L^{A} is preferably a single bond, an ether bond, an ester bond, or a sulfonate ester bond, and more preferably an ether bond, an ester bond, or a sulfonate ester bond. L^{B} is preferably a single bond, an ether bond, an ester bond, an amide bond, a sulfonamide bond, or a sulfonate ester bond, and more preferably an ester bond or a sulfonate ester bond. L^{C} is preferably a single bond, an ether bond, an ester bond, an amide bond, or a sulfonate ester bond, and more preferably a single bond, an ether bond, or an ester bond. L^{D} is preferably a single bond, an ether bond, an ester bond, an amide bond, or a sulfonate ester bond, and more preferably a single bond, an ether bond, or an ester bond. L^{E} is preferably a single bond, an ether bond, an ester bond, or a sulfonate ester bond, and more preferably a single bond, an ether bond, or an ester bond.

When m12 is 1, L^{A} and L^{B} are preferably attached to an adjacent carbon atom on an aromatic ring. At this time, since the substituent including the fluorosulfonic acid anion structure and the substituent including an aromatic ring substituted with an iodine atom are present at more spatially close positions, higher sensitivity is expected to be achieved.

In the formula (Z), X^{L1} and X^{L2} are each independently a single bond or a C₁-C₄₀ hydrocarbylene group which may contain a heteroatom. The hydrocarbylene group may be straight, branched, or cyclic, and specific examples thereof include an alkanediyl group, a cyclic saturated hydrocarbylene group, and an arylene group. Specific examples of the heteroatom include an oxygen atom, a nitrogen atom, and a sulfur atom.

Specific examples of the C₁-C₄₀ hydrocarbylene group which may contain a heteroatom of X^{L1} and X^{L2} include those shown below, but are not limited thereto. In the following formulae, * each designates a point of attachment to L^{A} and L^{C}, or L^{B} and L^{D}.

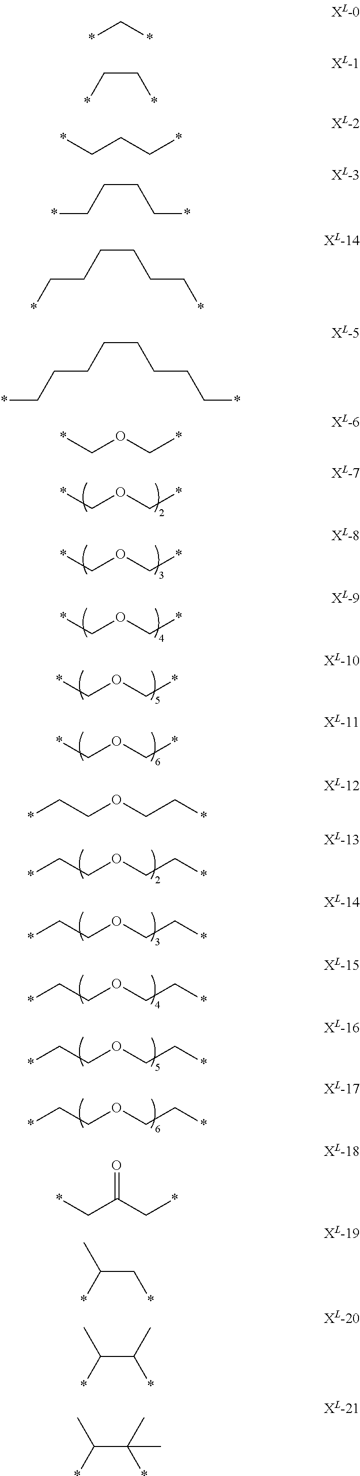

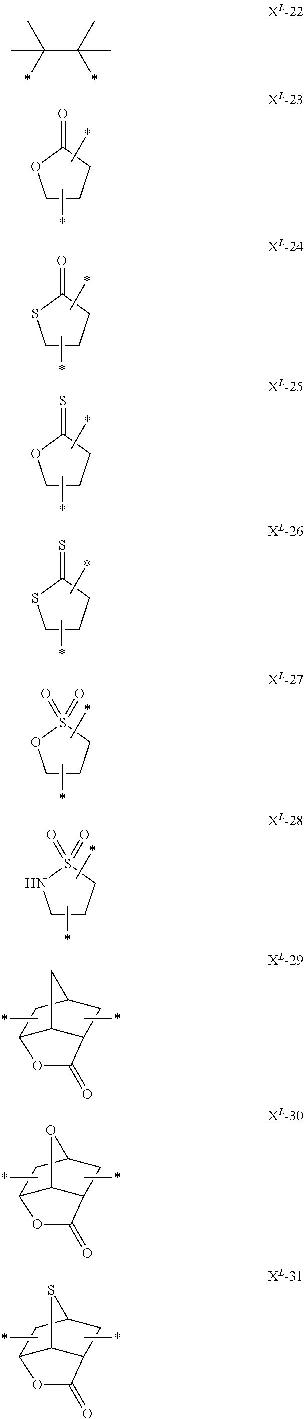

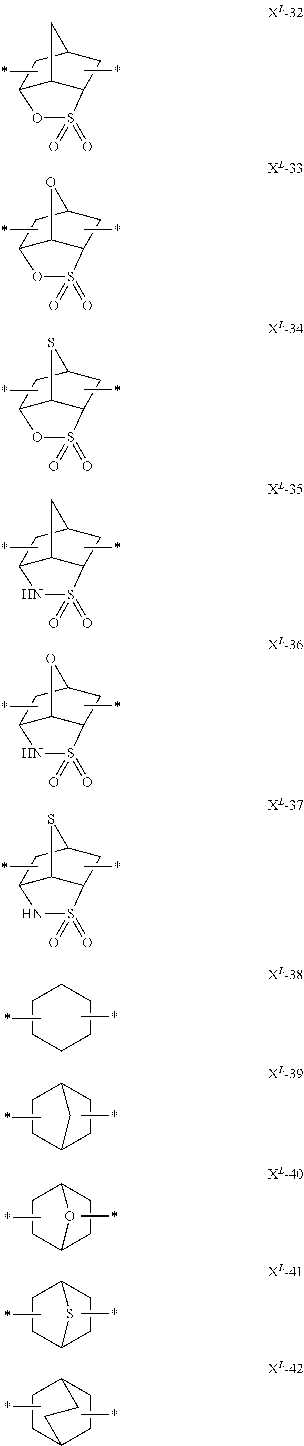

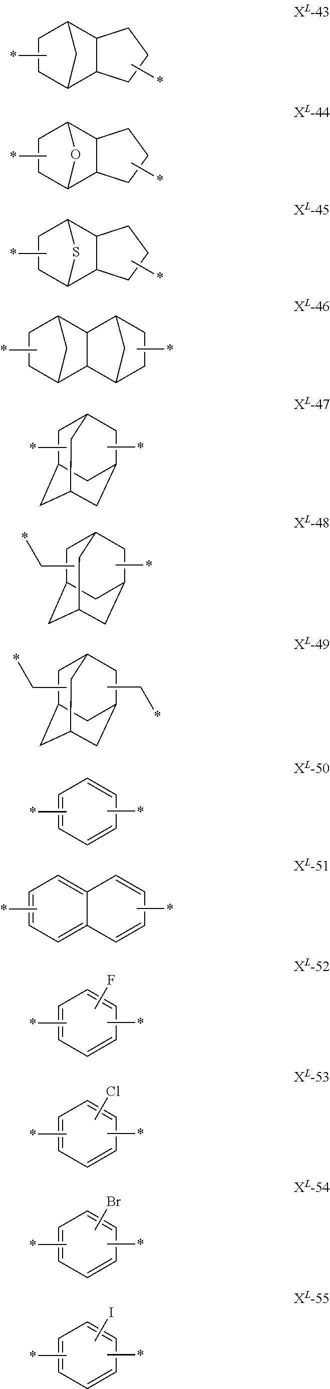

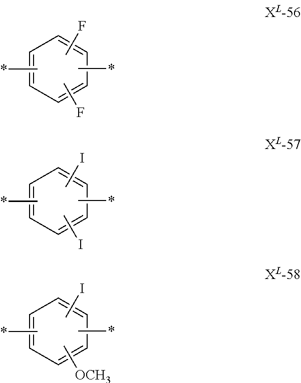

Among them, X^{L}—O to X^{L}-22, X^{L}-29 to X^{L}-34, and X^{L}-47 to X^{L}-58 are preferred.

In the formula (Z), Q¹ and Q² are each independently a hydrogen atom, a fluorine atom, or a C₁-C₆ fluorinated saturated hydrocarbyl group. The C₁-C₆ fluorinated saturated hydrocarbyl group is preferably a trifluoromethyl group.

In the formula (Z), Q³ and Q⁴ are each independently a fluorine atom or a C₁-C₆ fluorinated saturated hydrocarbyl group. The C₁-C₆ fluorinated saturated hydrocarbyl group is preferably a trifluoromethyl group. Q³ and Q⁴ are more preferably fluorine atoms.

Specific examples of the partial structure of —[C(Q¹)(Q²)]ₙ₆—C(Q³)(Q⁴)—SO₃⁻in the formula (Z) are preferably those shown below, but are not limited thereto. In the following formulae, * designates a point of attachment to L^{E}.

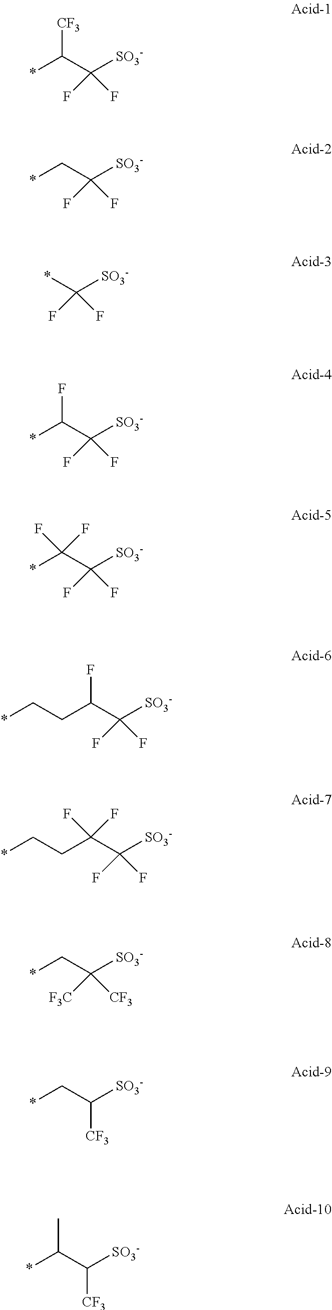

Among them, Acid-1 to Acid-7 are preferred, and Acid-1 to Acid-3, Acid-6, and Acid-7 are more preferred.

Examples of the anion of the sulfonium salt monomer having the formula (1) include those shown below, but are not limited thereto. In the following formulae, R^{A} and Q¹ are as defined above, and Me is a methyl group. The positions of attachment of substituents on the aromatic ring are interchangeable.

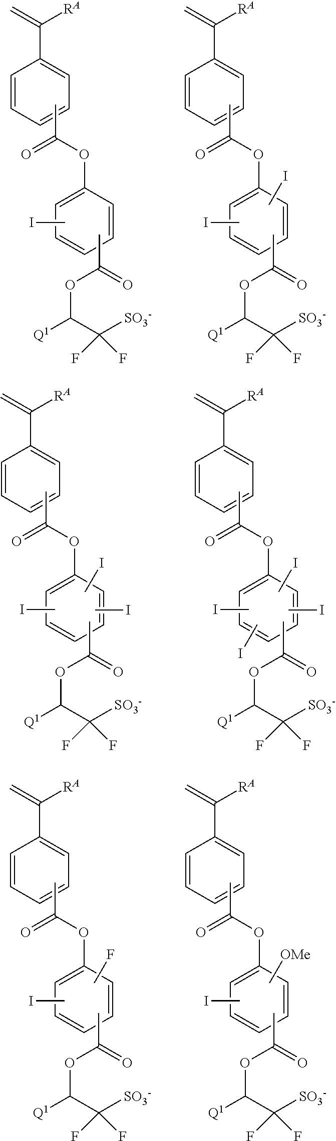

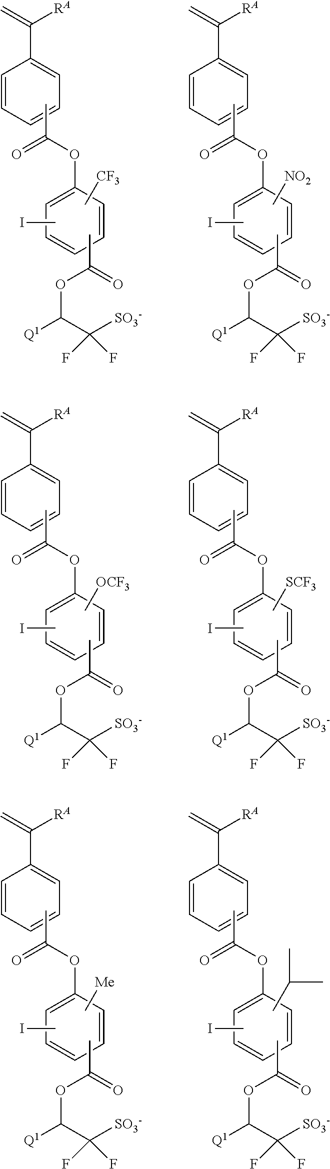

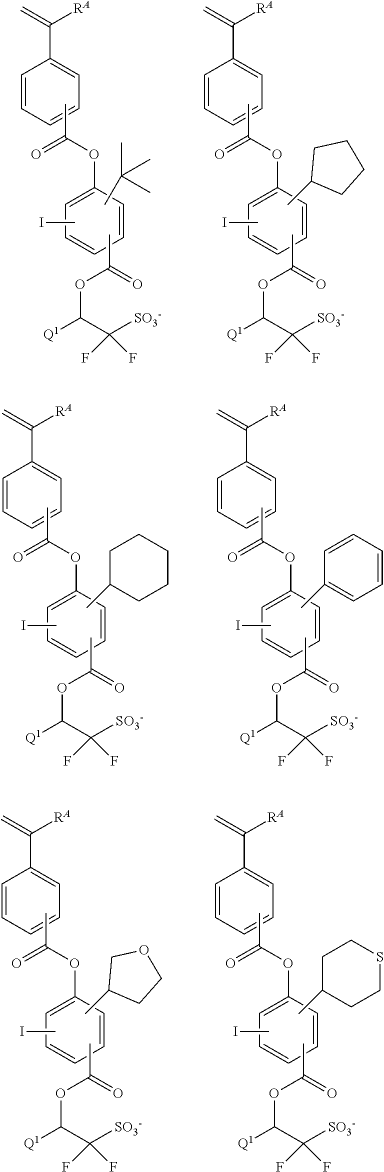

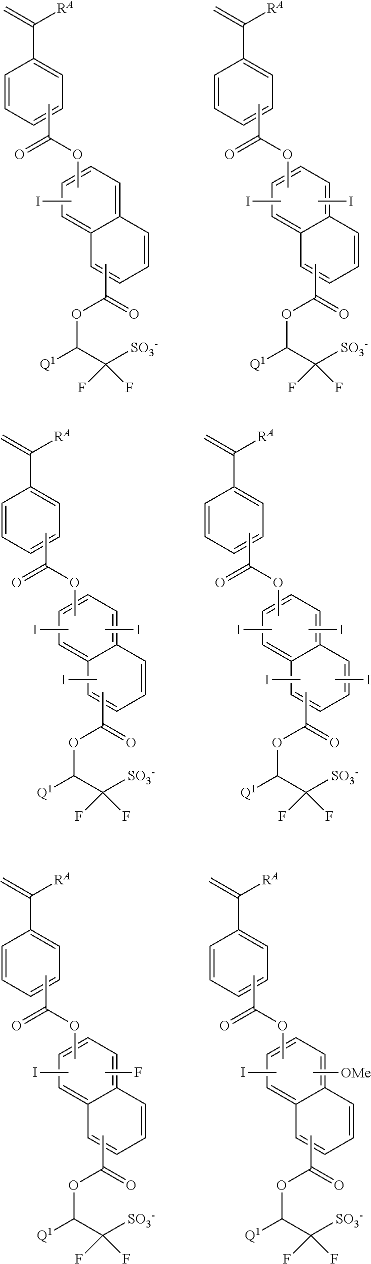

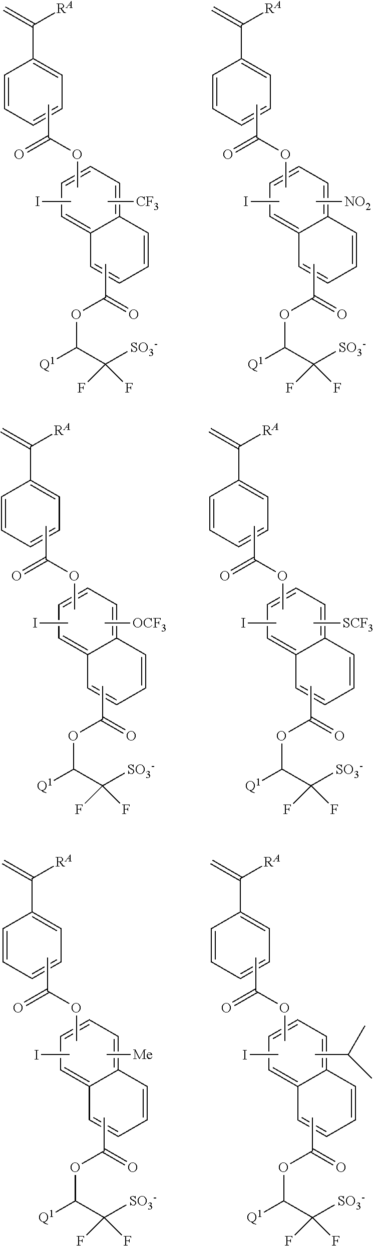

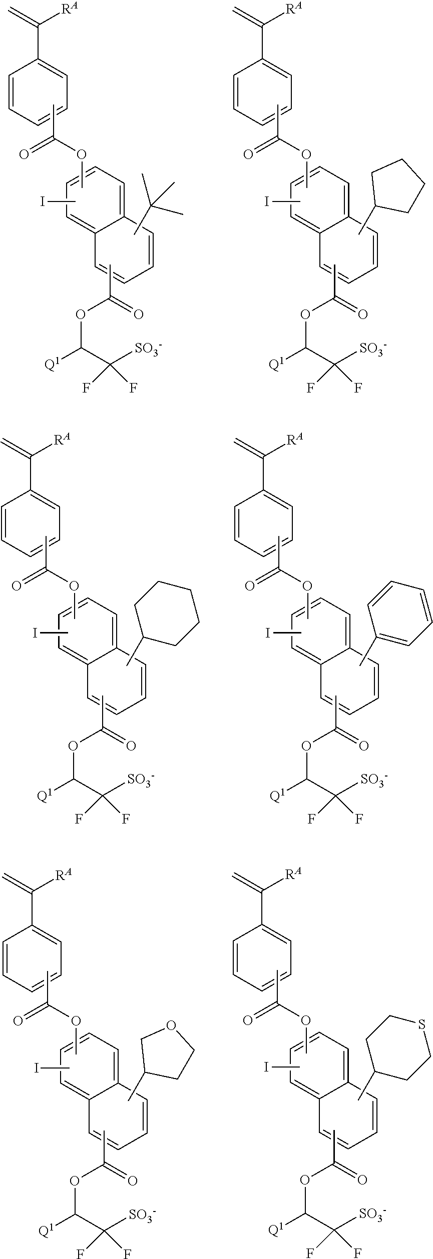

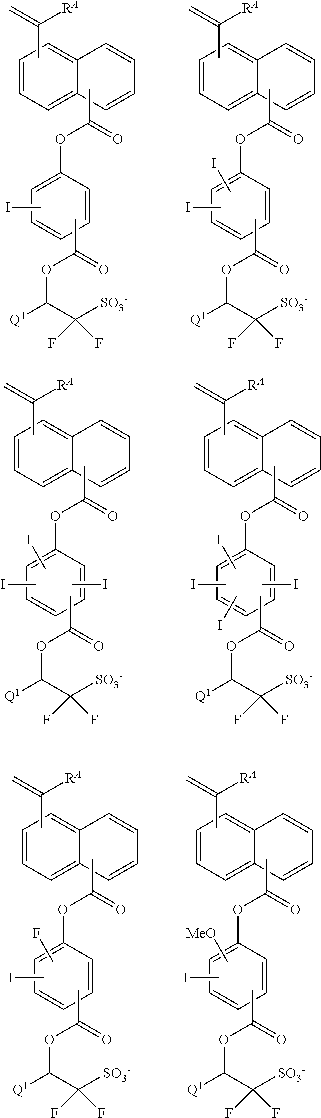

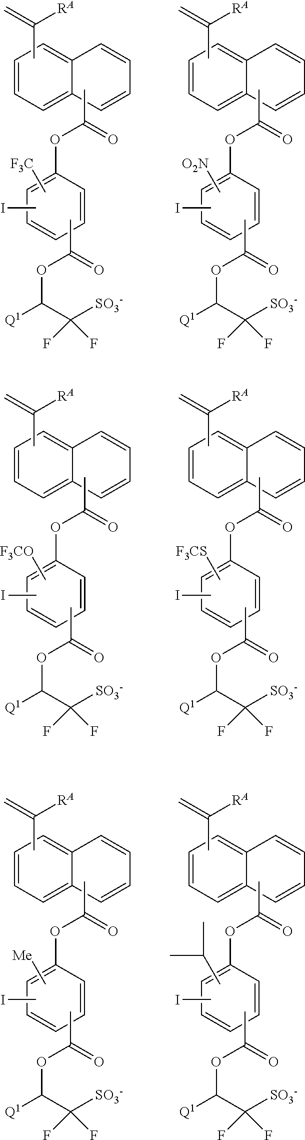

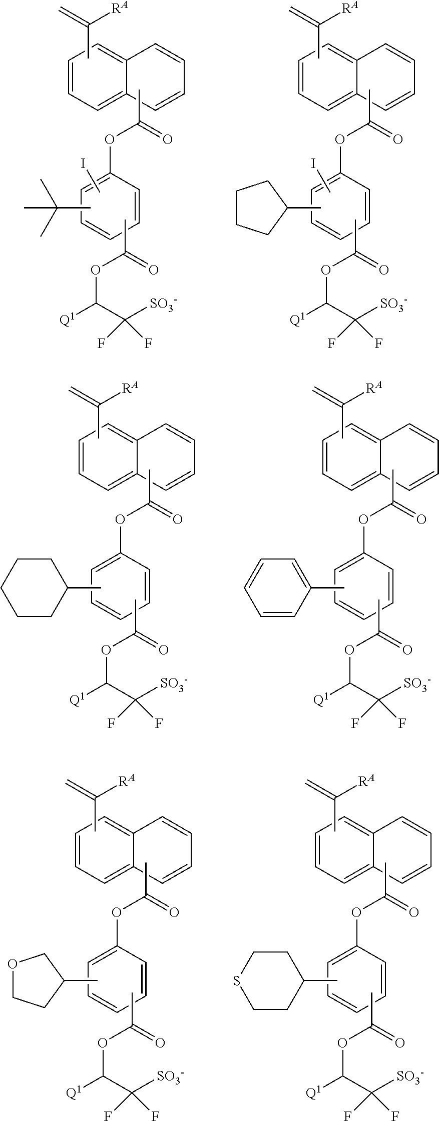

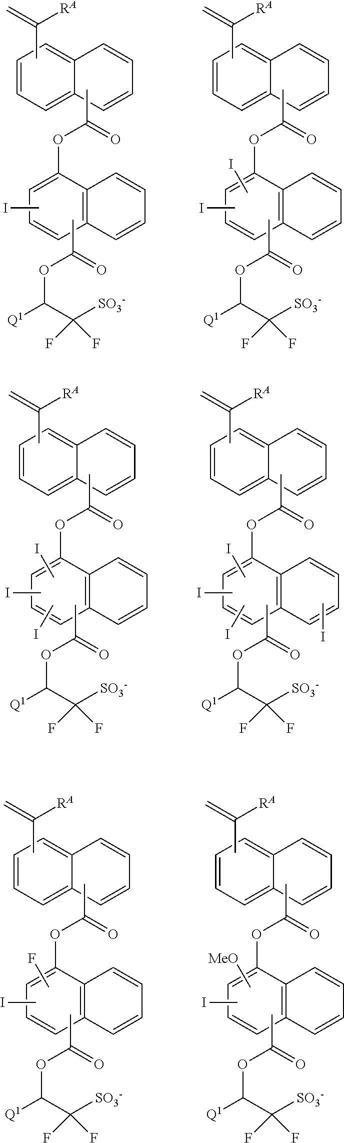

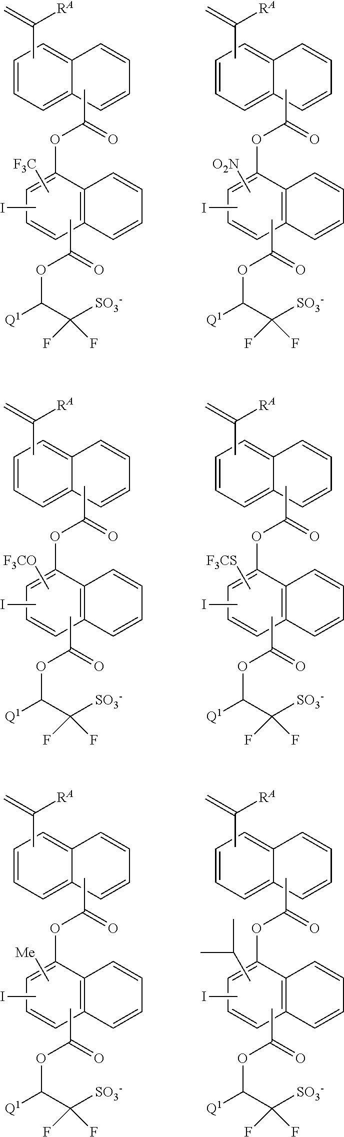

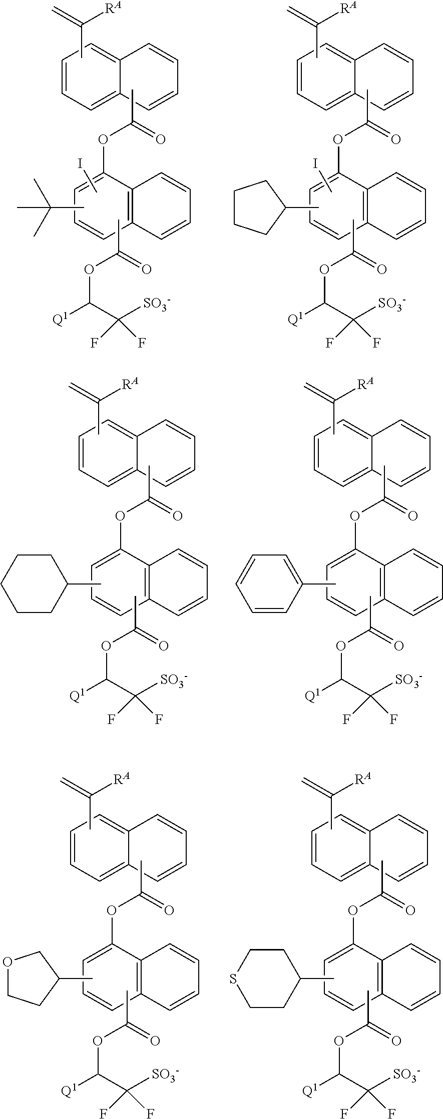

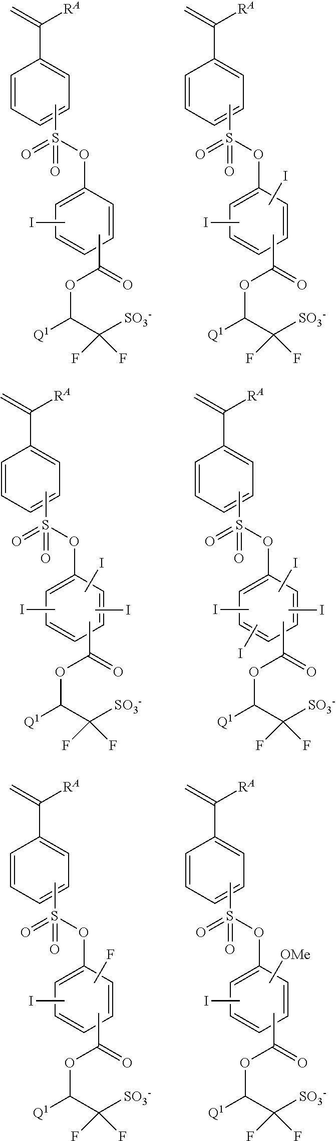

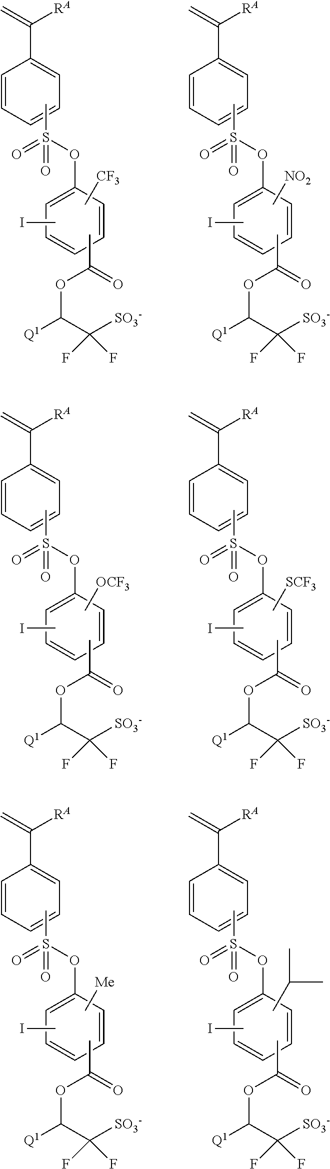

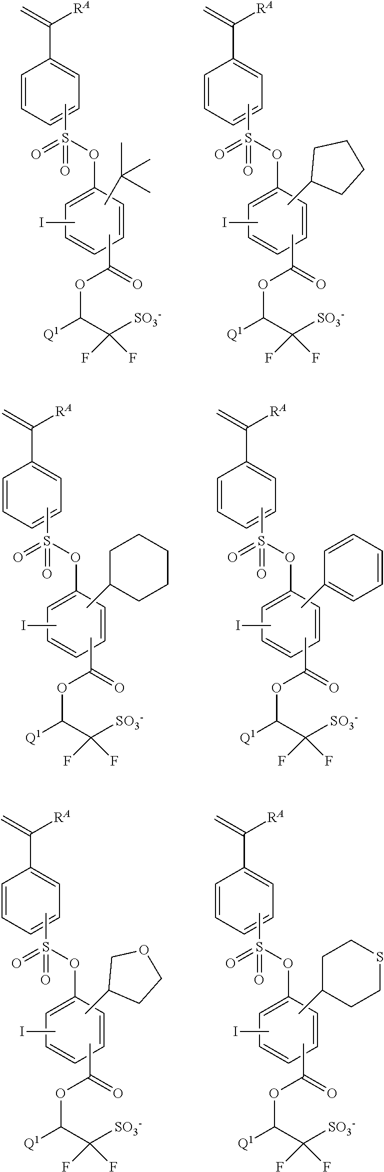

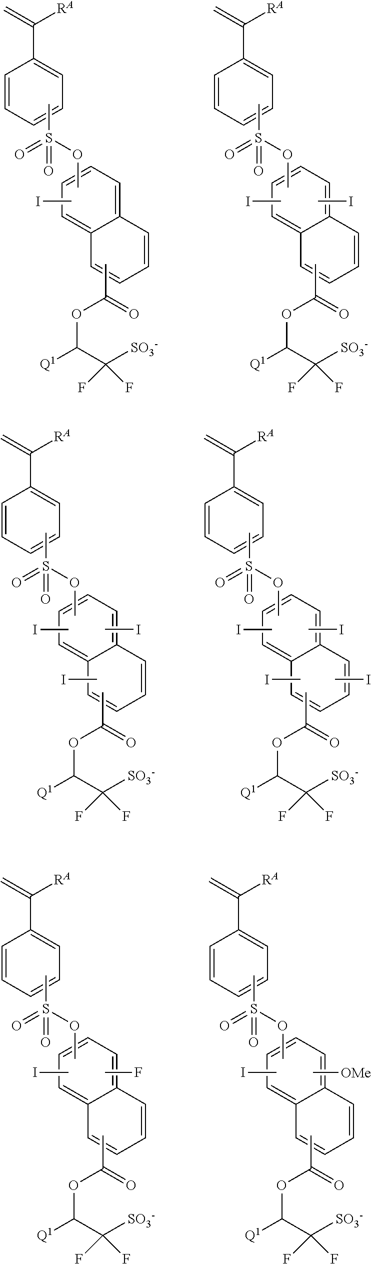

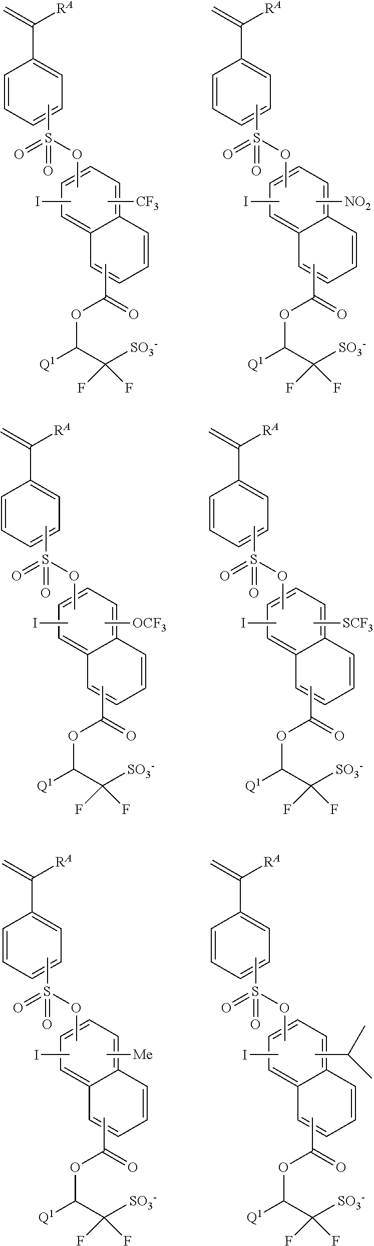

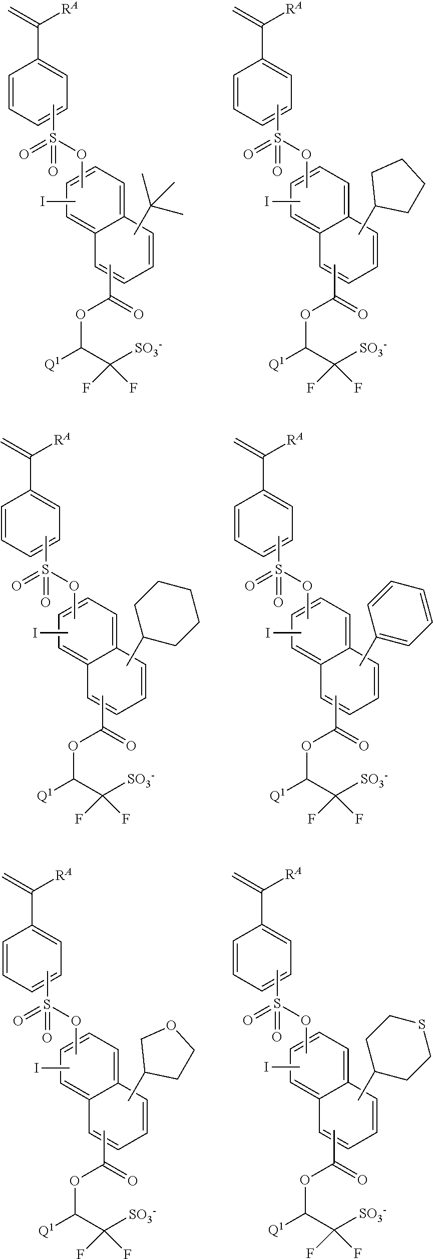

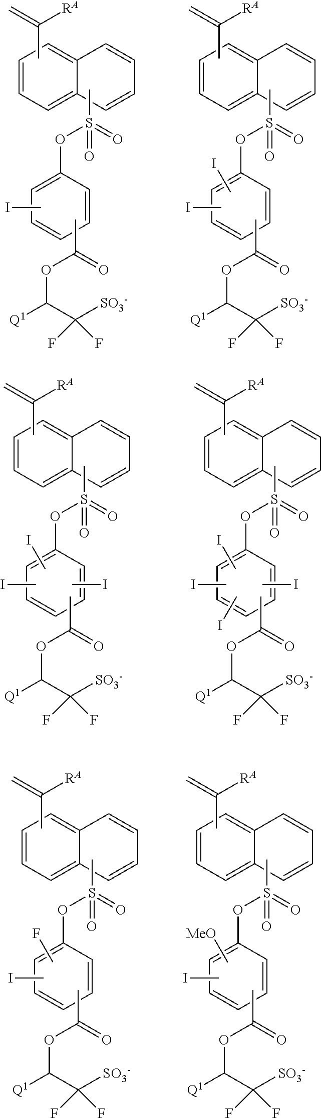

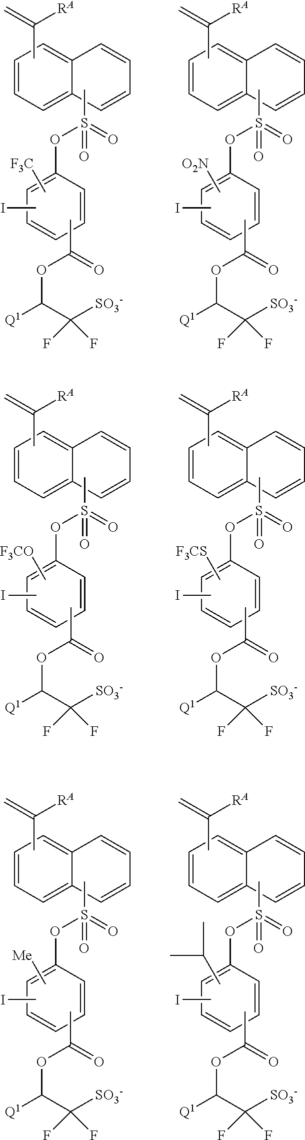

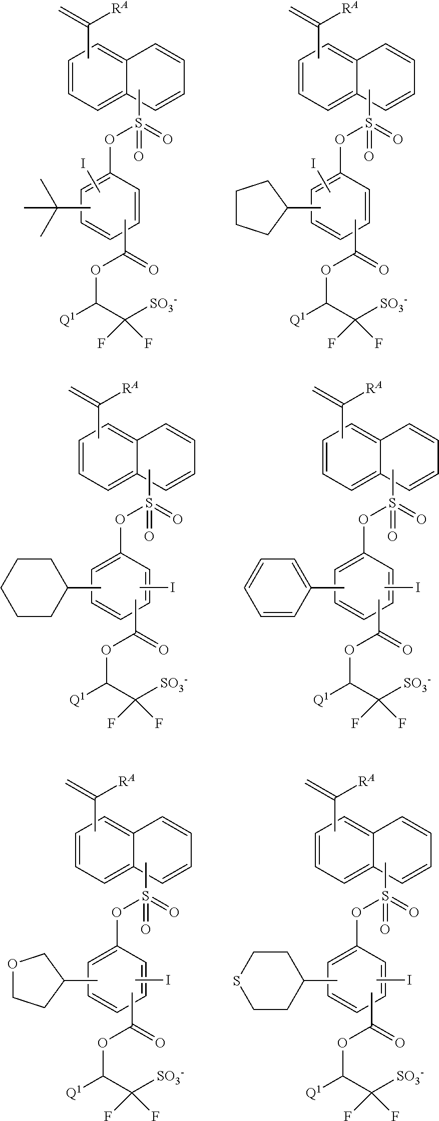

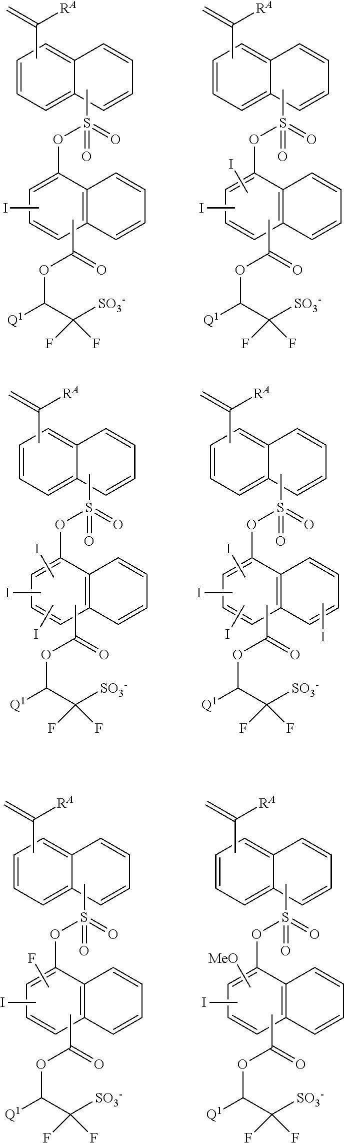

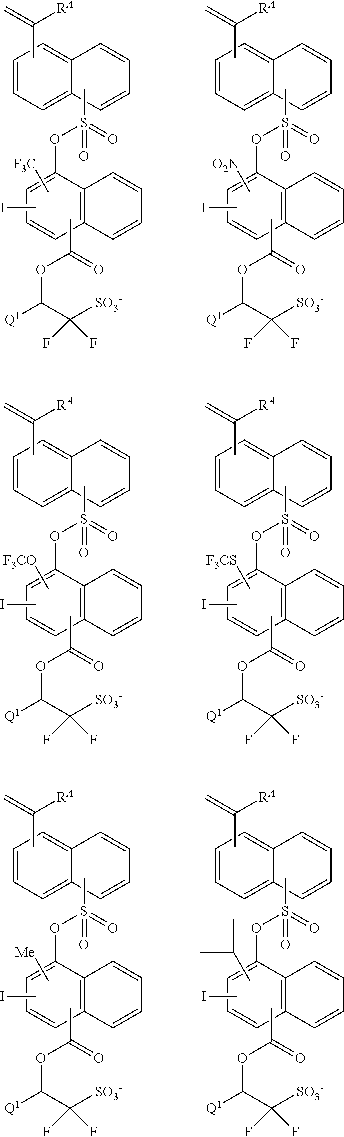

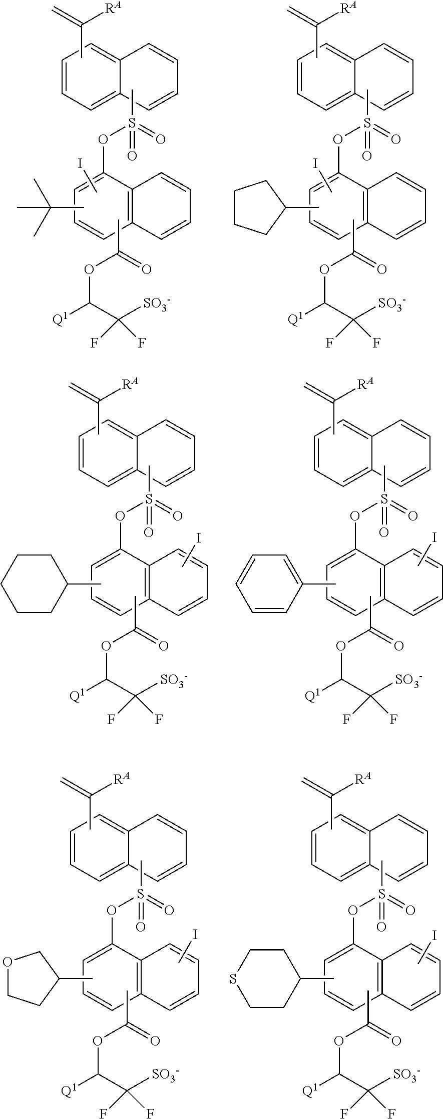

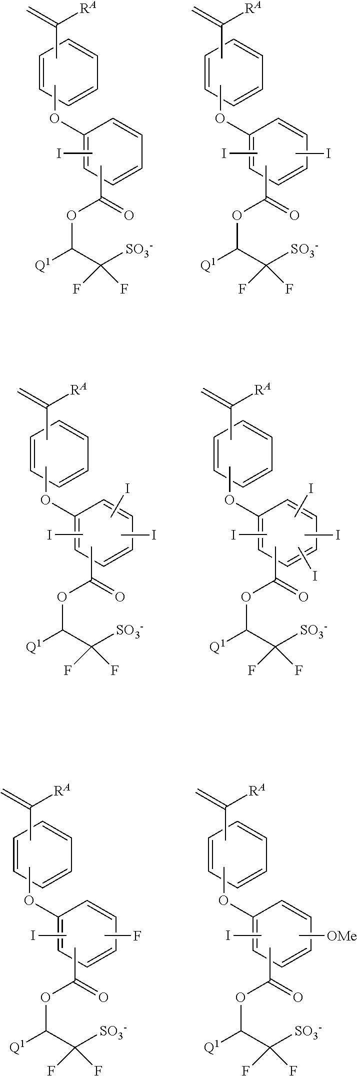

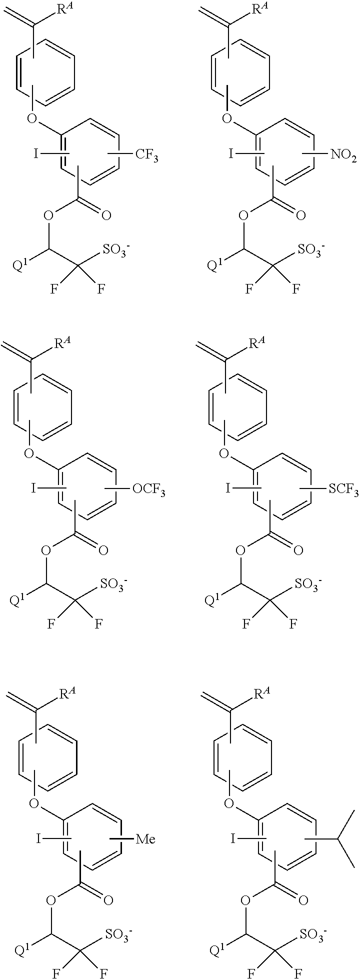

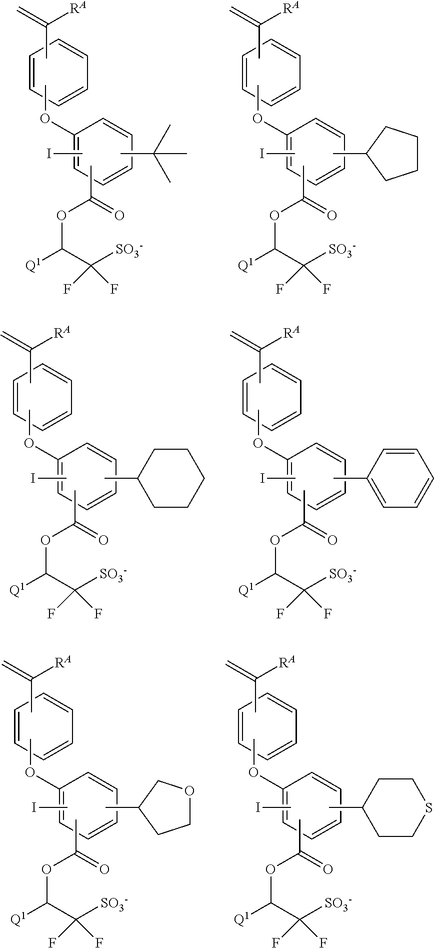

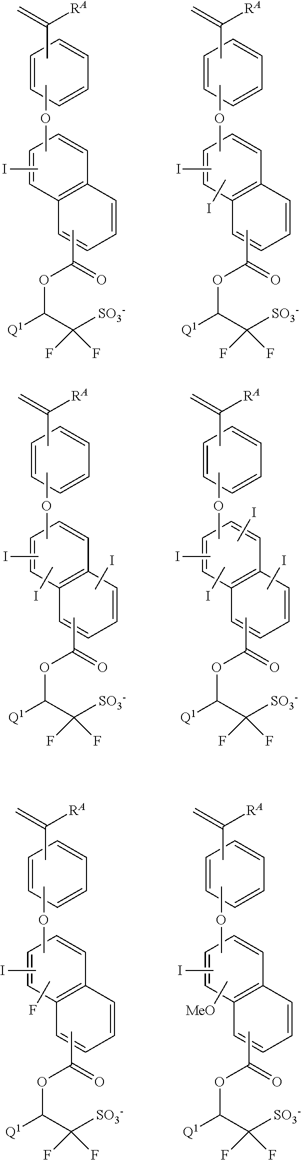

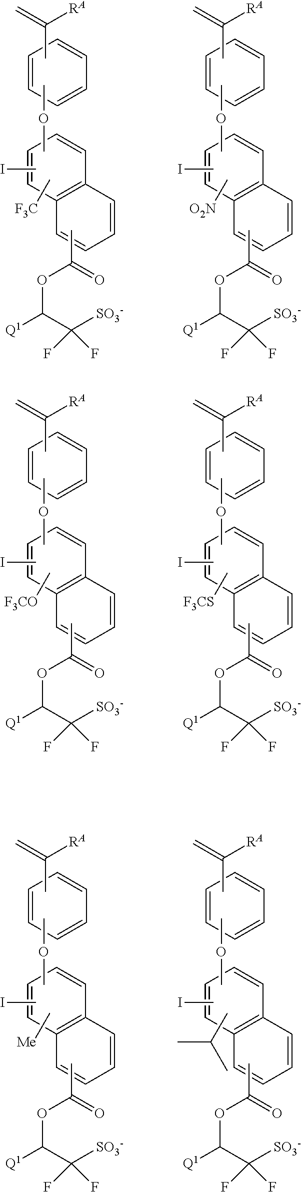

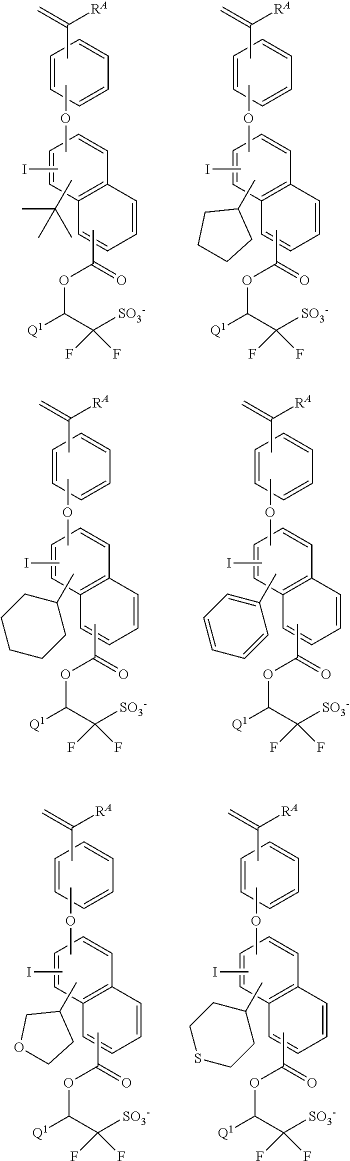

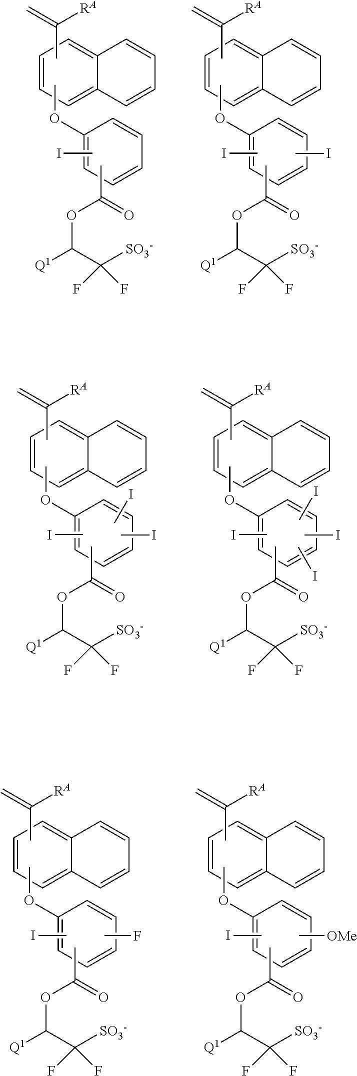

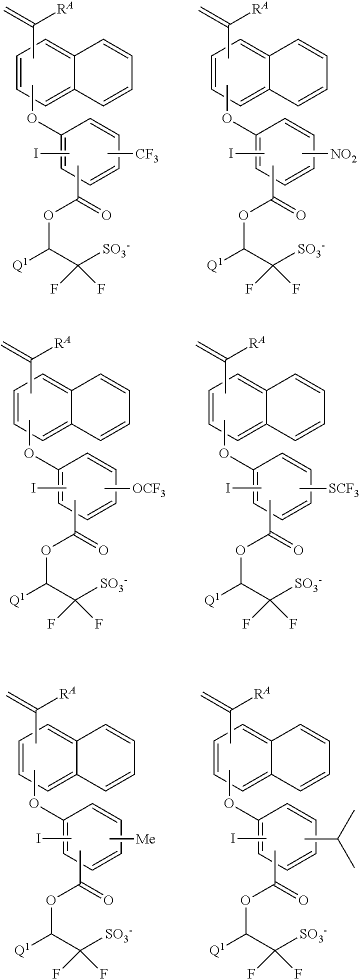

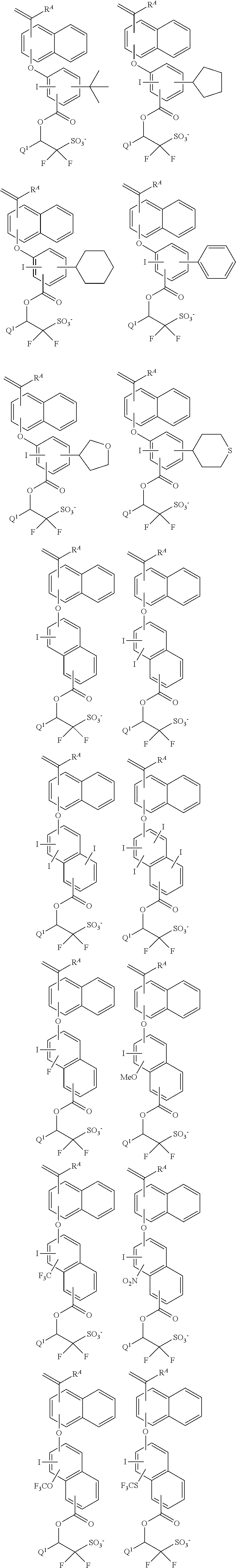

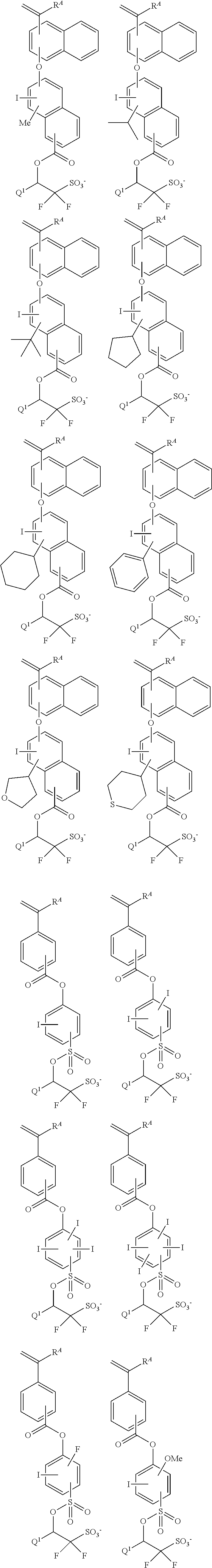

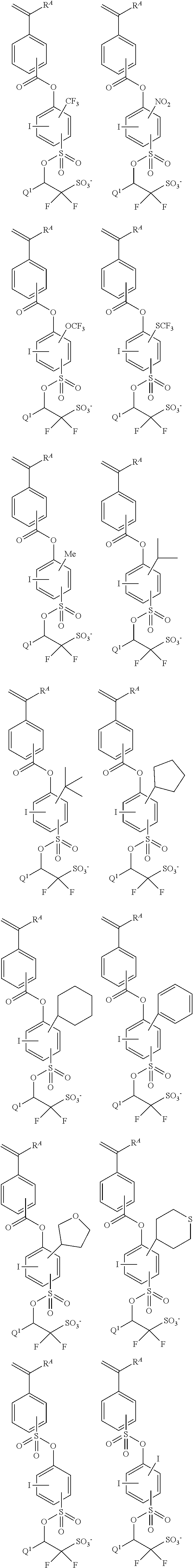

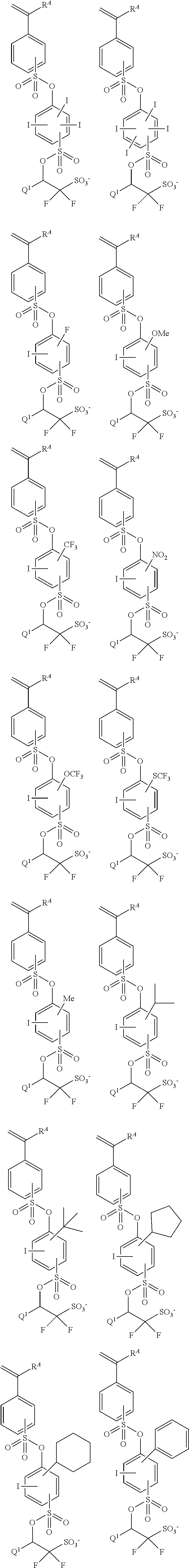

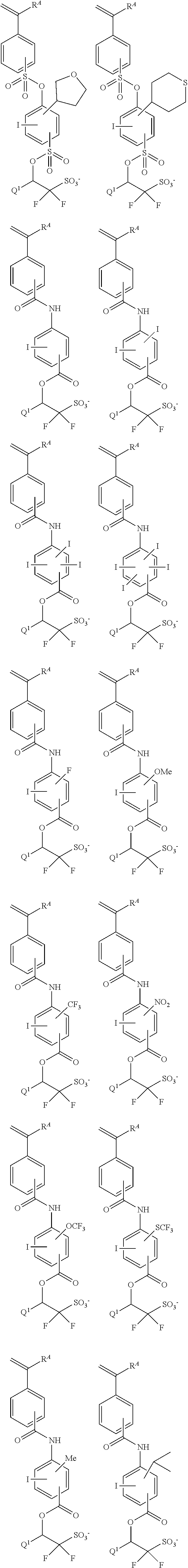

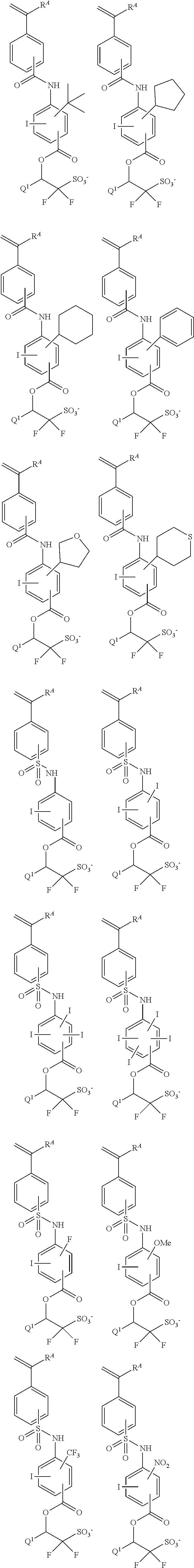

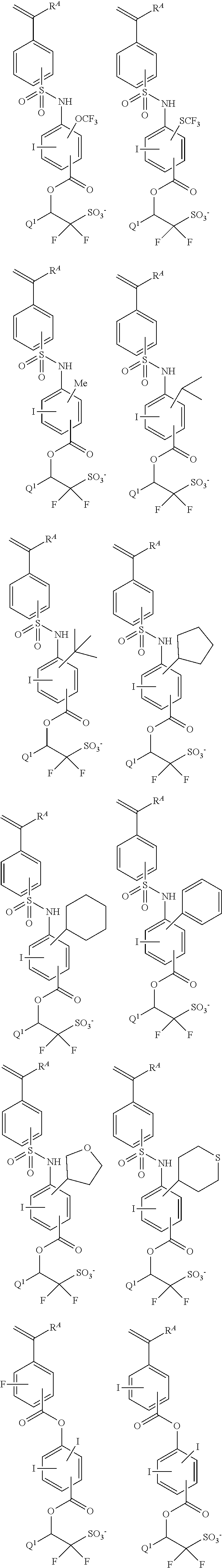

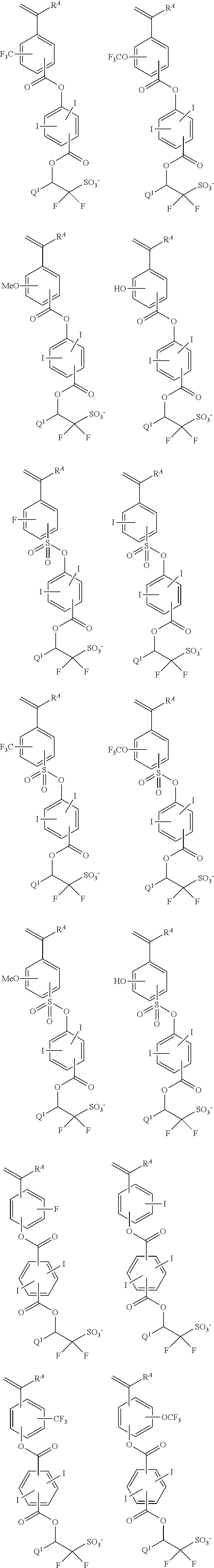

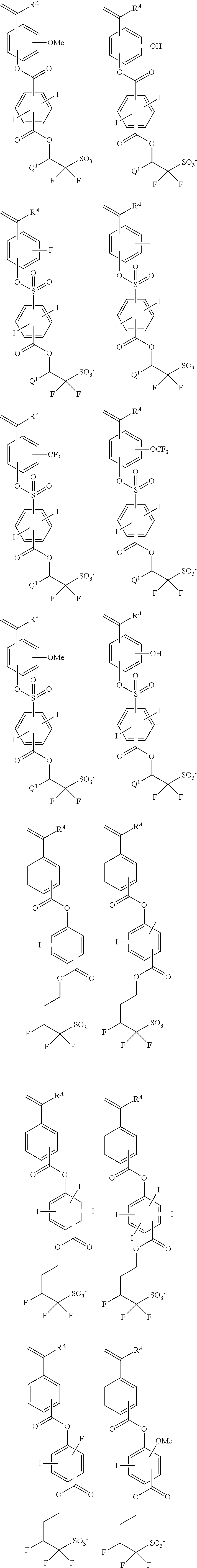

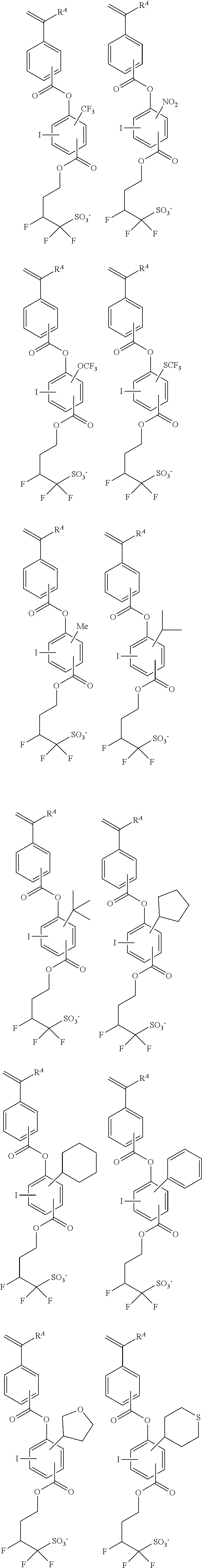

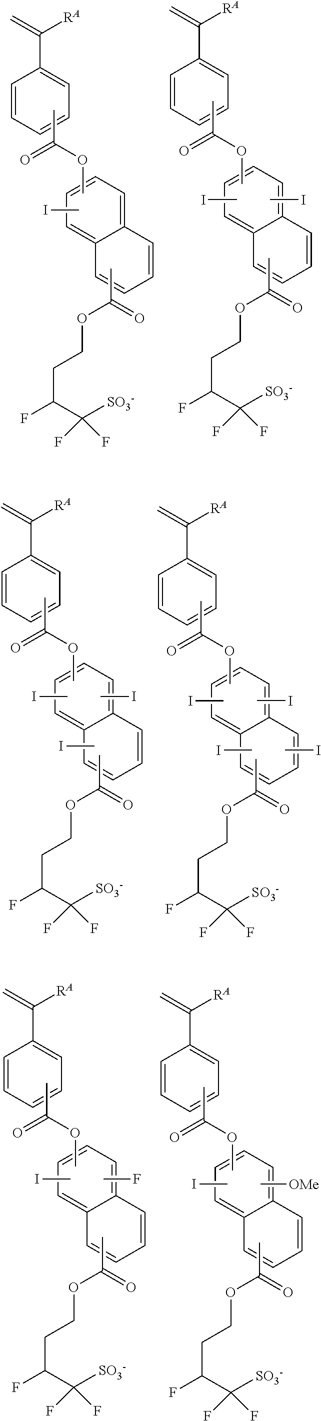

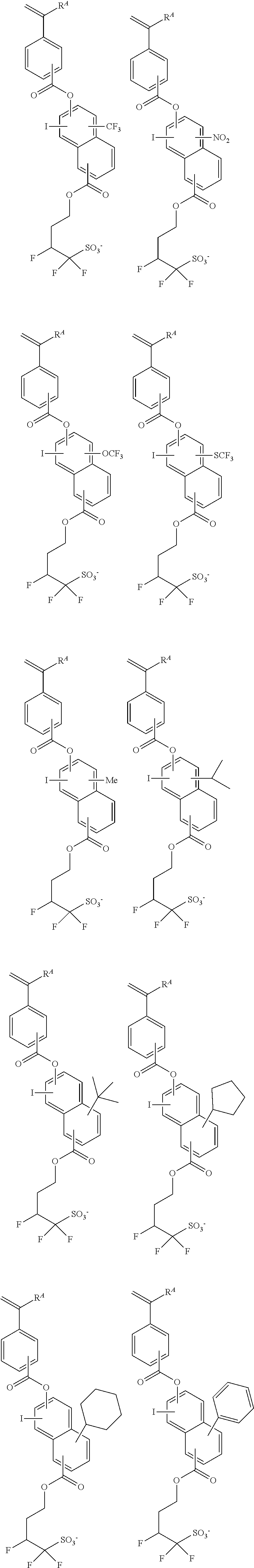

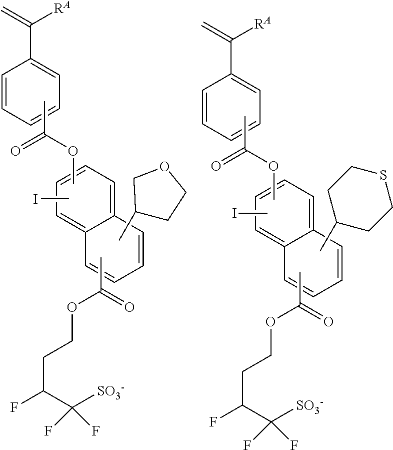

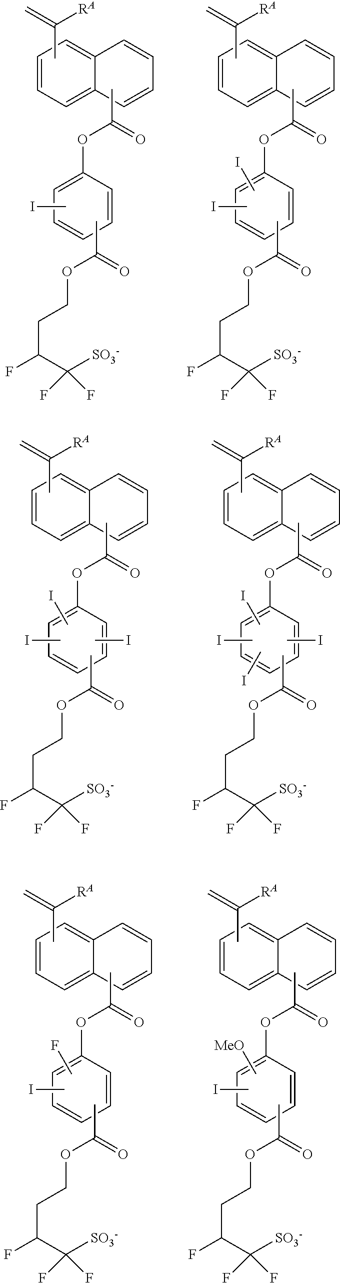

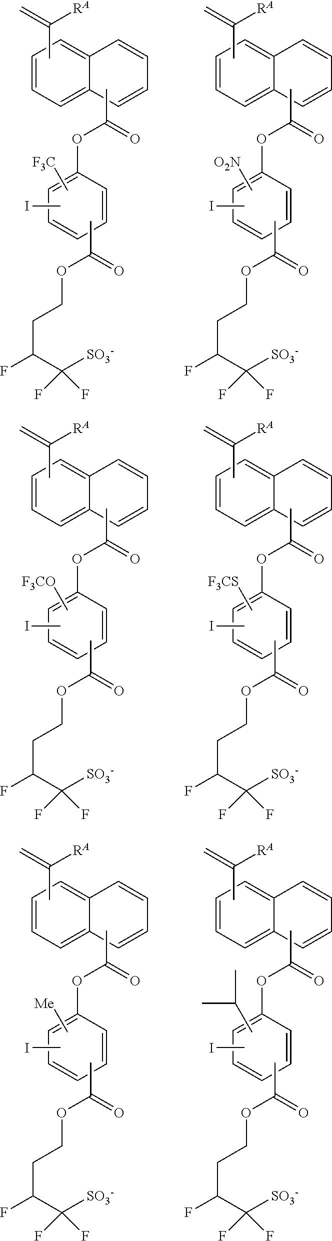

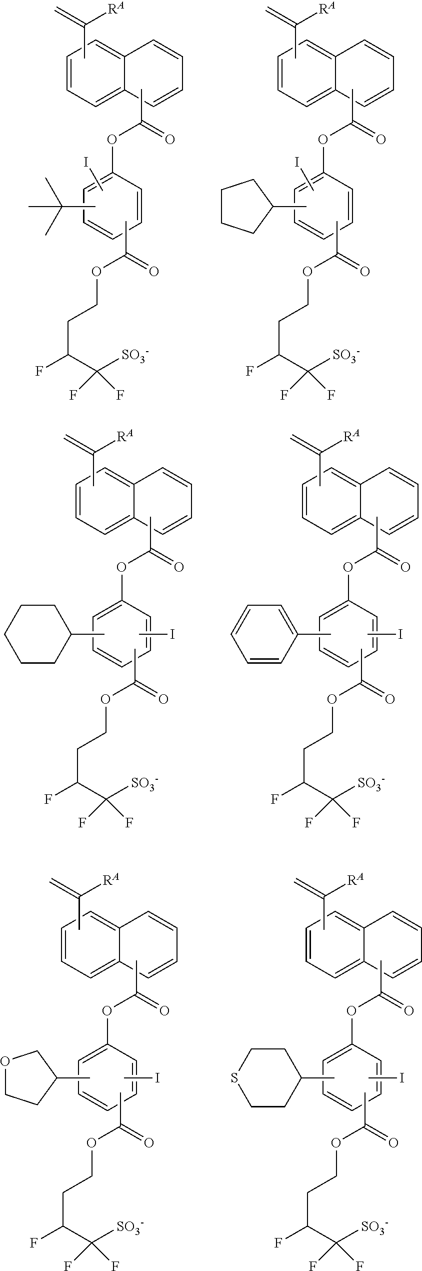

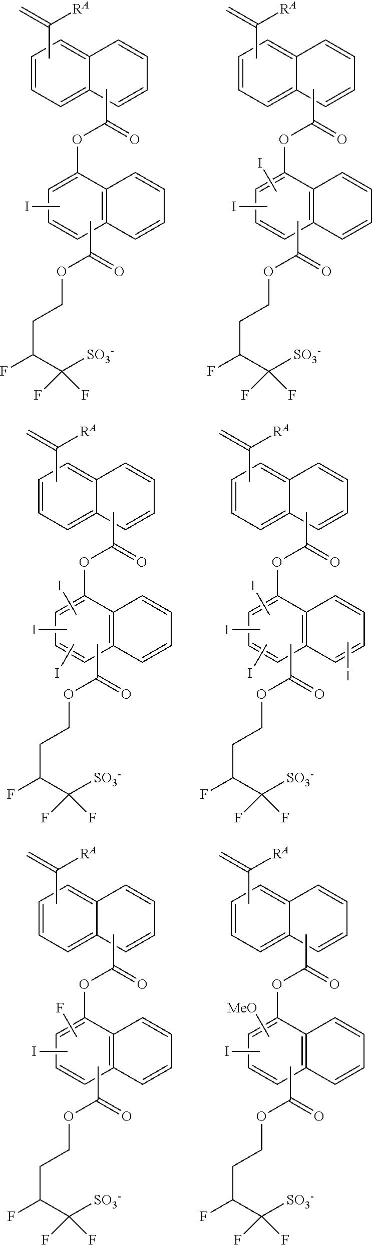

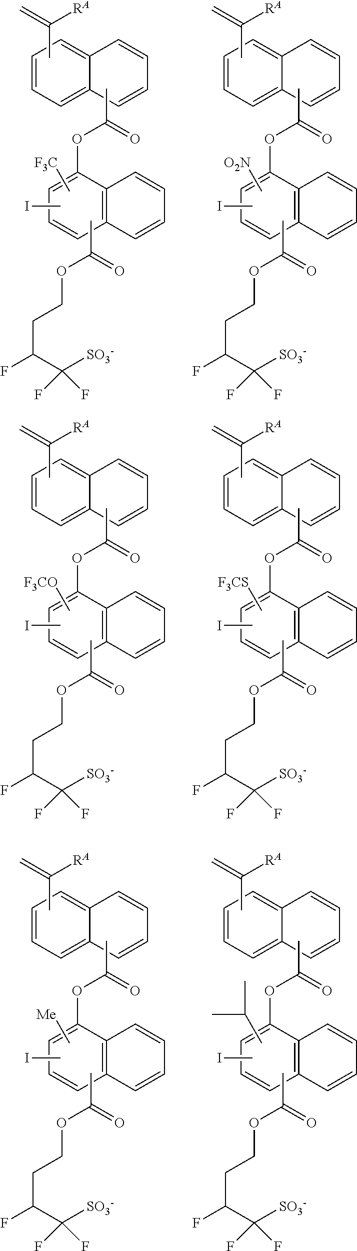

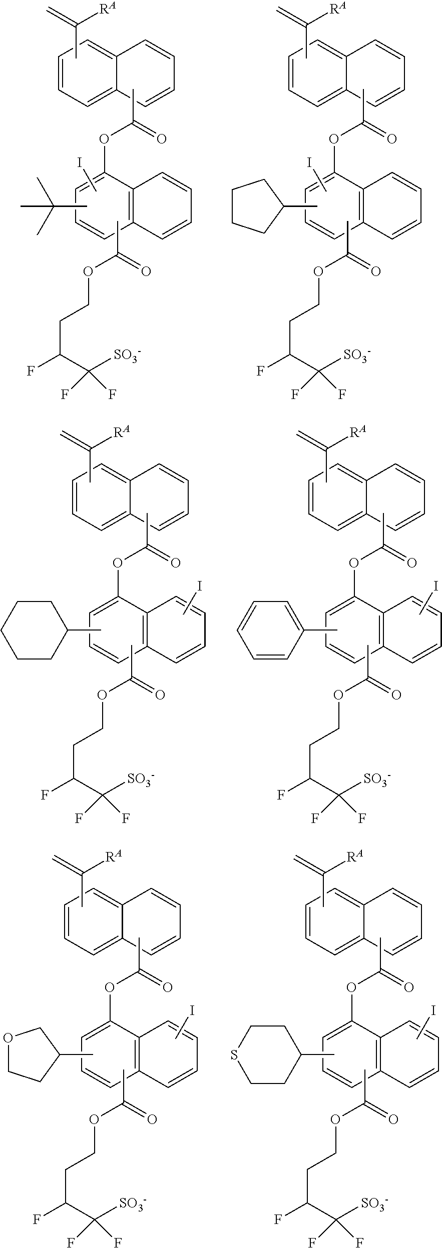

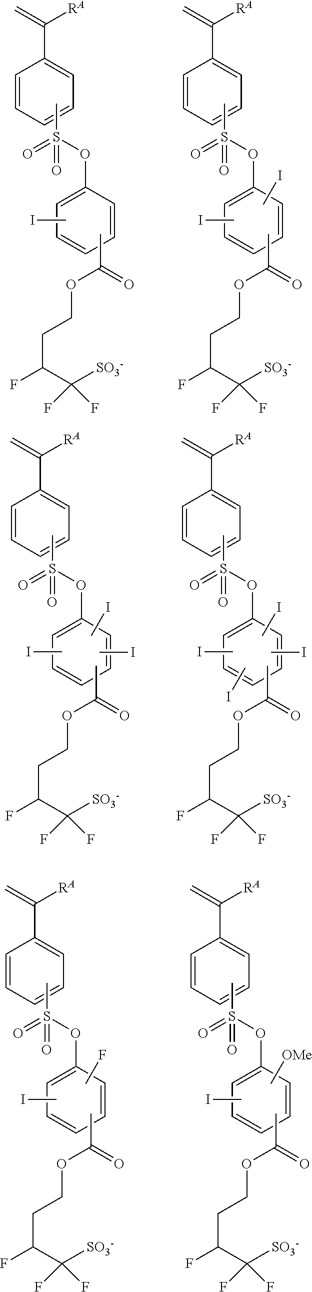

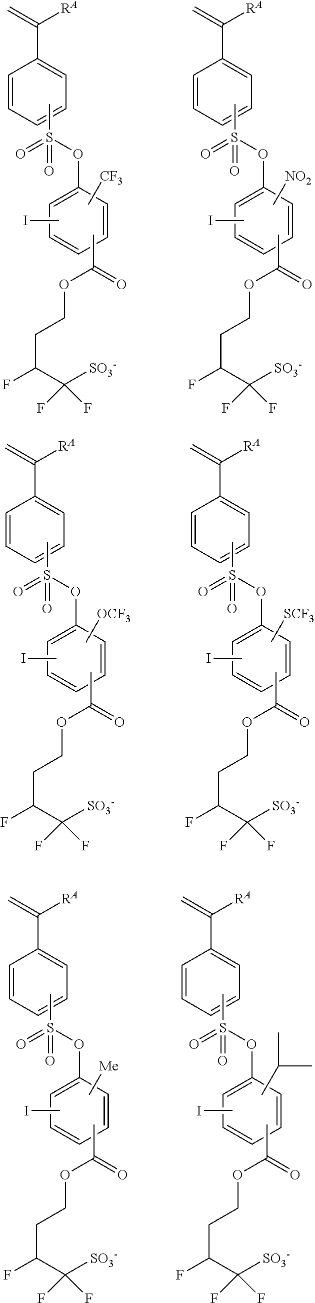

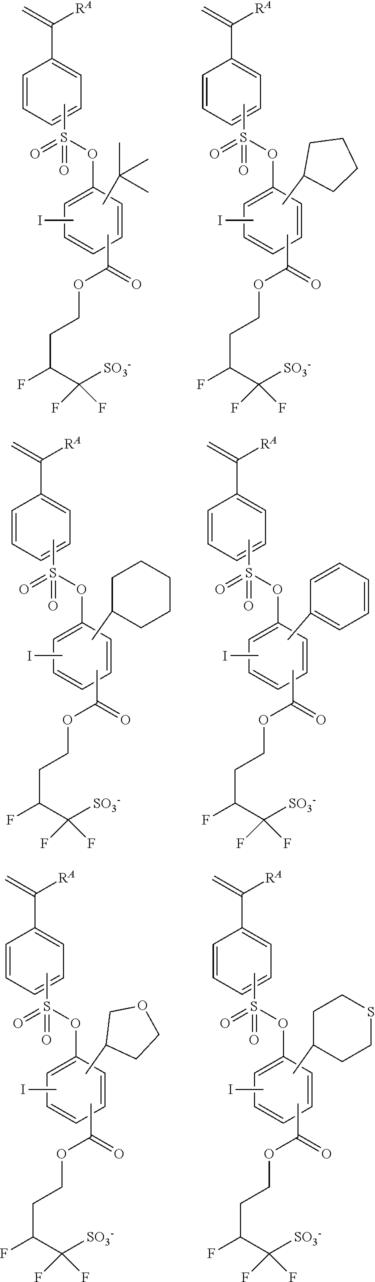

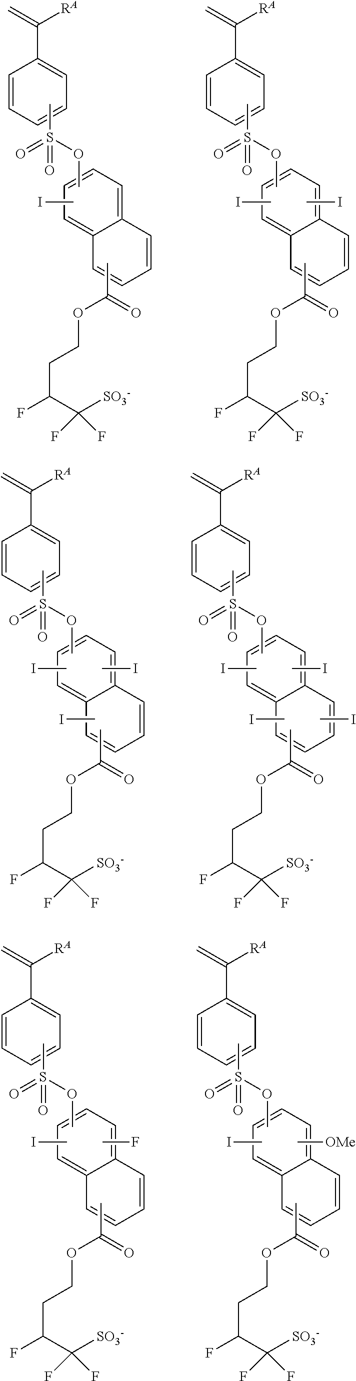

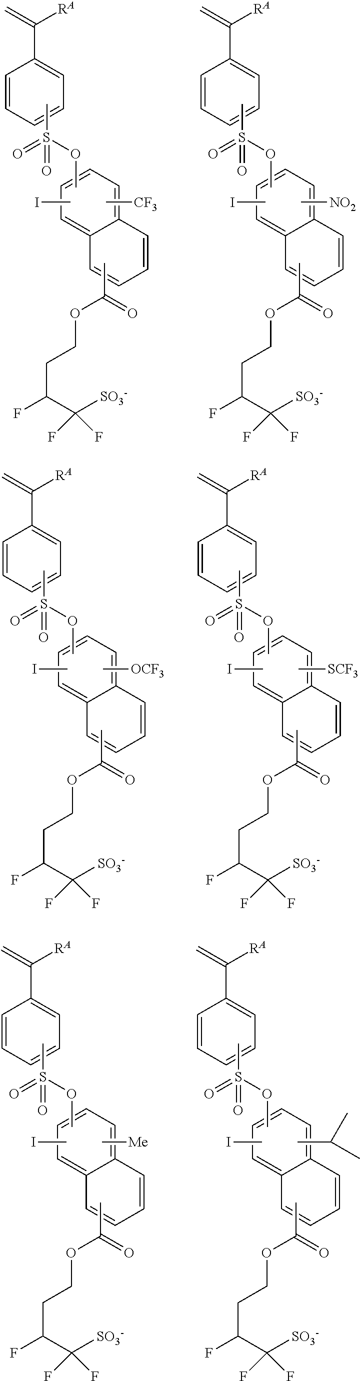

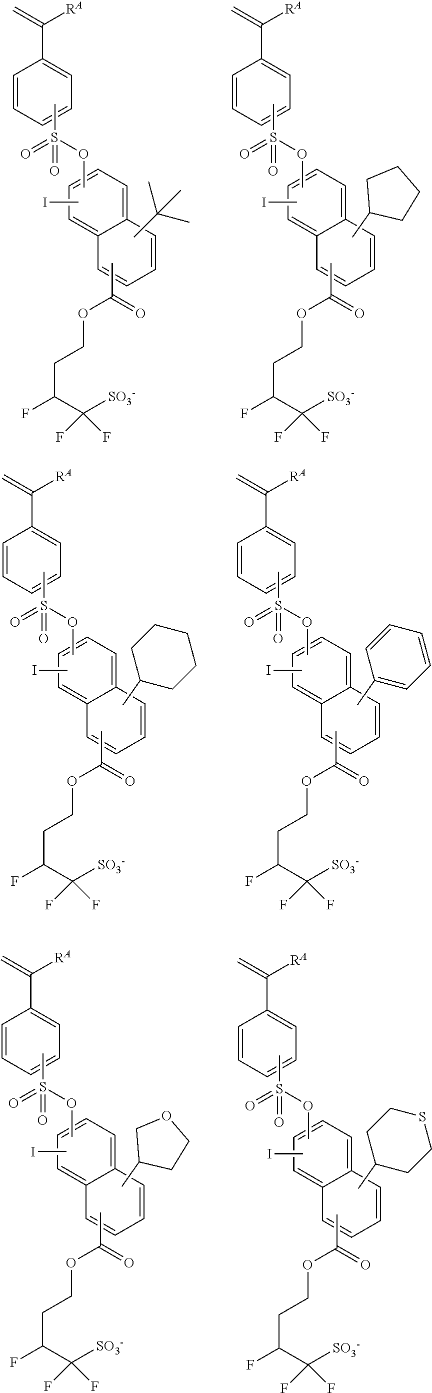

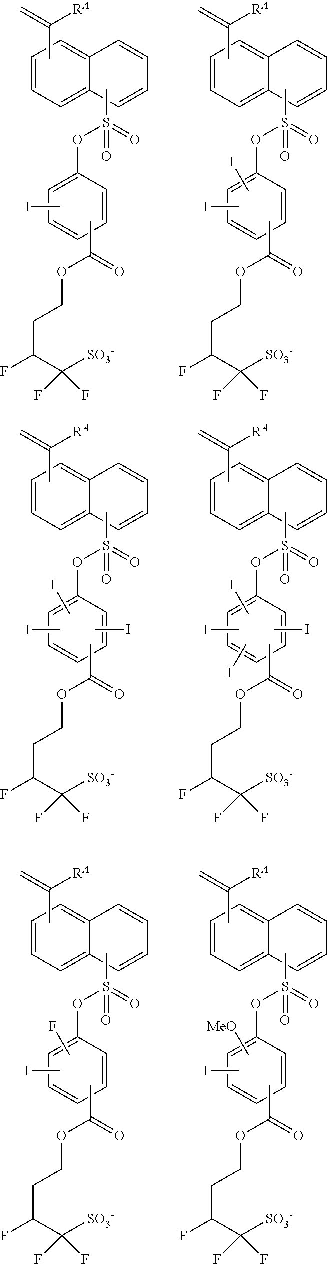

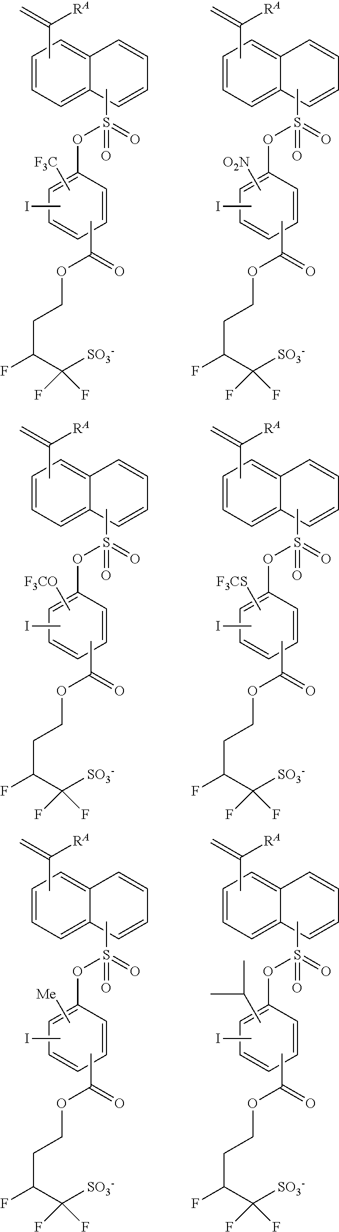

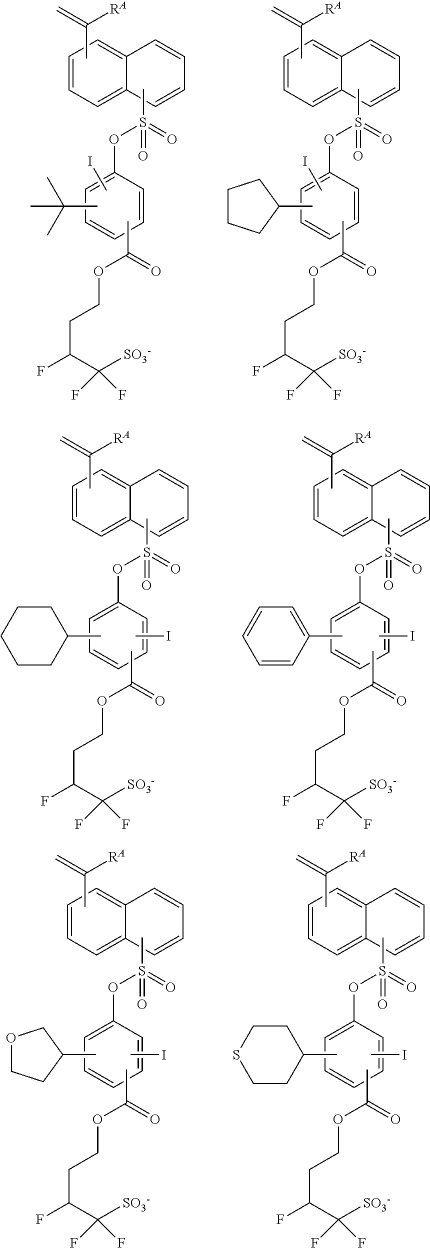

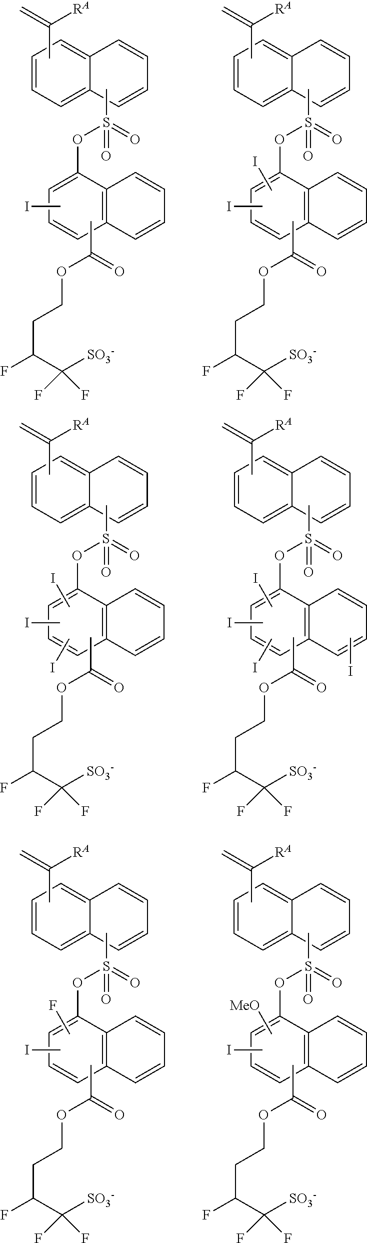

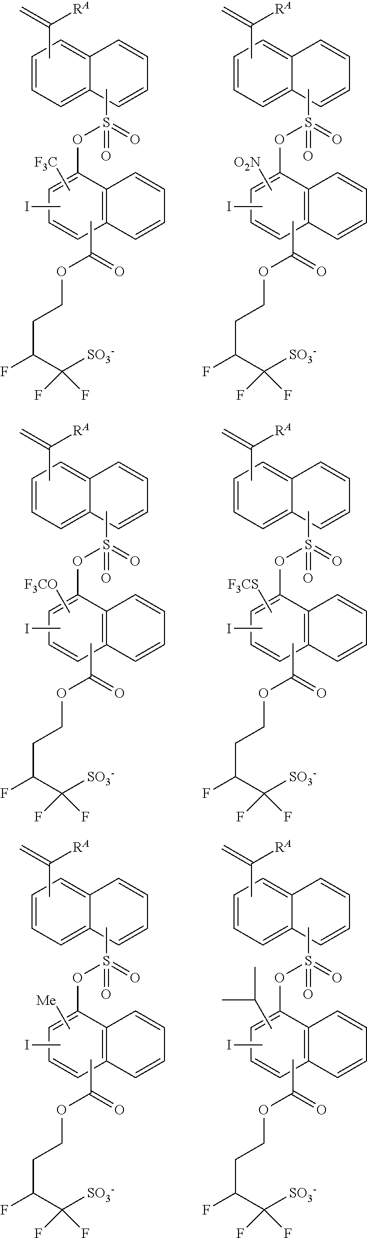

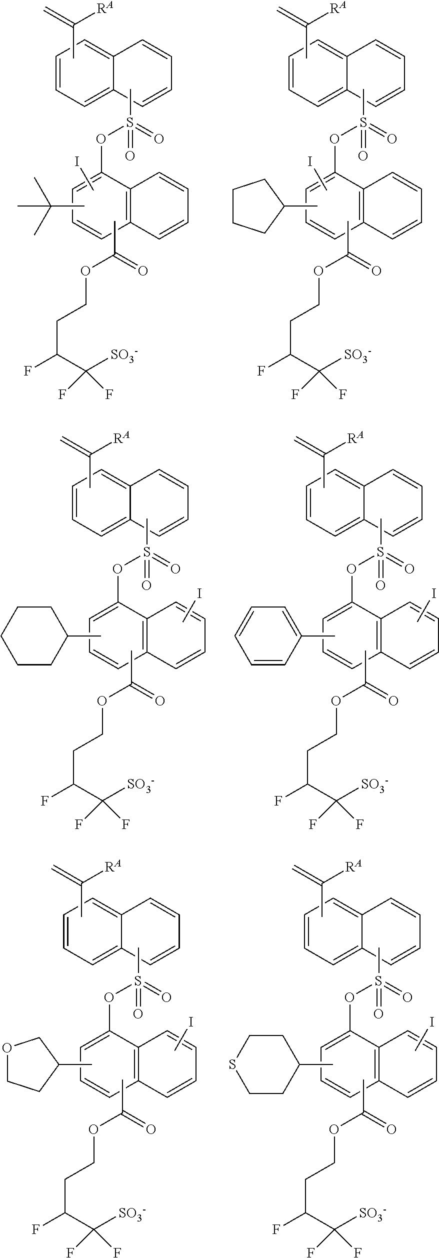

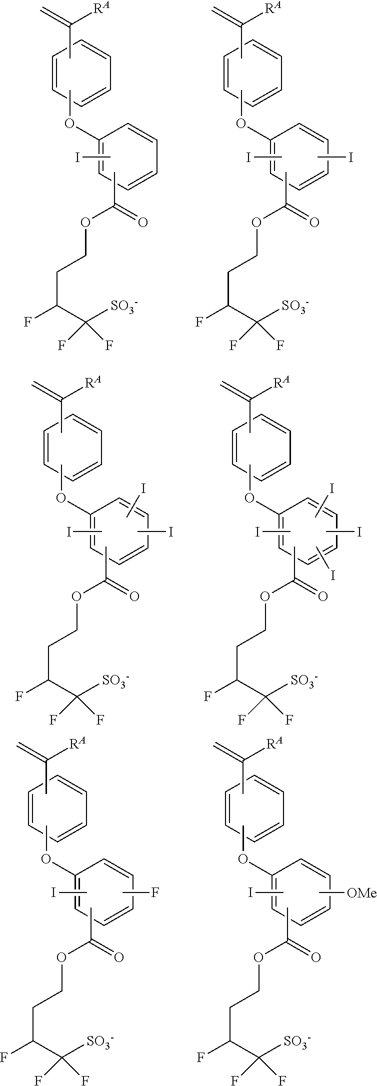

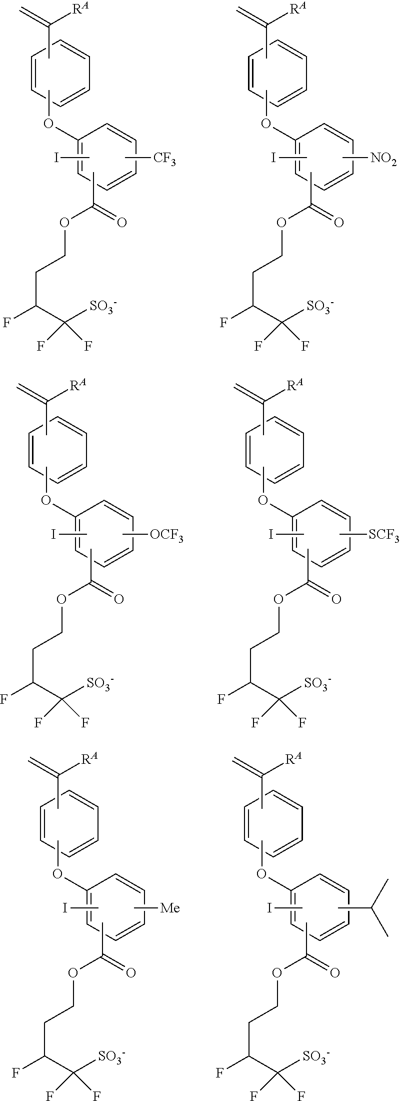

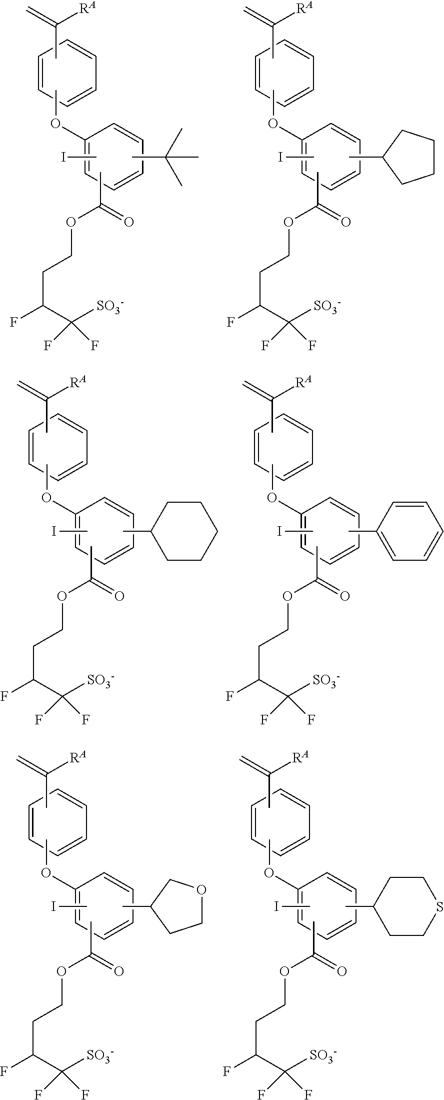

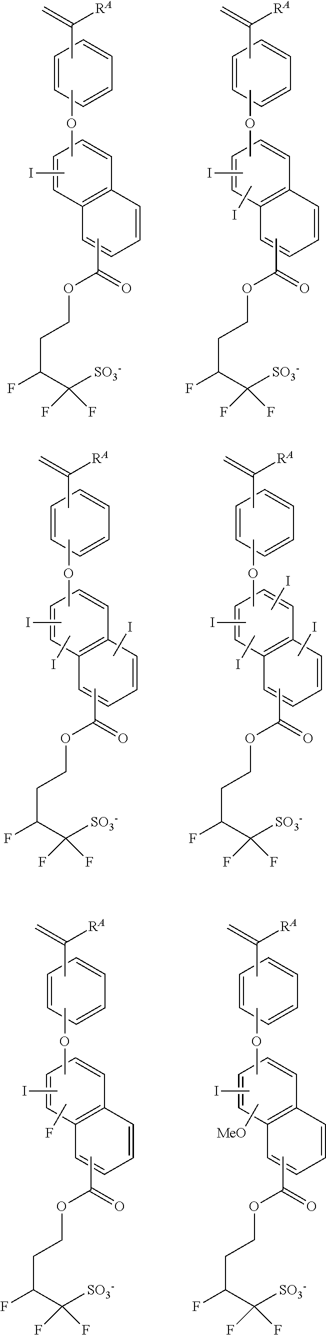

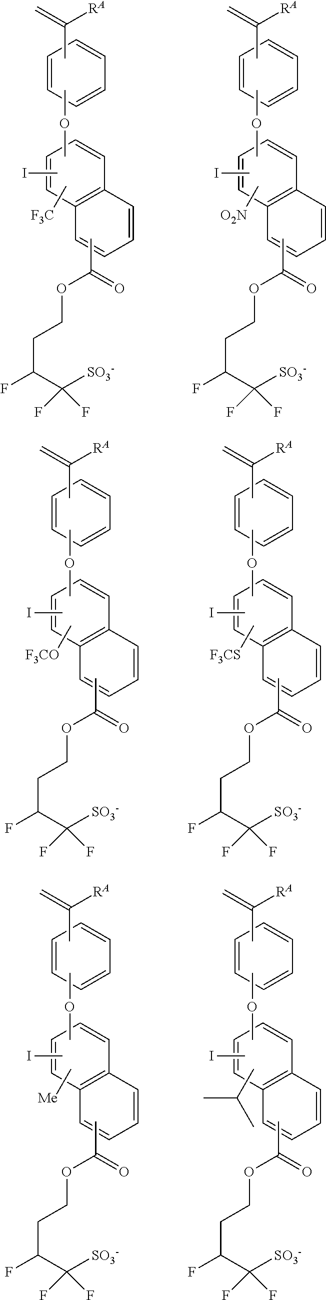

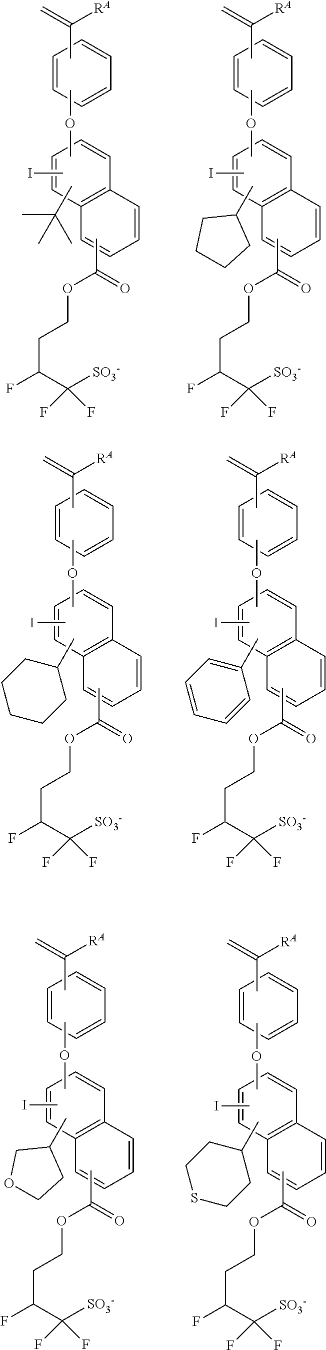

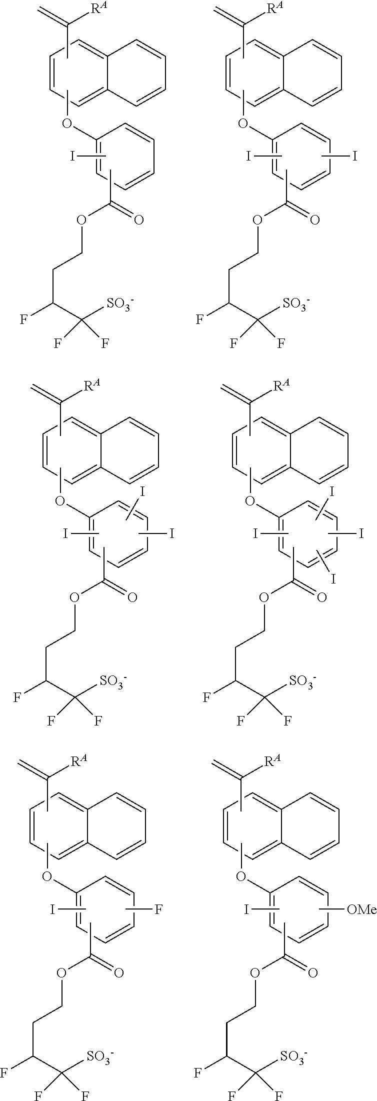

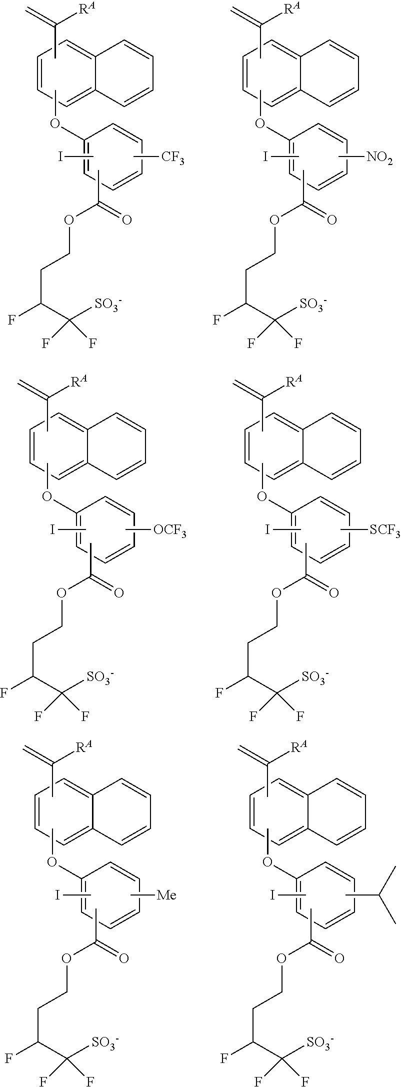

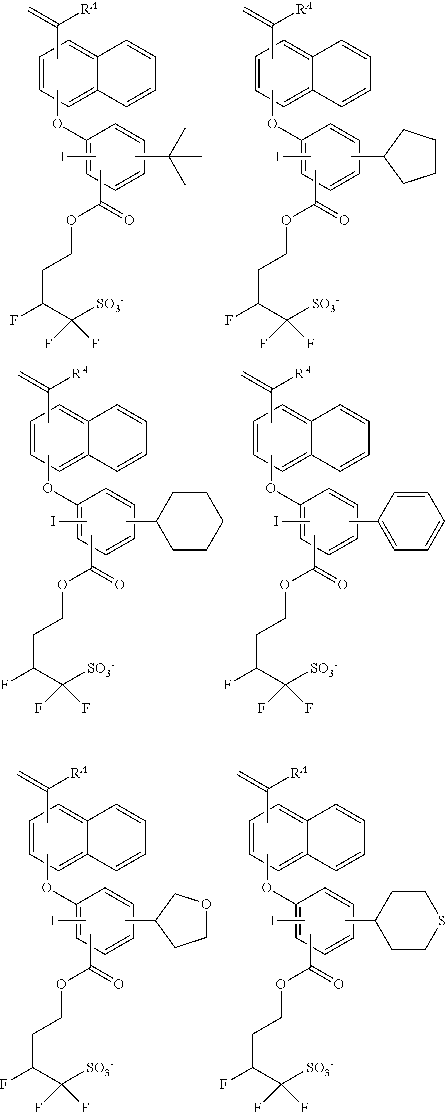

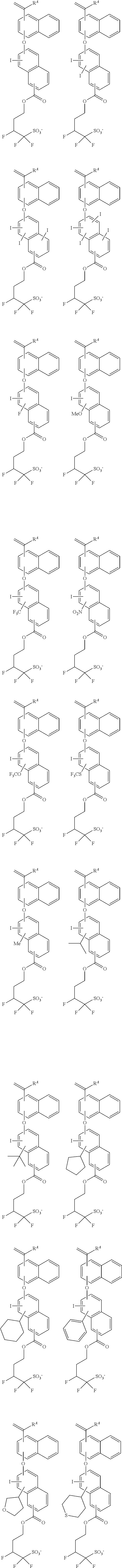

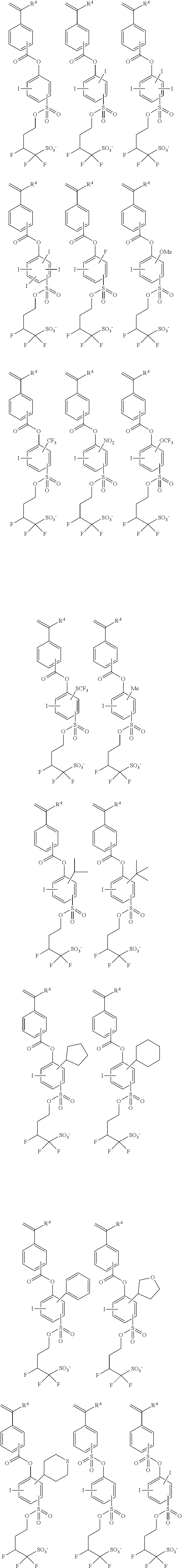

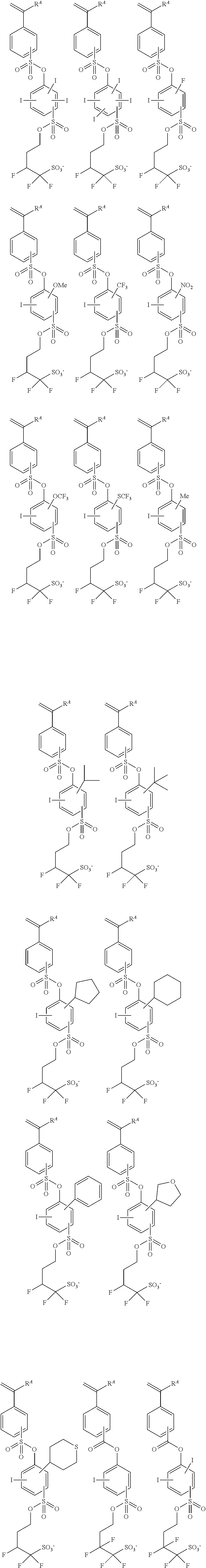

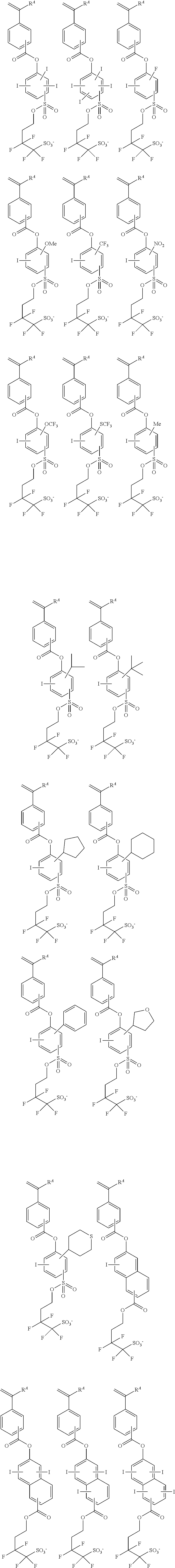

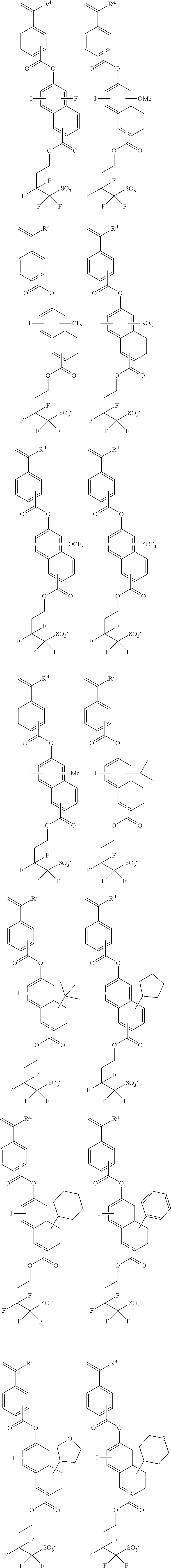

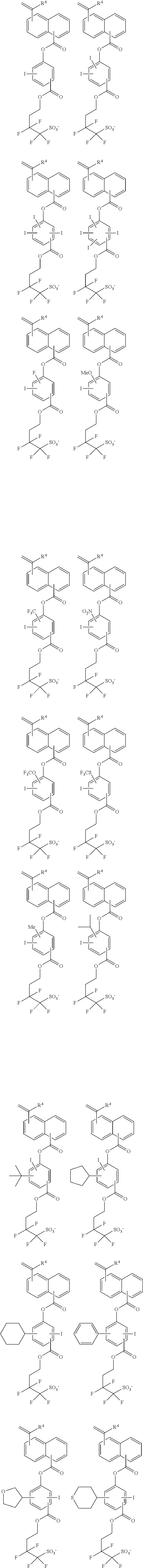

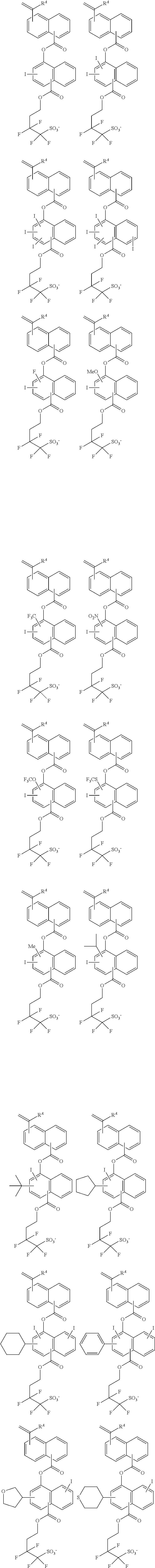

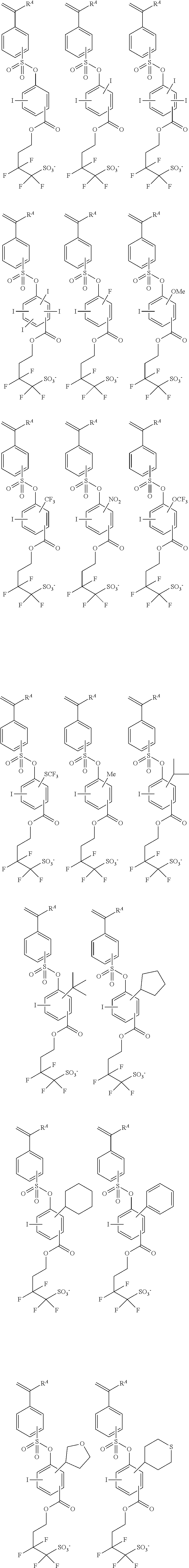

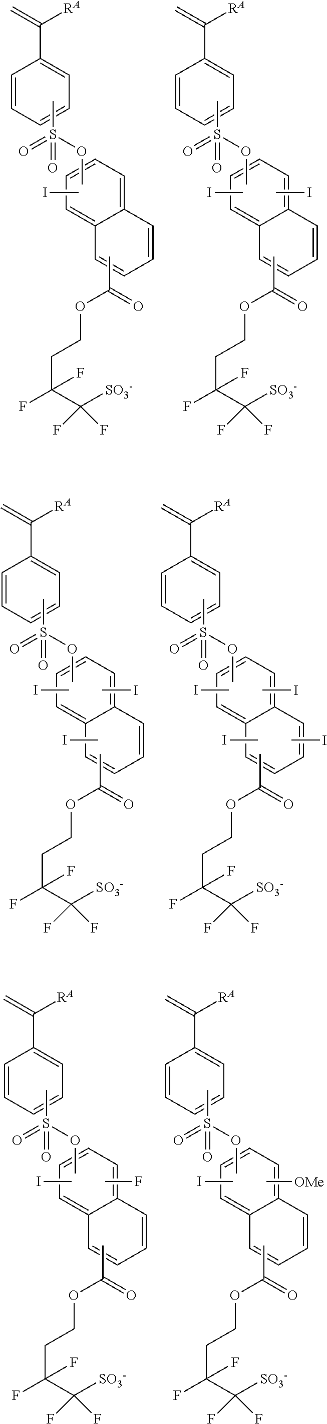

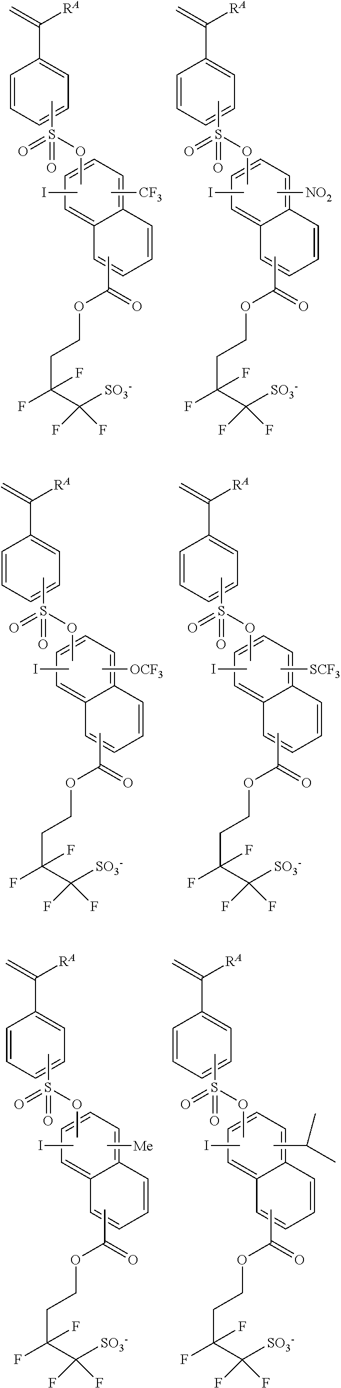

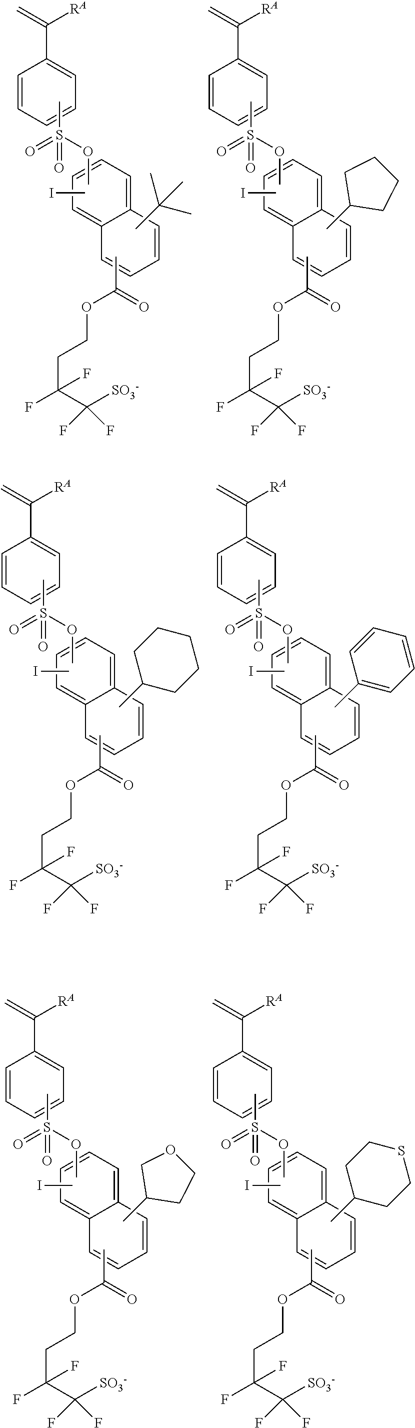

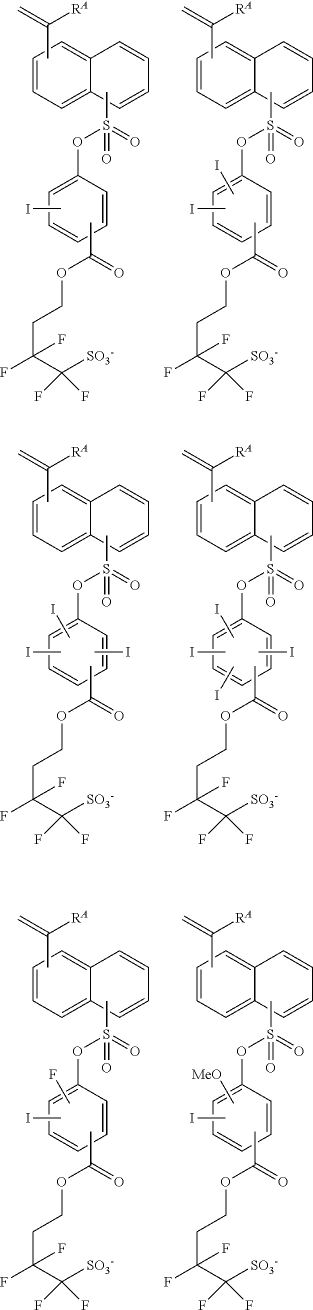

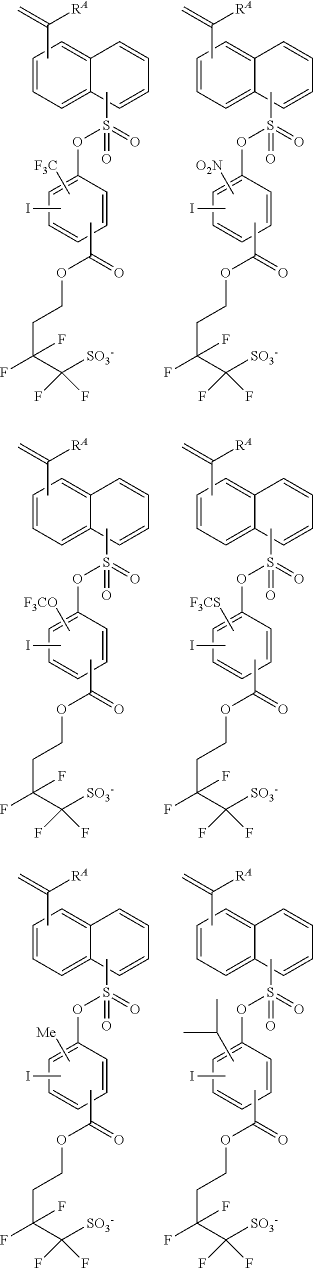

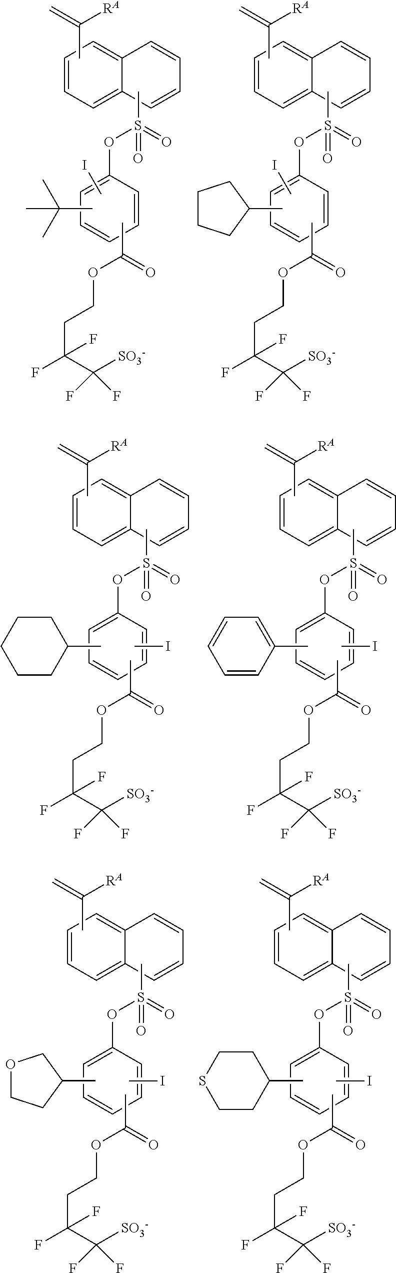

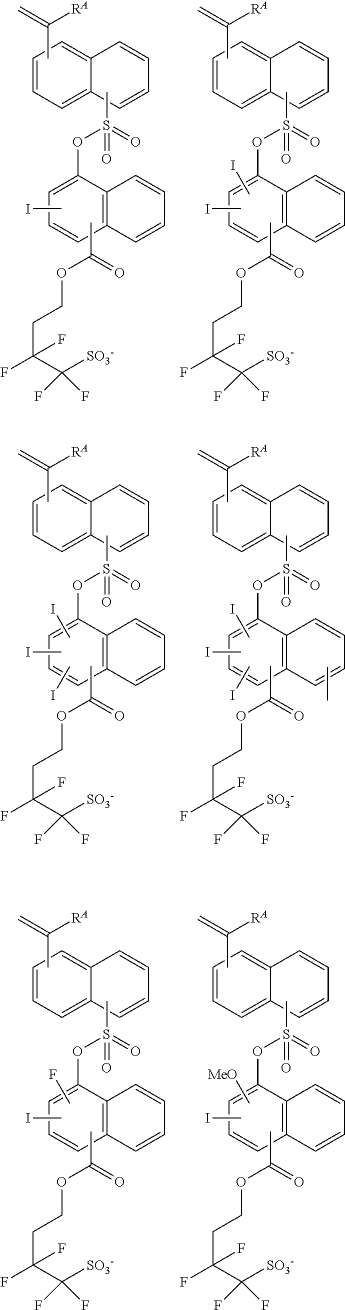

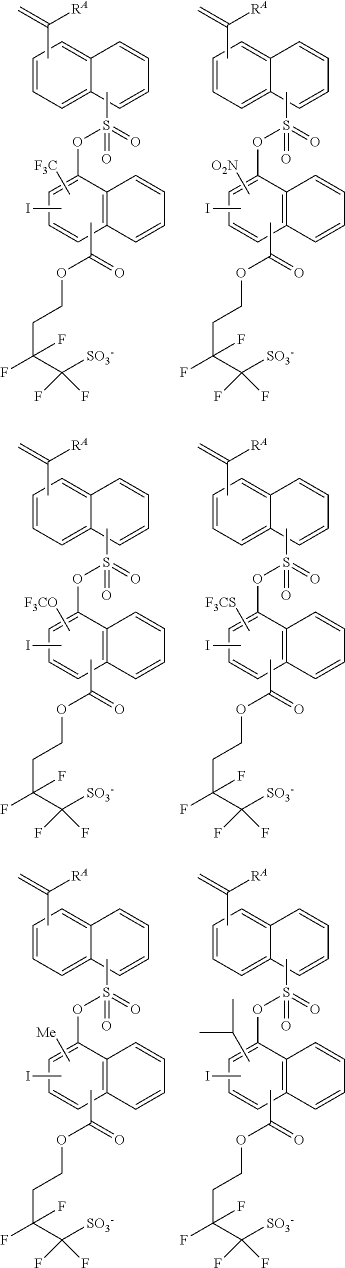

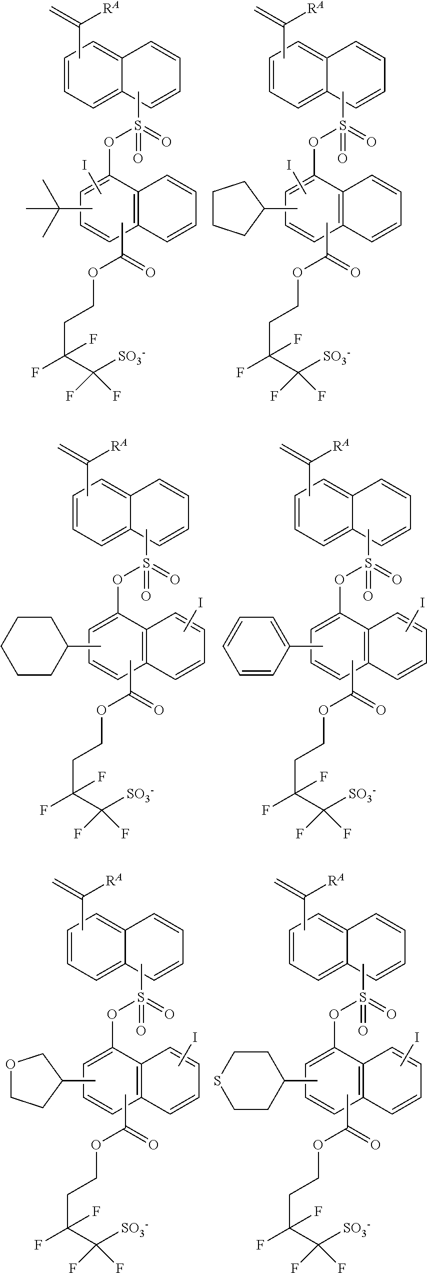

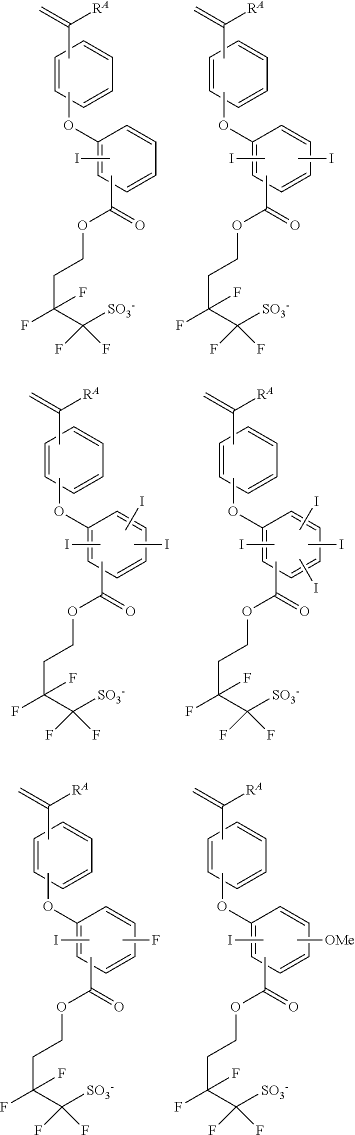

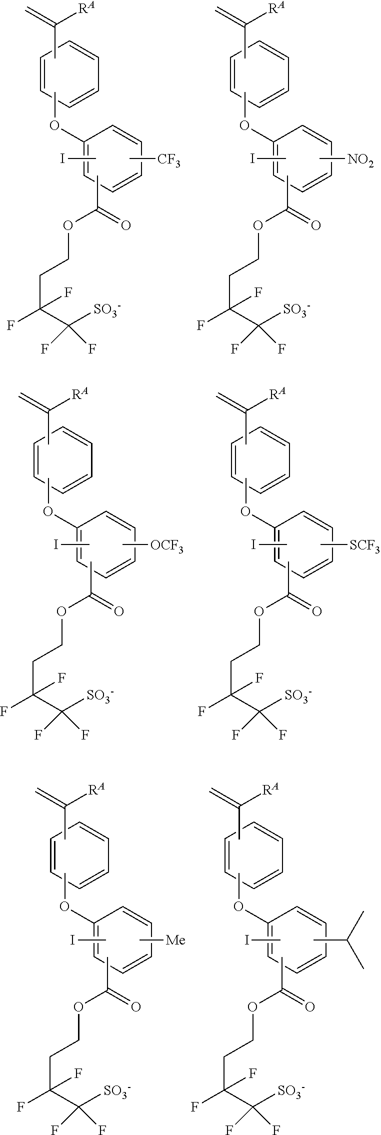

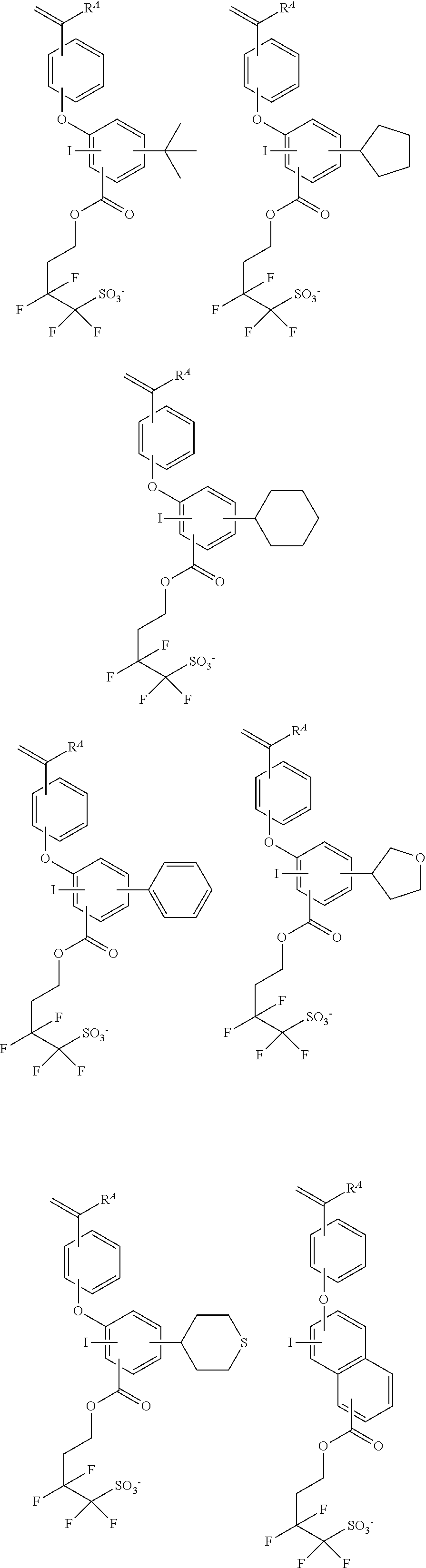

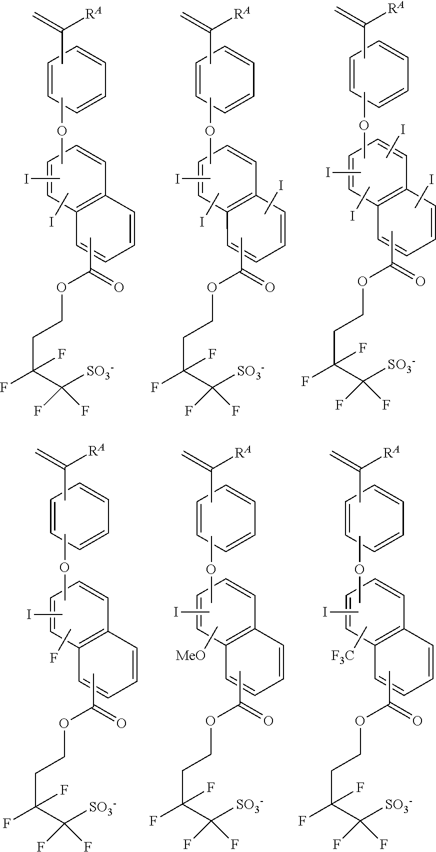

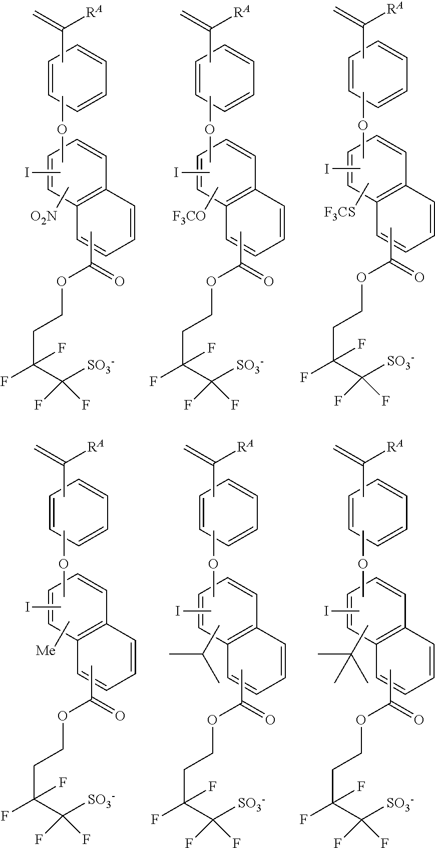

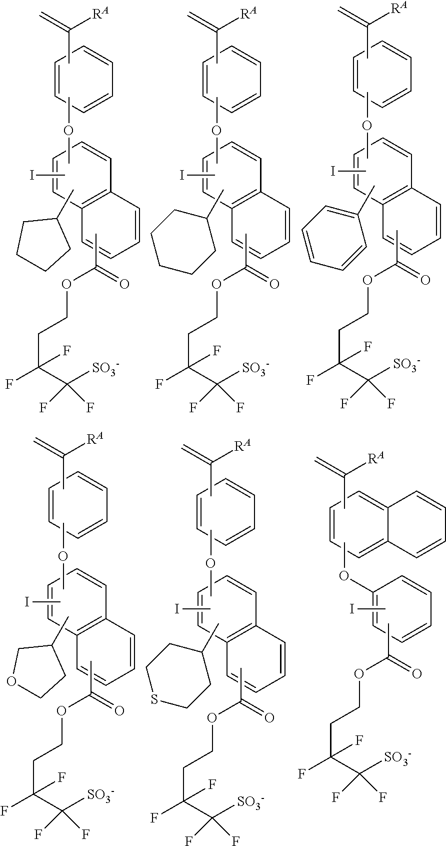

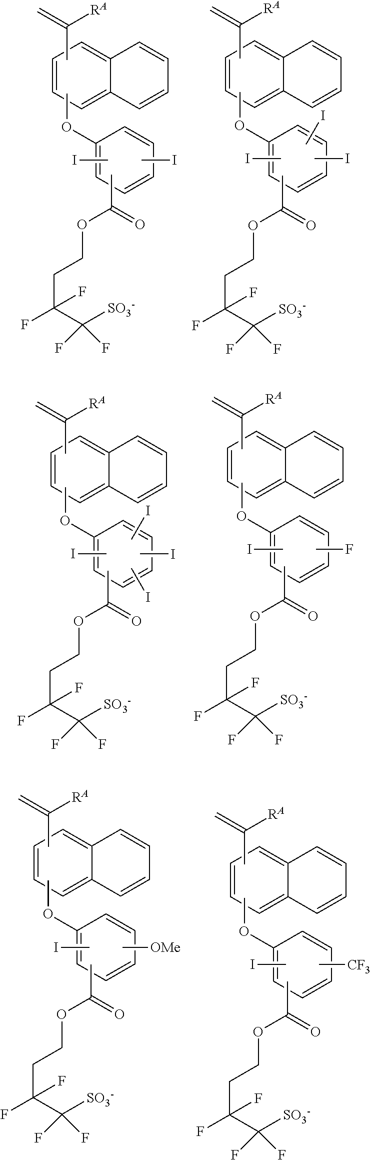

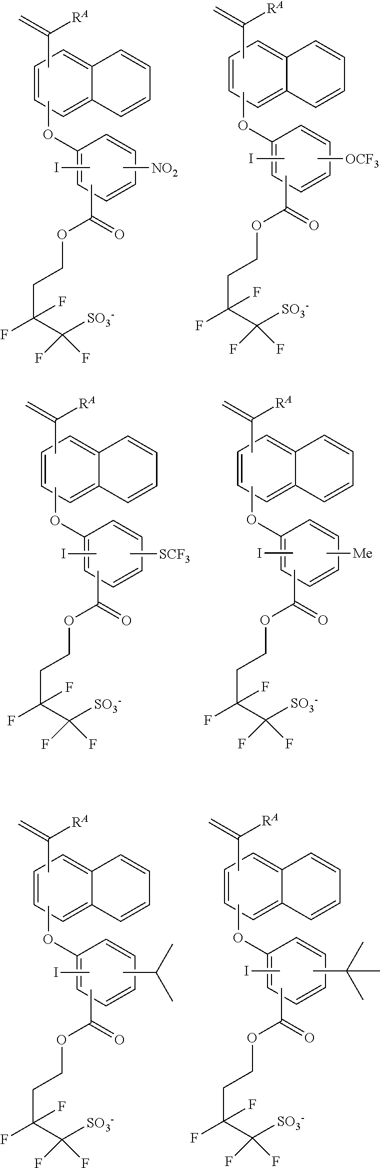

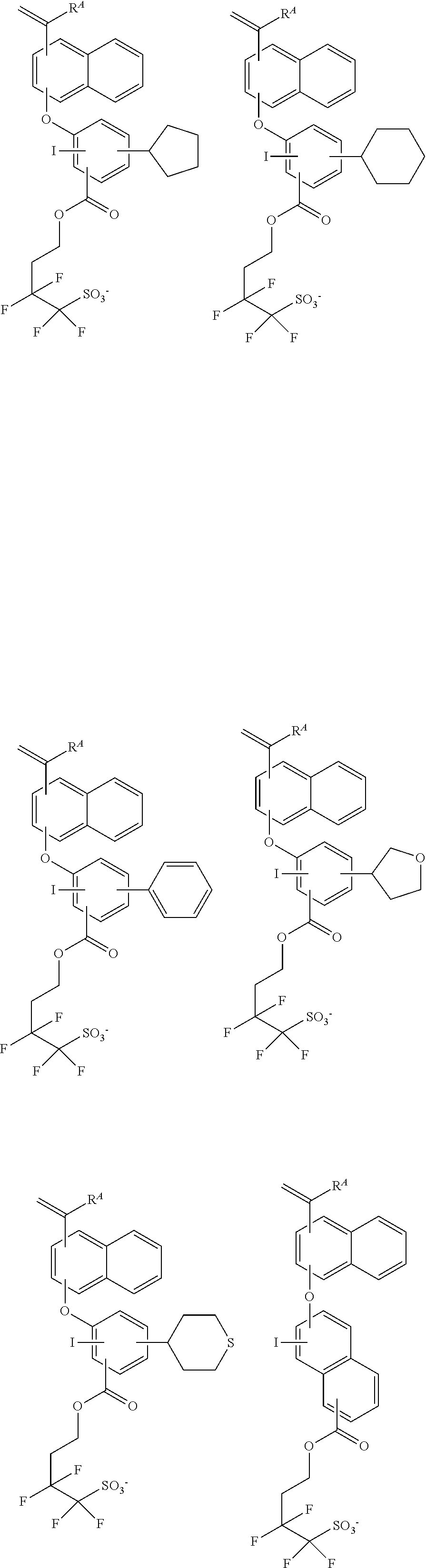

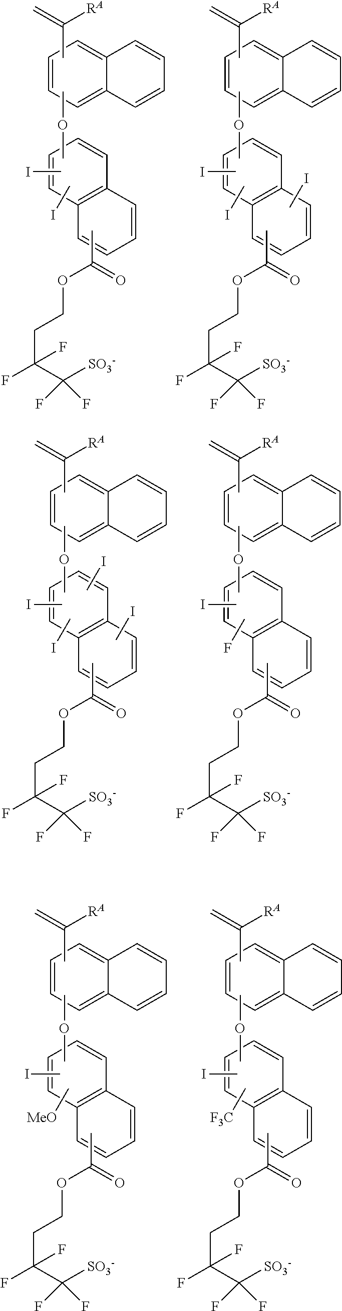

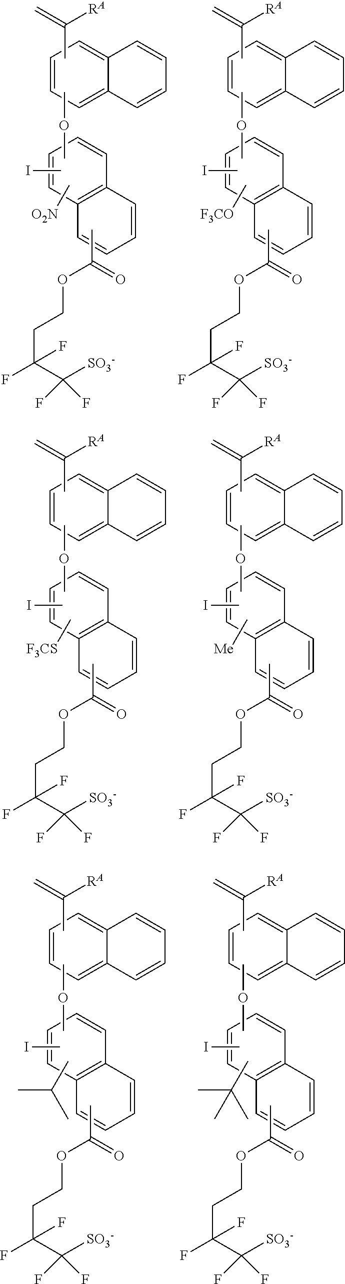

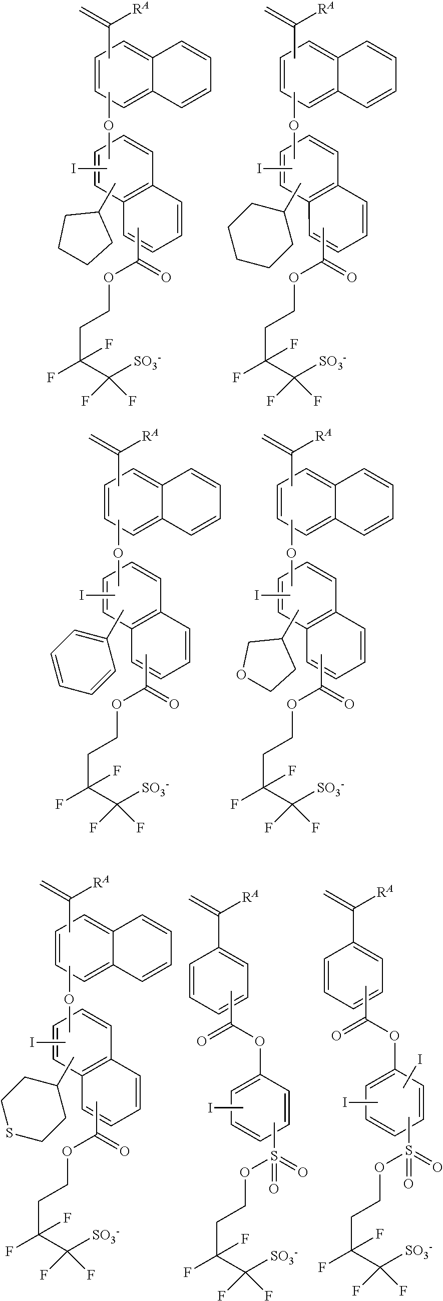

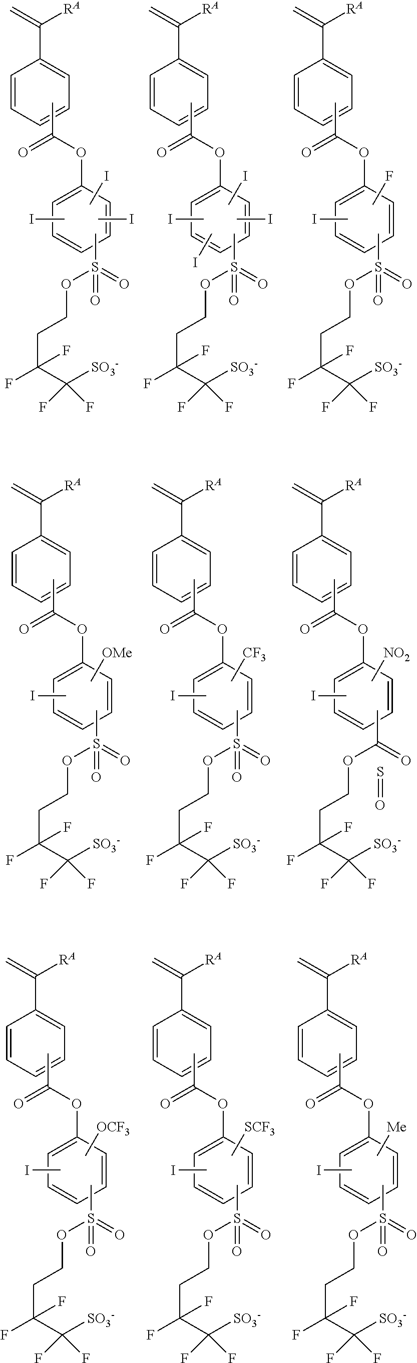

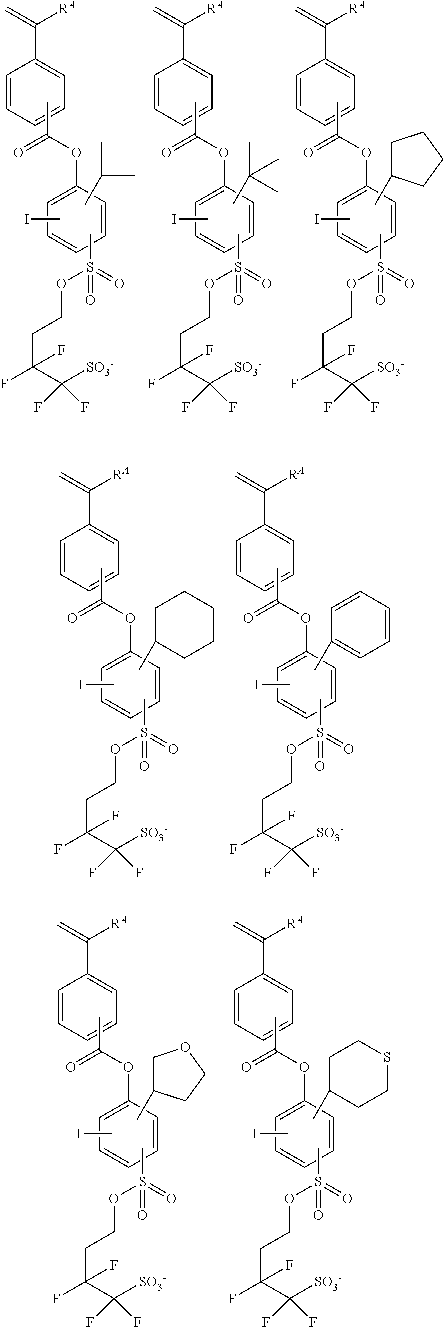

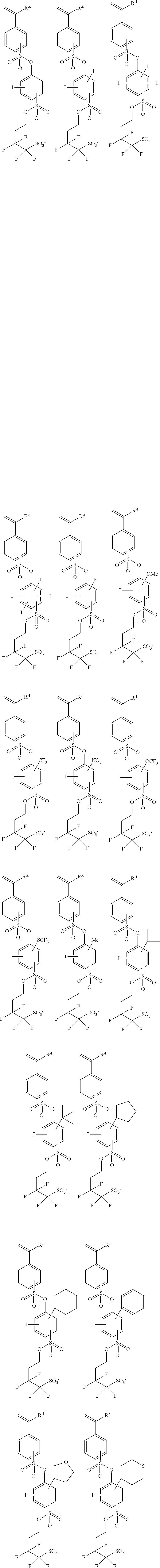

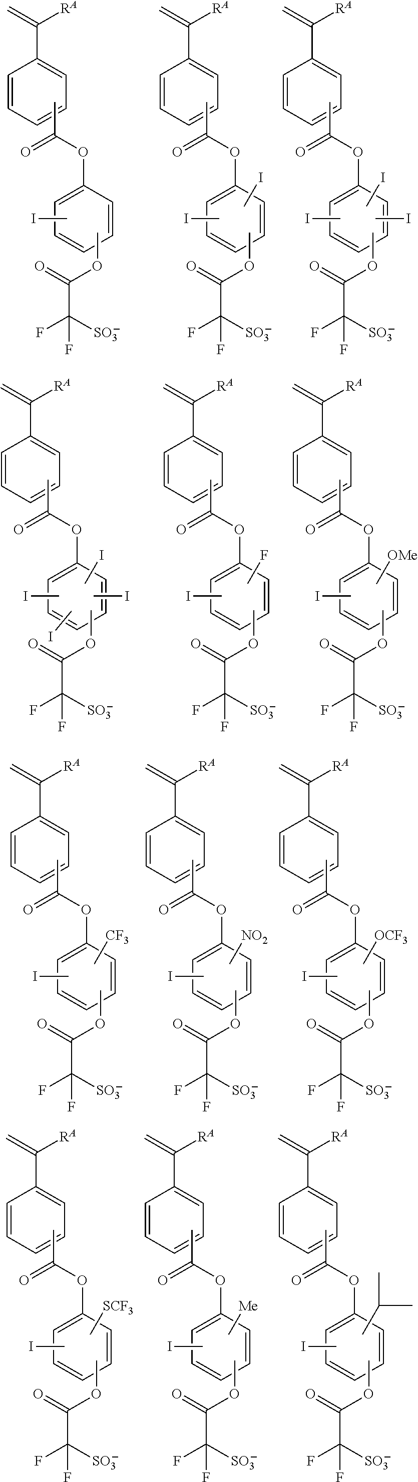

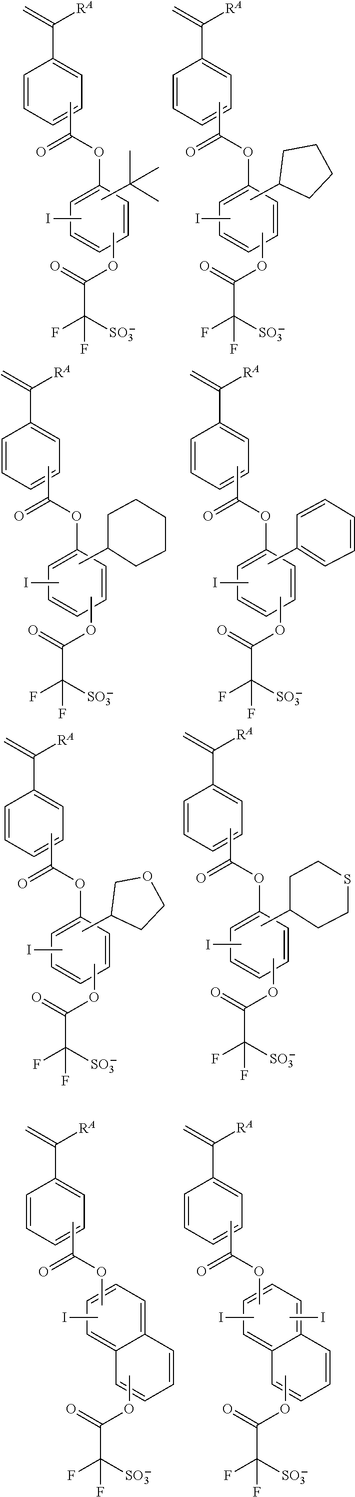

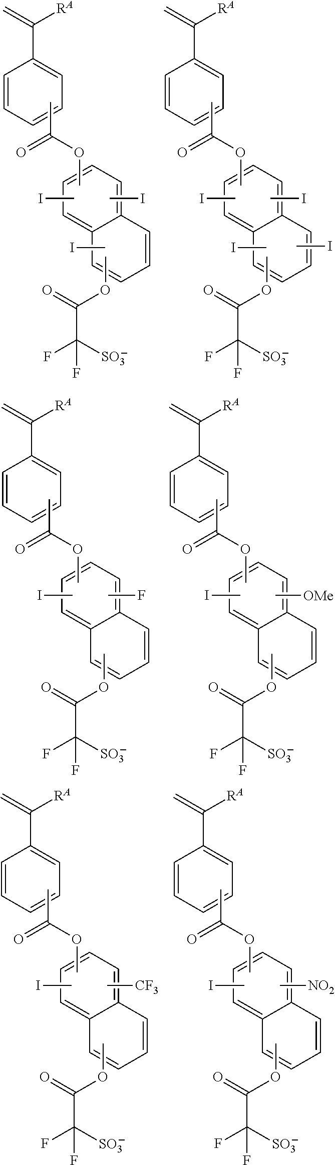

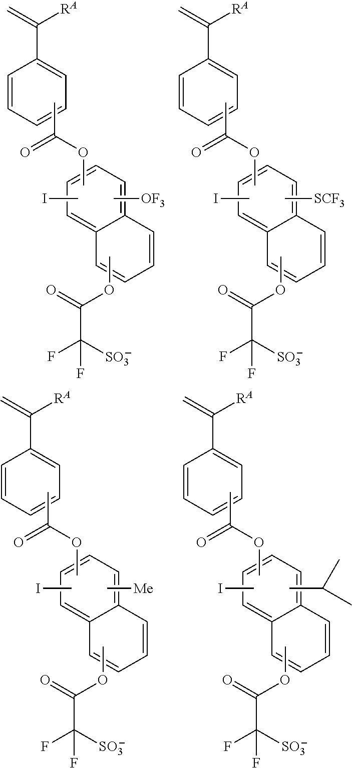

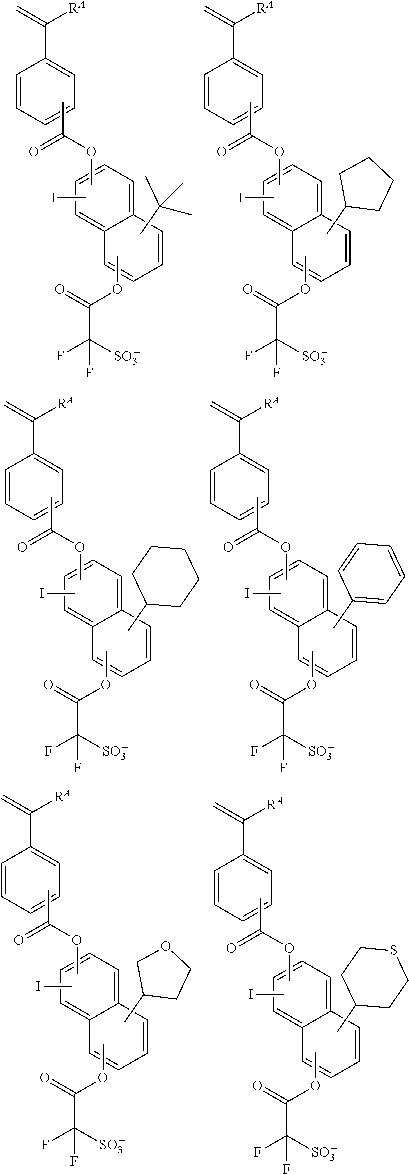

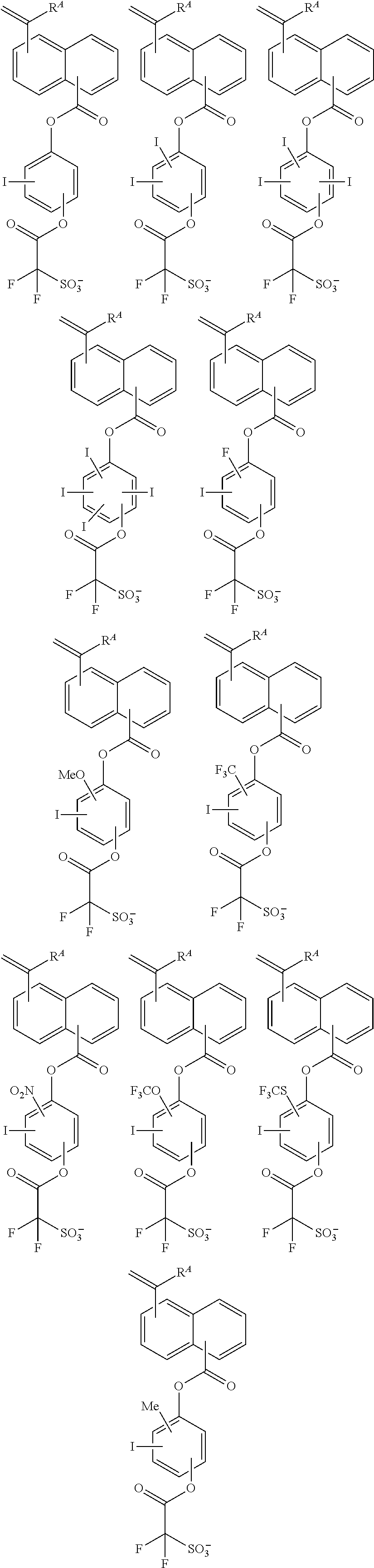

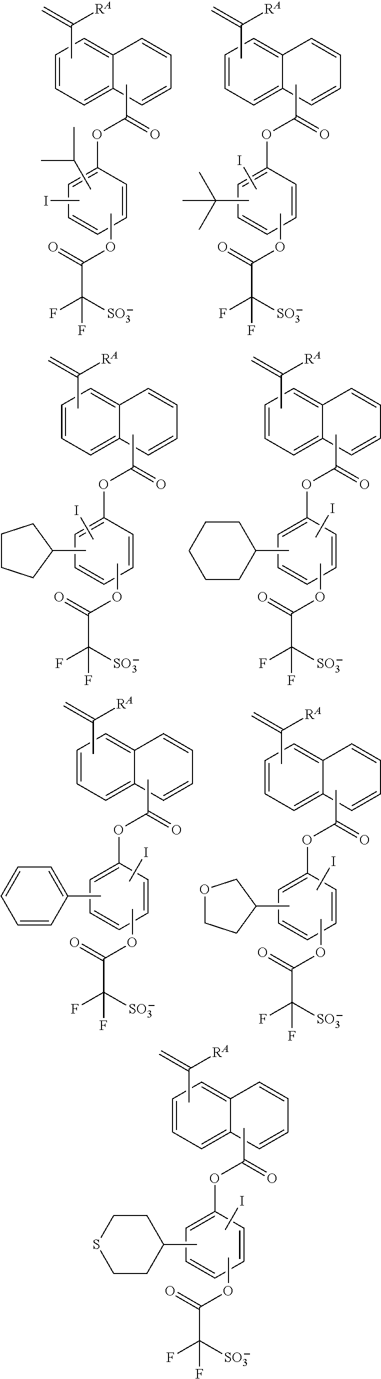

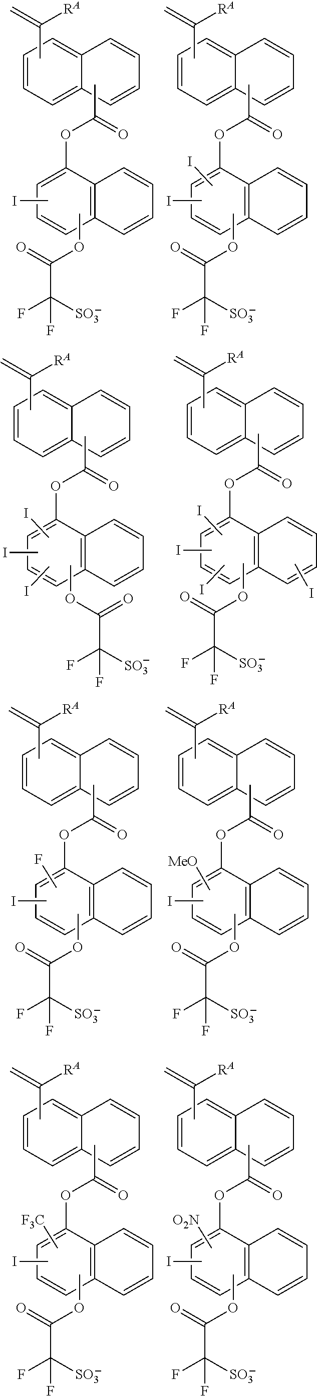

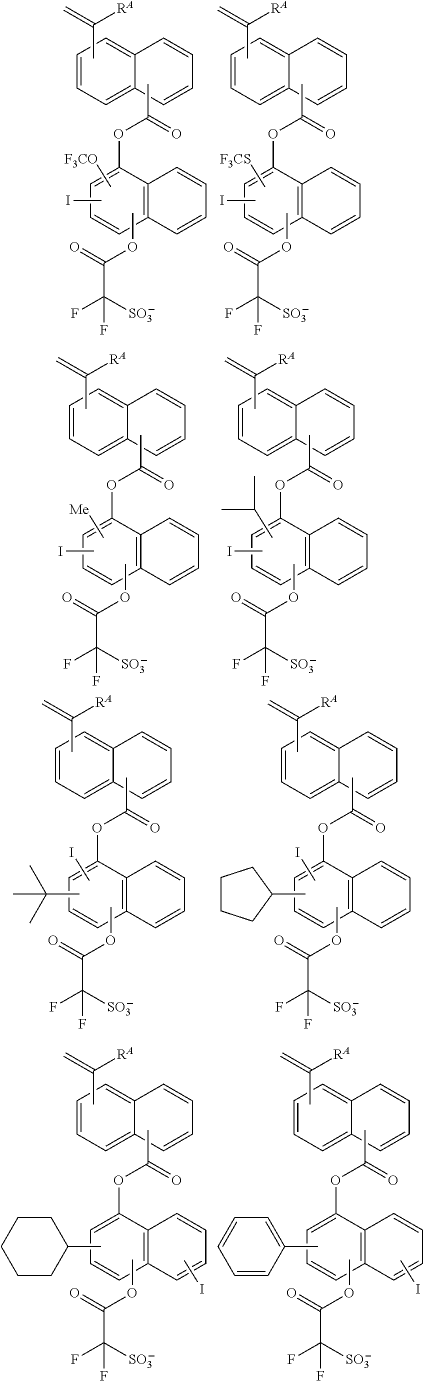

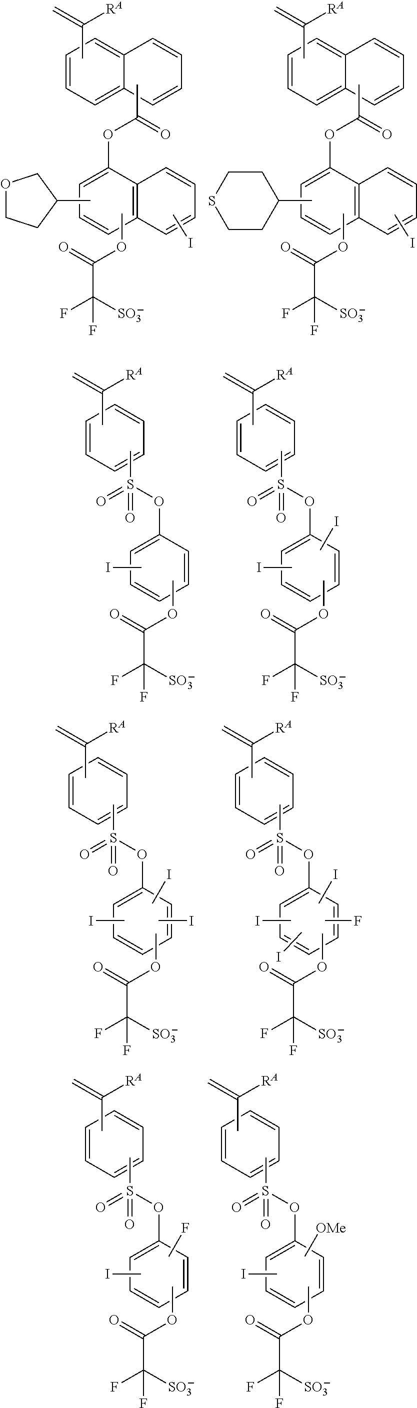

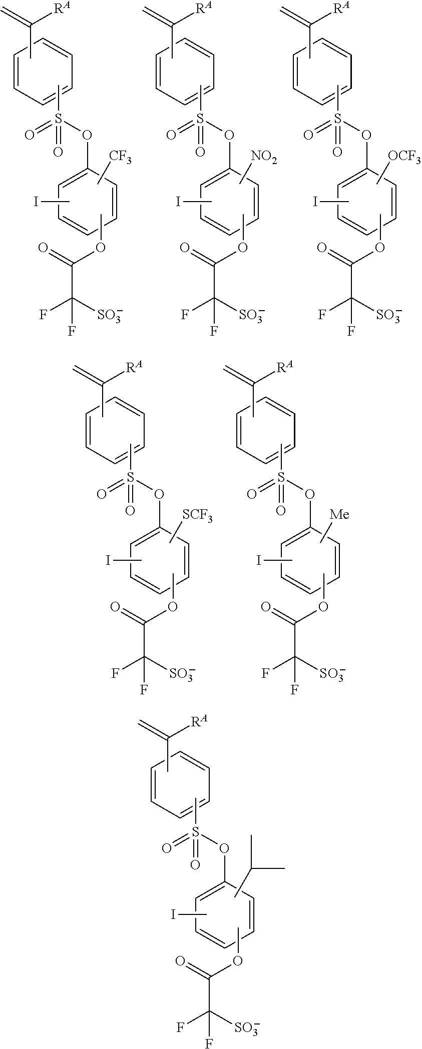

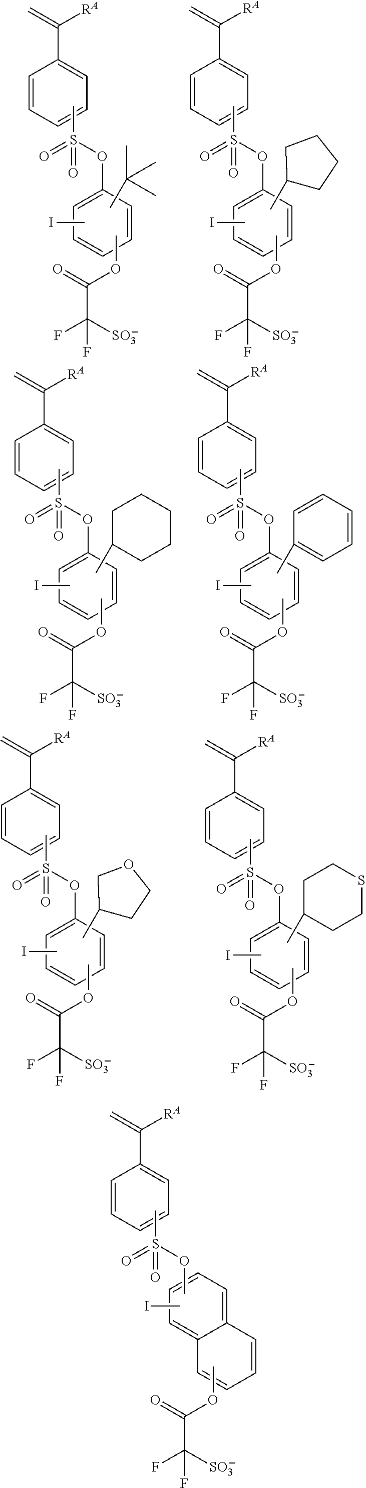

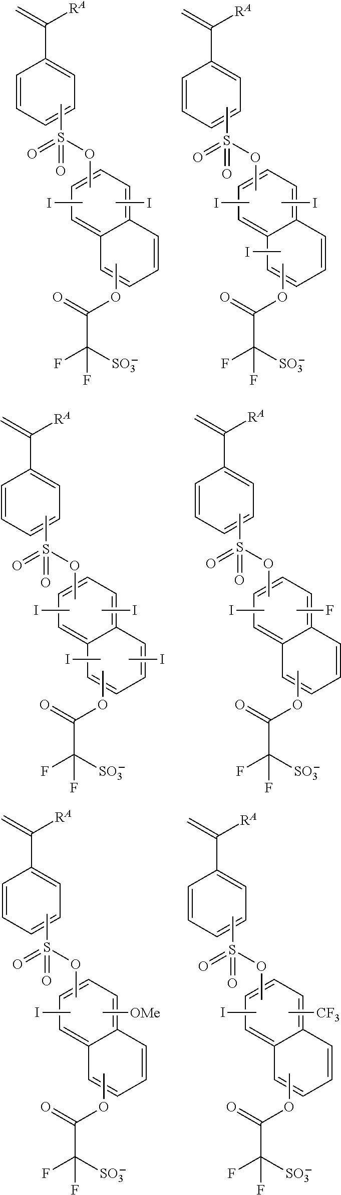

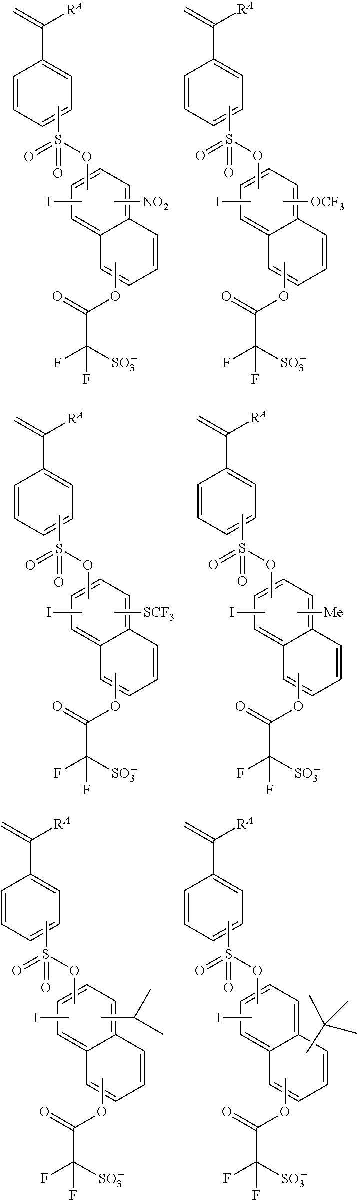

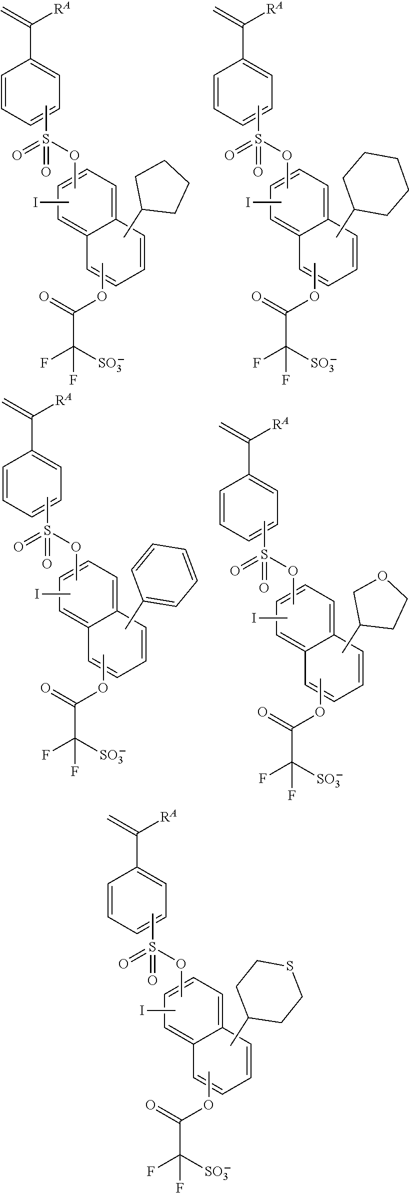

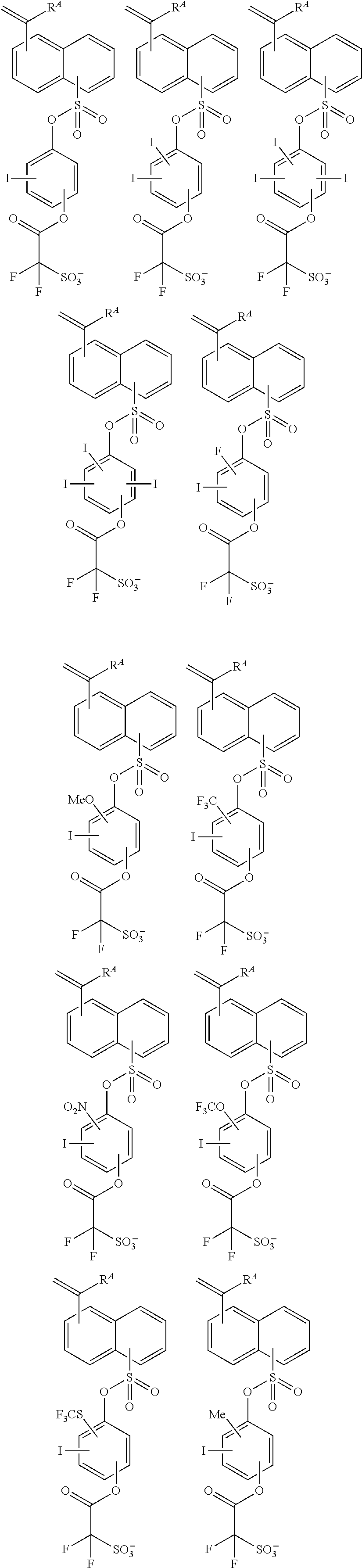

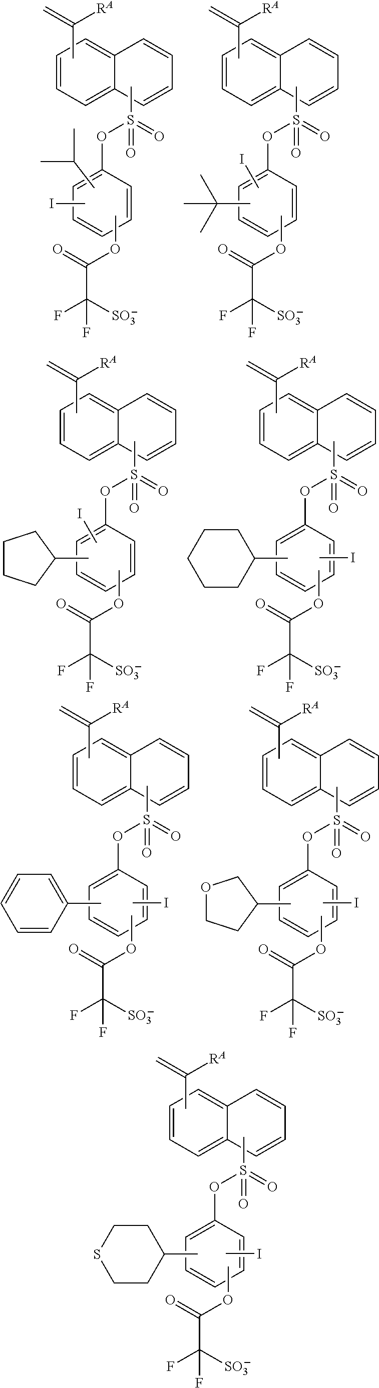

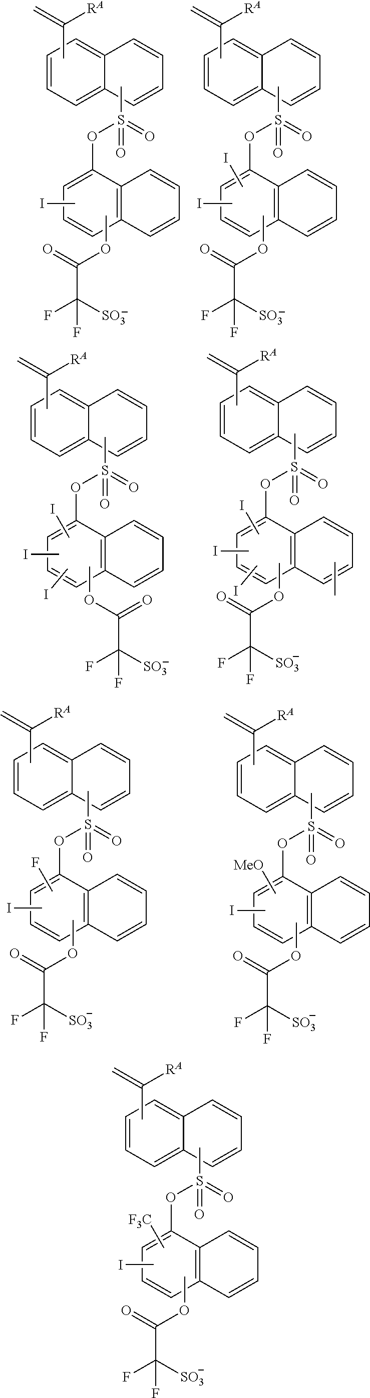

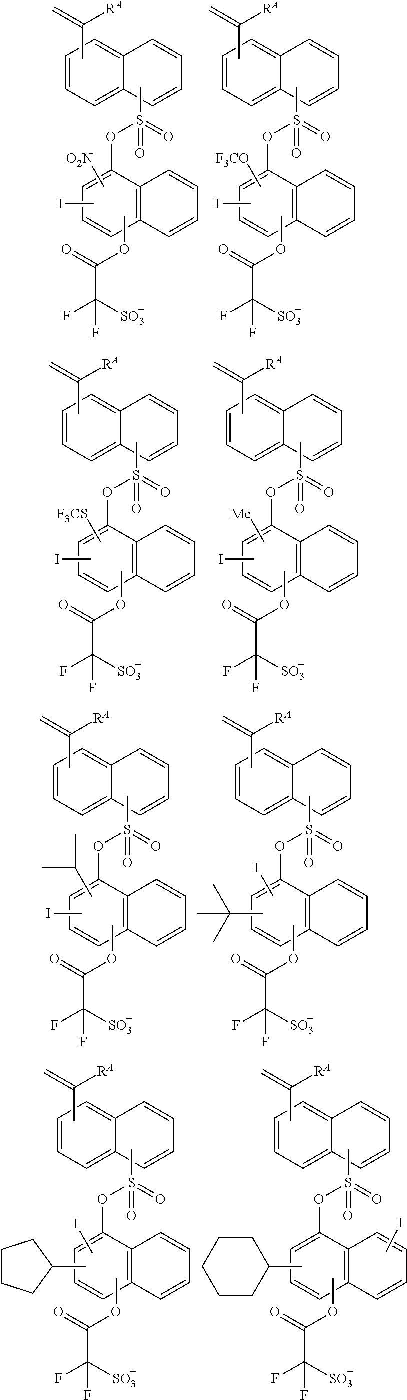

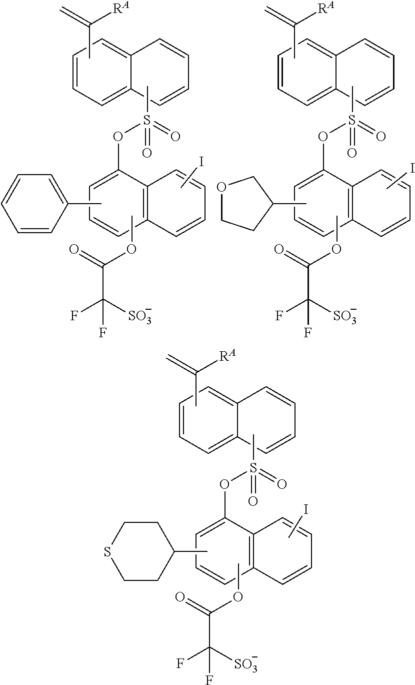

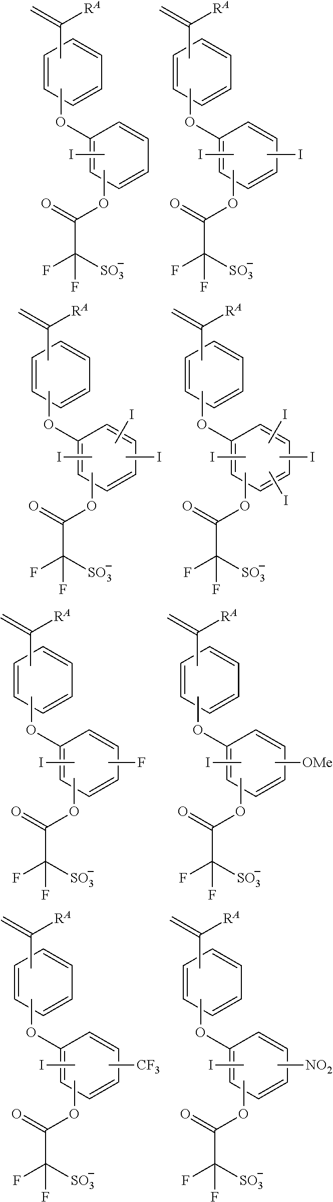

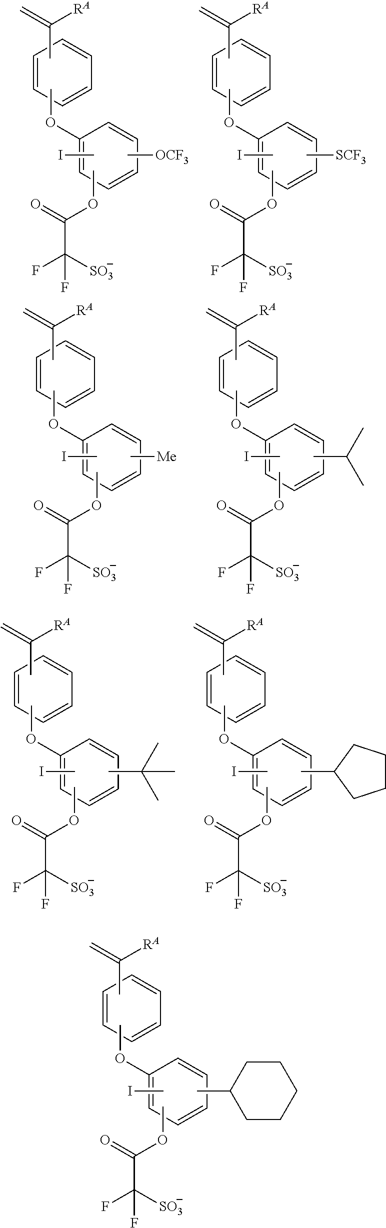

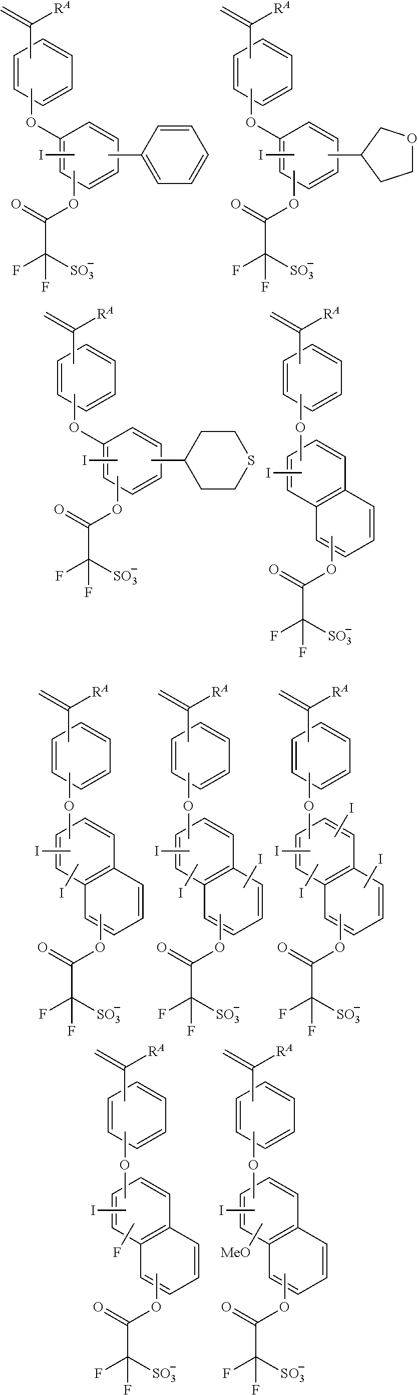

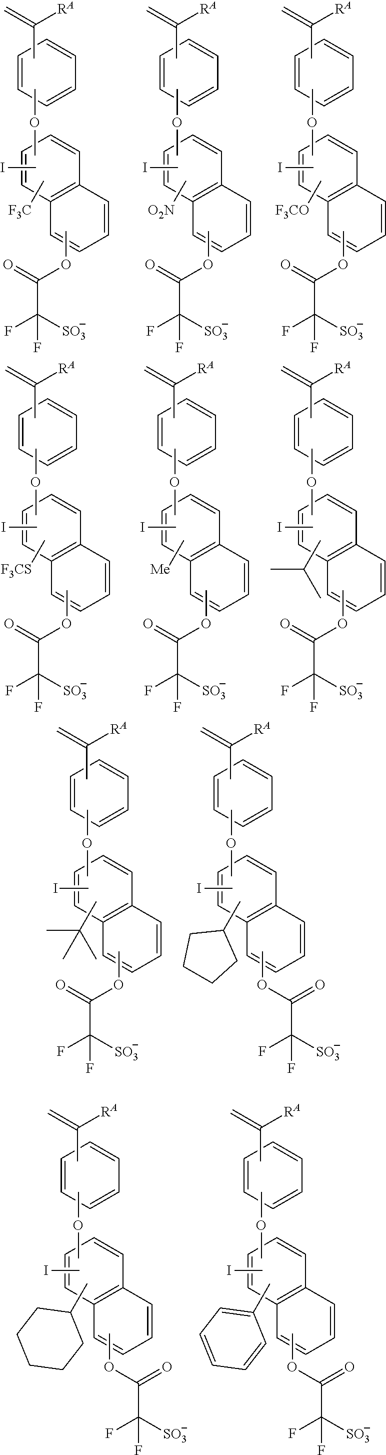

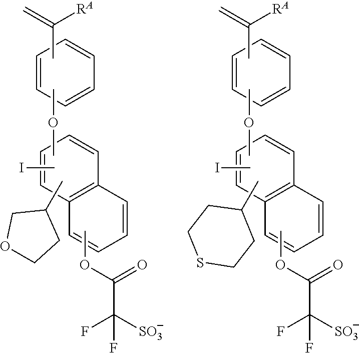

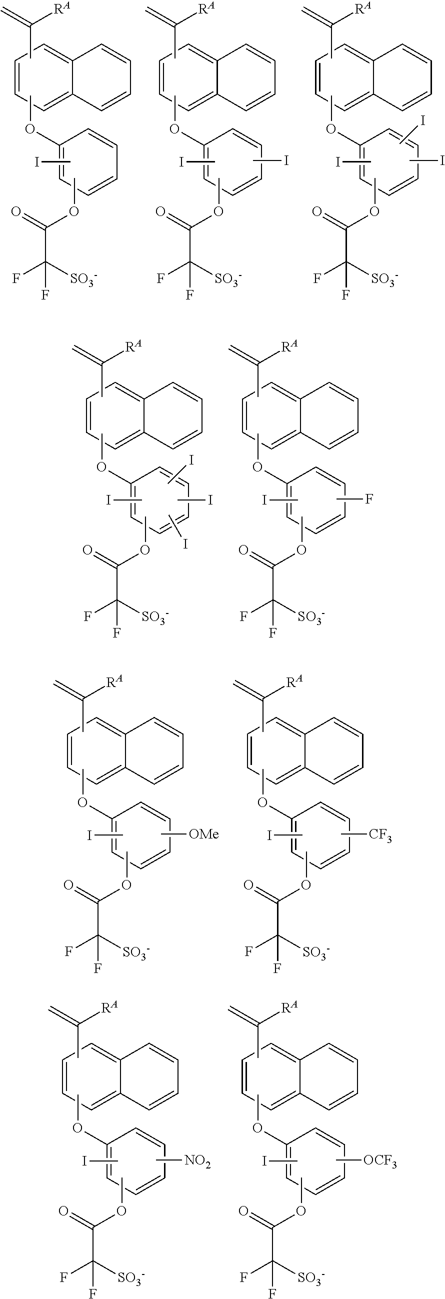

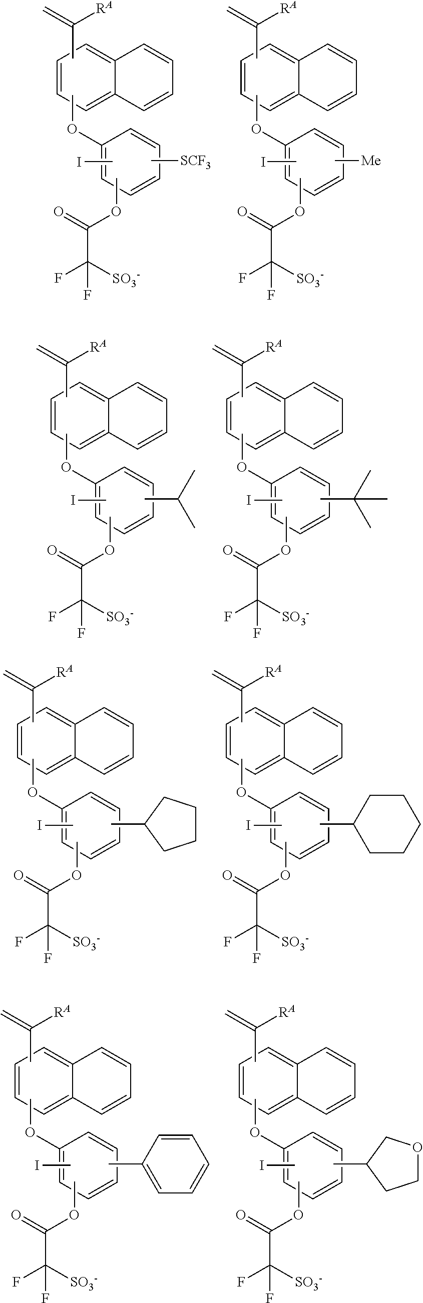

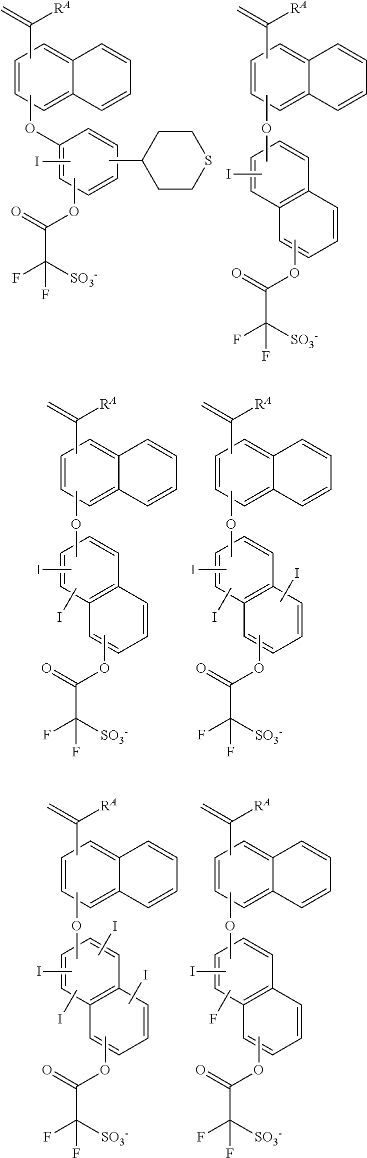

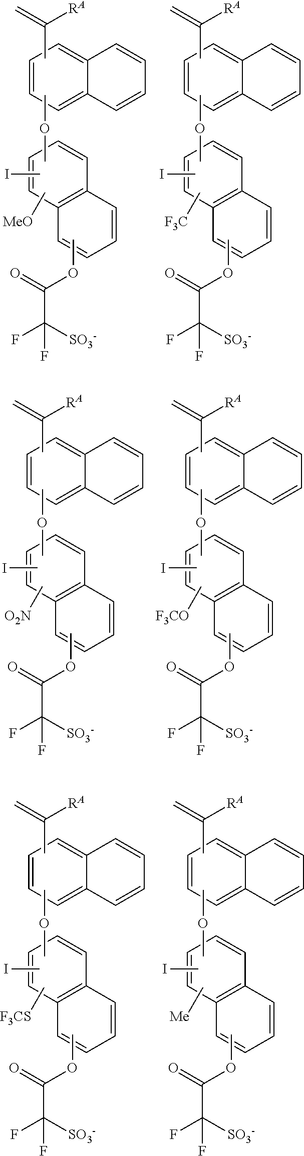

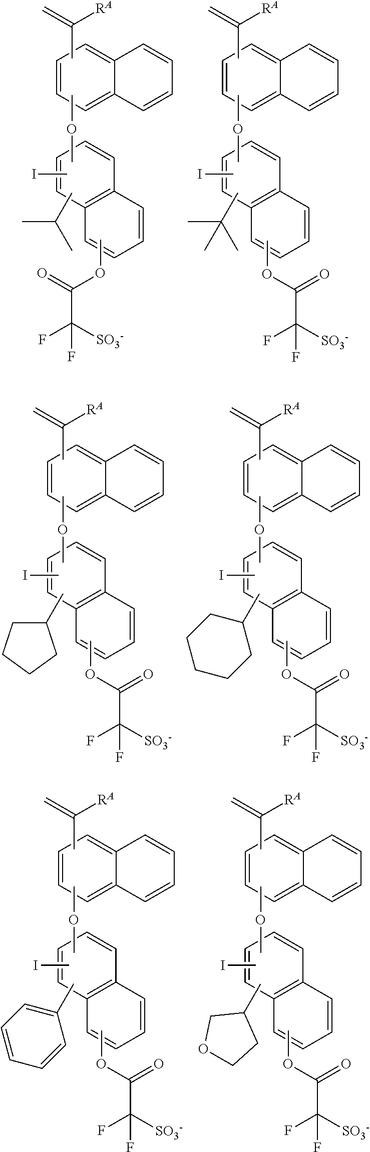

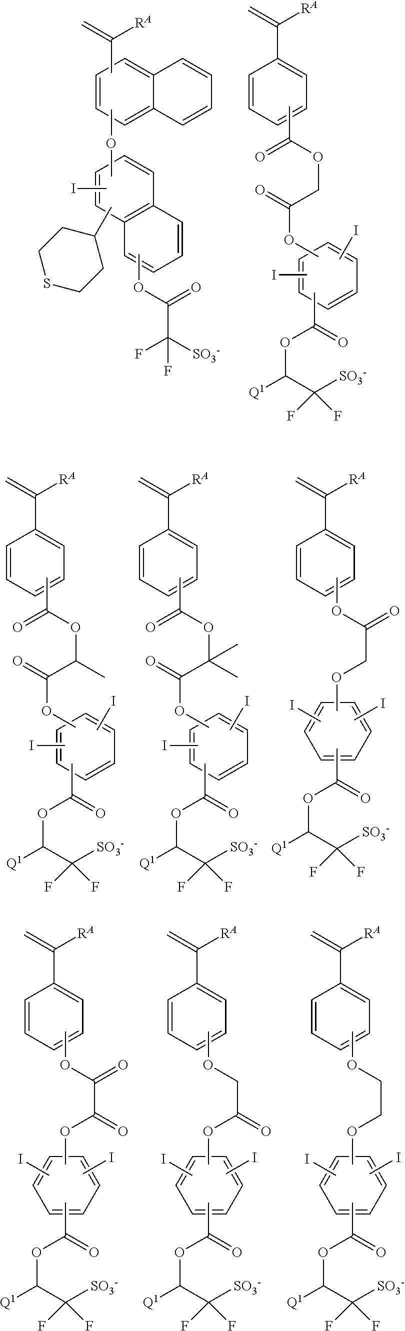

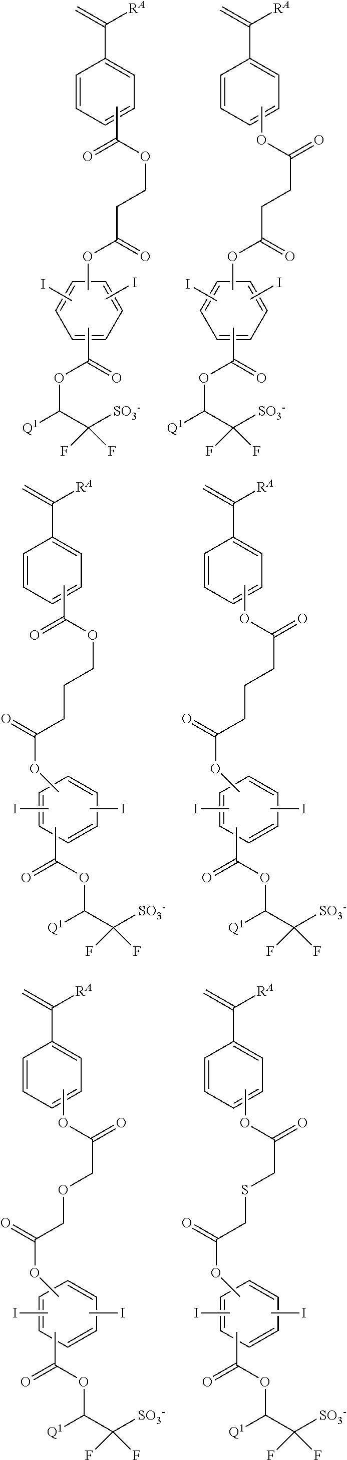

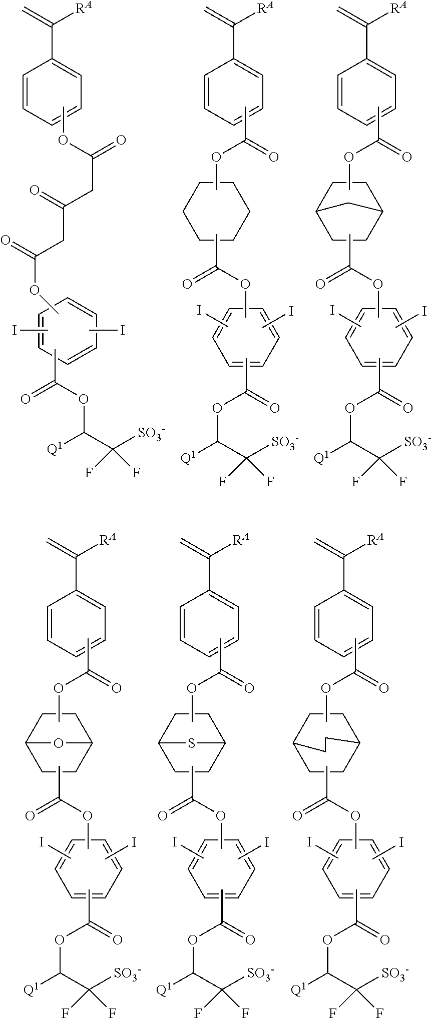

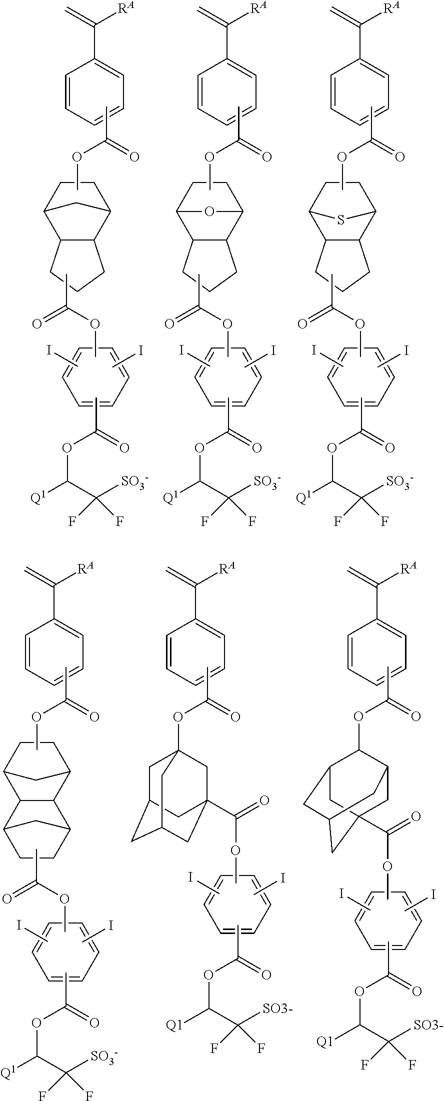

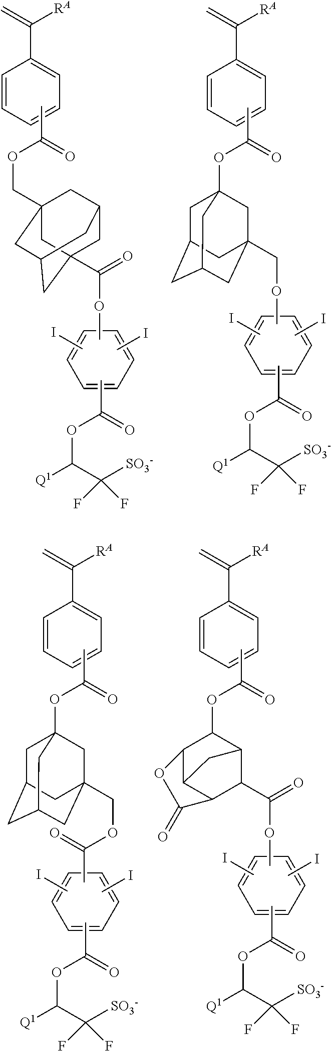

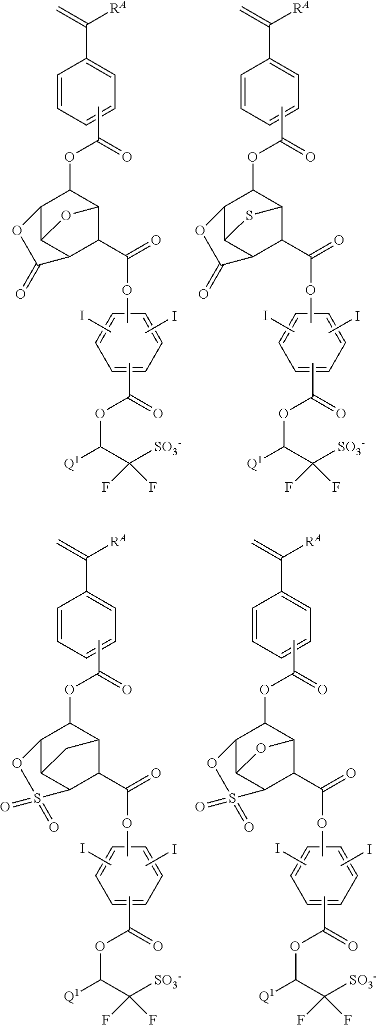

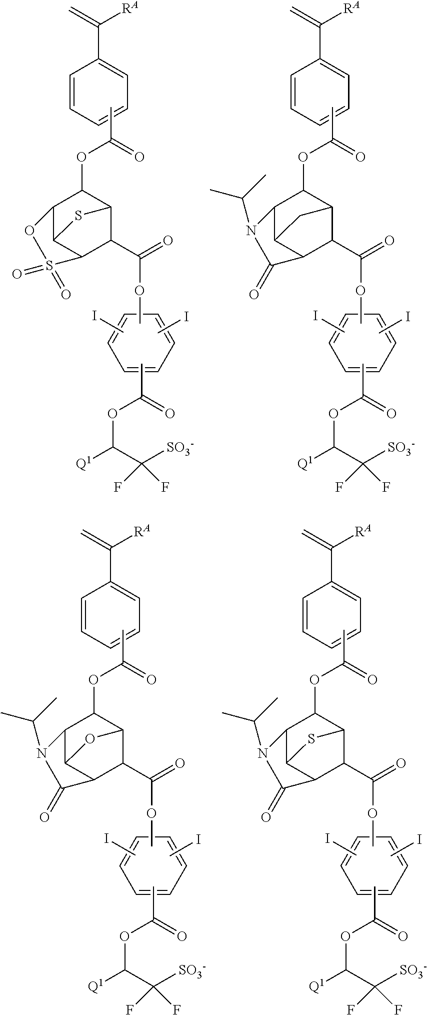

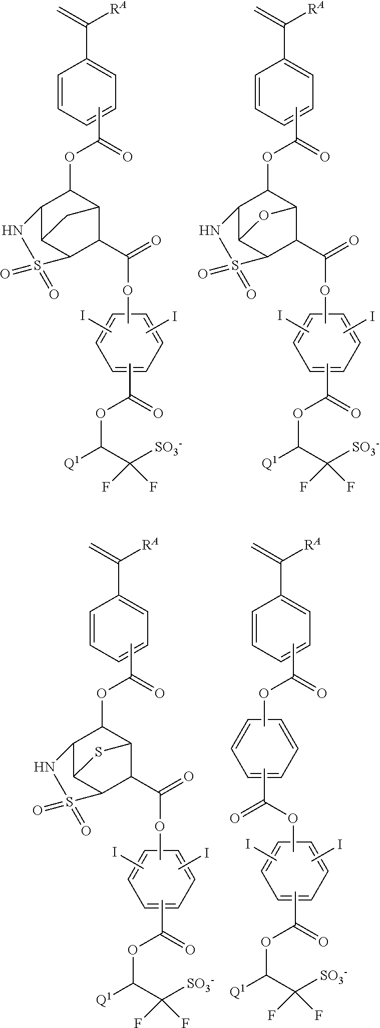

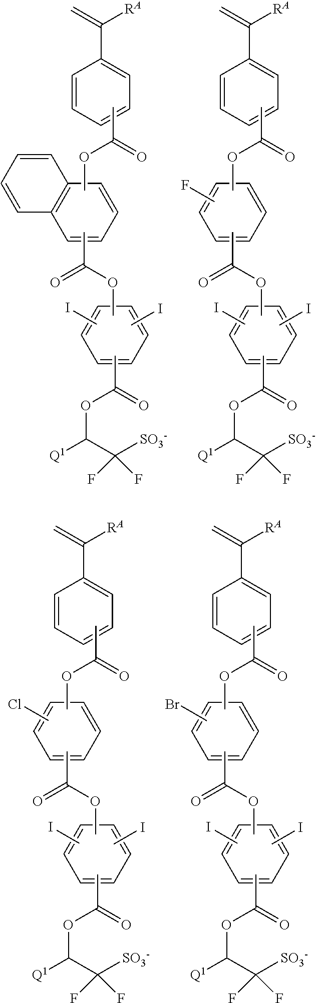

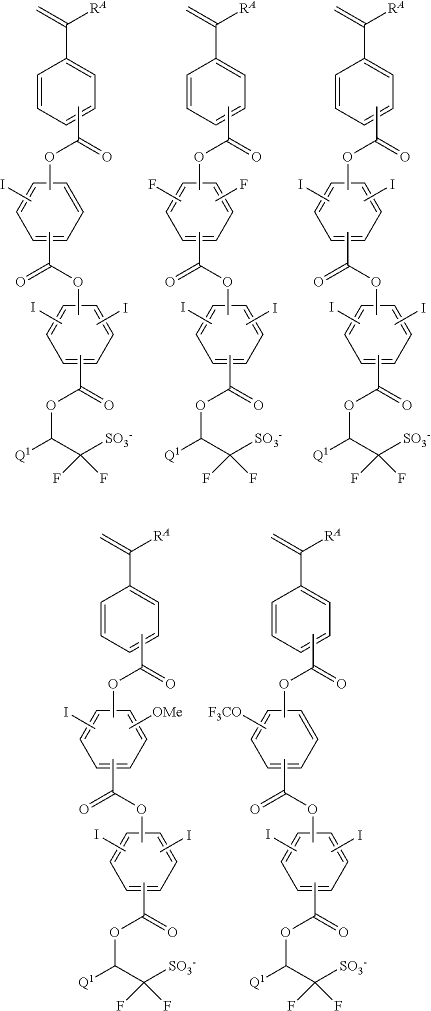

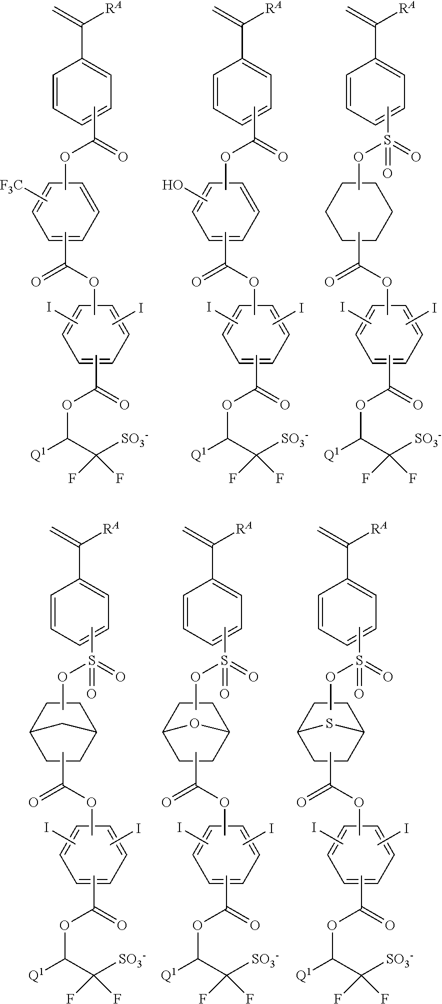

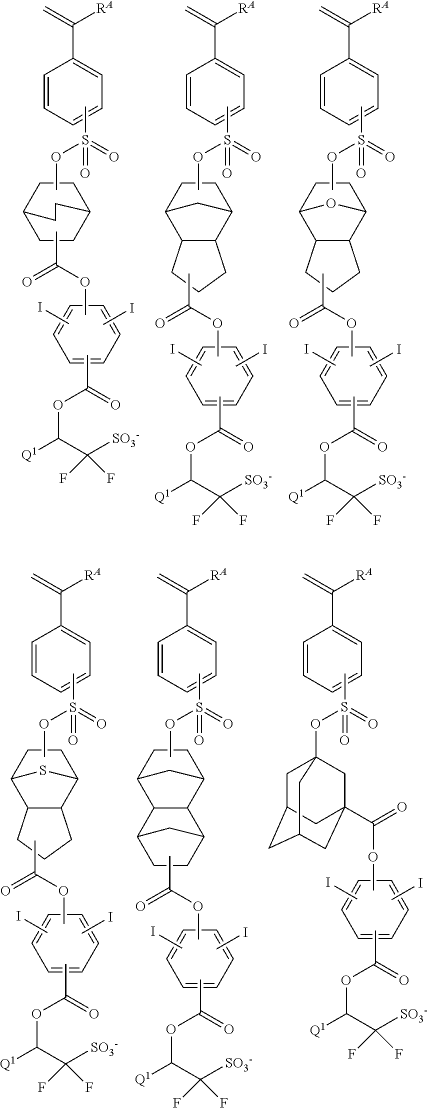

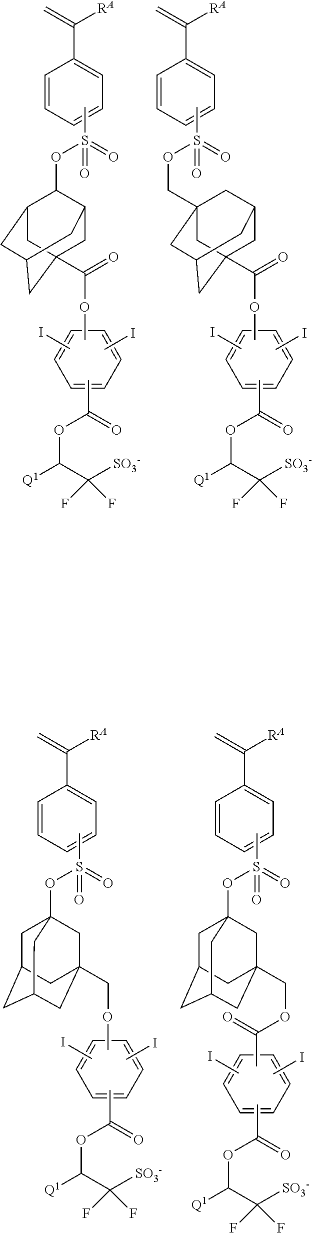

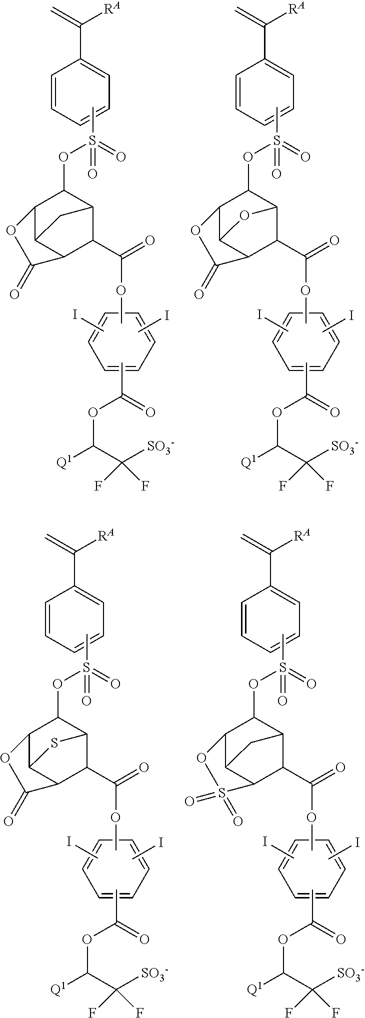

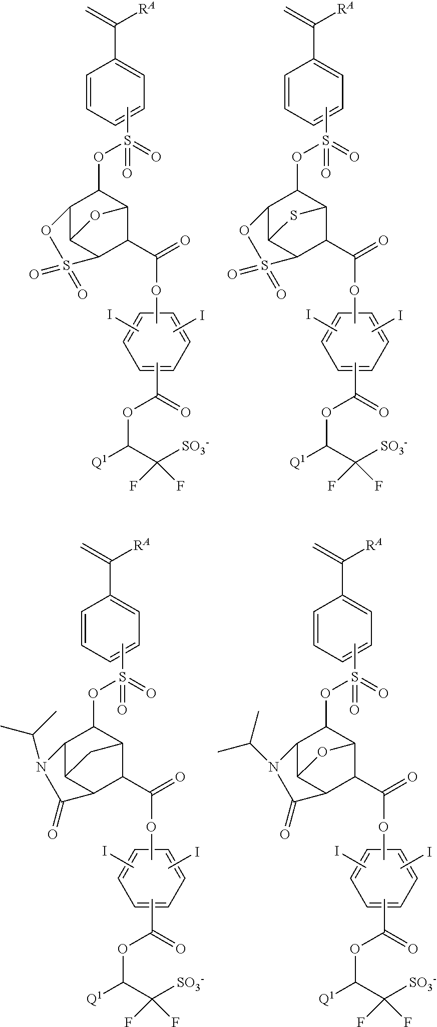

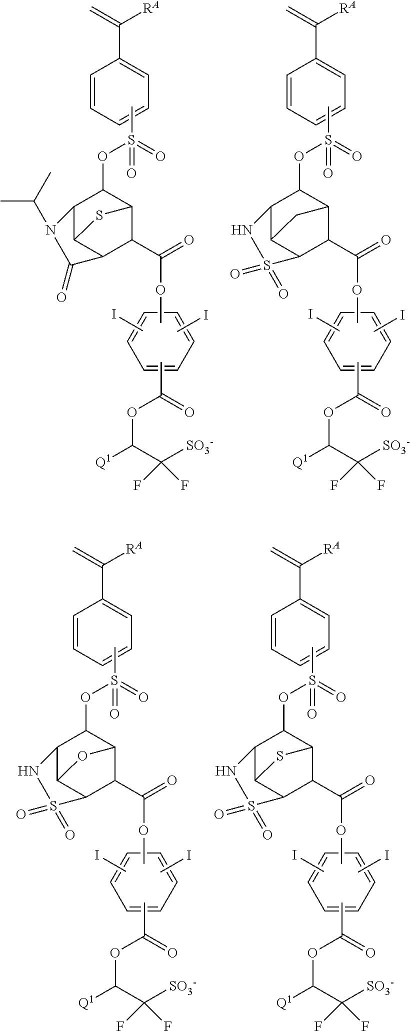

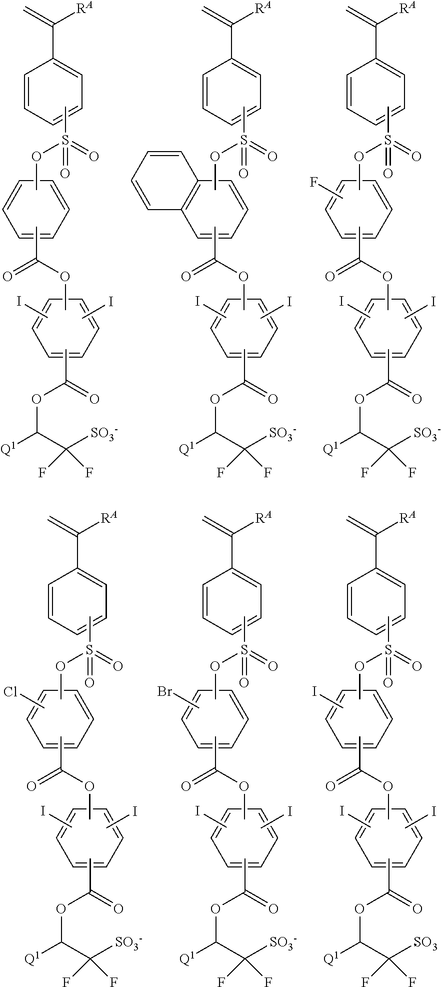

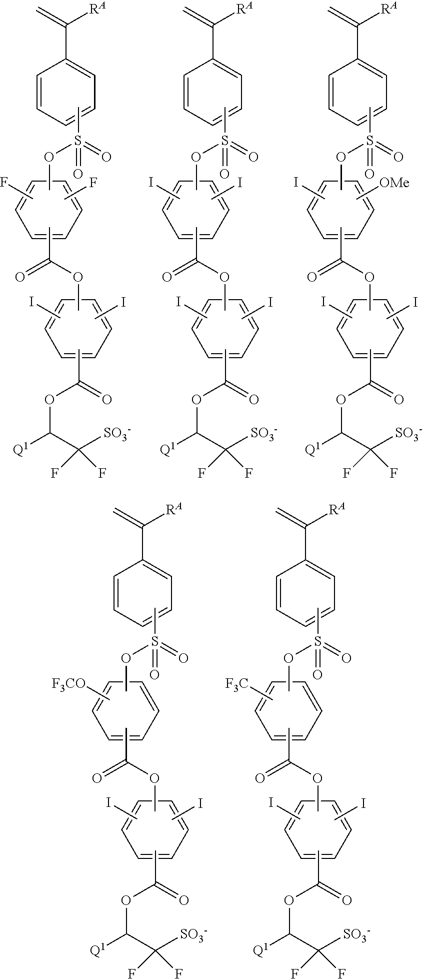

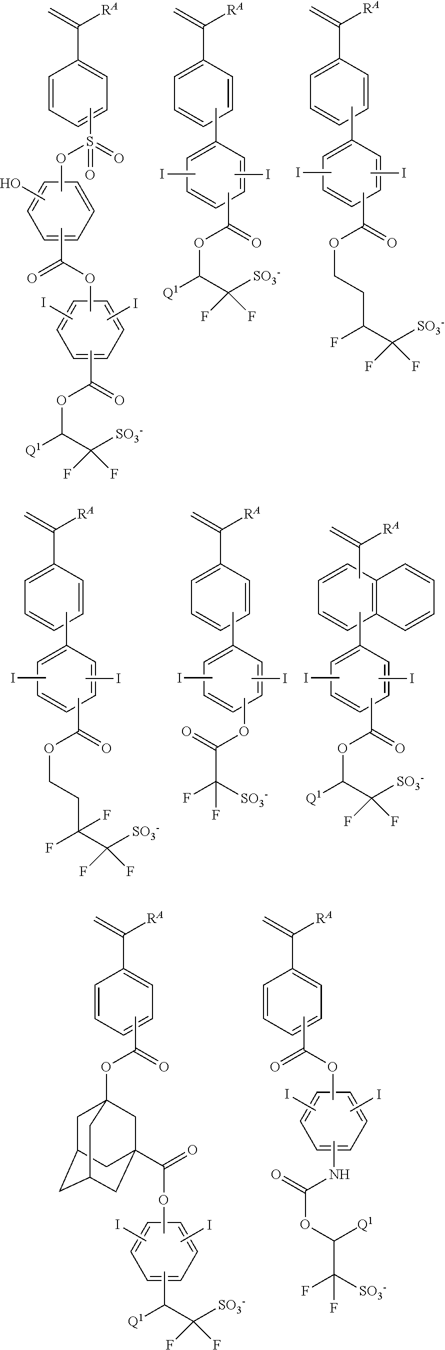

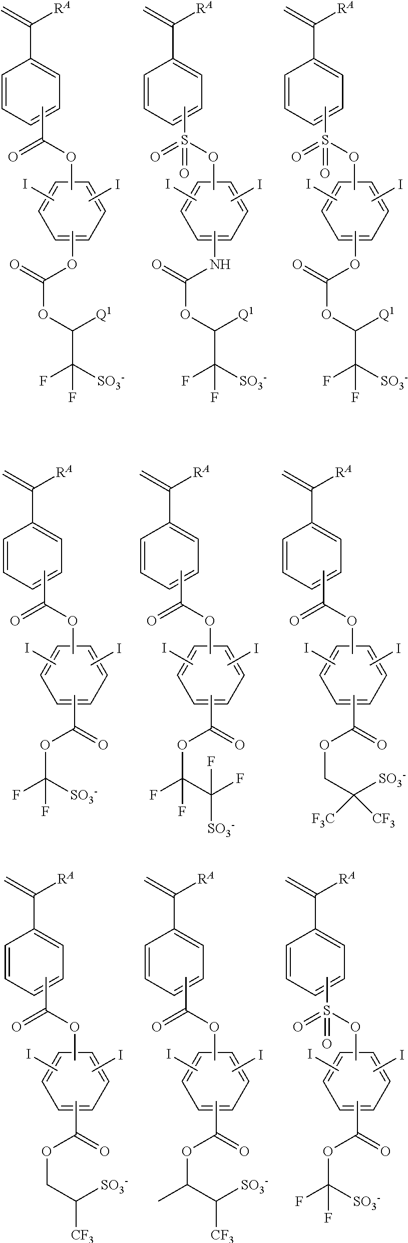

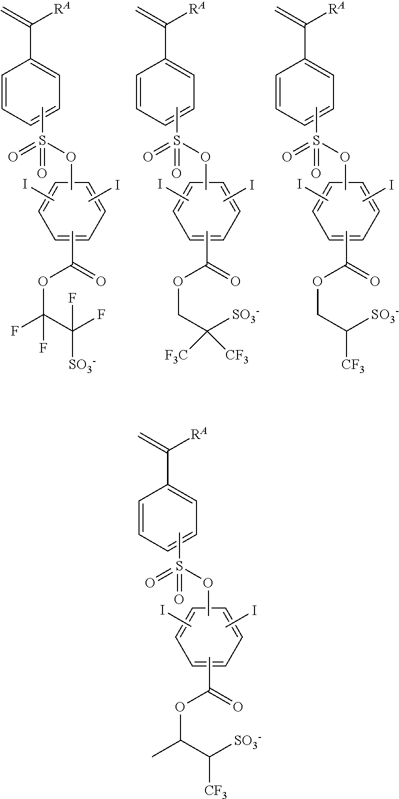

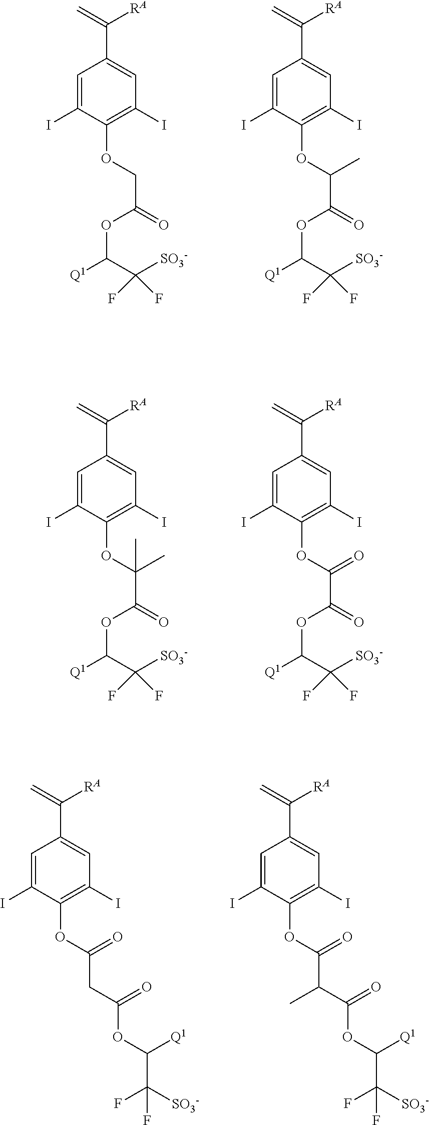

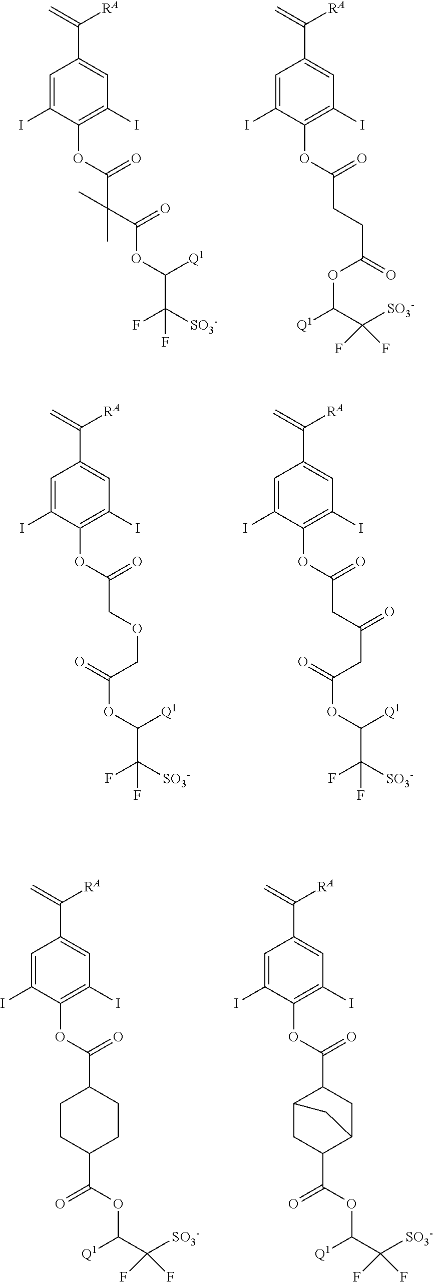

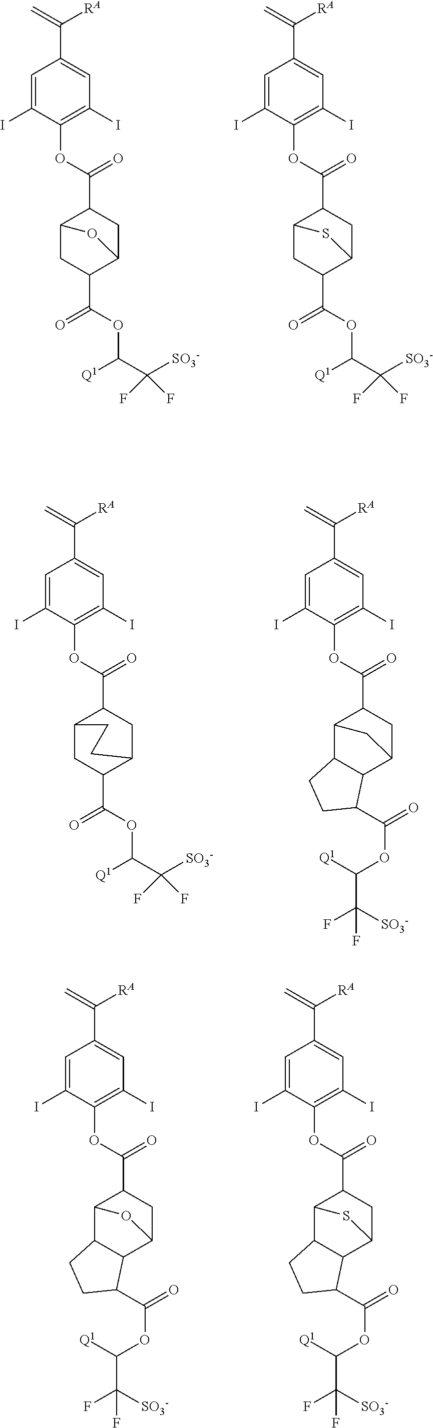

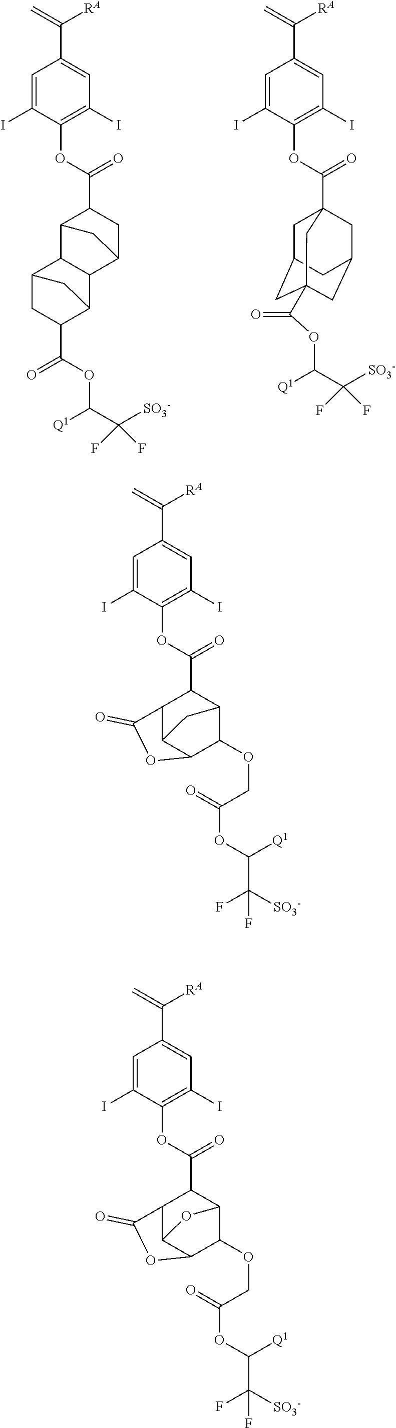

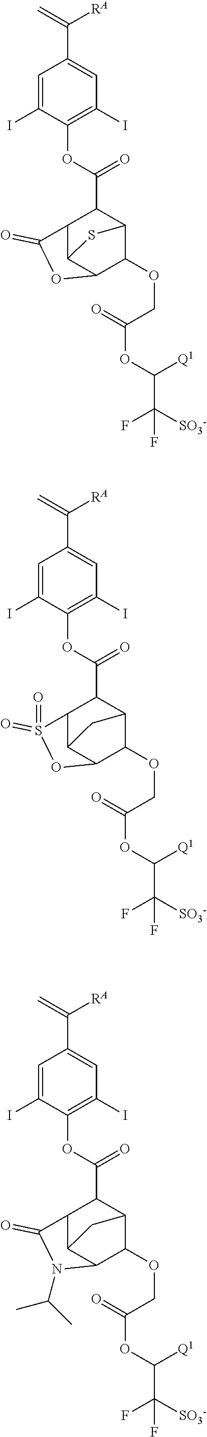

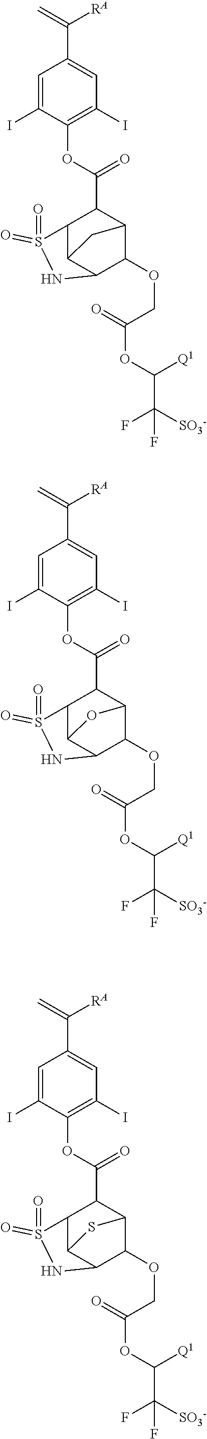

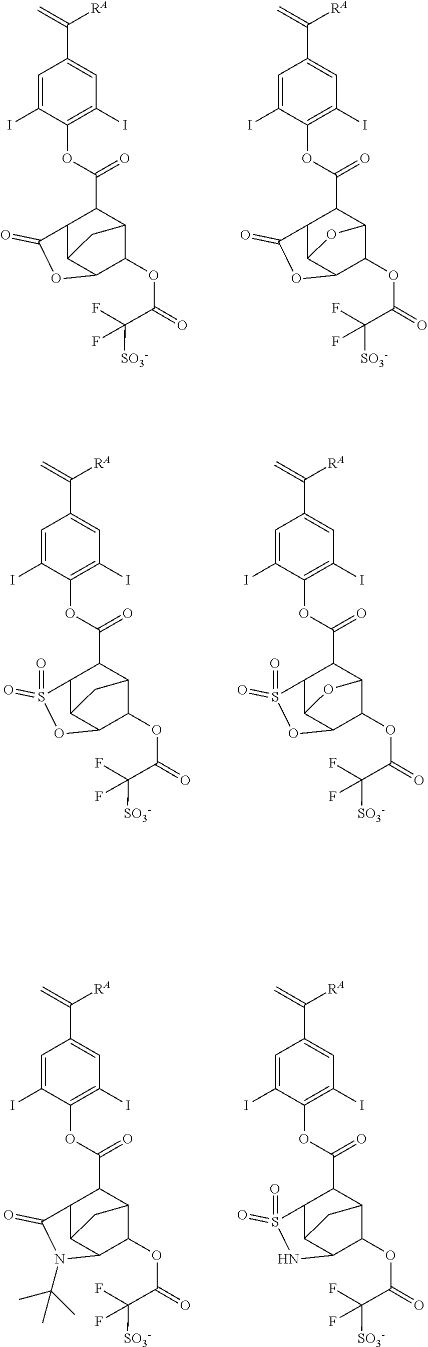

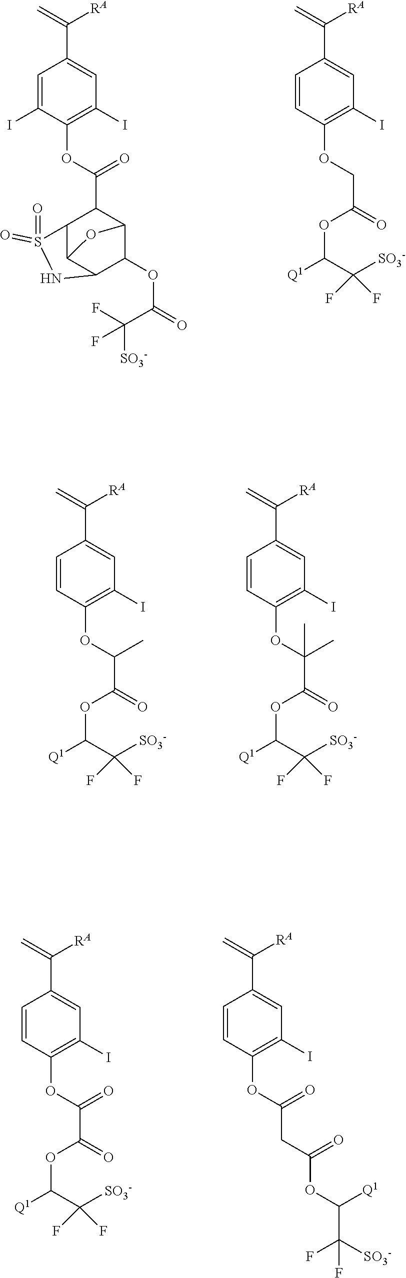

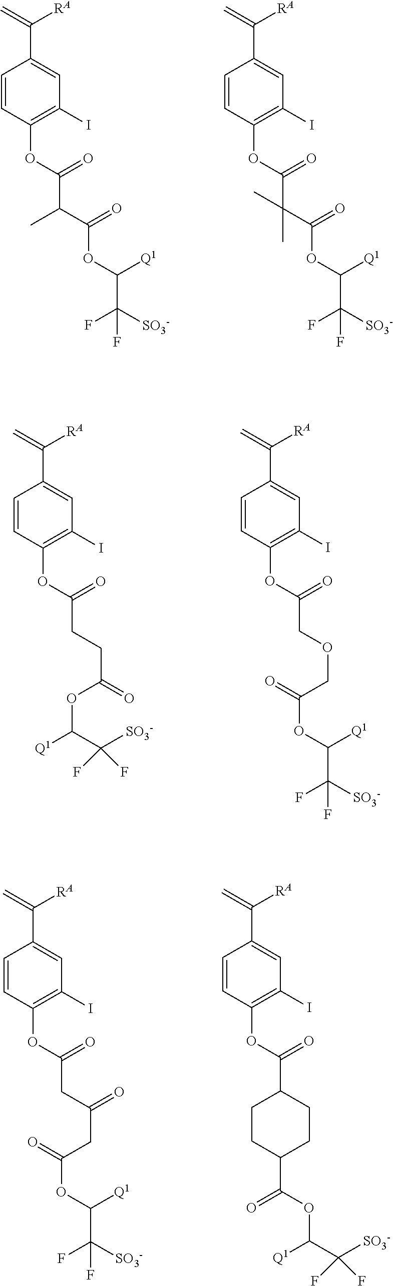

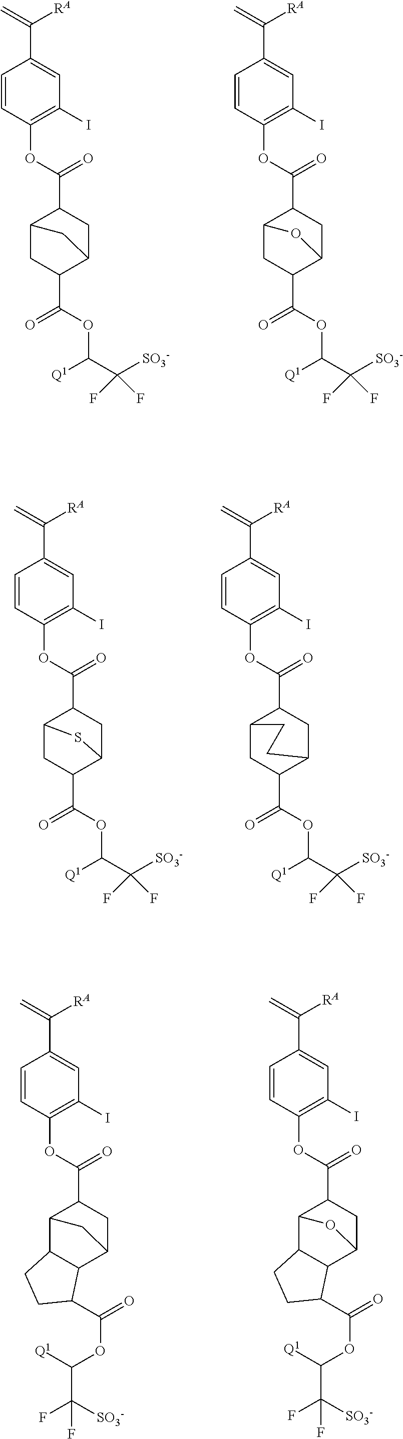

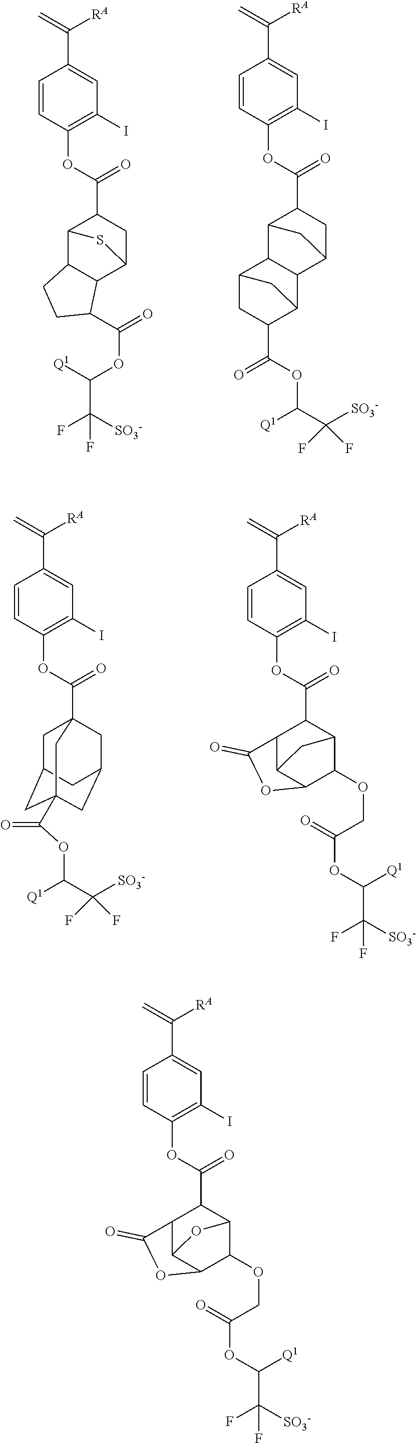

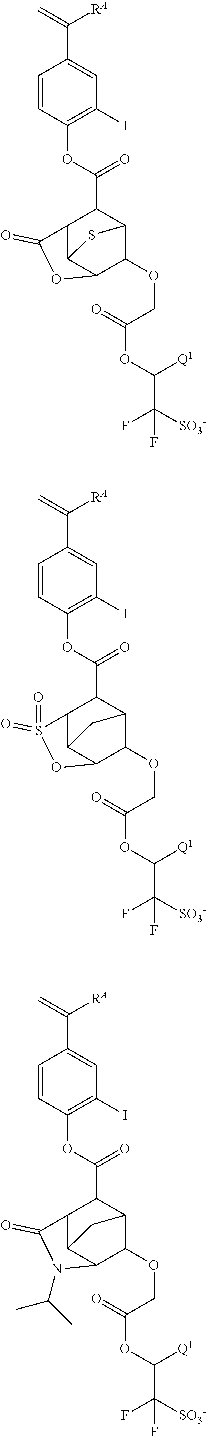

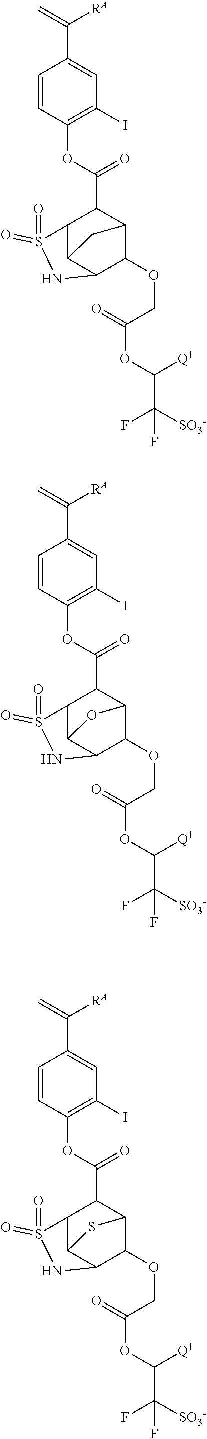

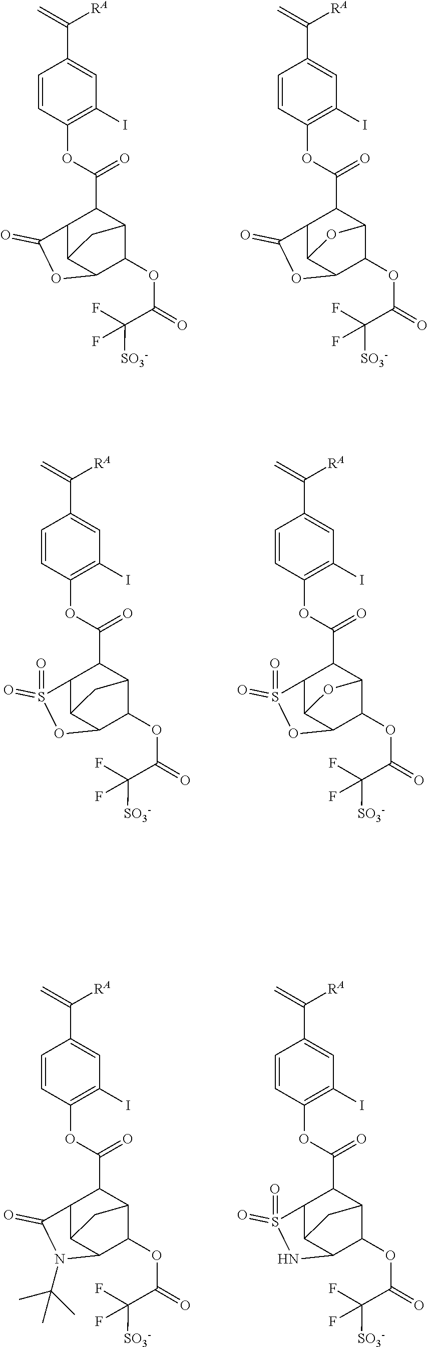

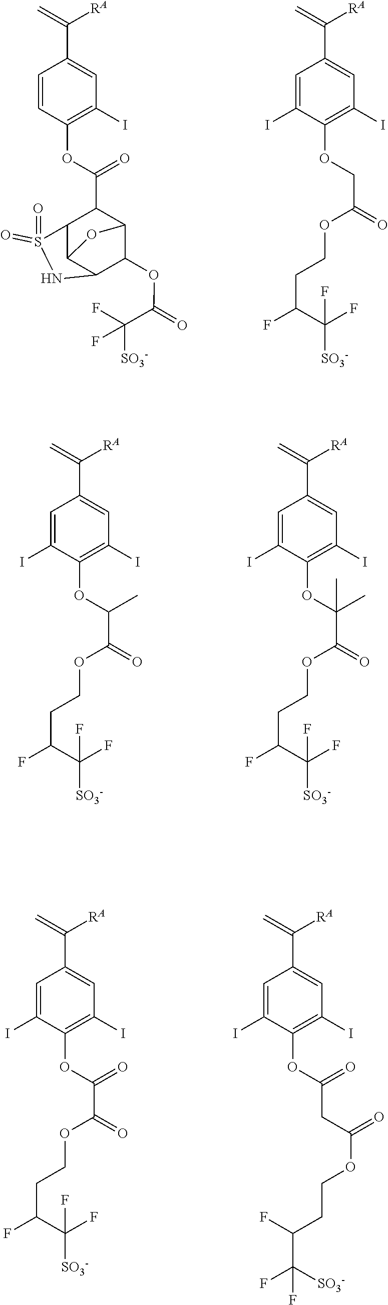

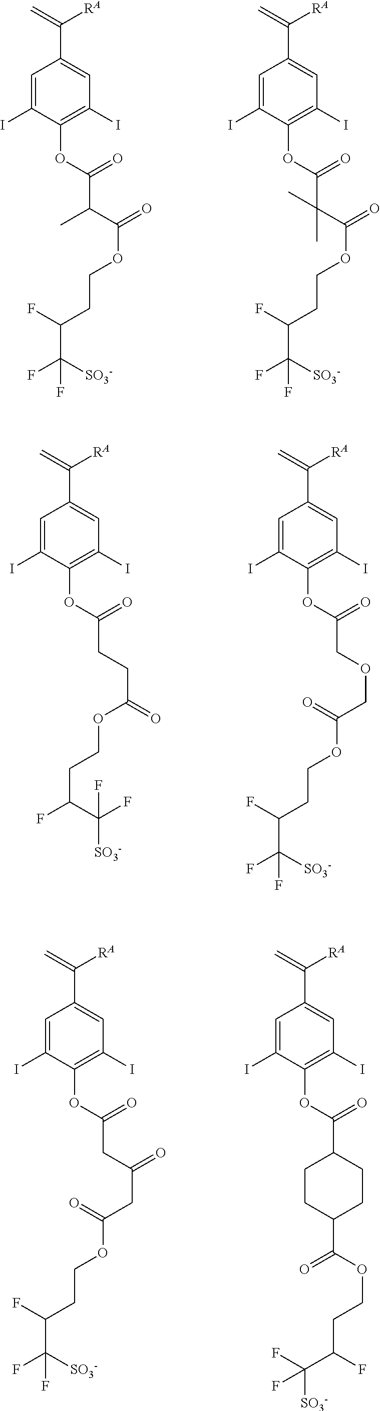

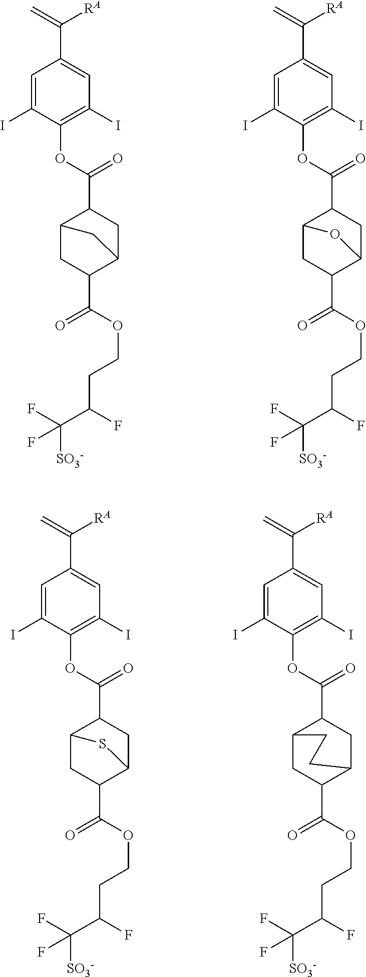

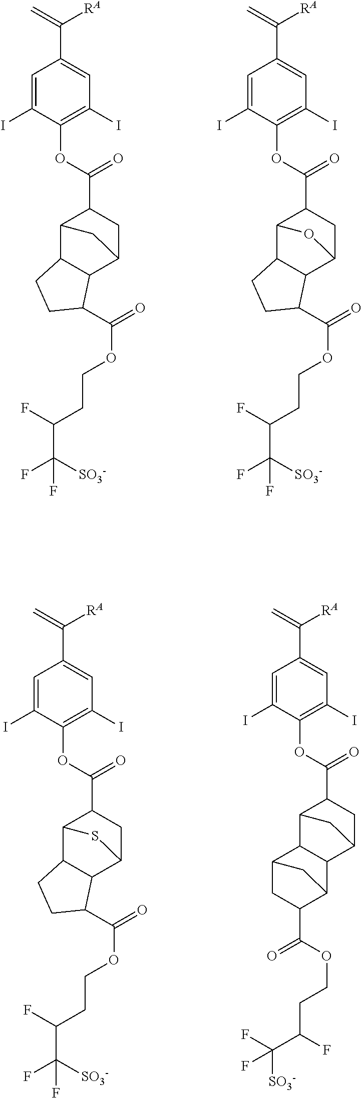

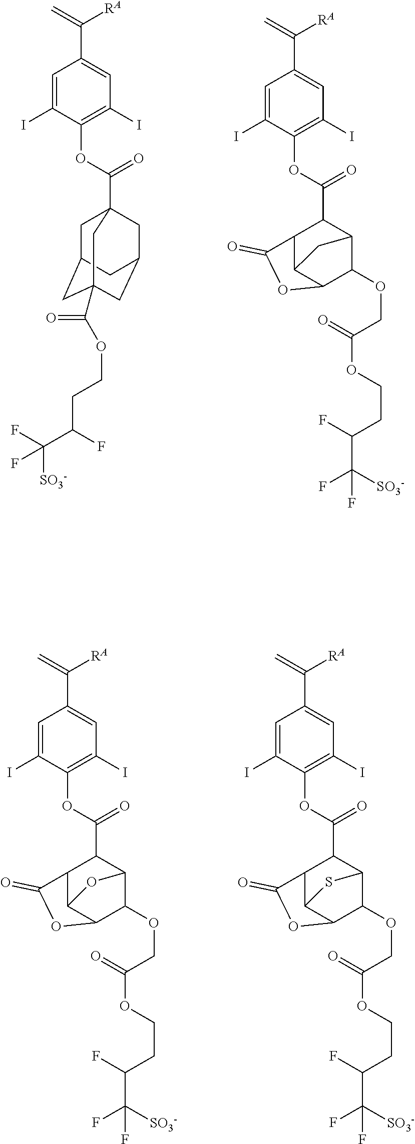

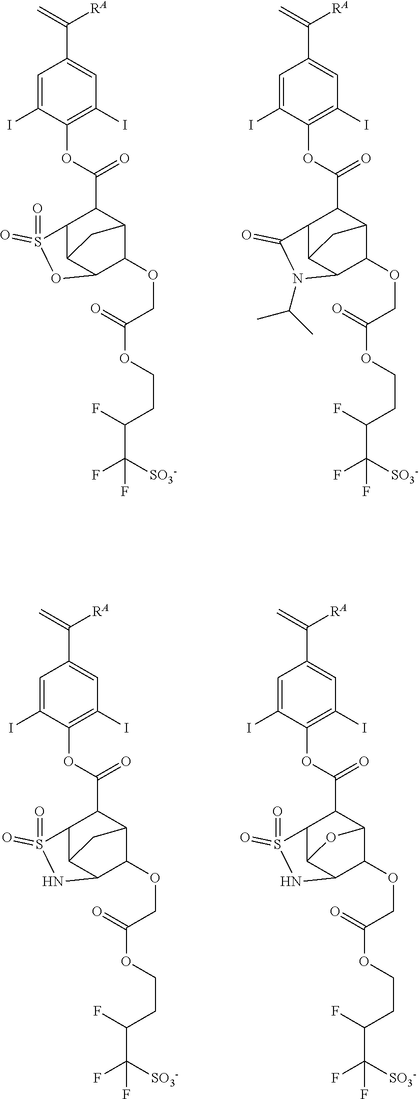

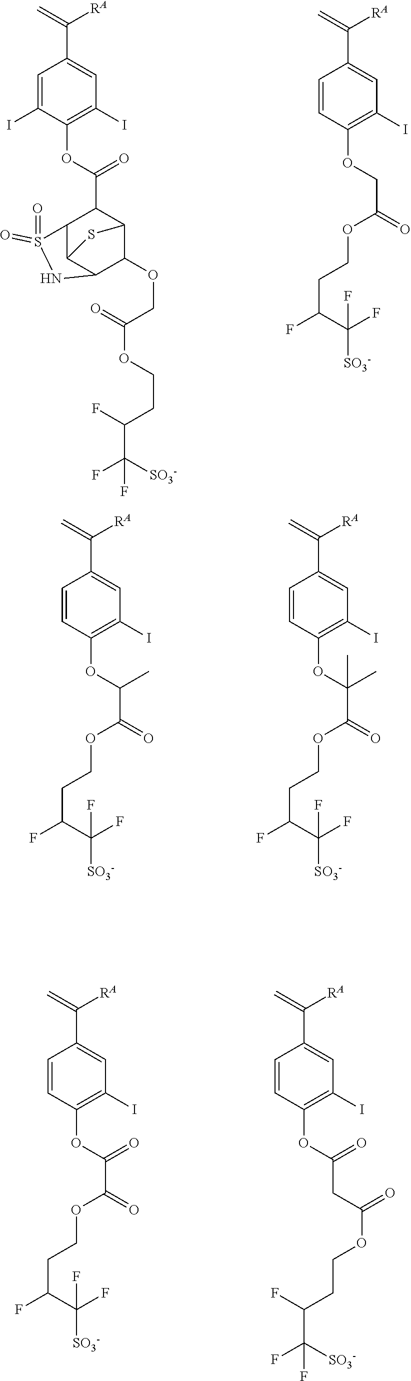

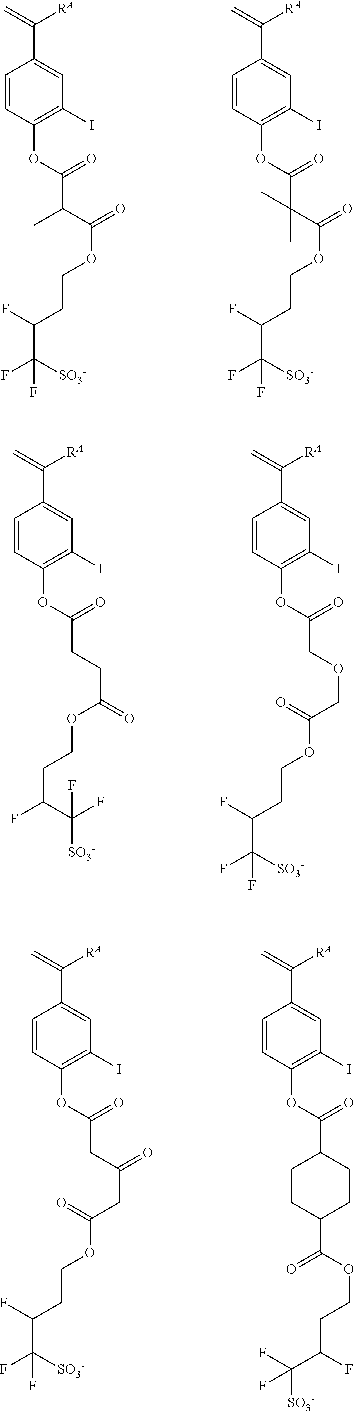

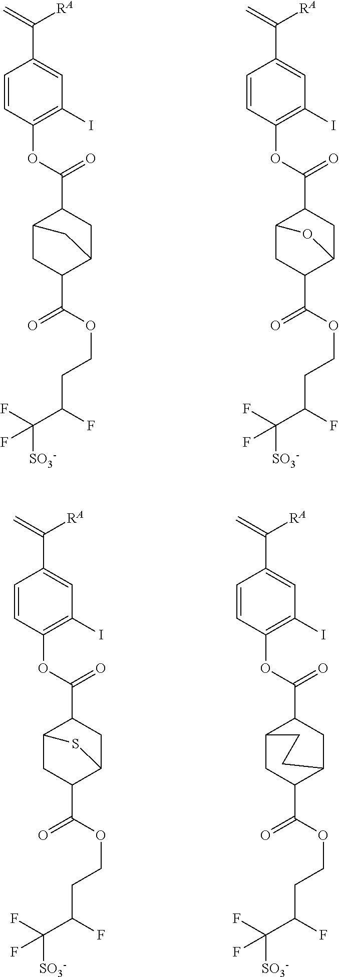

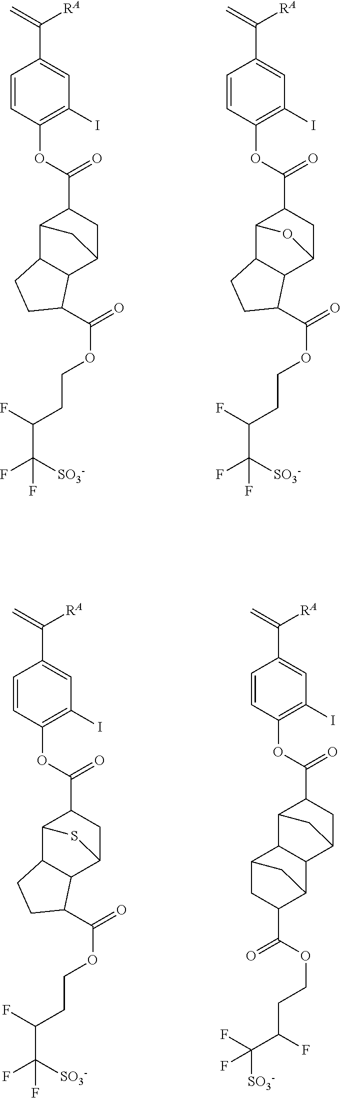

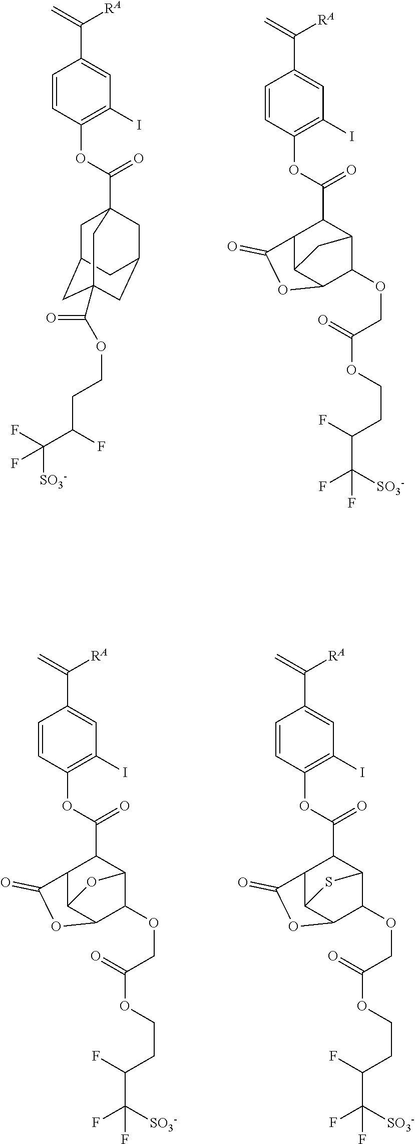

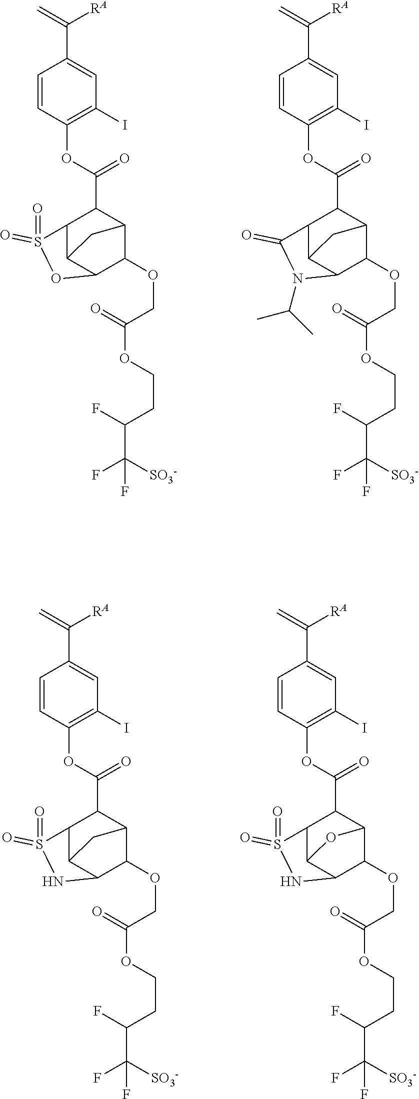

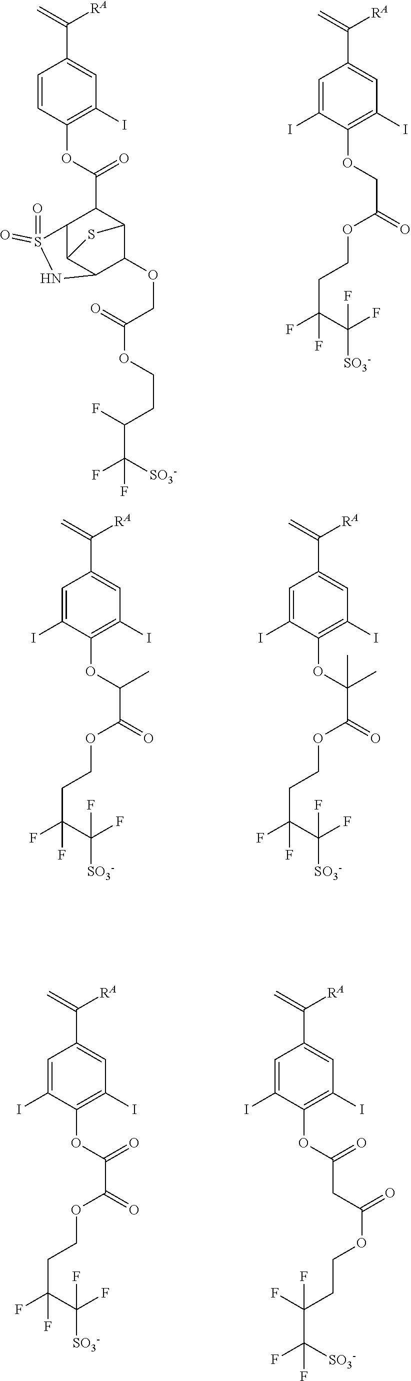

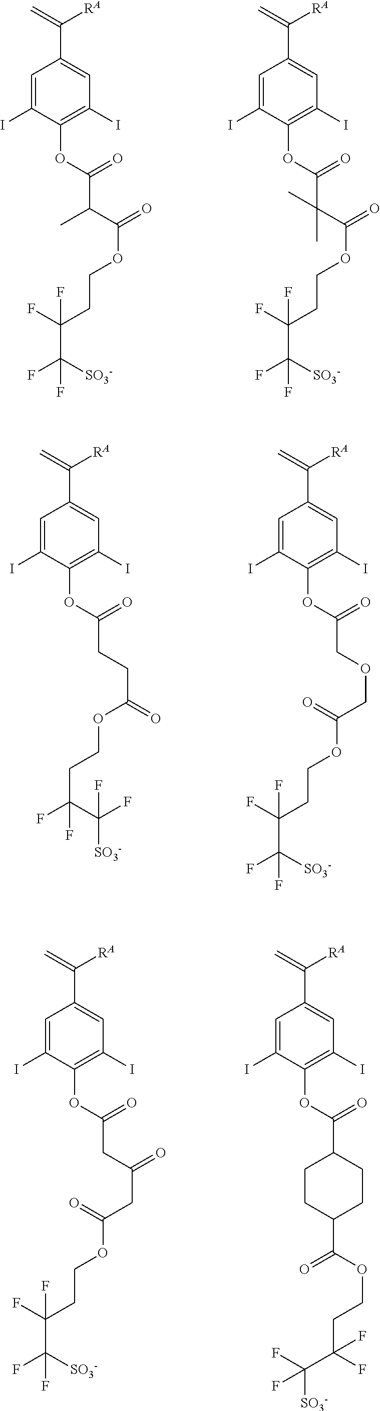

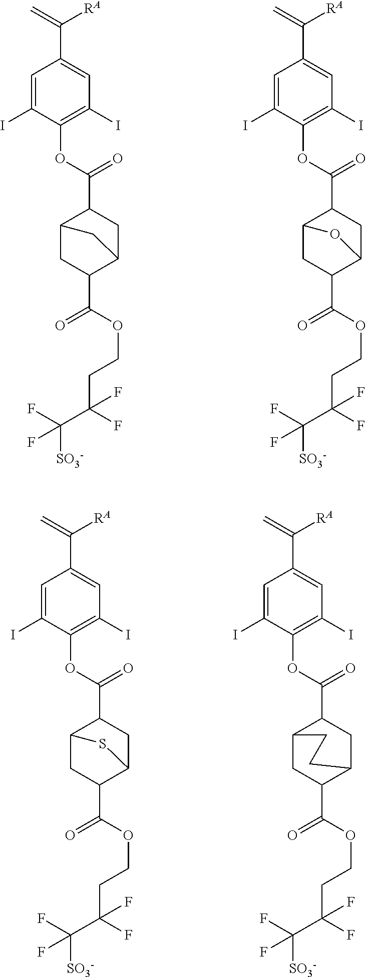

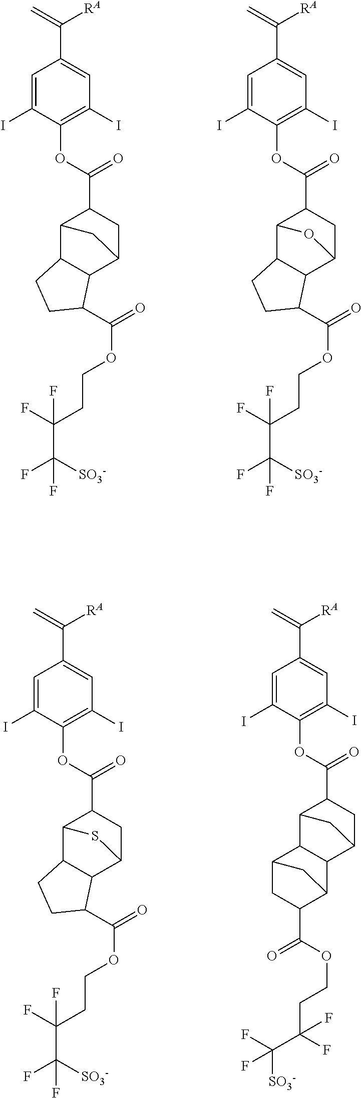

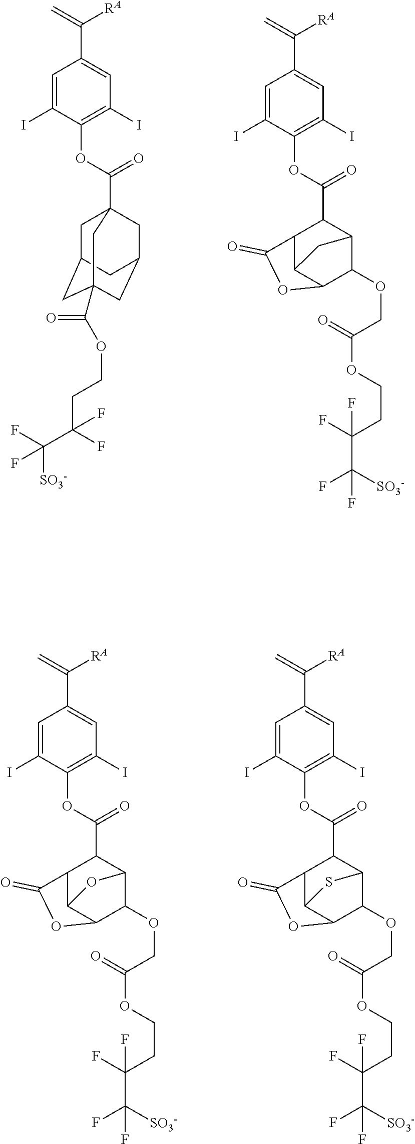

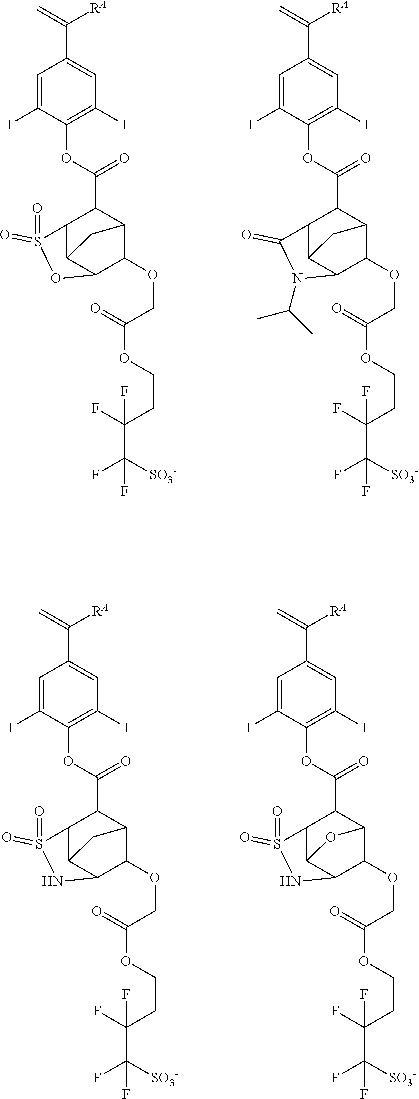

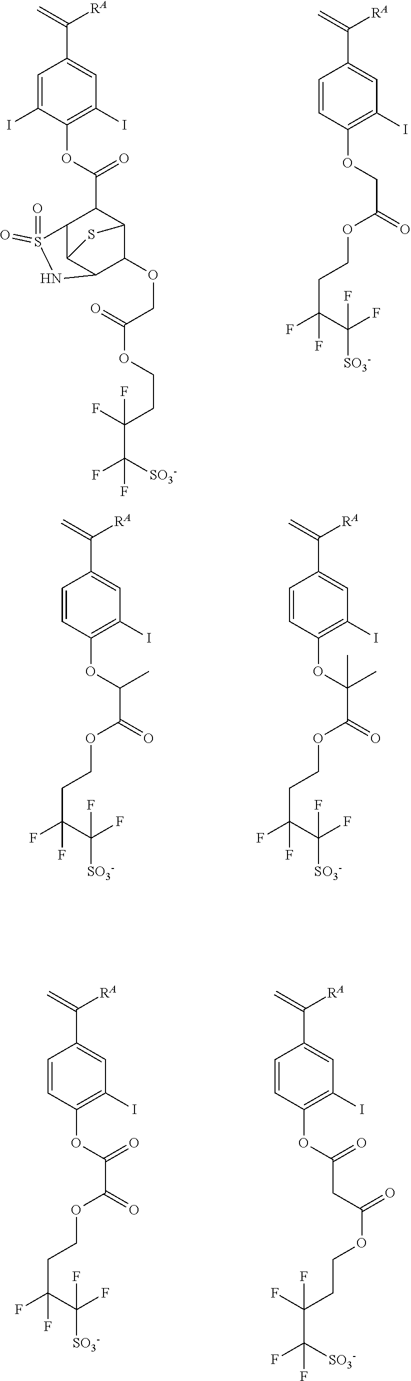

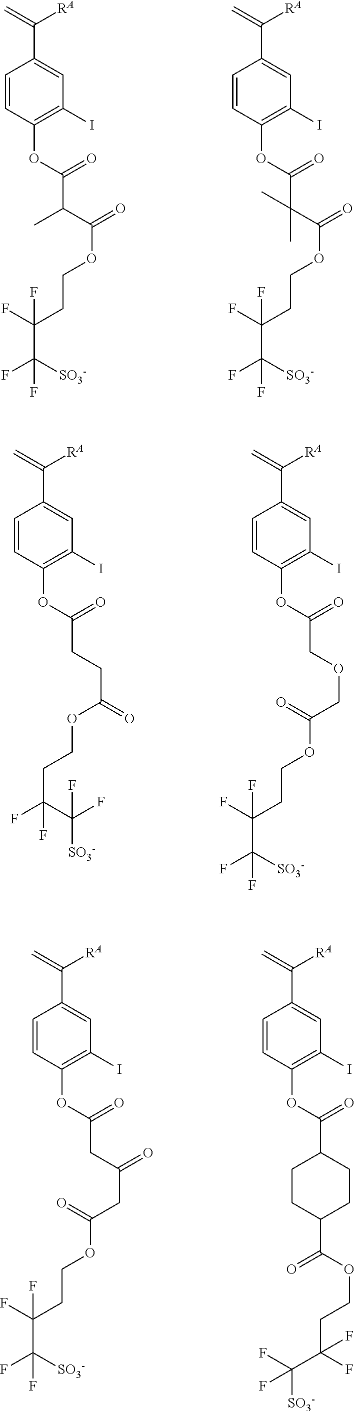

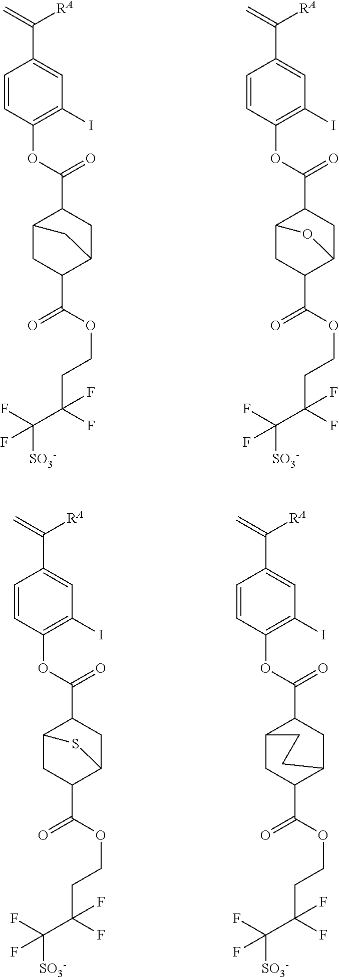

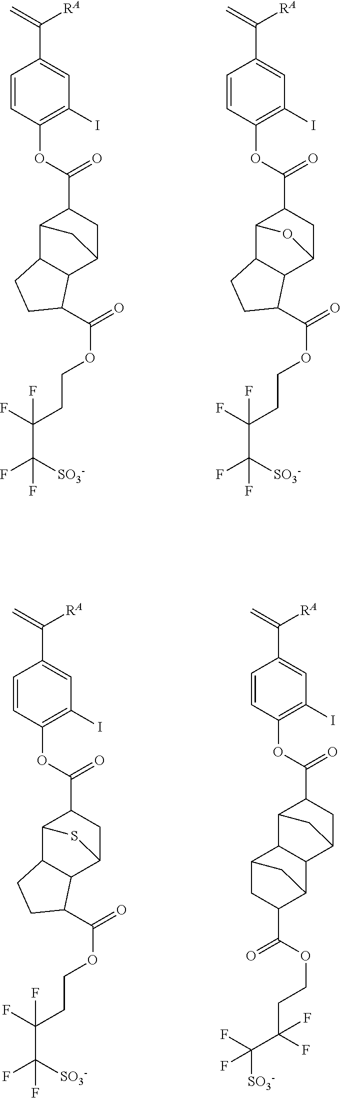

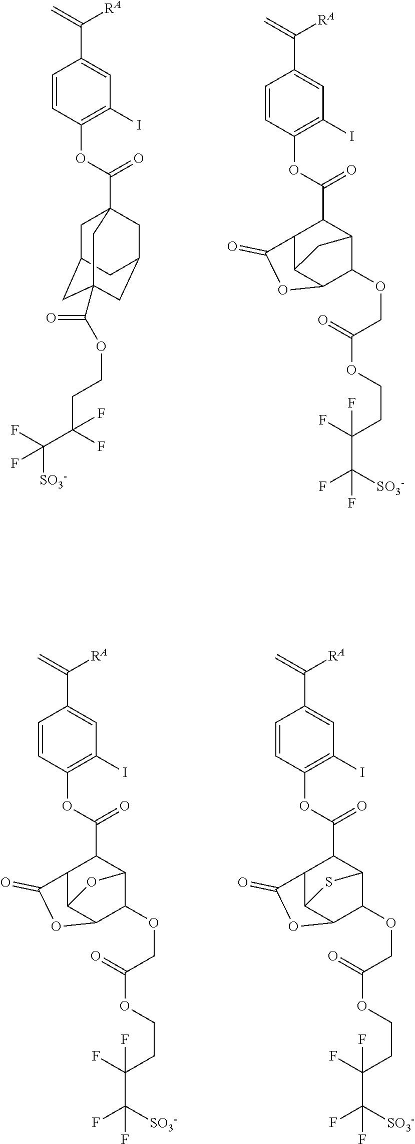

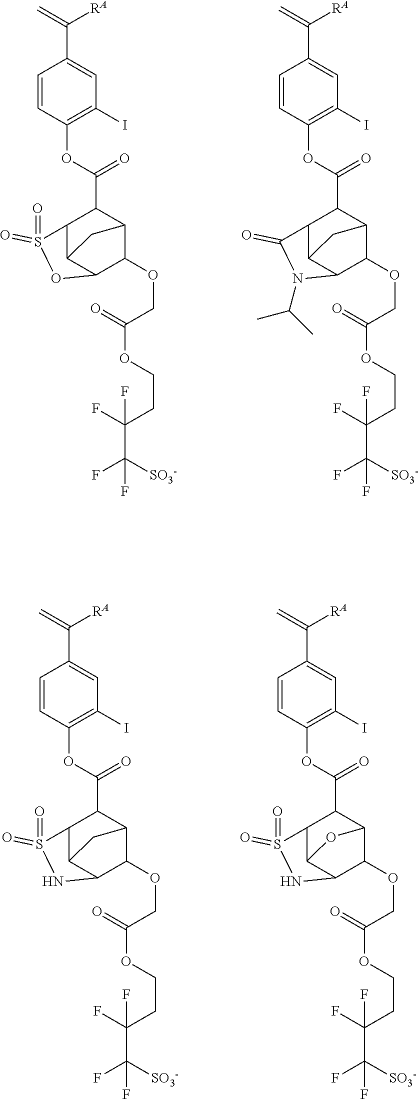

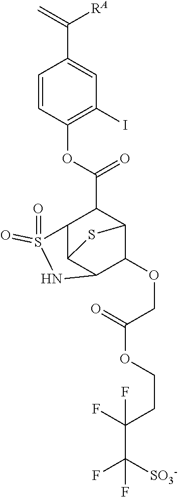

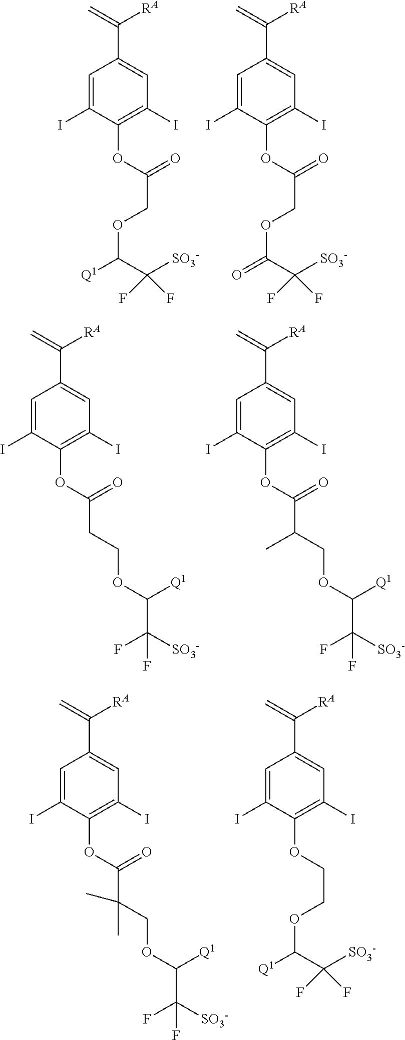

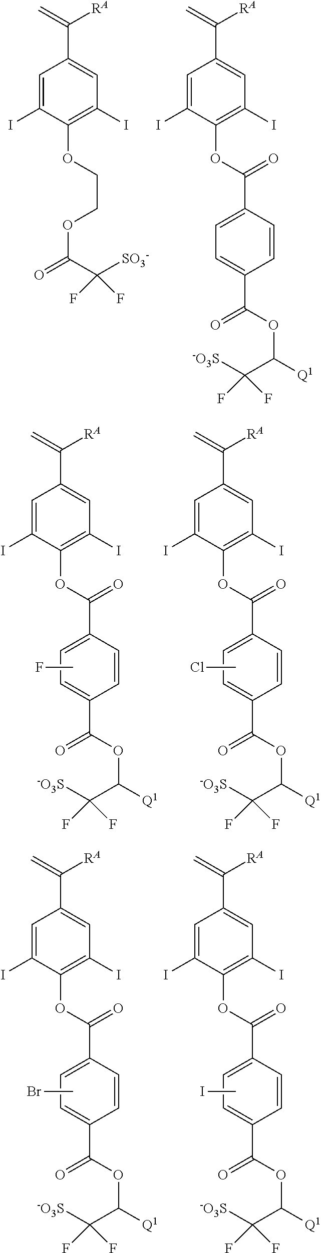

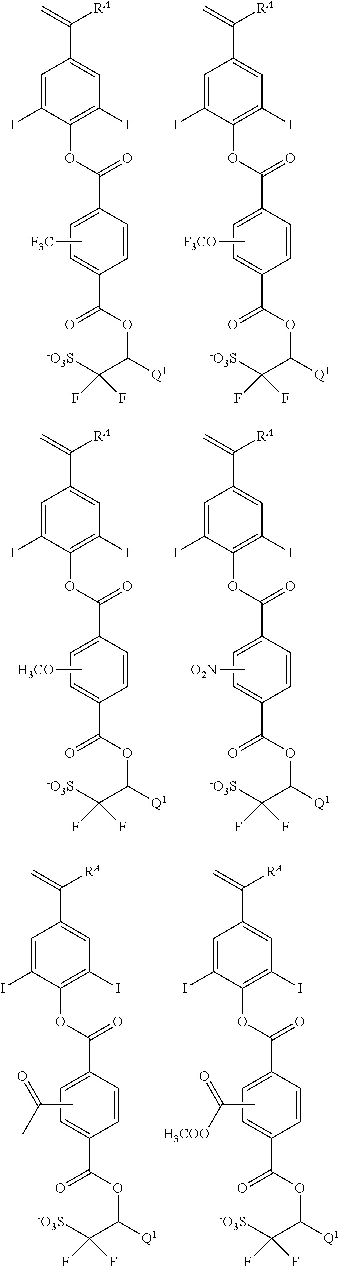

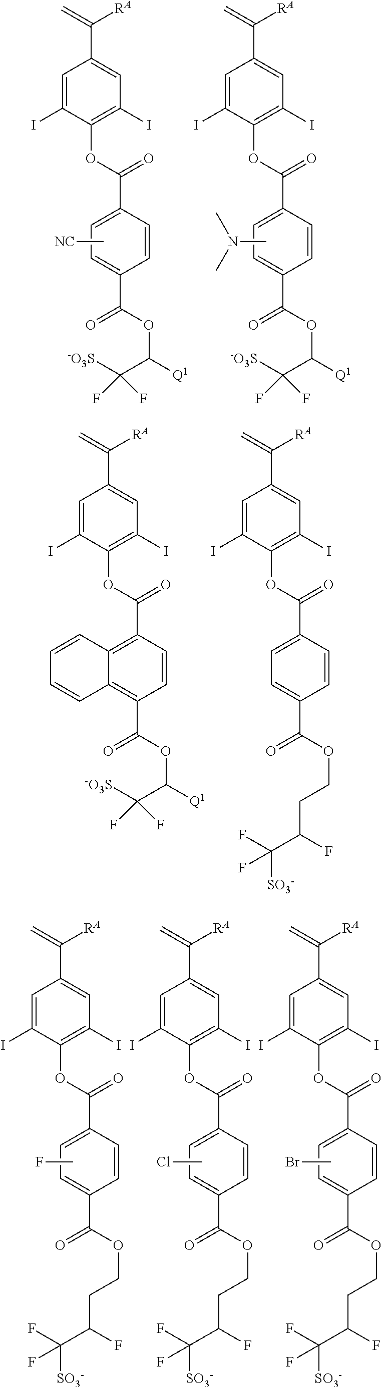

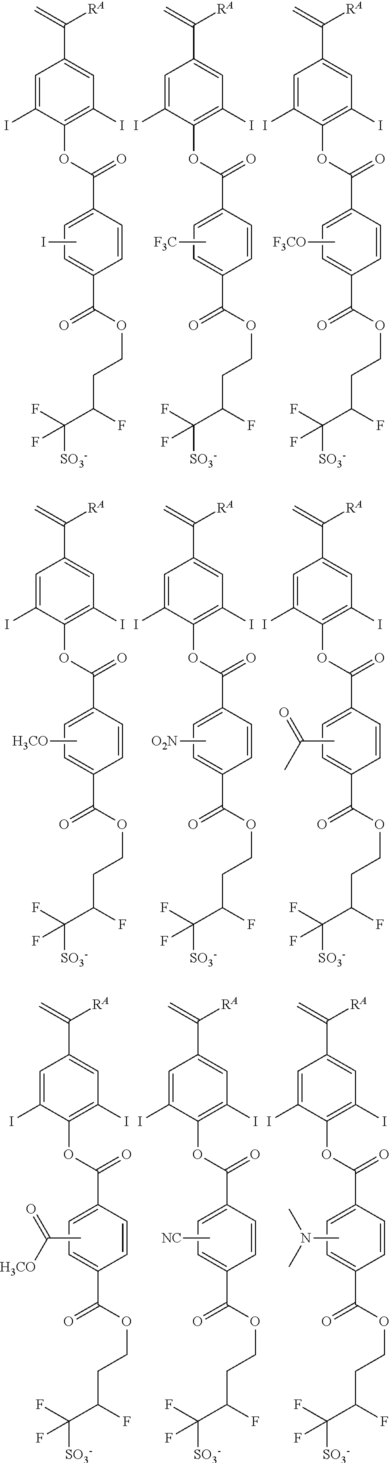

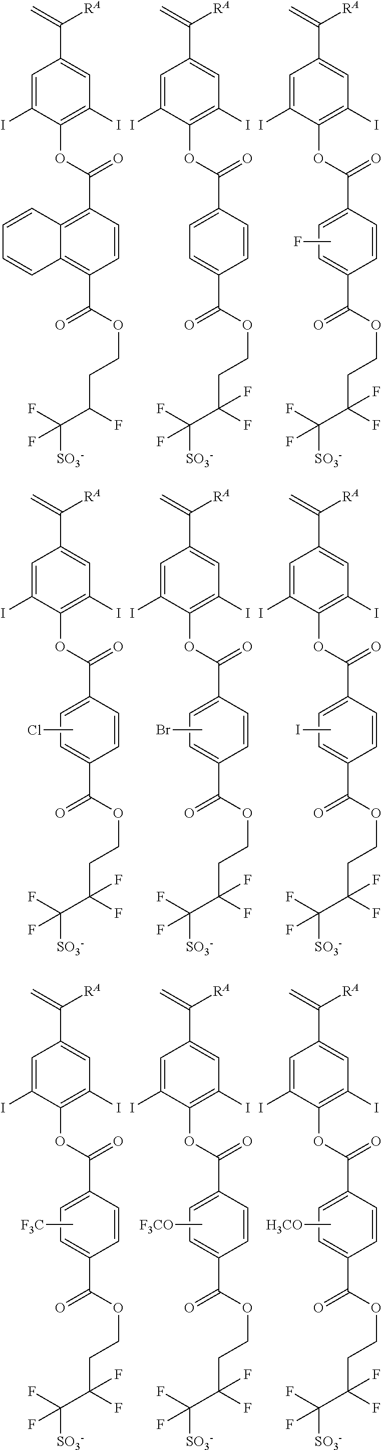

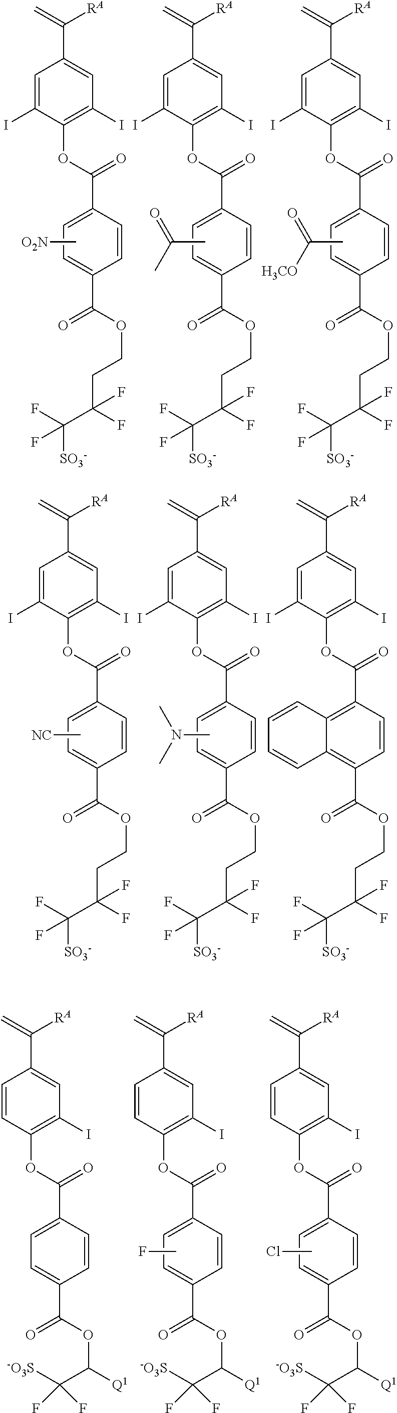

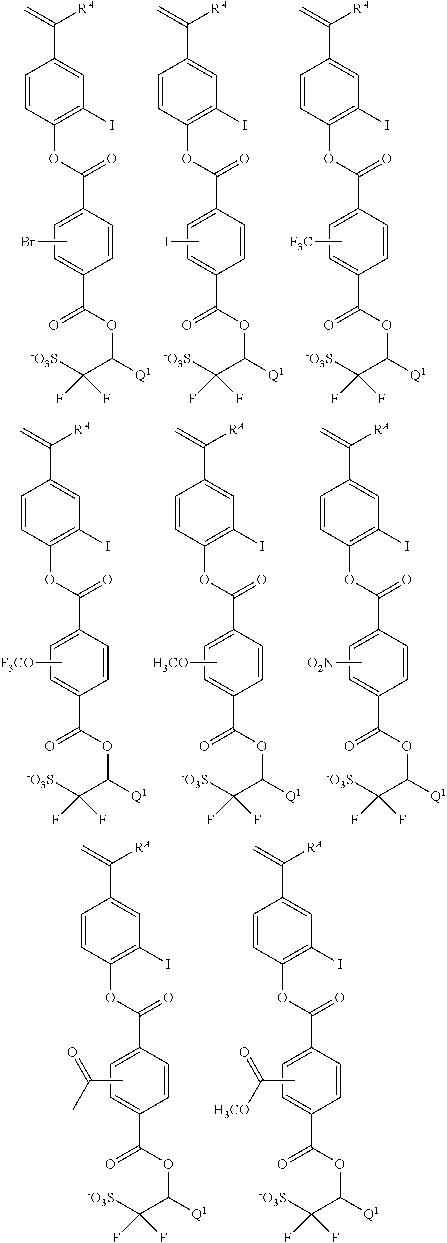

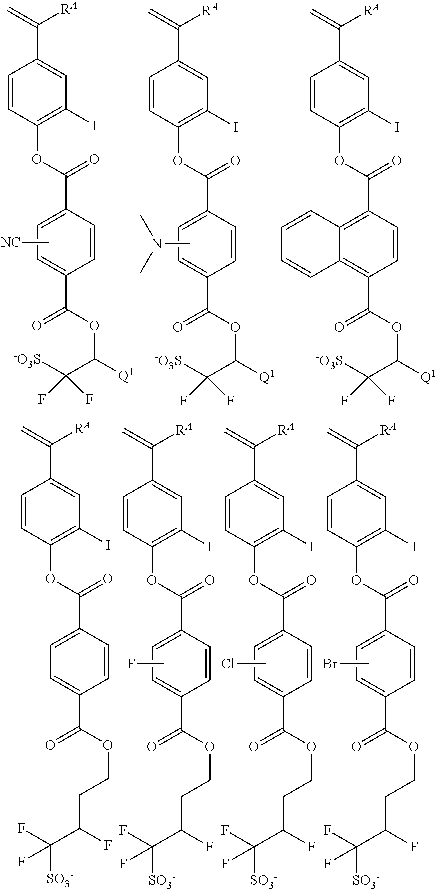

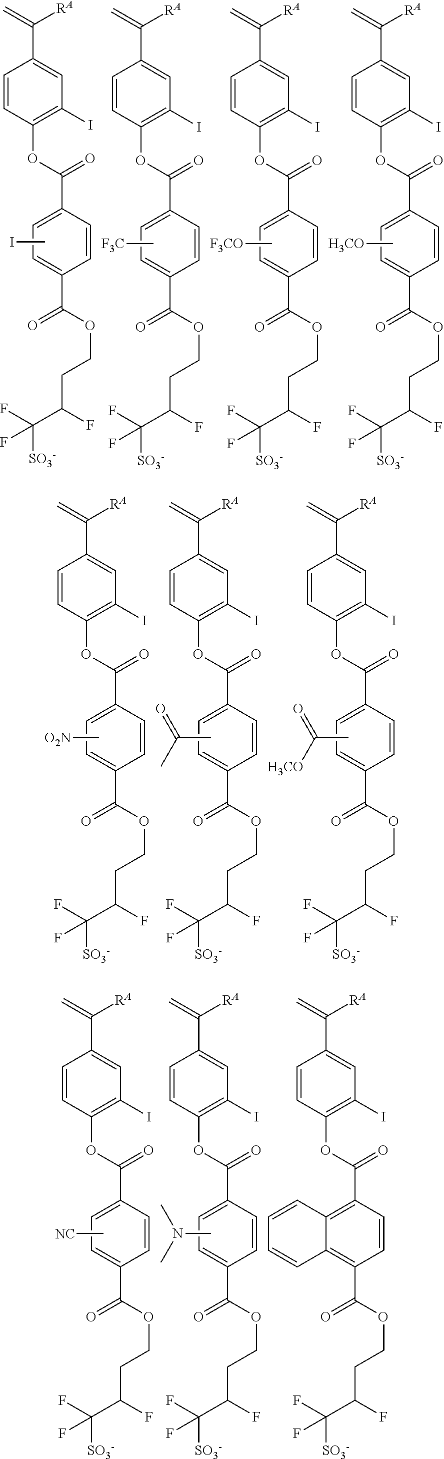

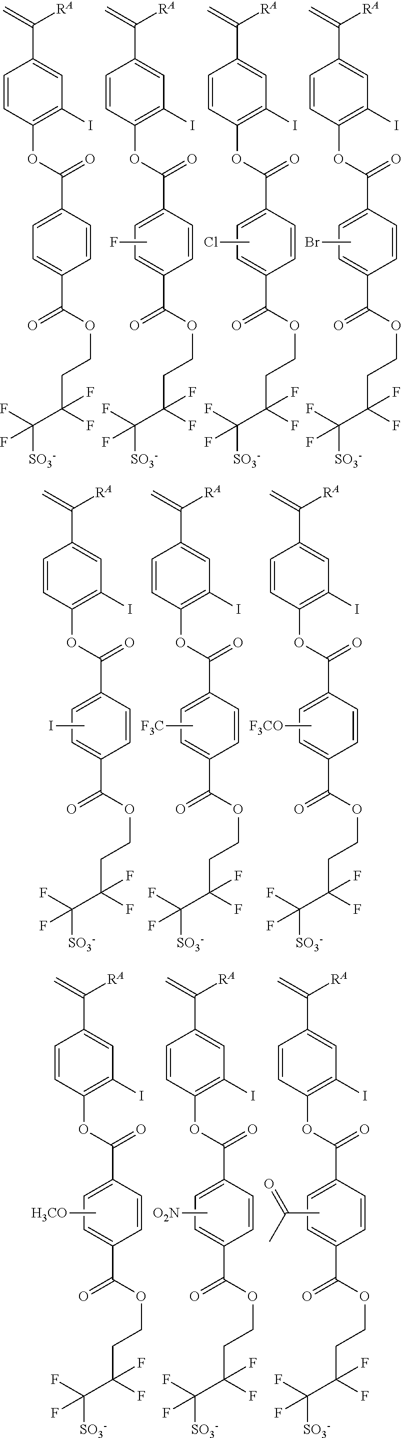

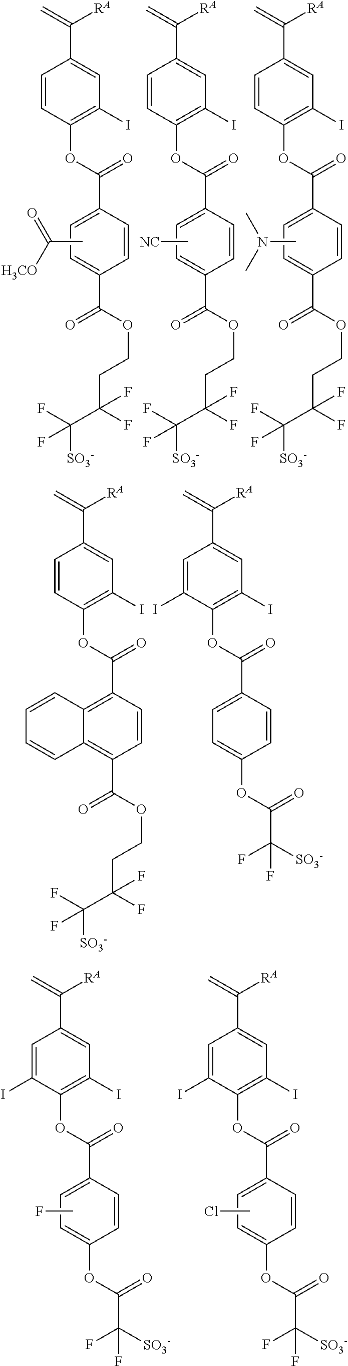

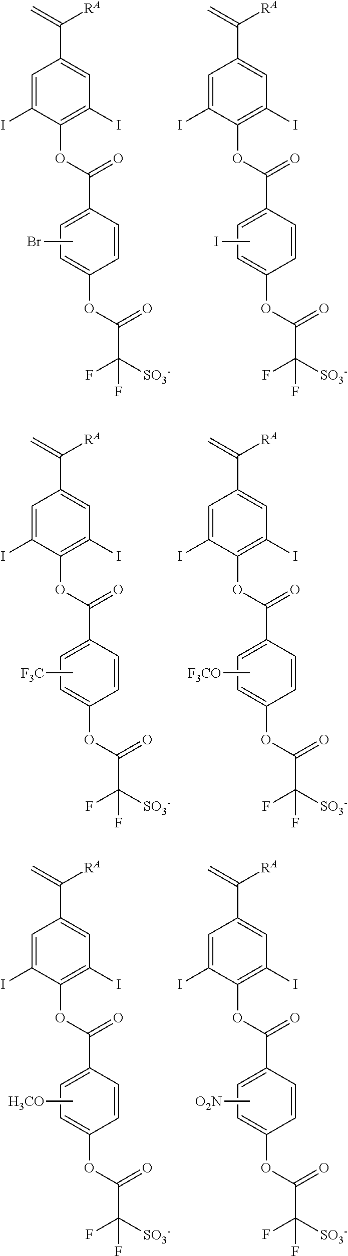

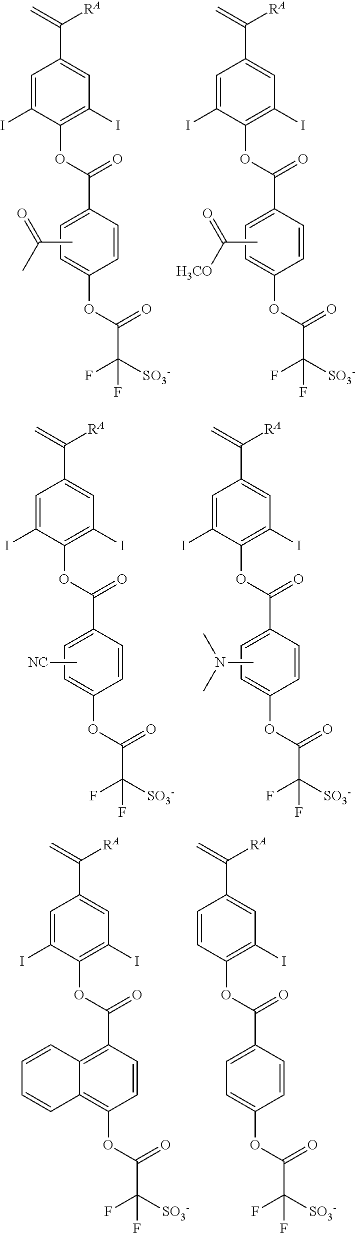

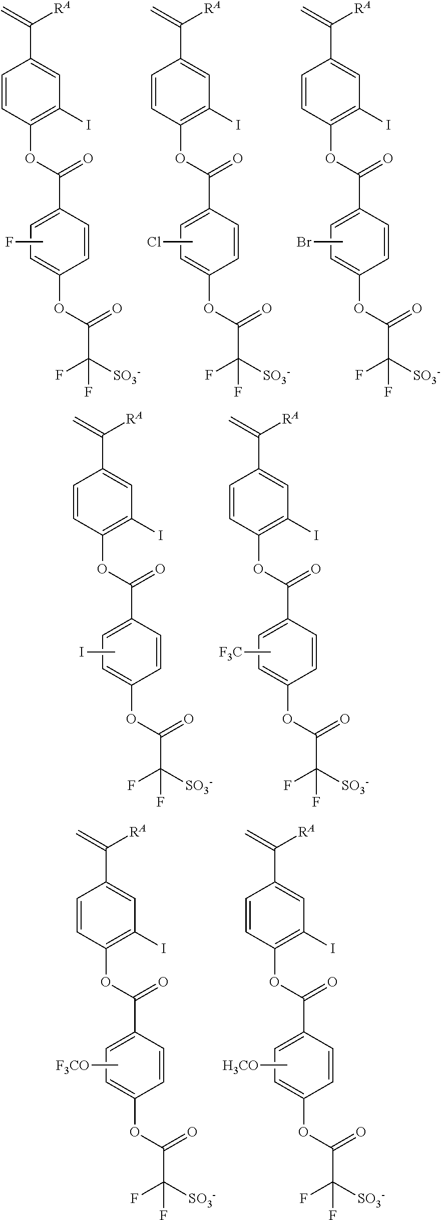

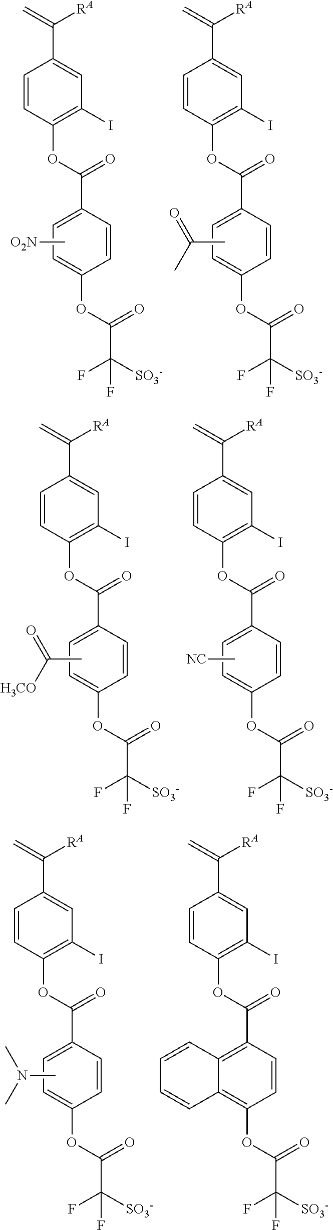

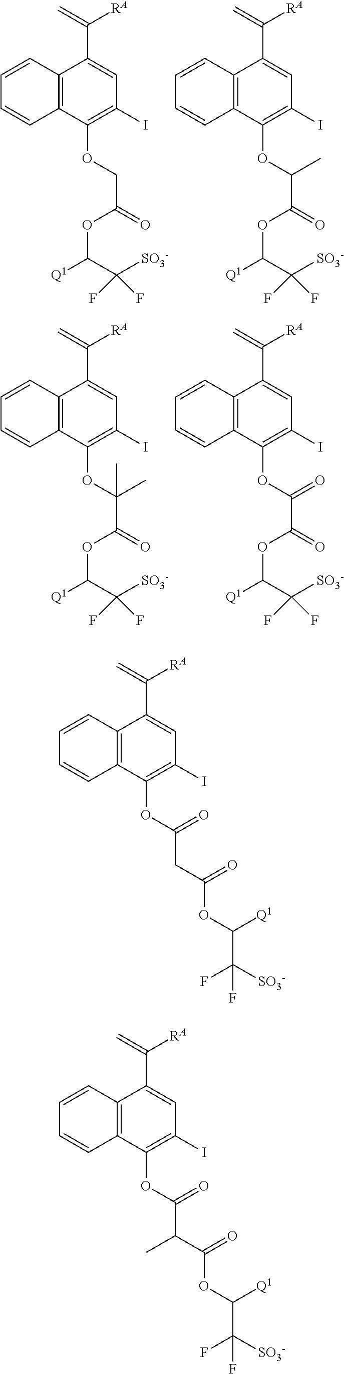

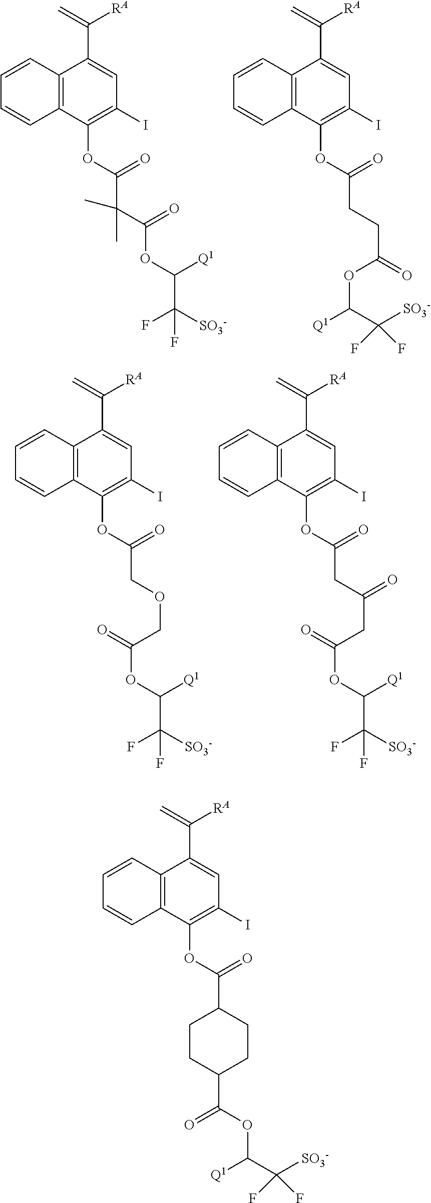

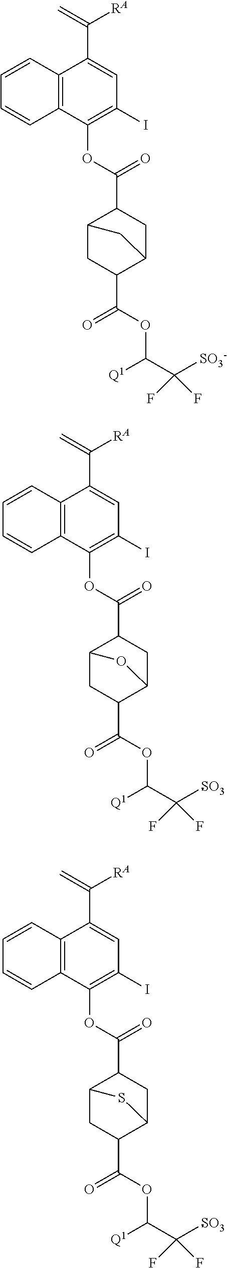

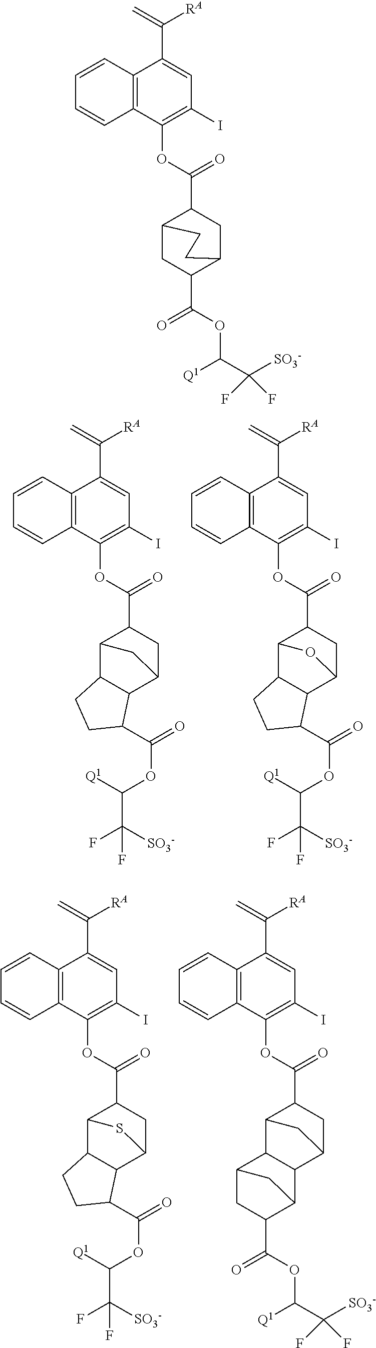

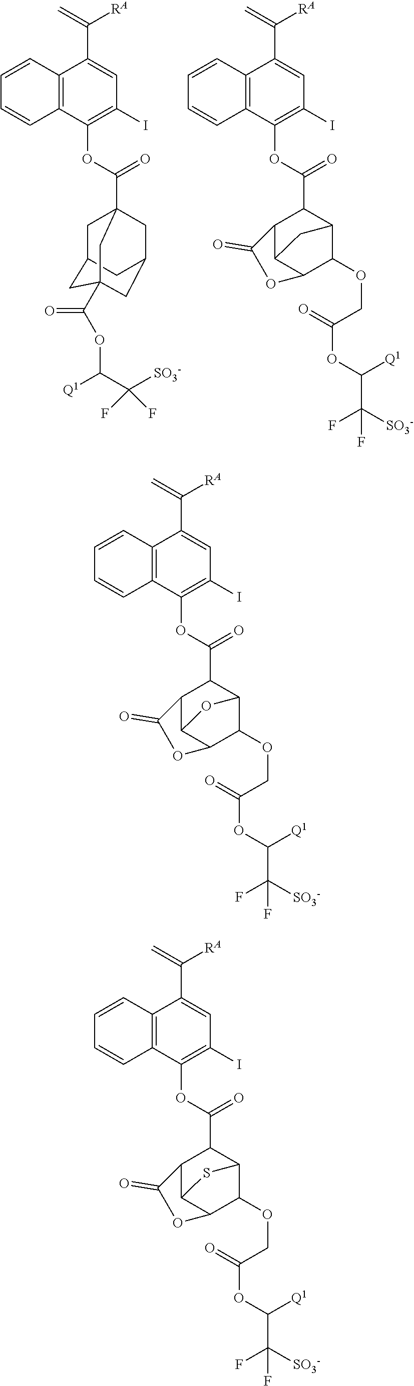

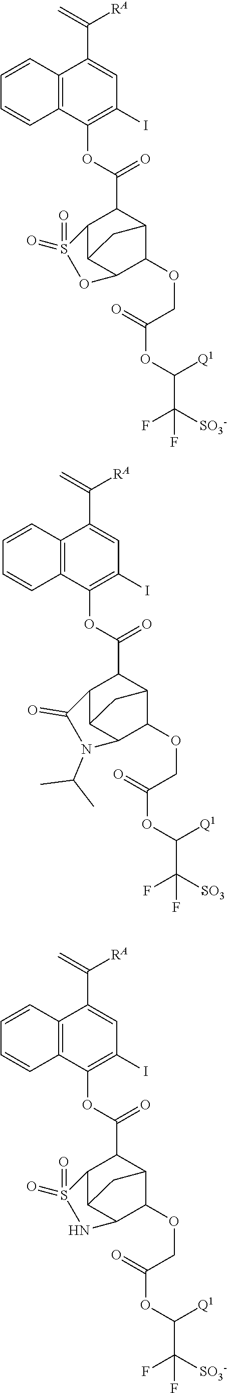

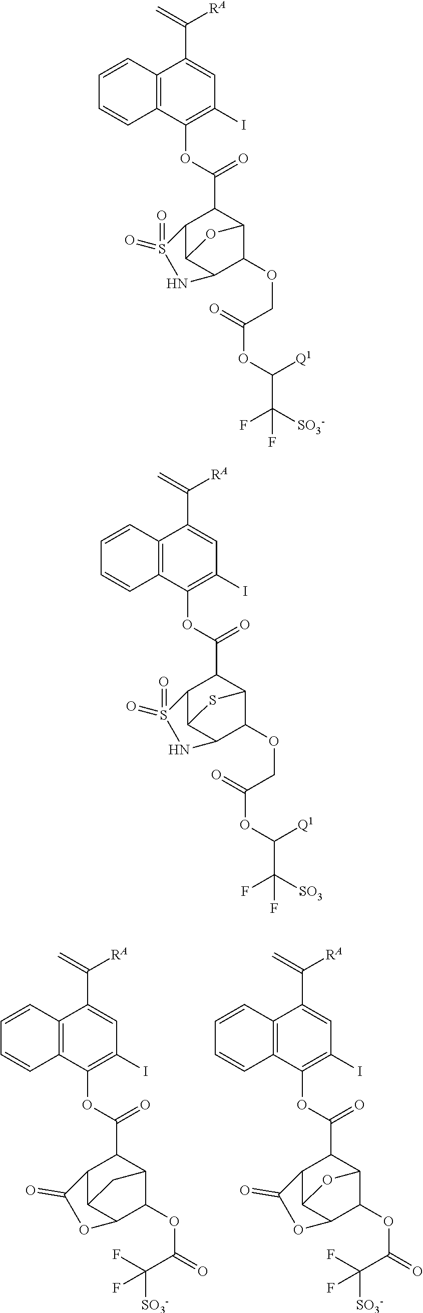

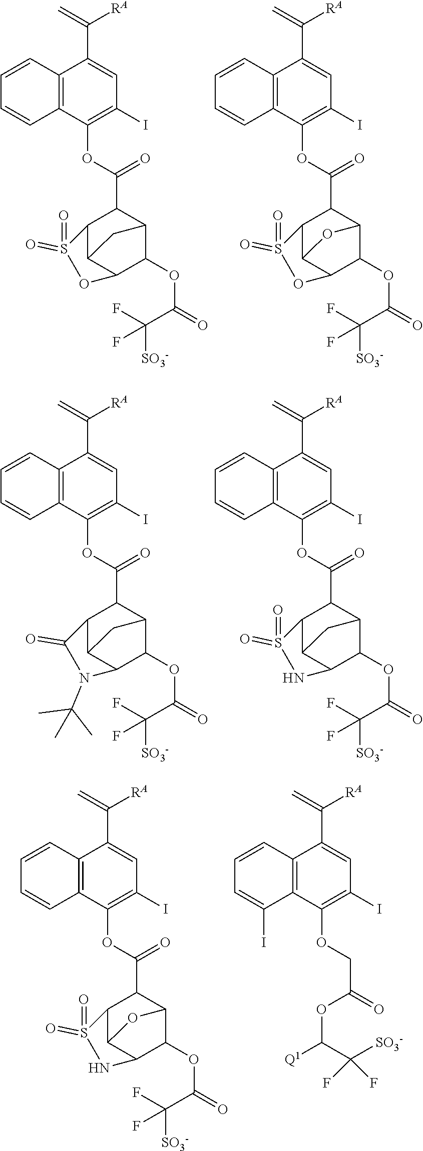

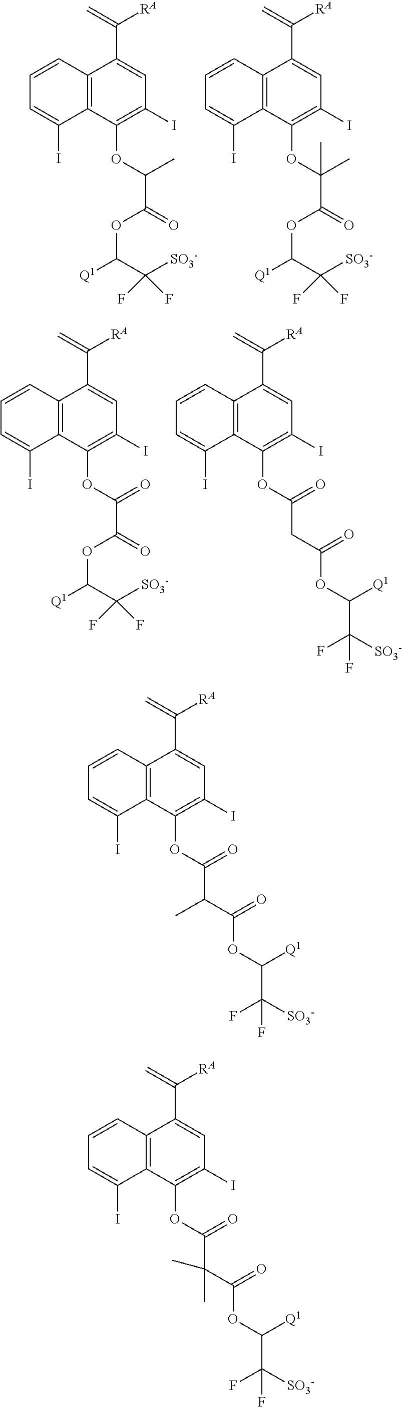

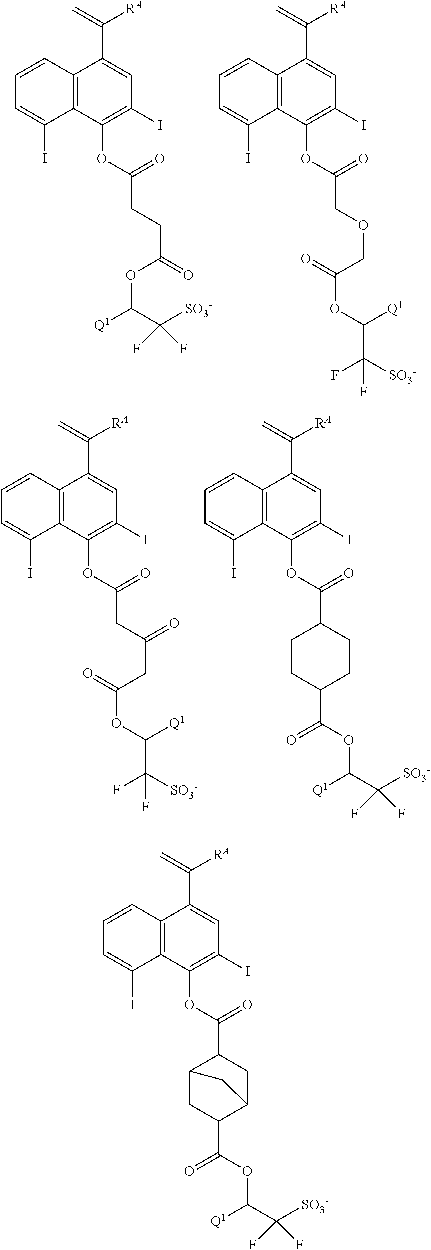

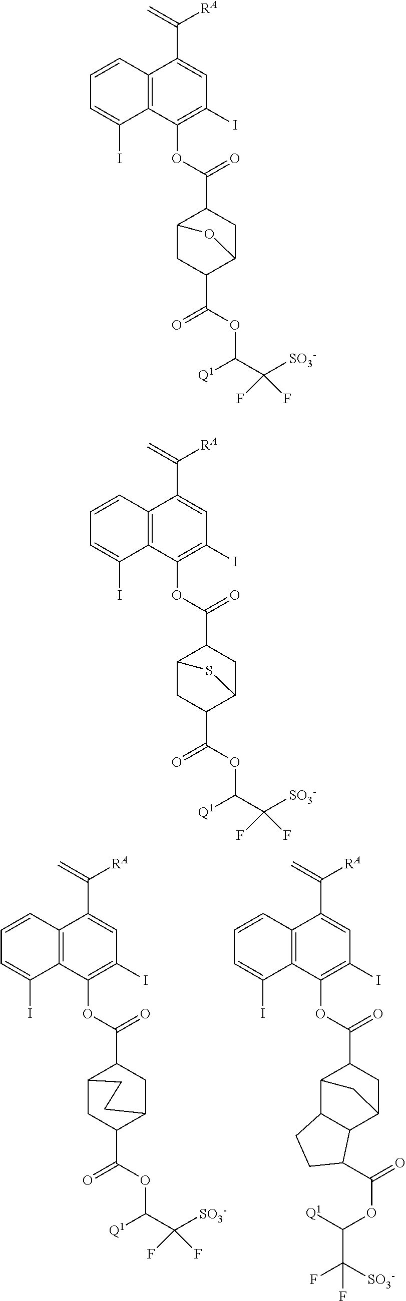

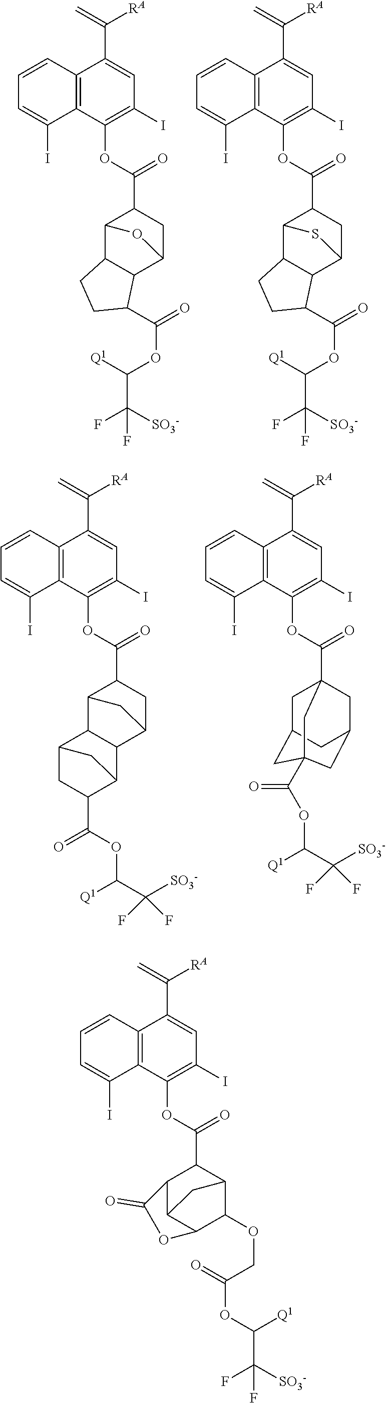

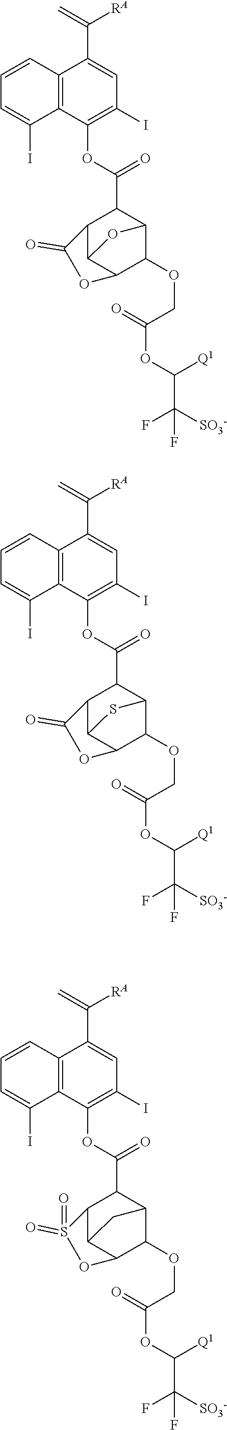

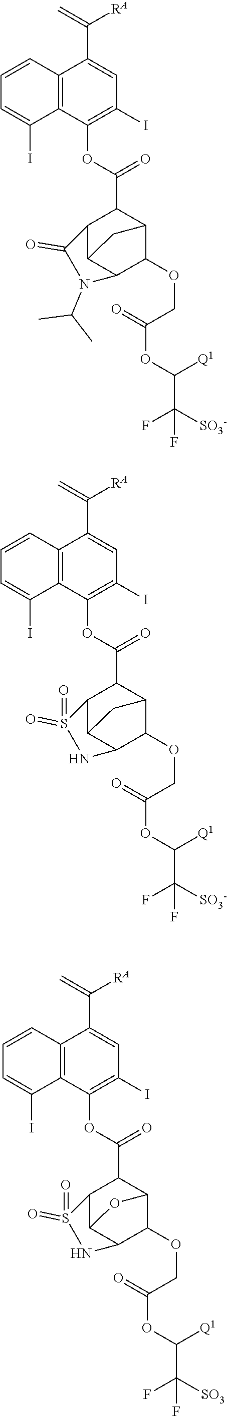

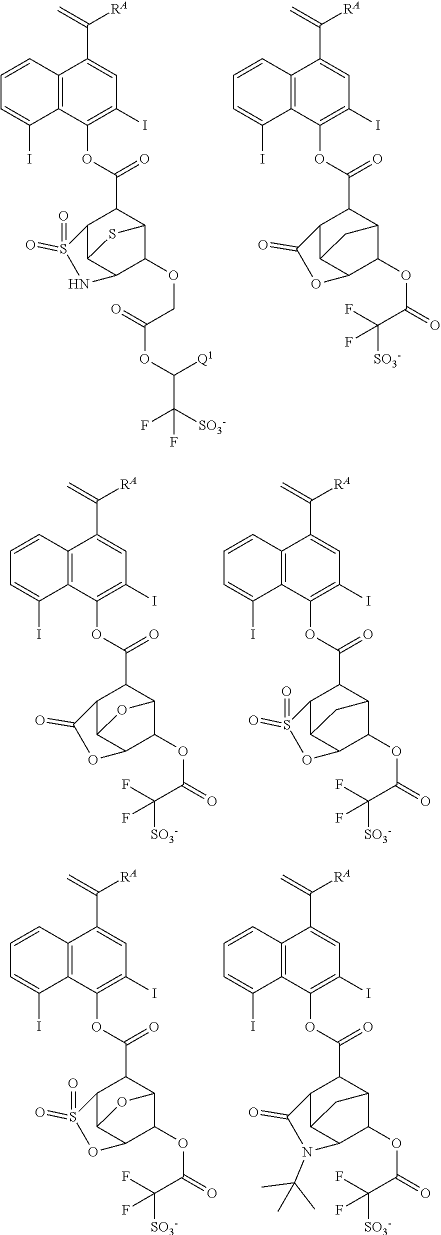

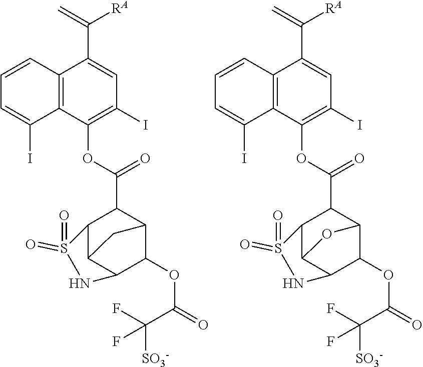

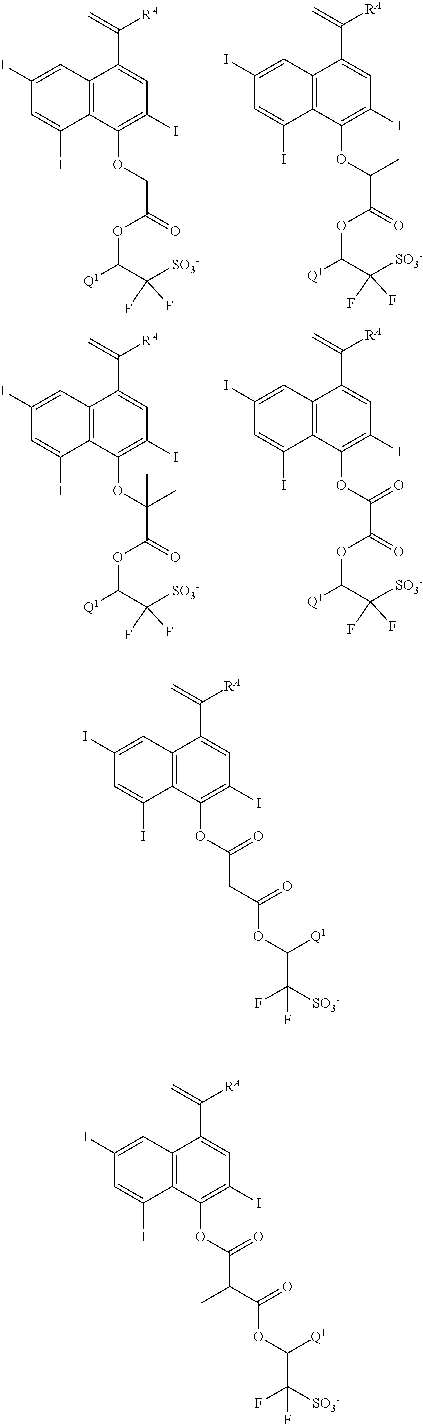

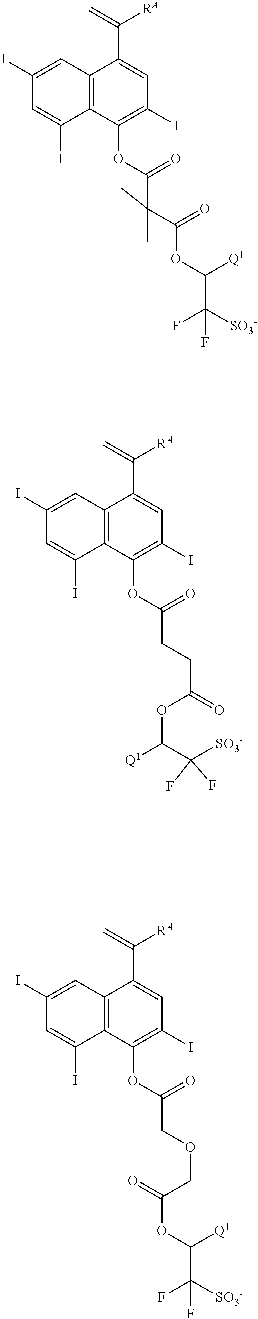

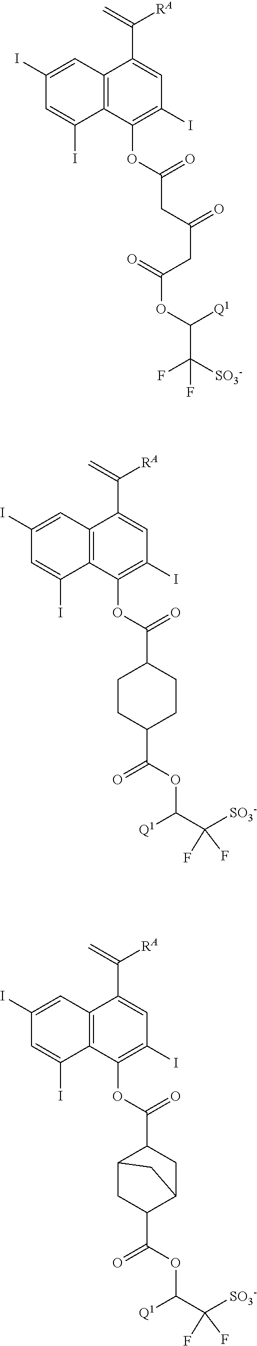

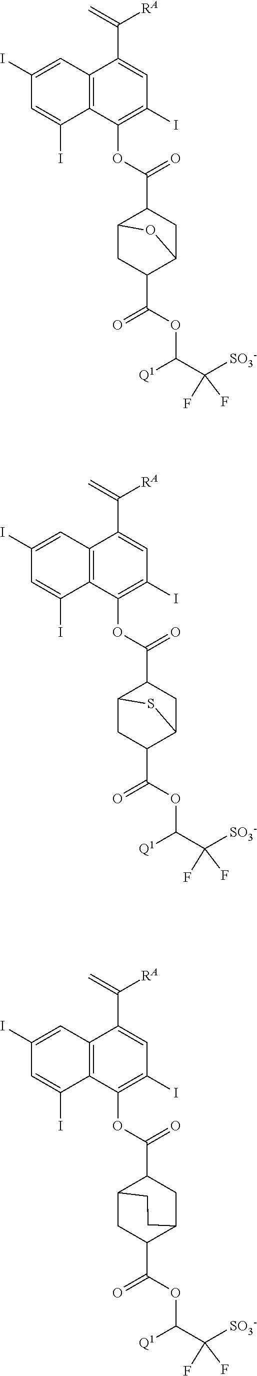

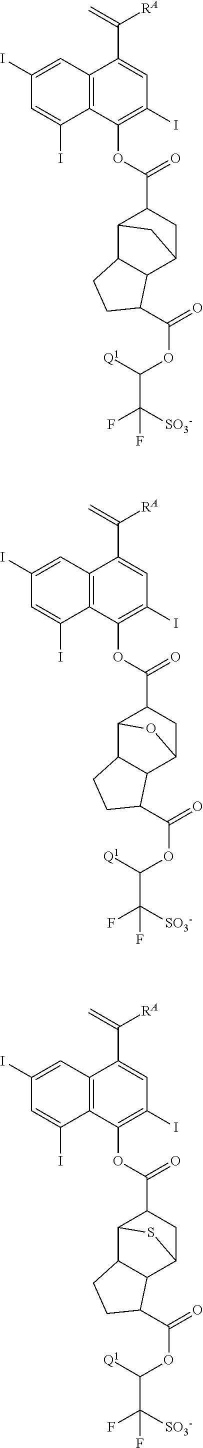

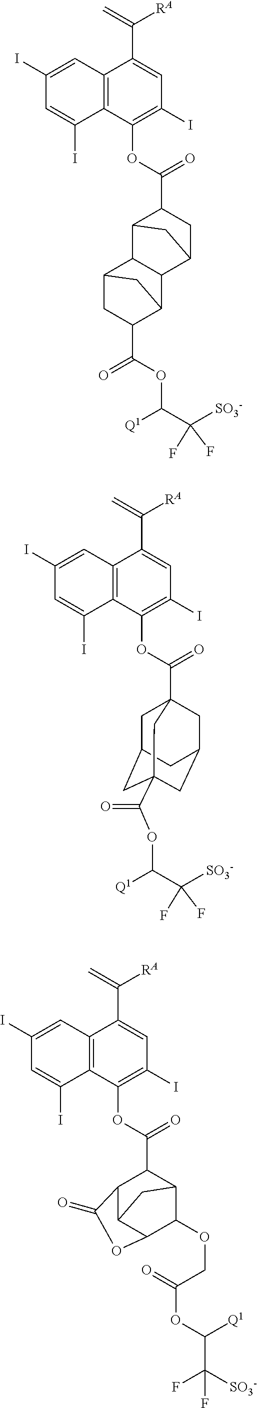

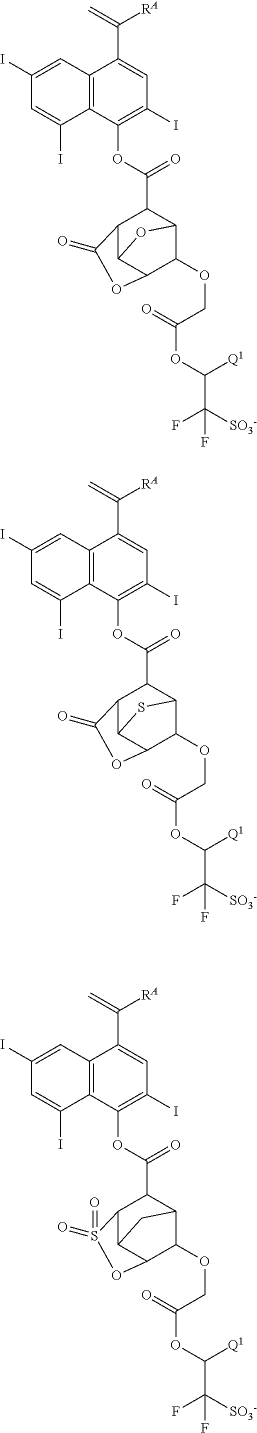

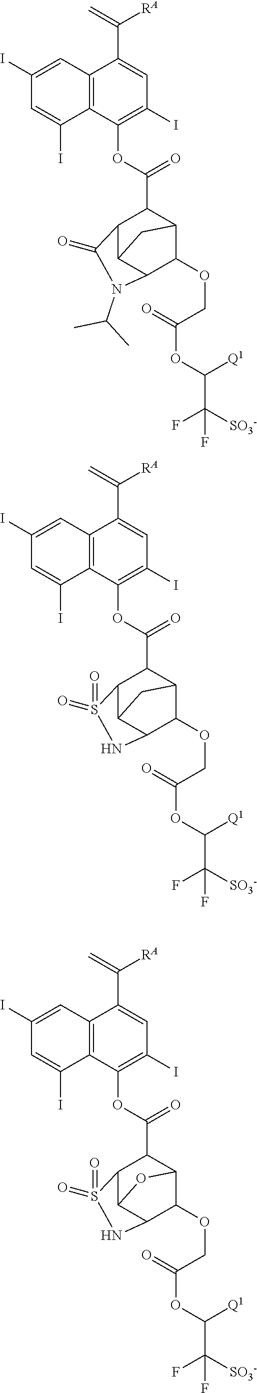

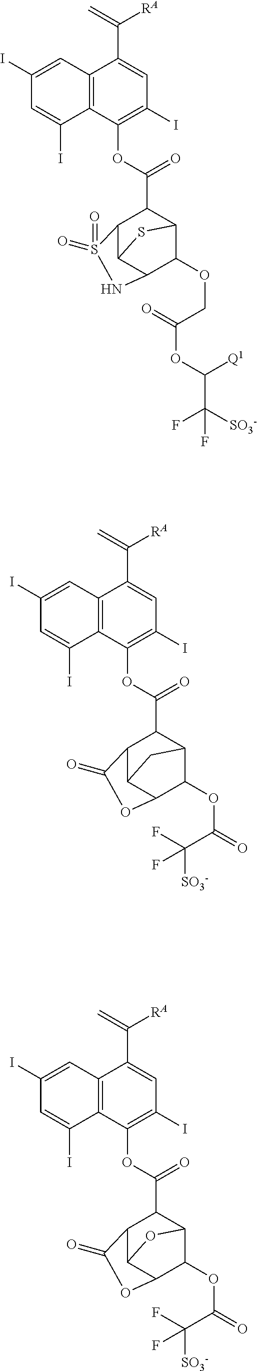

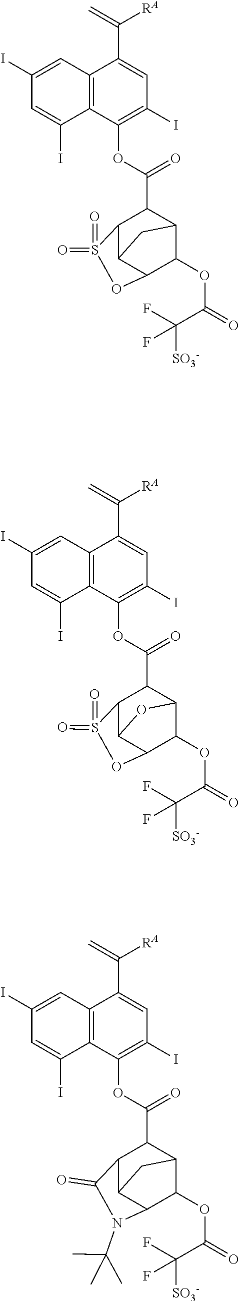

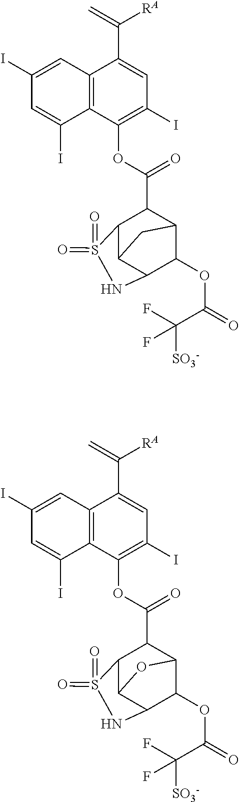

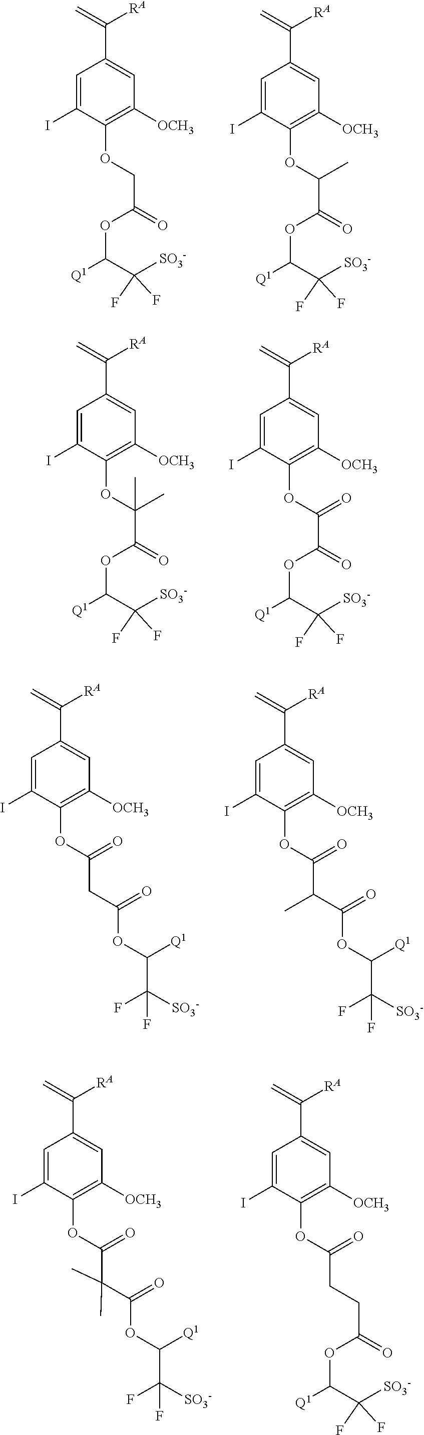

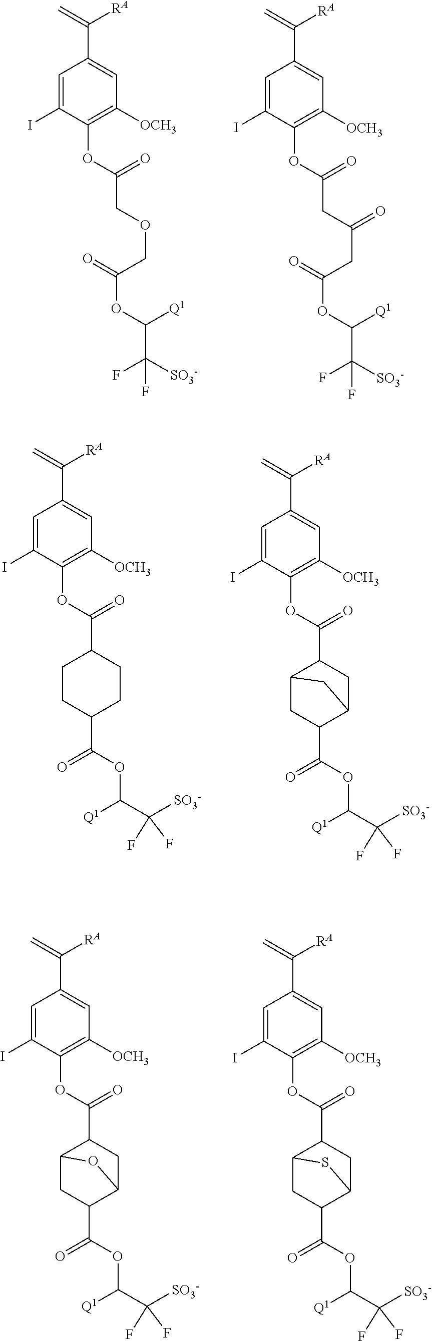

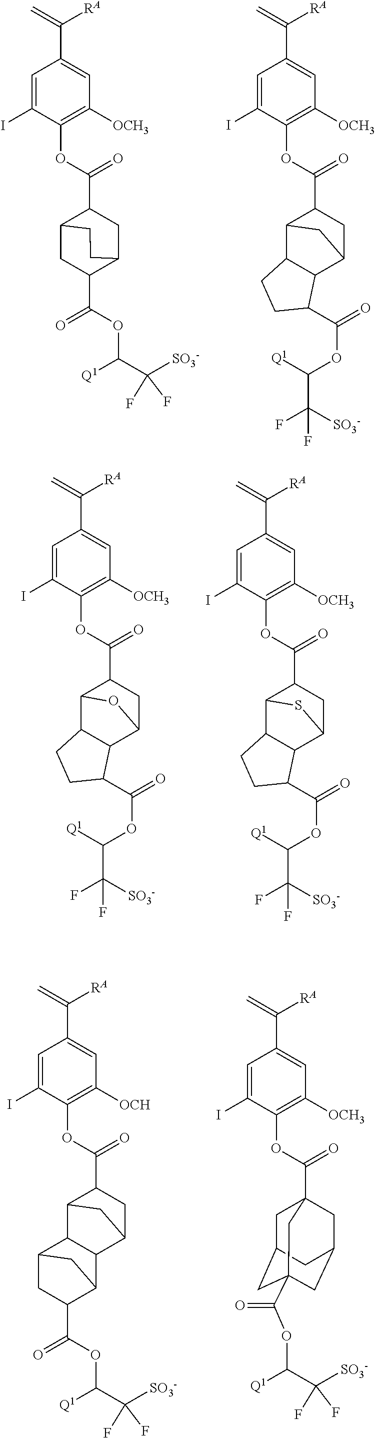

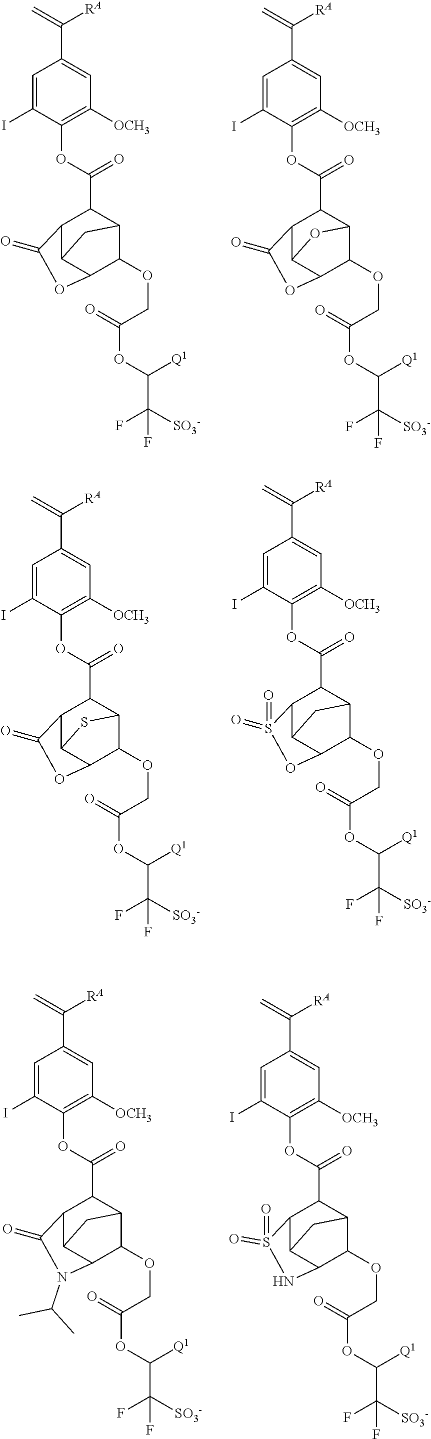

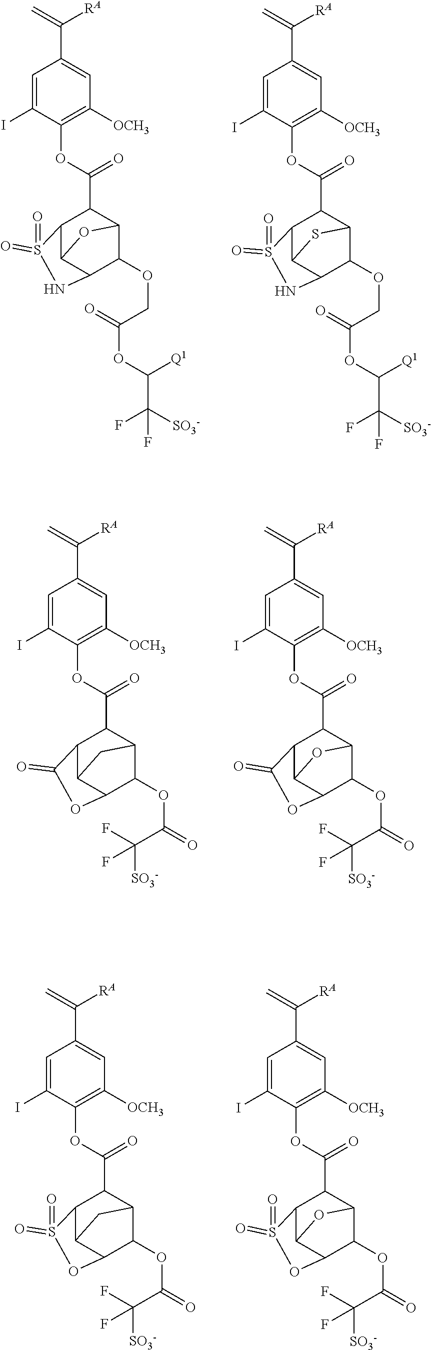

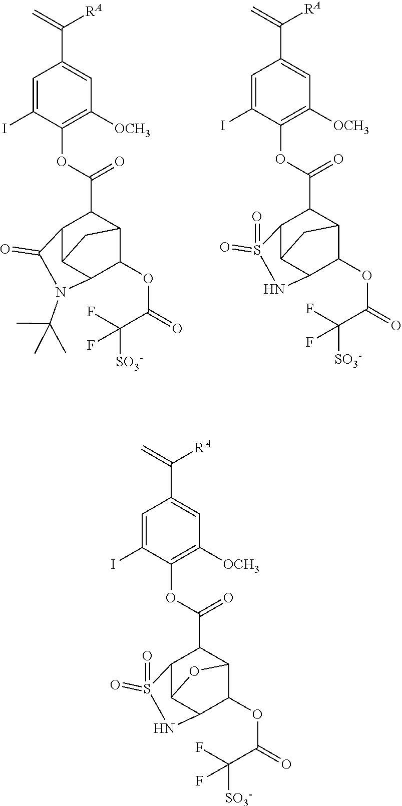

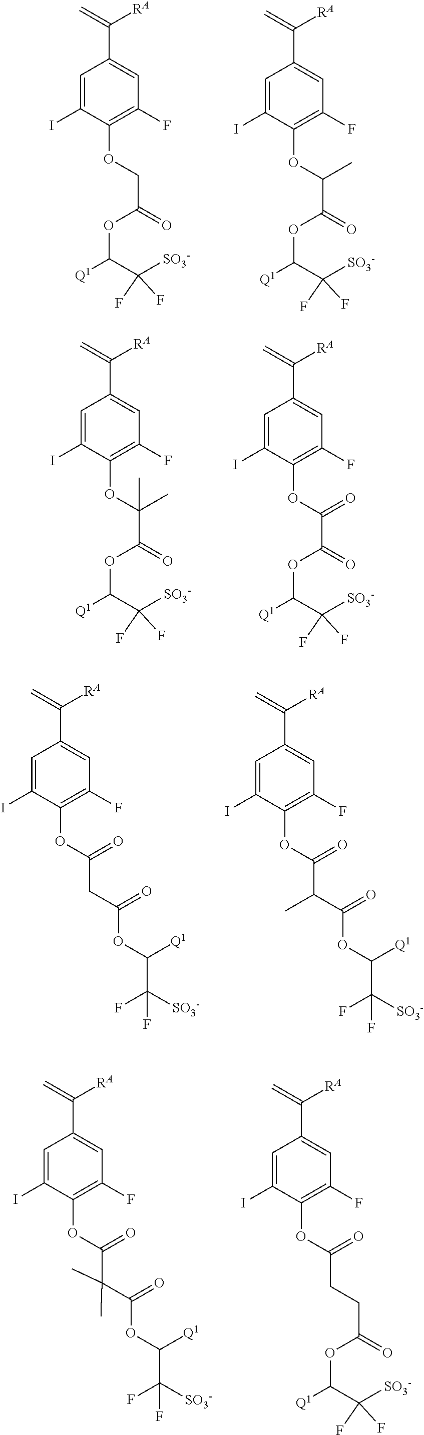

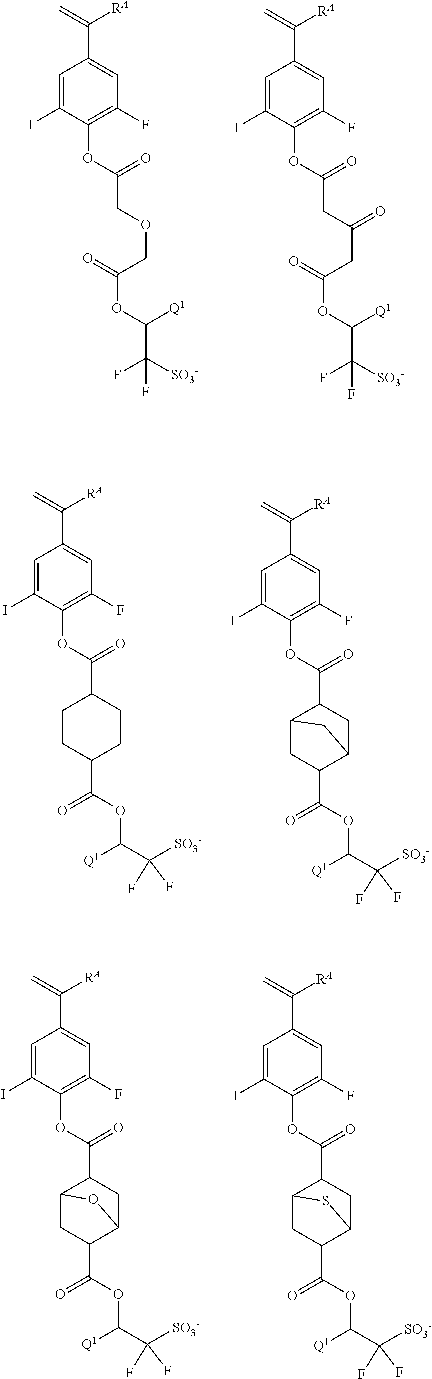

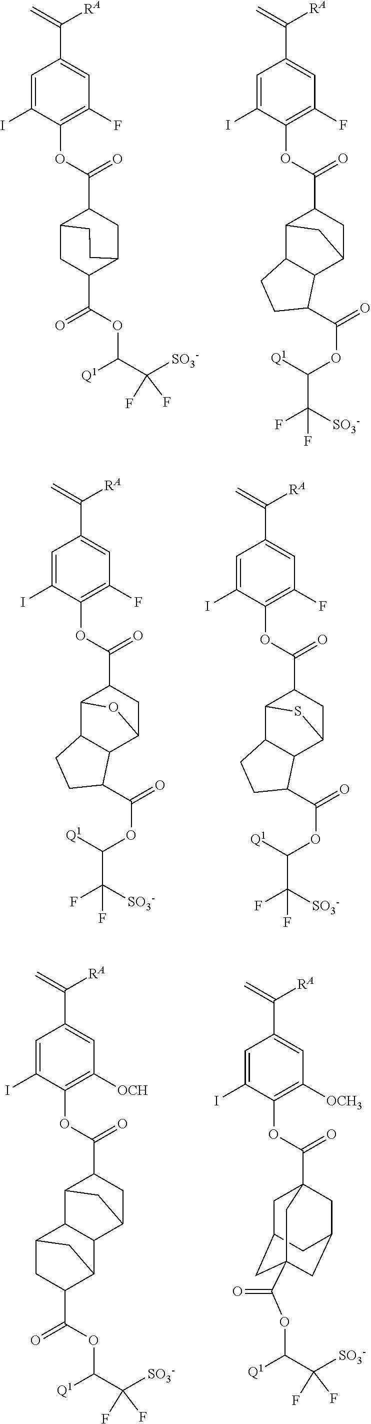

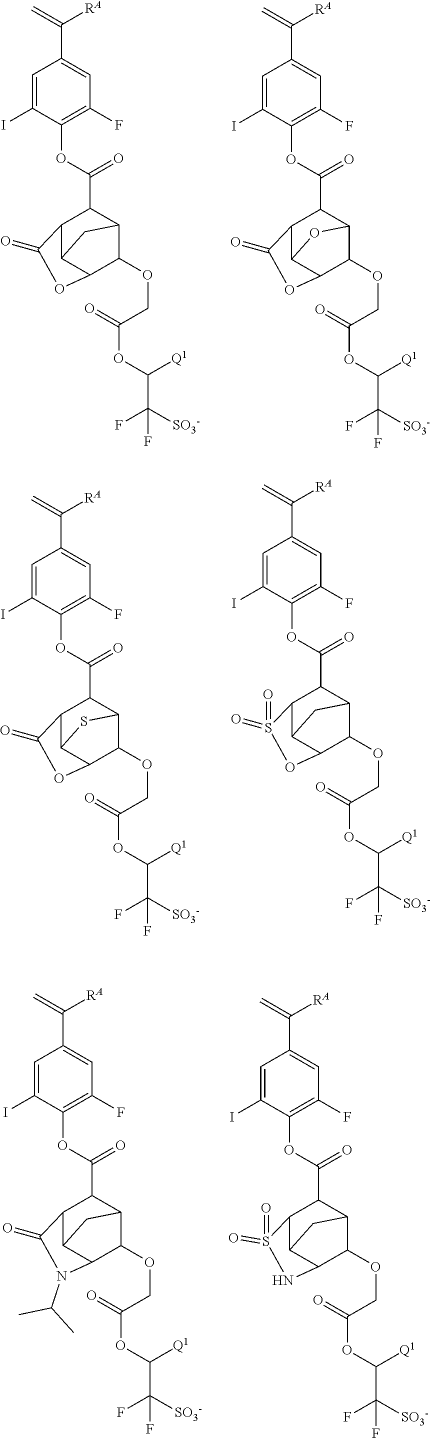

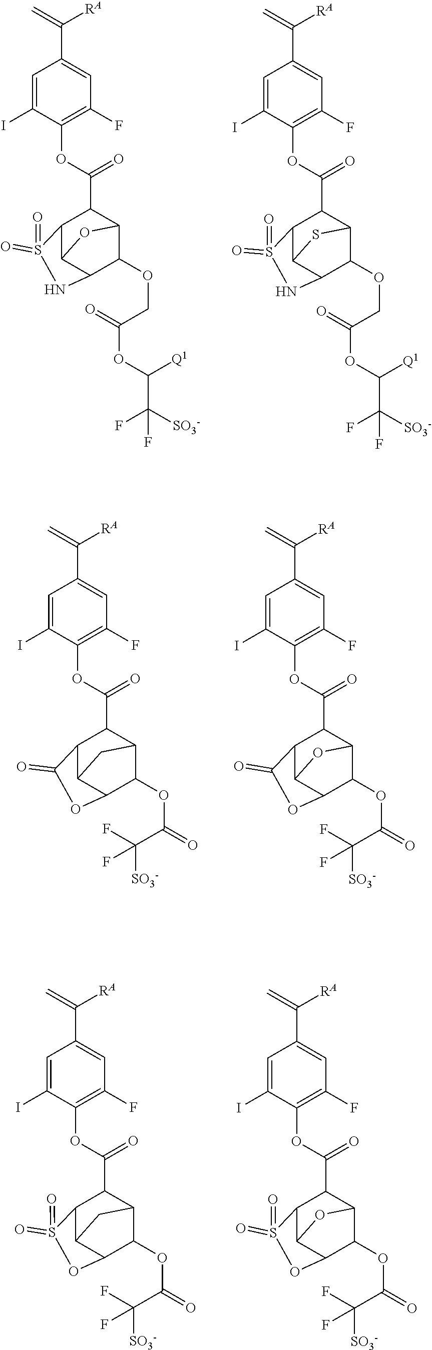

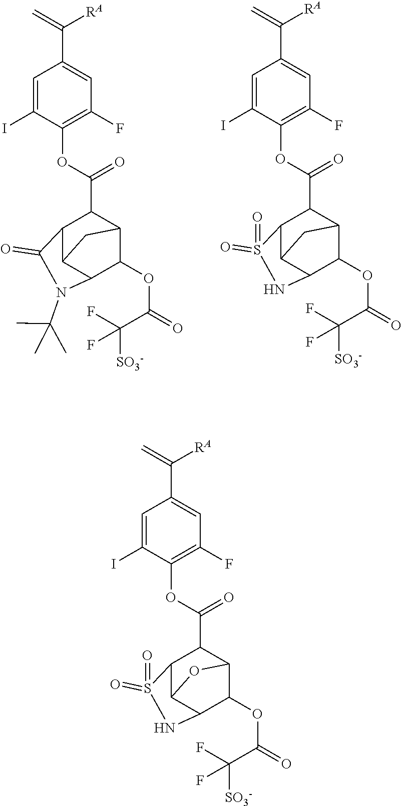

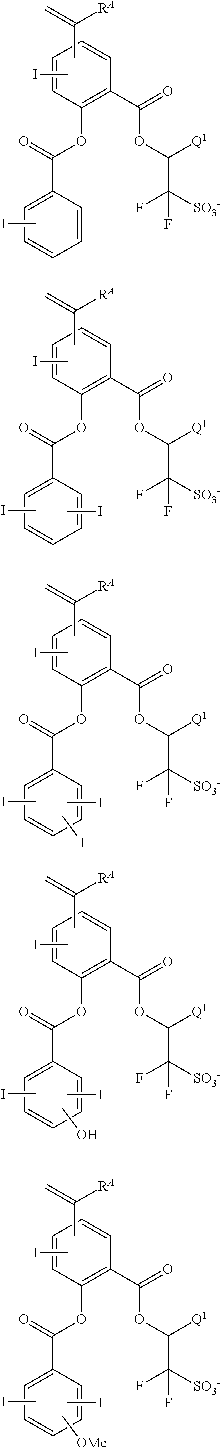

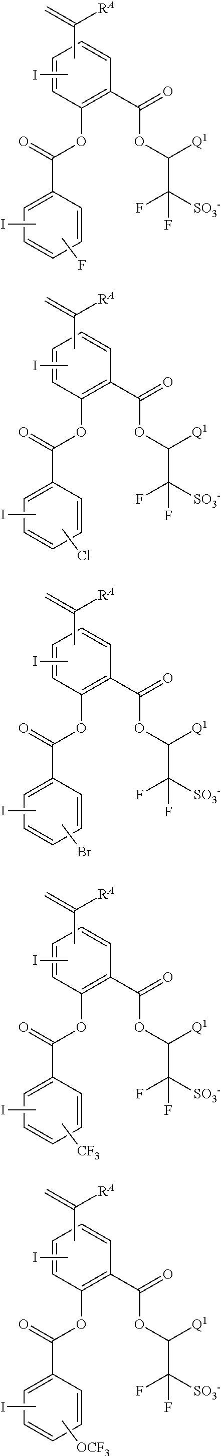

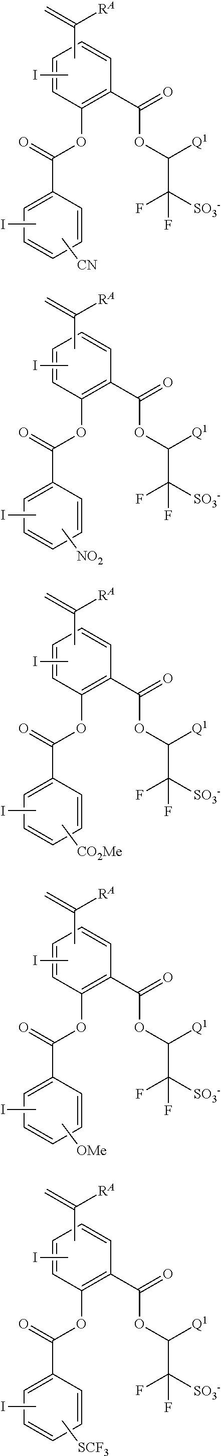

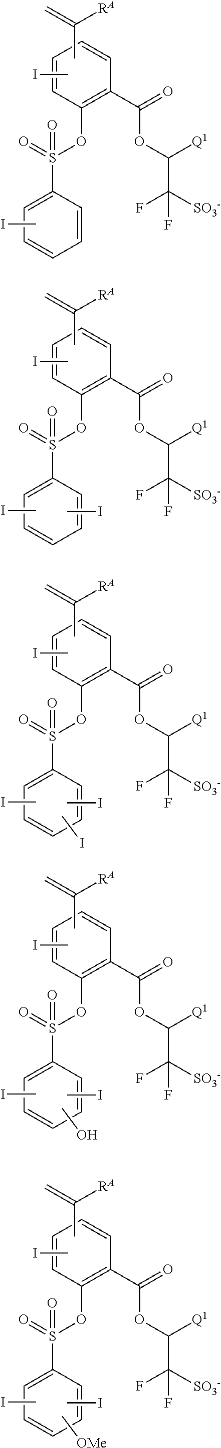

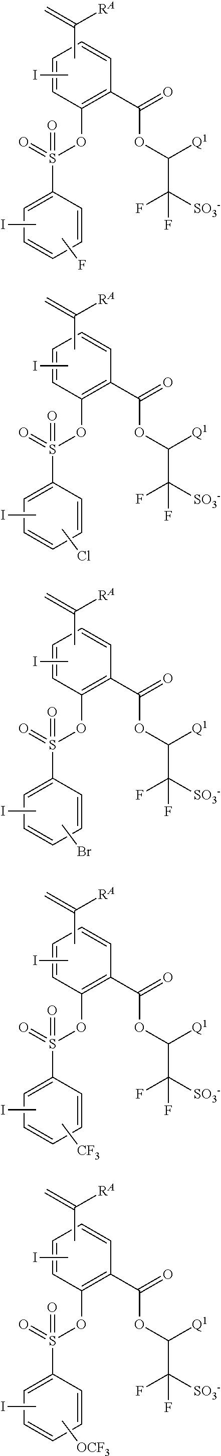

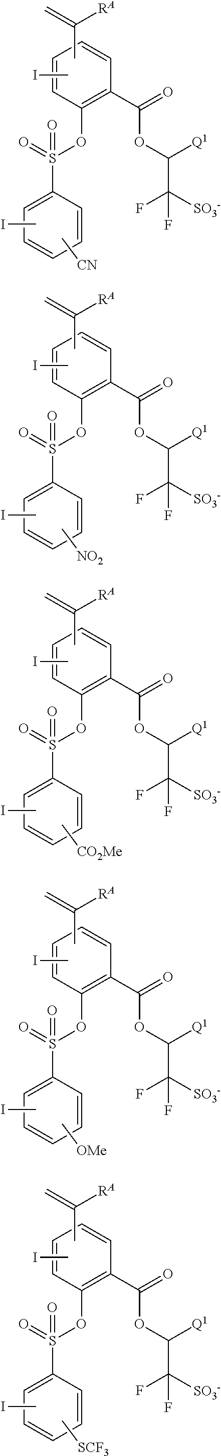

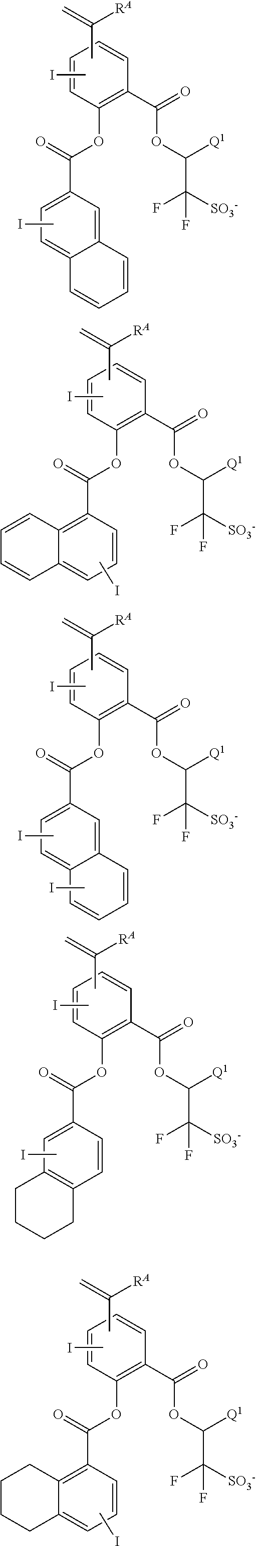

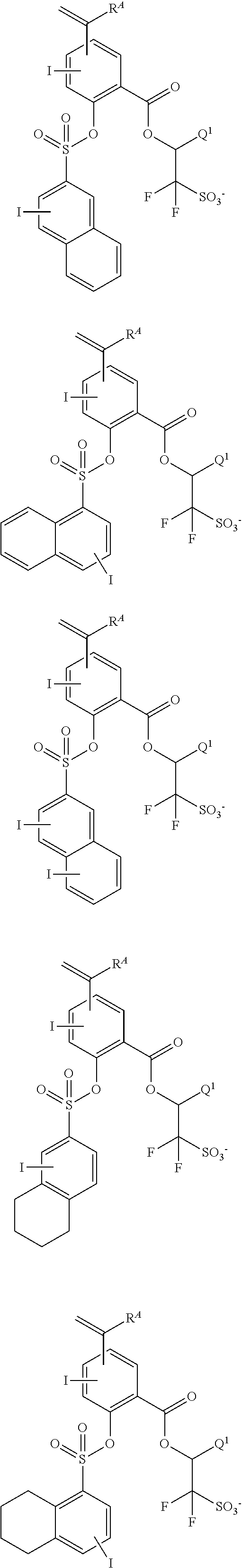

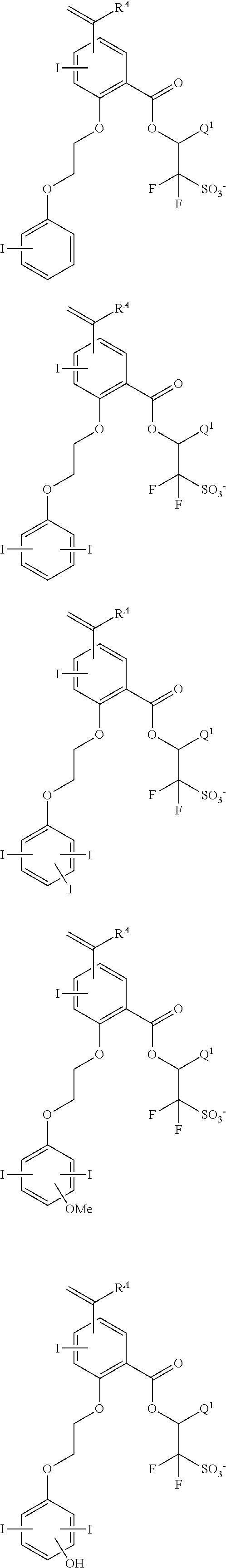

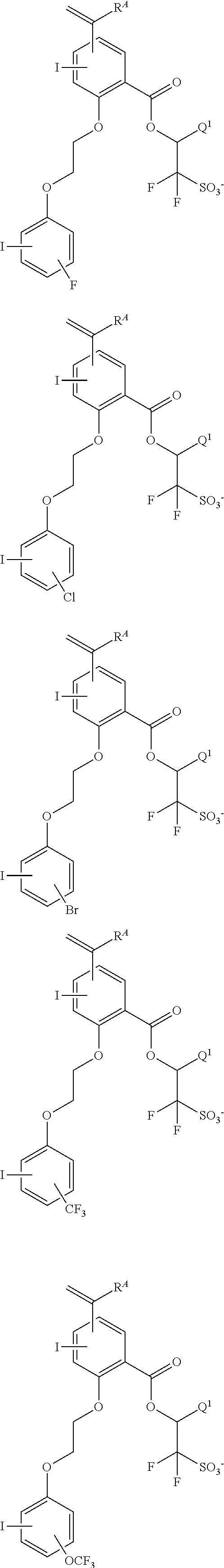

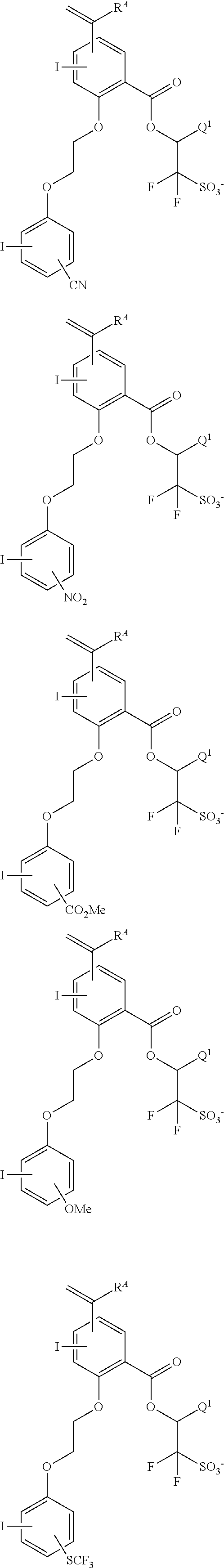

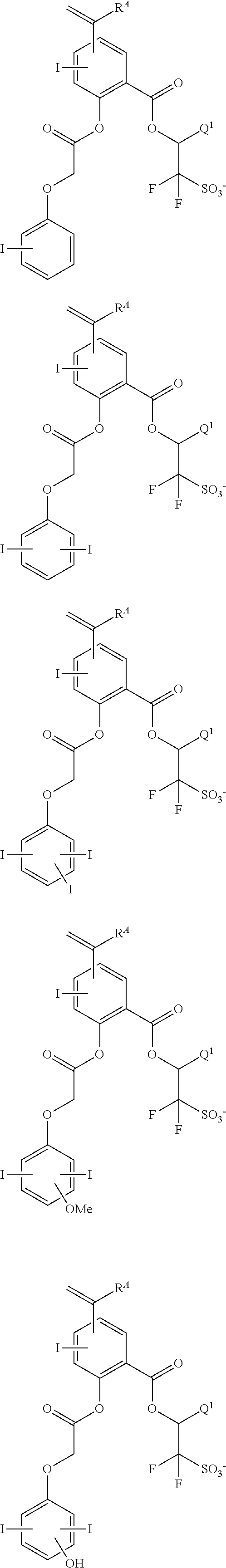

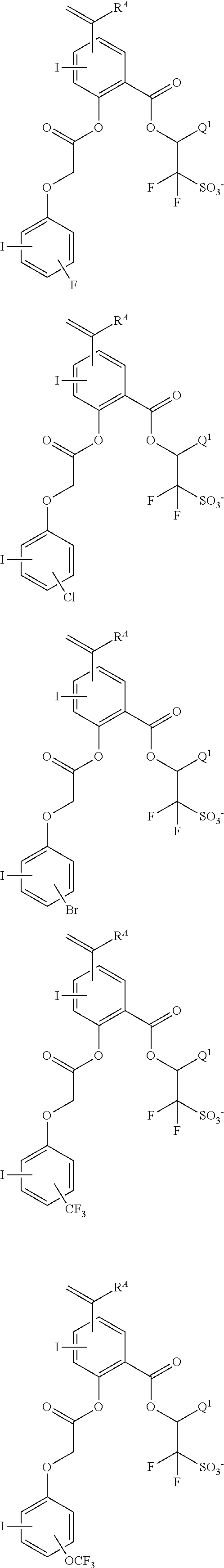

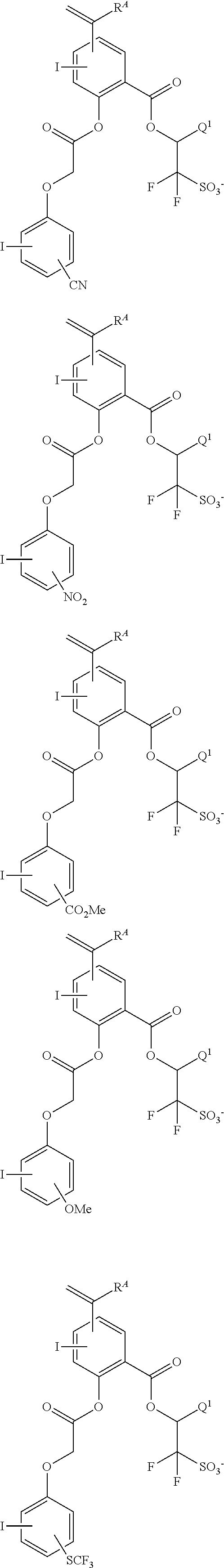

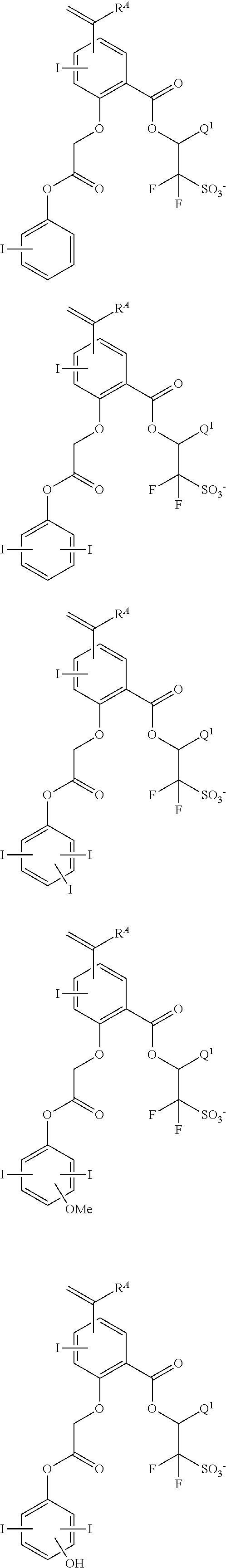

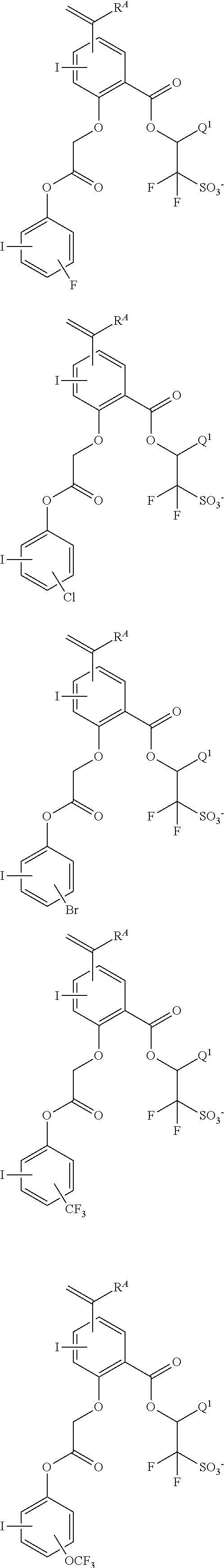

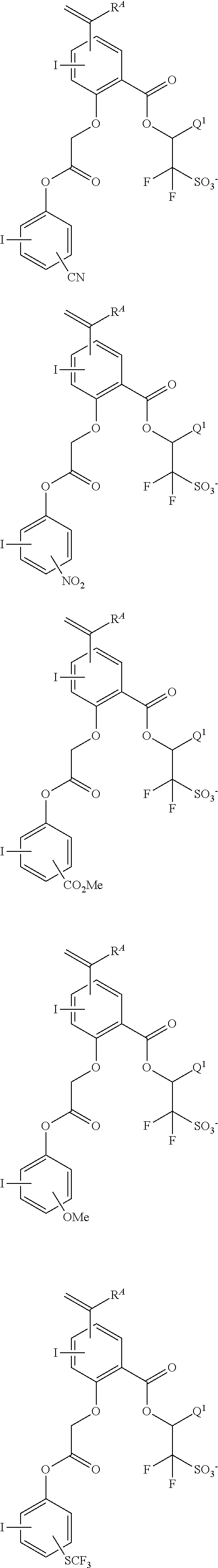

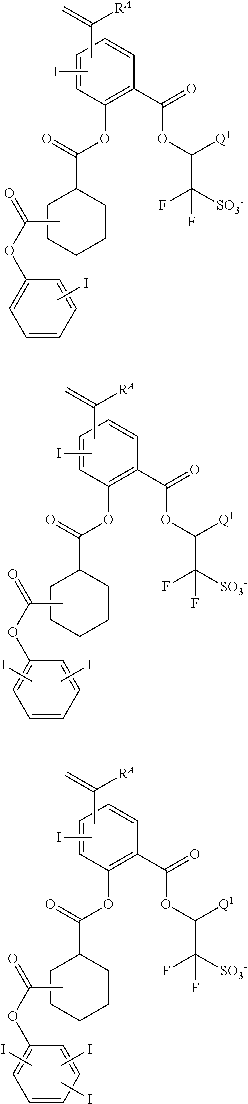

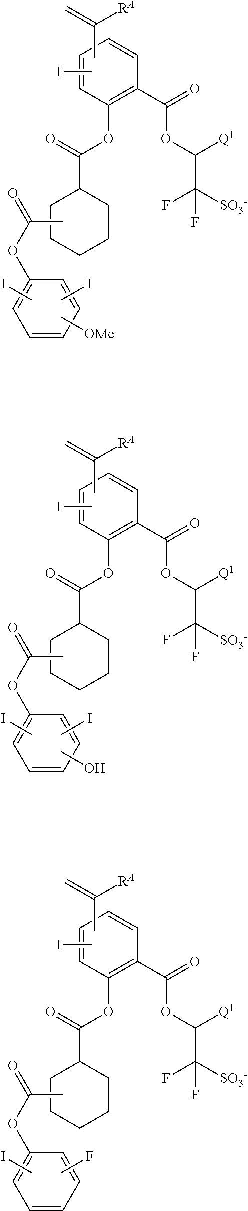

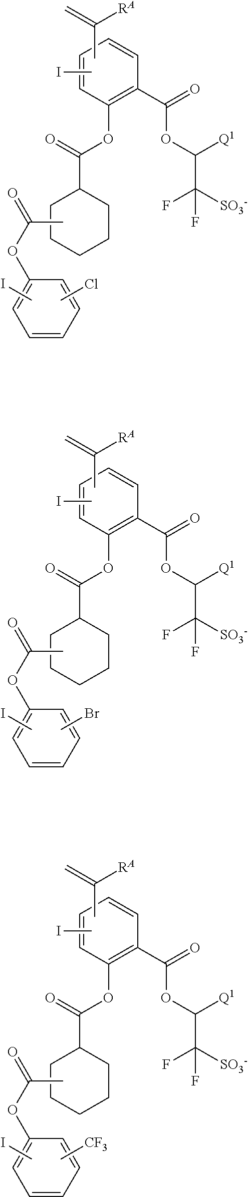

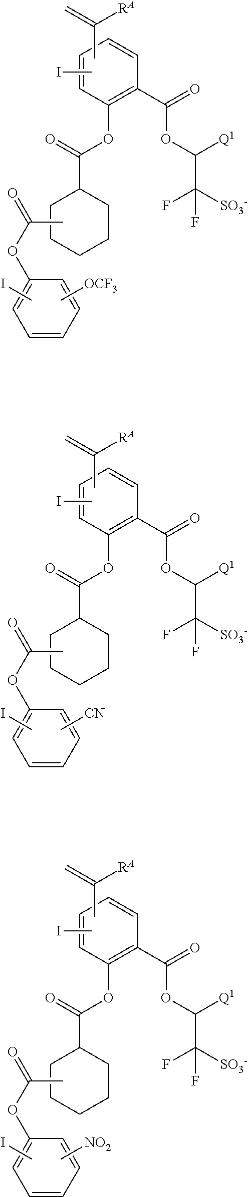

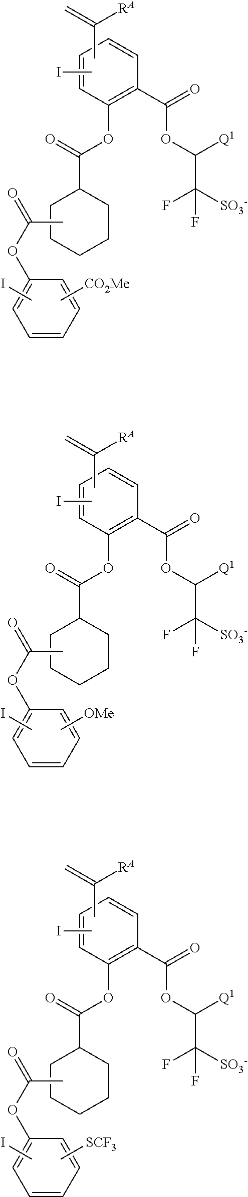

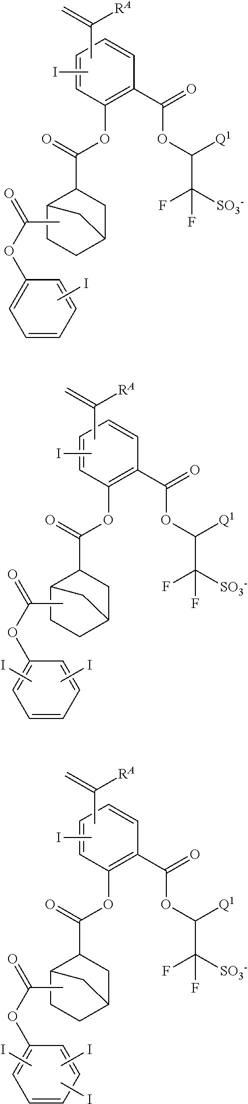

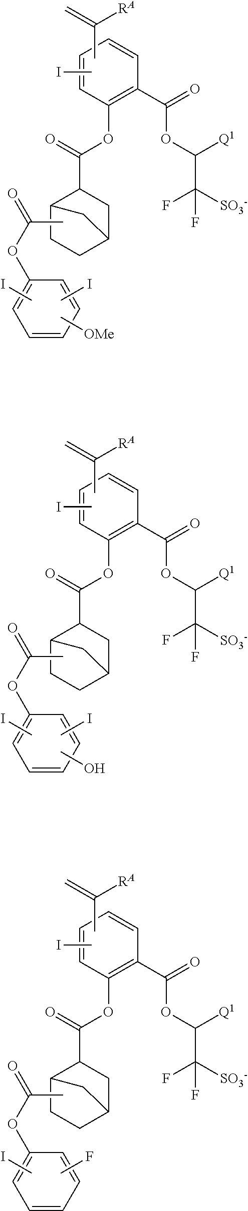

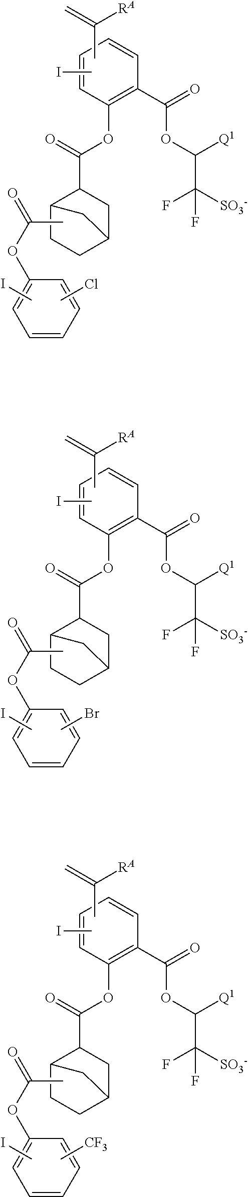

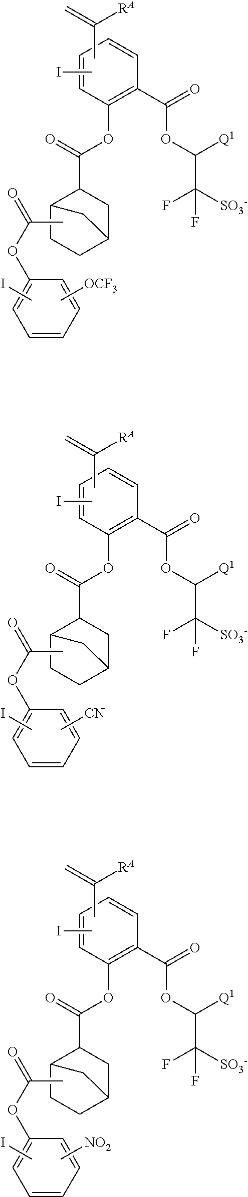

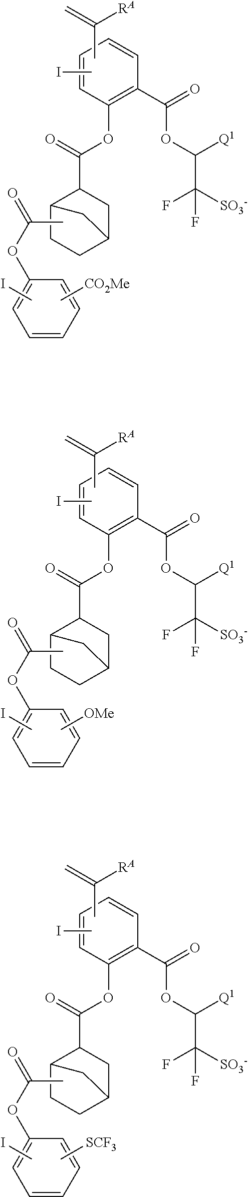

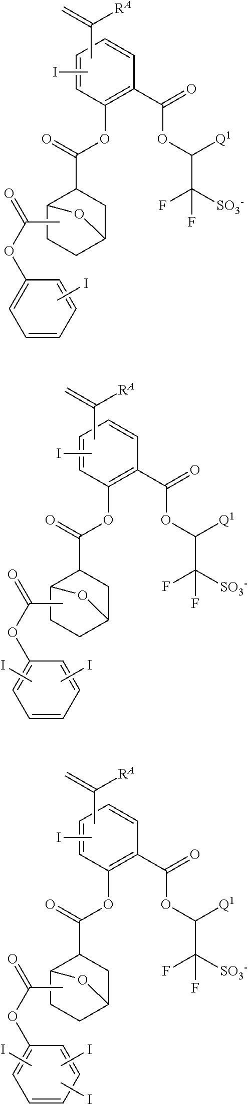

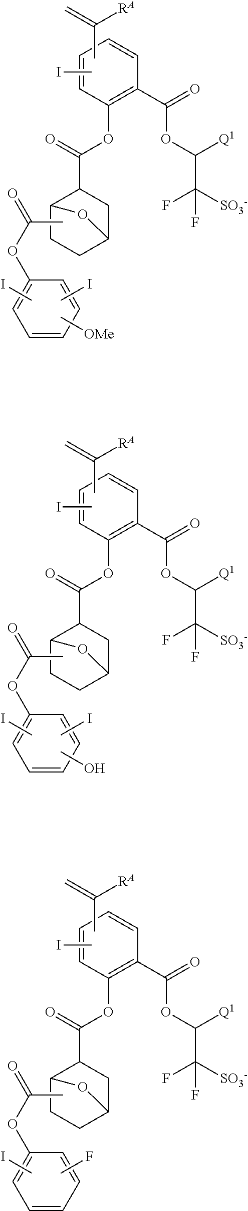

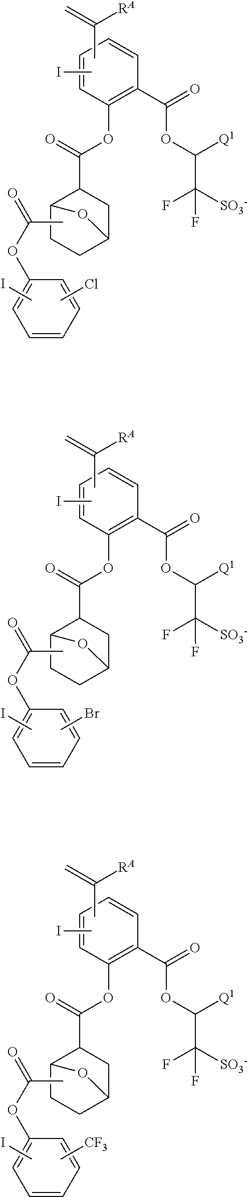

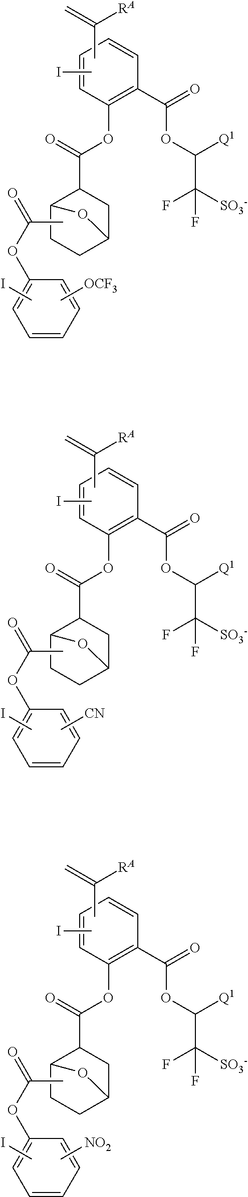

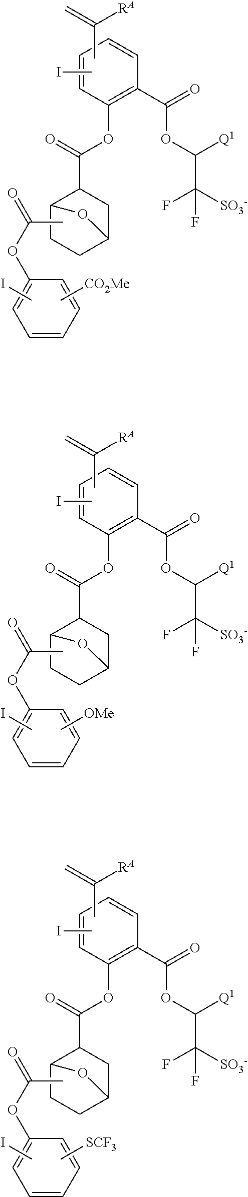

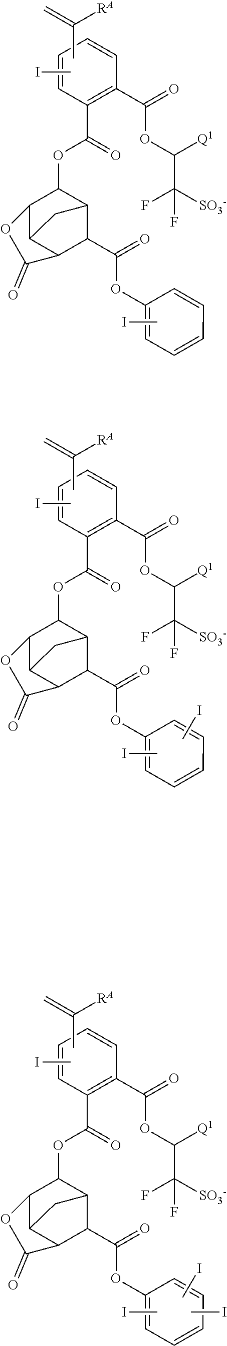

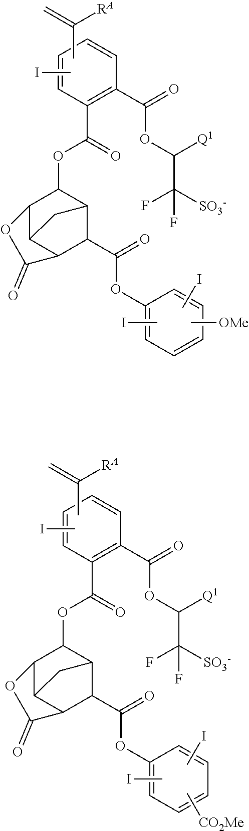

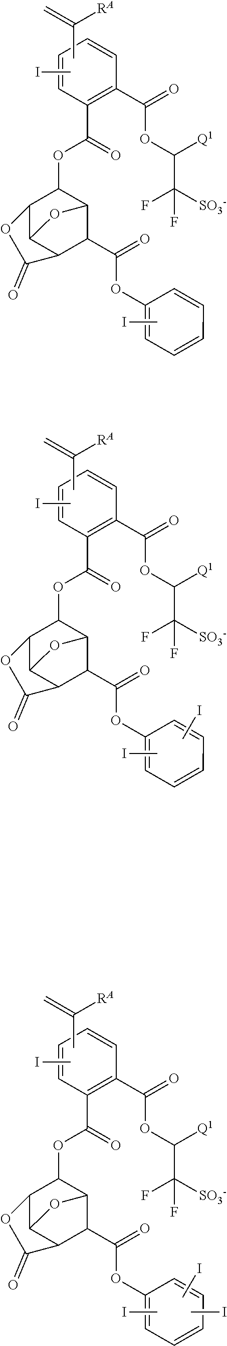

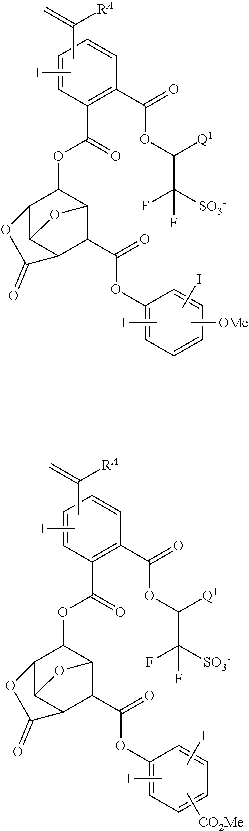

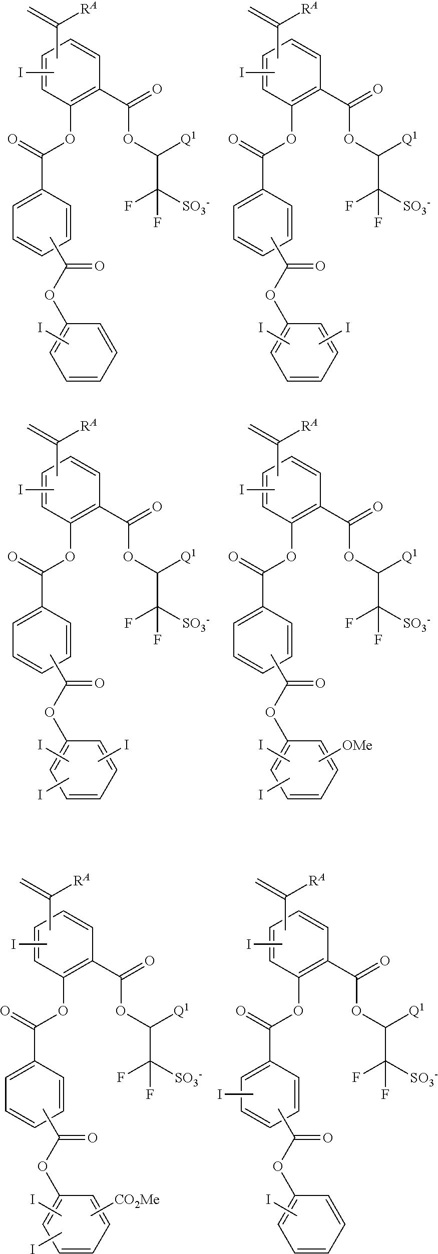

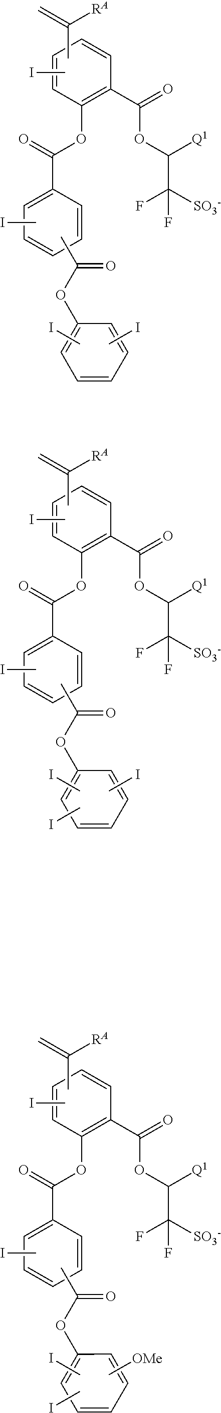

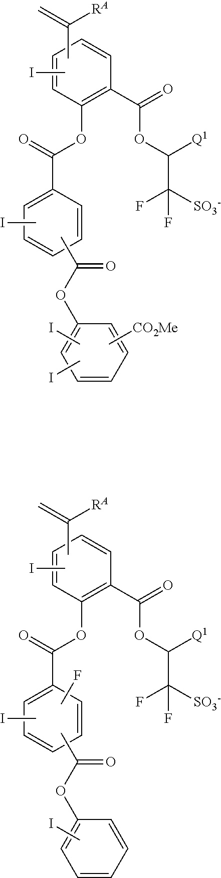

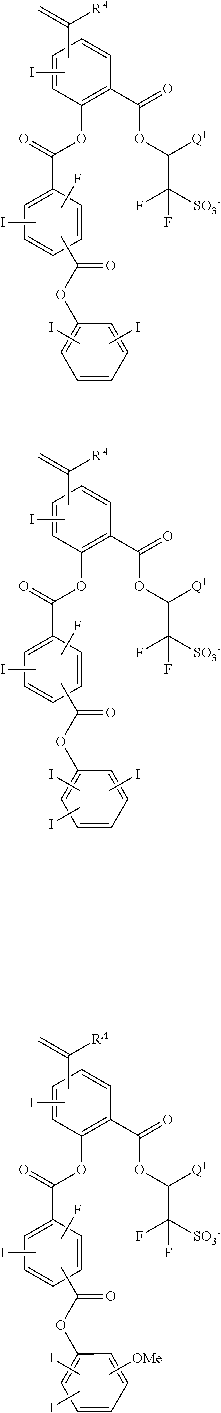

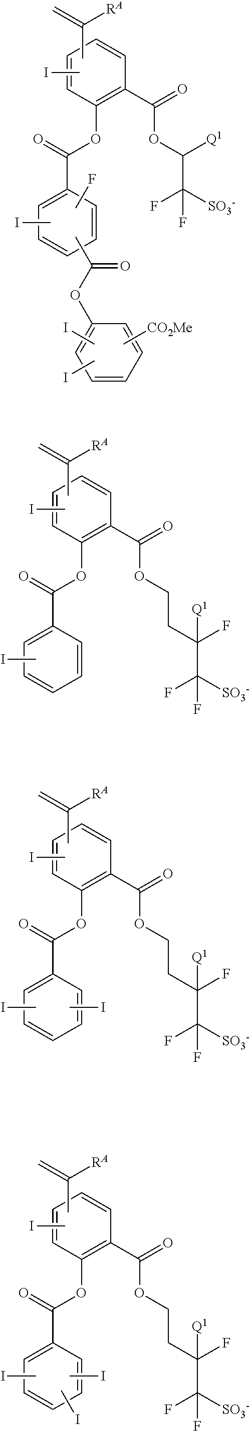

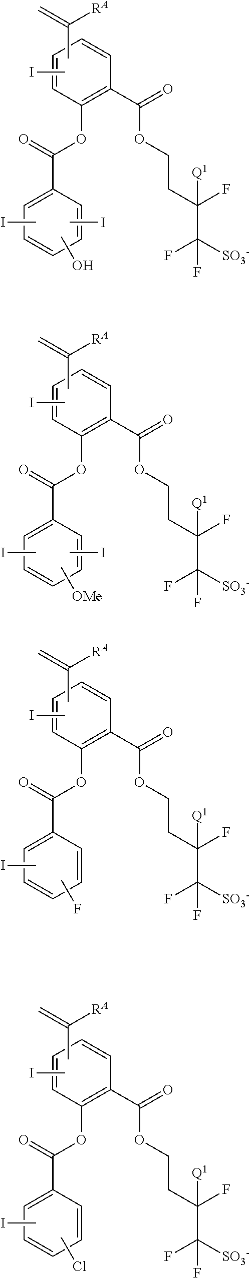

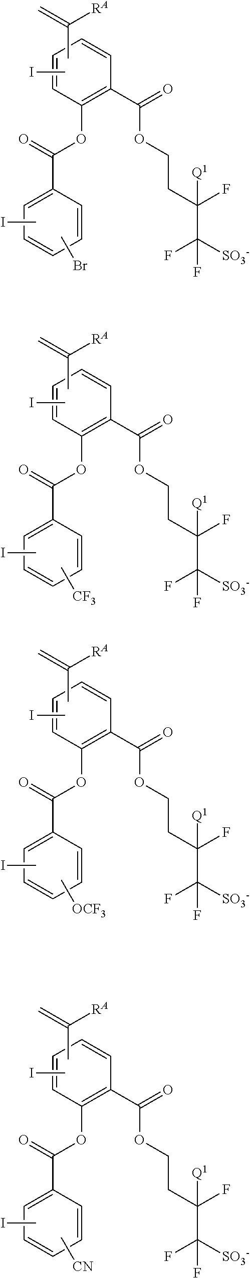

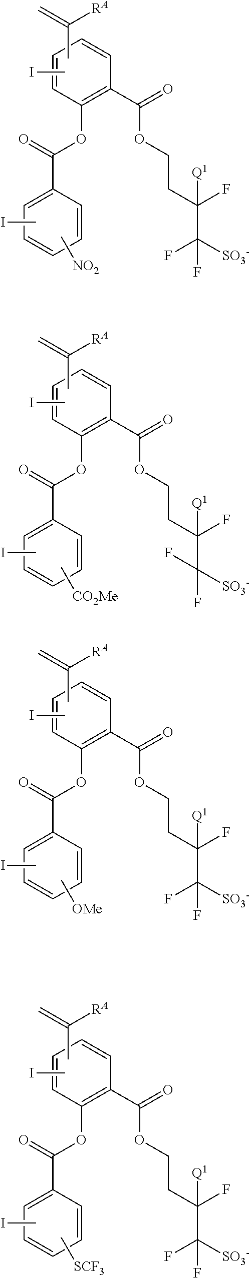

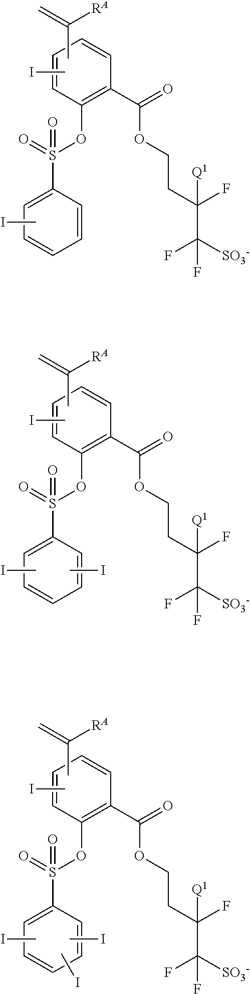

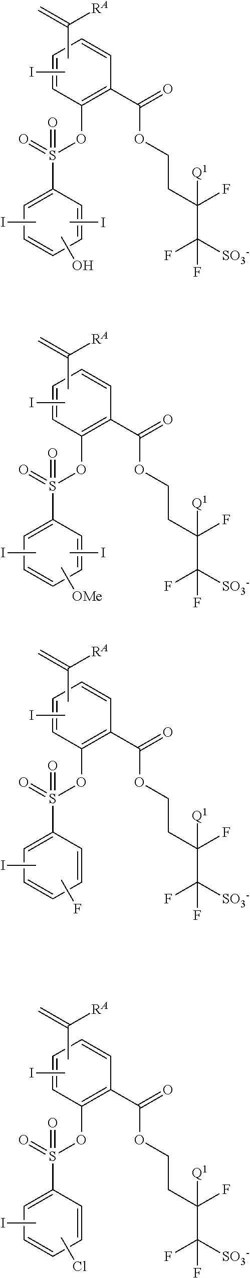

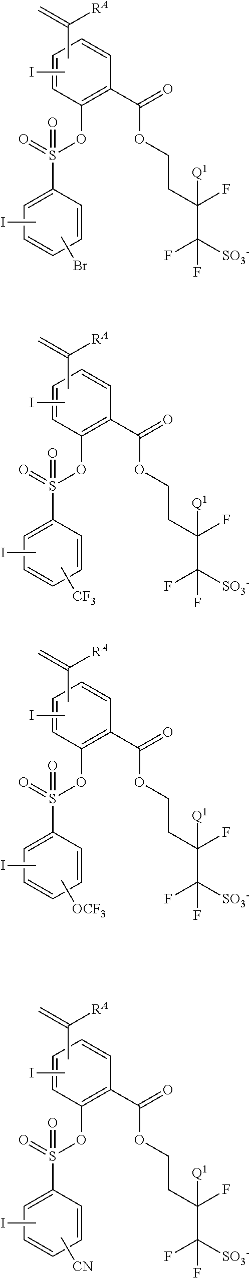

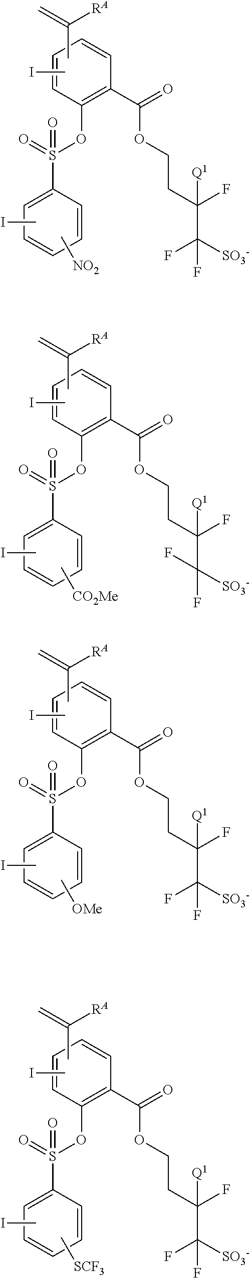

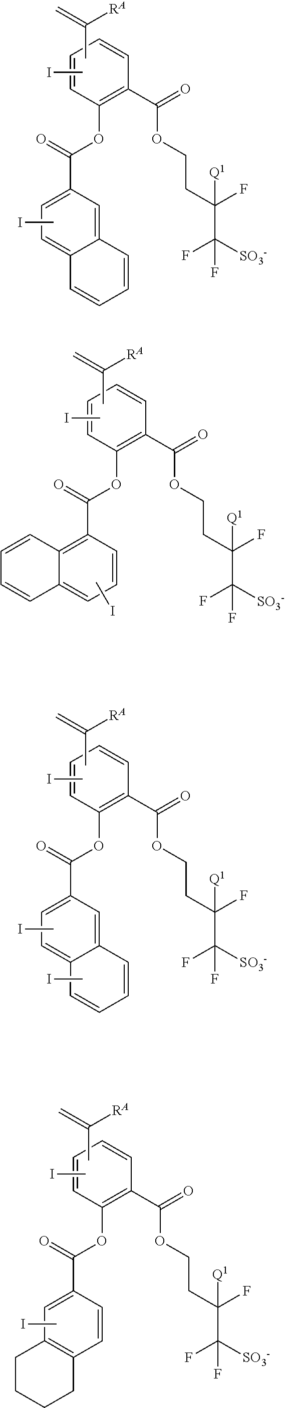

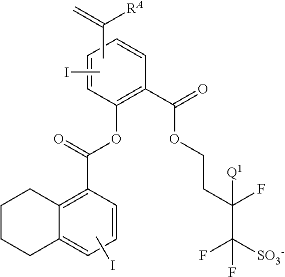

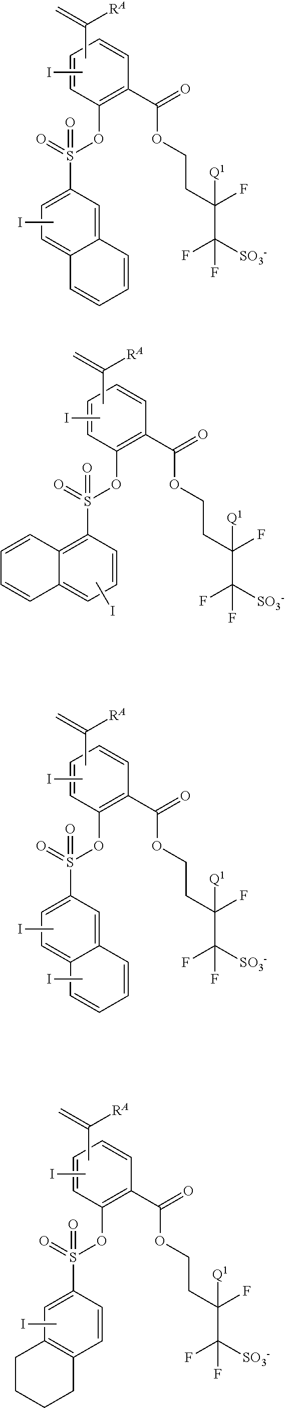

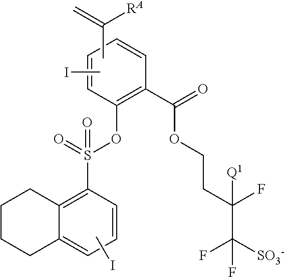

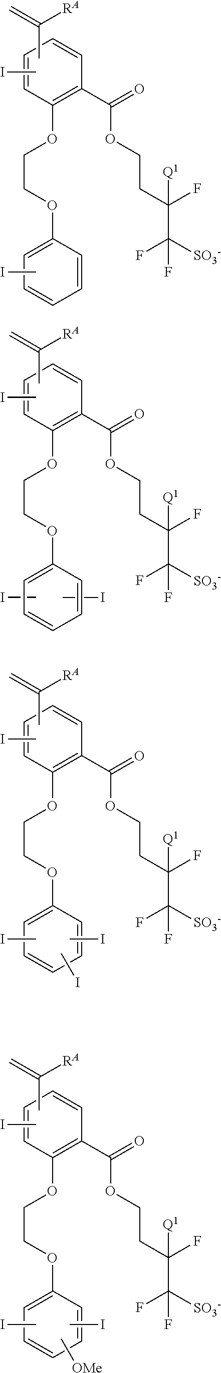

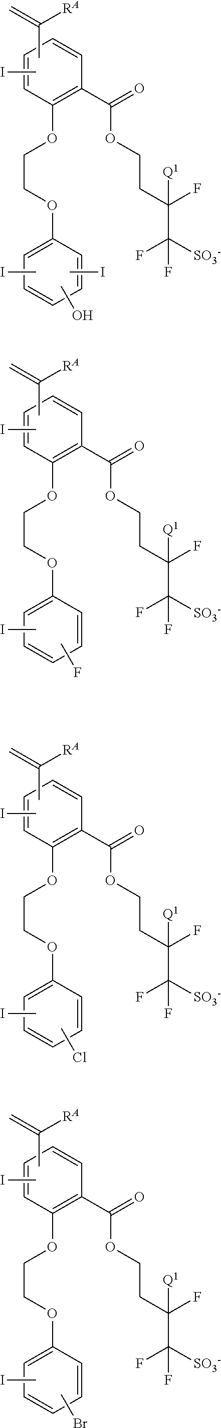

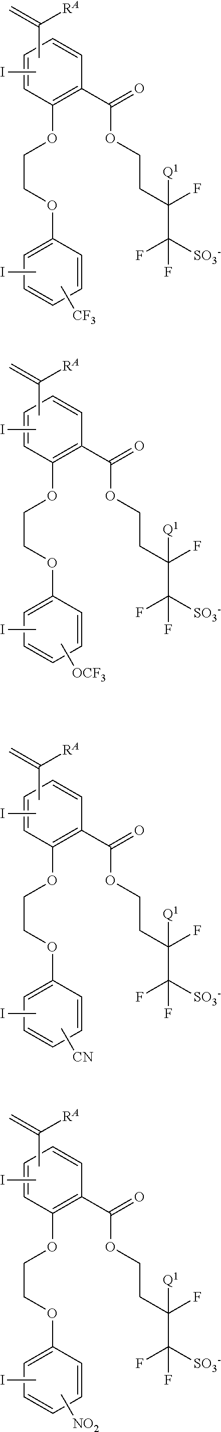

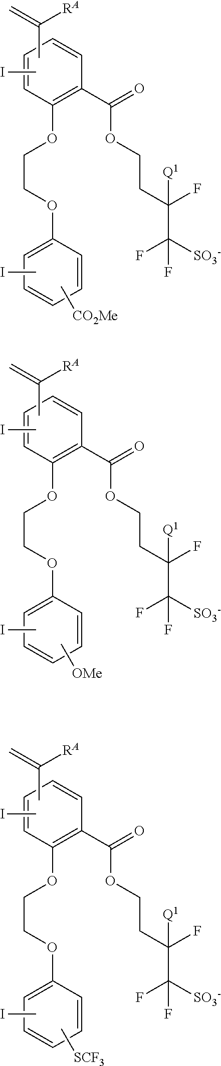

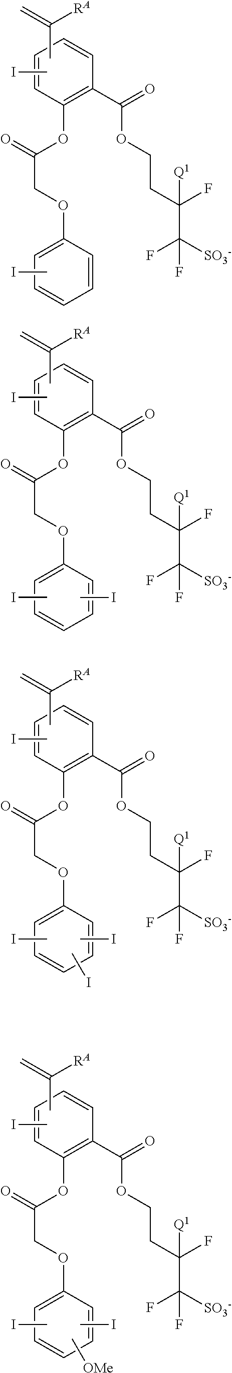

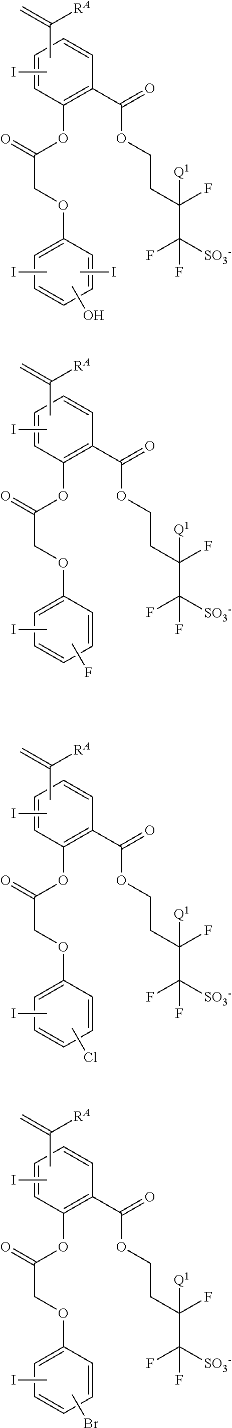

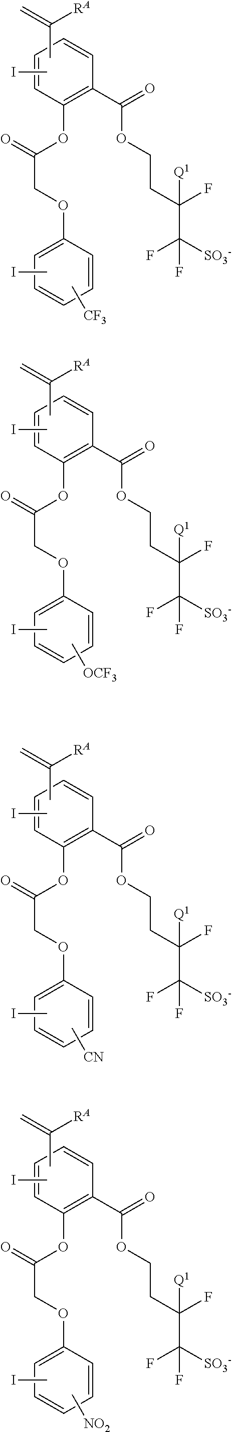

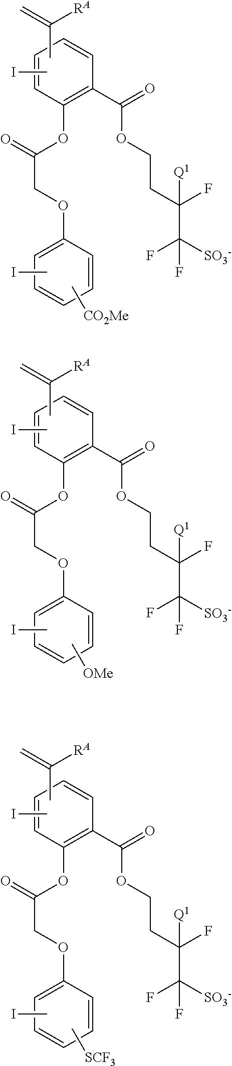

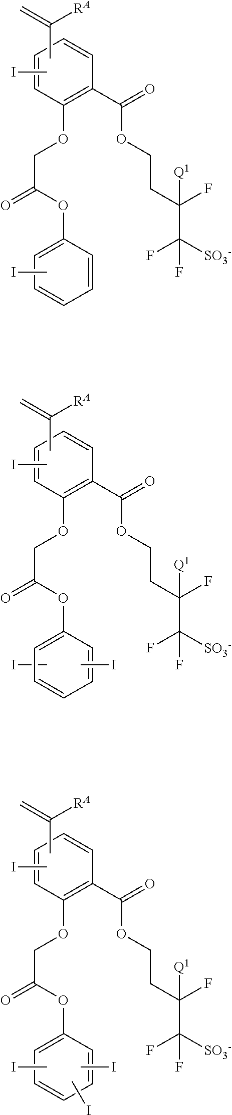

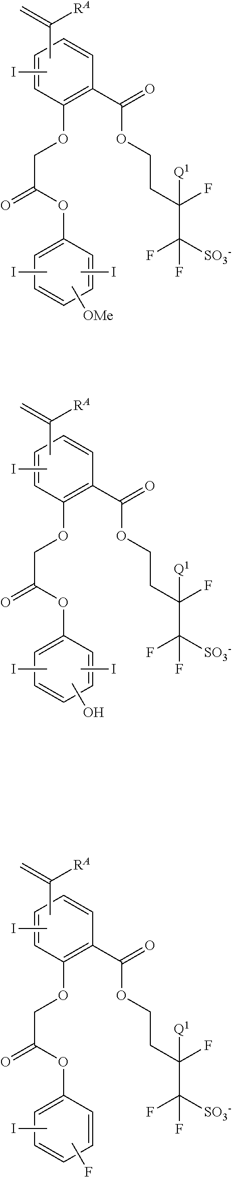

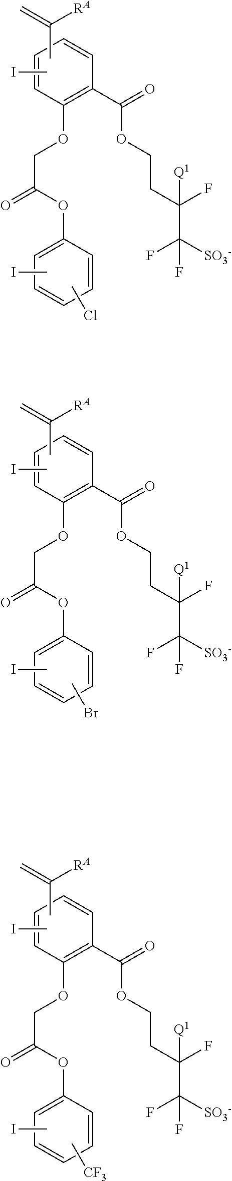

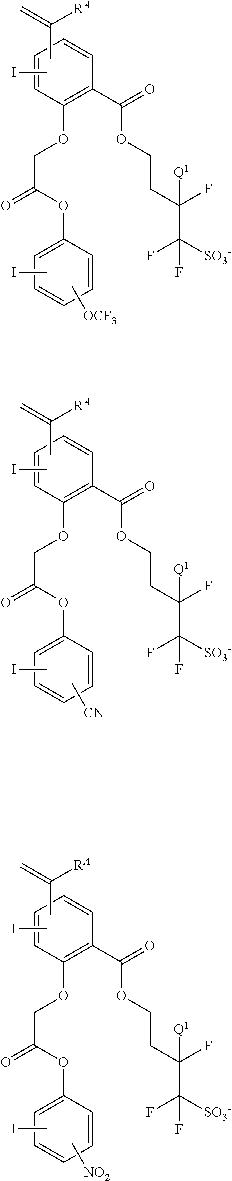

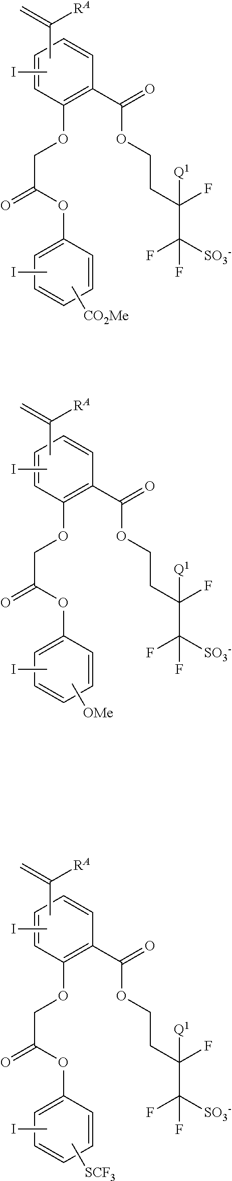

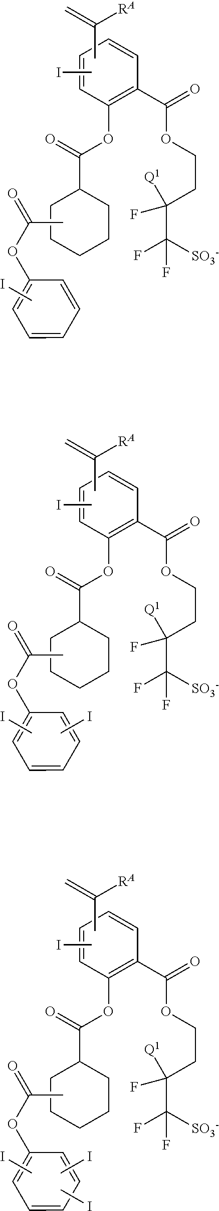

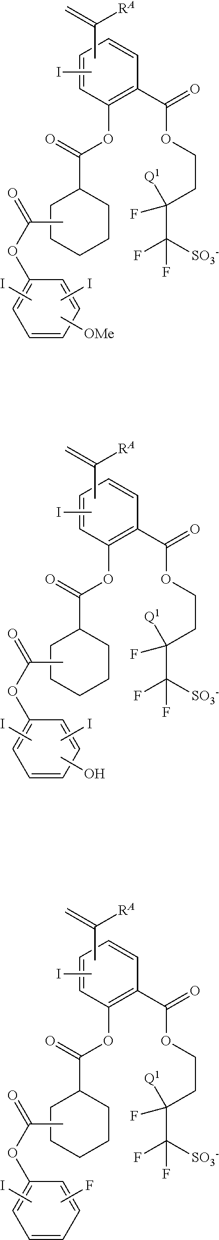

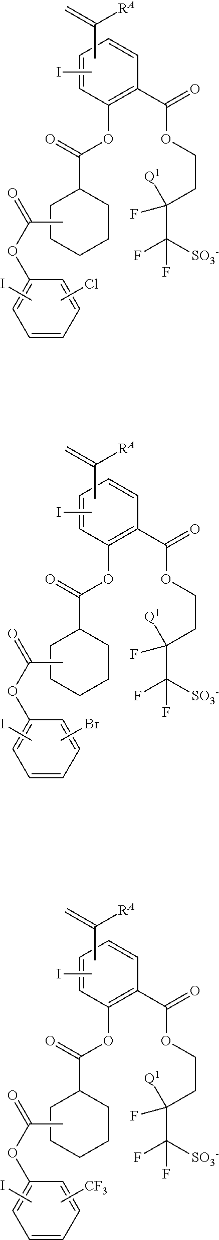

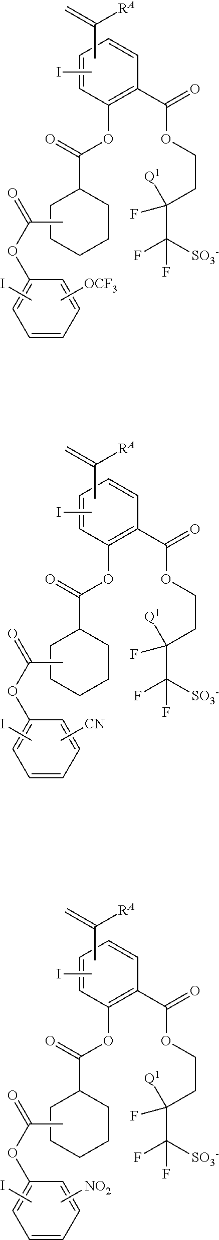

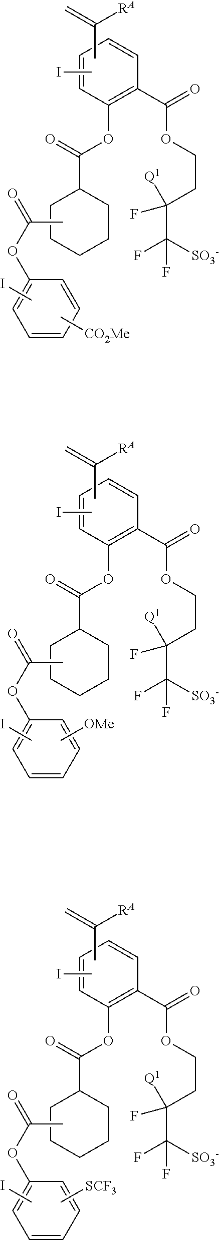

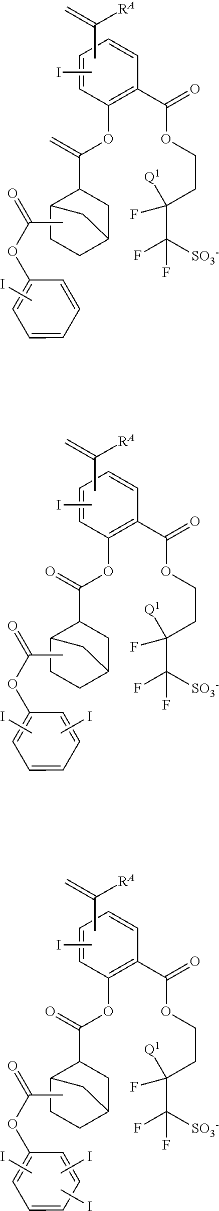

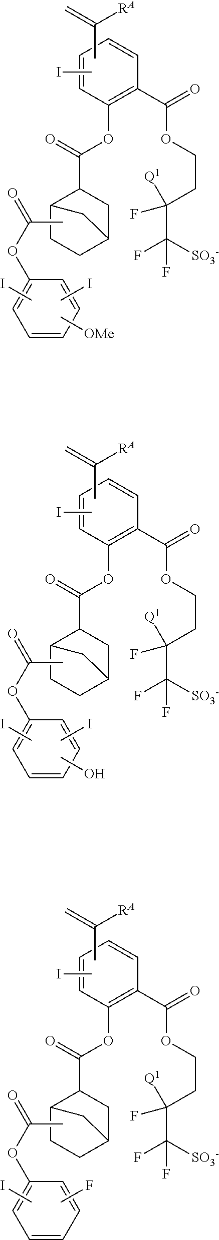

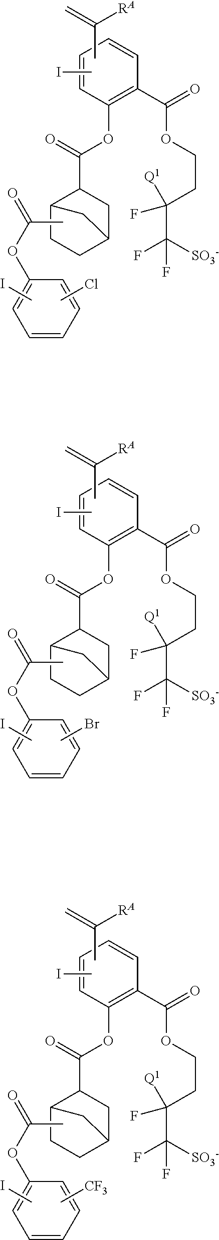

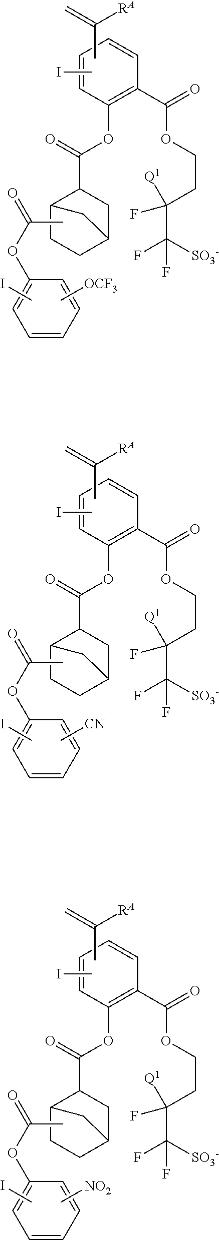

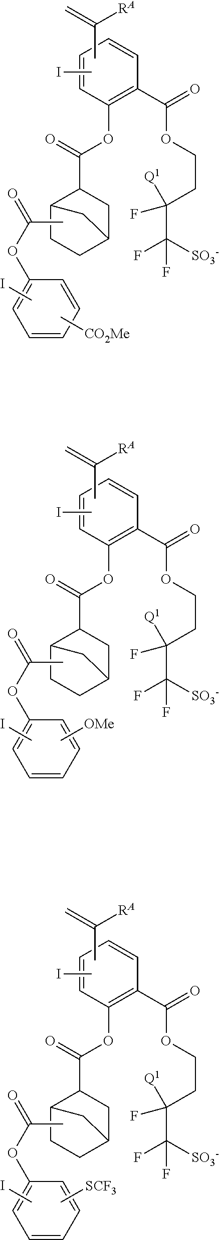

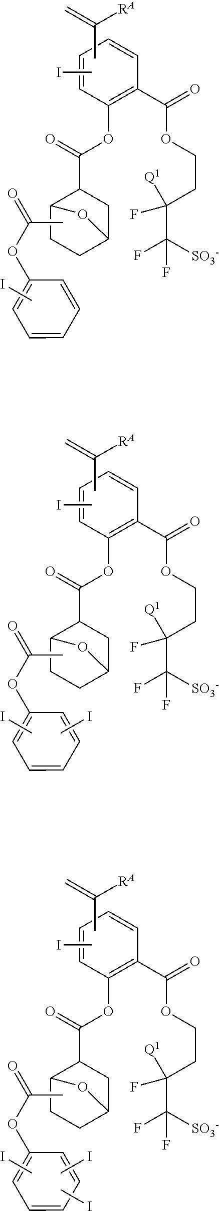

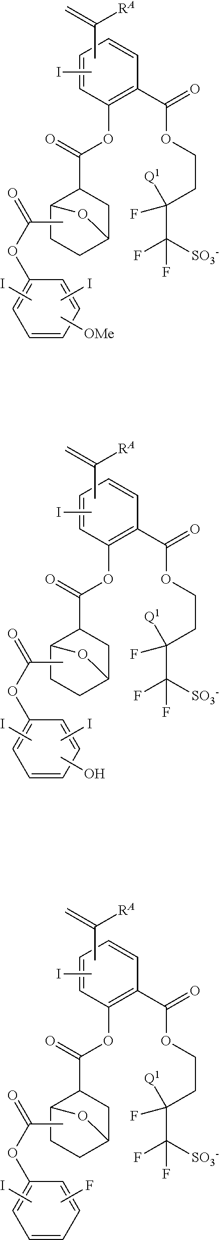

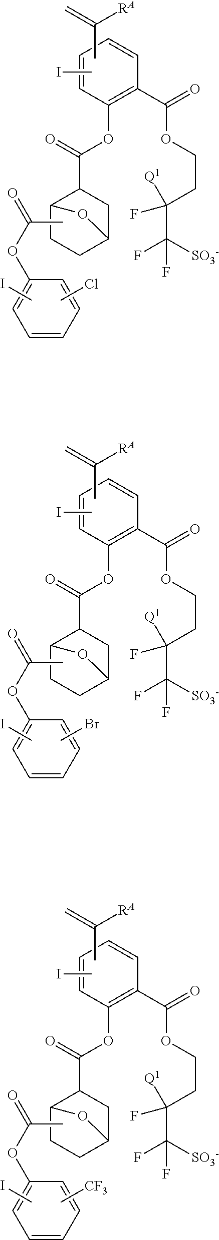

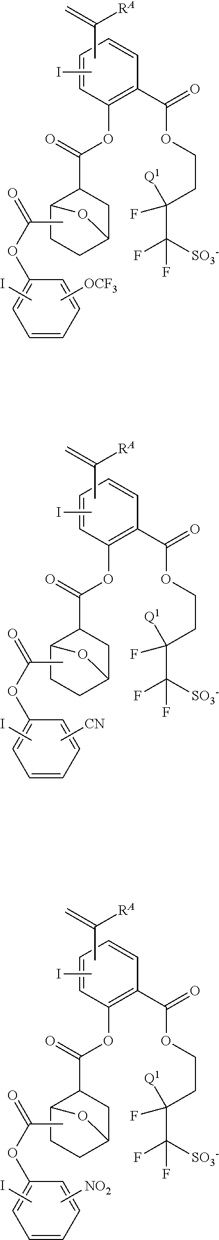

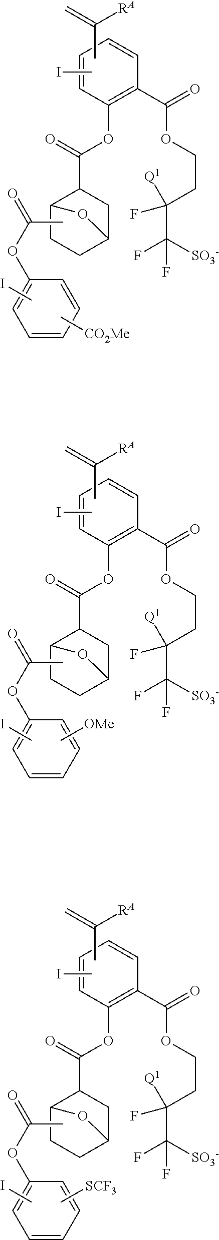

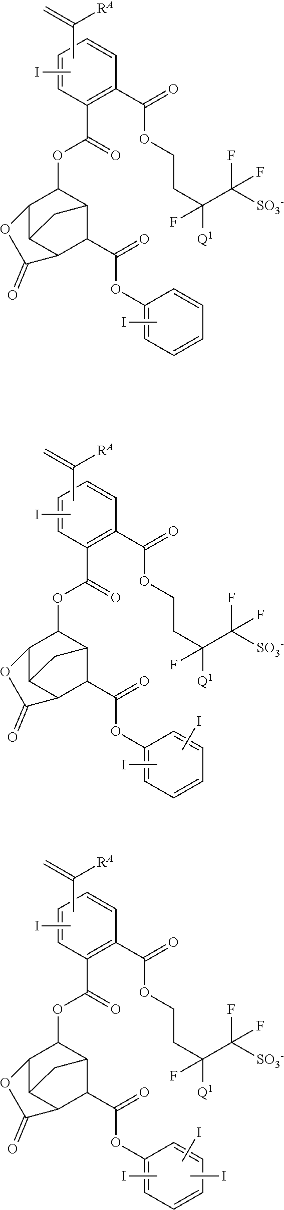

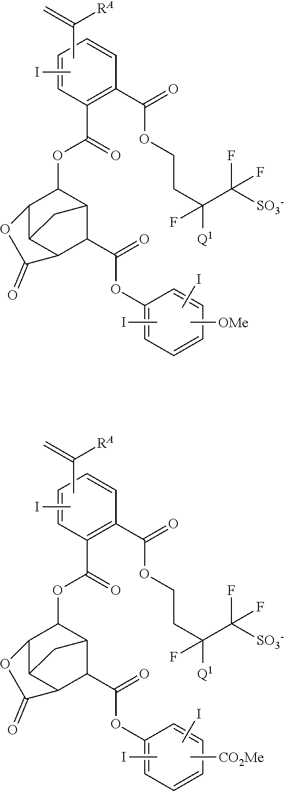

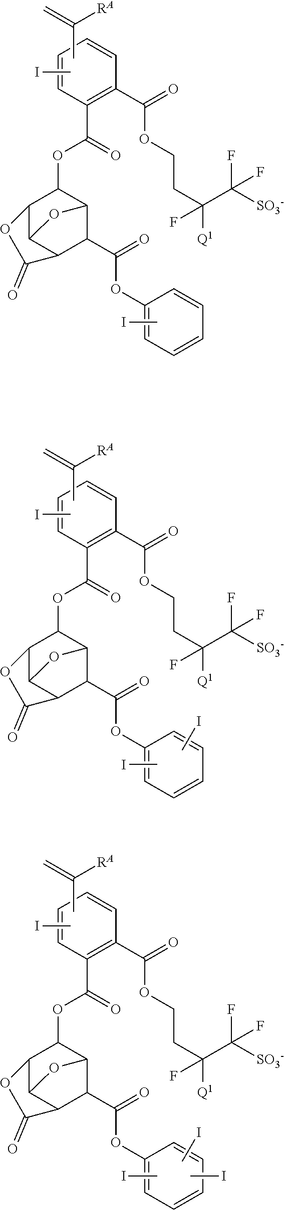

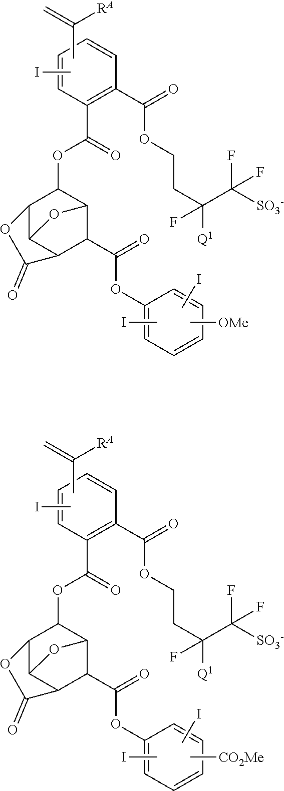

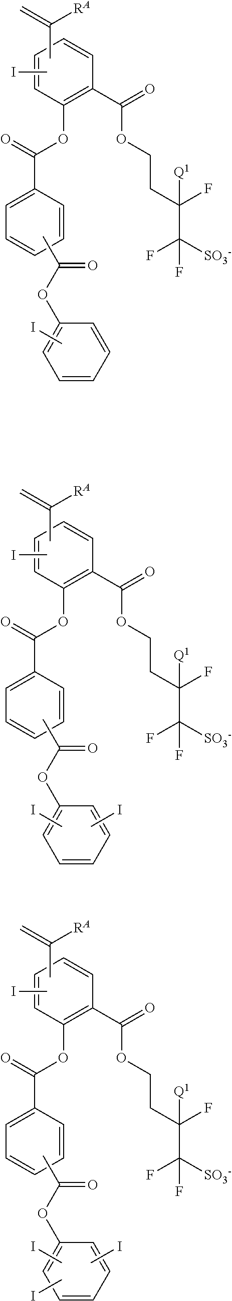

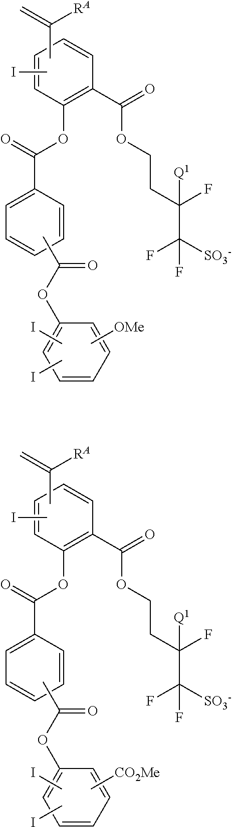

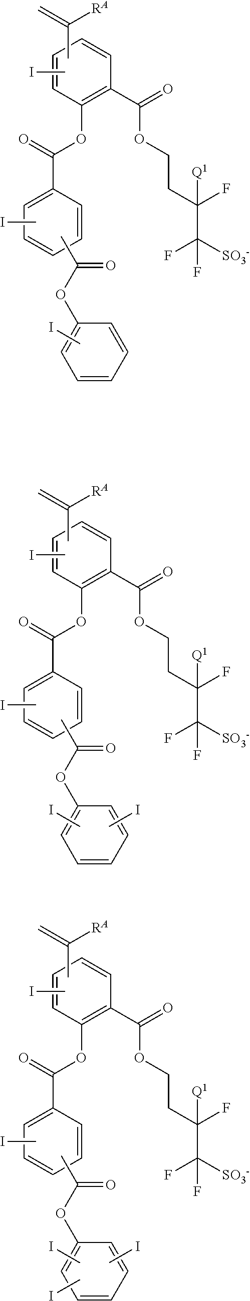

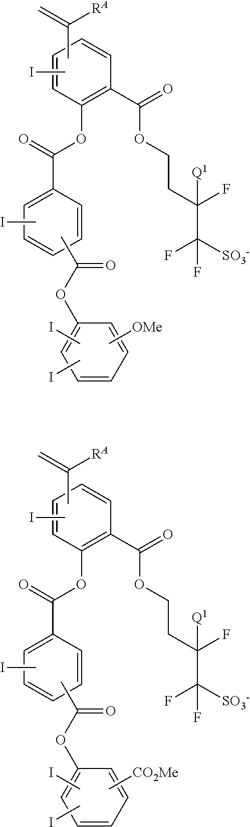

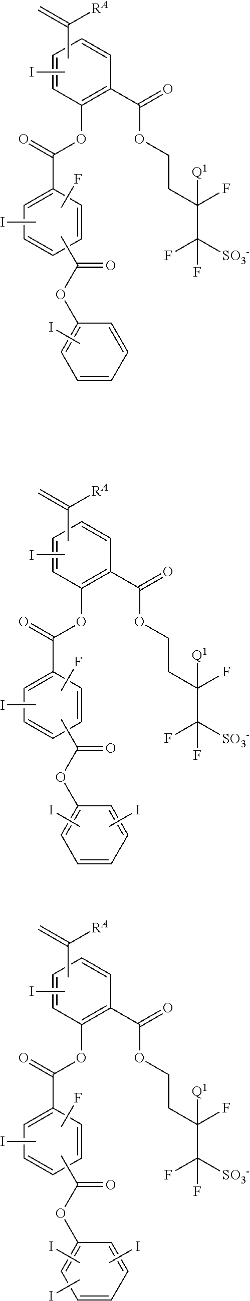

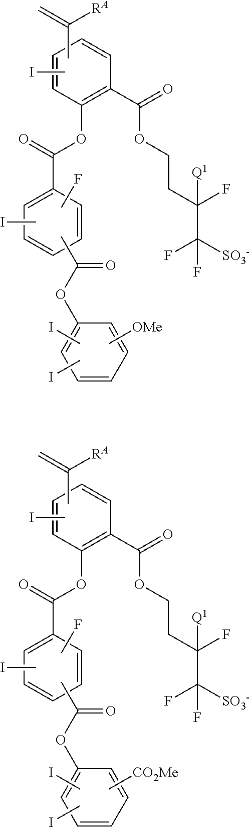

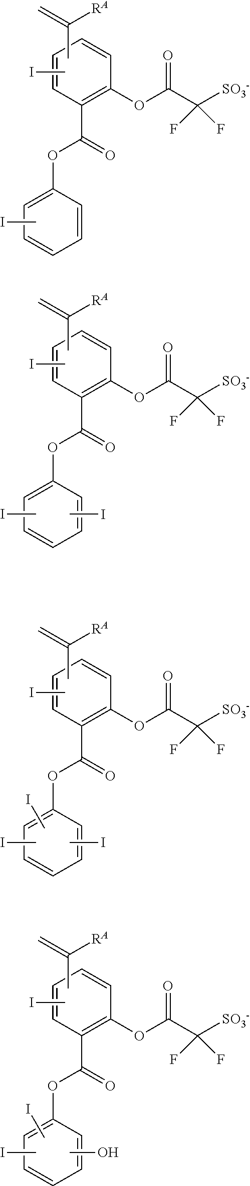

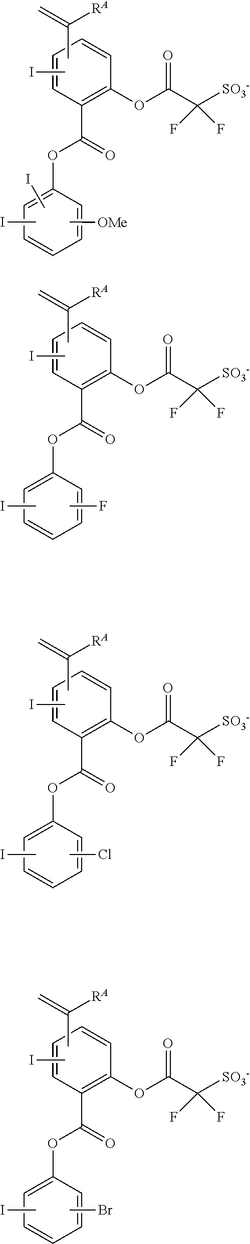

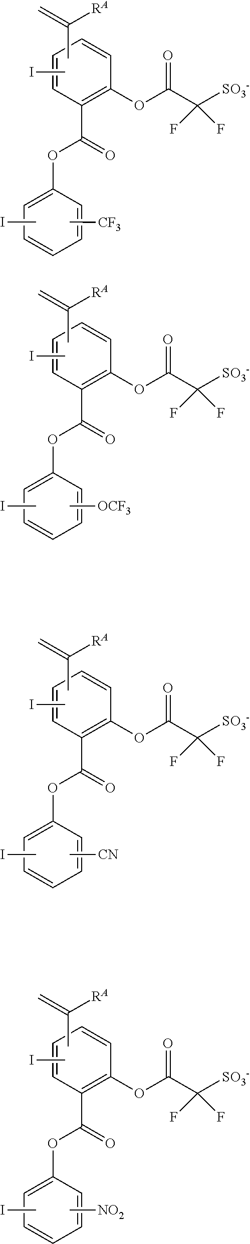

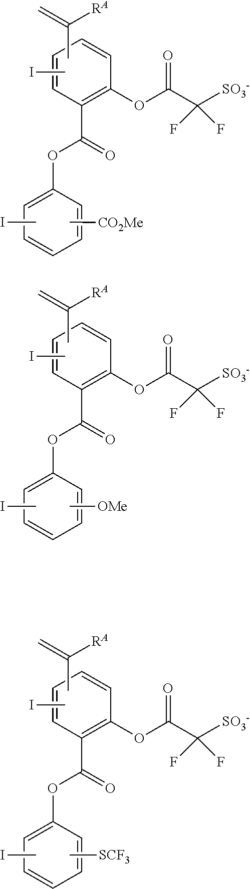

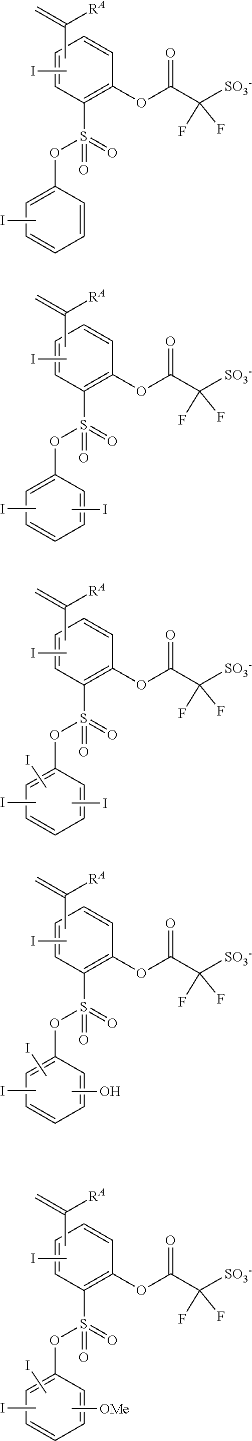

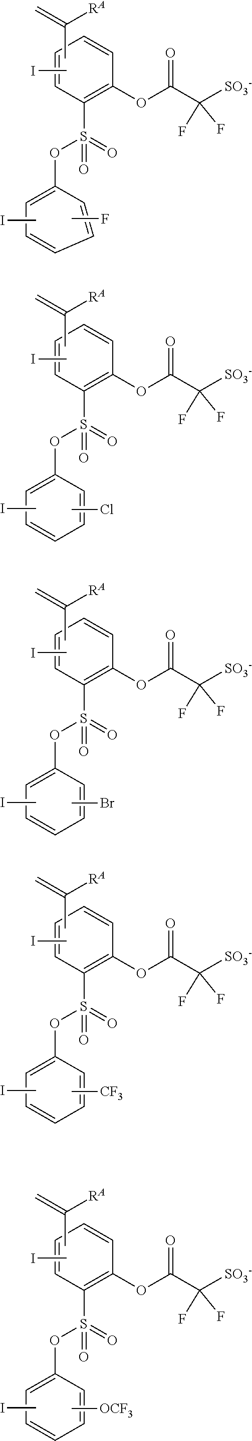

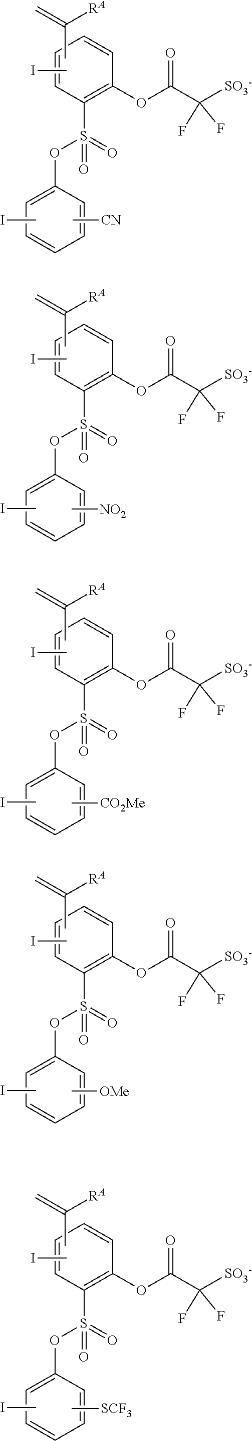

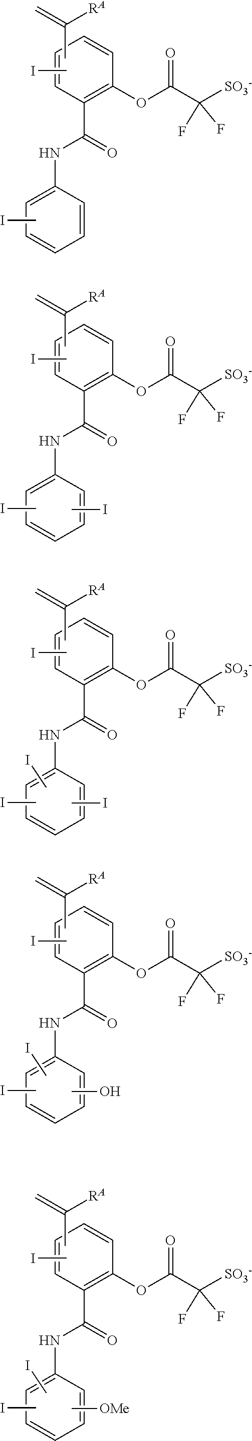

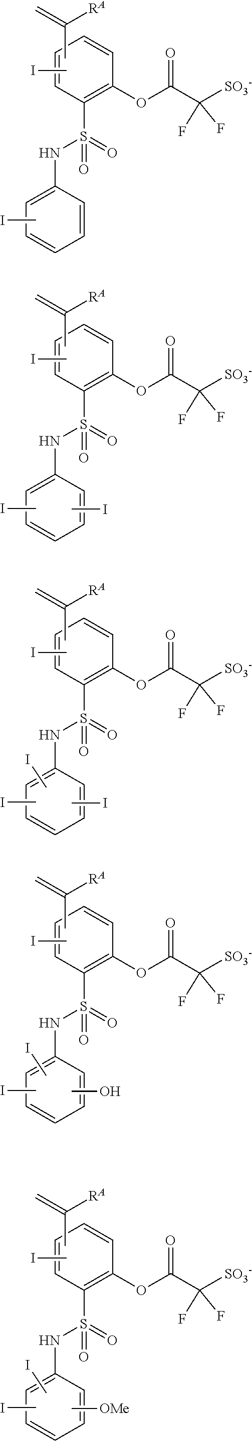

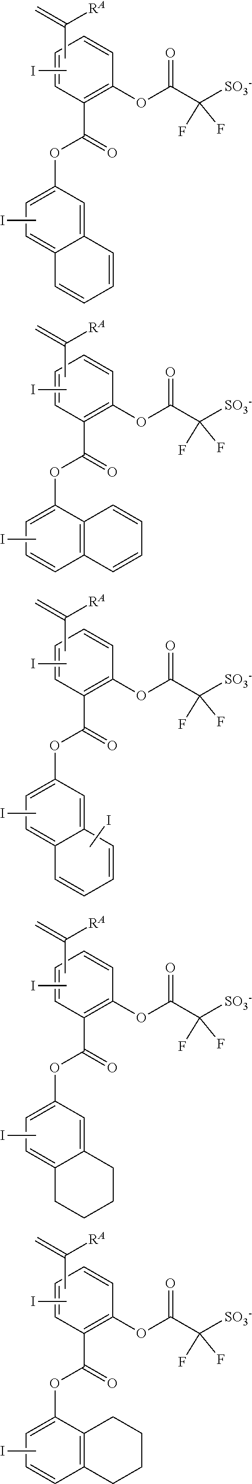

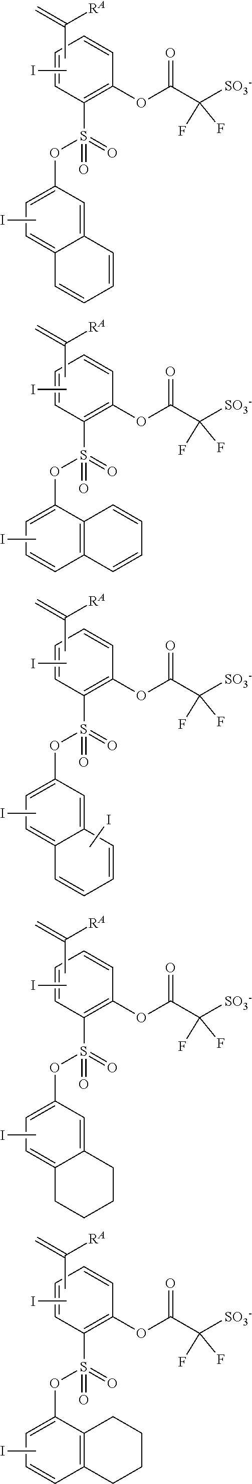

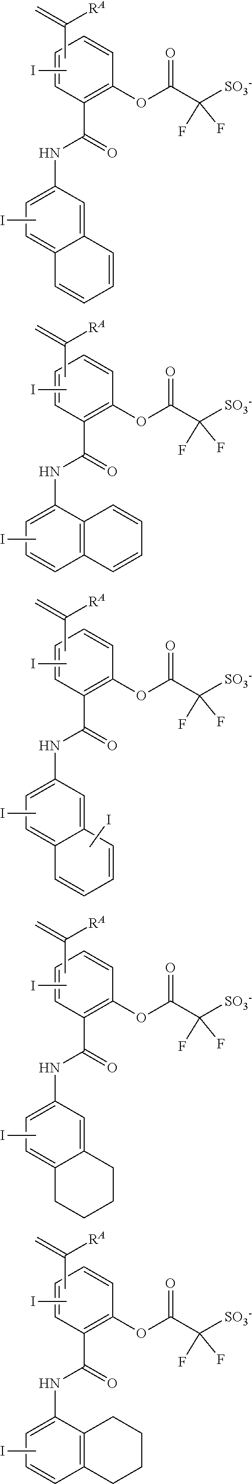

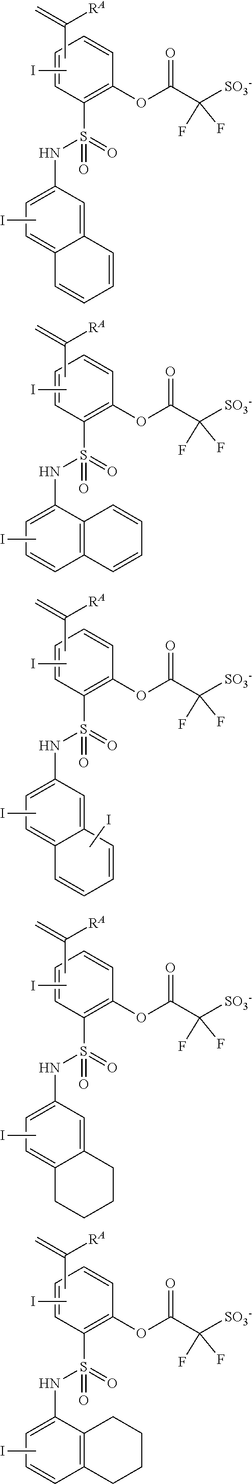

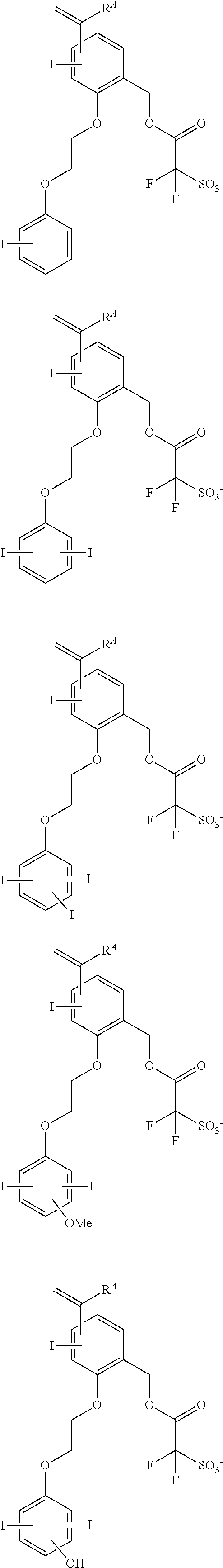

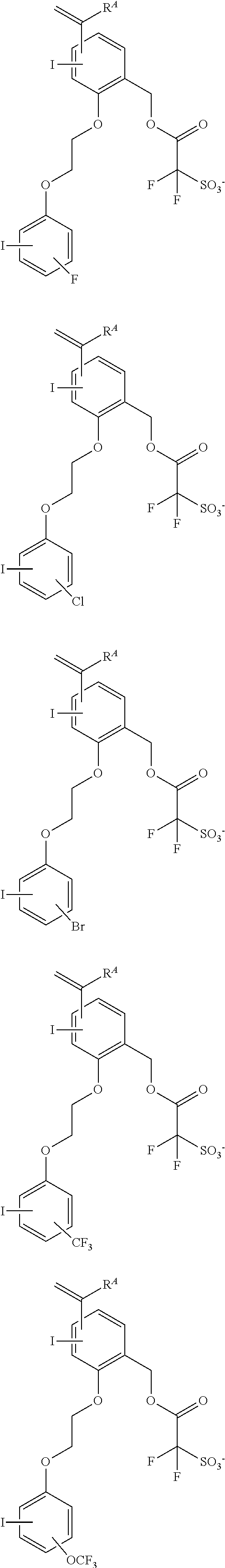

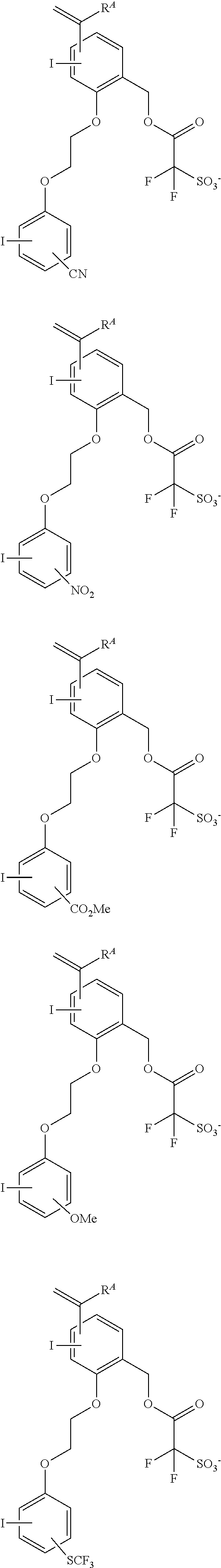

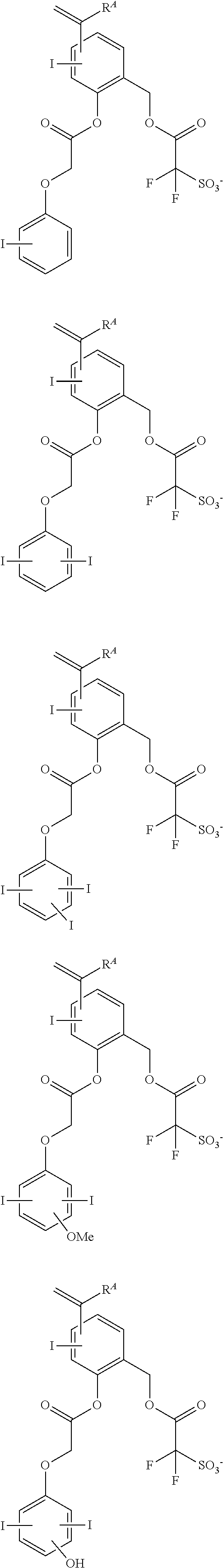

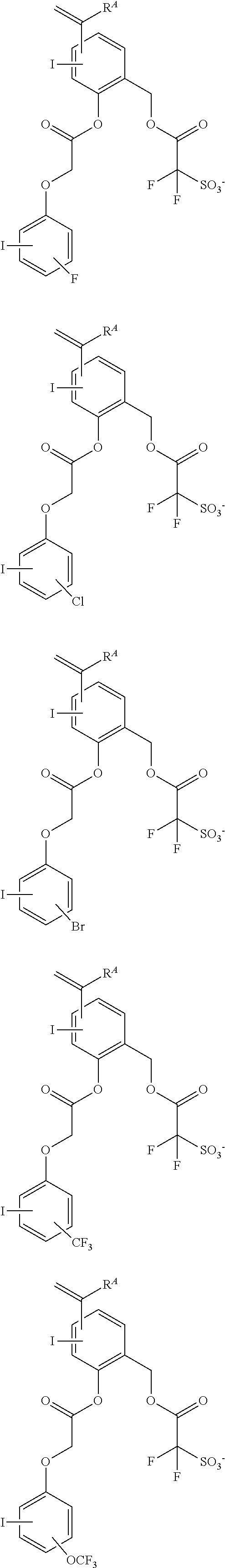

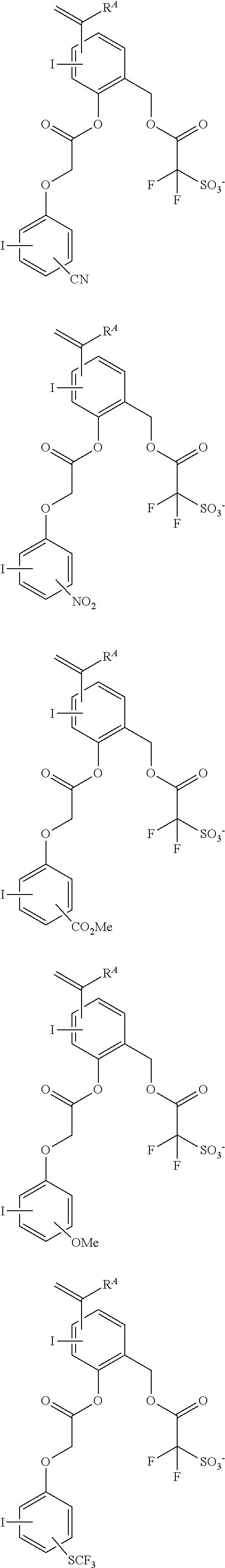

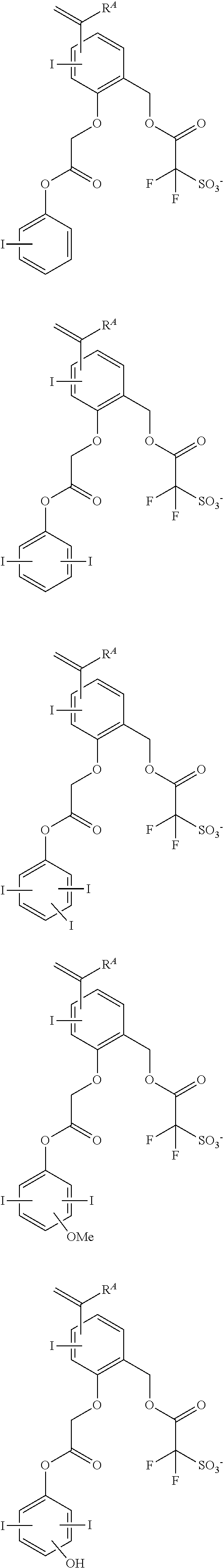

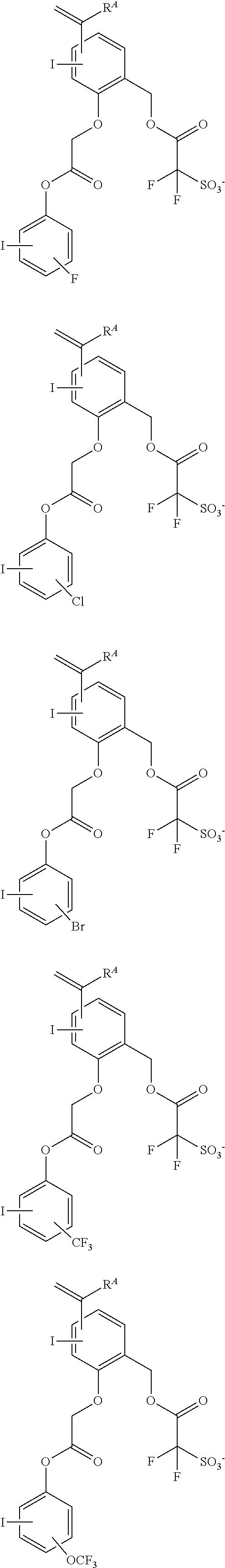

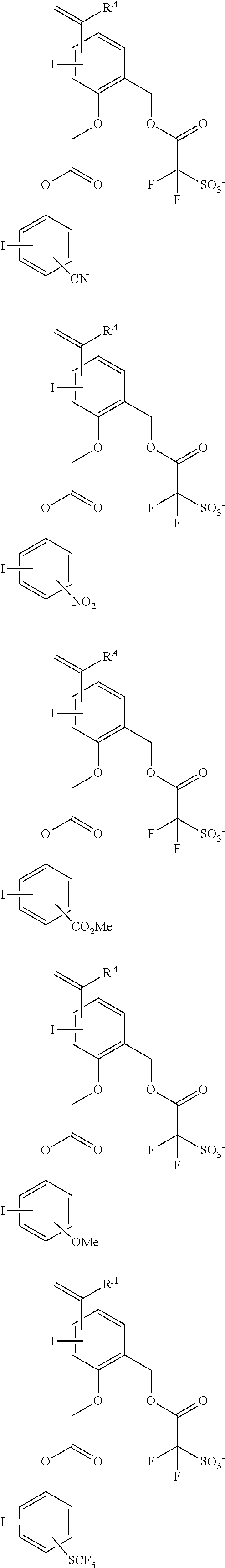

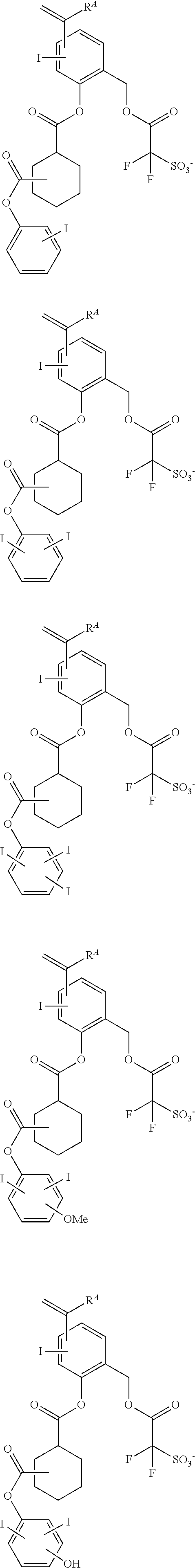

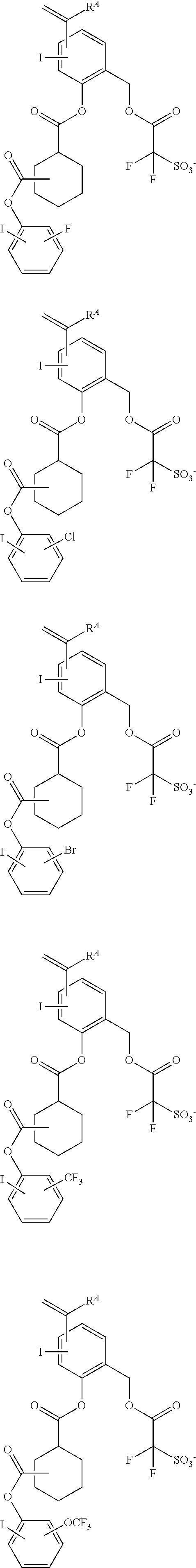

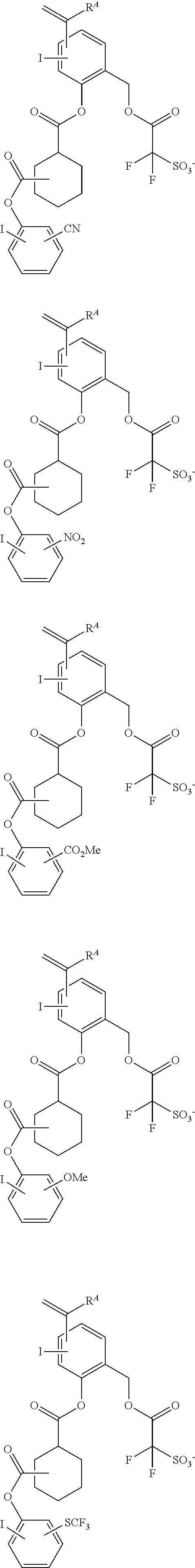

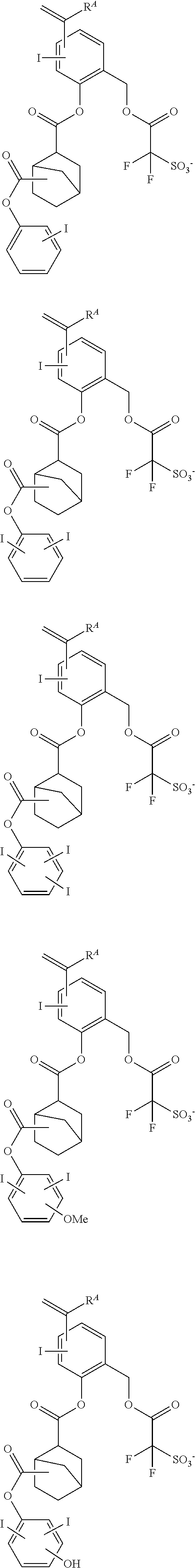

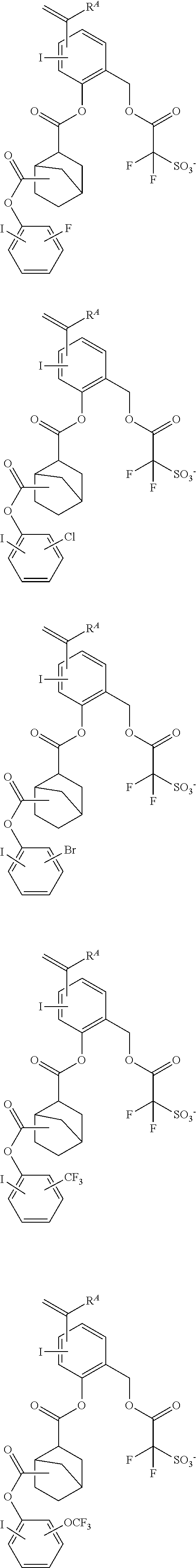

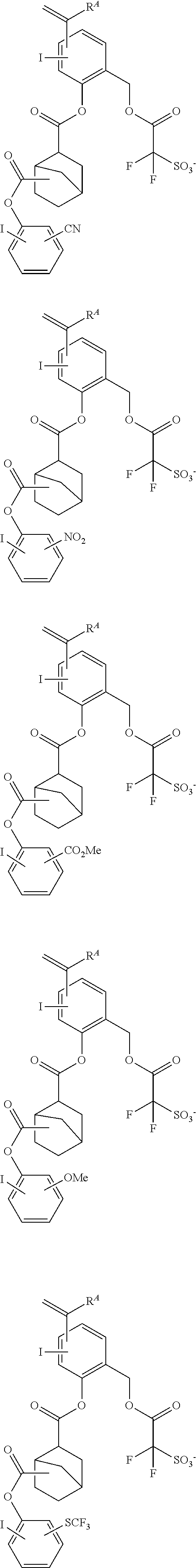

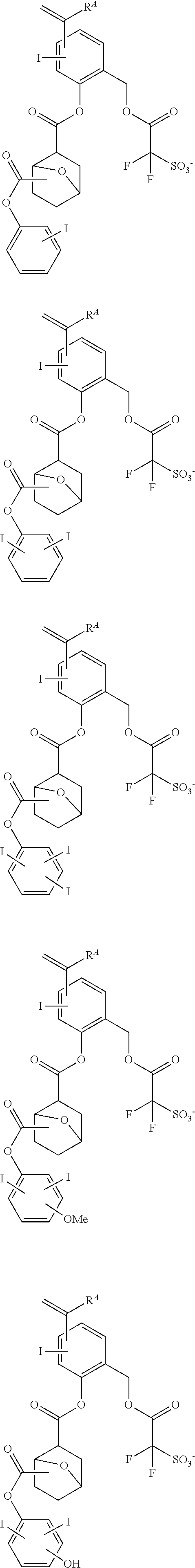

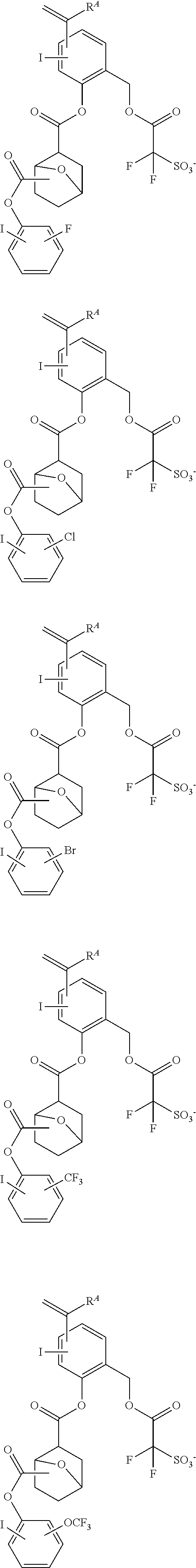

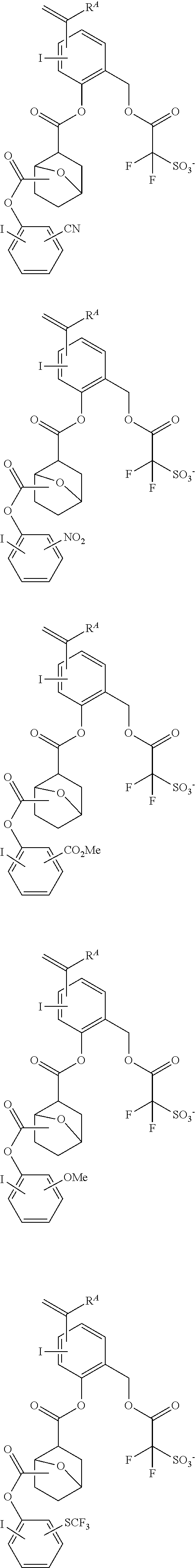

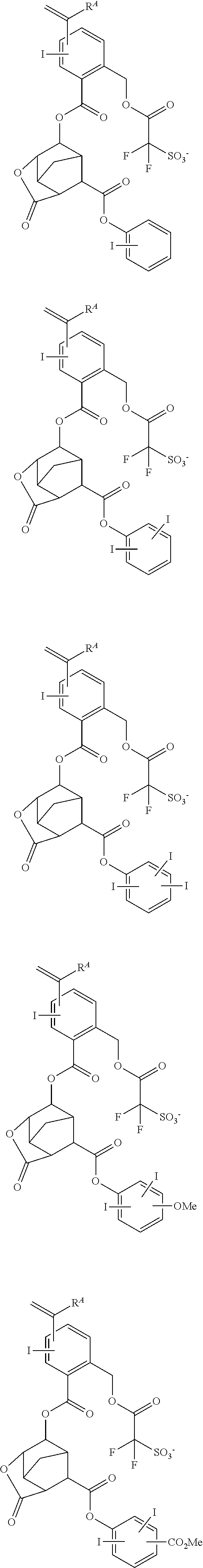

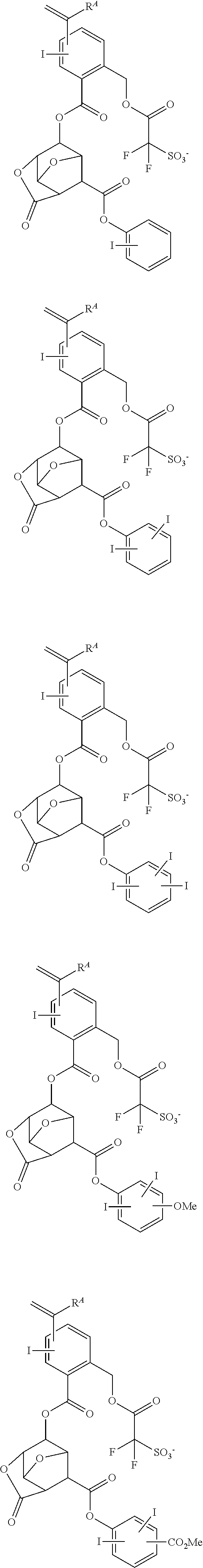

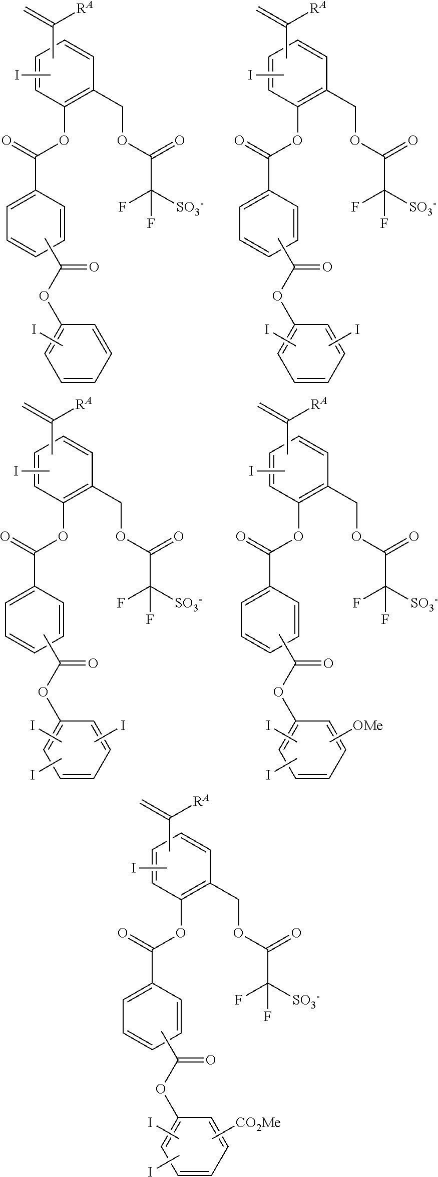

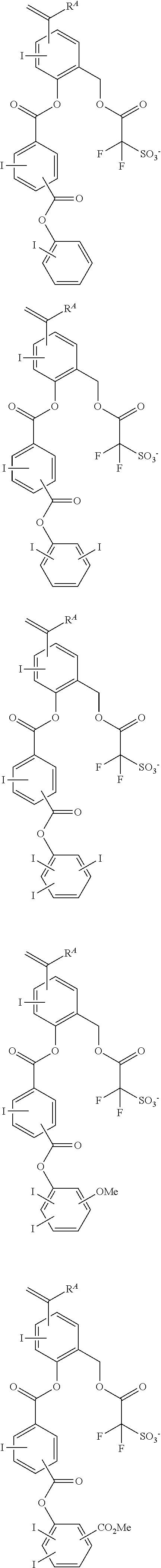

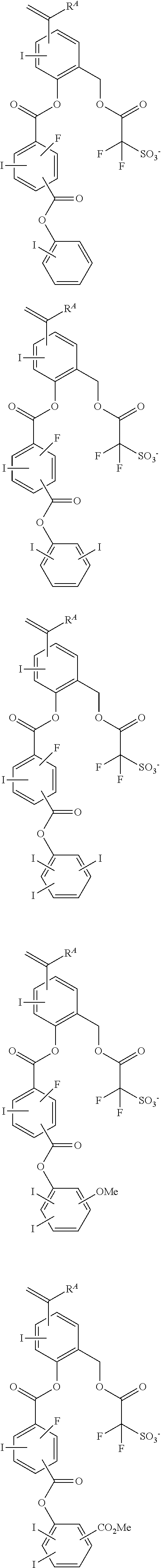

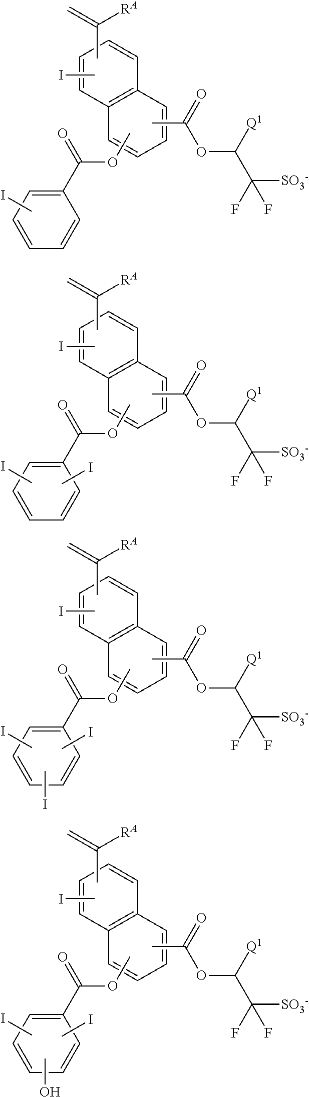

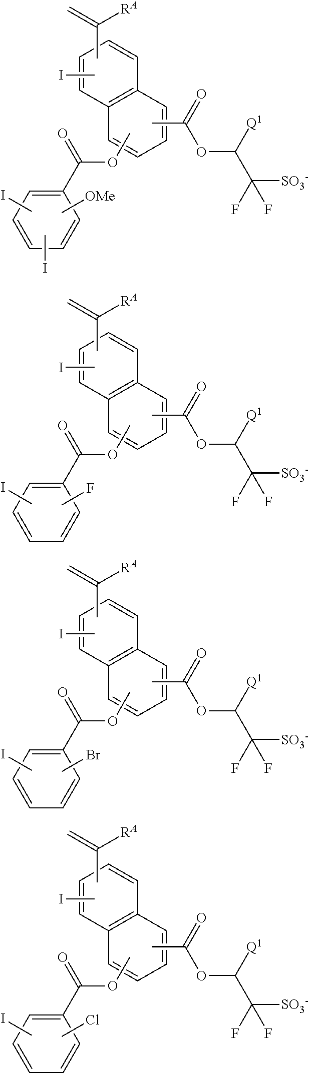

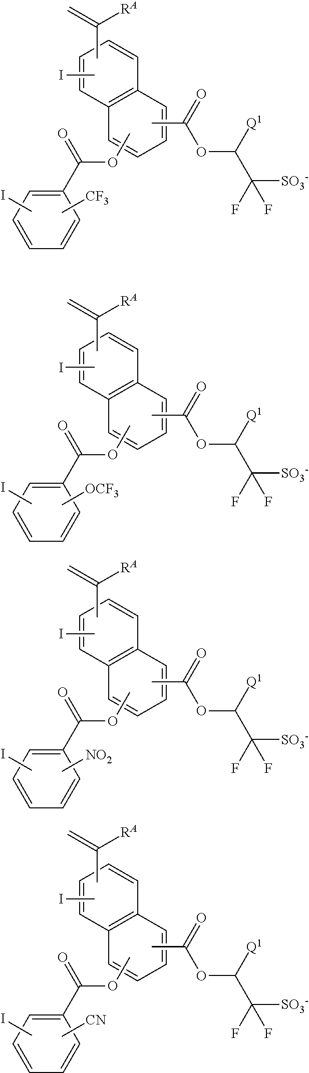

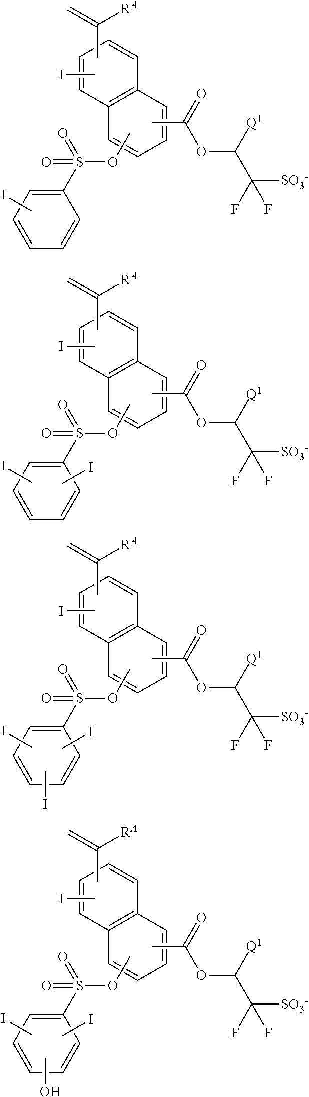

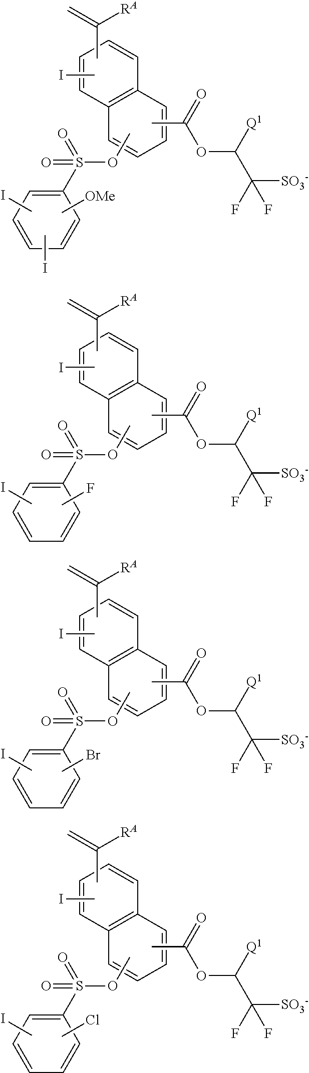

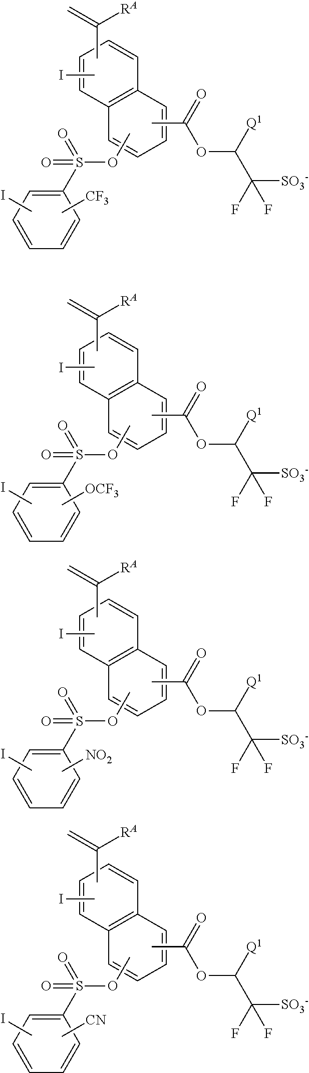

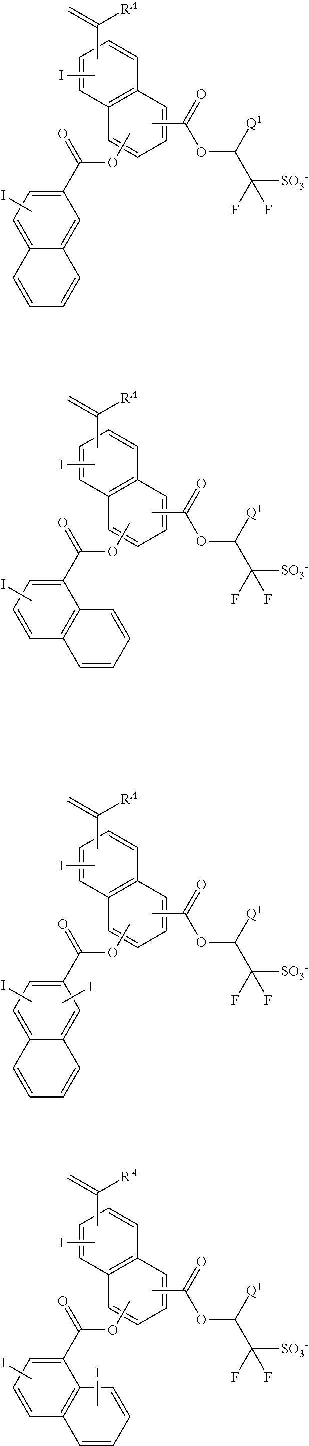

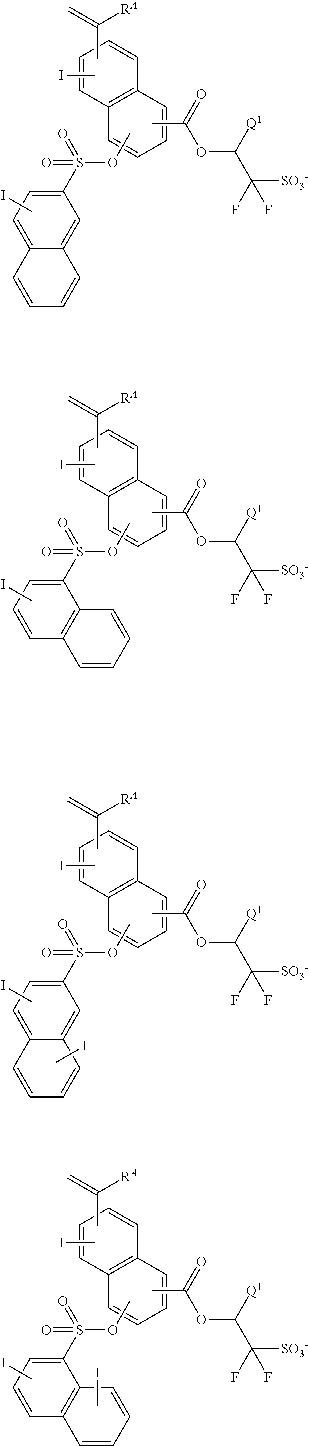

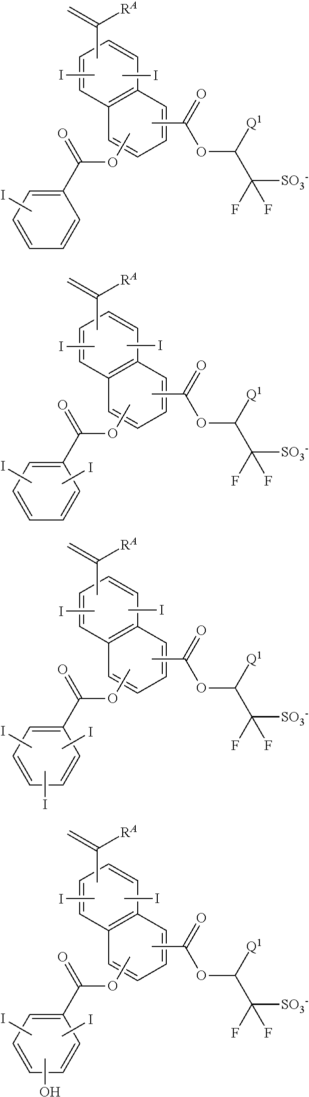

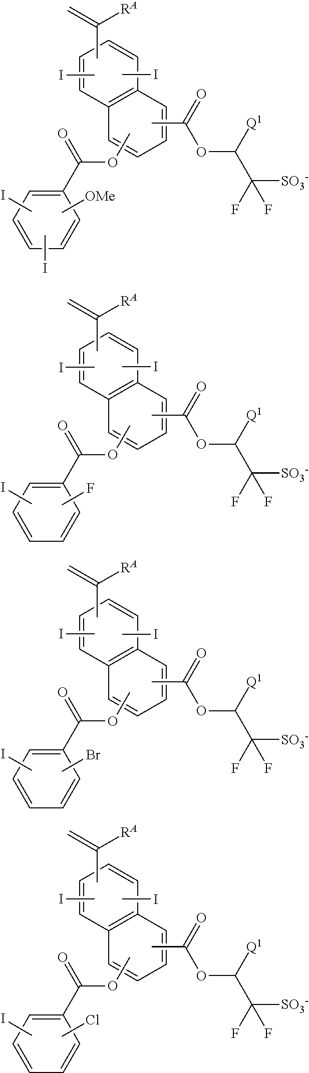

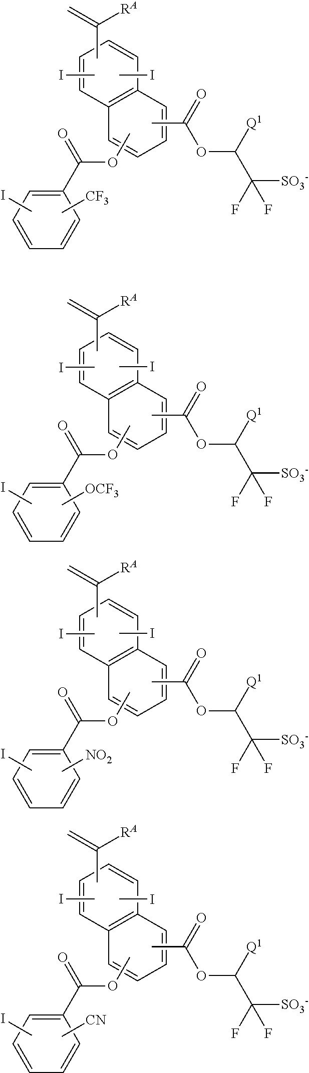

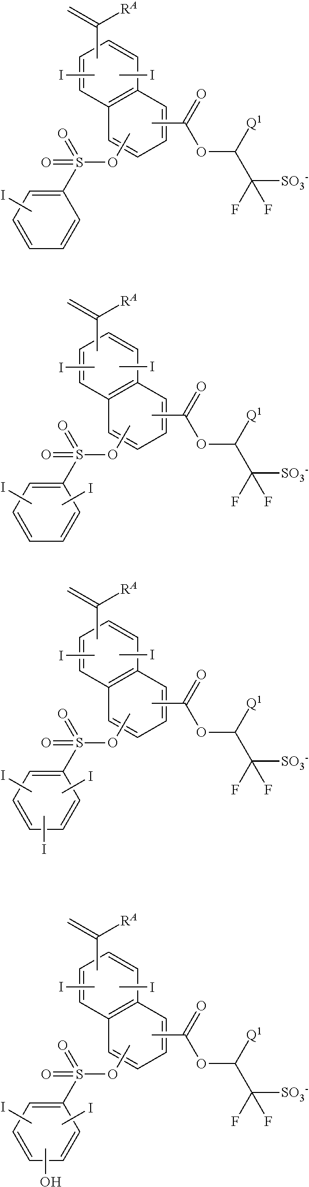

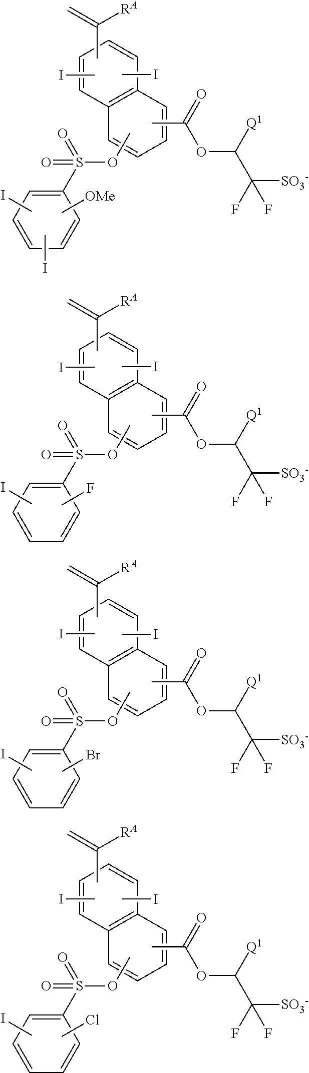

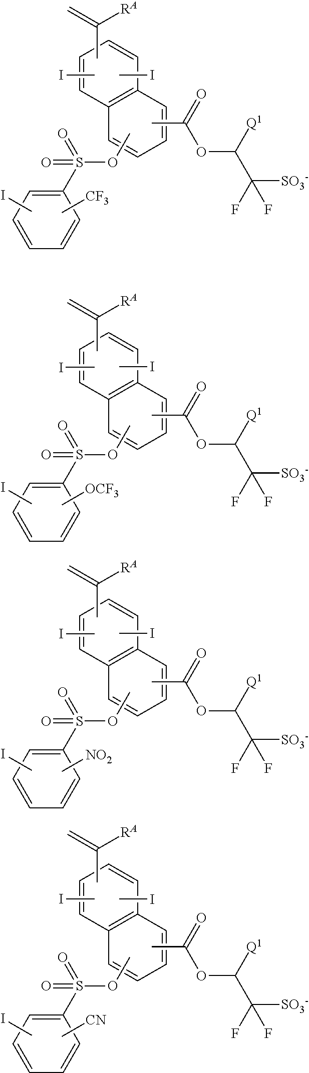

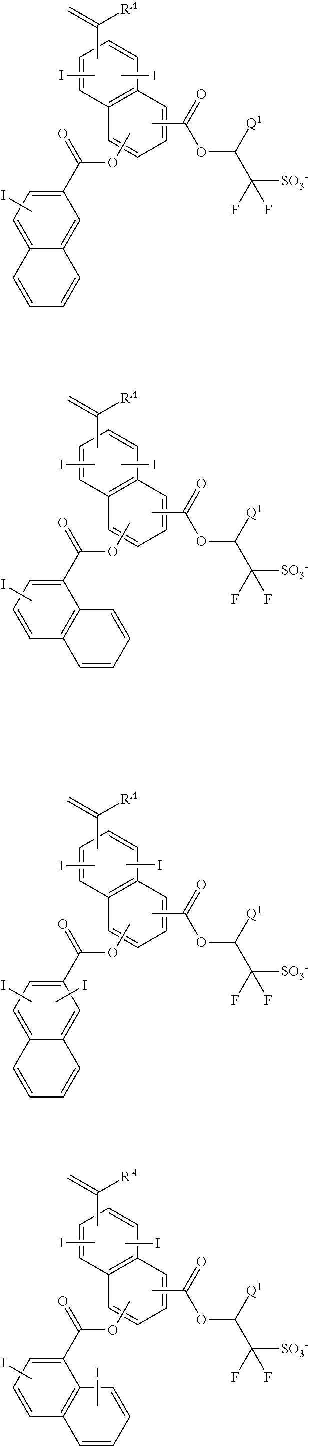

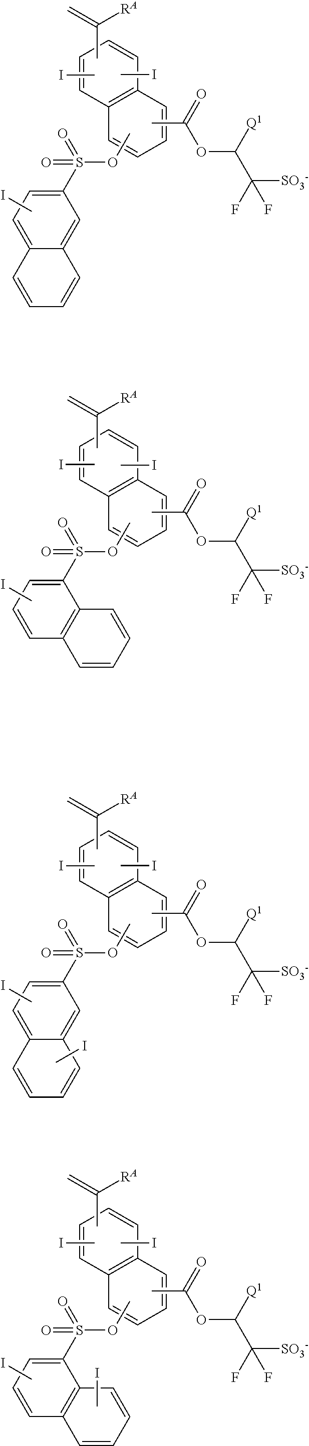

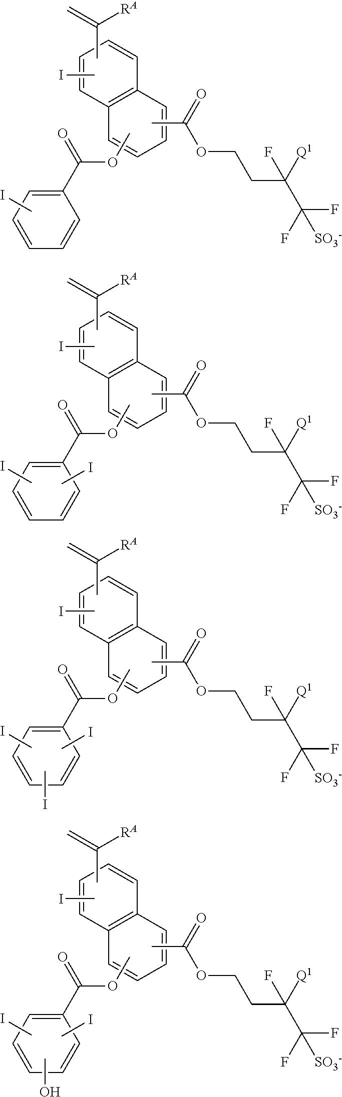

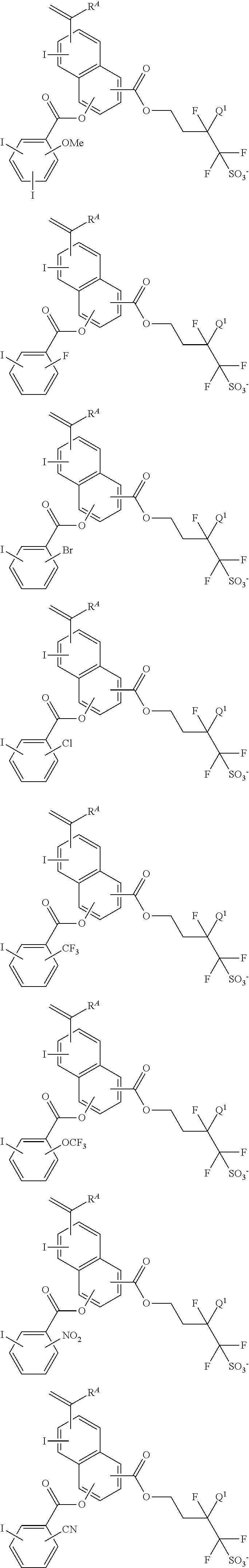

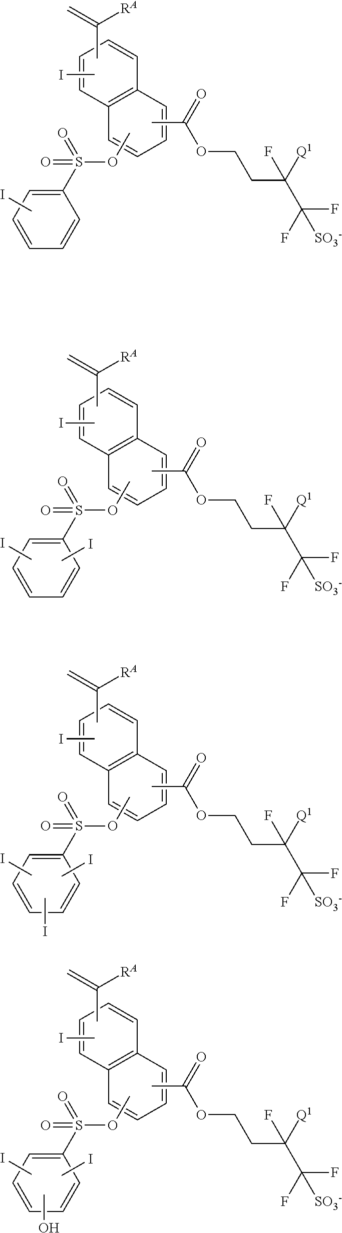

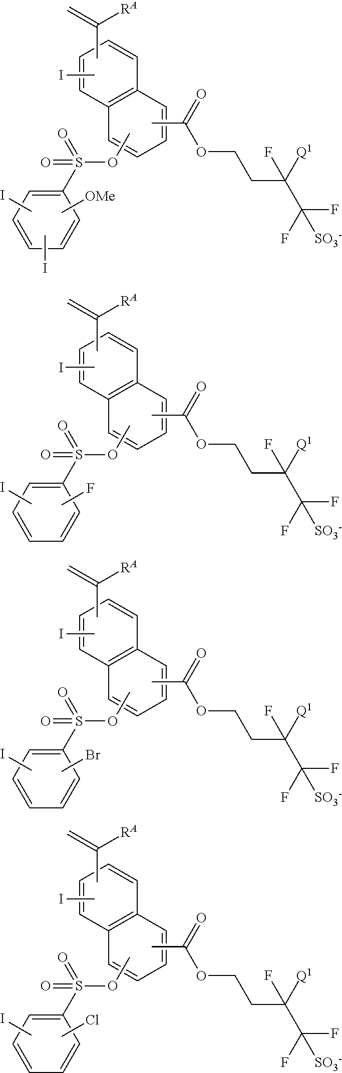

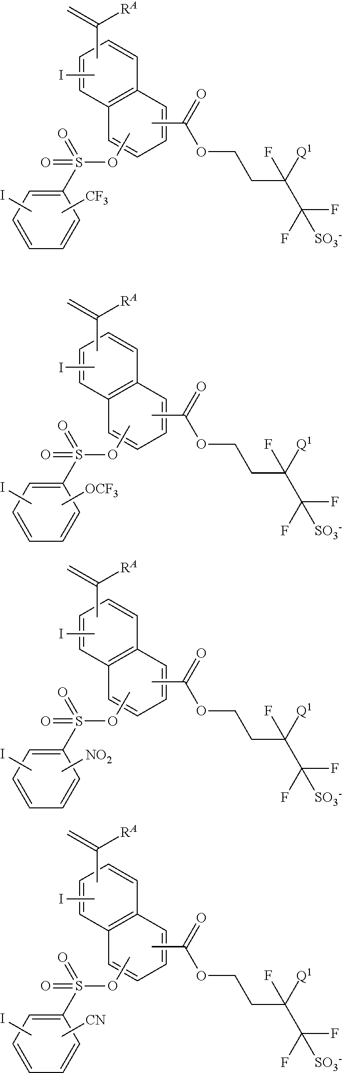

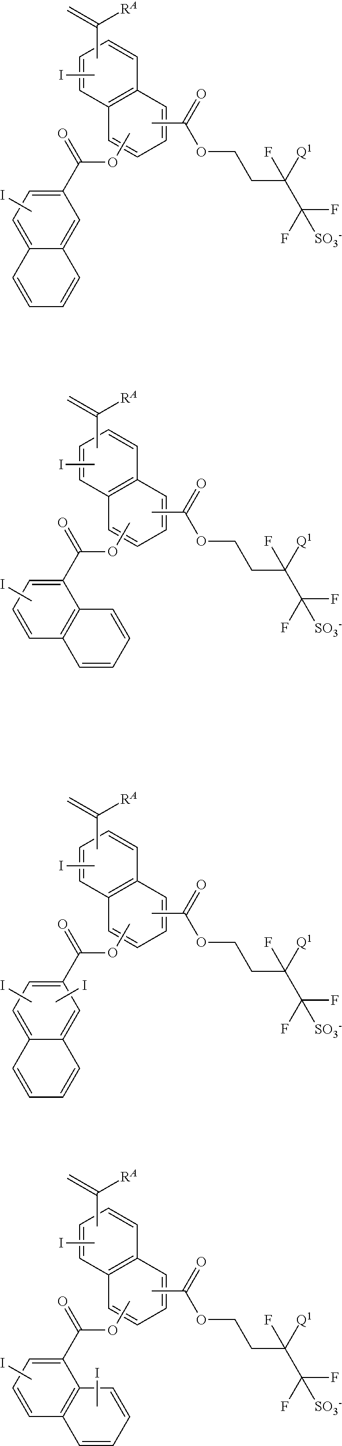

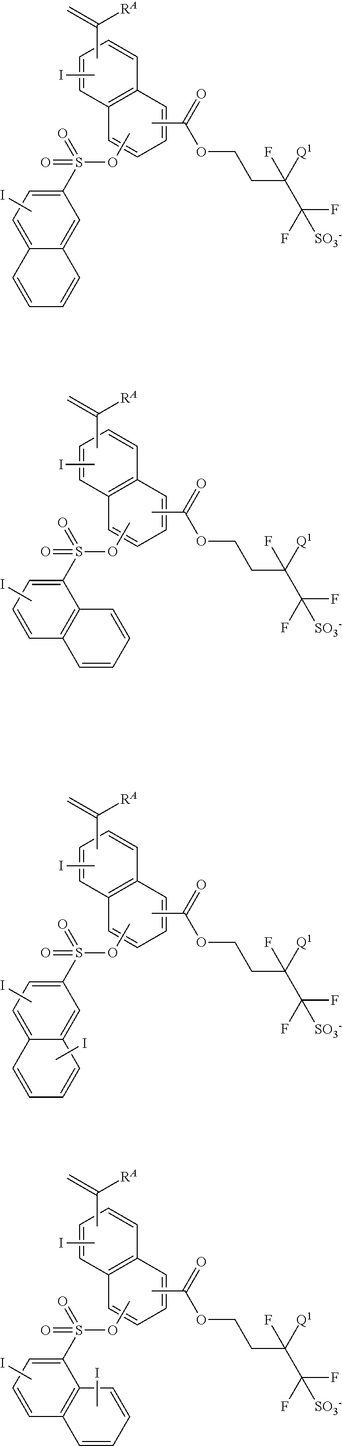

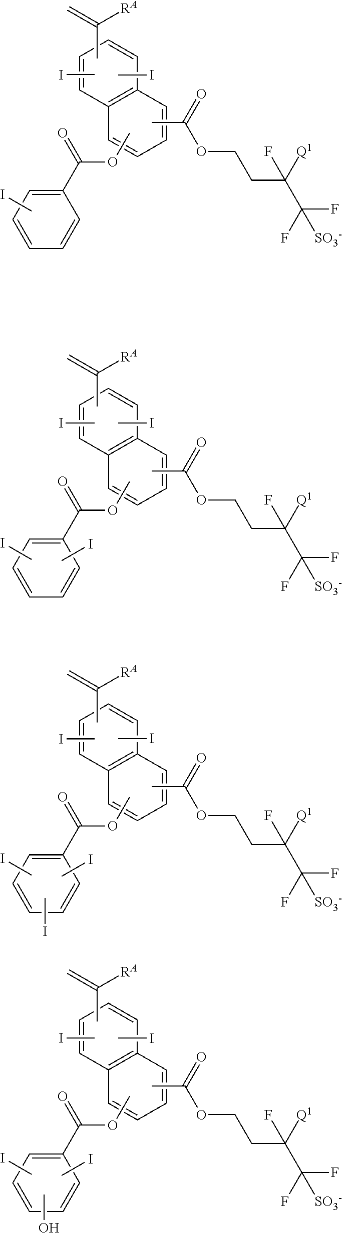

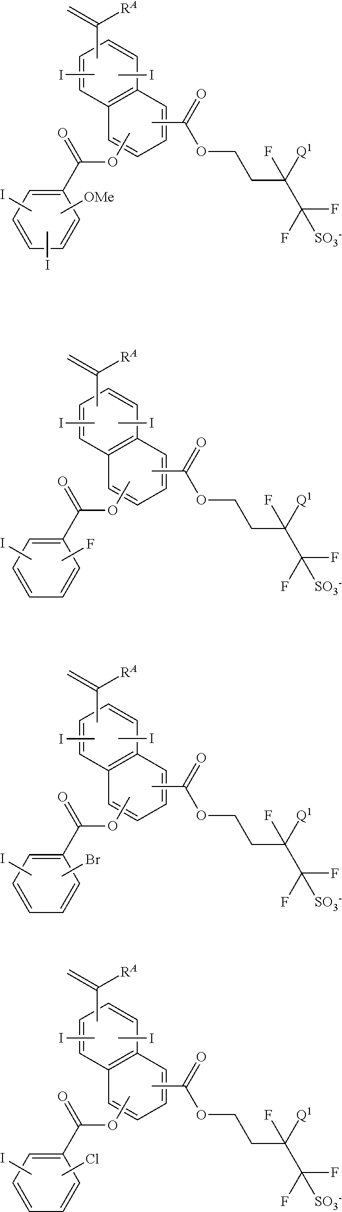

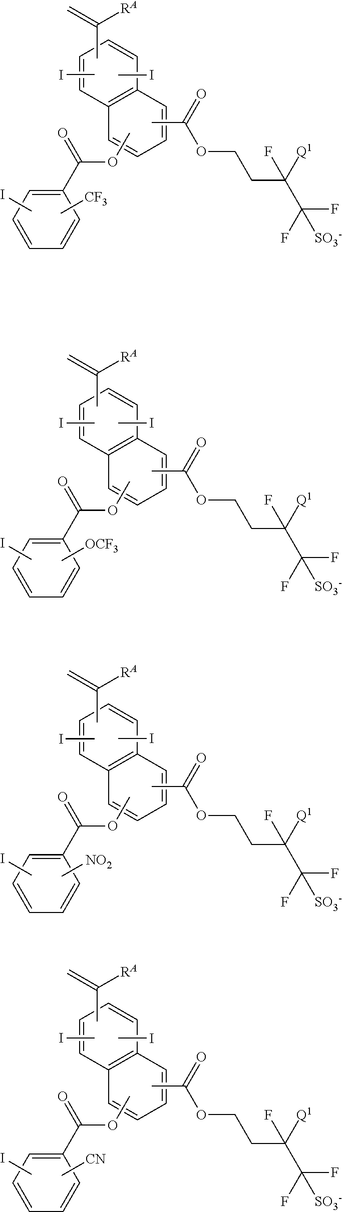

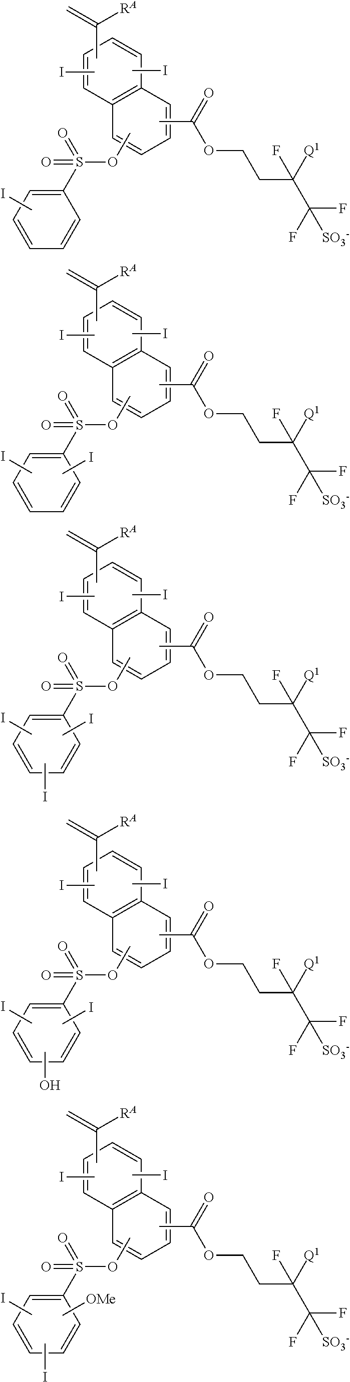

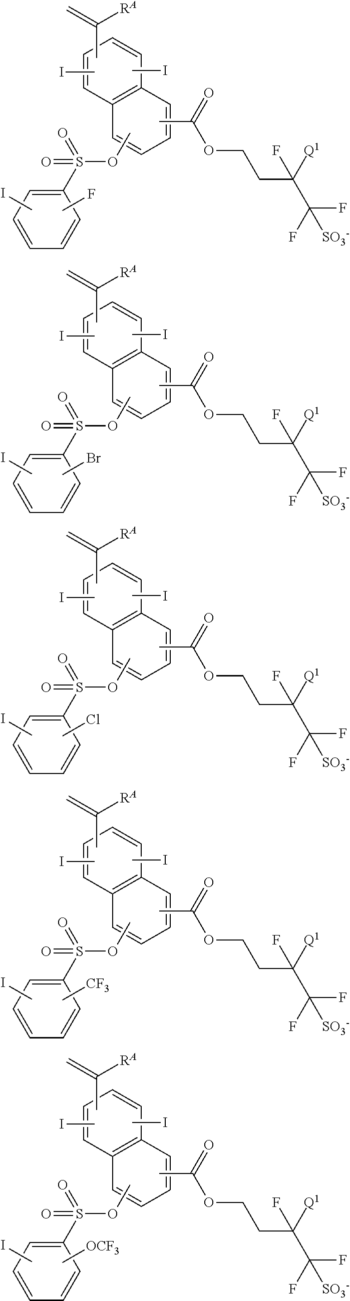

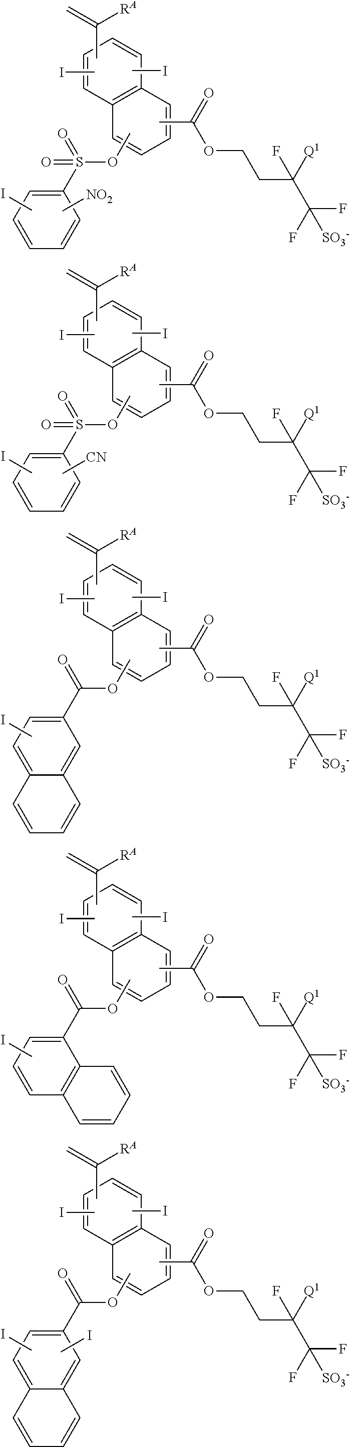

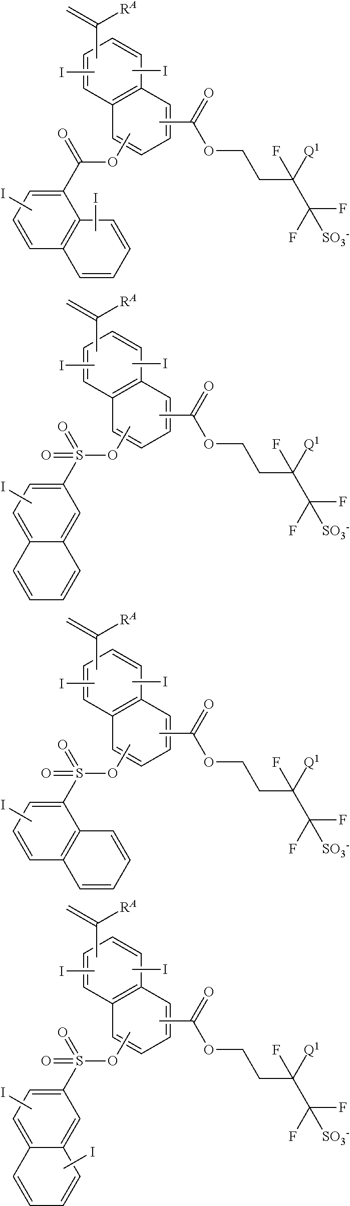

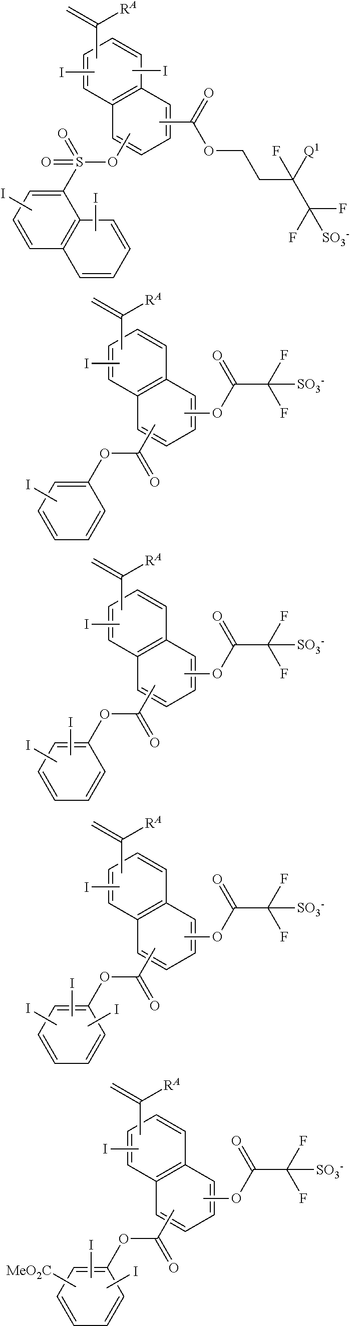

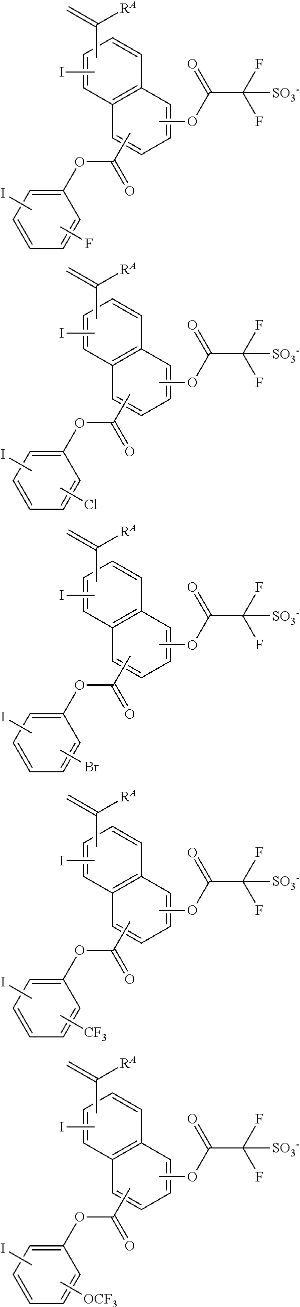

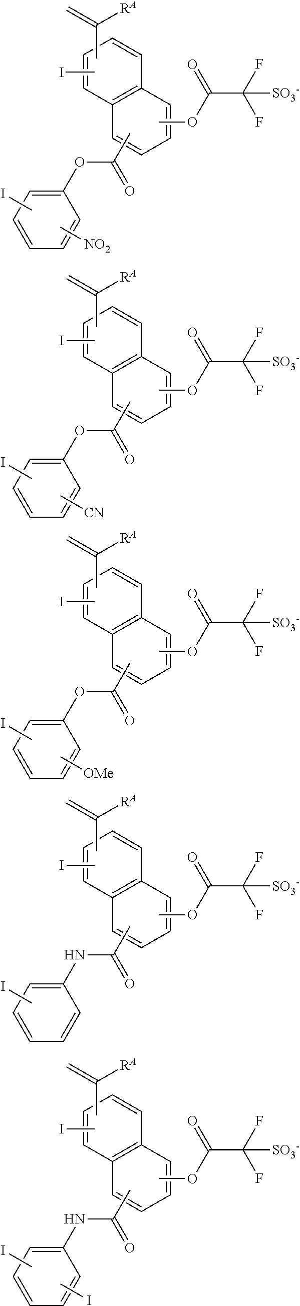

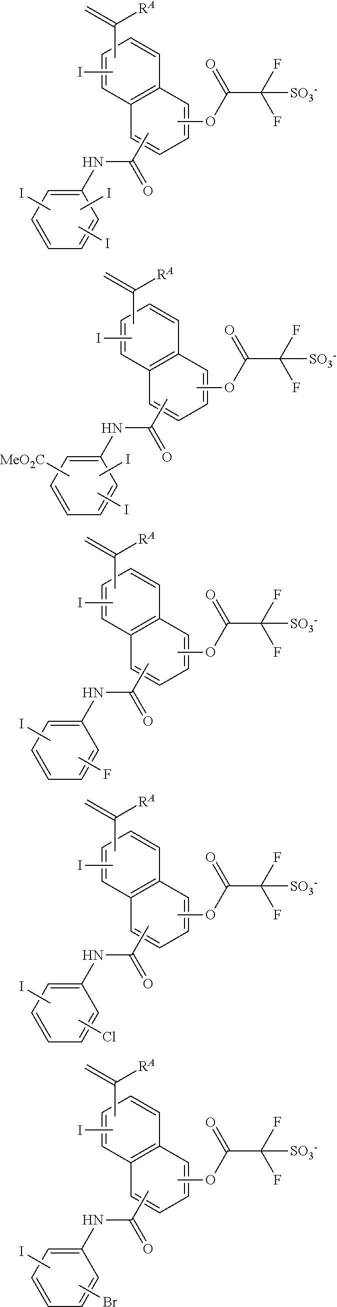

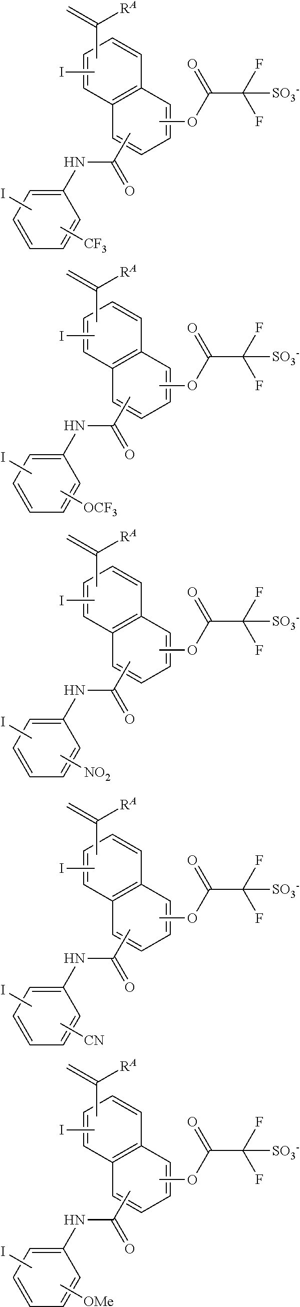

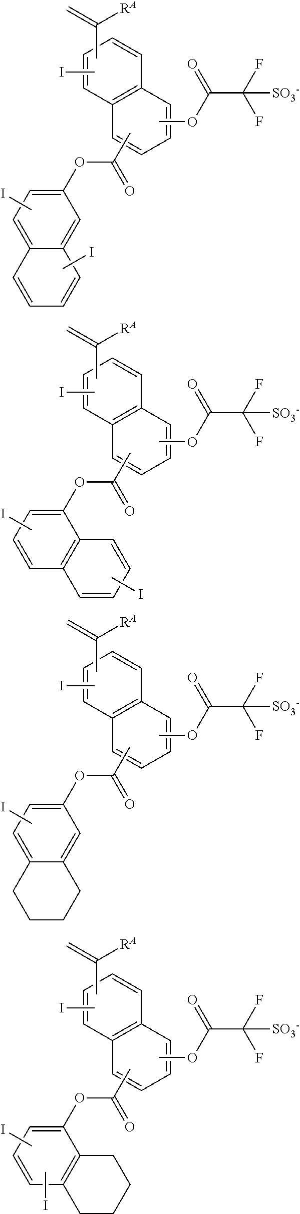

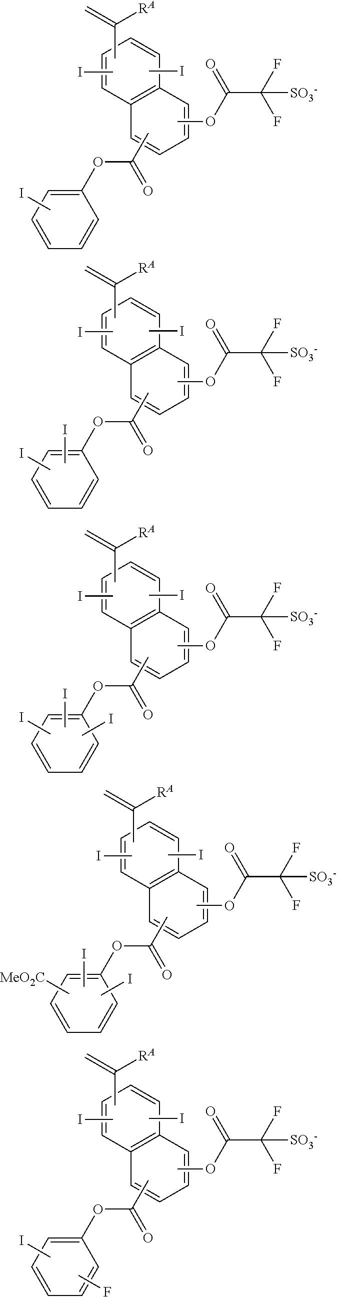

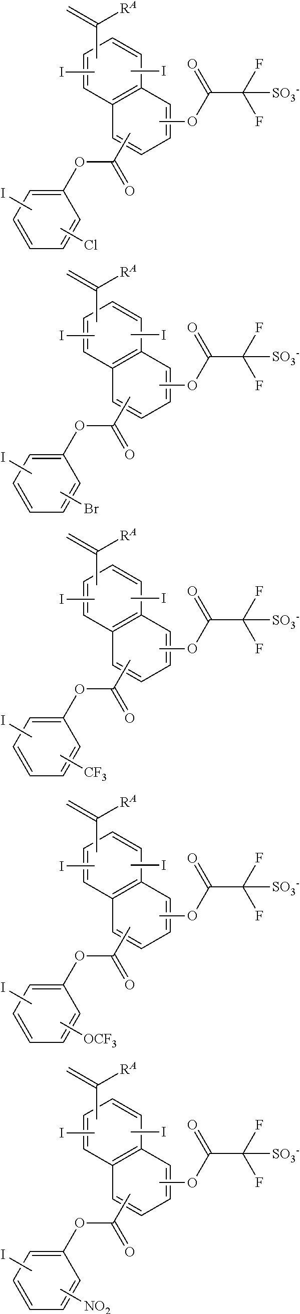

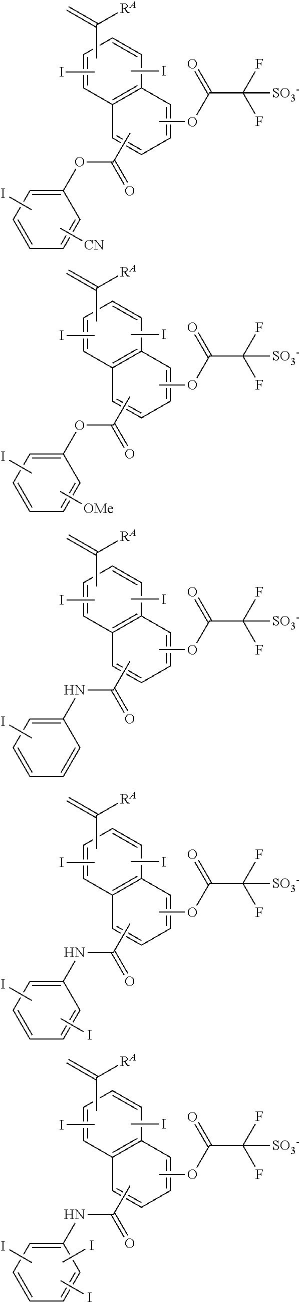

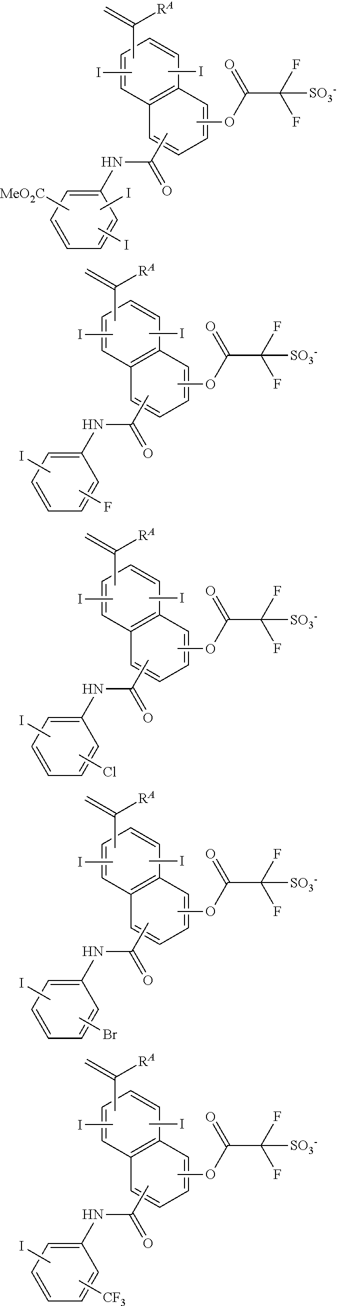

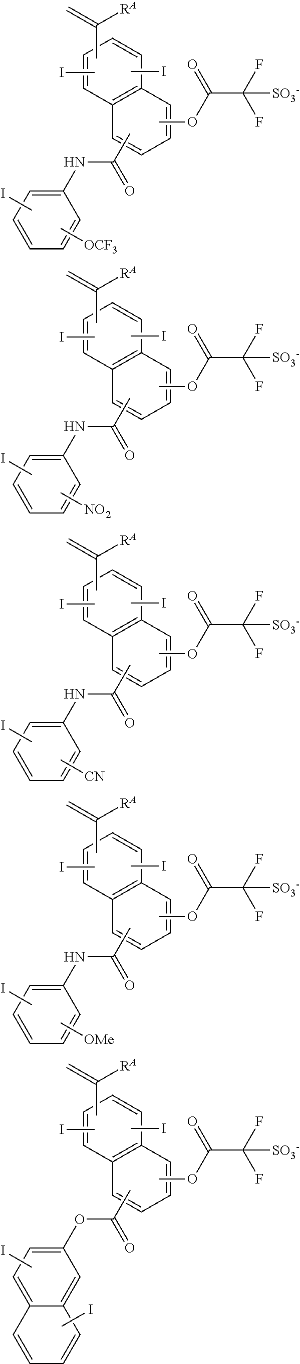

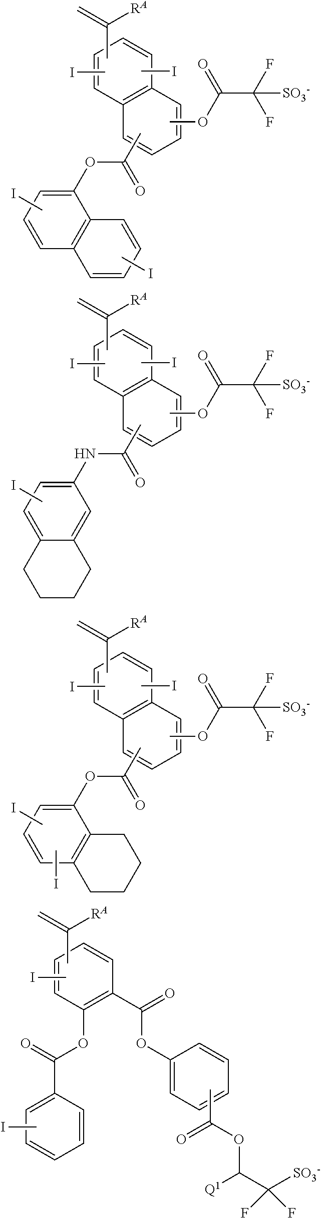

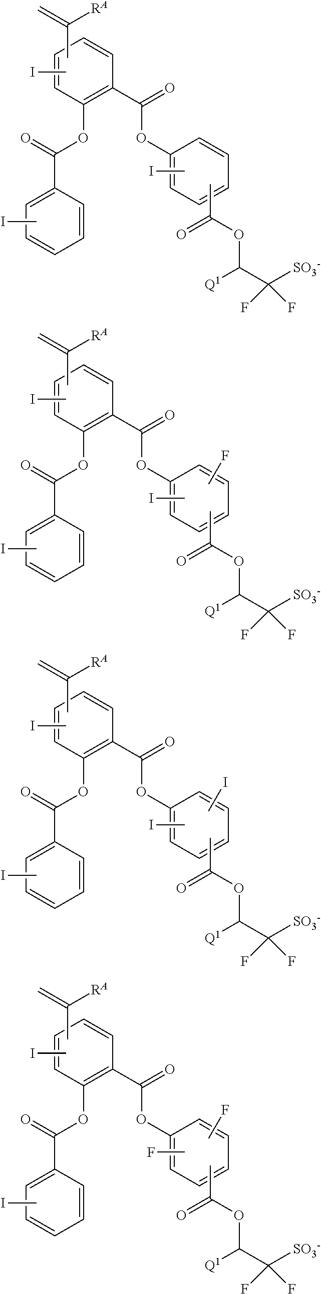

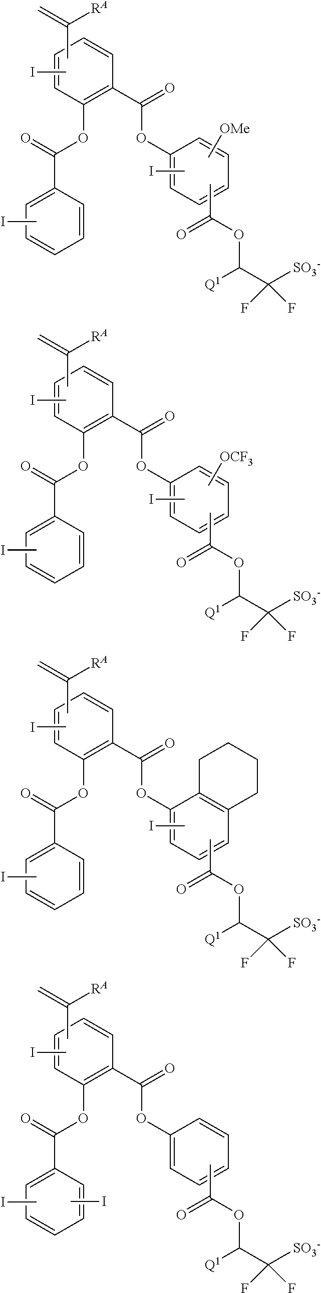

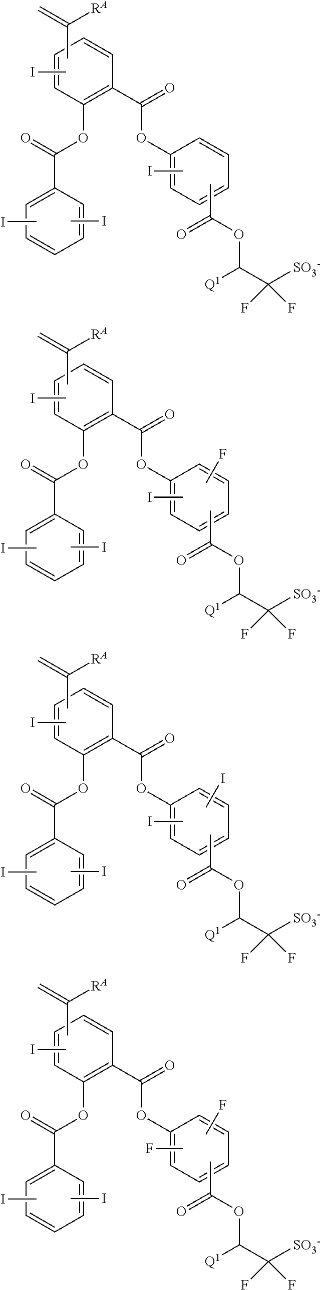

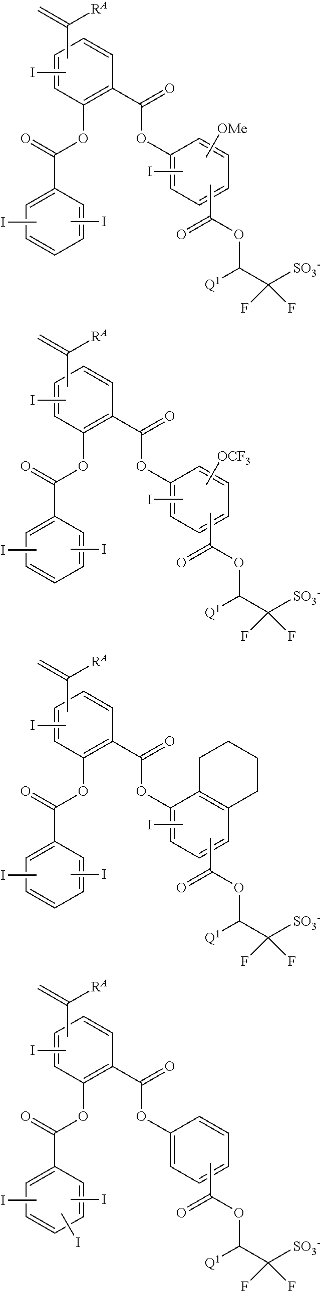

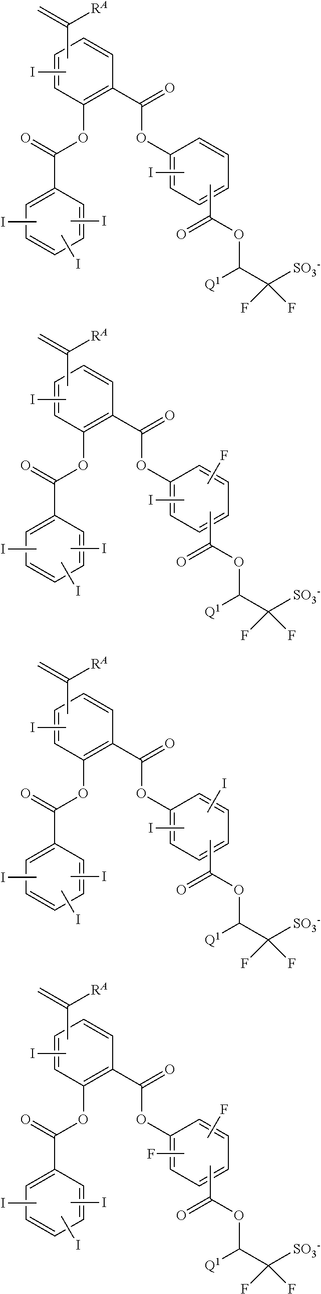

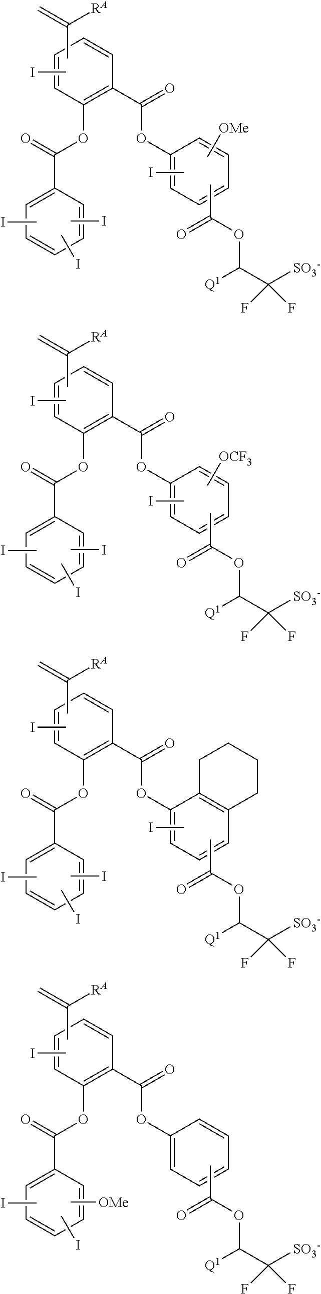

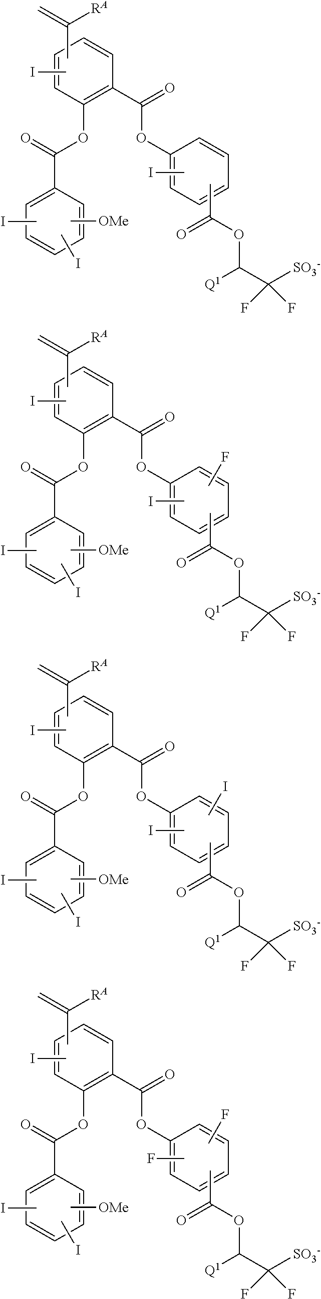

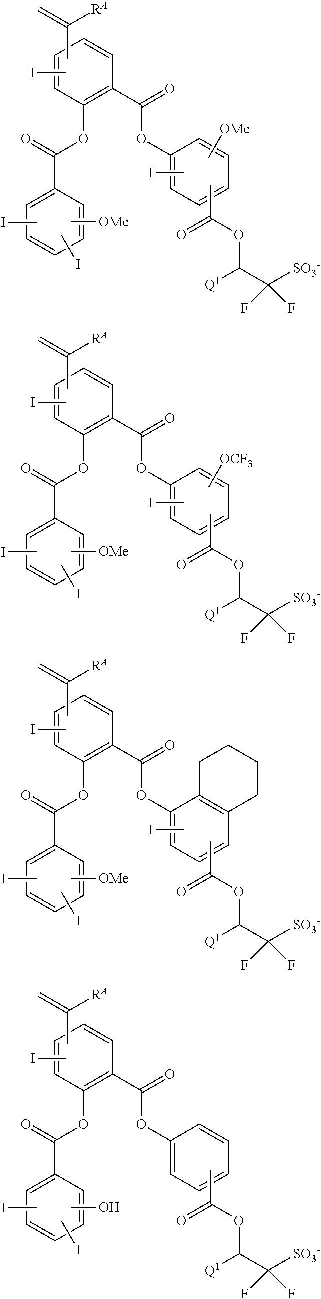

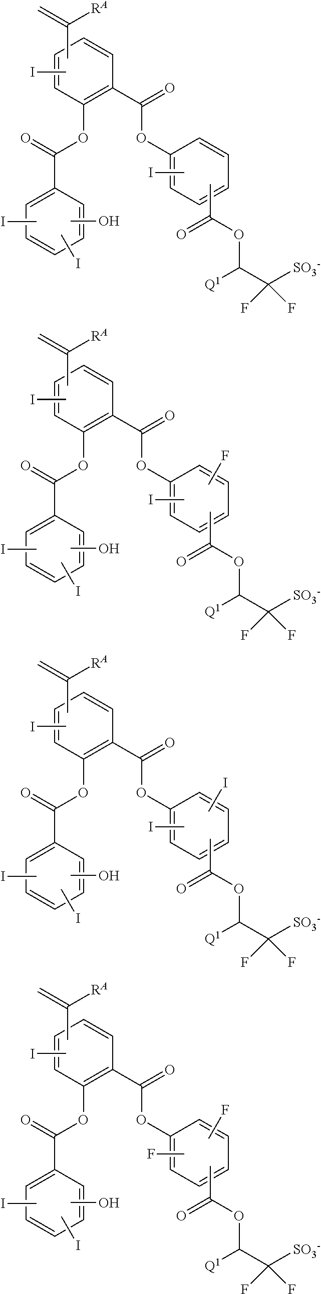

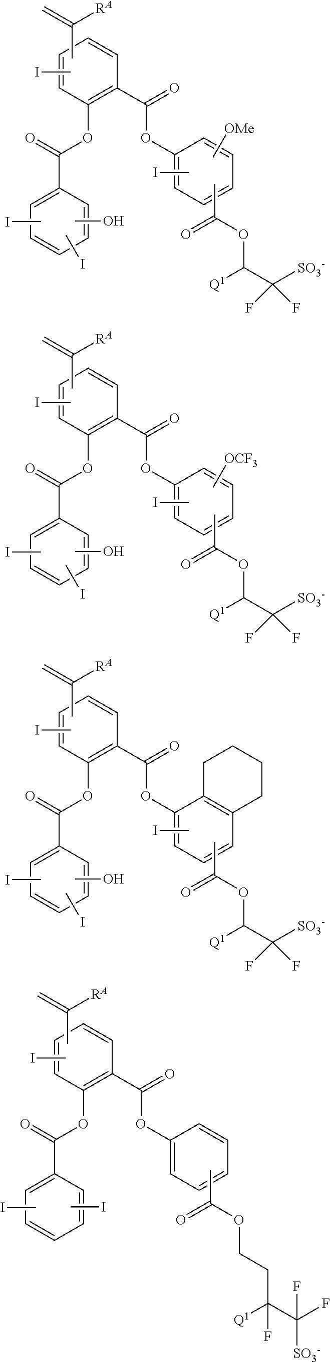

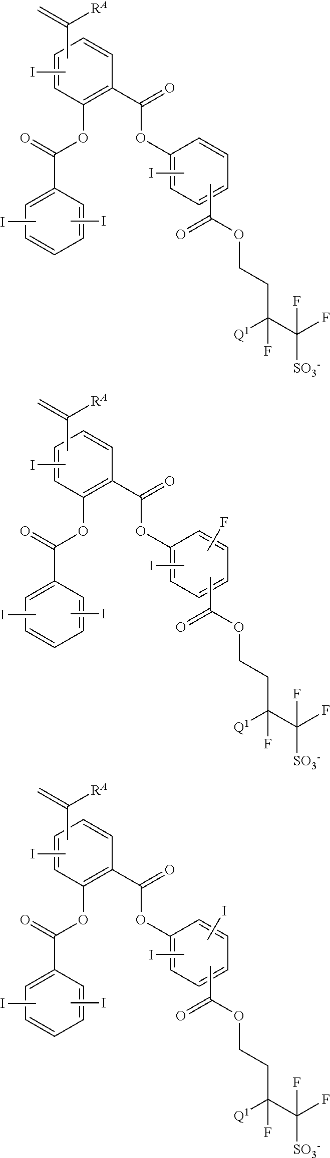

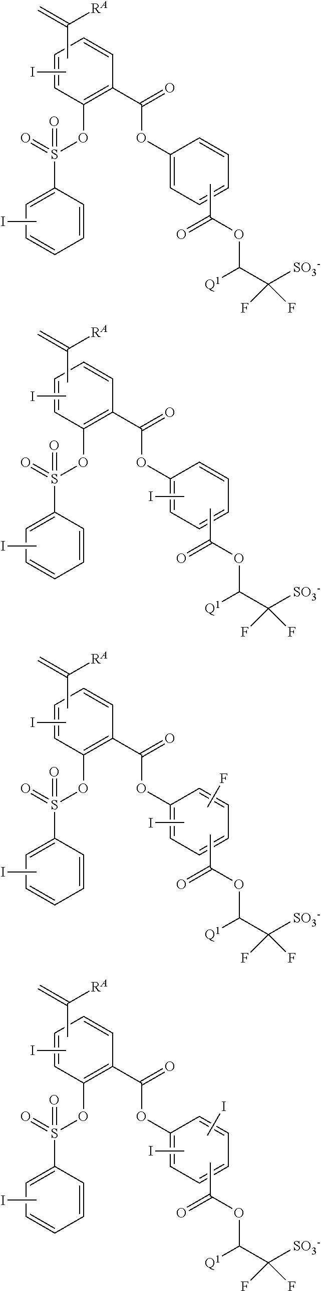

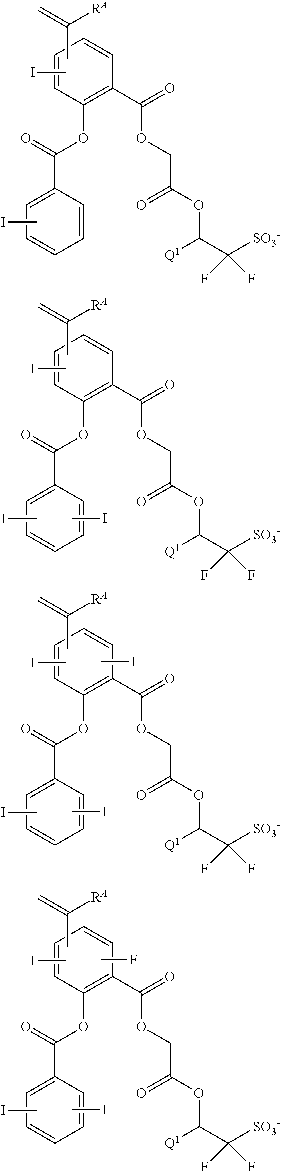

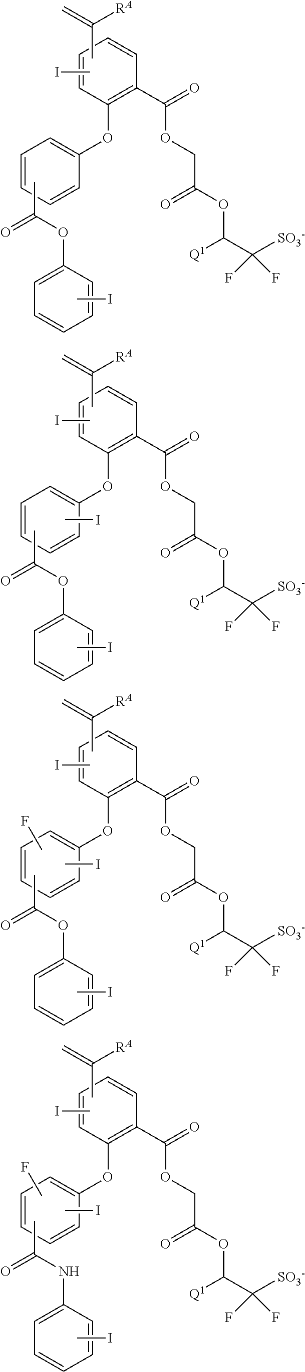

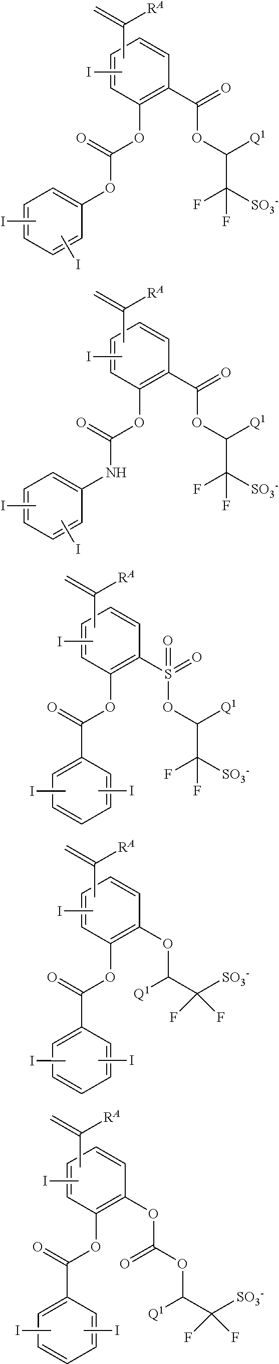

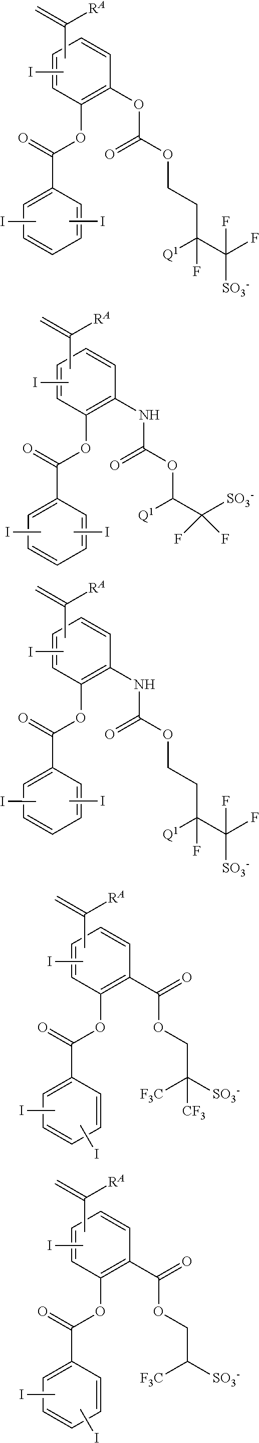

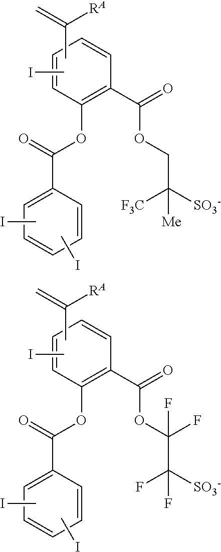

Examples of a specific structure of the sulfonium salt monomer of the invention include arbitrary combinations of the anion with the cation described above.

The sulfonium salt monomer of the invention can be synthesized by a known method. For example, first, a corresponding sulfoxide is reacted with a Grignard reagent in the presence of a halosilicon reagent to synthesize a sulfonium salt containing the sulfonium cation. Subsequently, the synthesized sulfonium salt can be converted into an intended sulfonium salt by being subjected to a salt exchange reaction with a corresponding anion. The salt exchange with the corresponding anion can be easily performed by a known method, and for example, JP-A 2007-145797 can be referred to.

The production method is merely an example, and the method for producing a sulfonium salt of the invention is not limited thereto.

Examples of the structural characteristics of the sulfonium salt monomer of the invention include a fluorosulfonic acid anion structure including a polymerizable group having an aromatic vinyl structure and an iodine atom, and substitution of a —SF₅ group on an aromatic ring of a sulfonium cation. As for an iodine atom and a fluorine atom, an iodine atom is highly absorptive to EUV particularly in EUV lithography with a wavelength of 13.5 nm, and therefore secondary electrons are generated from the iodine atom upon exposure. The fluorine atom is also an element absorptive to EUV although not as much as the iodine atom. In addition, a polymerizable group is included in the anion moiety, and therefore can be copolymerized with the base polymer, and the diffusion of the generated acid can be prevented. In particular, a polymerizable group having a styrene or vinylnaphthalene structure is more robust than a polymerizable group such as a methacrylic acid ester and improves the glass transition temperature (Tg) of the polymer. Owing to the interaction of aromatic rings within or between the base polymers (71-71 stacking effect), the base polymers are arranged in order, and resistance to pattern collapse is exhibited against a developer even when a fine pattern is formed. Also in the etching step after formation of a fine pattern, excellent etching resistance is exhibited owing to the inclusion of an aromatic ring directly connected to the backbone. On the other hand, the —SF₅ group substituted on the aromatic ring of the sulfonium cation is known as a strong electron withdrawing group having high solvent solubility. The iodine atom is an element having relatively low solvent solubility, but sufficient solvent solubility as a sulfonium salt can be ensured due to the effect of this group. In addition, since the group has a strong electron withdrawing property, it is considered that the energy level of LUMO in the frontier orbital theory decreases. Therefore, the secondary electrons generated from the iodine atom in the anion are easily received, the decomposition of the cation is promoted, and the acid is efficiently generated. The sensitivity is increased by these synergistic effects, lowering of resolution due to blur by acid diffusion can be prevented, and it is possible to improve LWR and CDU. Thus, the polymer of the invention is suitable in particular as a material for a chemically amplified positive resist composition.

### Polymer

The polymer of the invention contains a repeat unit derived from the sulfonium salt monomer having the formula (A) (hereinafter also referred to as repeat unit A).

The polymer may contain a repeat unit having the formula (a1) (hereinafter also referred to as repeat unit a1) or a repeat unit having the formula (a2) (hereinafter also referred to as repeat unit a2).

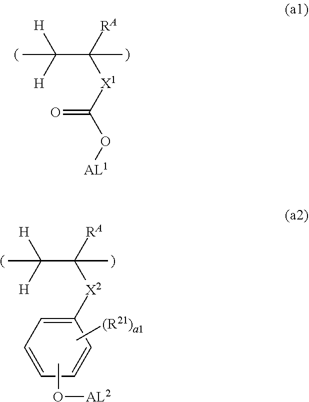

In the formulae (a1) and (a2), R^{A}'s are each independently a hydrogen atom, a fluorine atom, a methyl group, or a trifluoromethyl group.

In the formula (a1), X¹ is a single bond, a phenylene group, a naphthylene group, or *—C(═O)—O—X¹¹—, and the phenylene group or the naphthylene group may be substituted with a hydroxy group, a nitro group, a cyano group, a C₁-C₁₀ saturated hydrocarbyl group which may contain a fluorine atom, a C₁-C₁₀ saturated hydrocarbyloxy group which may contain a fluorine atom, or a halogen atom, X¹¹ is a C₁-C₁₀ saturated hydrocarbylene group, a phenylene group, or a naphthylene group, and the saturated hydrocarbylene group may contain a hydroxy group, an ether bond, an ester bond, or a lactone ring, and * designates a point of attachment to a carbon atom in the backbone.

In the formula (a2), X² is a single bond or *—C(═O)—O—, * designates a point of attachment to a carbon atom in the backbone, R²¹ is a halogen atom, a cyano group, a hydroxy group, a nitro group, a C₁-C₂₀ hydrocarbyl group which may contain a heteroatom, a C₁-C₂₀ hydrocarbyloxy group which may contain a heteroatom, a C₂-C₂₀ hydrocarbylcarbonyl group which may contain a heteroatom, a C₂-C₂₀ hydrocarbylcarbonyloxy group which may contain a heteroatom, or a C₂-C₂₀ hydrocarbyloxycarbonyl group which may contain a heteroatom, a1 is 0, 1, 2, 3, or 4, preferably 0 or 1, and when a1 is 2 or more, a plurality of R²¹'s may be identical or different.

In the formulae (a1) and (a2), AL¹ and AL² are each independently an acid labile group. Specific examples of the acid labile group include those described in JP-A 2013-80033 and JP-A 2013-83821.

Typically, specific examples of the acid labile group include those having the formulae (AL-1) to (AL-3).

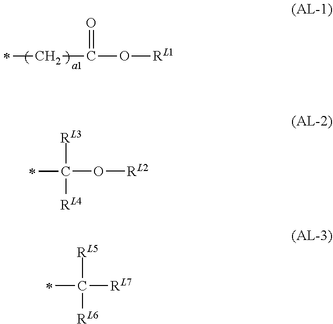

In the formulae, * designates a point of attachment.

In the formulae (AL-1) and (AL-2), R^{L1} and R^{L2} are each independently a C₁-C₄₀ hydrocarbyl group, and may contain a heteroatom such as an oxygen atom, a sulfur atom, a nitrogen atom, or a fluorine atom. The hydrocarbyl group may be saturated or unsaturated and straight, branched, or cyclic. The hydrocarbyl group is preferably a C₁-C₂₀ hydrocarbyl group.

In the formula (AL-1), a2 is an integer of 0 to 10, and is preferably 1, 2, 3, 4, or 5.

In the formula (AL-2), R^{L3} and R^{L4} are each independently a hydrogen atom or a C₁-C₂₀ hydrocarbyl group, and may contain a heteroatom such as an oxygen atom, a sulfur atom, a nitrogen atom, or a fluorine atom. The hydrocarbyl group may be saturated or unsaturated and straight, branched, or cyclic. The hydrocarbyl group is preferably a C₁-C₂₀ hydrocarbyl group. Any two of R^{L2}, R^{L3}, and R^{L4} may bond together to form a C₃-C₂₀ ring with a carbon atom or a carbon atom and an oxygen atom to which they are attached. The ring is preferably a C₄-C₁₆ ring, and is particularly preferably alicyclic.

In the formula (AL-3), R^{L5}, R^{L6}, and R^{L7} are each independently a C₁-C₂₀ hydrocarbyl group, and may contain a heteroatom such as an oxygen atom, a sulfur atom, a nitrogen atom, or a fluorine atom. The hydrocarbyl group may be saturated or unsaturated and straight, branched, or cyclic. The hydrocarbyl group is preferably a C₁-C₂₀ hydrocarbyl group. Any two of R^{L5}, R^{L6}, and R^{L5} may bond together to form a C₃-C₂₀ ring with a carbon atom to which they are attached. The ring is preferably a C₄-C₁₆ ring, and is particularly preferably alicyclic.

Specific examples of the repeat unit a1 include those shown below, but are not limited thereto. In the following formulae, R^{A} and AL¹ are as defined above.

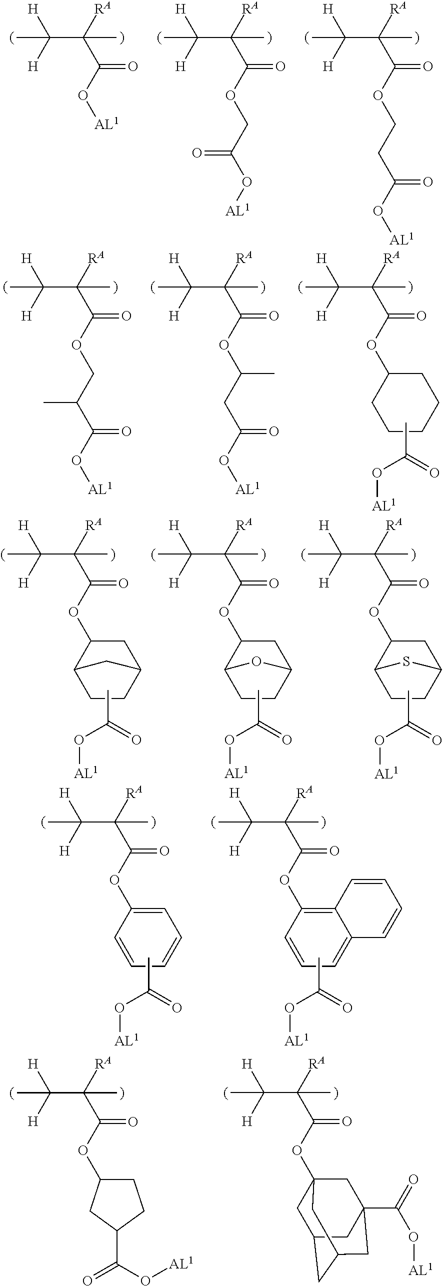

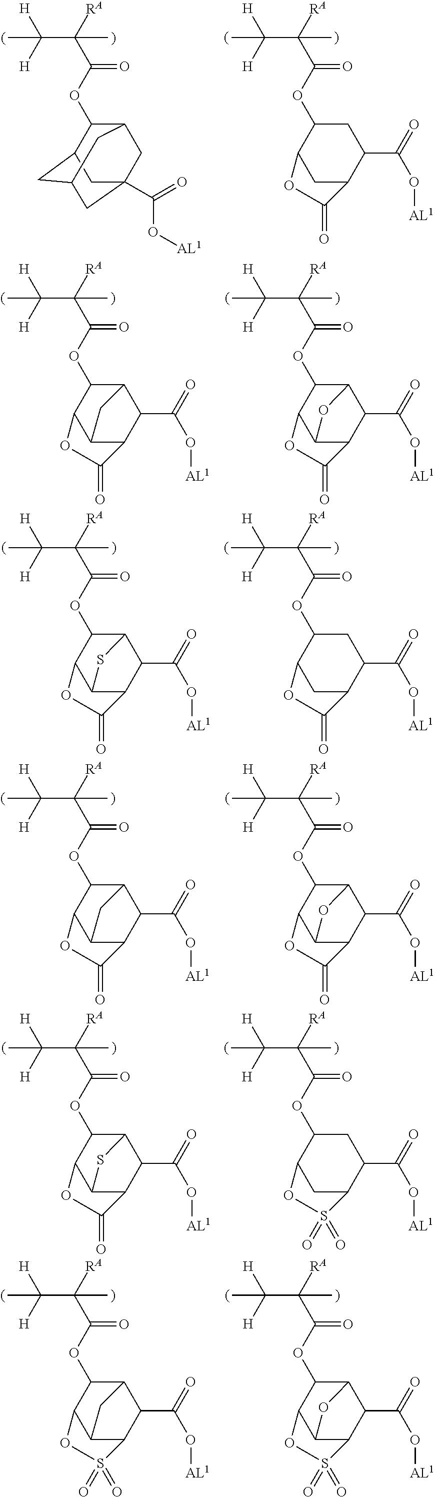

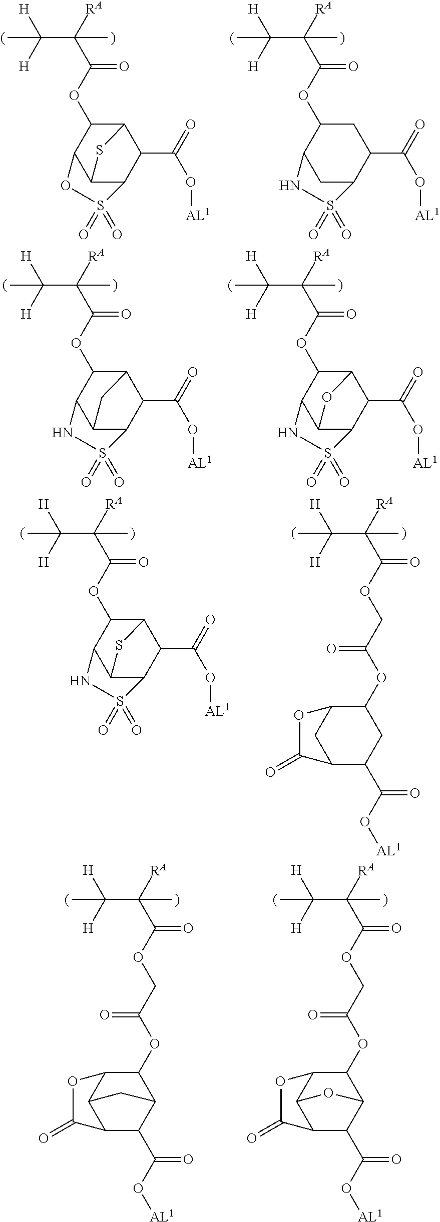

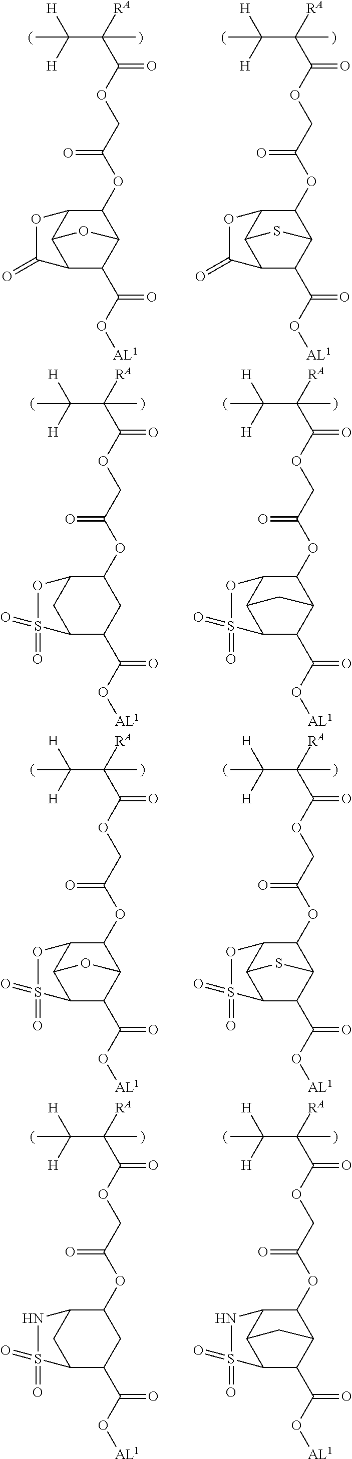

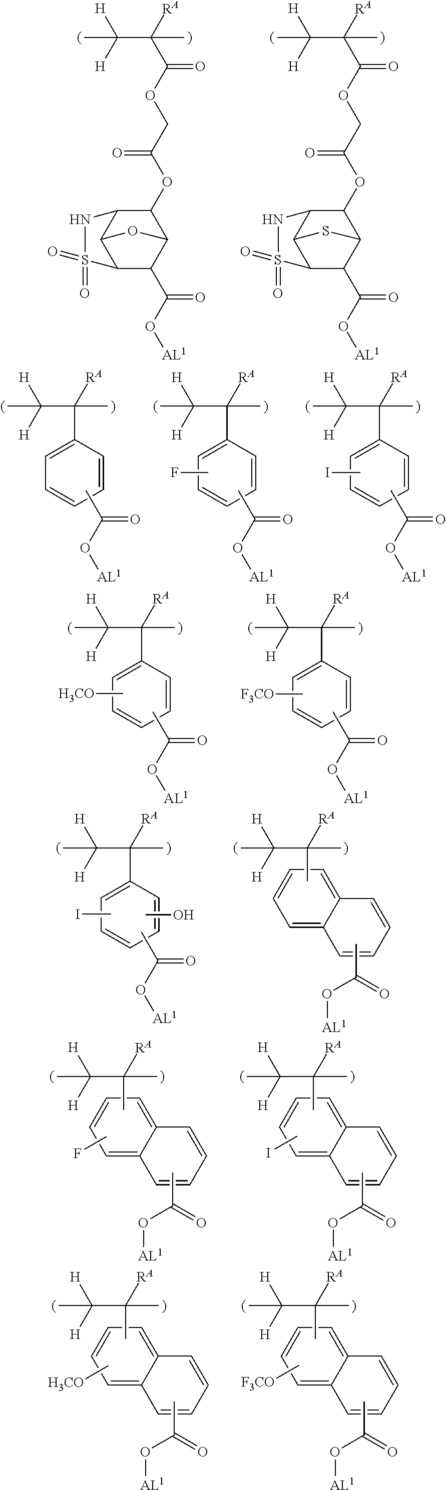

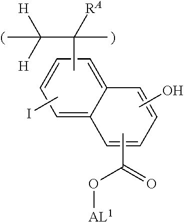

Specific examples of the repeat unit a2 include those shown below, but are not limited thereto. In the following formulae, R^{A} and AL² are as defined above.

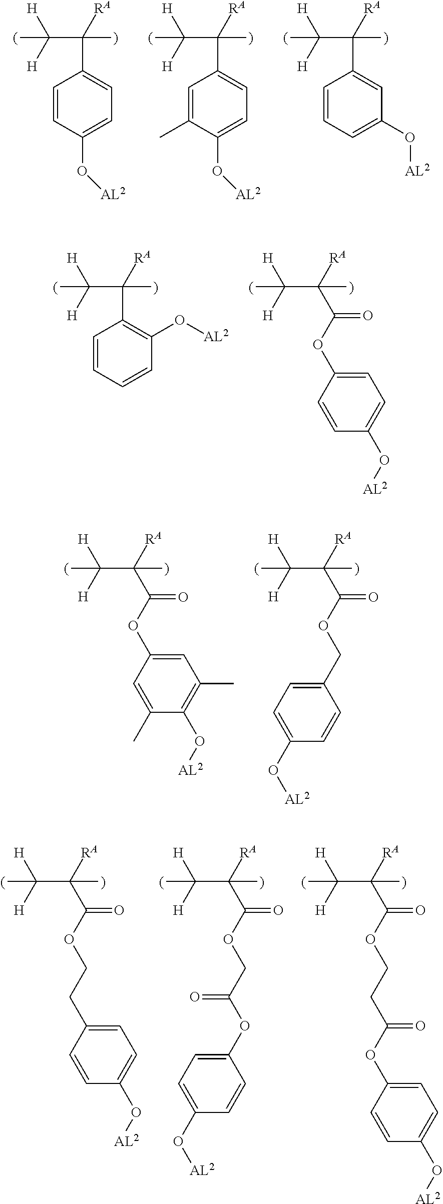

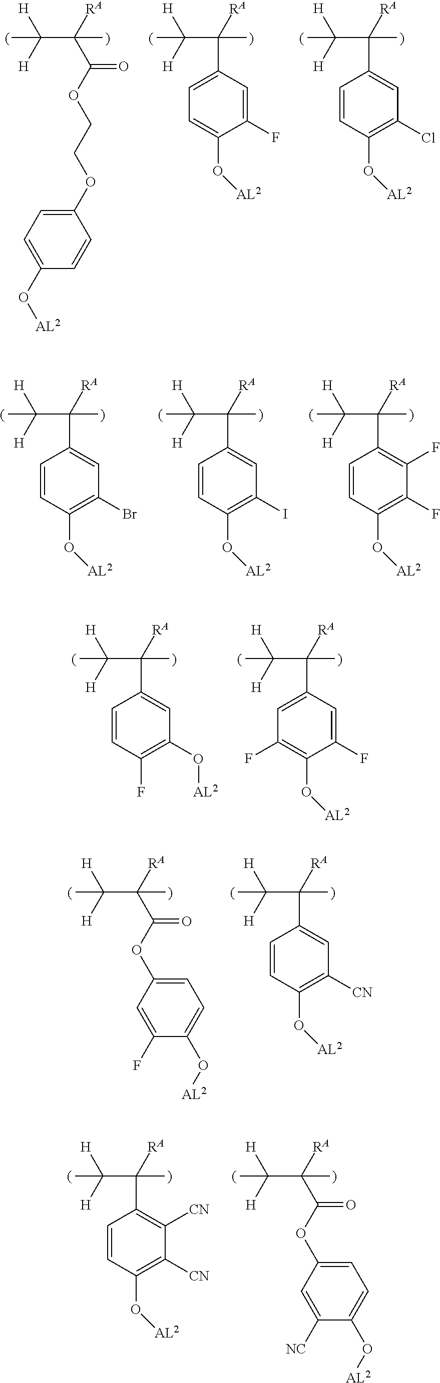

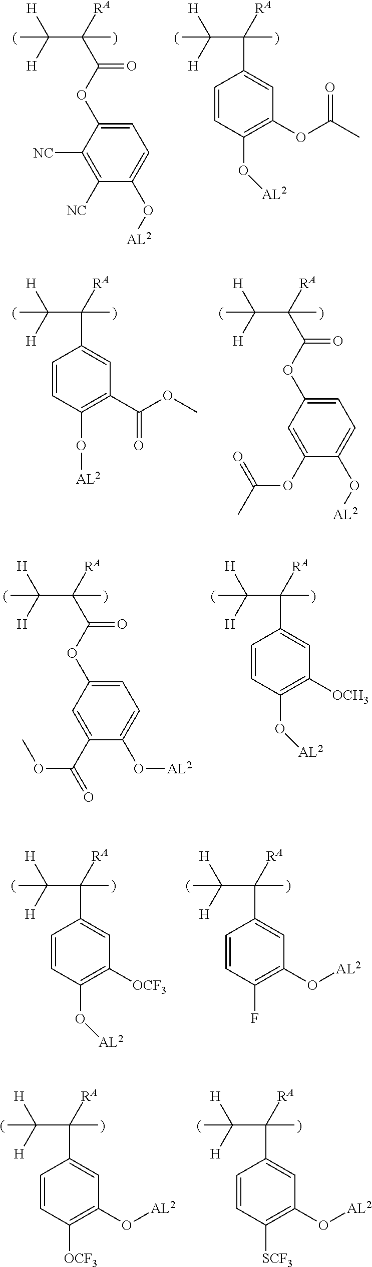

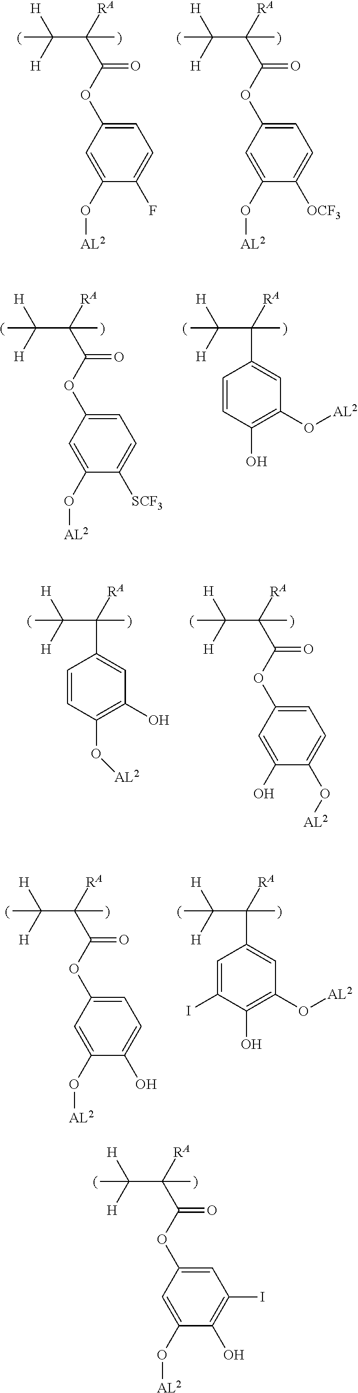

The polymer may further contain a repeat unit having the formula (a3) (hereinafter also referred to as repeat unit a3).

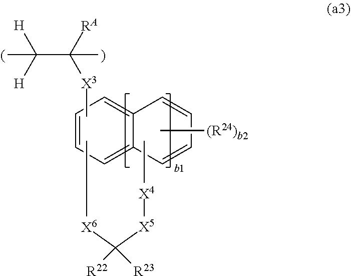

In the formula (a3), b1 is 0 or 1. The relevant structure is a benzene ring when b1 is 0 and a naphthalene ring when b1 is 1, and from the aspect of solvent solubility, a benzene ring when b1 is 0 is preferred. b2 is 0, 1, 2, or 3 when b1 is 0, and is 0, 1, 2, 3, 4, or 5 when b1 is 1. From the aspect of raw material procurement, b2 is preferably 0, 1, 2, or 3, and more preferably 0, 1, or 2.

In the formula (a3), R^{A} is a hydrogen atom, a fluorine atom, a methyl group, or a trifluoromethyl group. Among them, a hydrogen atom and a methyl group are preferred, and a hydrogen atom is more preferred.

In the formula (a3), X³ is a single bond, *—C(═O)—O—, or *—C(═O)—NH—. * designates a point of attachment to a carbon atom in the backbone. Among them, X³ is preferably a single bond or *—C(═O)—O—, and more preferably a single bond.

In the formula (a3), X⁴ is a single bond, a C₁-C₄ aliphatic hydrocarbylene group, a carbonyl group, a sulfonyl group, or a group obtained by combining these groups. Among them, a single bond, a carbonyl group, or a sulfonyl group is preferred from the aspect of raw material procurement, and a single bond or a carbonyl group is more preferred from the viewpoint of a polar group generated after the reaction.

In the formula (a3), X⁵ and X⁶ are each independently an oxygen atom or a sulfur atom. However, X⁴ and X⁶ are attached to an adjacent carbon atom on an aromatic ring. X⁵ and X⁶ may be identical or different, but X⁵ and X⁶ are both preferably an oxygen atom from the aspect of reactivity.

In the formula (a3), R²² and R²³ are each independently a hydrogen atom or a C₁-C₂₀ hydrocarbyl group which may contain a heteroatom. The hydrocarbyl group may be saturated or unsaturated and straight, branched, or cyclic. Specific examples thereof include C₁-C₂₀ alkyl groups such as a methyl group, an ethyl group, a n-propyl group, an isopropyl group, a n-butyl group, an isobutyl group, a sec-butyl group, a tert-butyl group, a n-pentyl group, a n-hexyl group, a n-octyl group, a n-nonyl group, a n-decyl group, an undecyl group, a dodecyl group, a tridecyl group, a tetradecyl group, a pentadecyl group, a heptadecyl group, an octadecyl group, a nonadecyl group, and an icosyl group; C₃-C₂₀ cyclic saturated hydrocarbyl groups such as a cyclopropyl group, a cyclopentyl group, a cyclohexyl group, a cyclopropylmethyl group, a 4-methylcyclohexyl group, a cyclohexylmethyl group, a norbornyl group, and an adamantyl group; C₂-C₂₀ alkenyl groups such as a vinyl group, a 1-propenyl group, a 2-propenyl group, a butenyl group, and a hexenyl group; C₃-C₂₀ cyclic unsaturated hydrocarbyl groups such as a cyclohexenyl group; C₆-C₂₀ aryl groups such as a phenyl group and a naphthyl group; C₇-C₂₀ aralkyl groups such as a benzyl group, a 1-phenylethyl group, and a 2-phenylethyl group; and a group obtained by combining these groups. Some or all hydrogen atoms in the hydrocarbyl group may be substituted with a group containing a heteroatom such as an oxygen atom, a sulfur atom, a nitrogen atom, or a halogen atom, and some —CH₂— in the hydrocarbyl group may be substituted with a group containing a heteroatom such as an oxygen atom, a sulfur atom, or a nitrogen atom, so that the hydrocarbyl group may contain a hydroxy group, a cyano group, a fluorine atom, a chlorine atom, a bromine atom, an iodine atom, a carbonyl group, an ether bond, an ester bond, a sulfonate ester bond, a carbonate bond, a lactone ring, a sultone ring, a carboxylic anhydride (—C(═O)—O—C(═O)—), a haloalkyl group, or the like.

R²² and R²³ may bond together to form a ring with a carbon atom to which they are attached. Specific examples of the ring formed at this time include a cyclopropane ring, a cyclobutane ring, a cyclopentane ring, a cyclohexane ring, a norbornane ring, and an adamantane ring. Some or all hydrogen atoms in the ring may be substituted with a group containing a heteroatom such as an oxygen atom, a sulfur atom, a nitrogen atom, or a halogen atom, and some —CH₂— in the ring may be substituted with a group containing a heteroatom such as an oxygen atom, a sulfur atom, or a nitrogen atom, so that the ring may contain a hydroxy group, a fluorine atom, a chlorine atom, a bromine atom, an iodine atom, a cyano group, a carbonyl group, an ether bond, an ester bond, a sulfonate ester bond, a carbonate bond, a lactone ring, a sultone ring, a carboxylic anhydride (—C(═O)—O—C(═O)—), a haloalkyl group, or the like.

In the formula (a3), R²⁴ is a halogen atom, a hydroxy group, a cyano group, a nitro group, a C₁-C₂₀ hydrocarbyl group which may contain a heteroatom, a C₁-C₂₀ hydrocarbyloxy group which may contain a heteroatom, a C₂-C₂₀ hydrocarbyloxycarbonyl group which may contain a heteroatom, a C₁-C₂₀ hydrocarbylthio group which may contain a heteroatom, or —N(R^{24A})(R^{24B}). R^{24A} and R^{24B} are each independently a hydrogen atom or a C₁-C₆ hydrocarbyl group. The halogen atom is preferably a fluorine atom, a chlorine atom, a bromine atom, or an iodine atom, and more preferably a fluorine atom or an iodine atom. The hydrocarbyl group and the hydrocarbyl moiety of the hydrocarbyloxy group, the hydrocarbyloxycarbonyl group, and the hydrocarbylthio group may be saturated or unsaturated and straight, branched, or cyclic. Specific examples thereof are as exemplified above for the hydrocarbyl group of R²² and R²³. Some or all hydrogen atoms in the hydrocarbyl group may be substituted with a group containing a heteroatom such as an oxygen atom, a sulfur atom, a nitrogen atom, or a halogen atom, and some —CH₂— in the hydrocarbyl group may be substituted with a group containing a heteroatom such as an oxygen atom, a sulfur atom, or a nitrogen atom, so that the hydrocarbyl group may contain a hydroxy group, a cyano group, a fluorine atom, a chlorine atom, a bromine atom, an iodine atom, a carbonyl group, an ether bond, an ester bond, a sulfonate ester bond, a carbonate bond, a lactone ring, a sultone ring, a carboxylic anhydride (—C(═O)—O—C(═O)—), a haloalkyl group, or the like. When b2 is 2 or more, a plurality of R²⁴'s may be identical or different.

Further, when b2 is 2 or more, a plurality of R²⁴'s may bond together to form a ring with a carbon atom on an aromatic ring to which they are attached. Specific examples of the ring formed at this time include a cyclopropane ring, a cyclobutane ring, a cyclopentane ring, a cyclohexane ring, a norbornane ring, and an adamantane ring. Some or all hydrogen atoms in the ring may be substituted with a group containing a heteroatom such as an oxygen atom, a sulfur atom, a nitrogen atom, or a halogen atom, and some —CH₂— in the ring may be substituted with a group containing a heteroatom such as an oxygen atom, a sulfur atom, or a nitrogen atom, so that the ring may contain a hydroxy group, a fluorine atom, a chlorine atom, a bromine atom, an iodine atom, a cyano group, a carbonyl group, an ether bond, an ester bond, a sulfonate ester bond, a carbonate bond, a lactone ring, a sultone ring, a carboxylic anhydride (—C(═O)—O—C(═O)—), a haloalkyl group, or the like.

Specific examples of the repeat unit a3 include those shown below, but are not limited thereto. In the following formulae, R^{A} is as defined above, and Me is a methyl group. The positions of attachment of substituents on the aromatic ring are interchangeable.

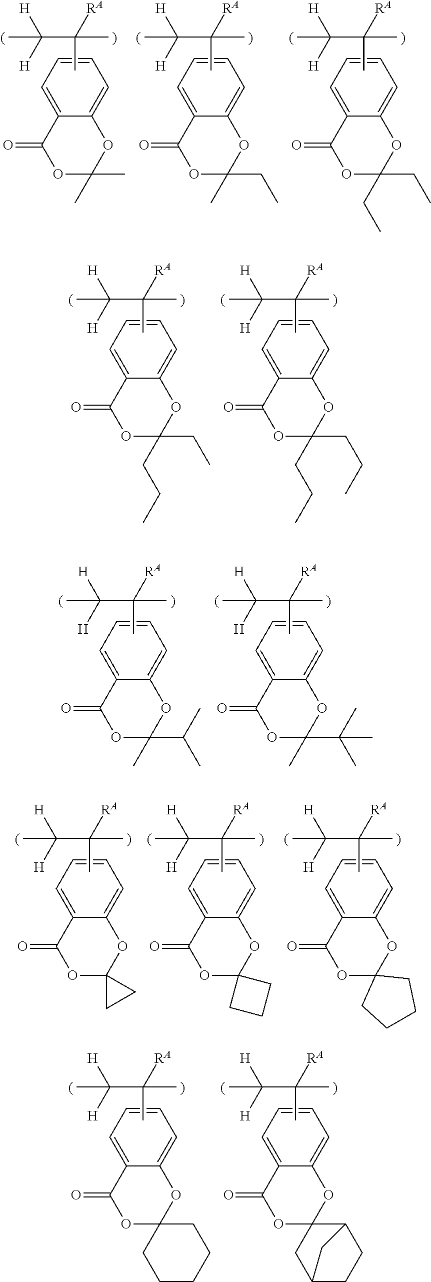

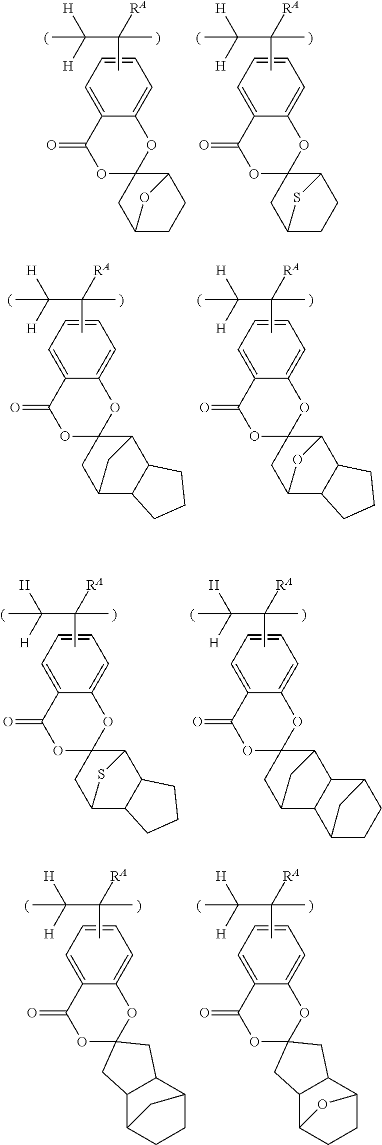

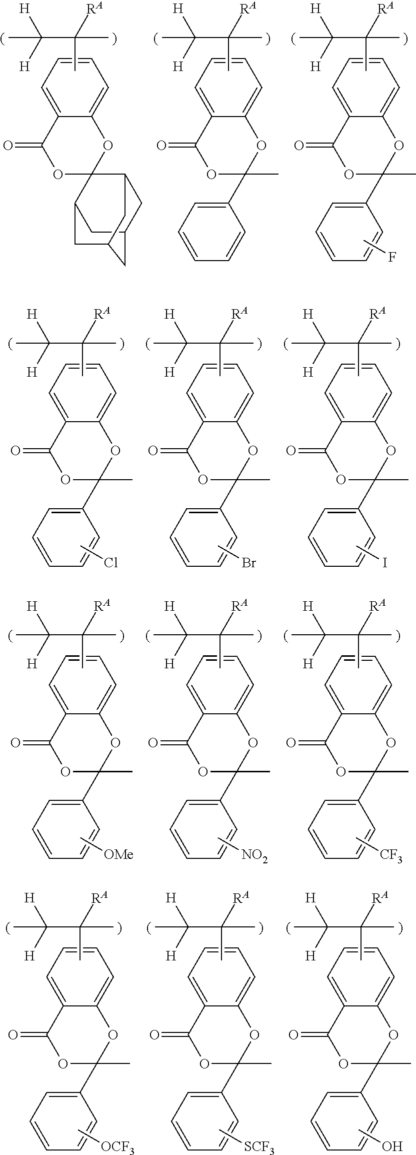

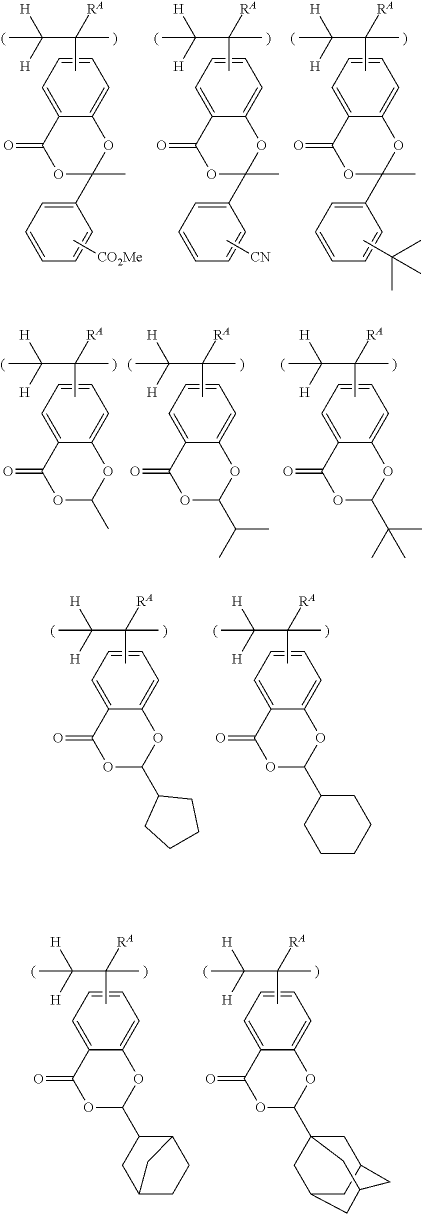

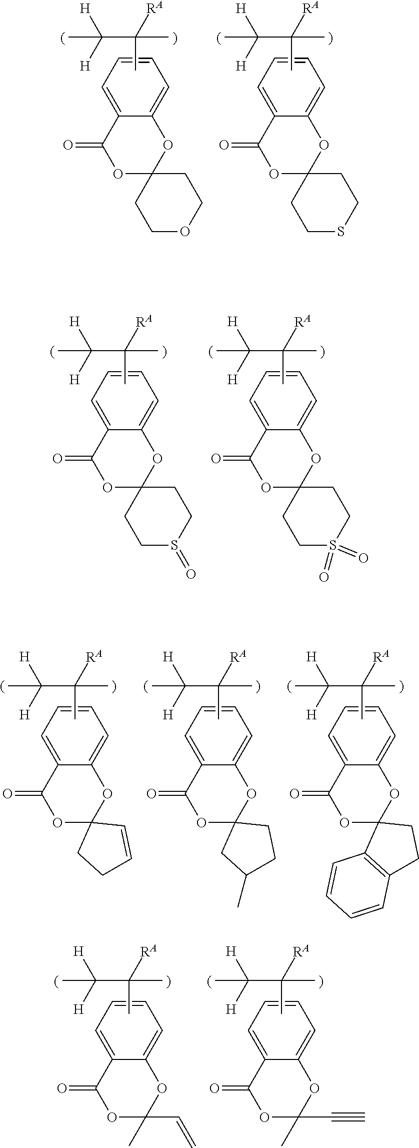

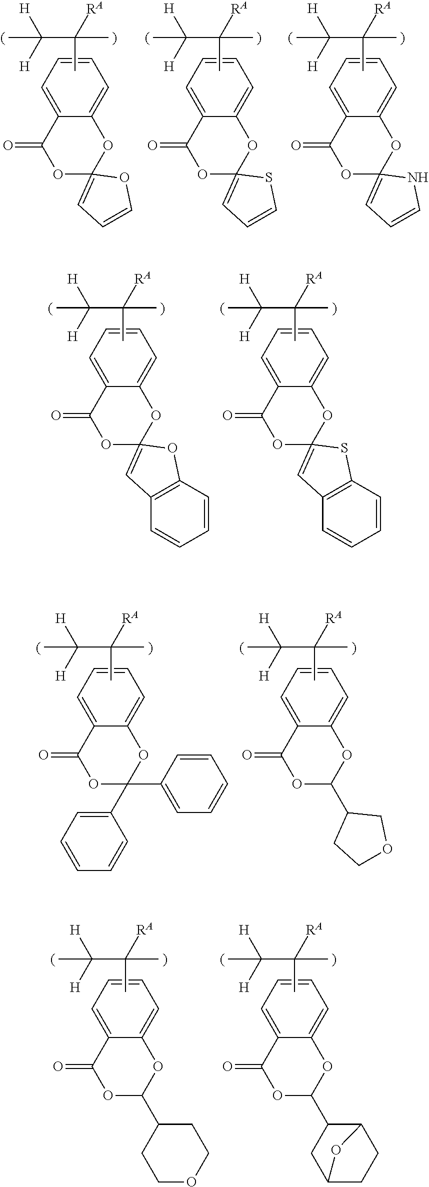

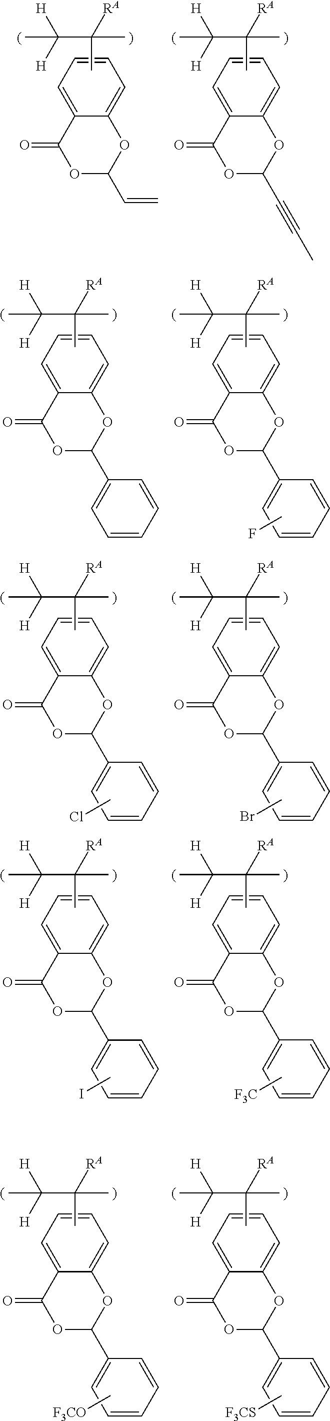

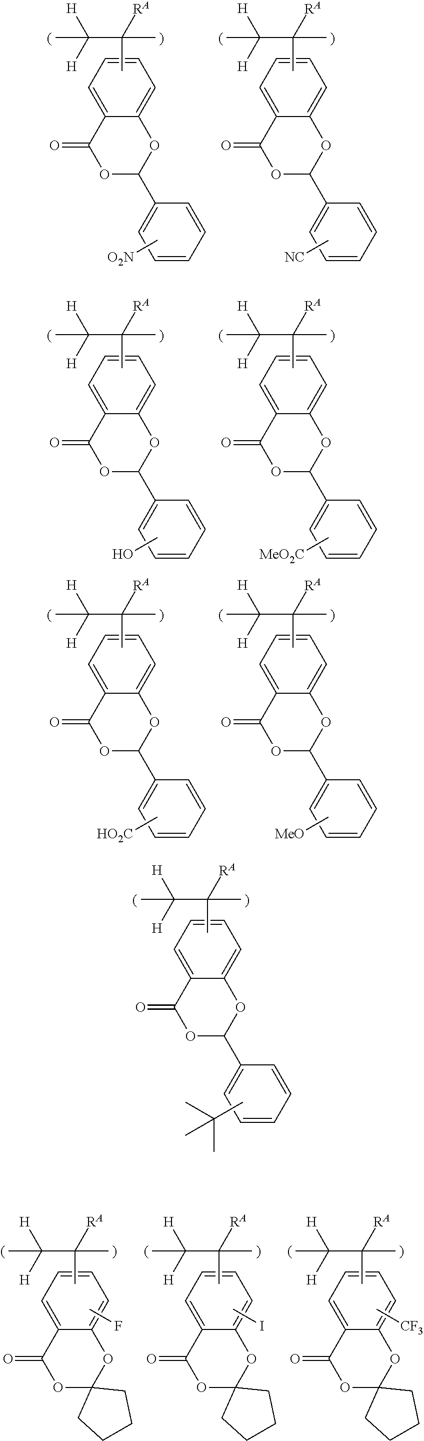

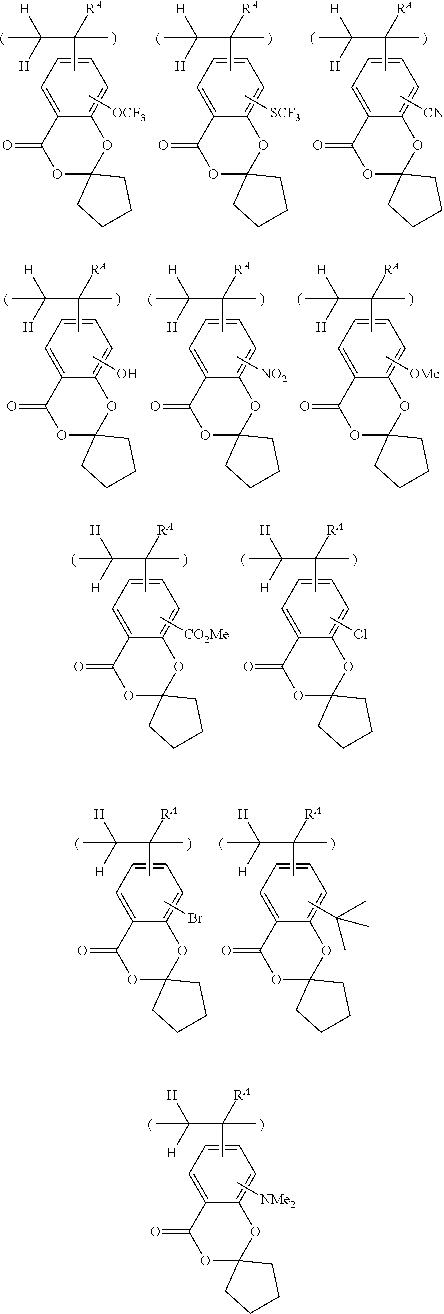

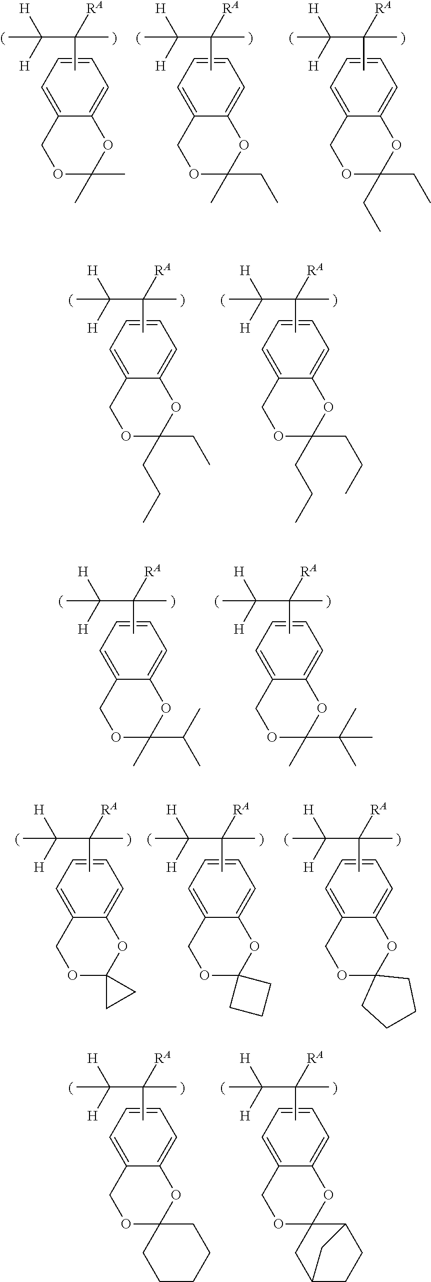

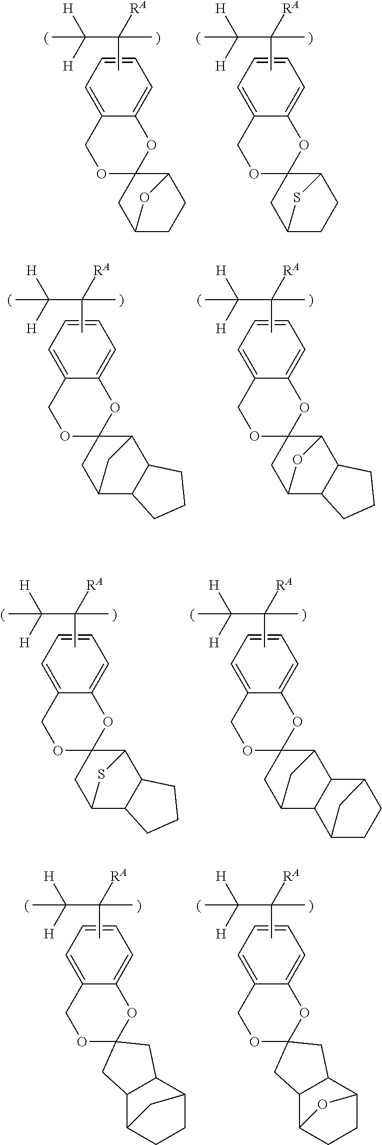

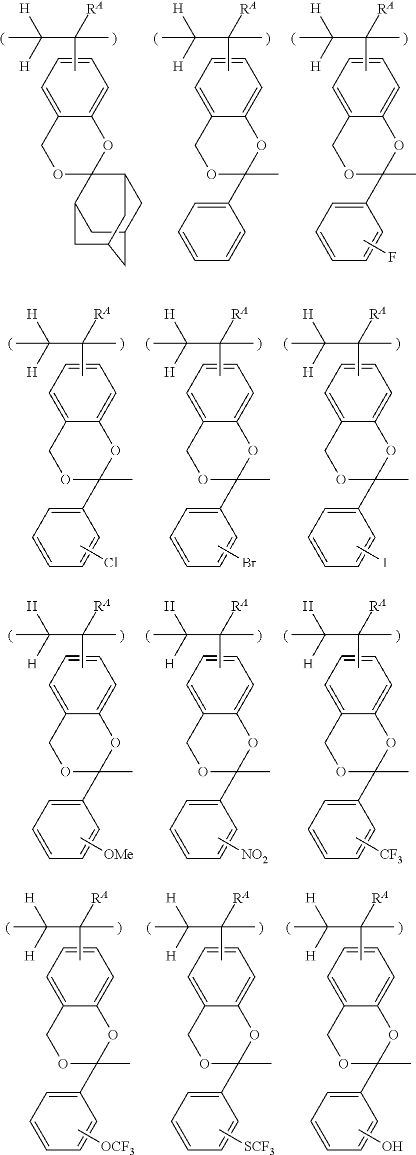

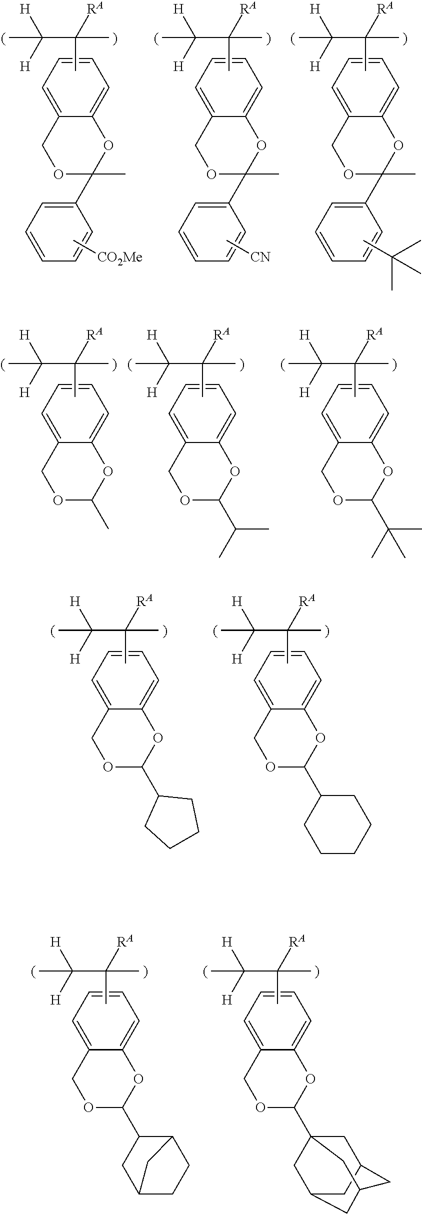

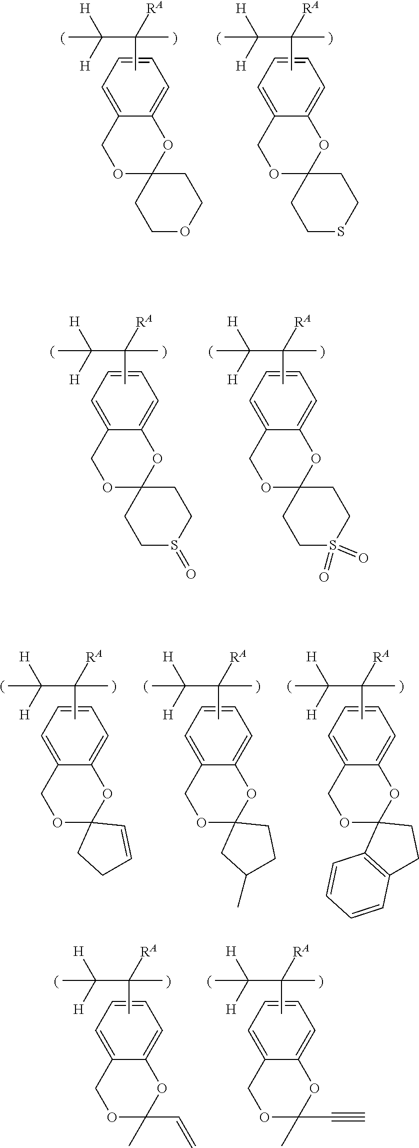

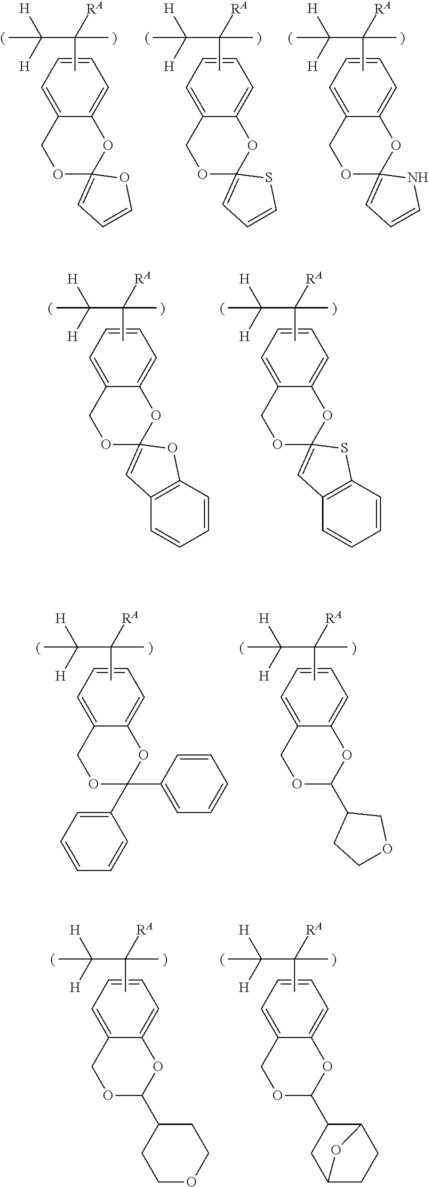

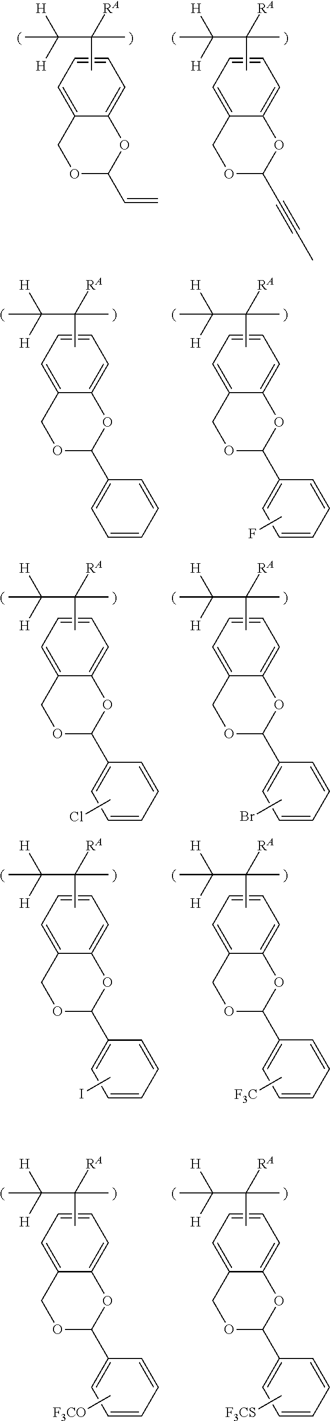

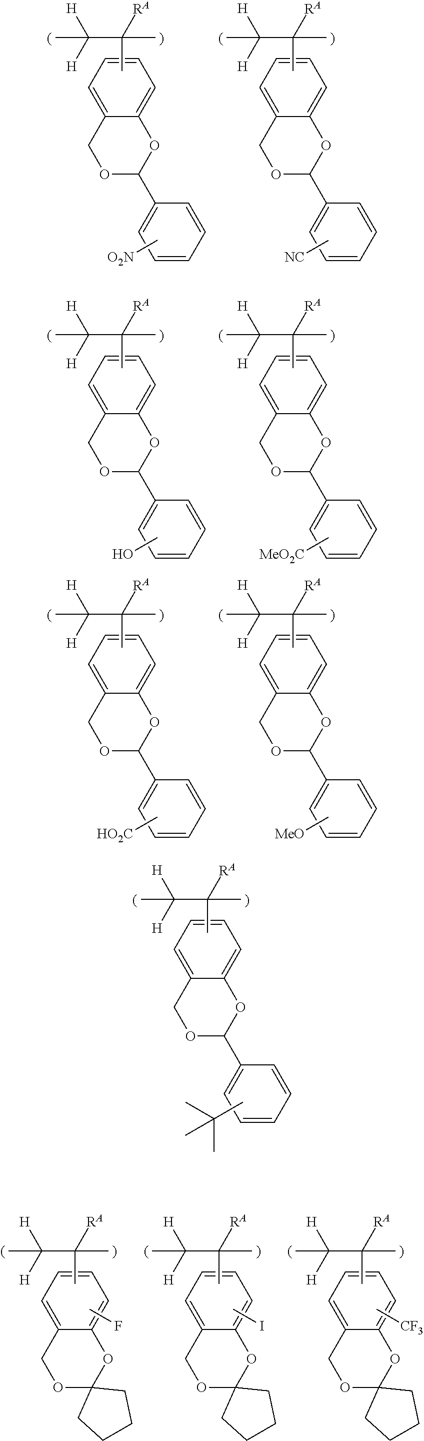

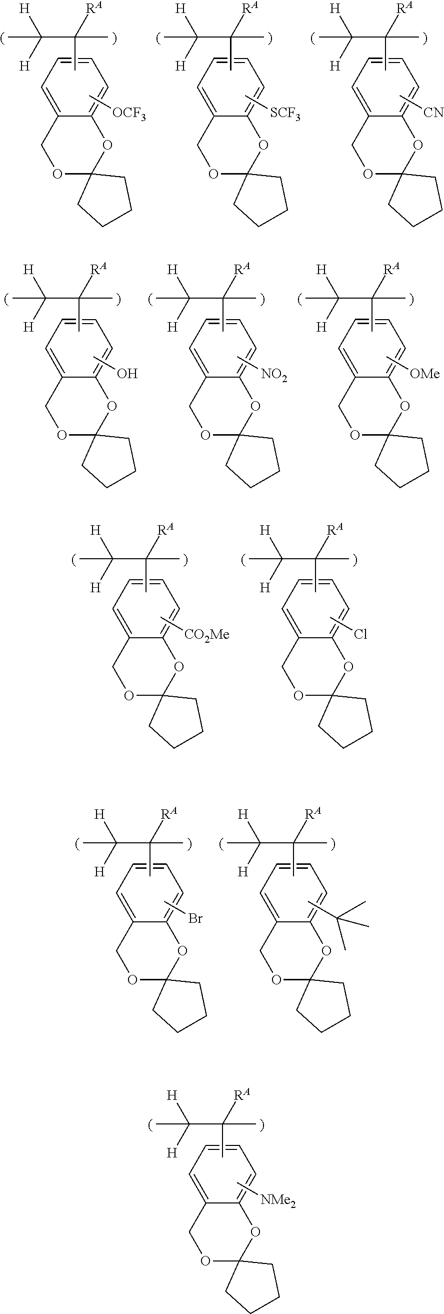

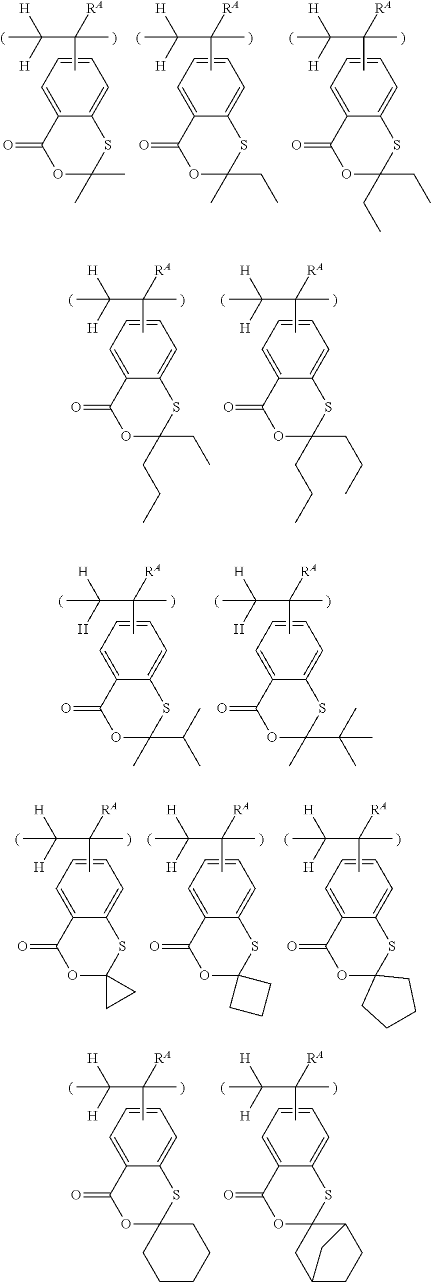

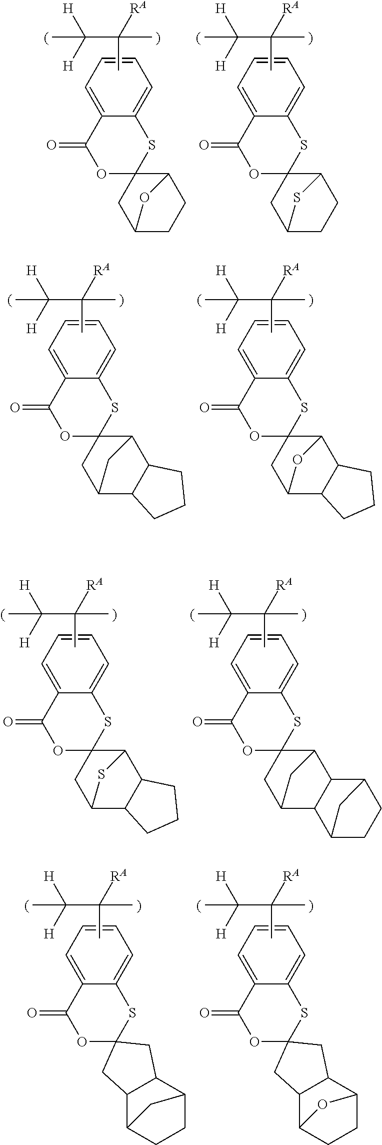

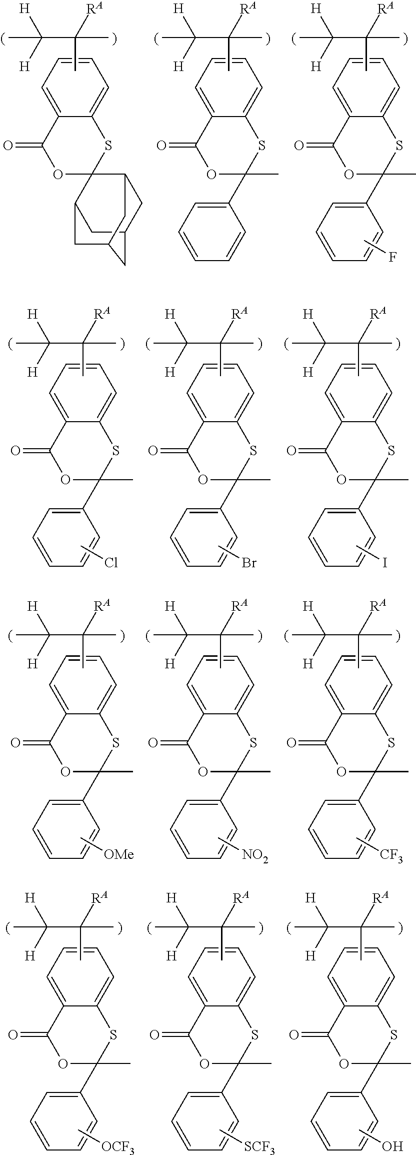

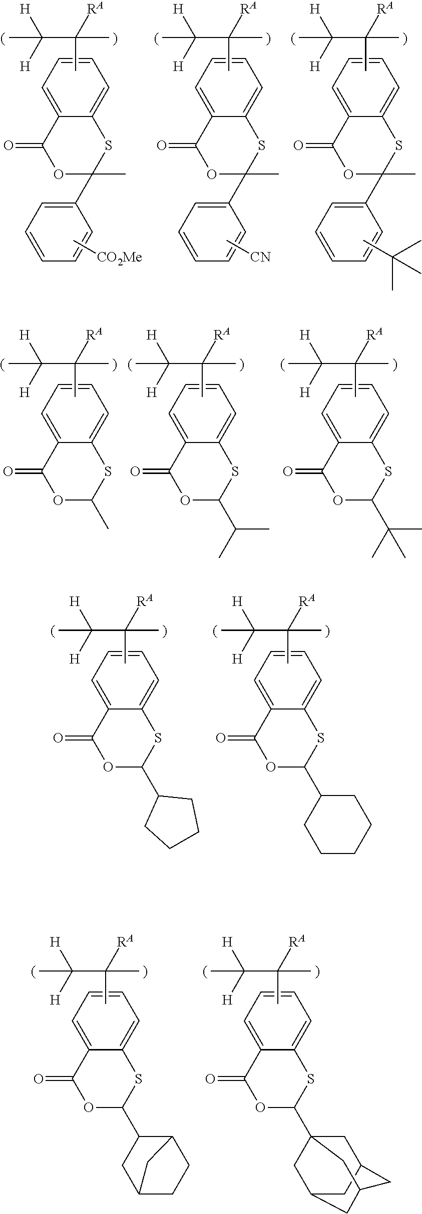

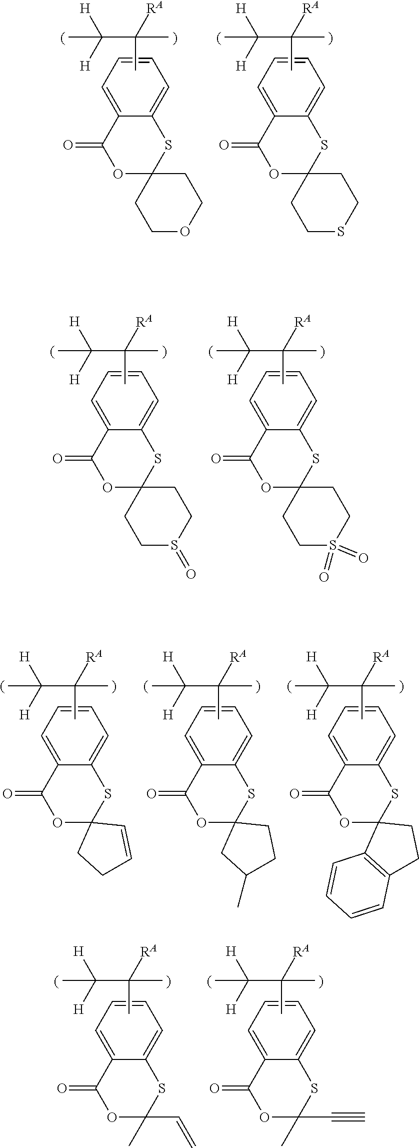

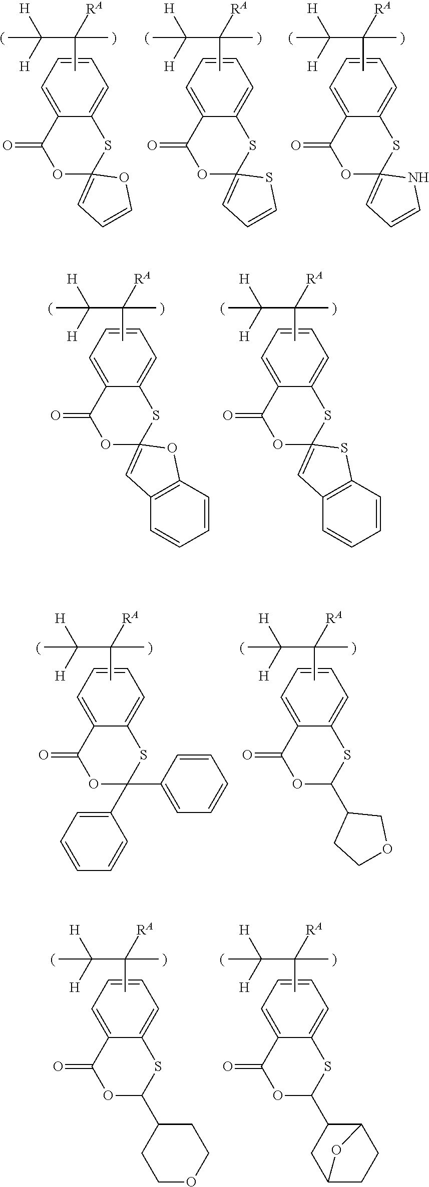

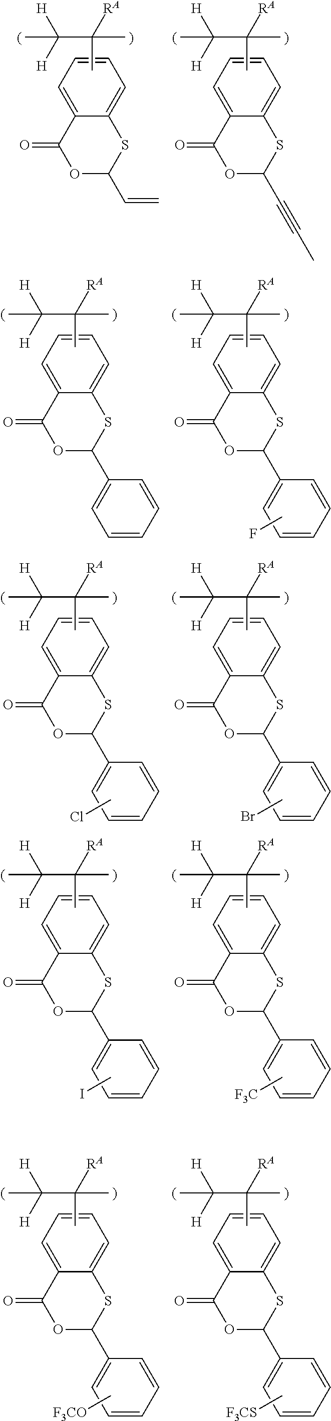

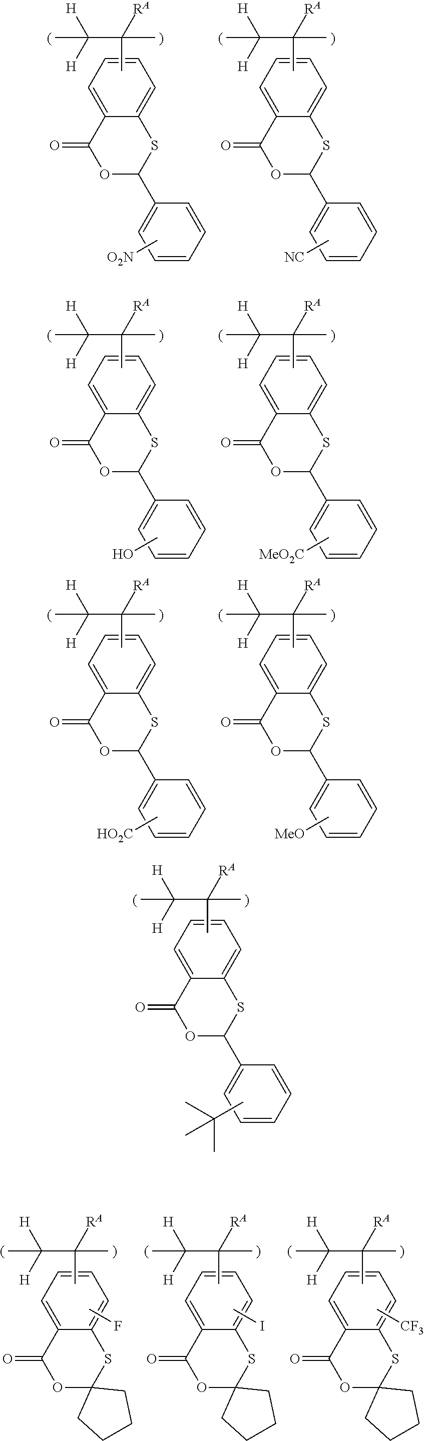

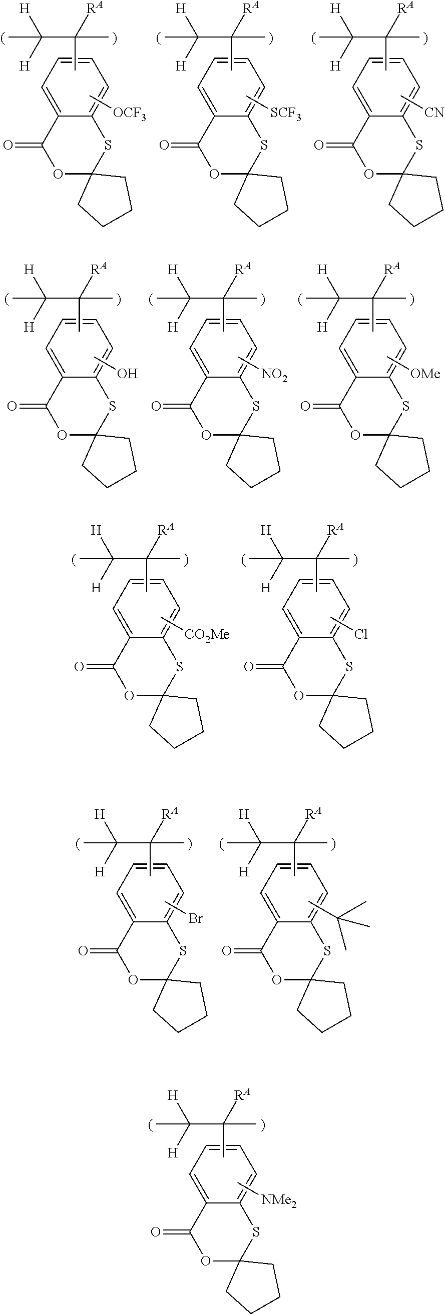

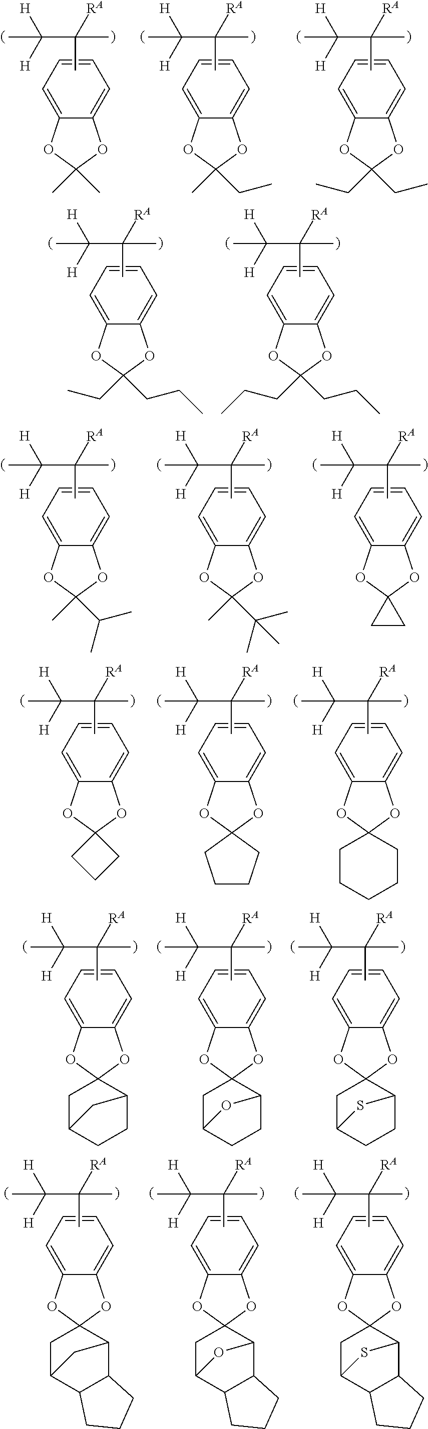

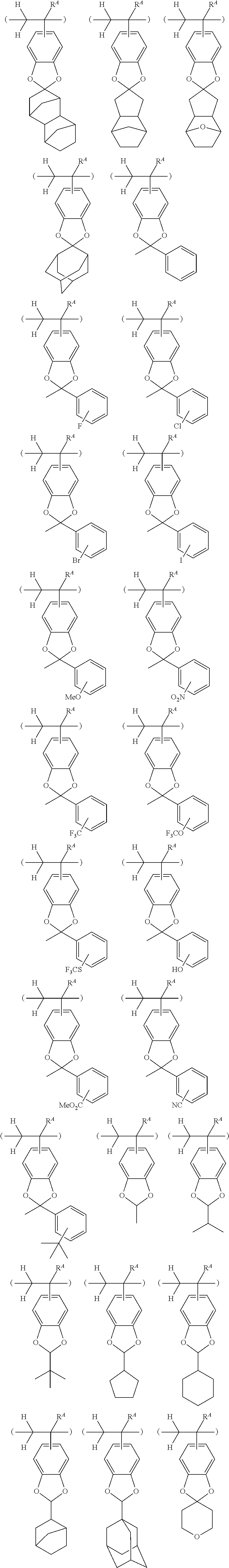

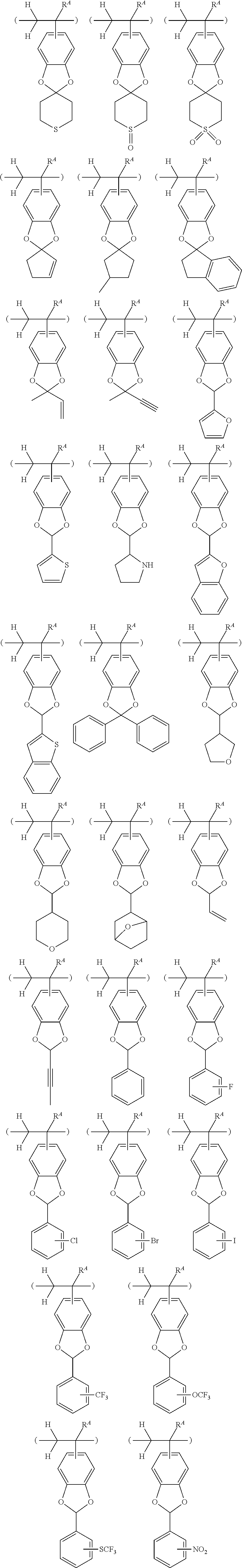

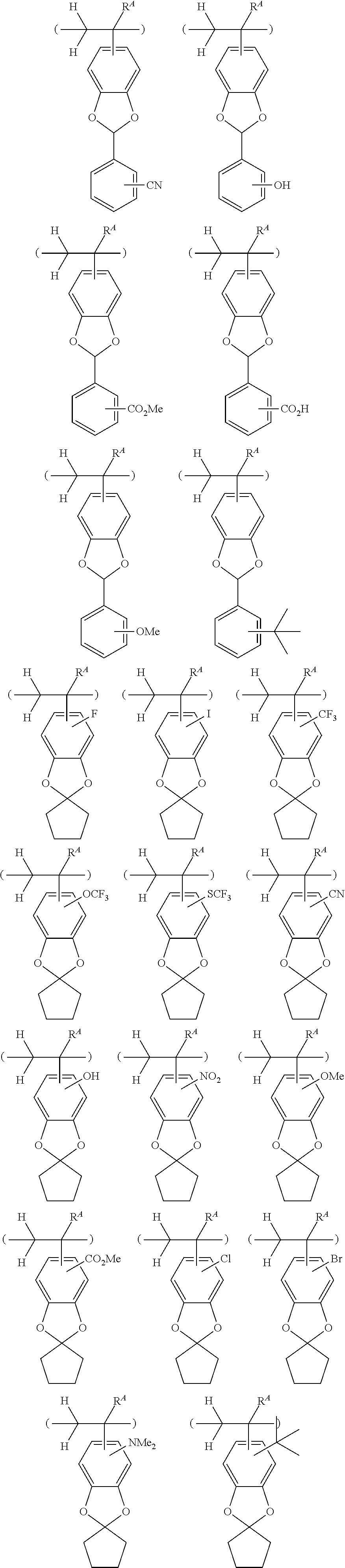

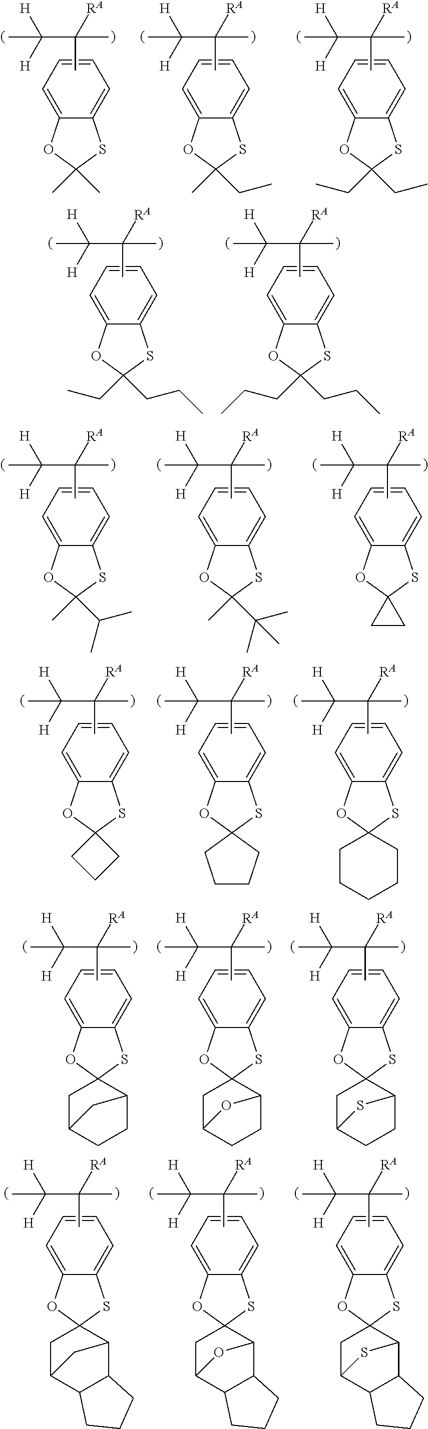

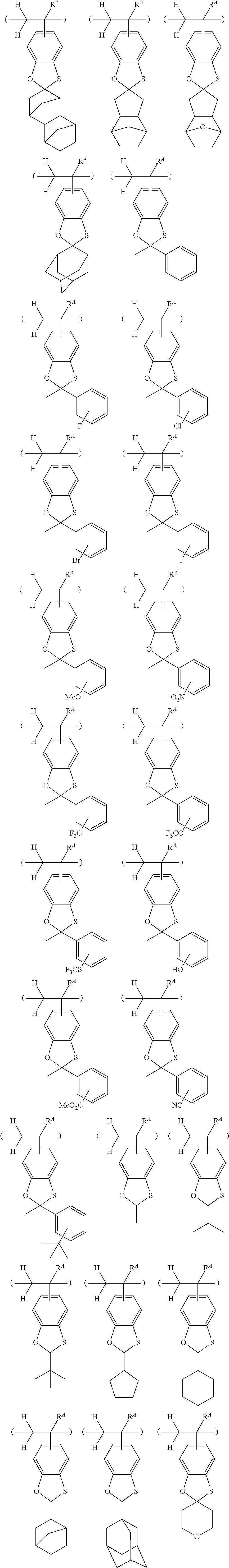

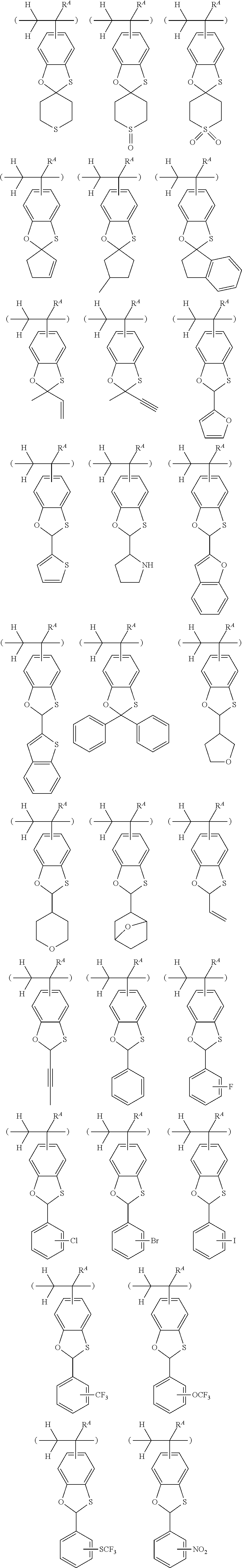

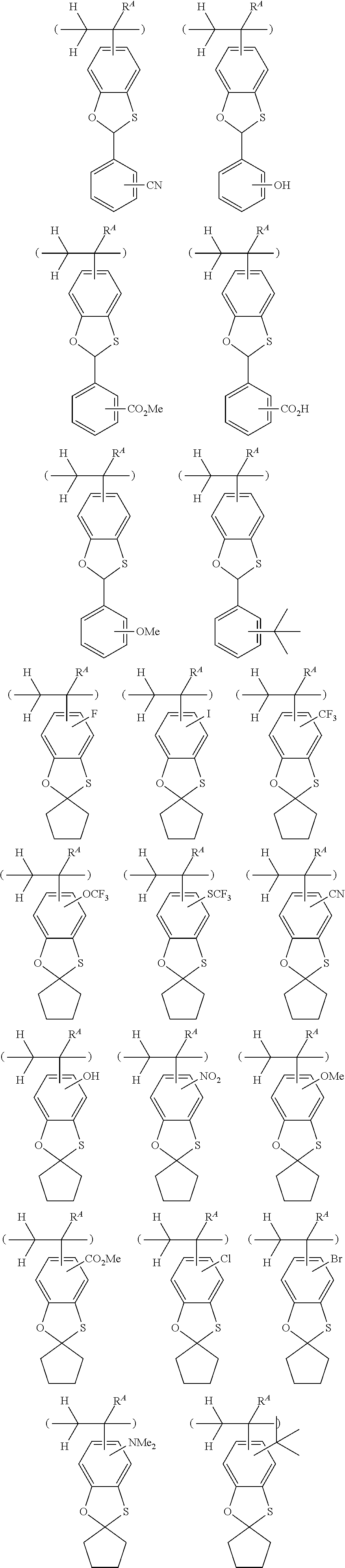

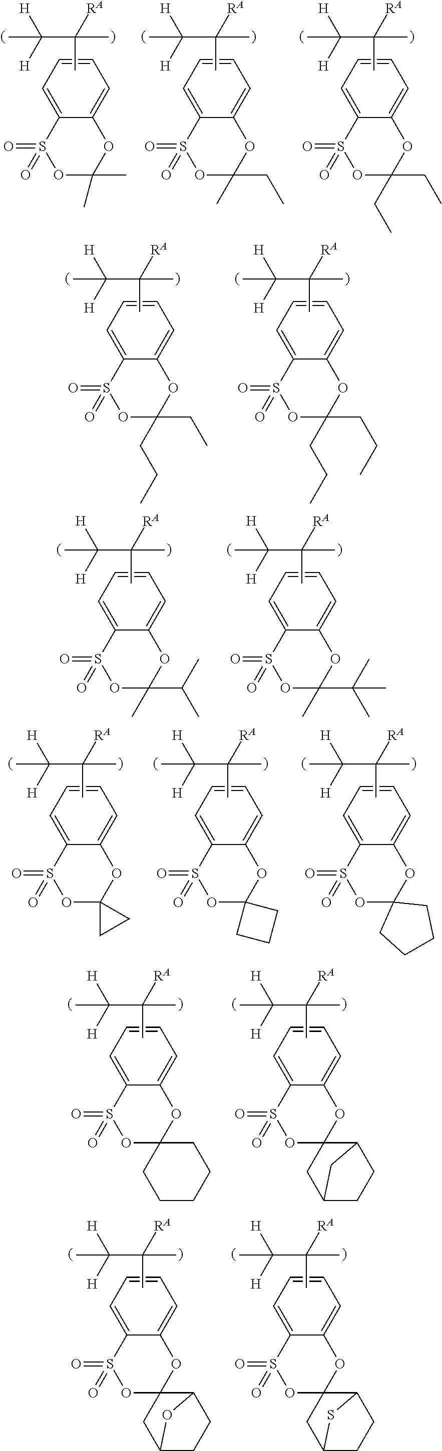

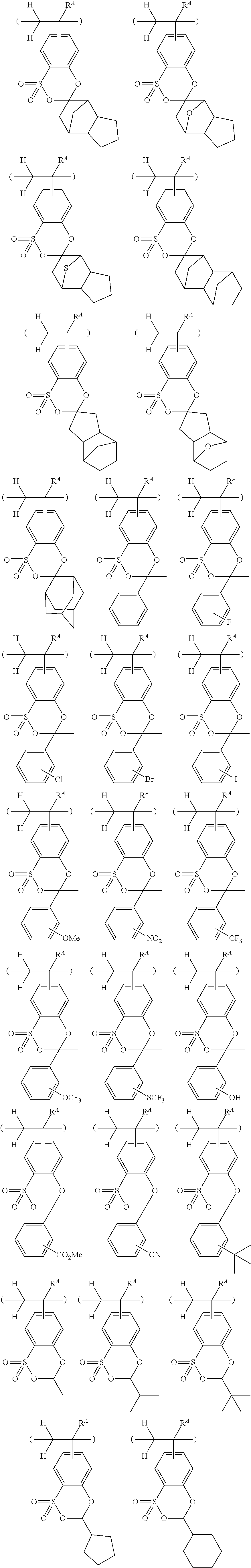

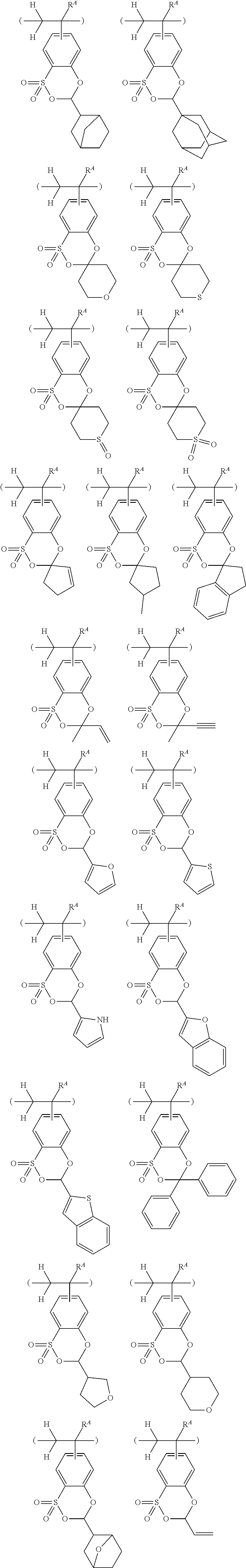

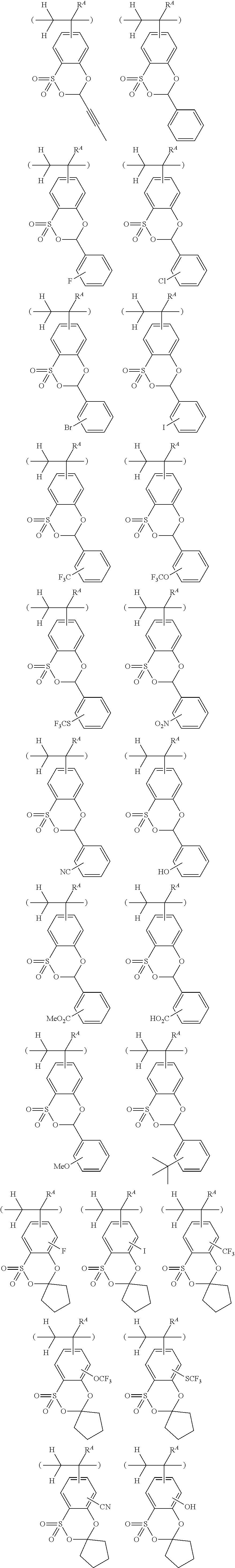

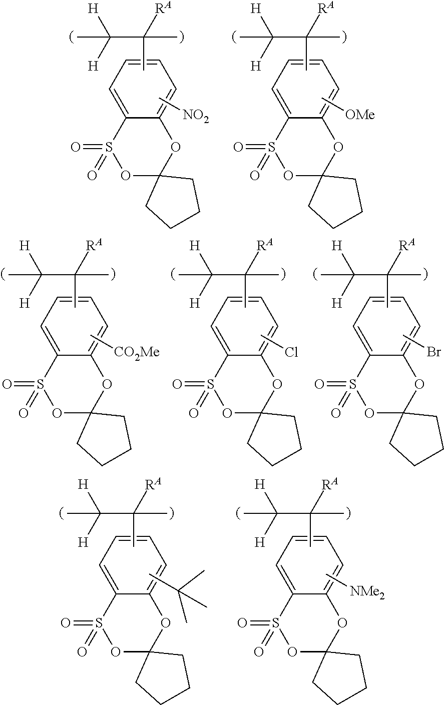

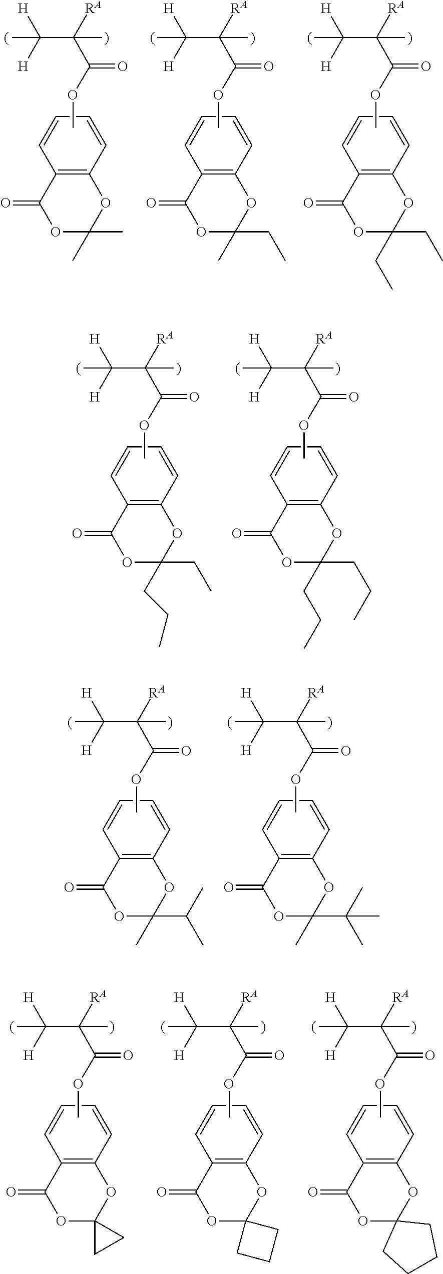

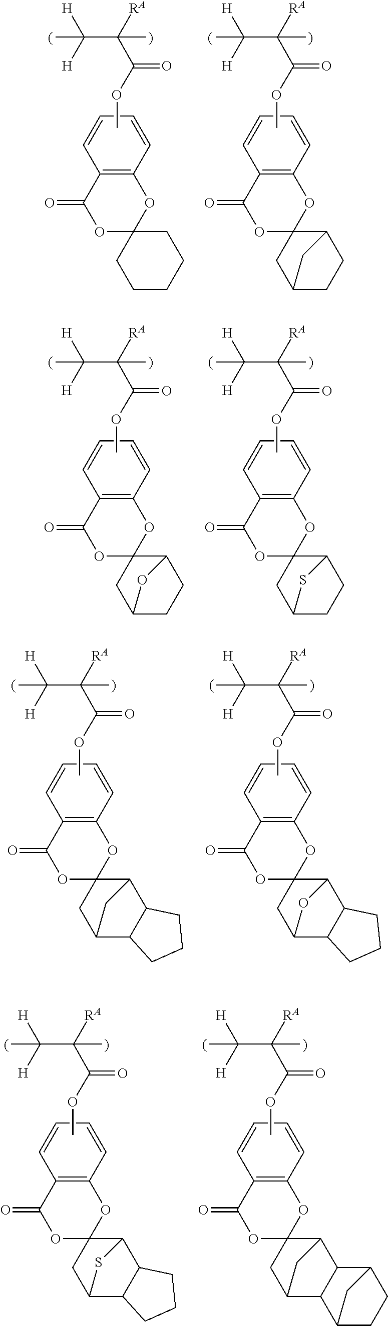

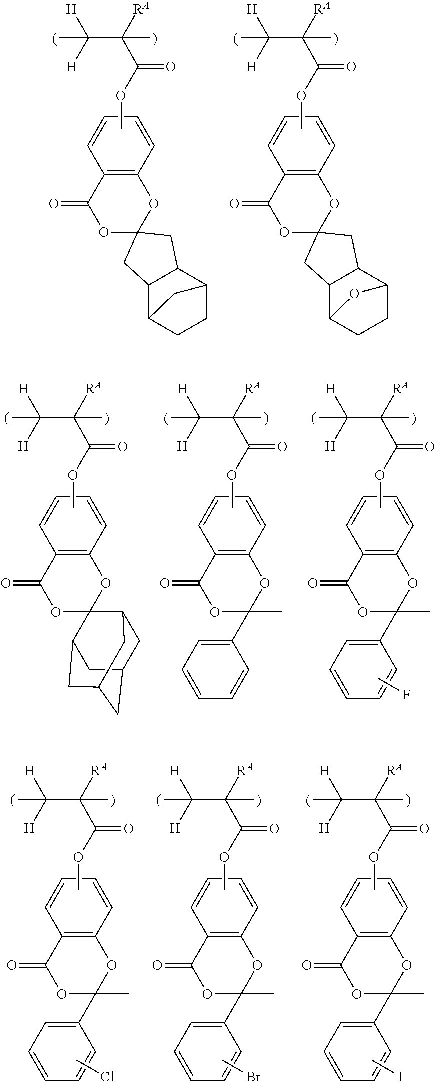

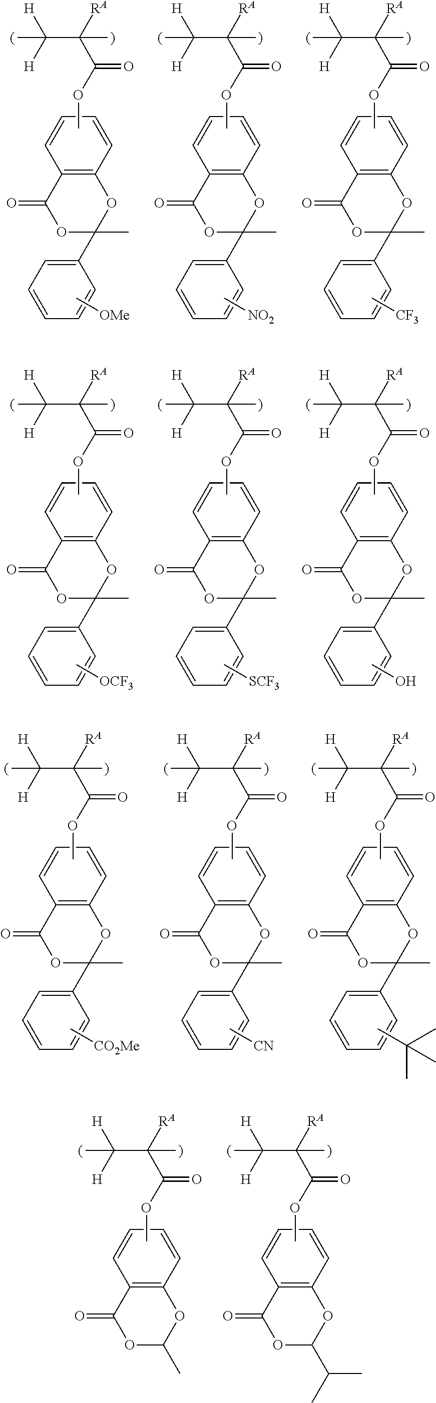

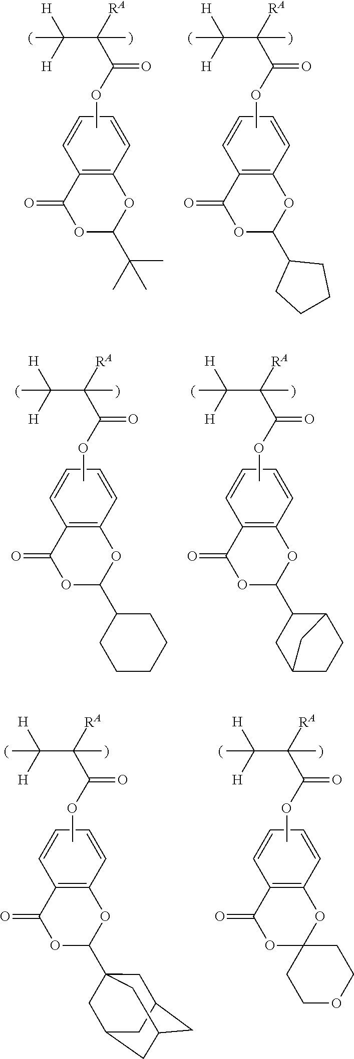

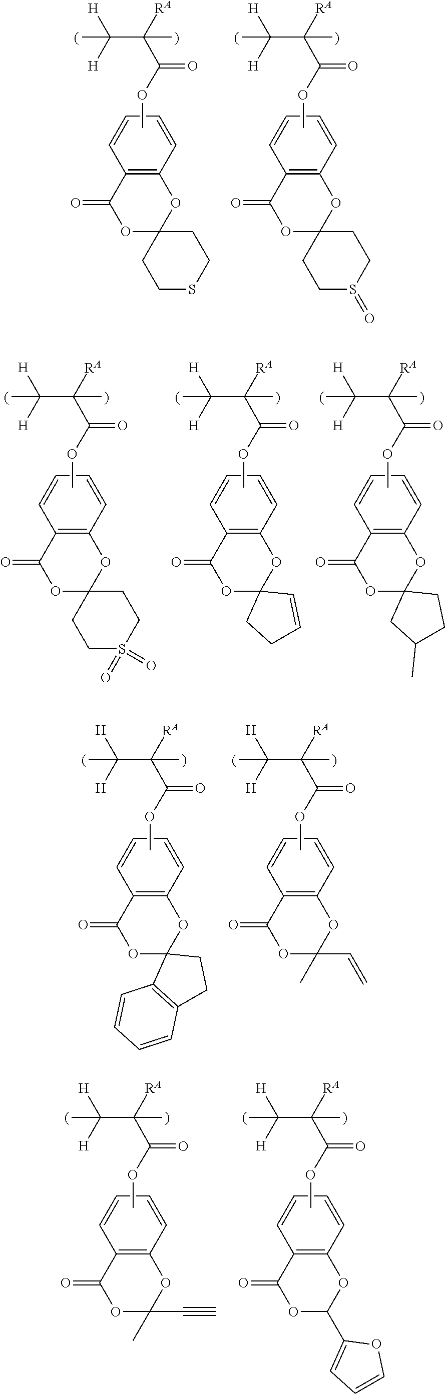

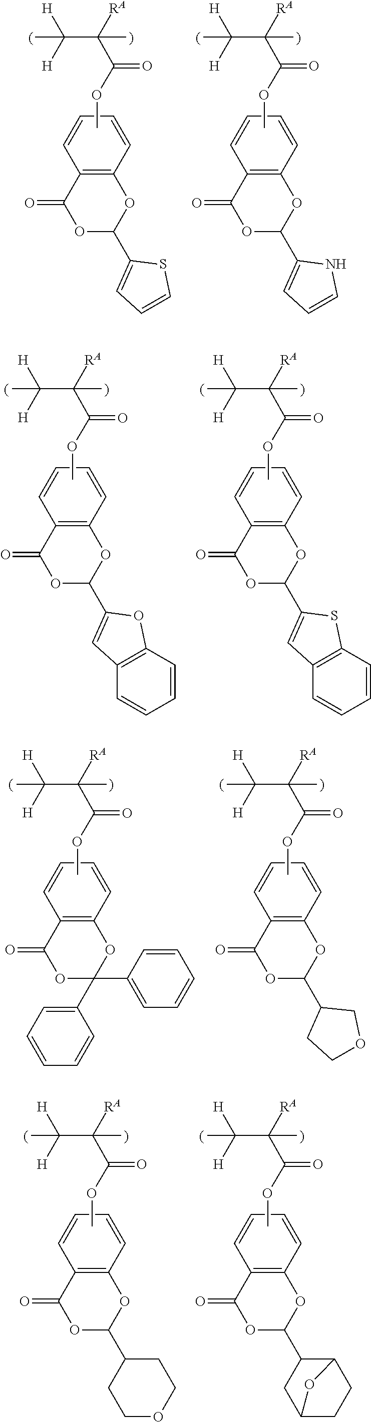

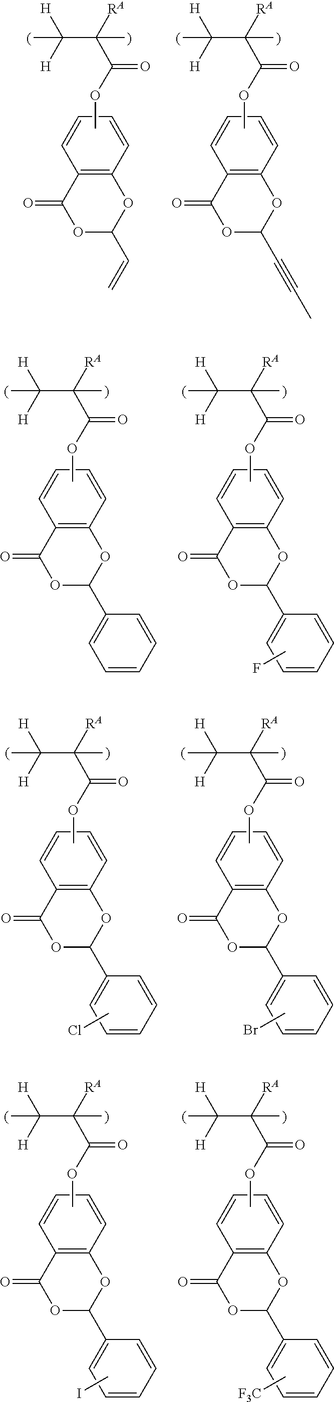

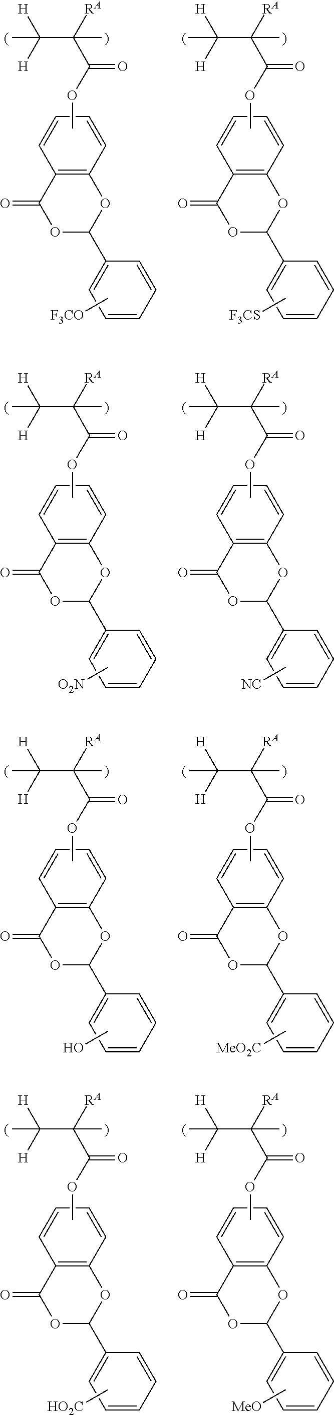

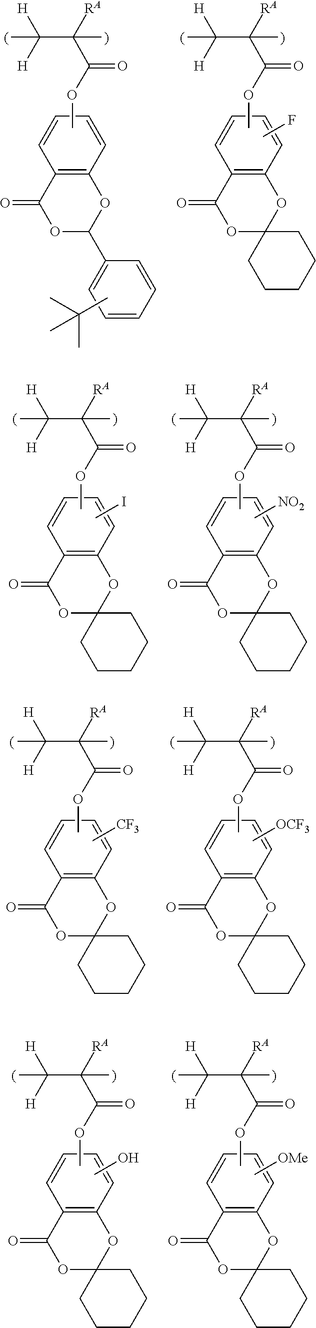

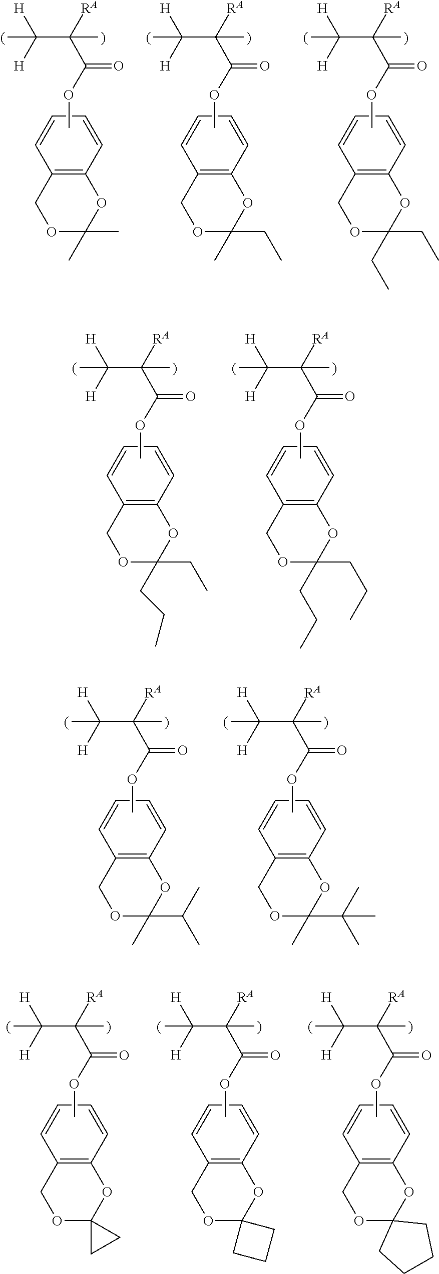

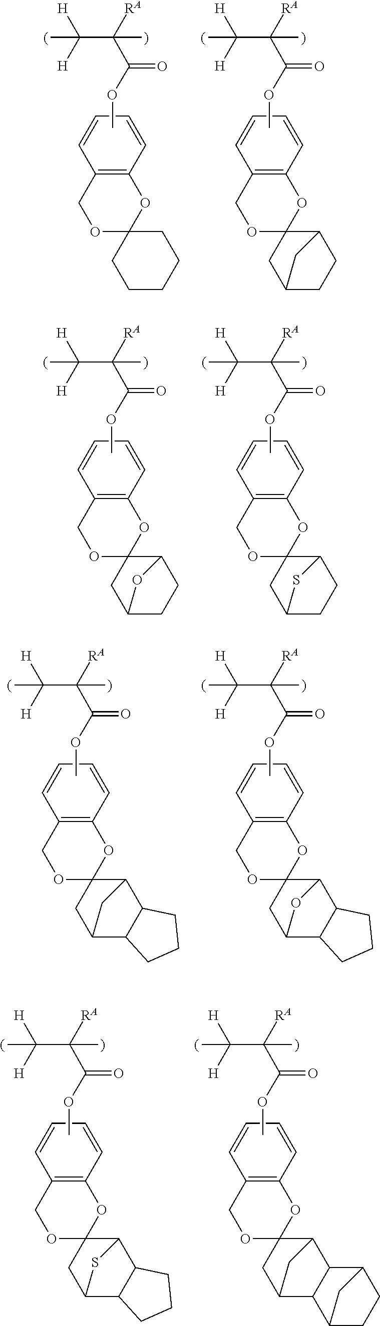

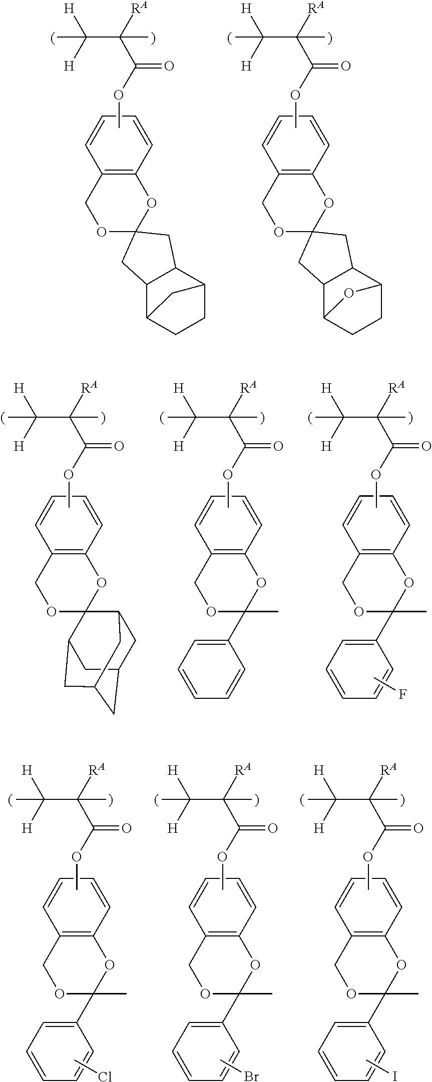

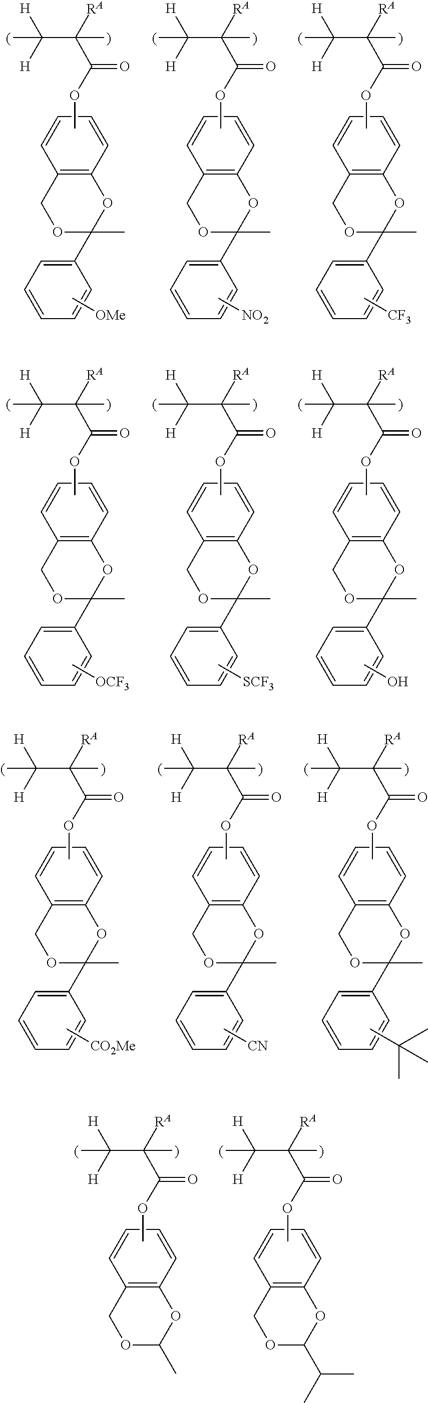

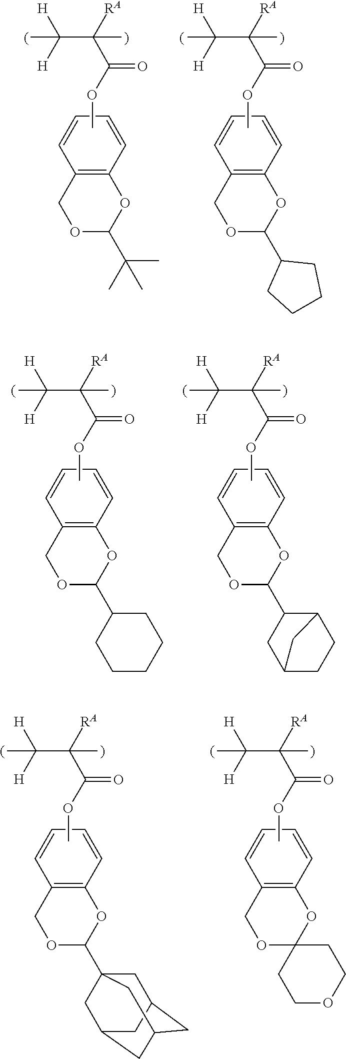

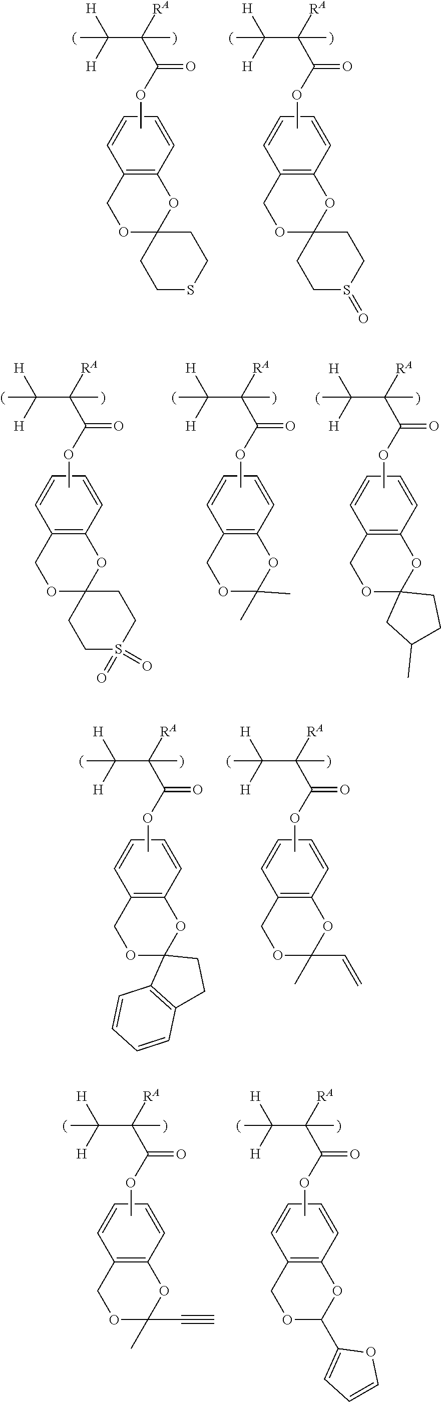

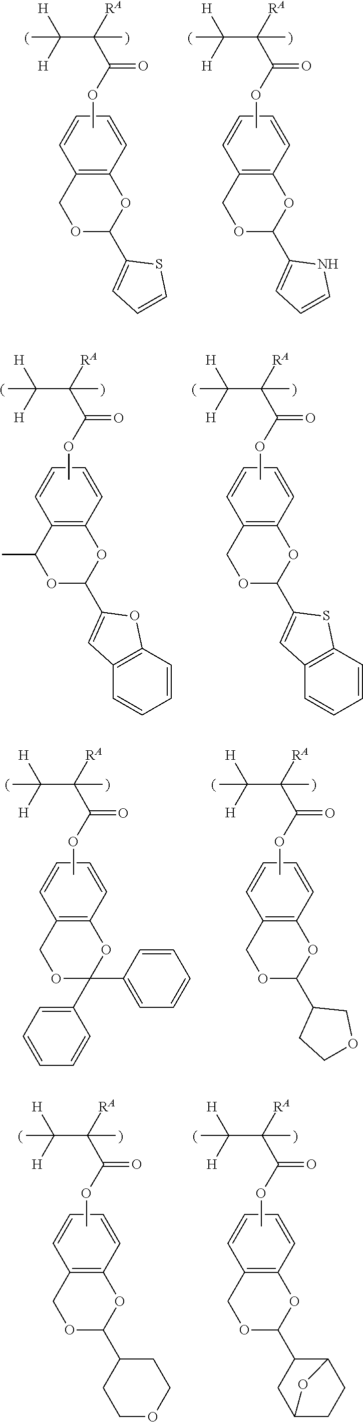

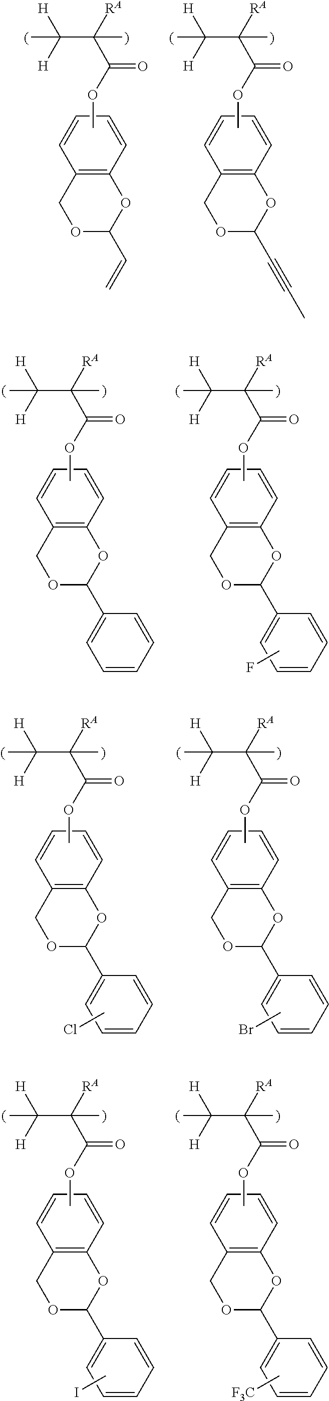

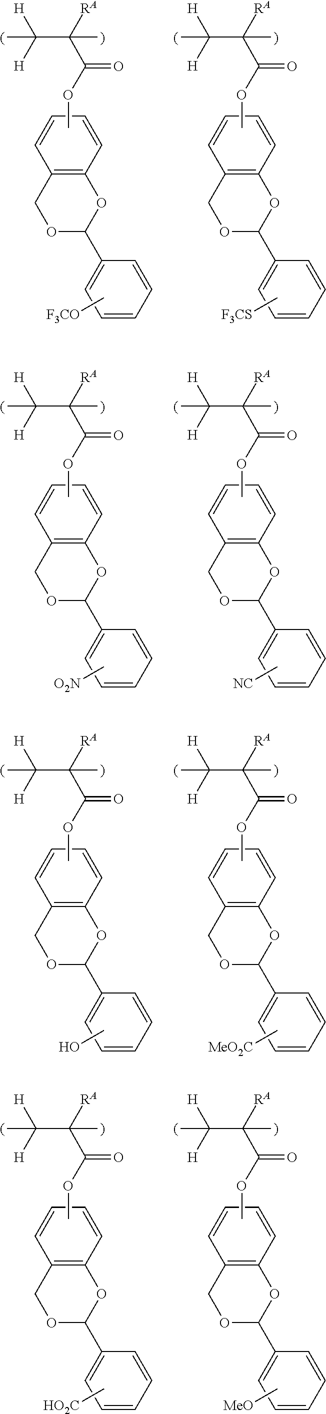

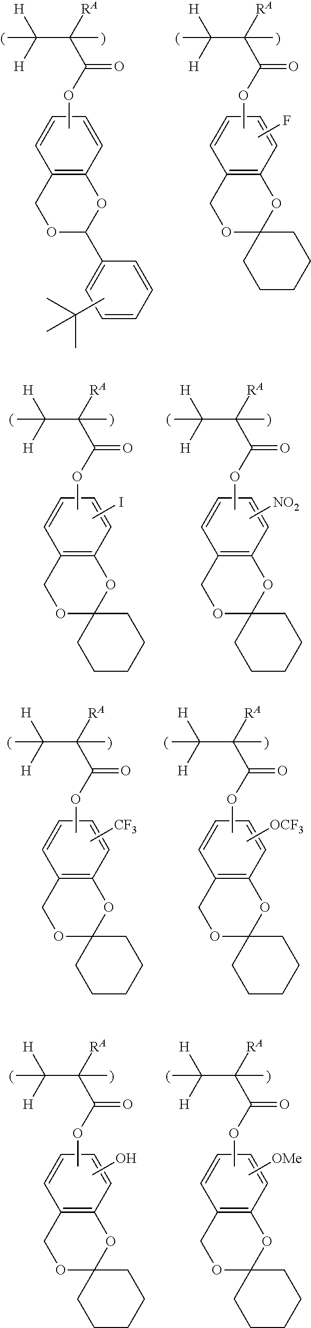

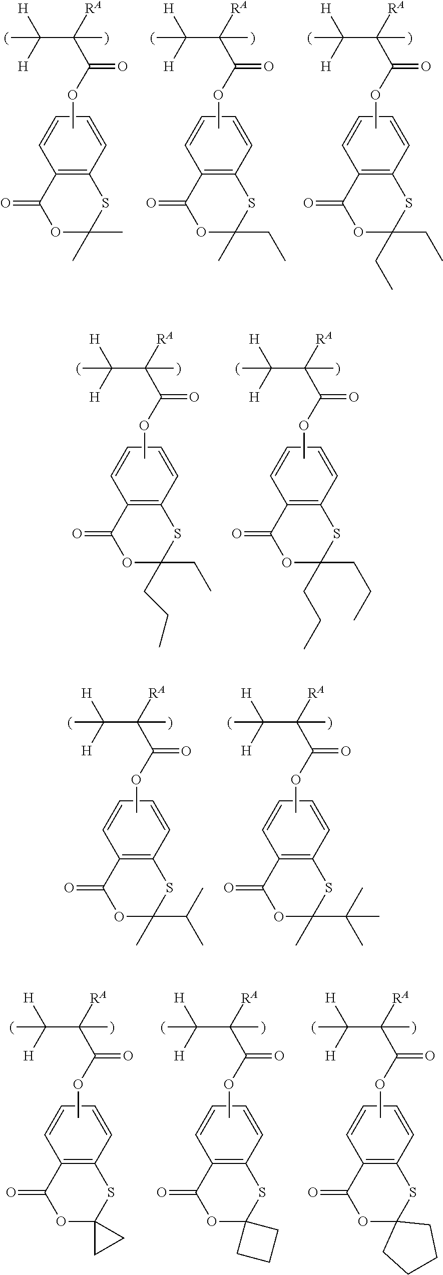

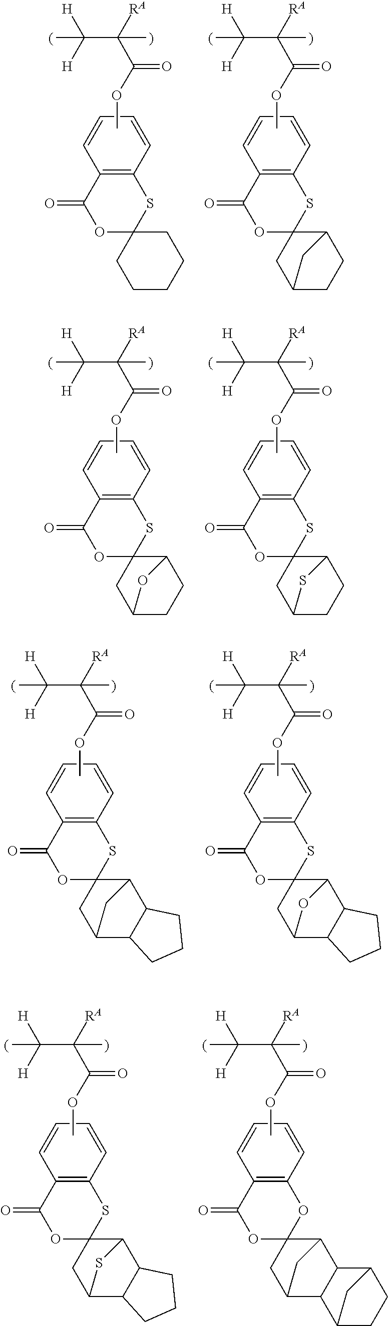

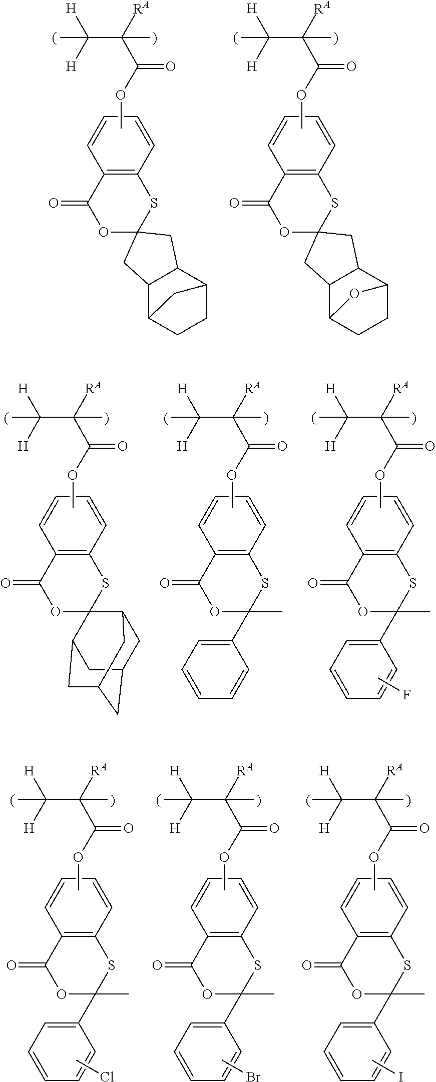

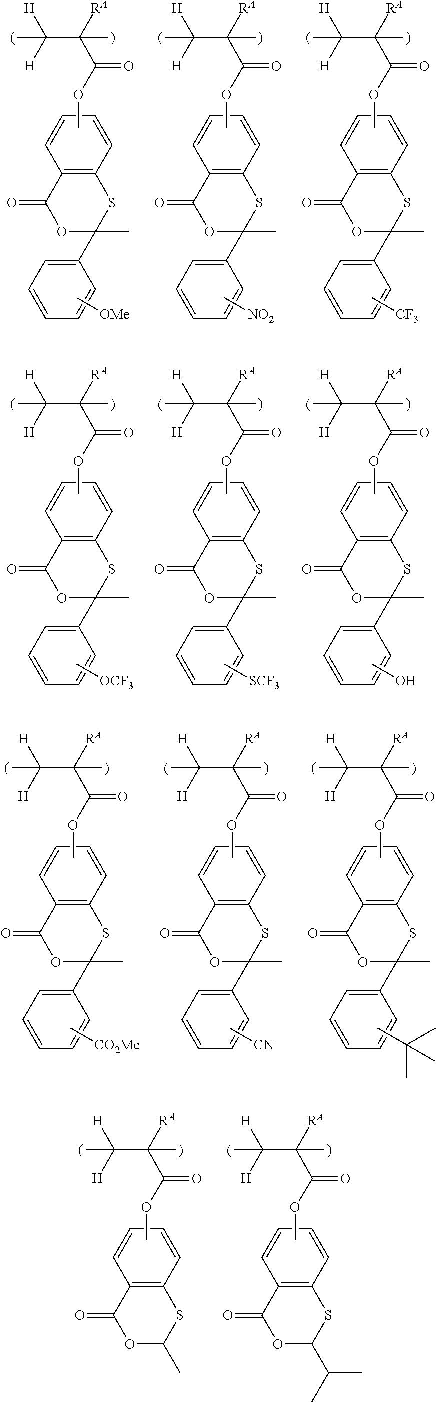

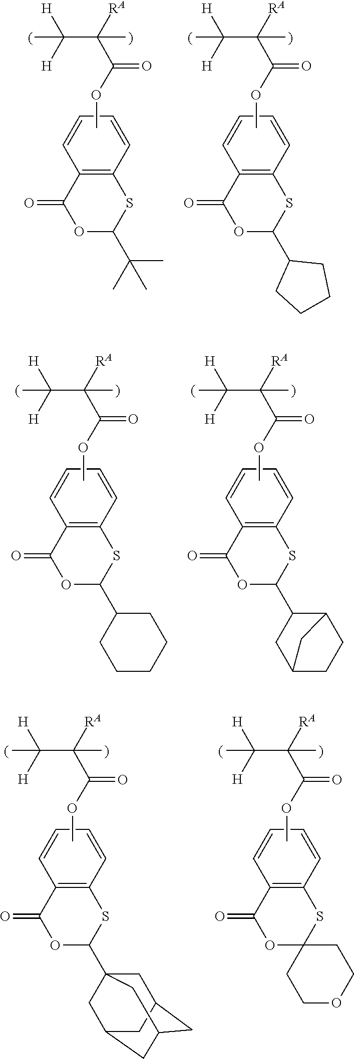

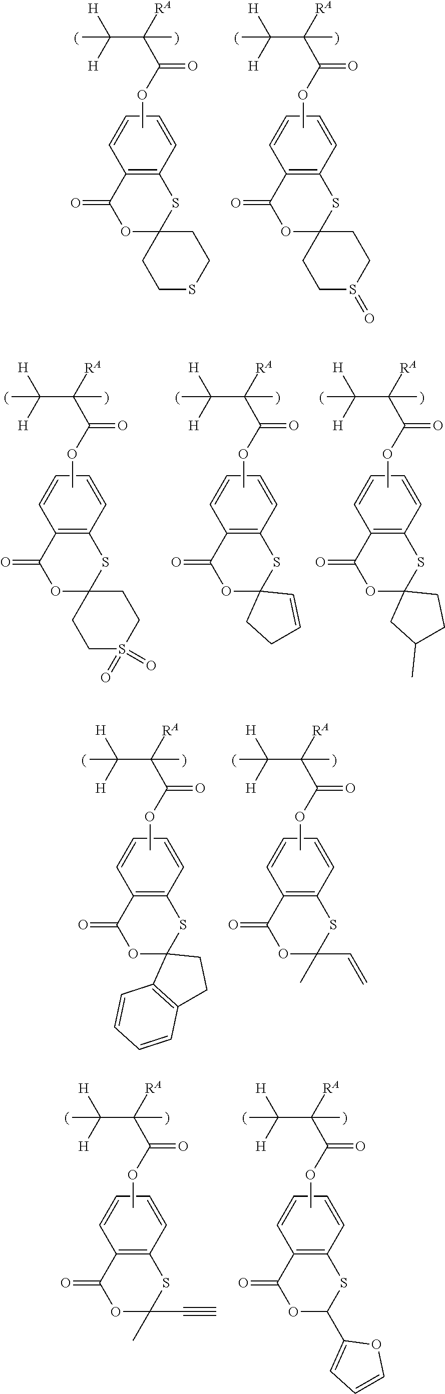

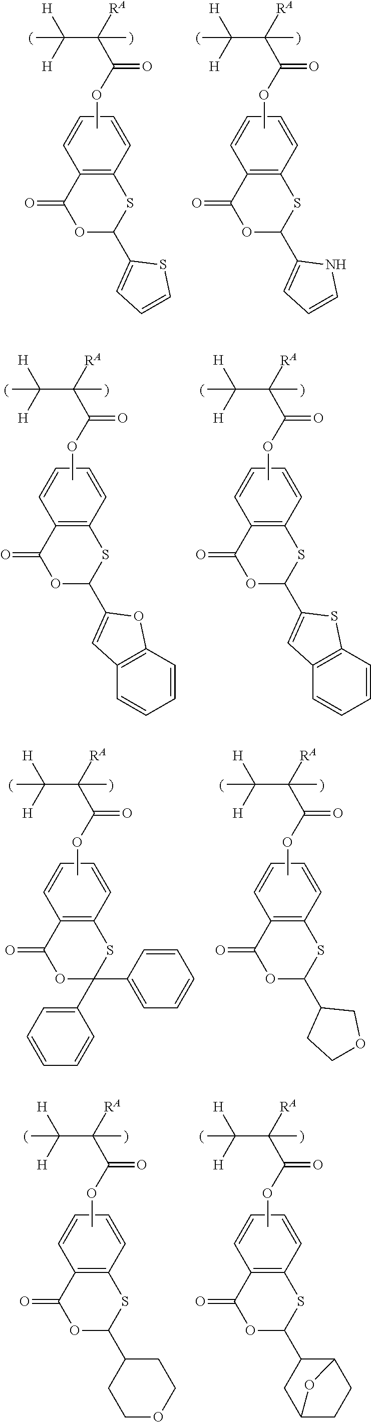

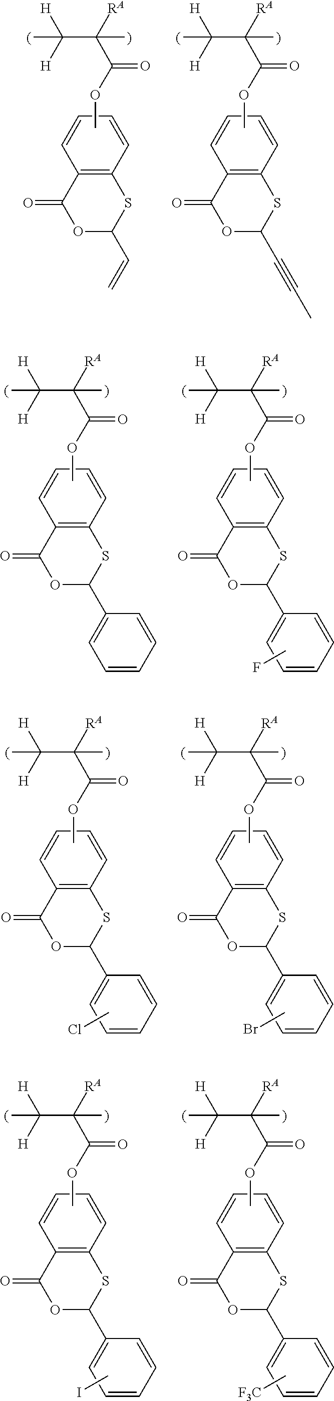

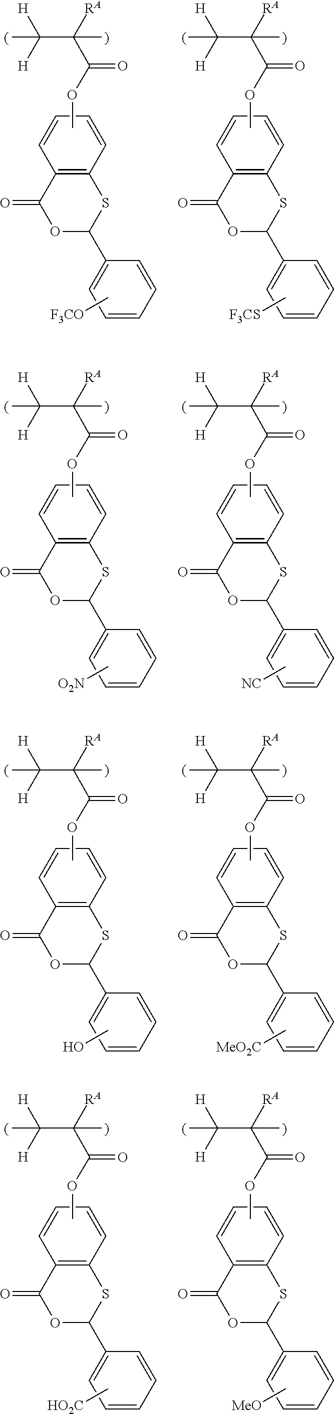

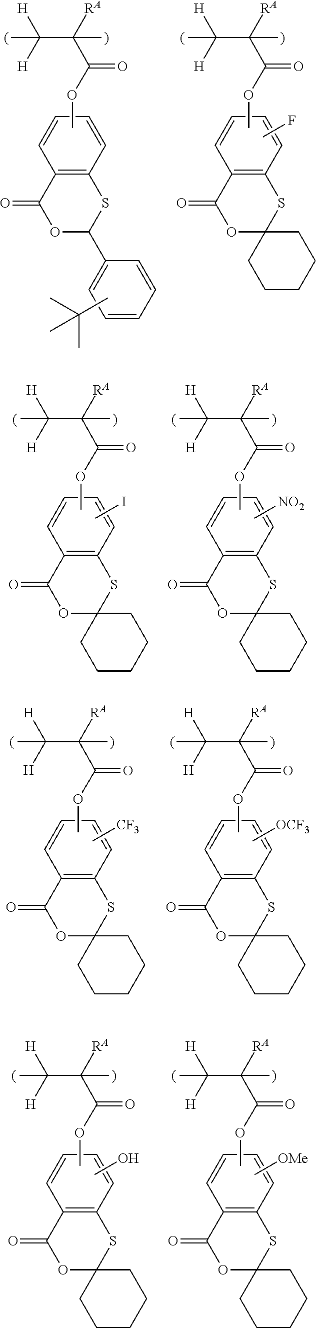

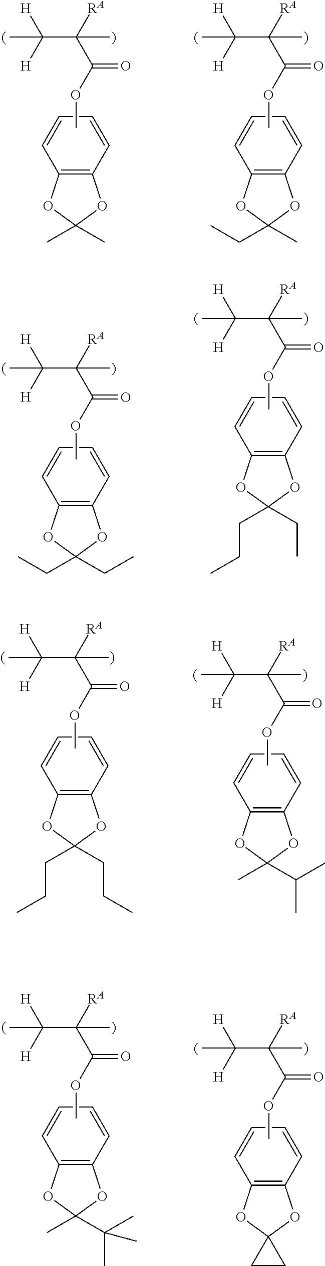

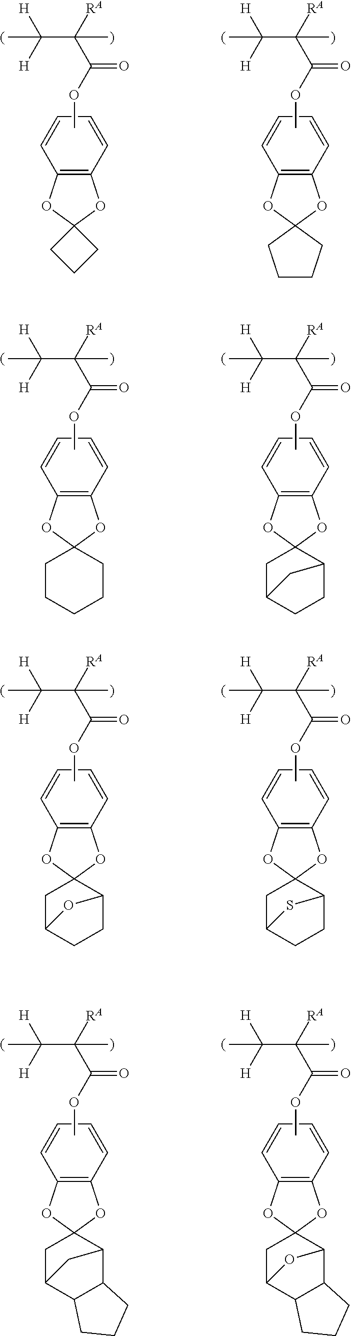

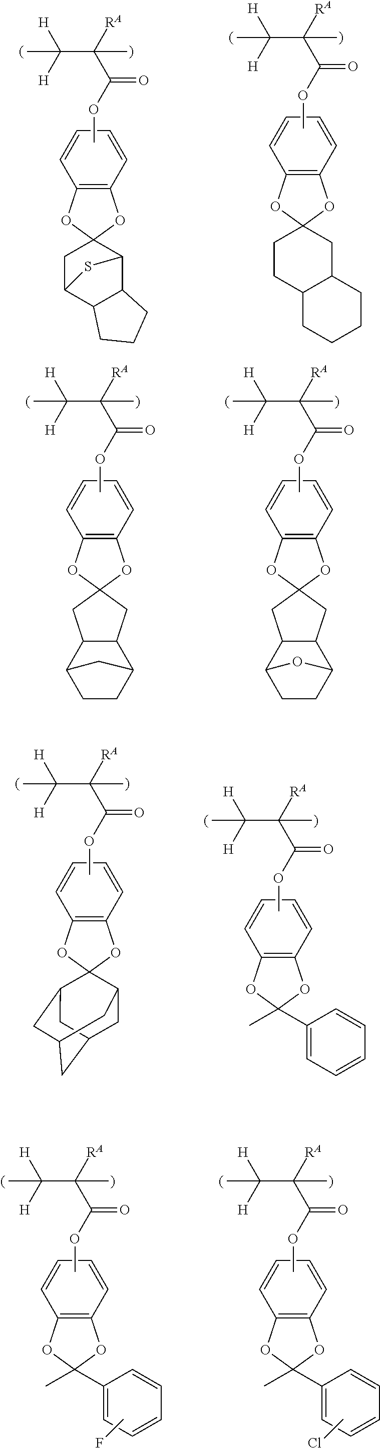

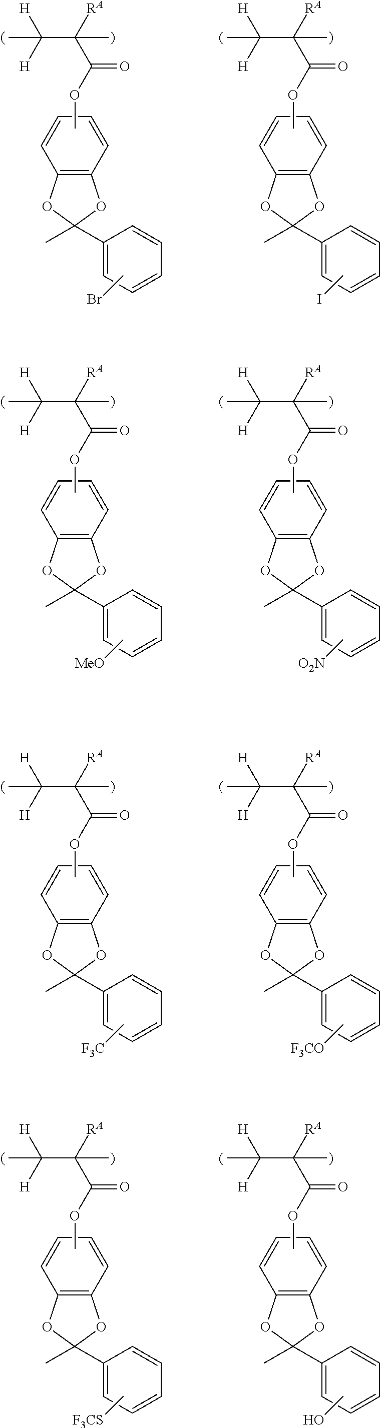

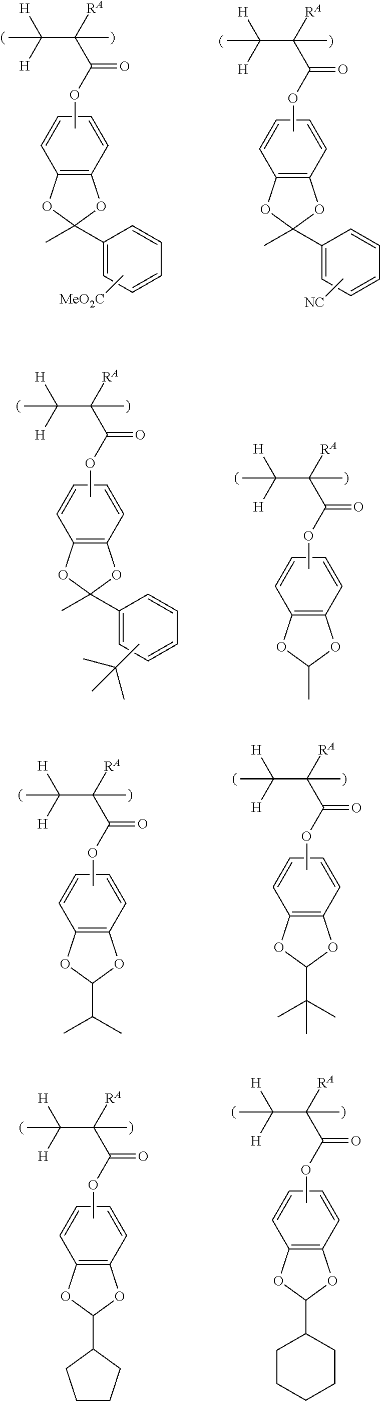

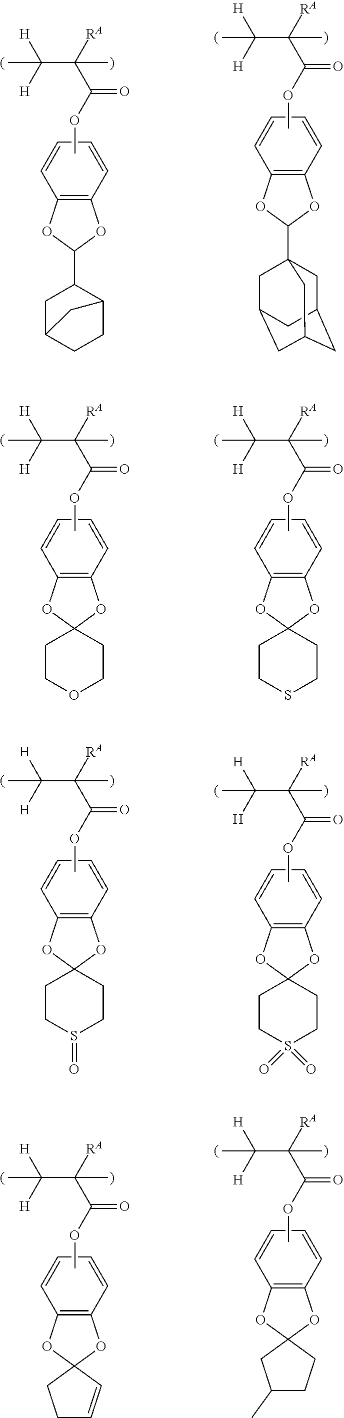

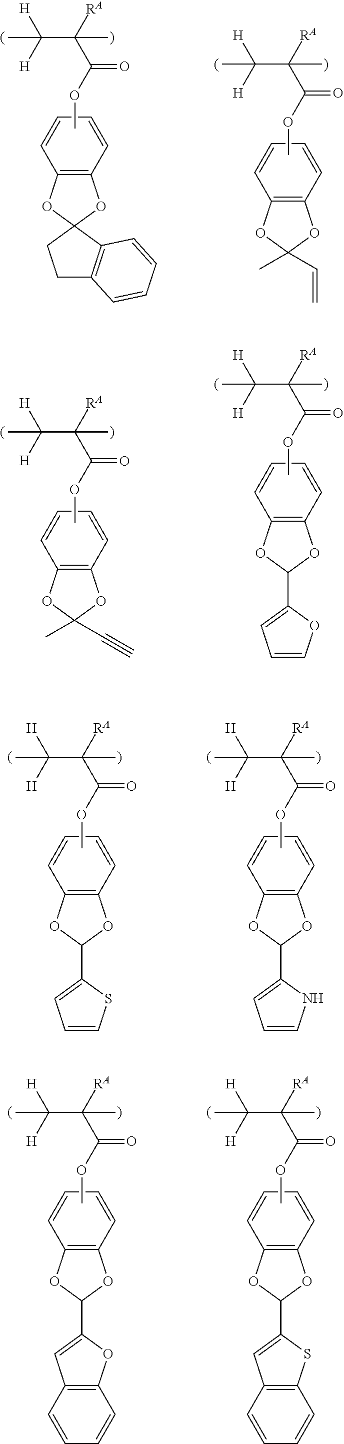

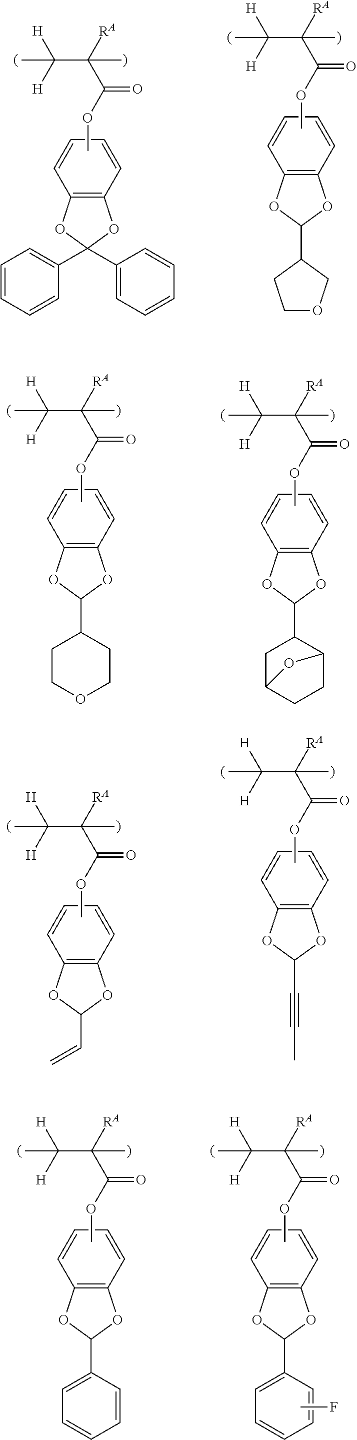

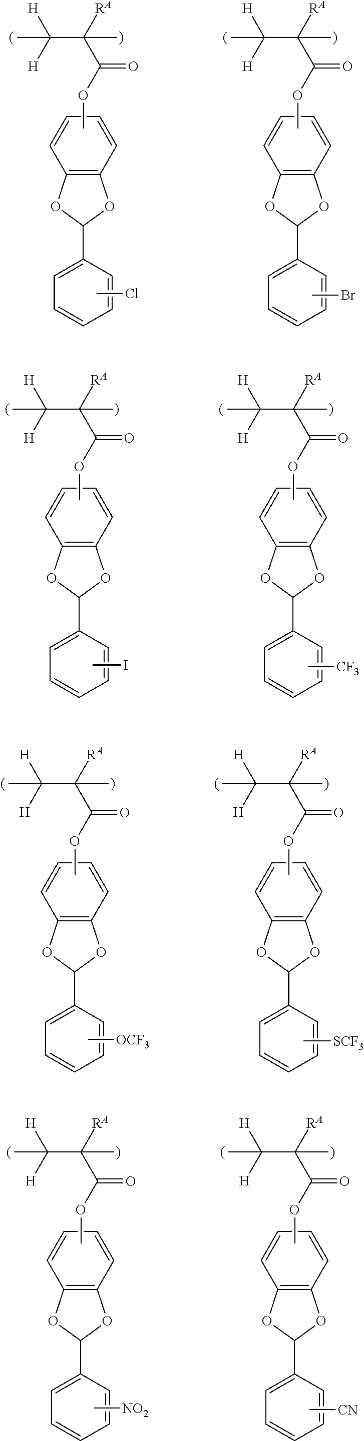

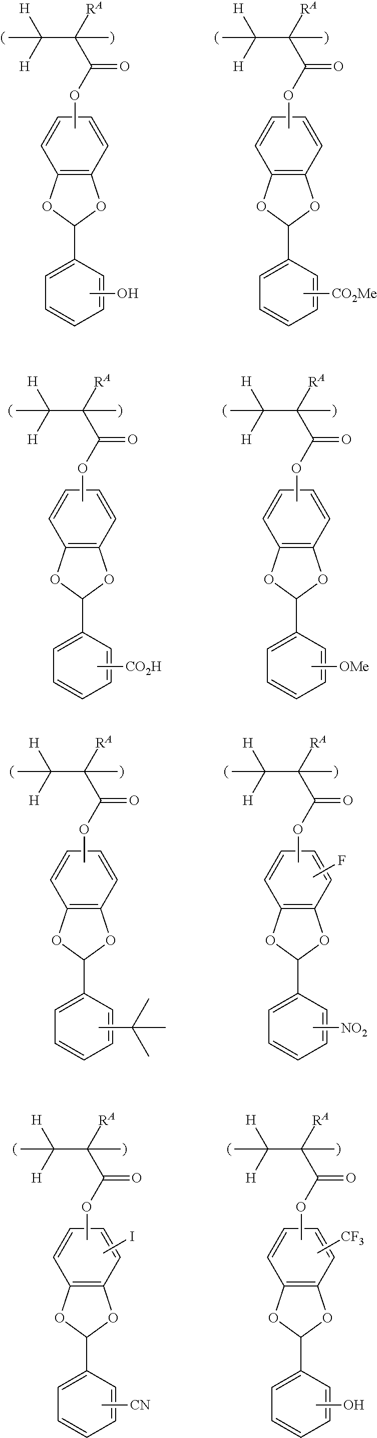

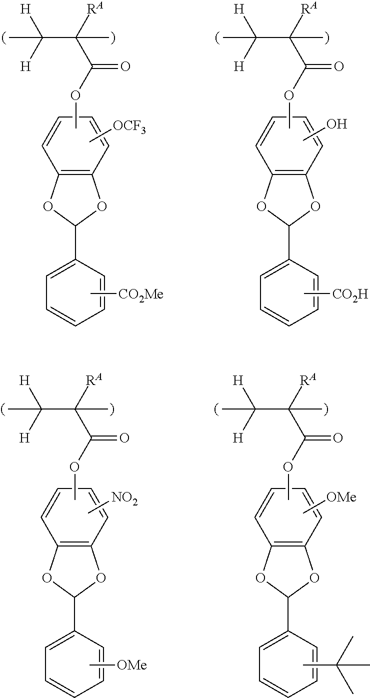

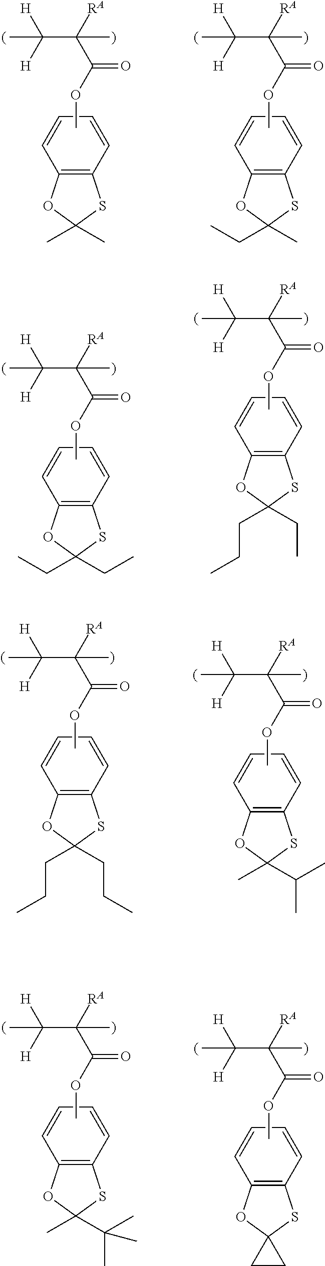

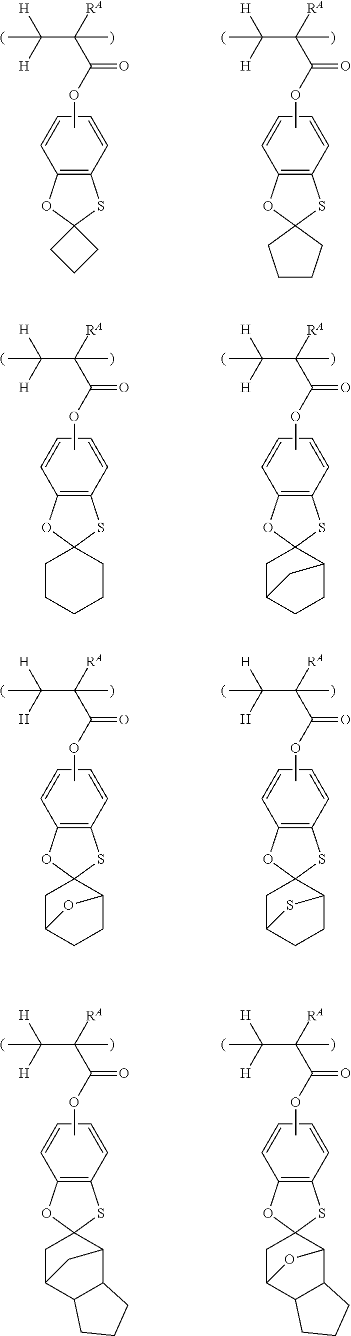

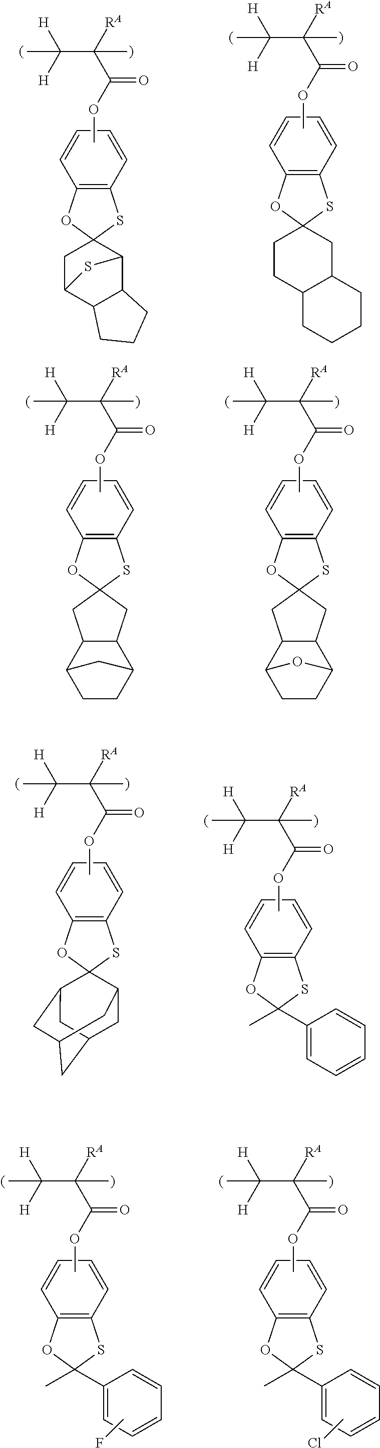

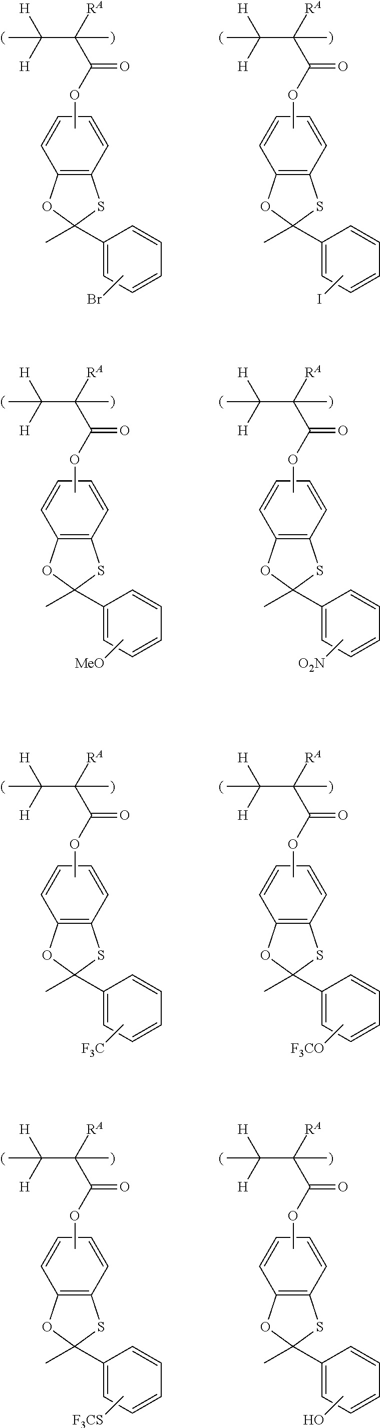

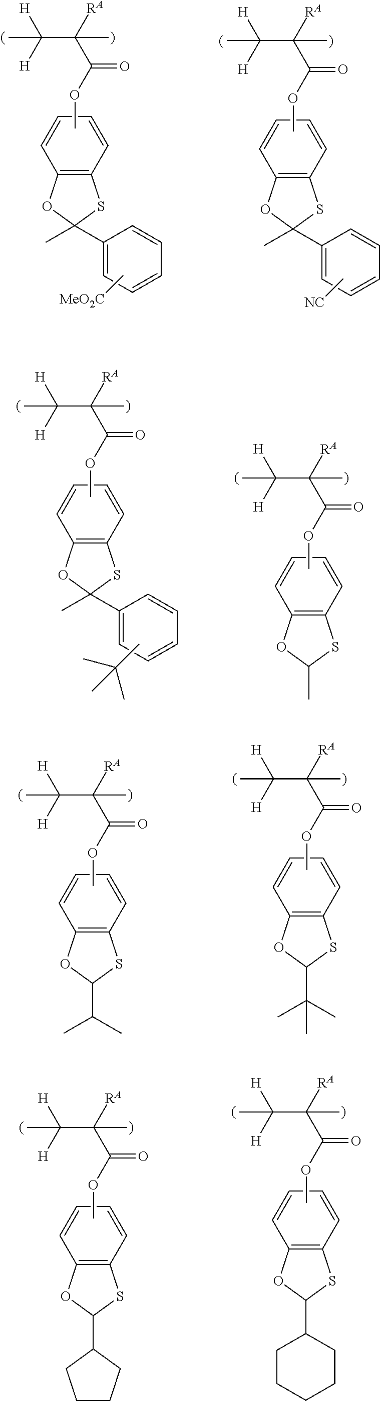

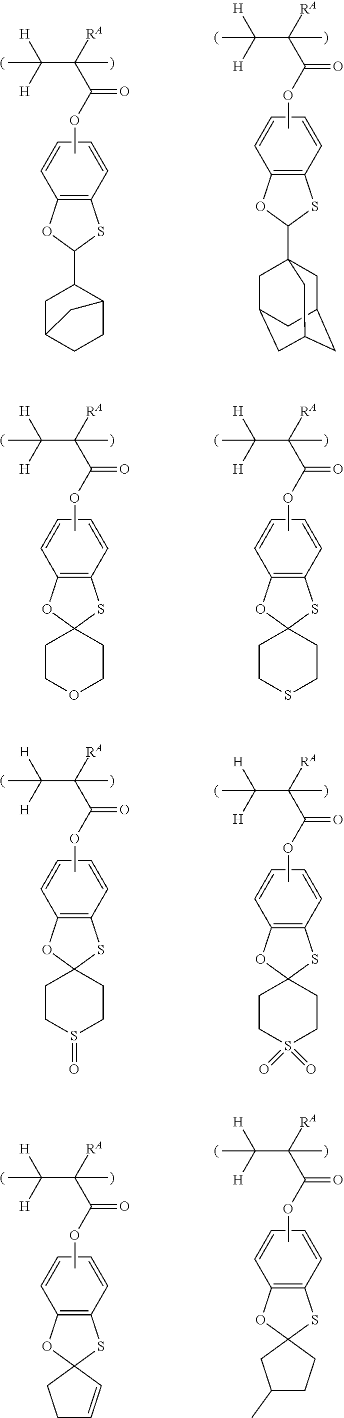

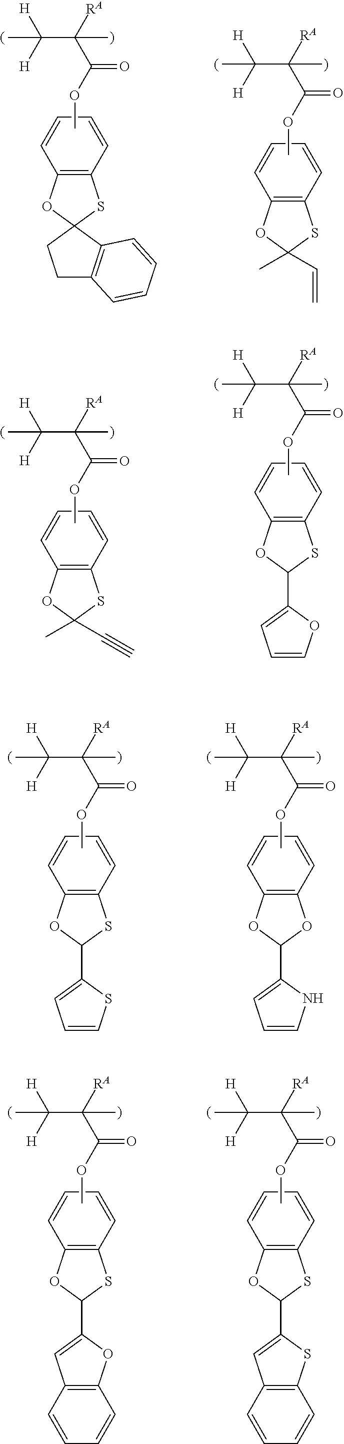

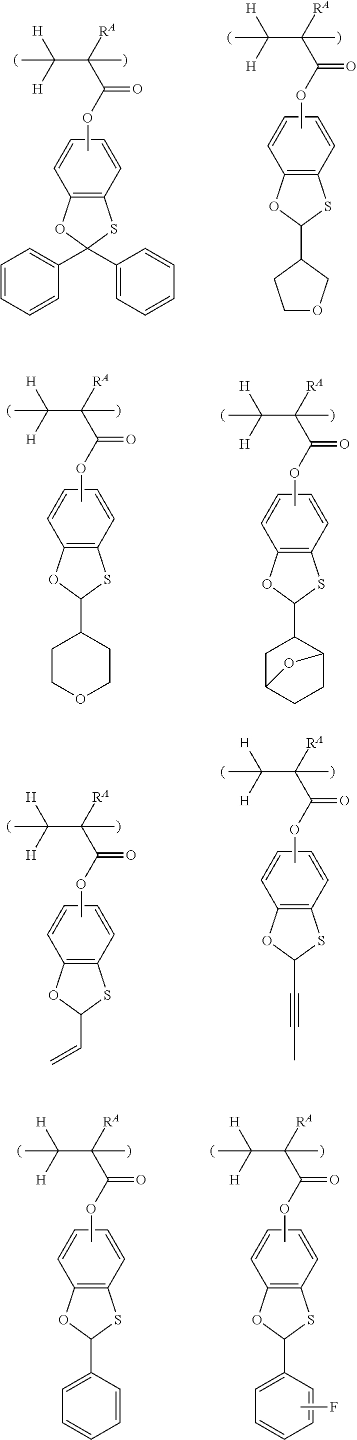

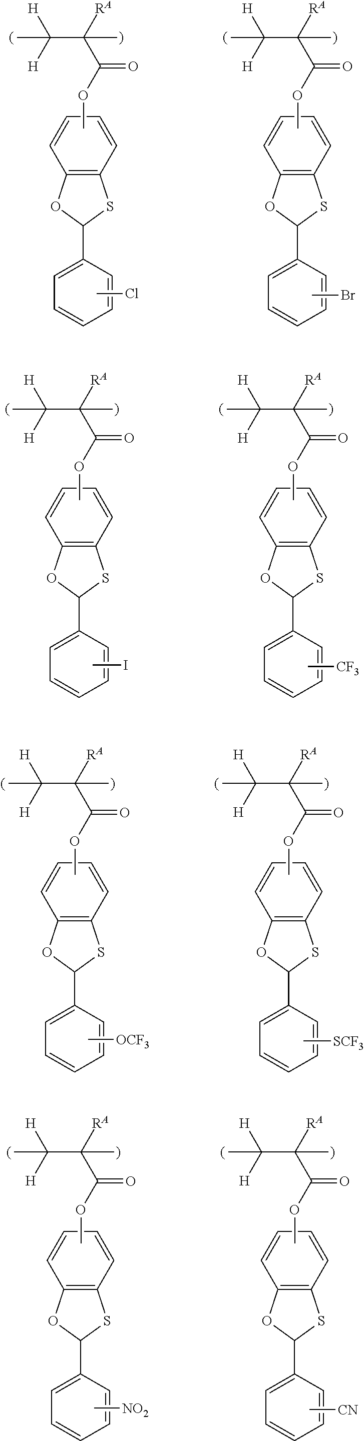

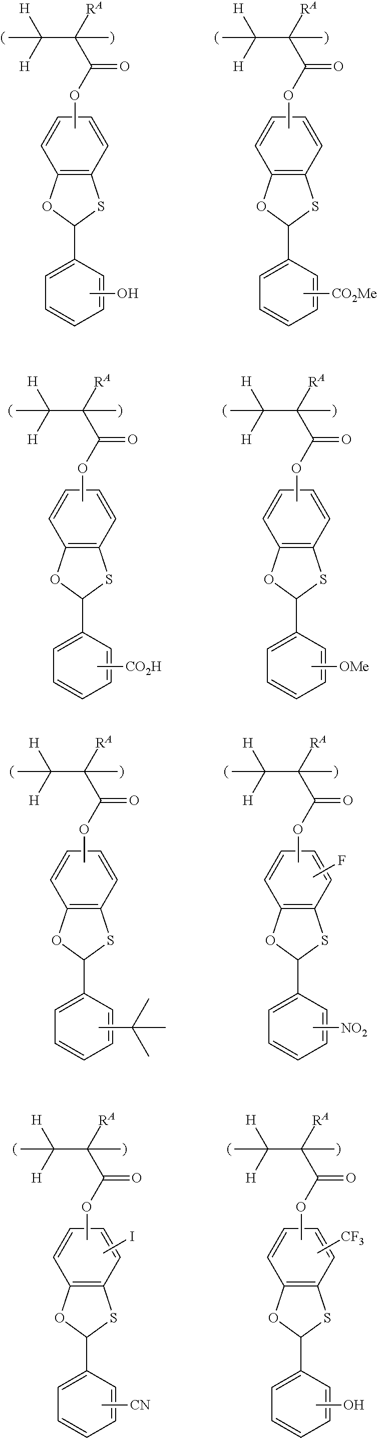

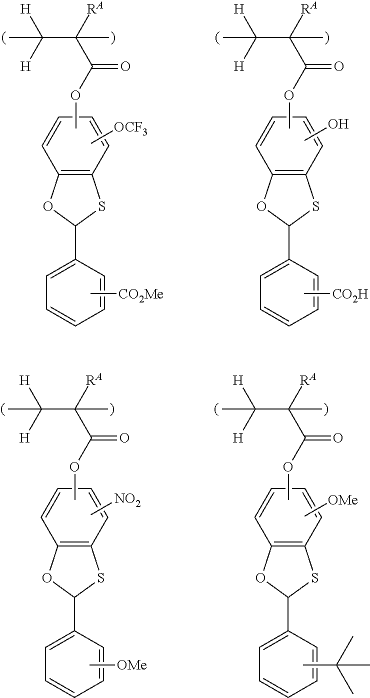

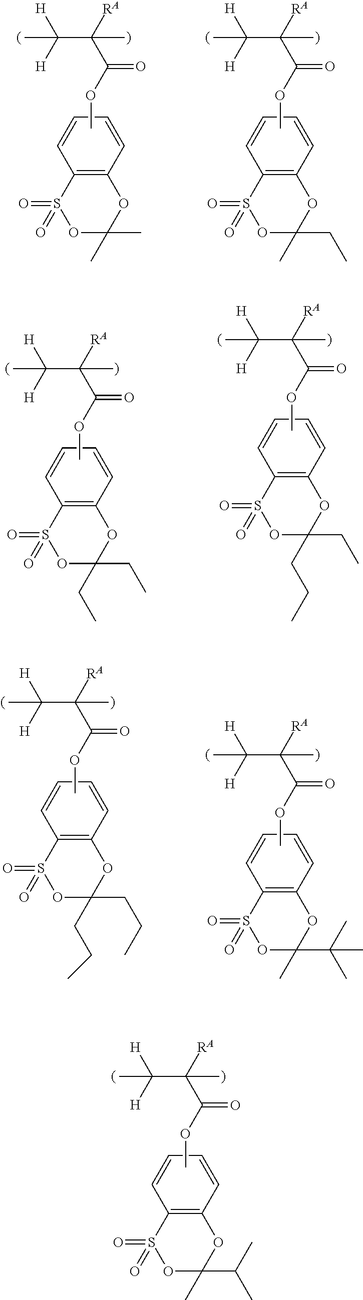

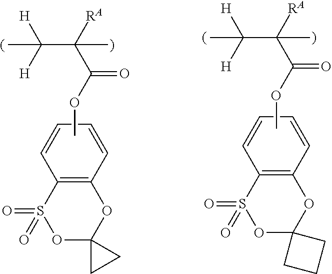

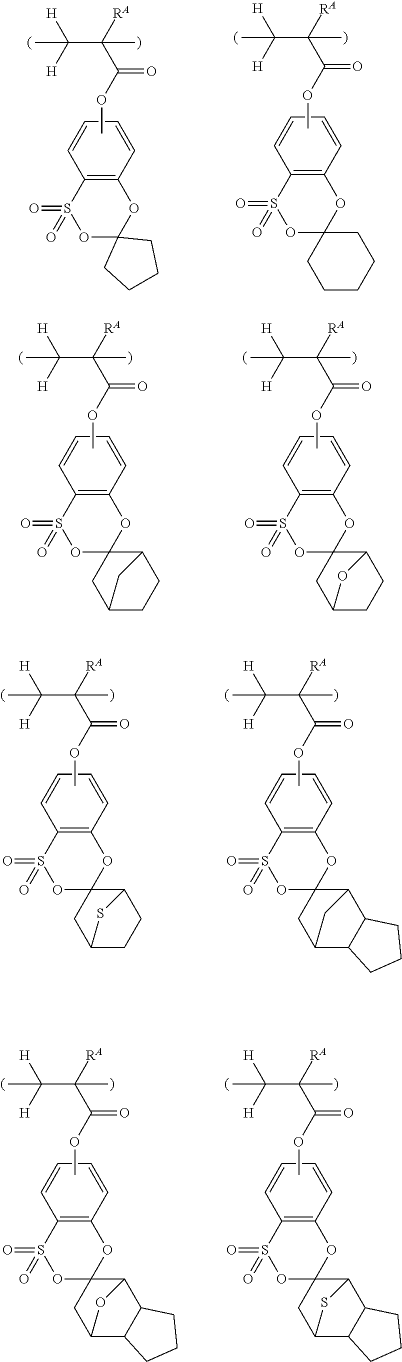

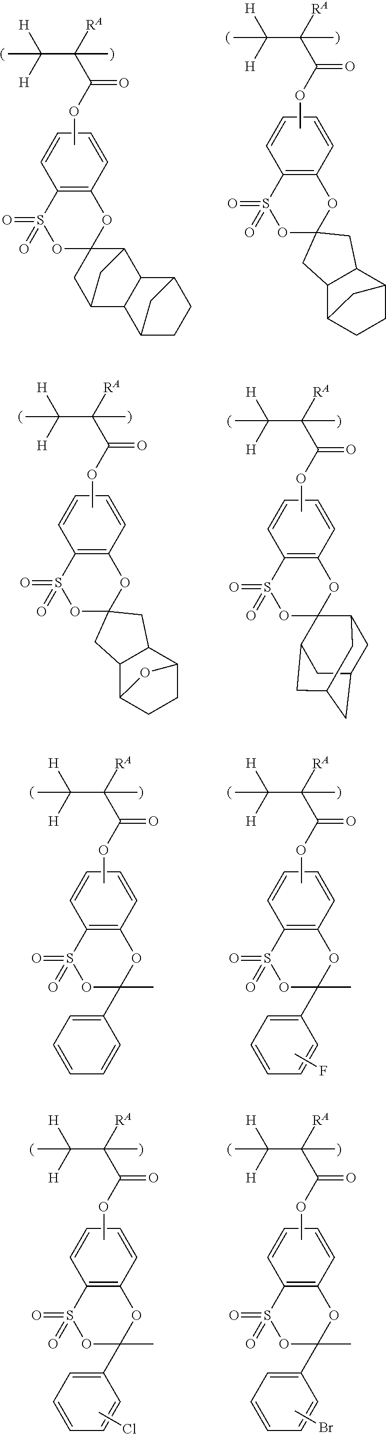

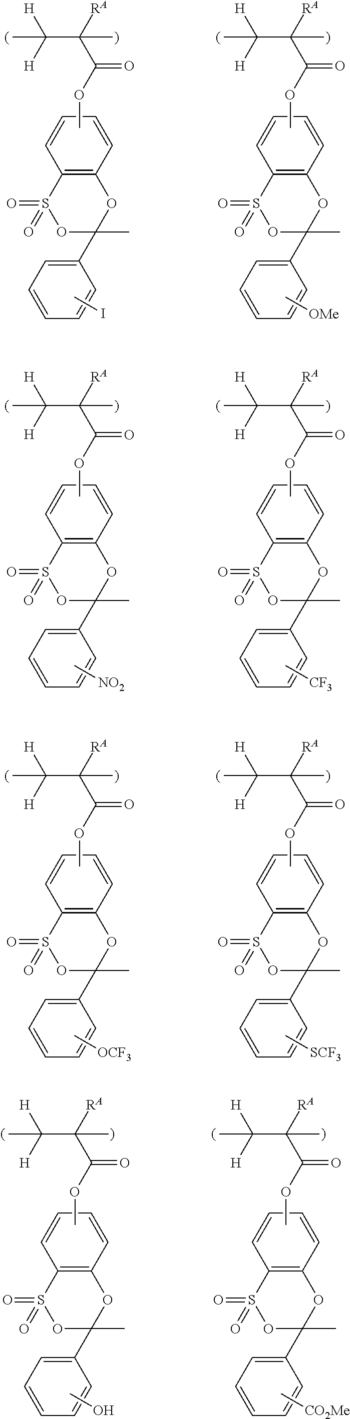

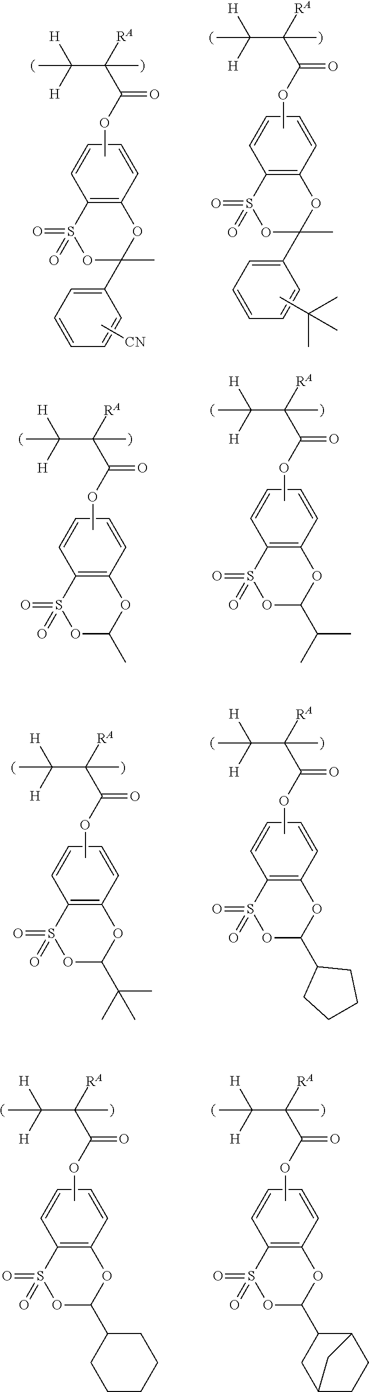

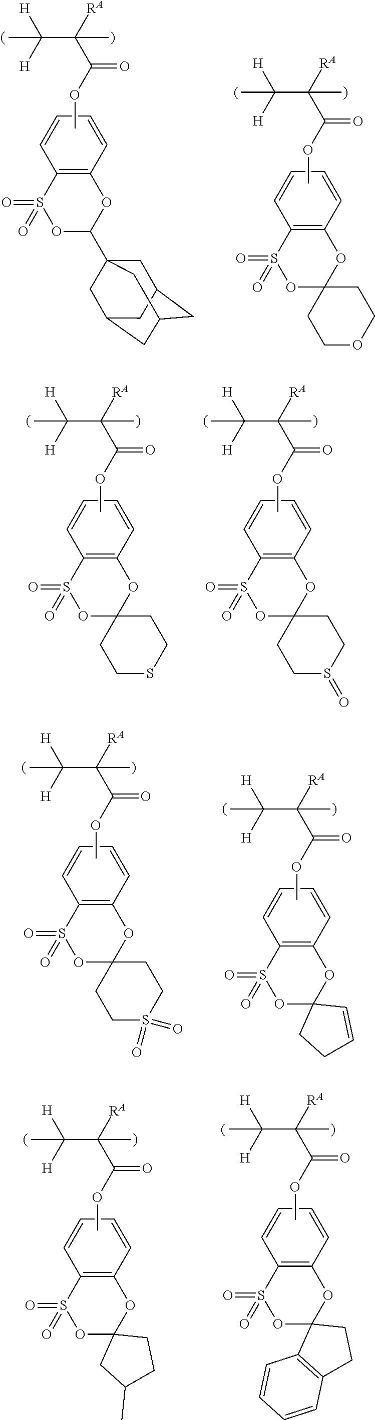

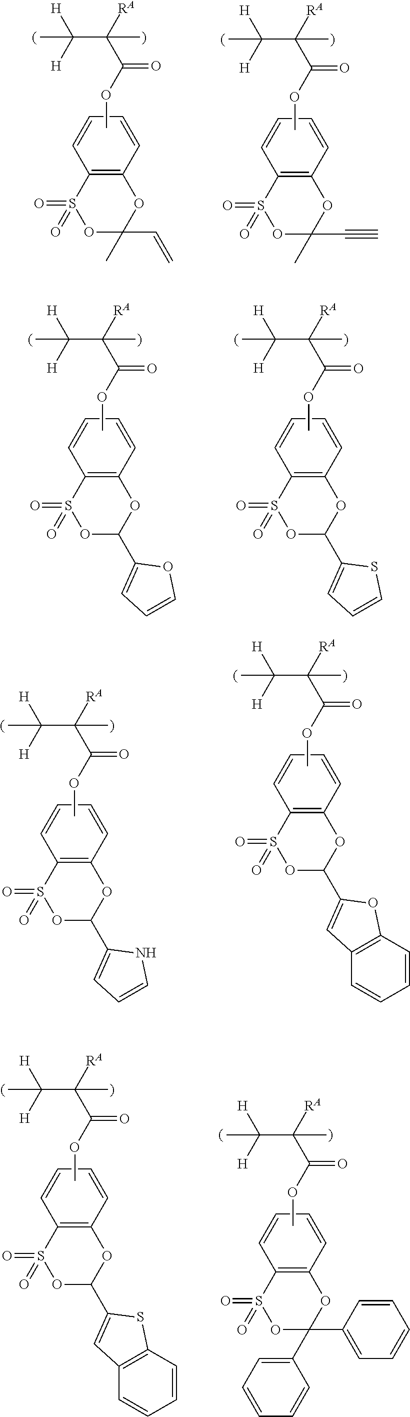

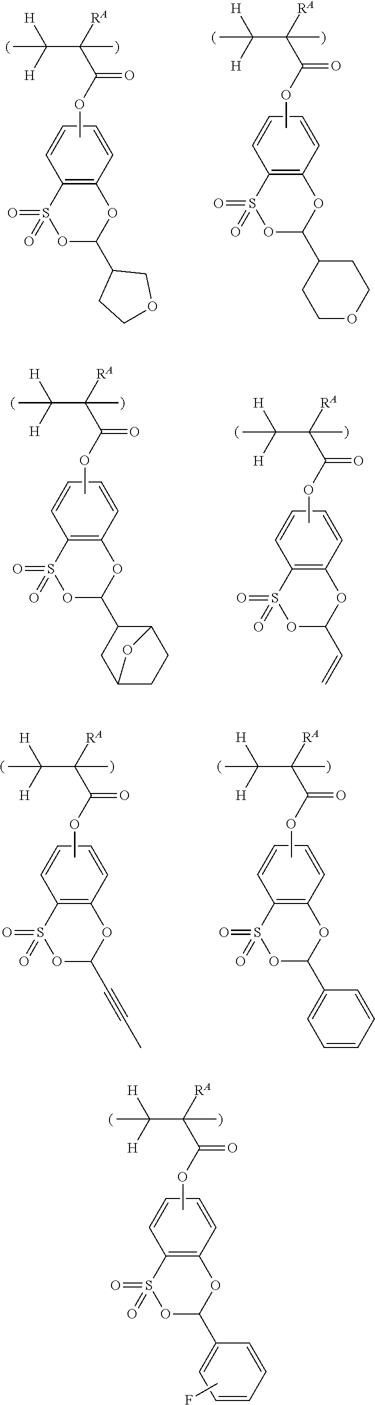

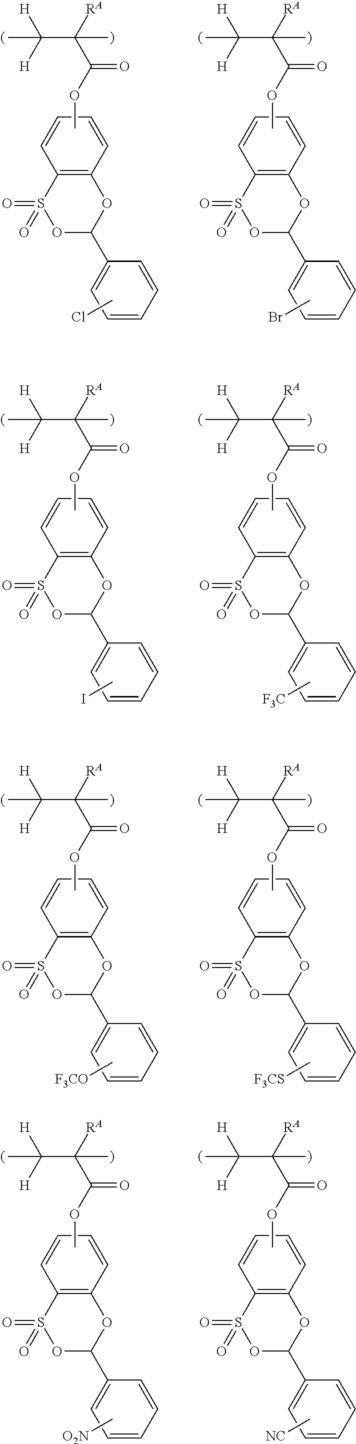

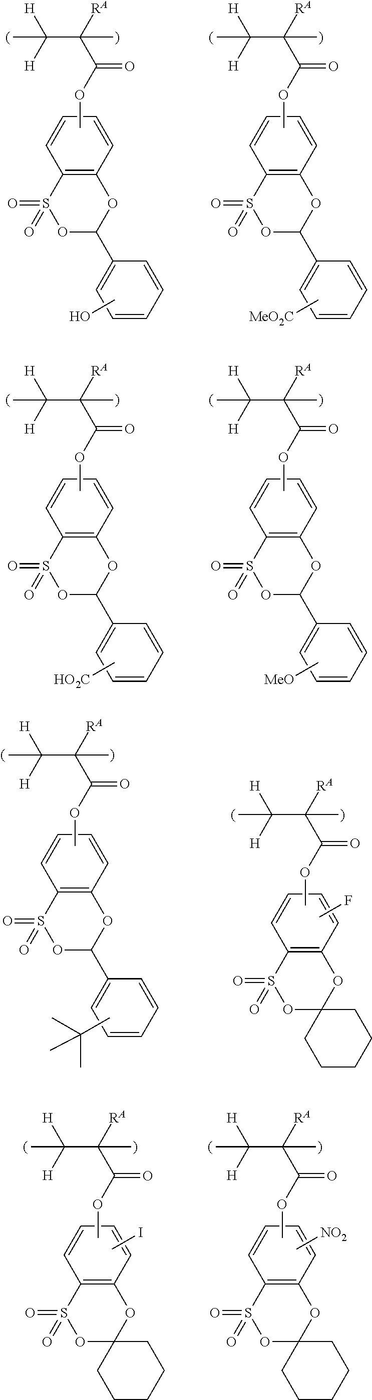

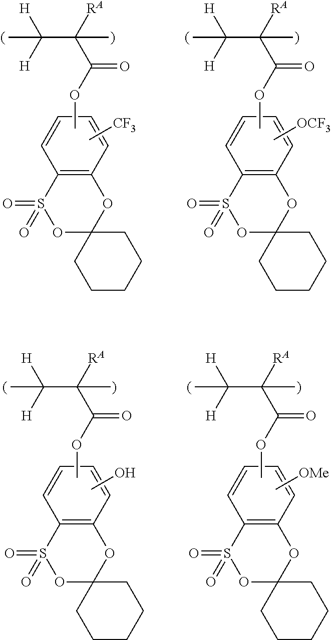

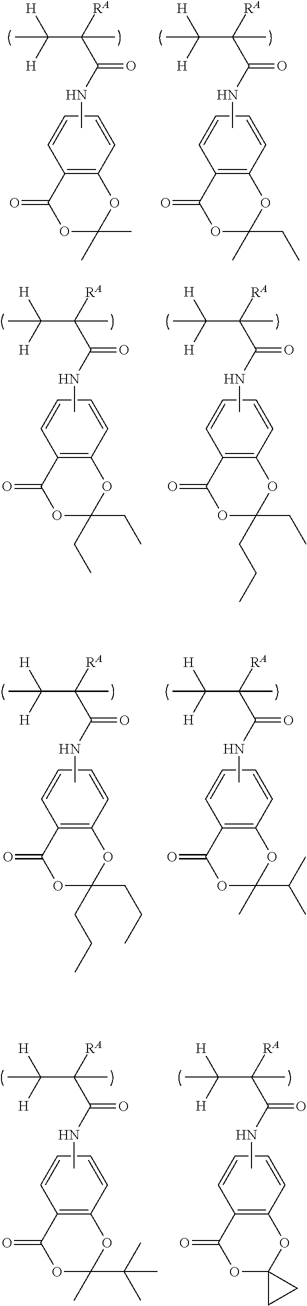

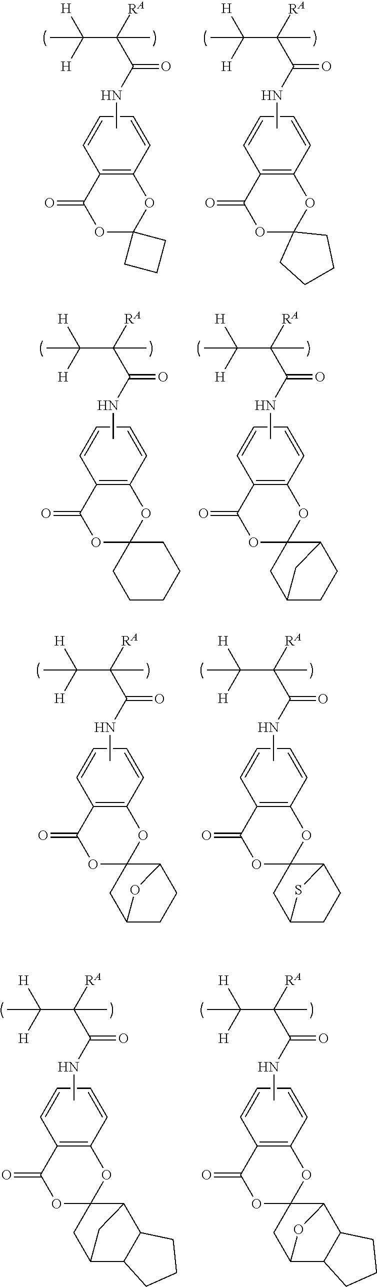

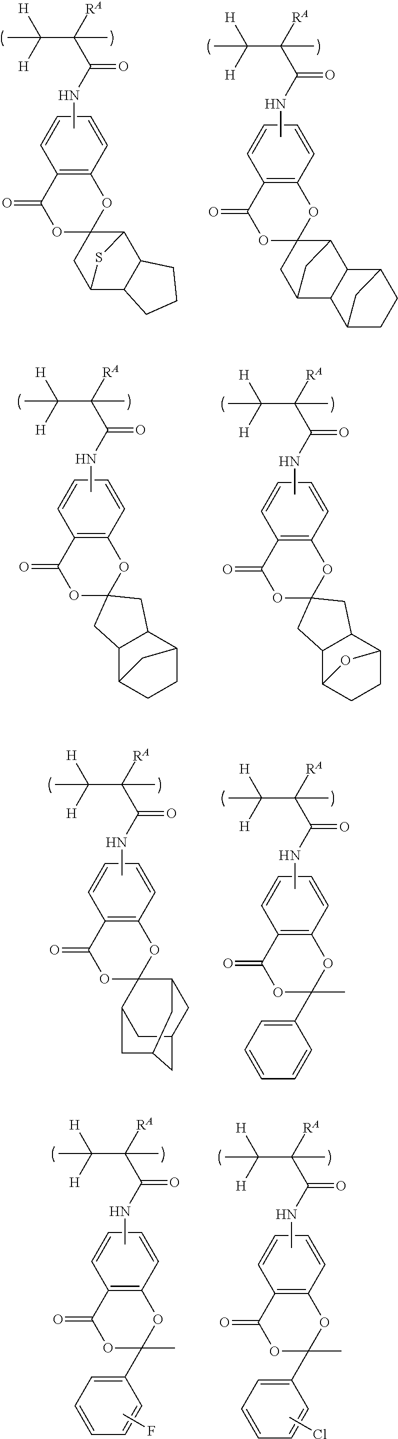

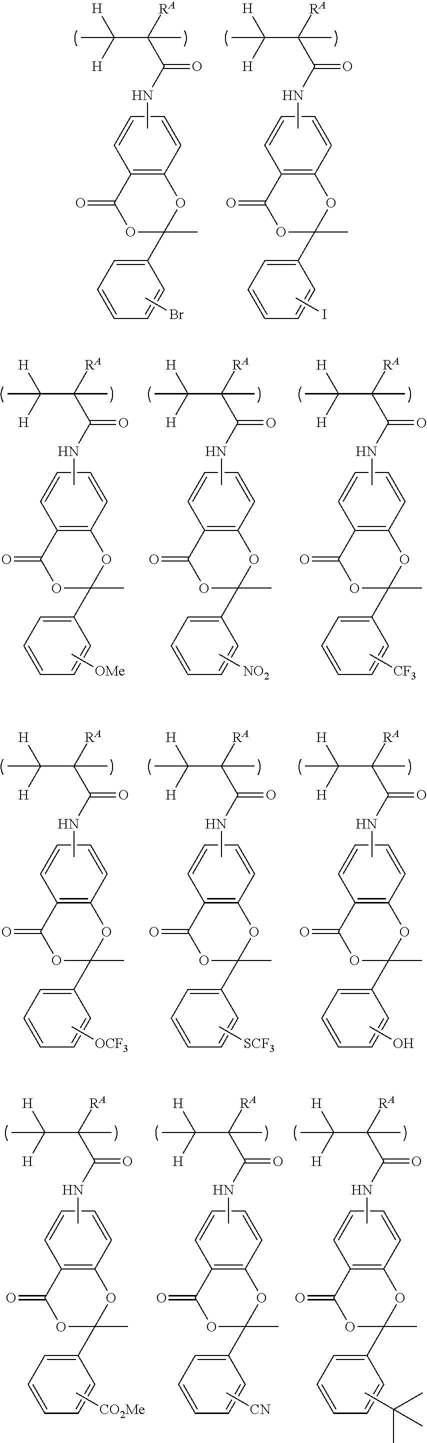

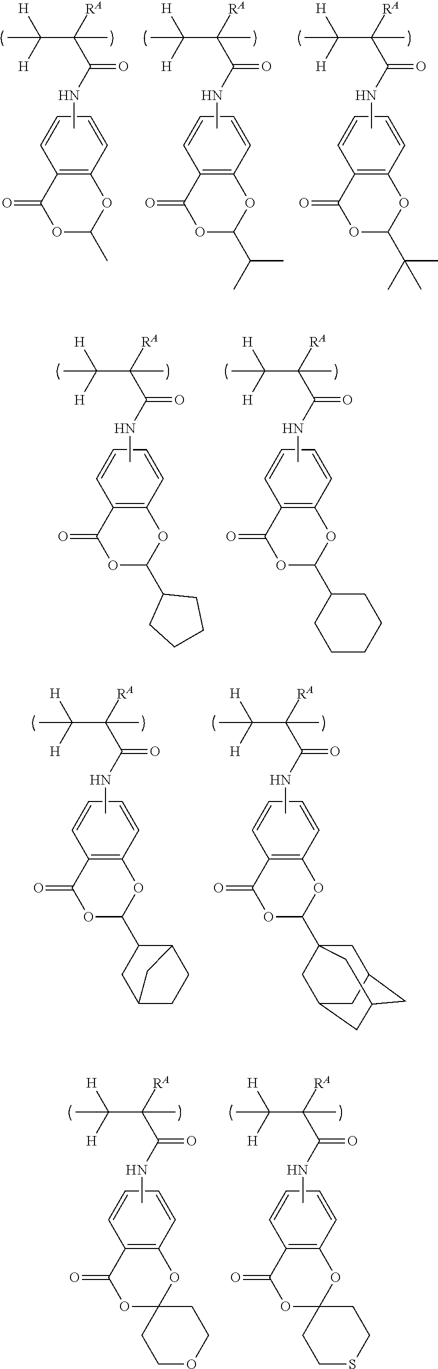

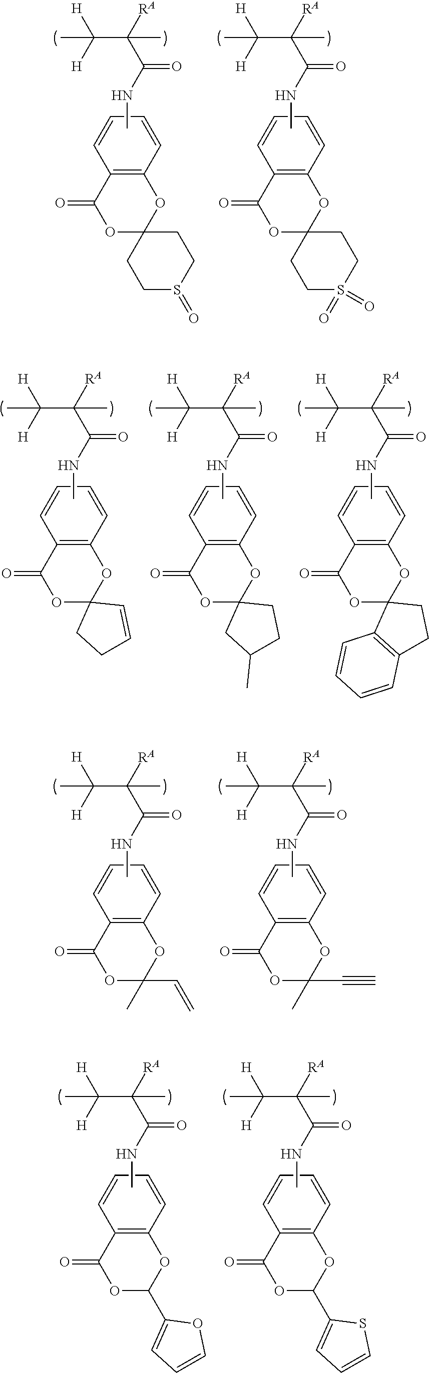

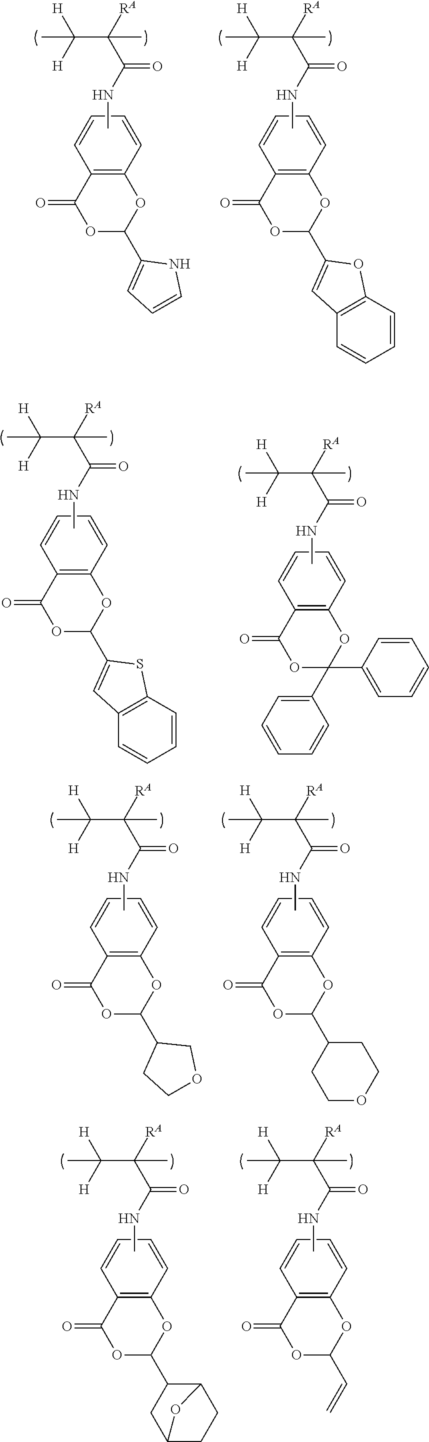

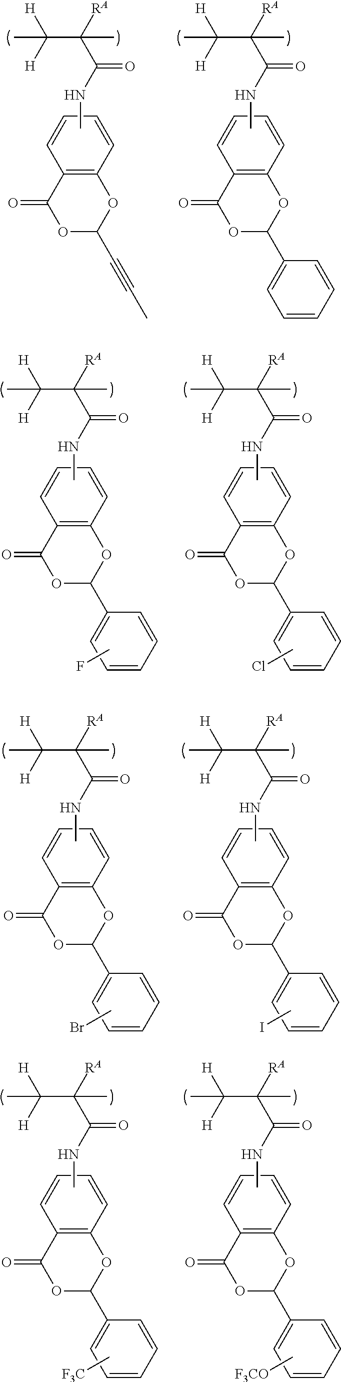

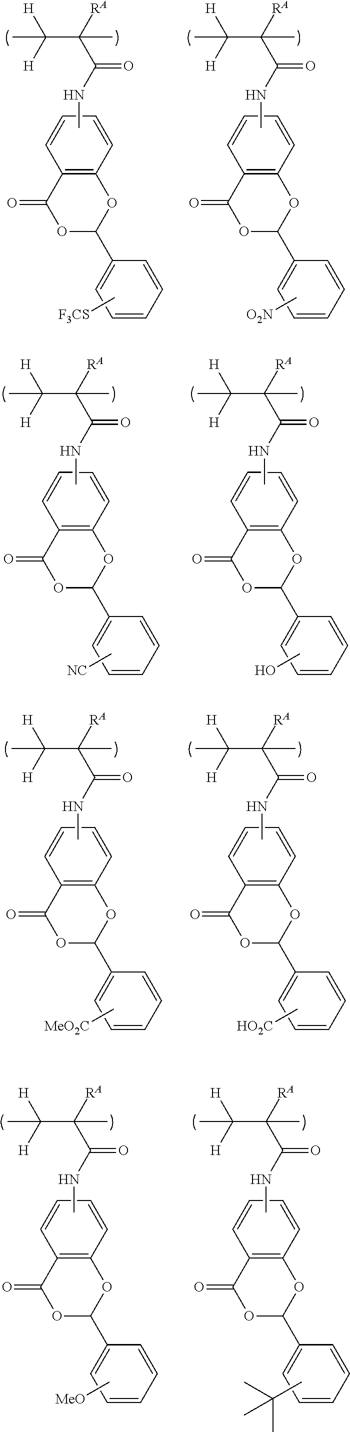

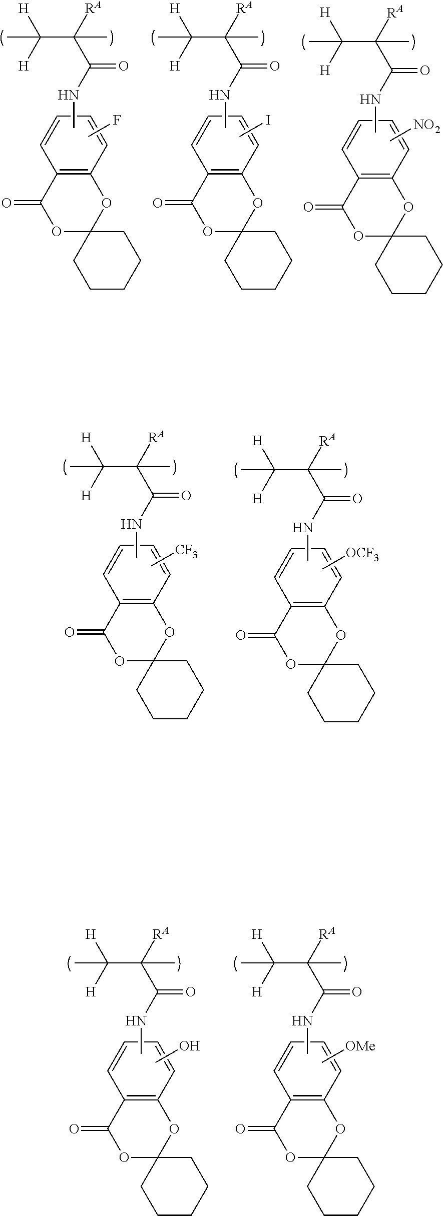

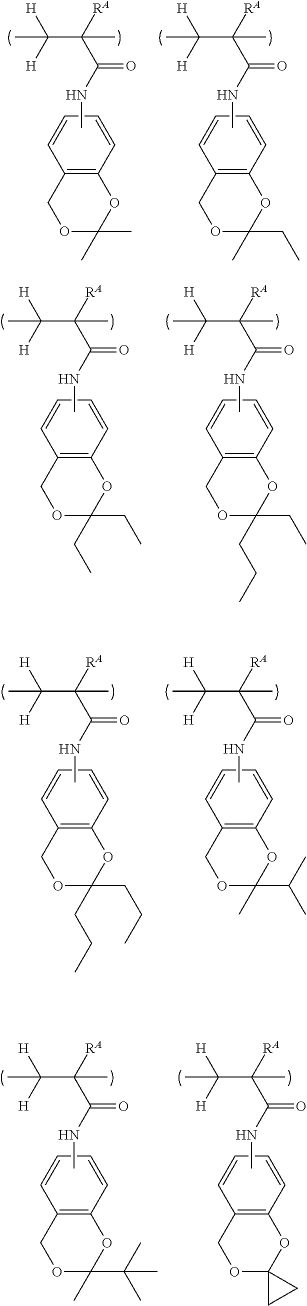

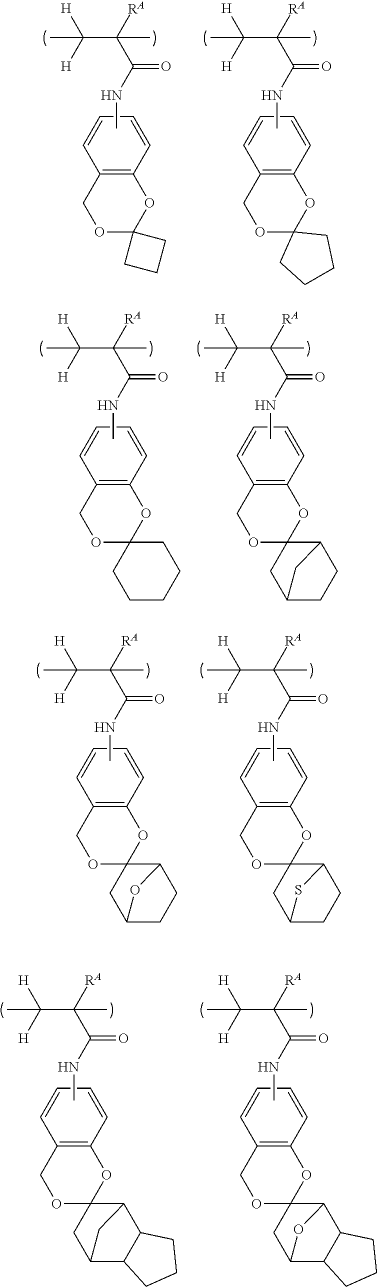

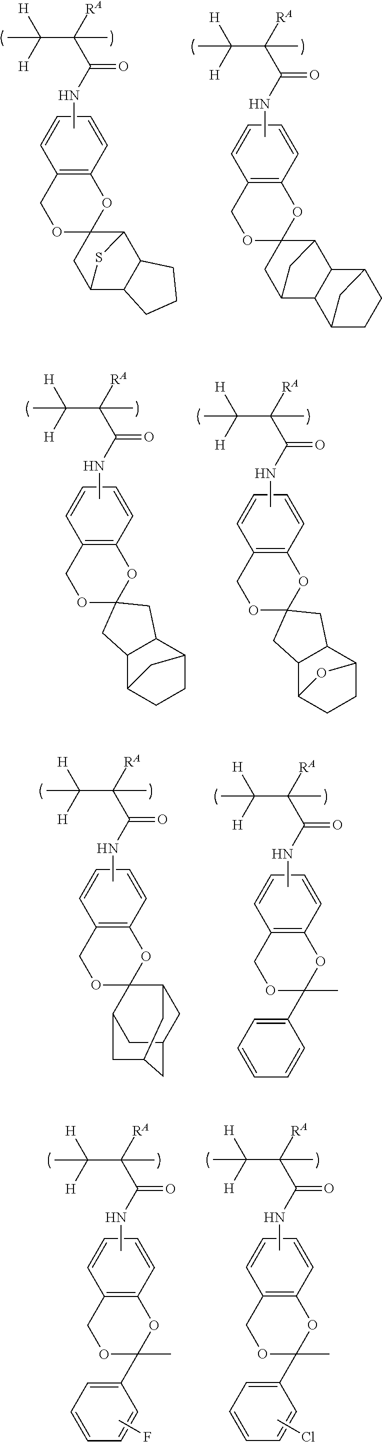

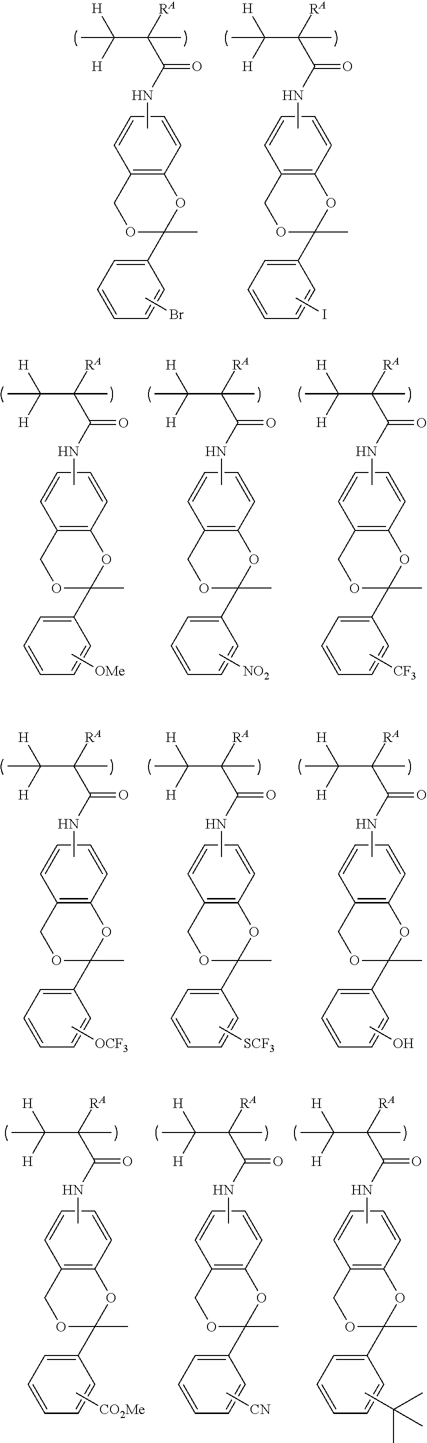

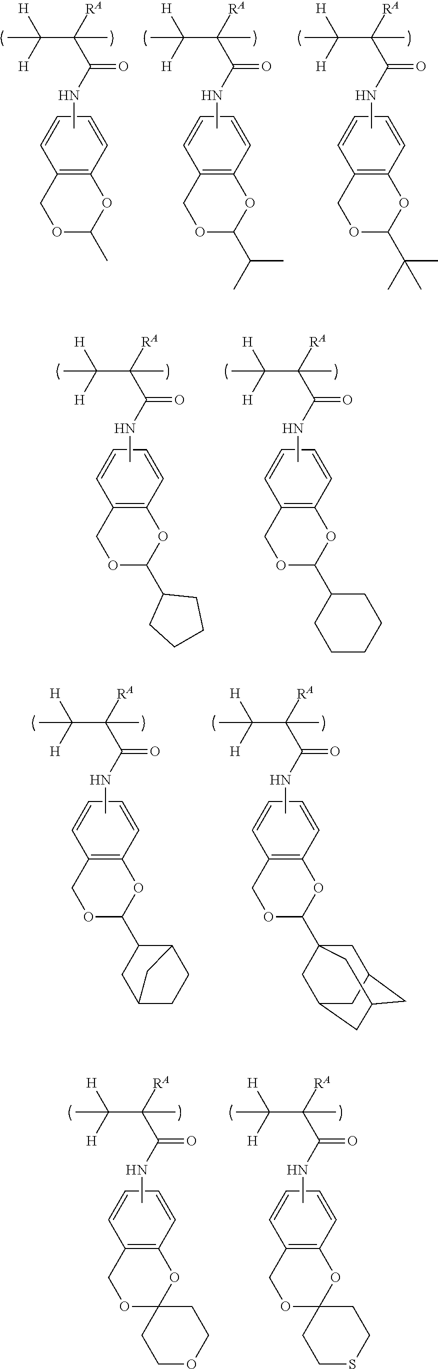

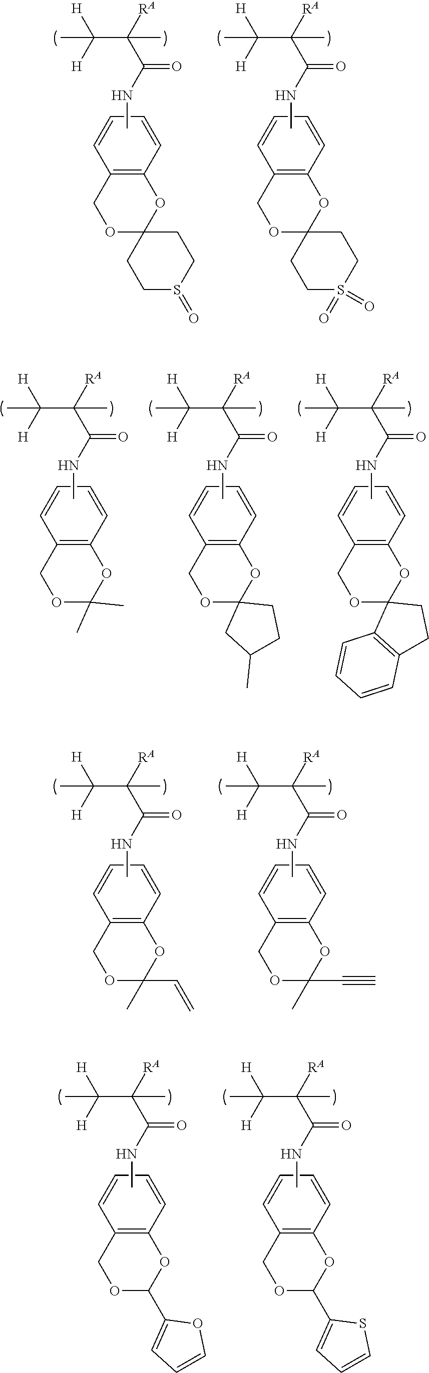

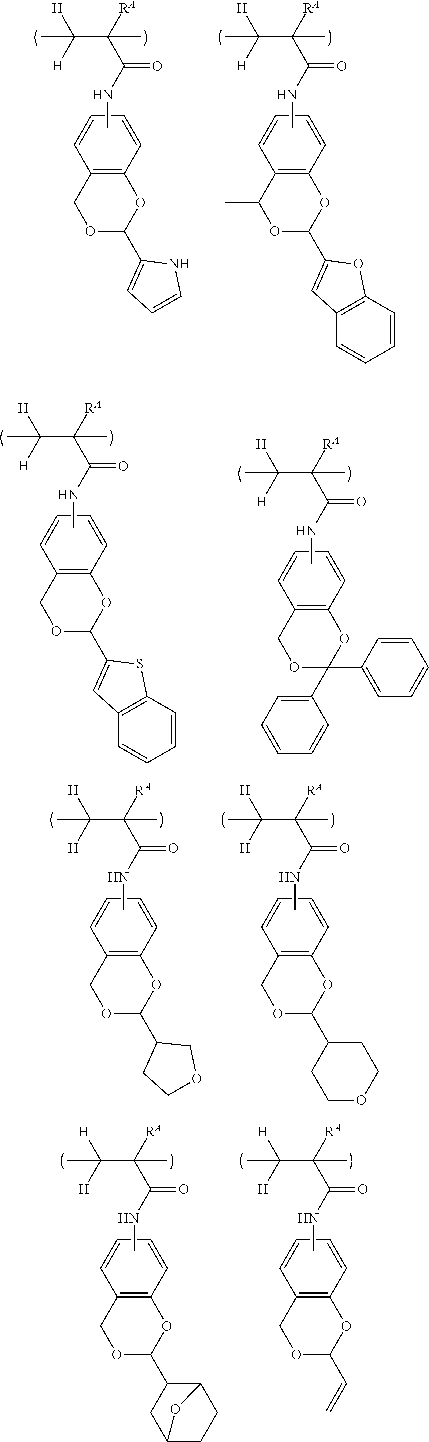

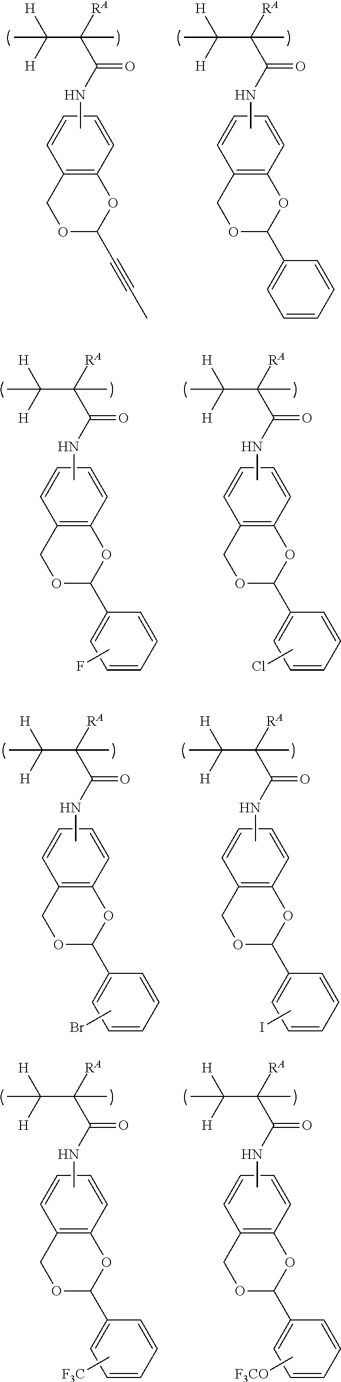

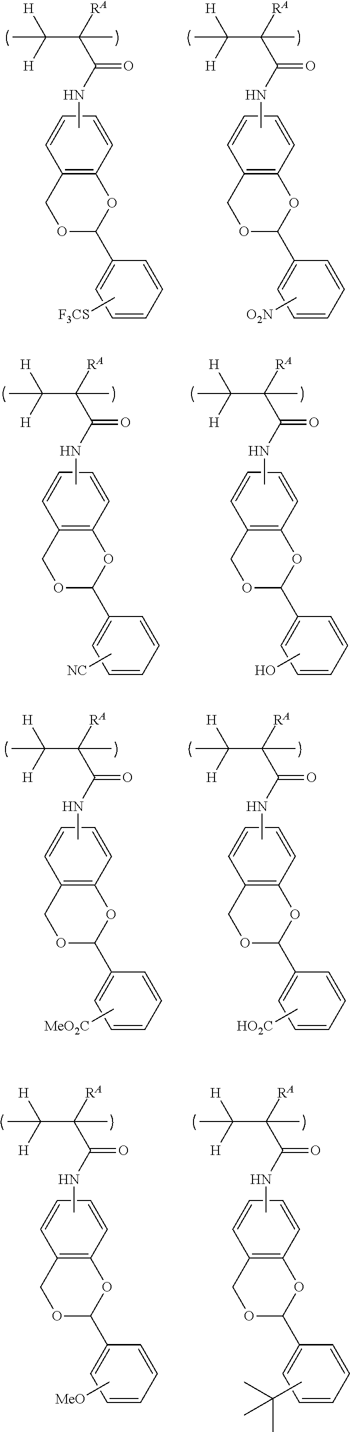

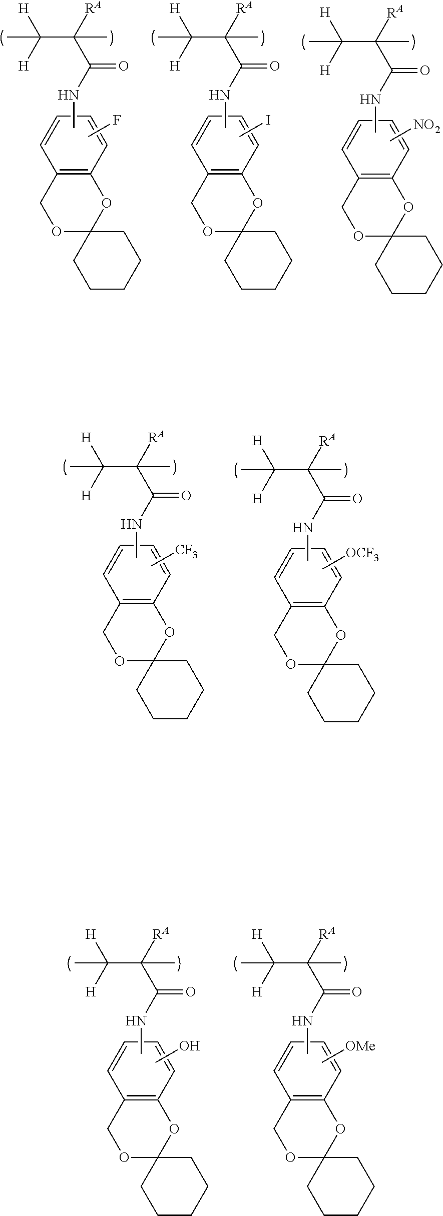

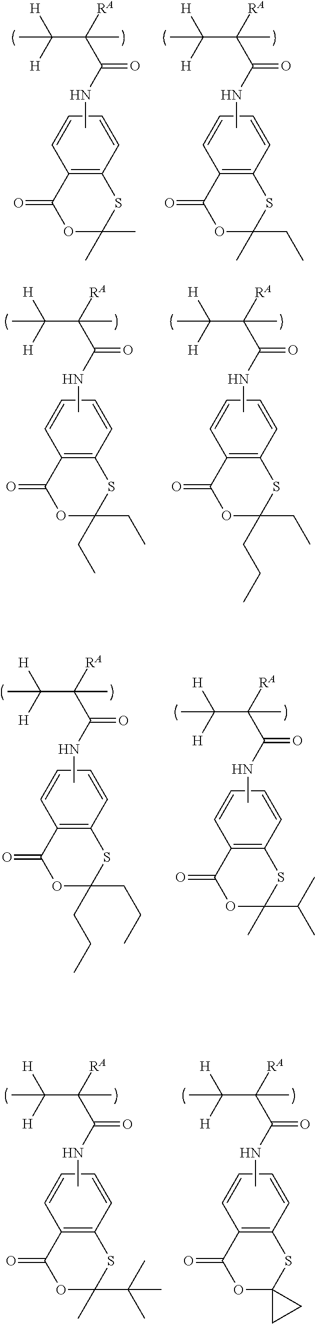

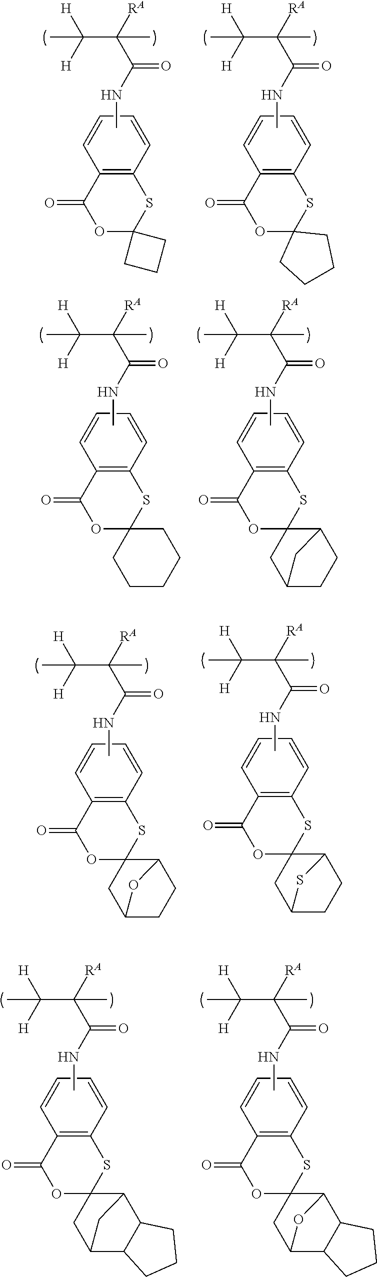

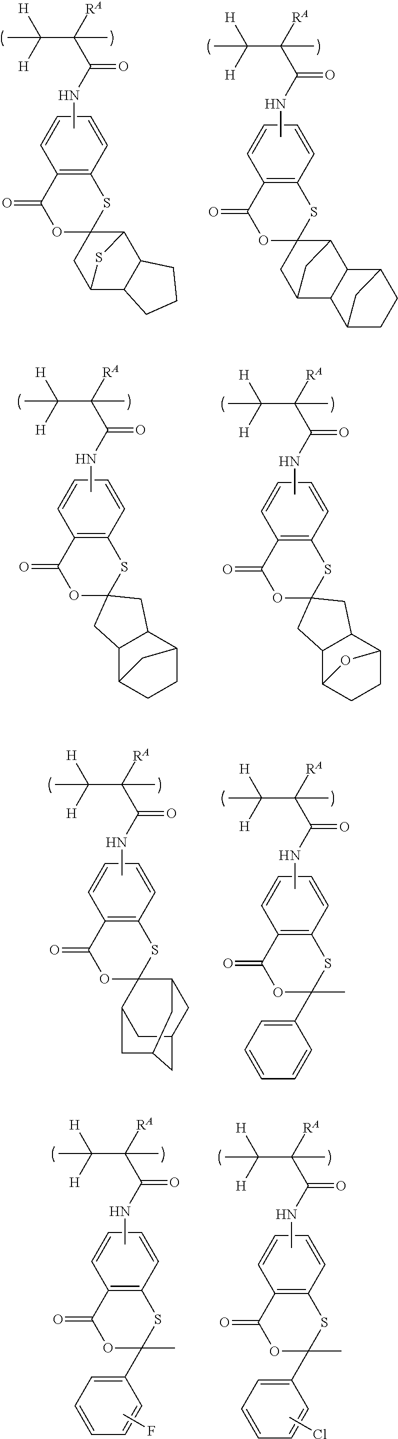

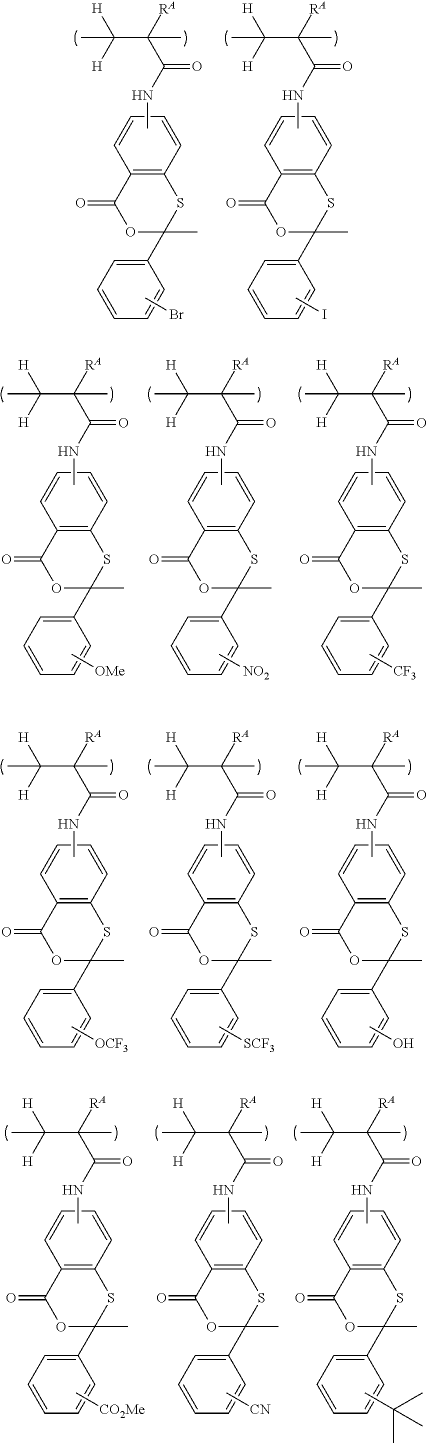

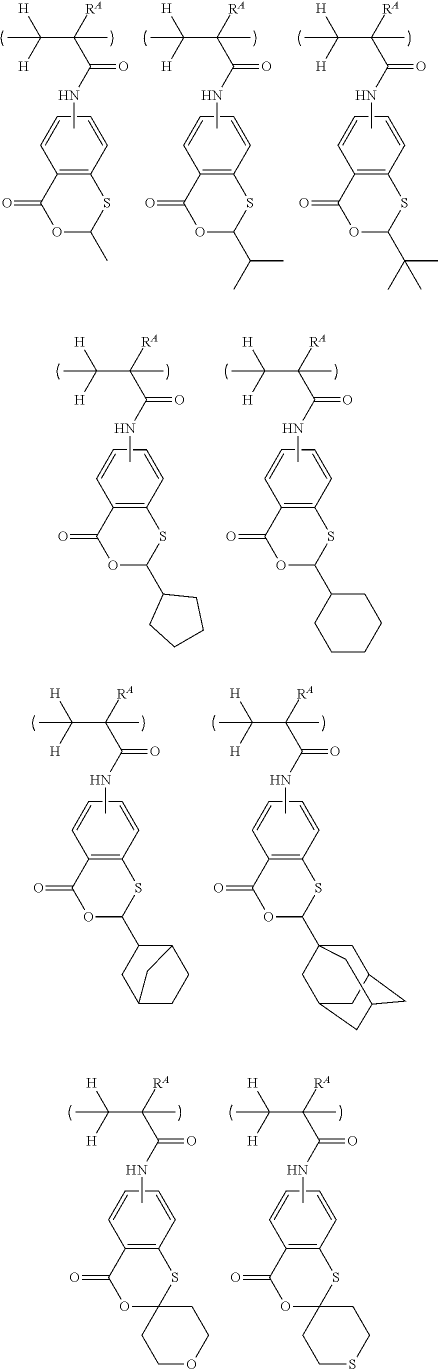

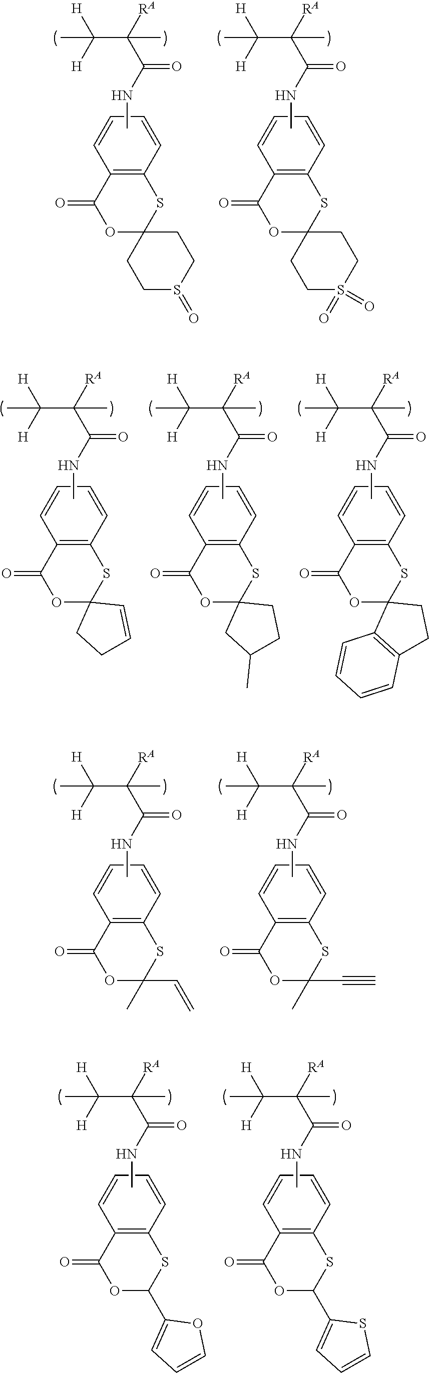

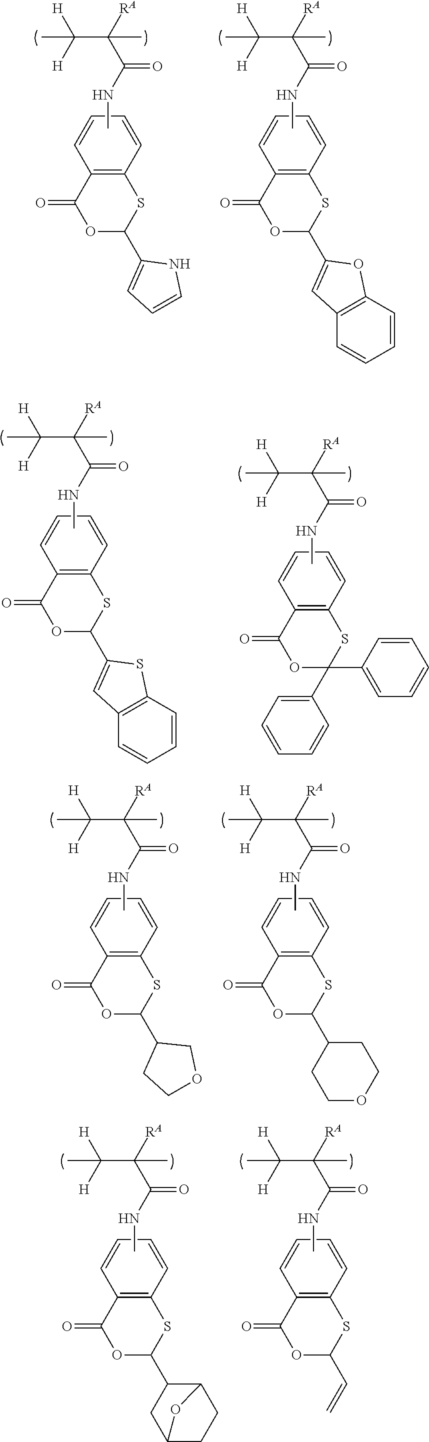

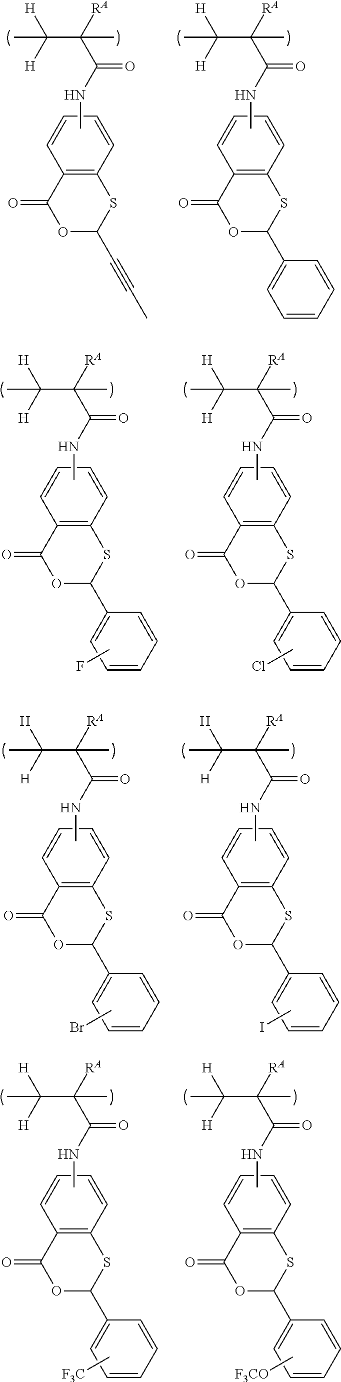

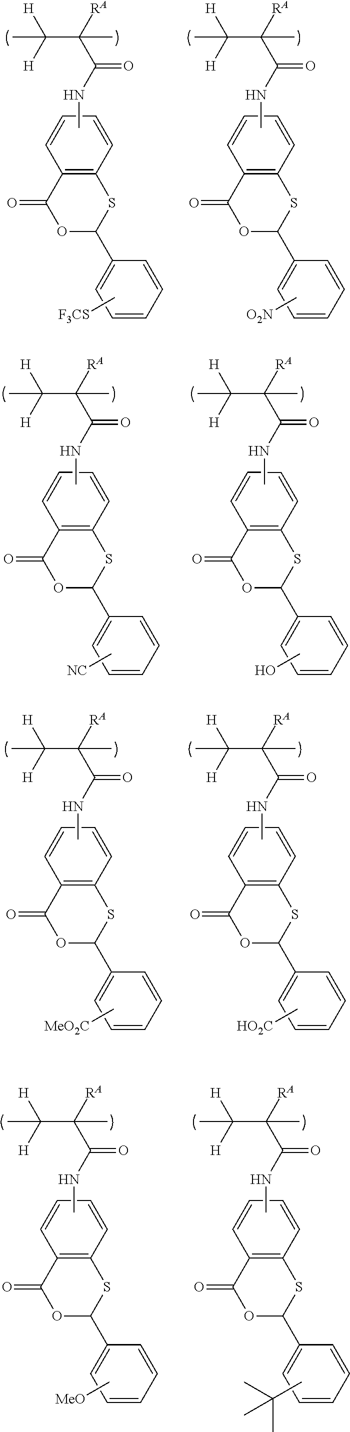

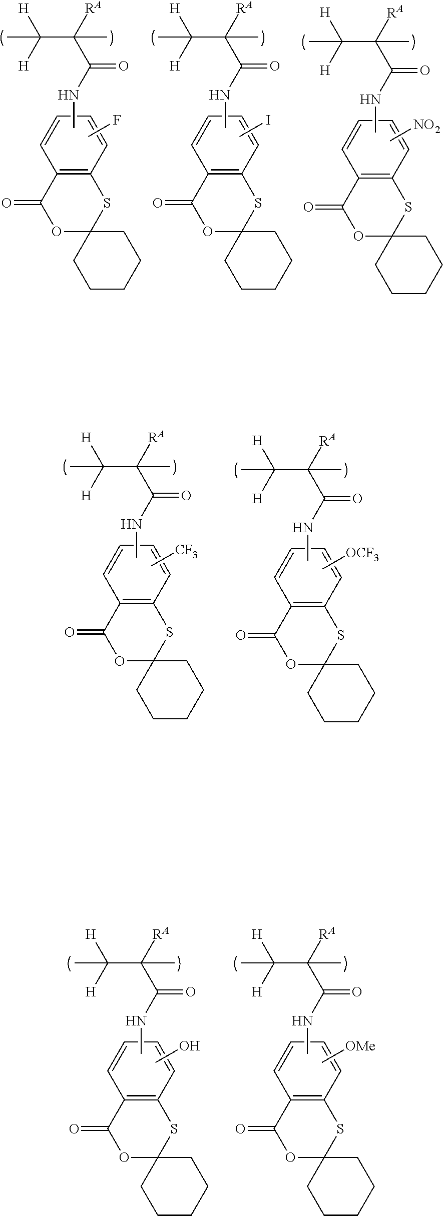

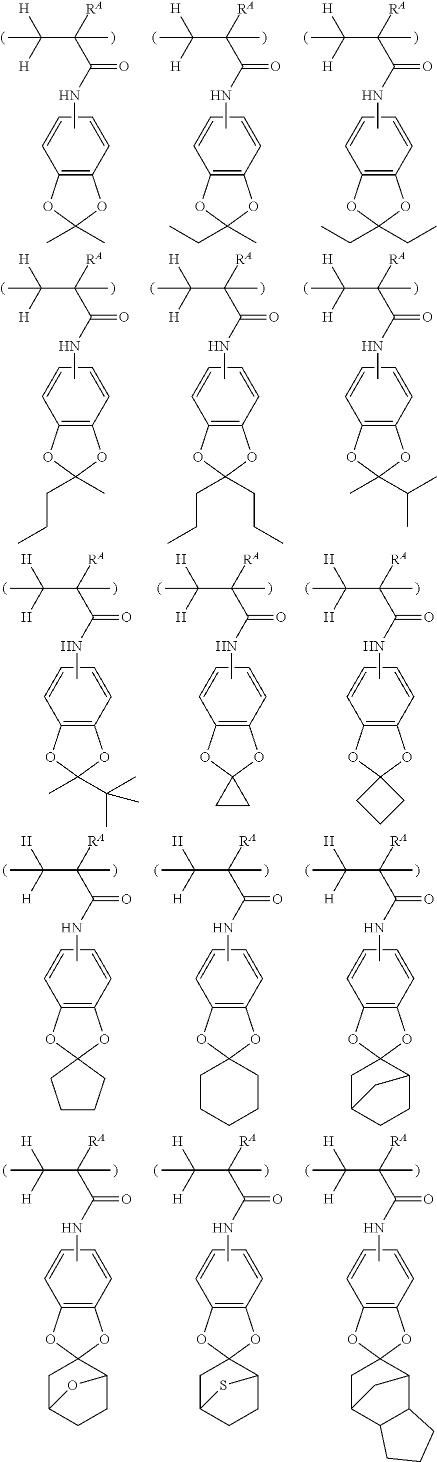

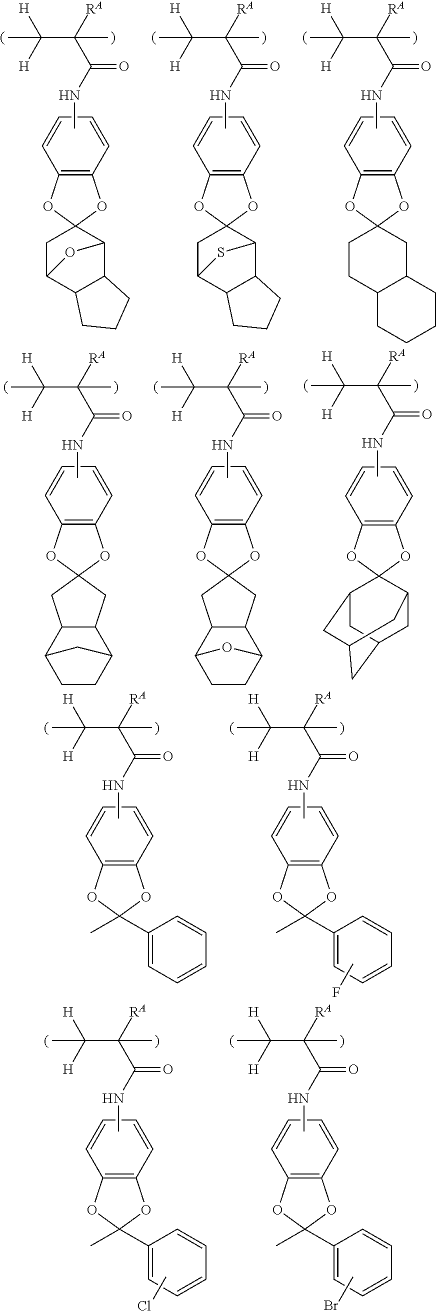

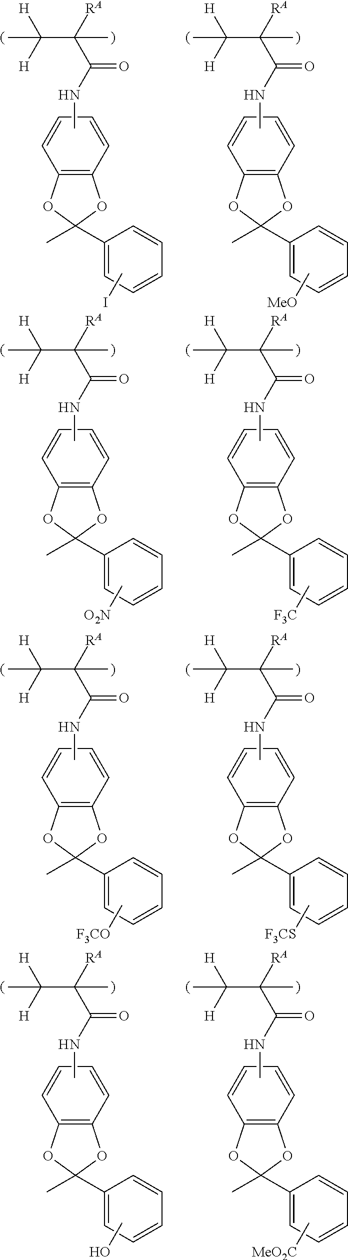

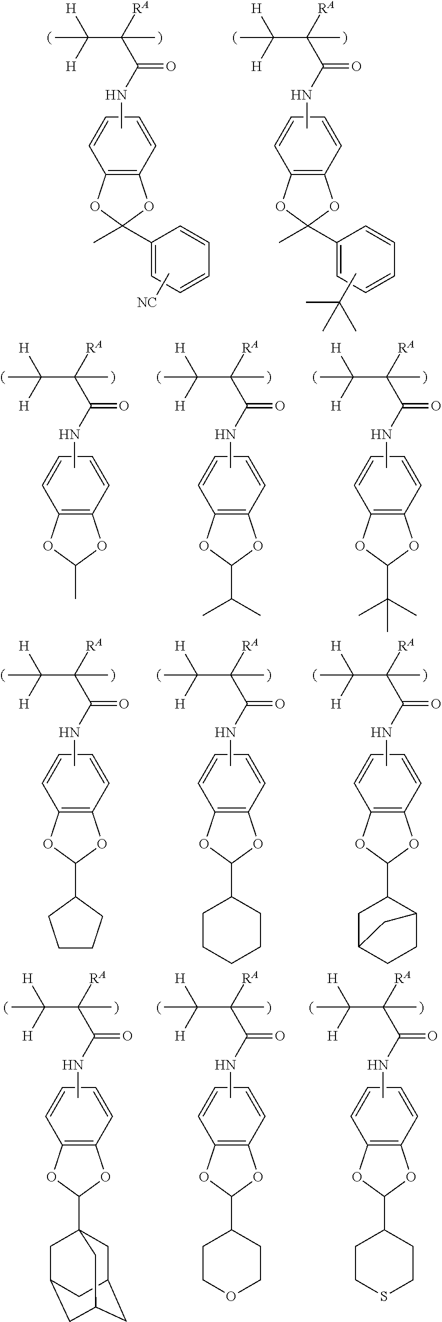

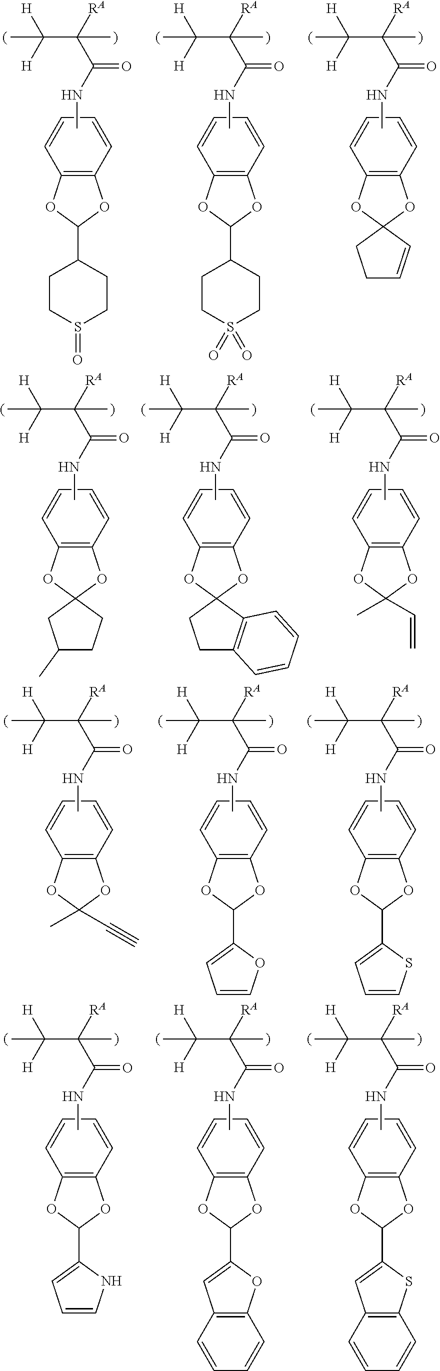

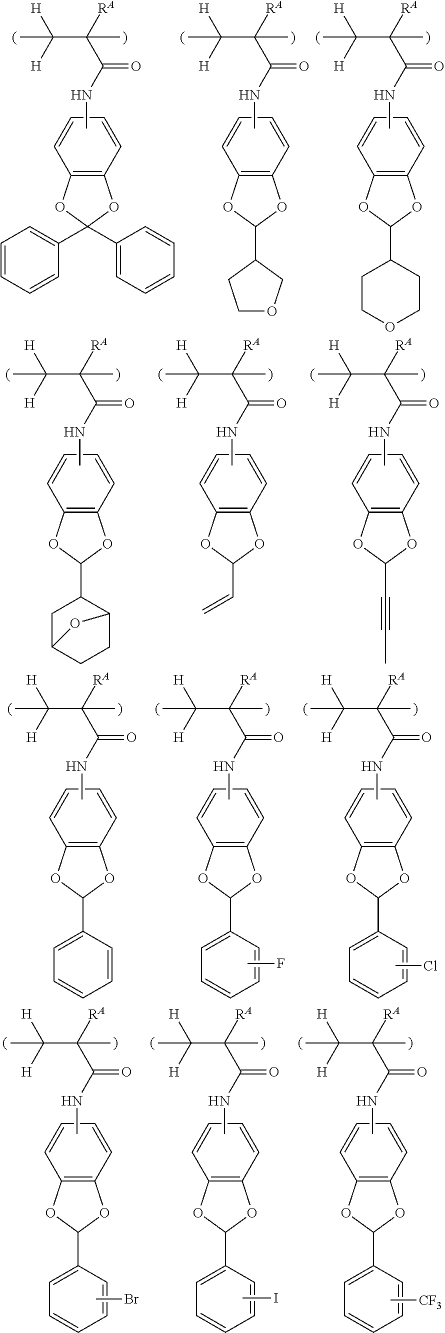

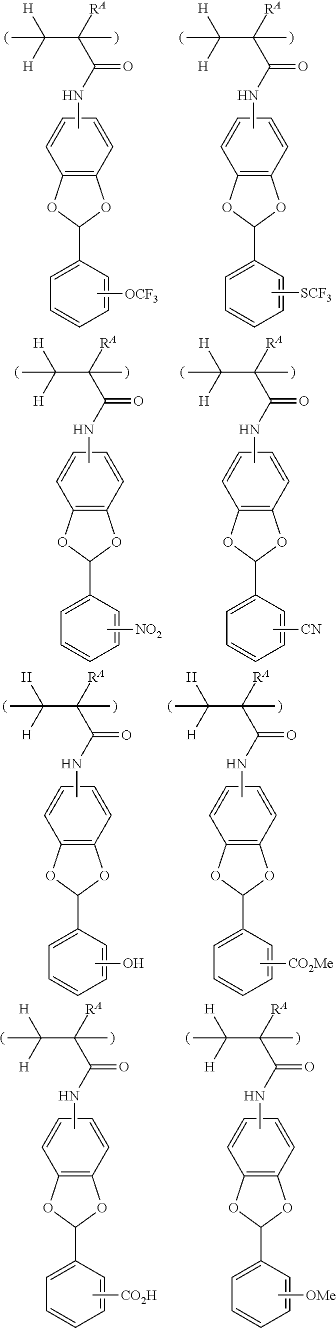

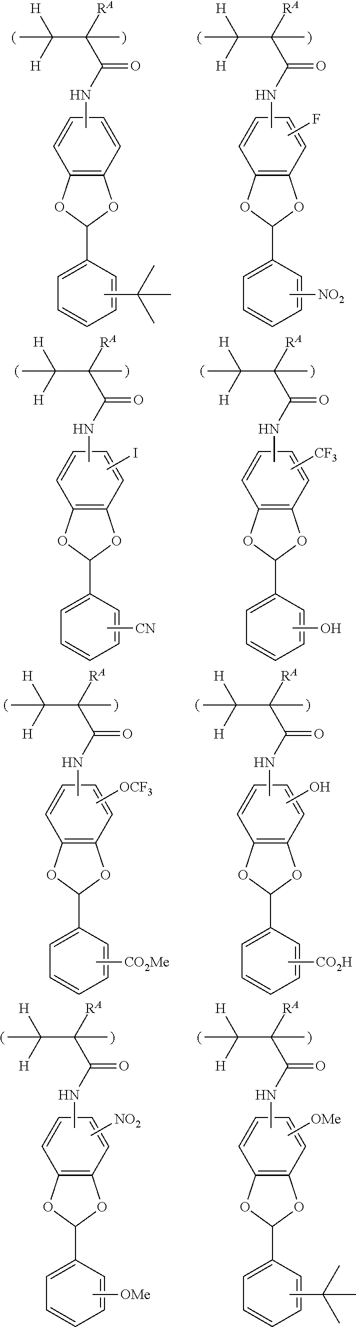

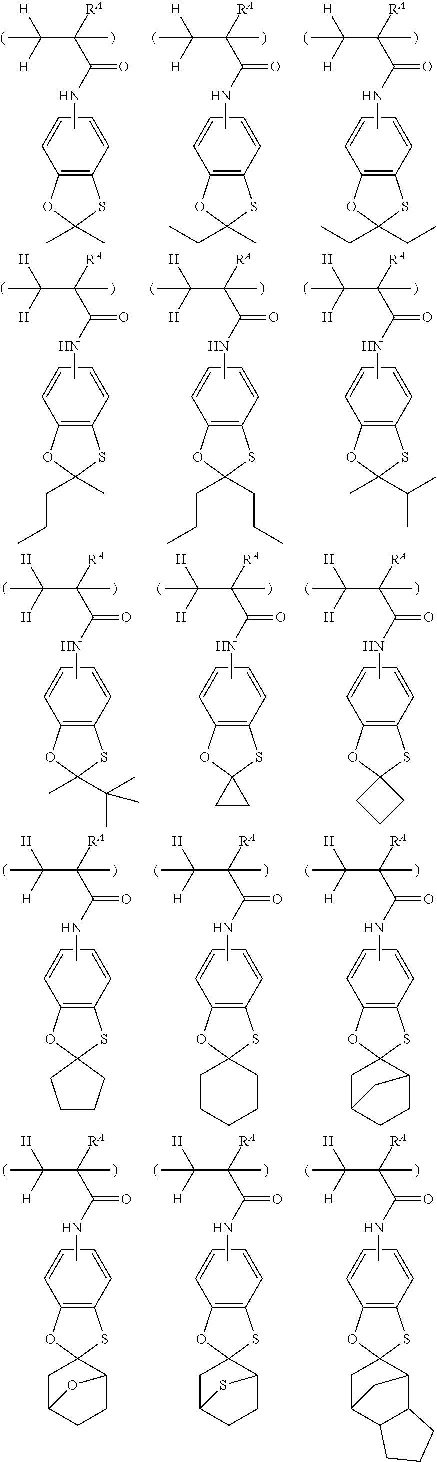

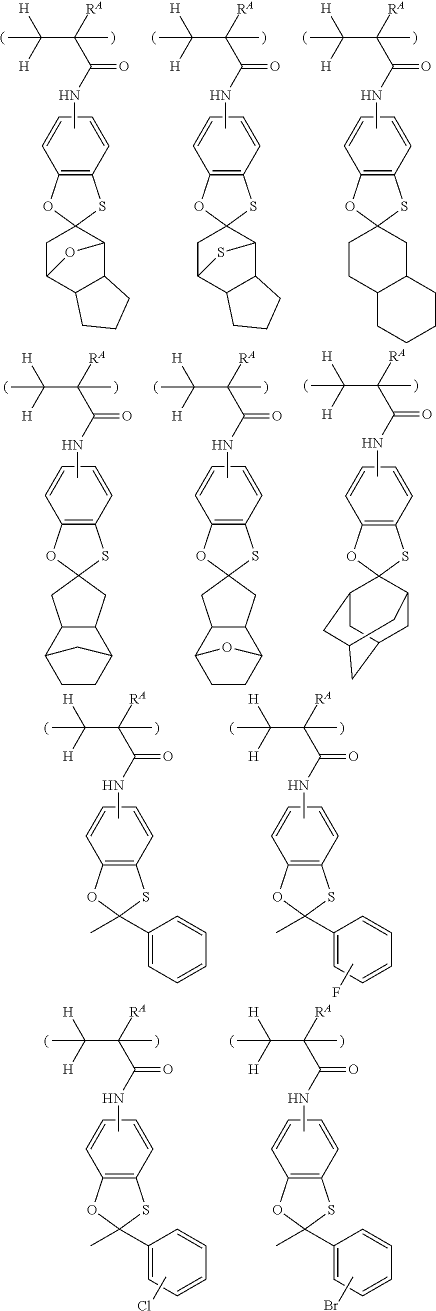

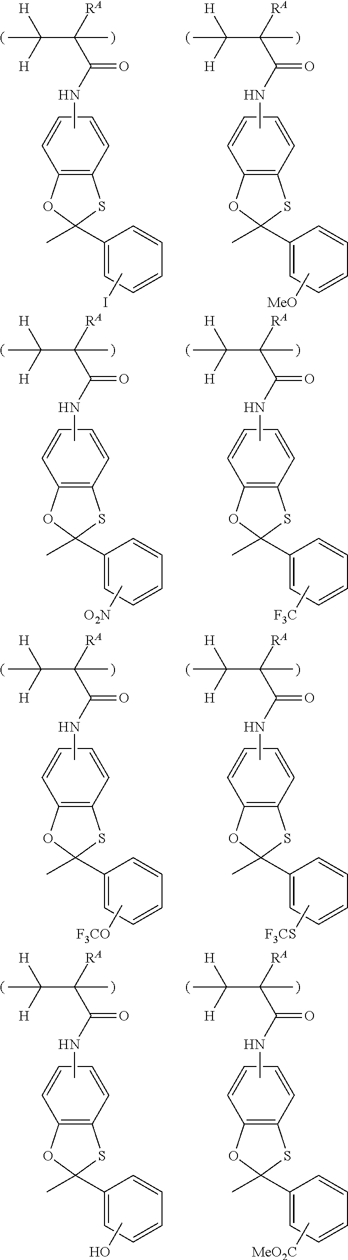

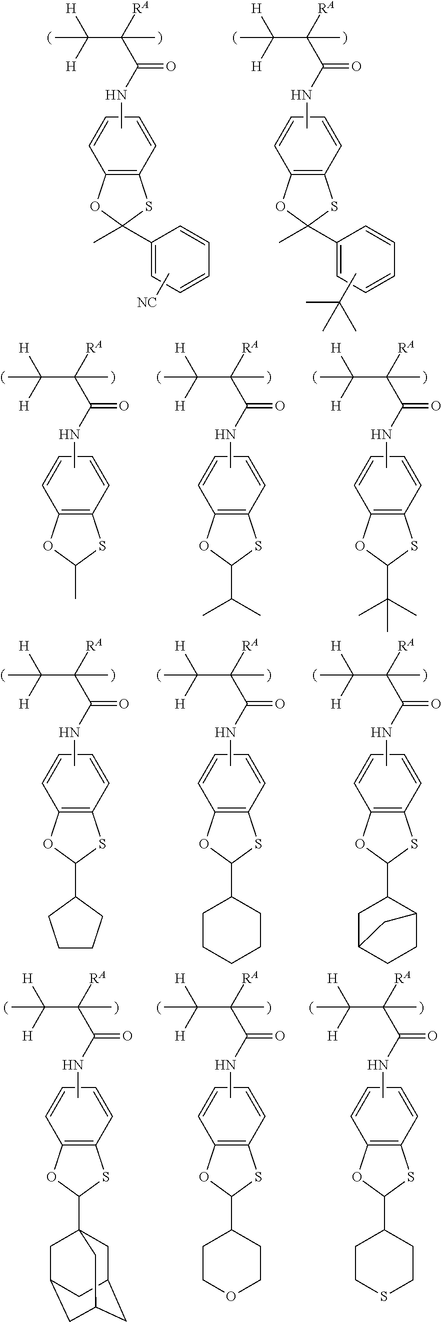

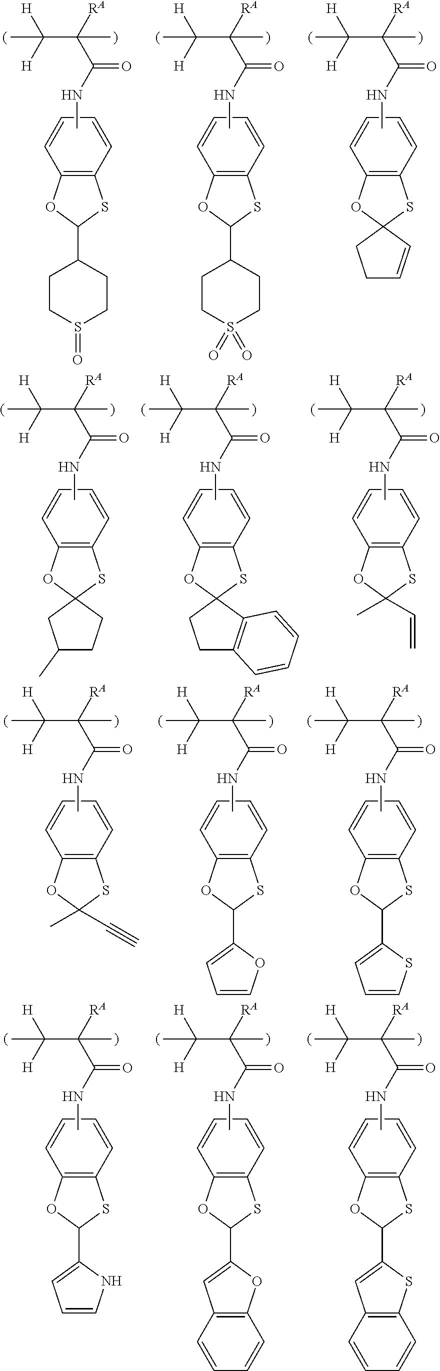

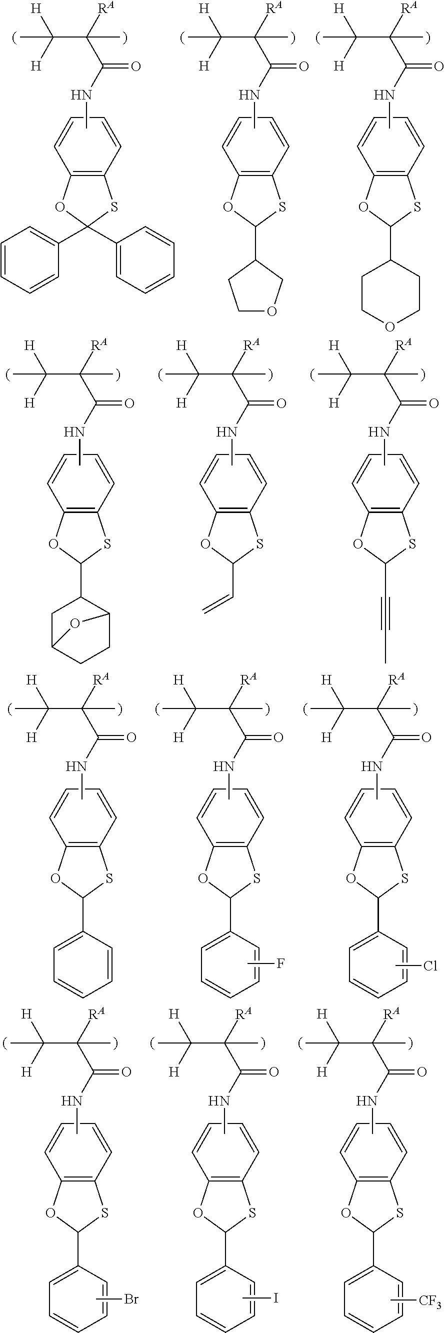

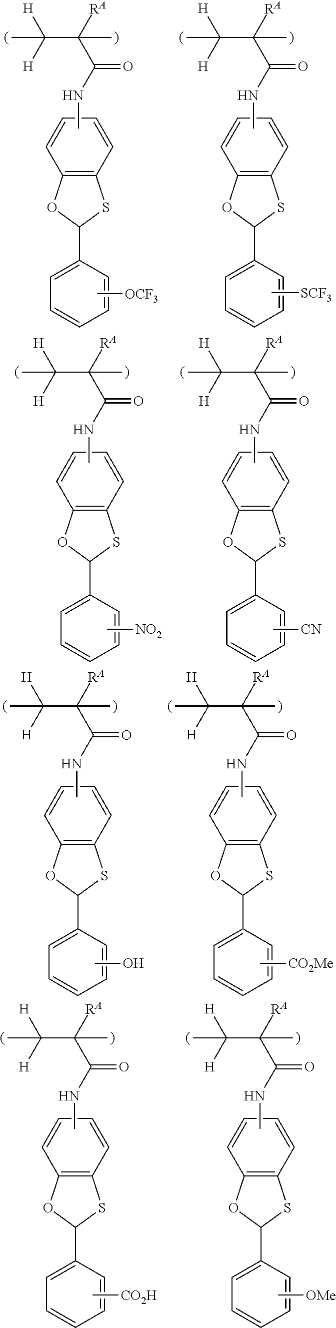

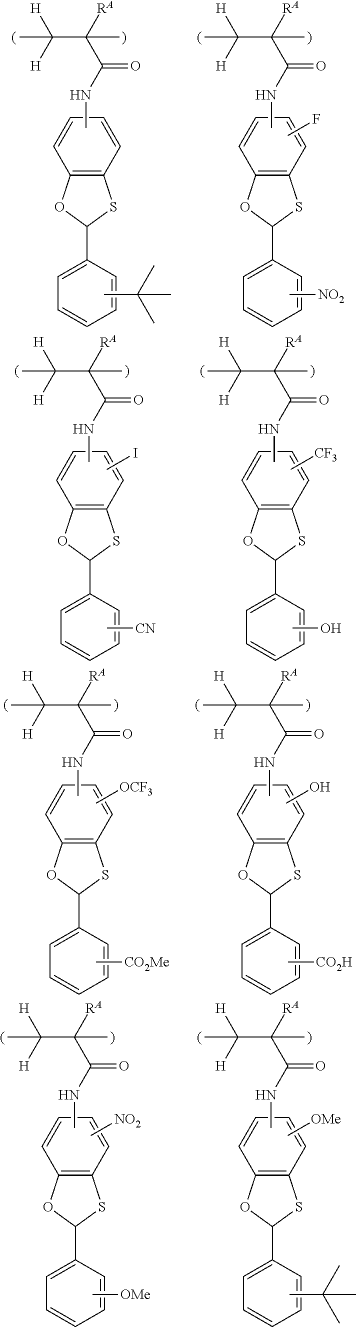

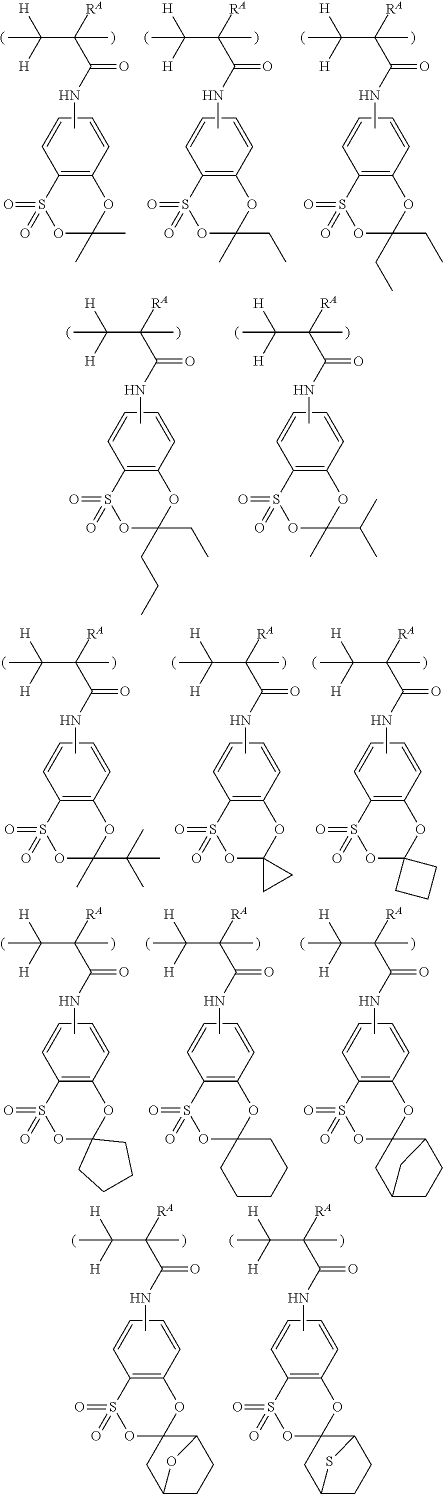

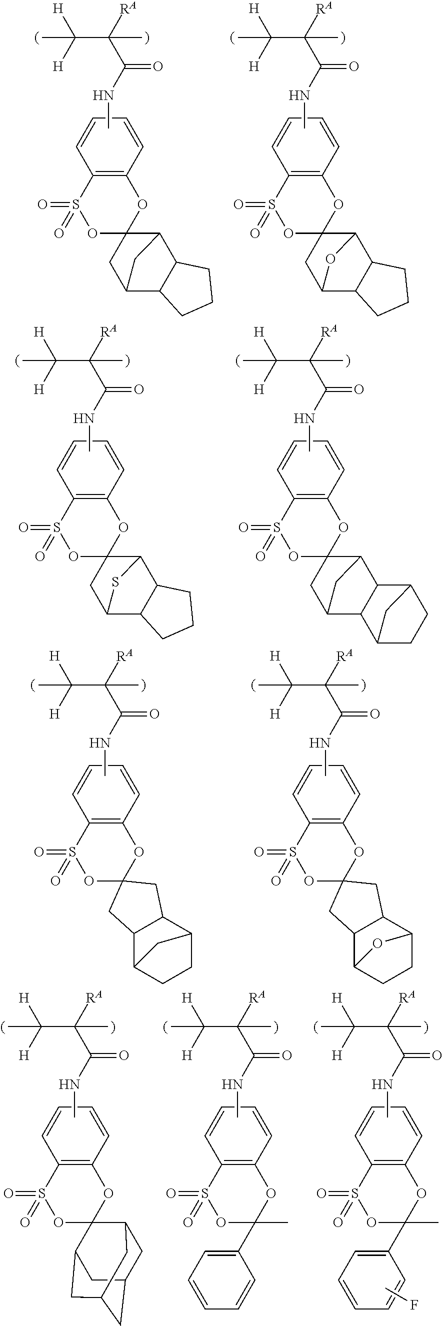

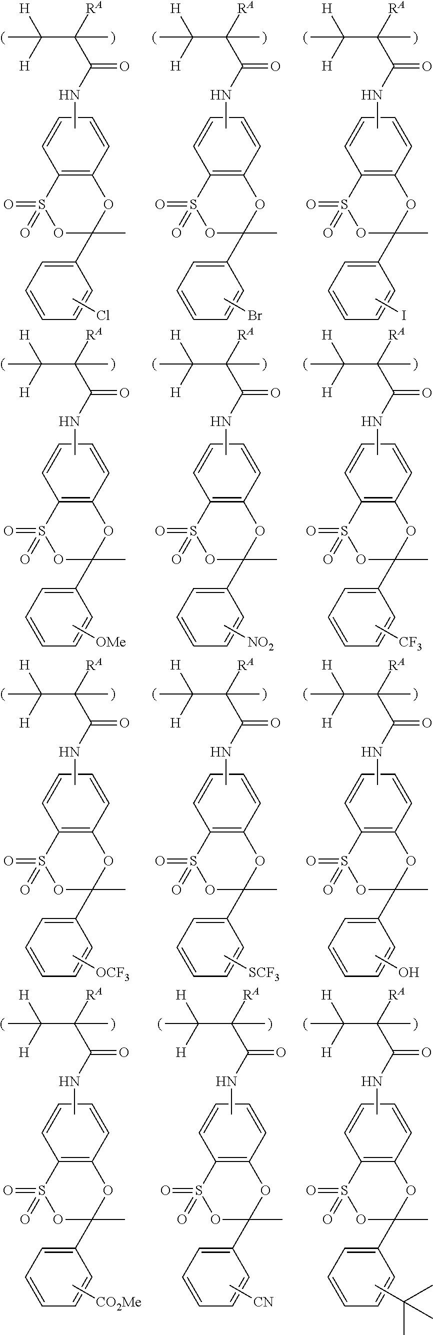

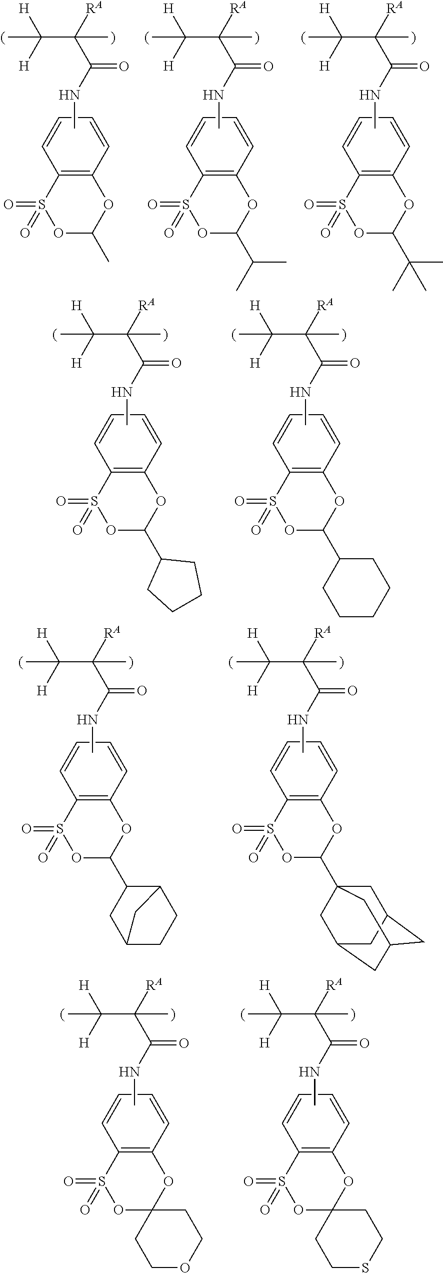

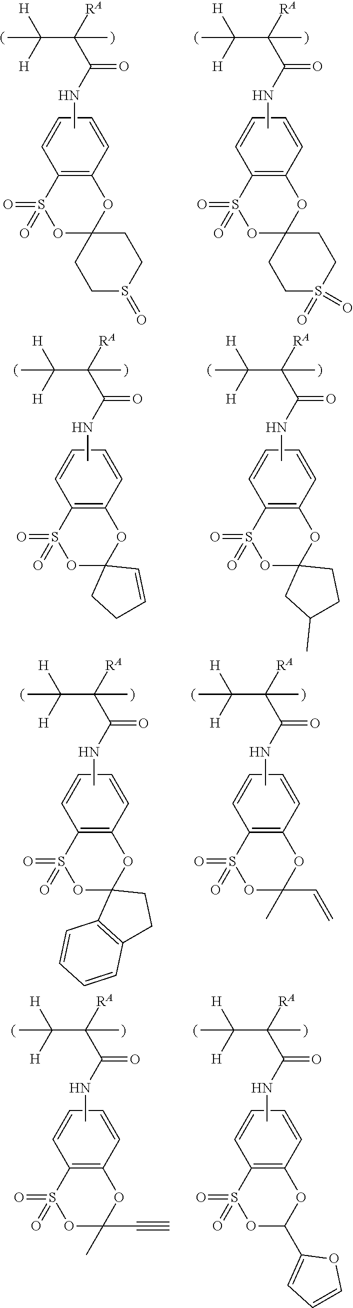

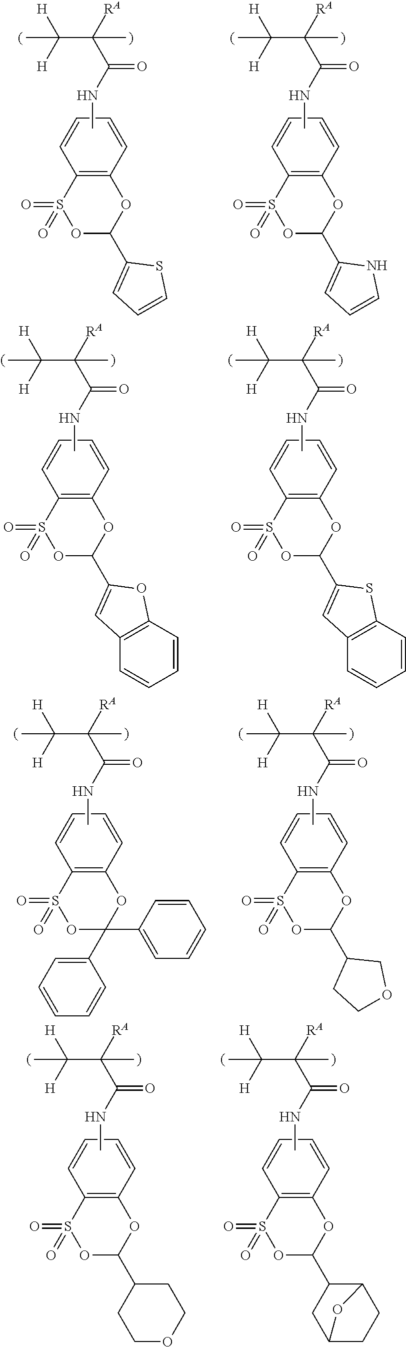

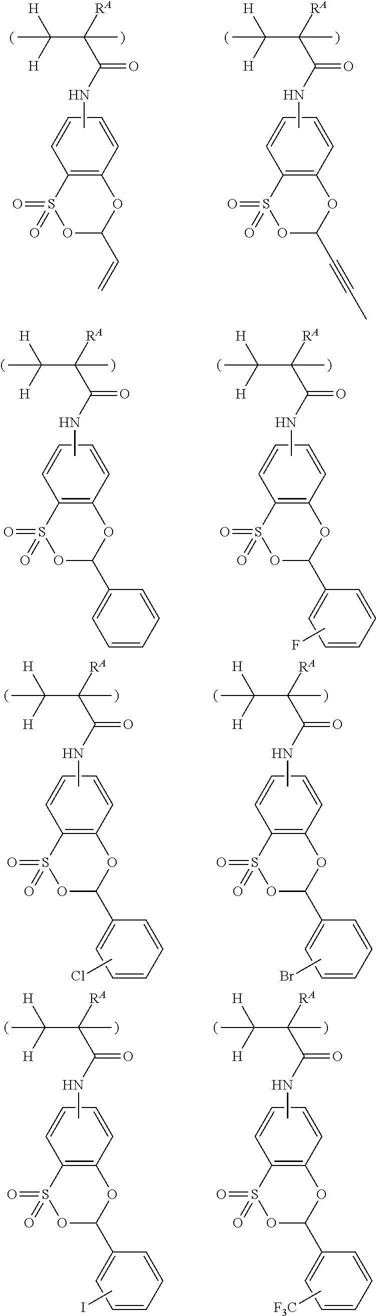

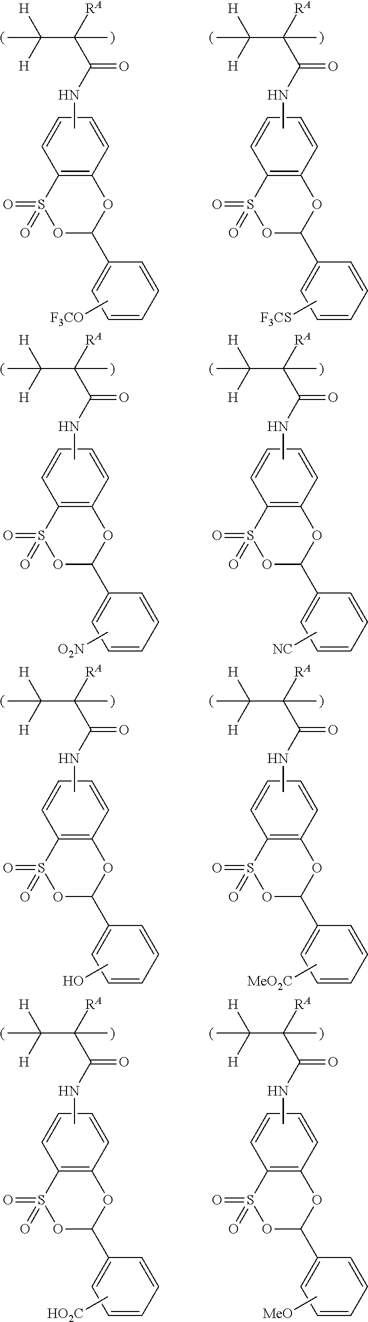

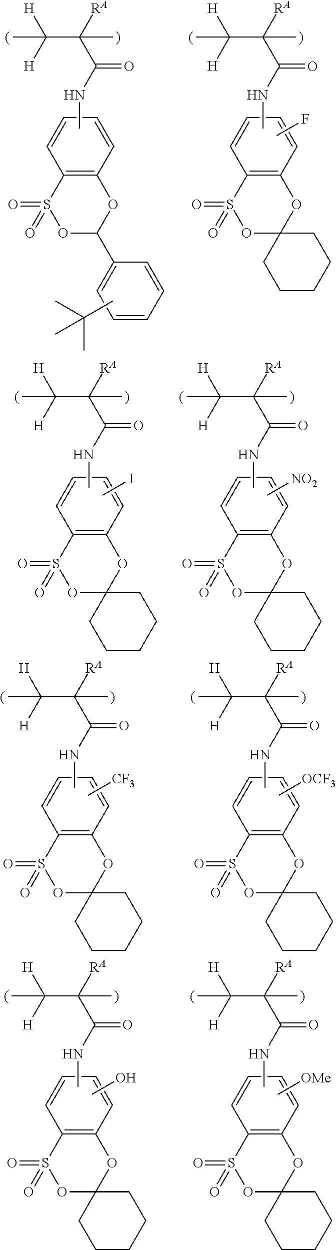

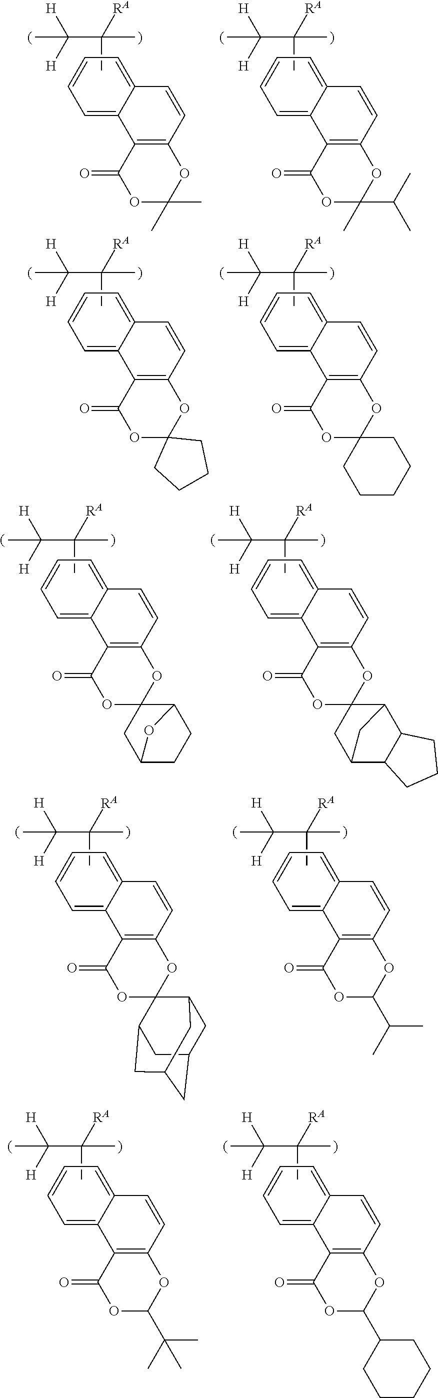

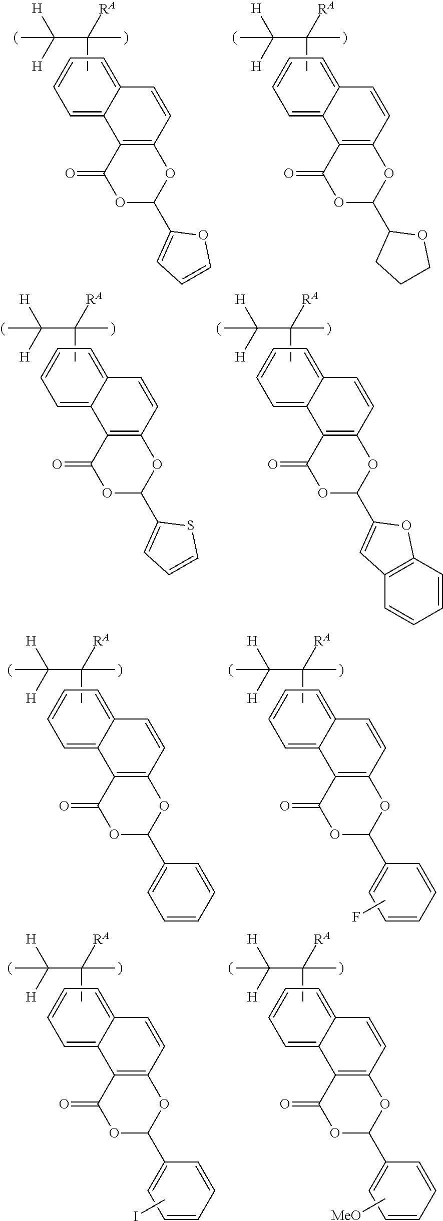

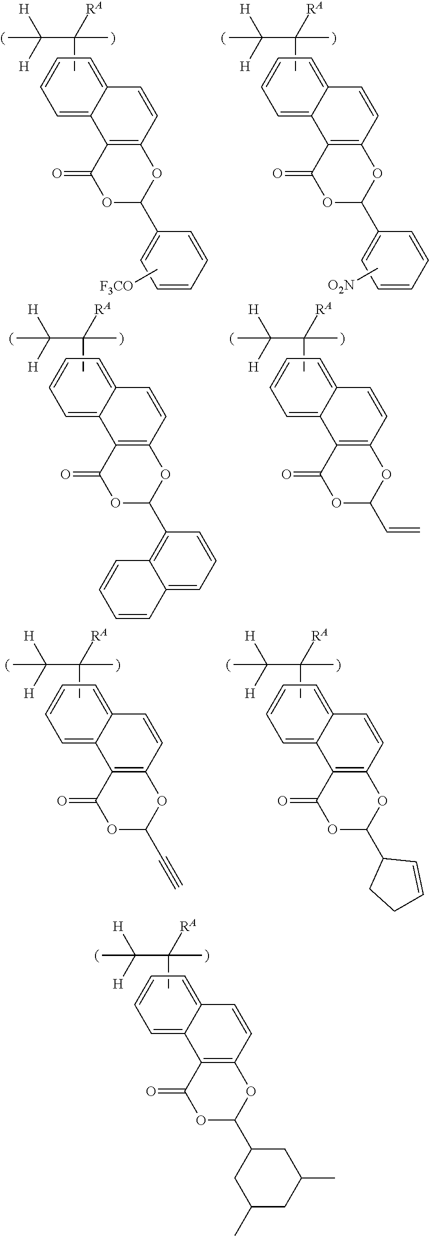

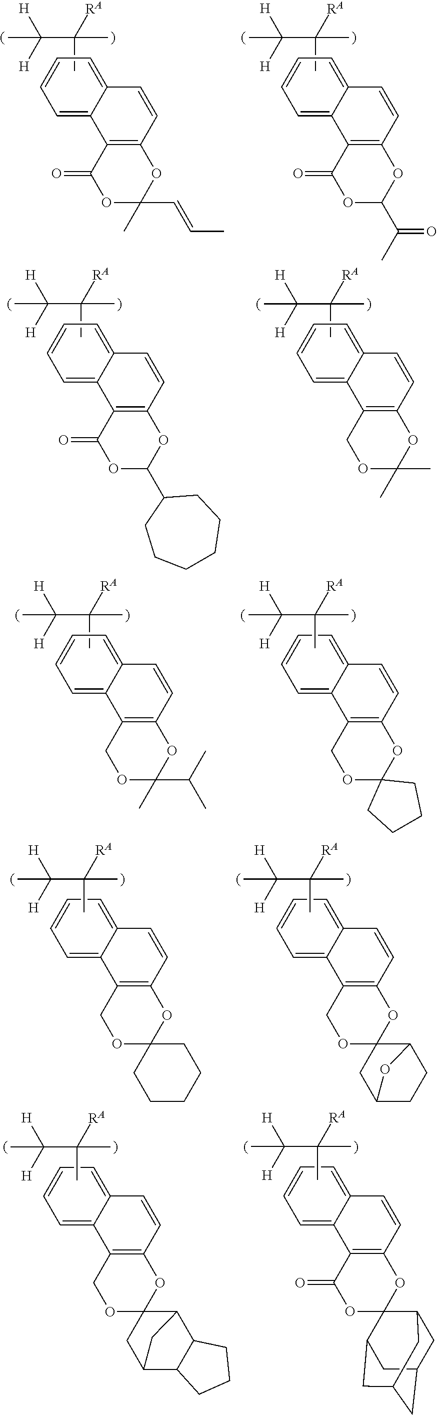

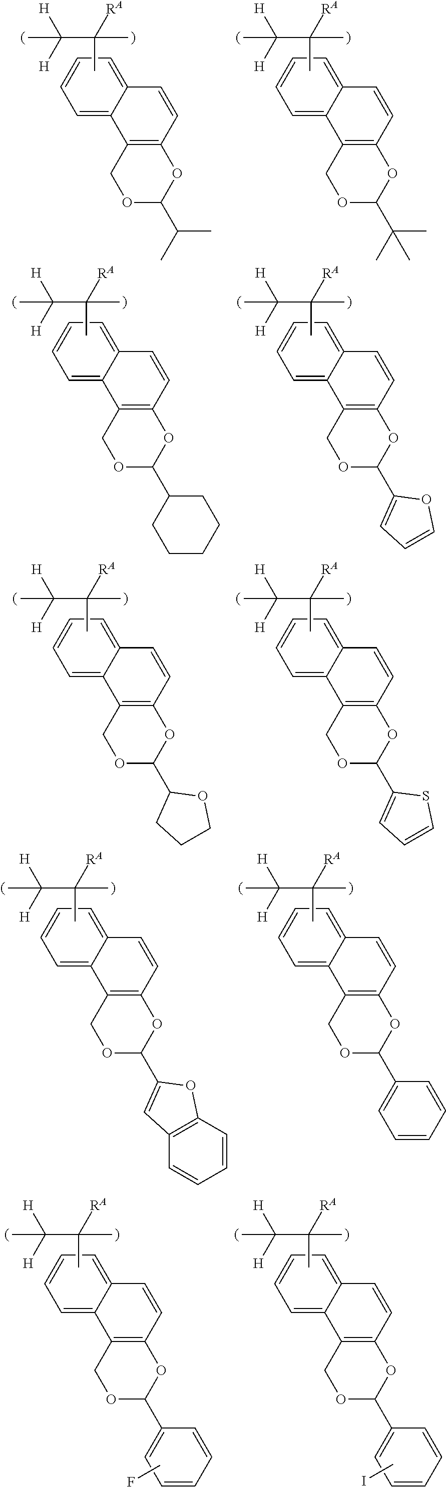

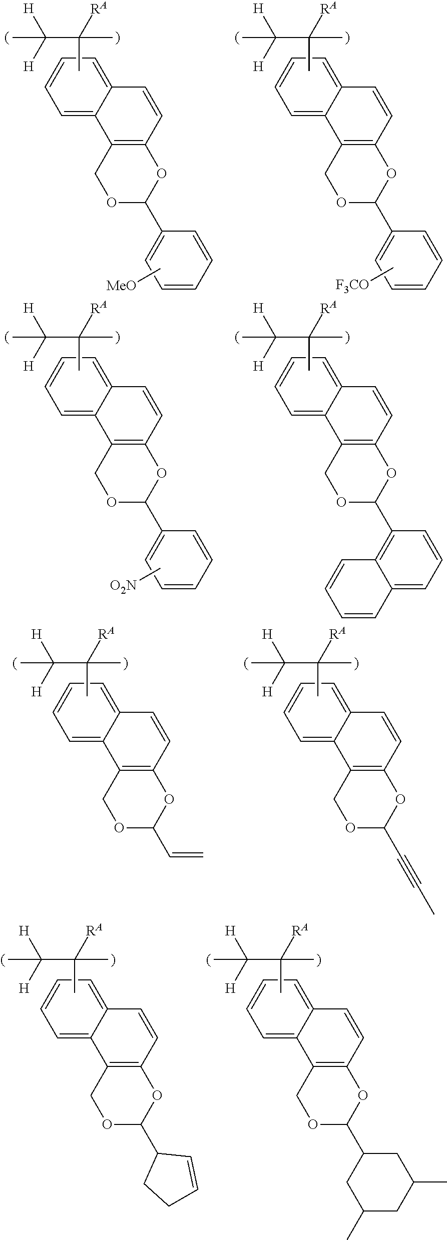

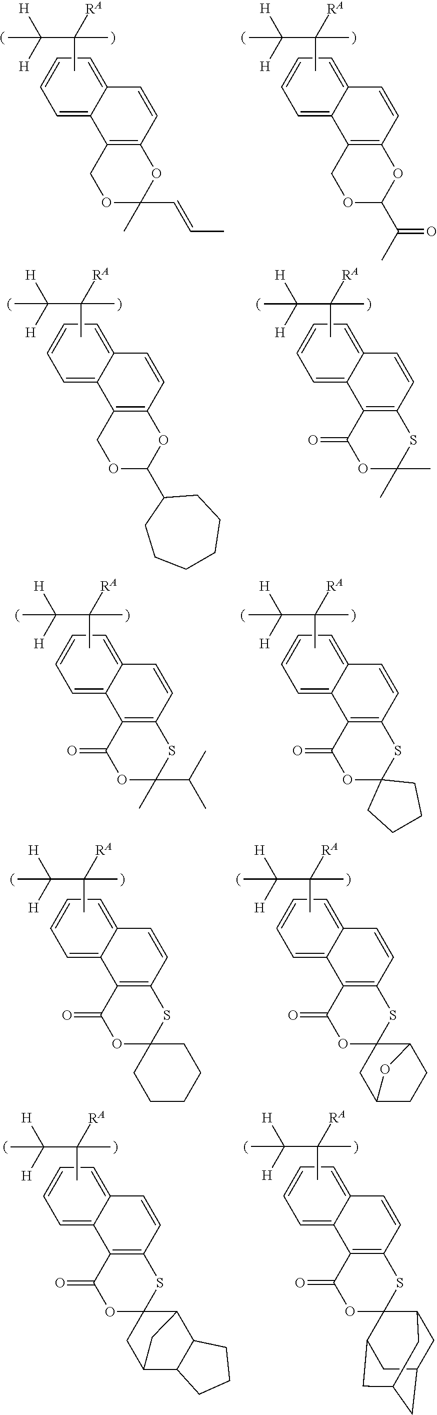

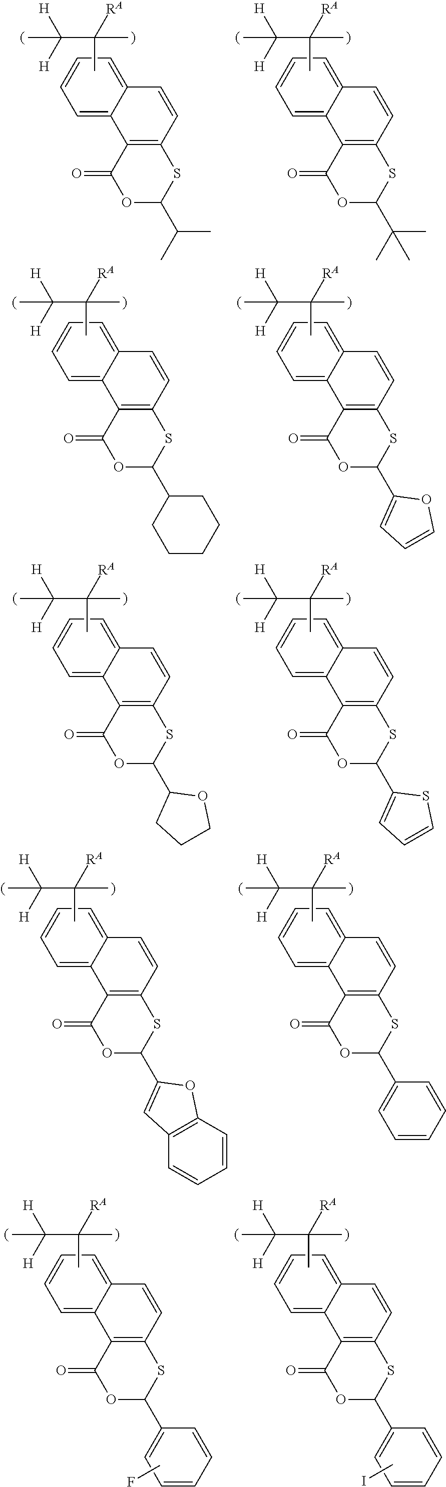

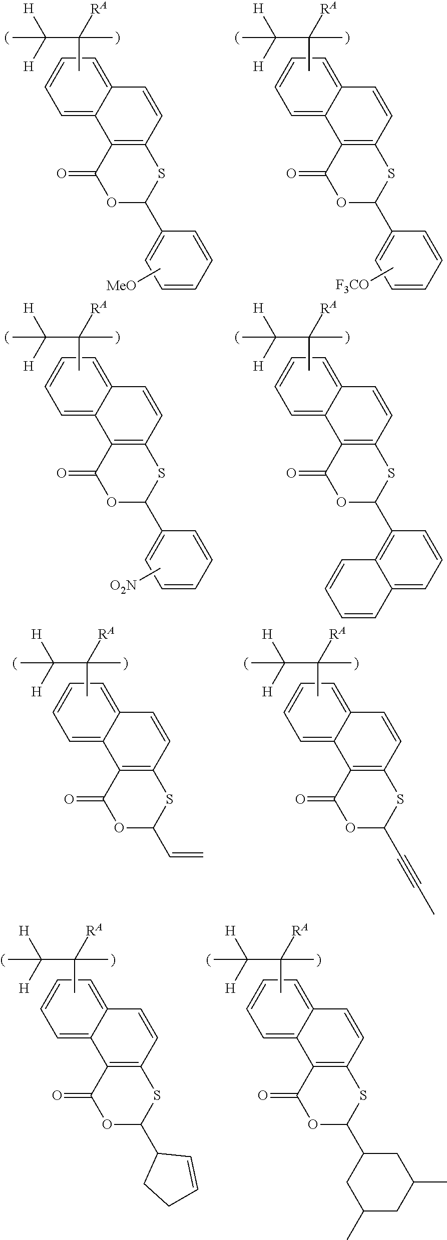

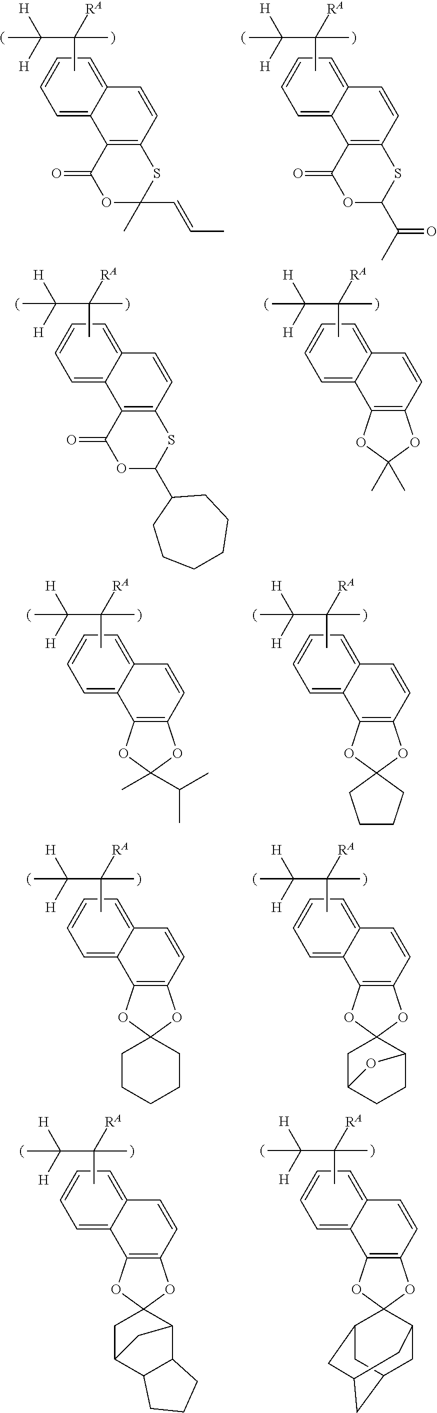

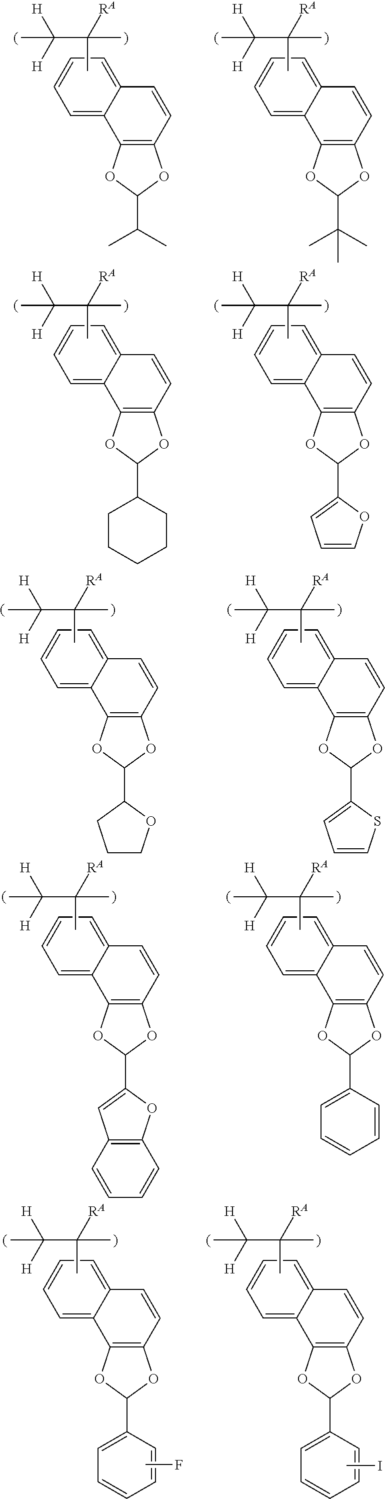

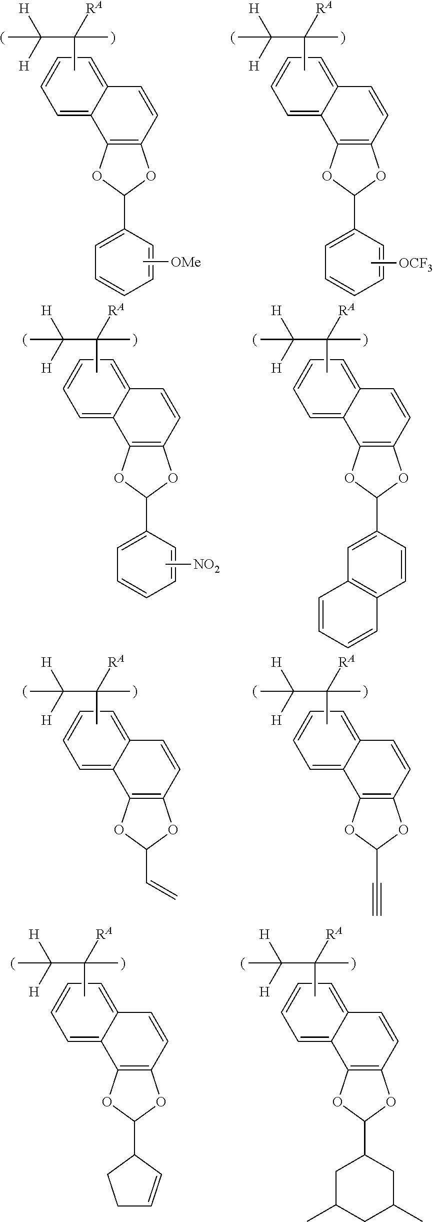

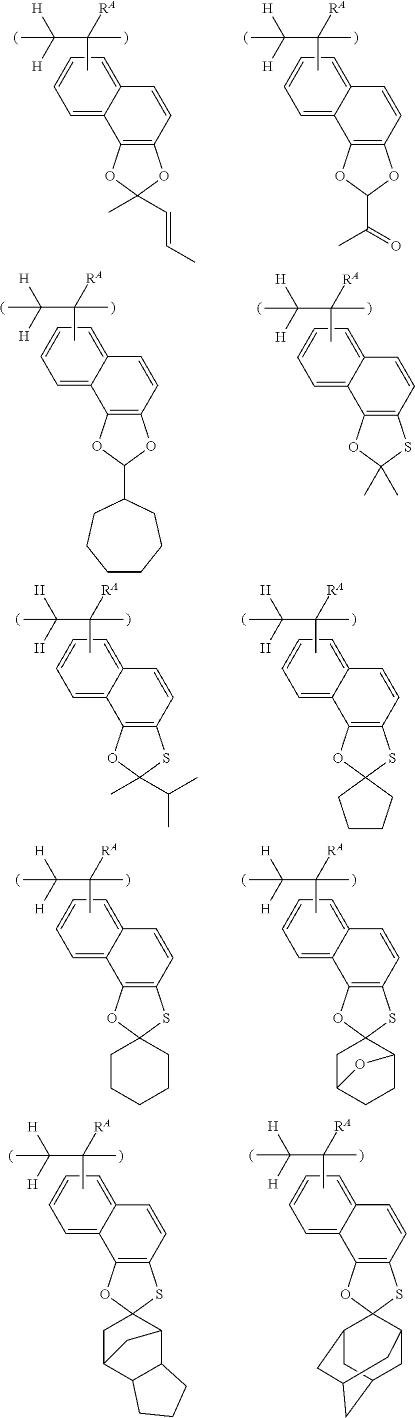

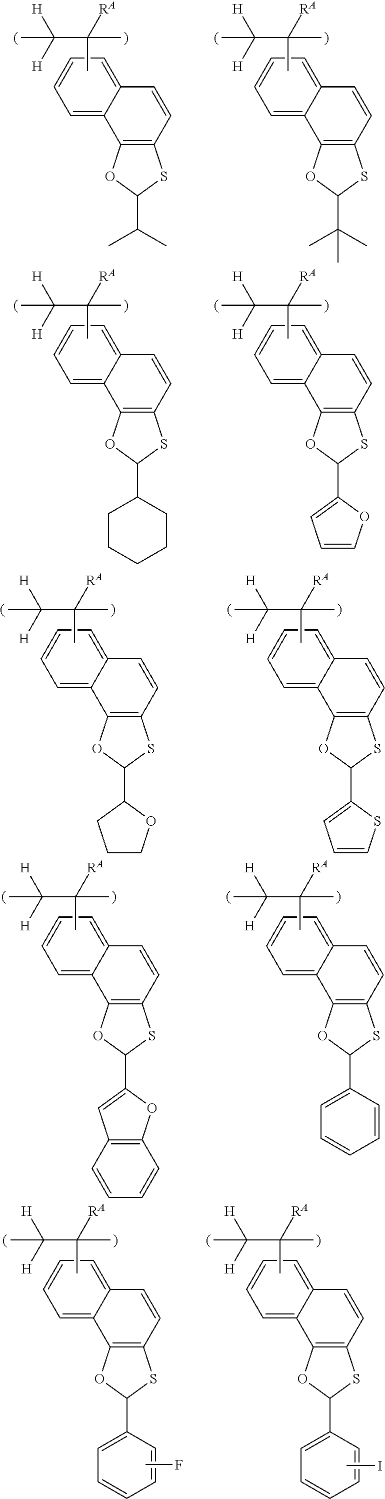

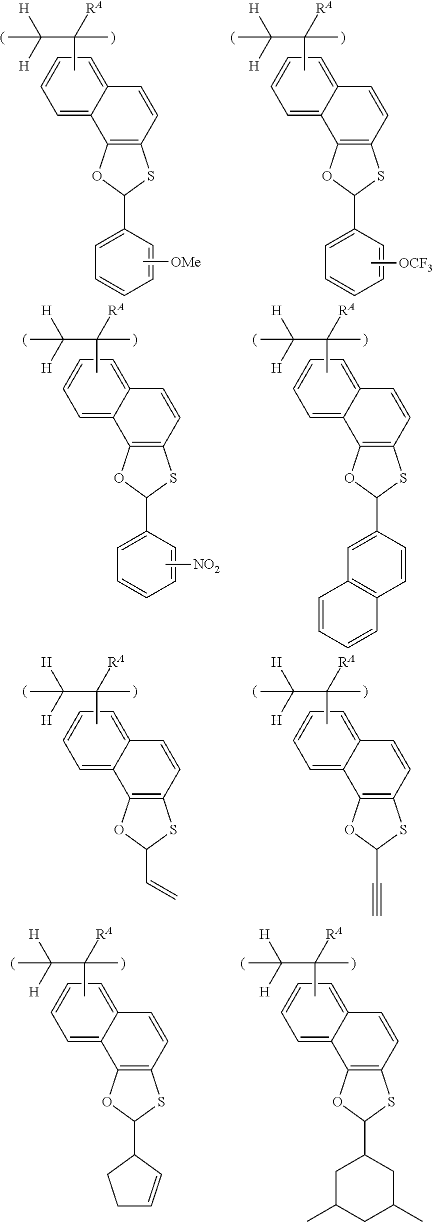

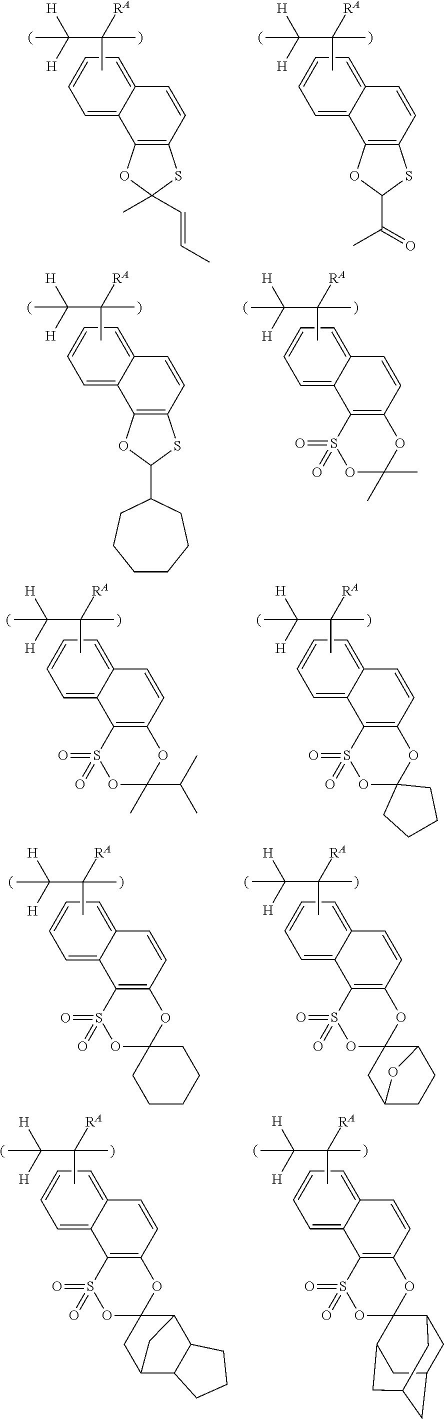

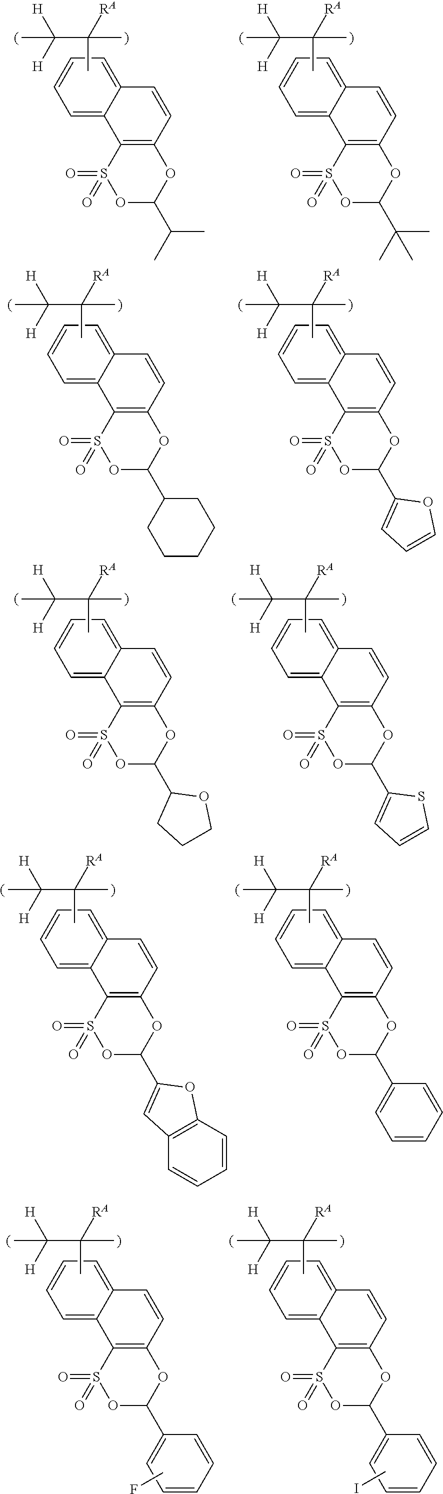

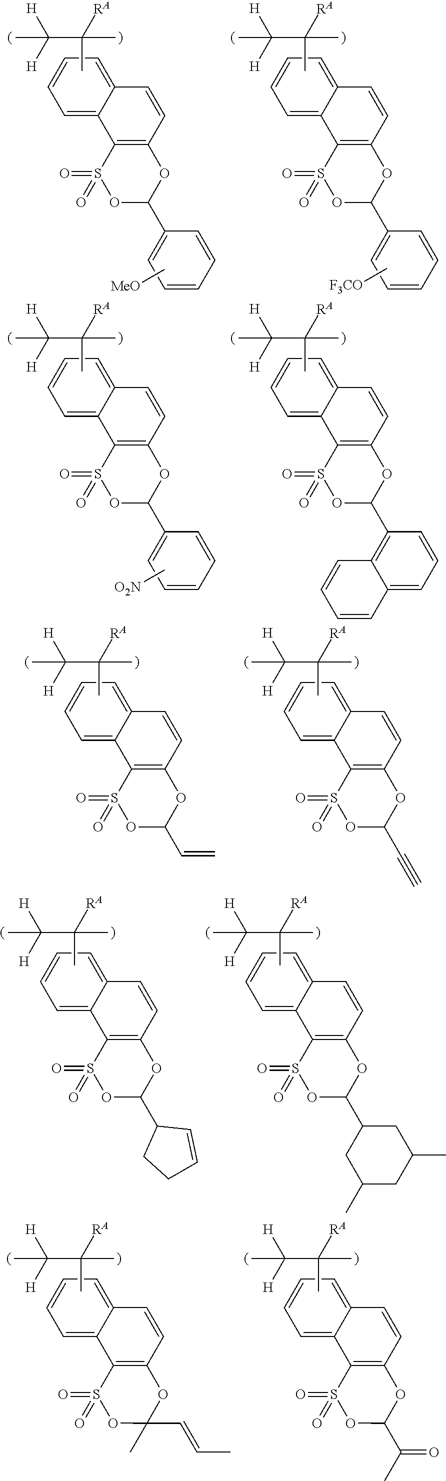

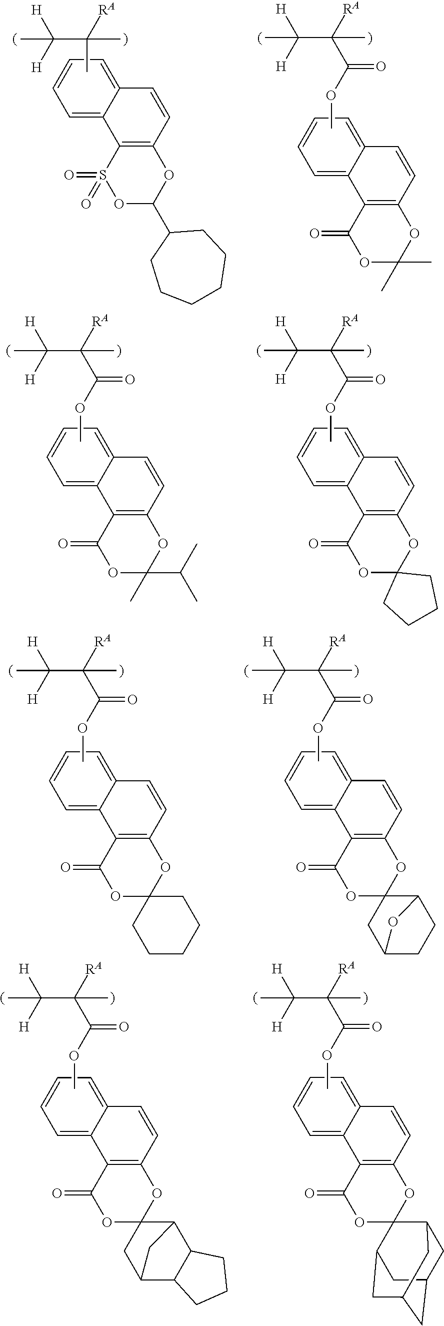

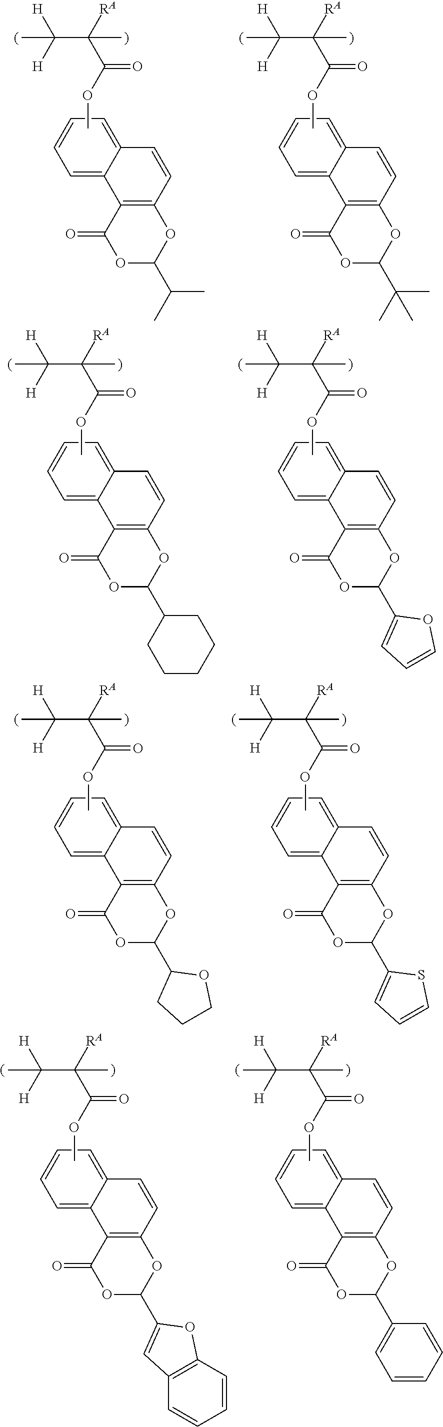

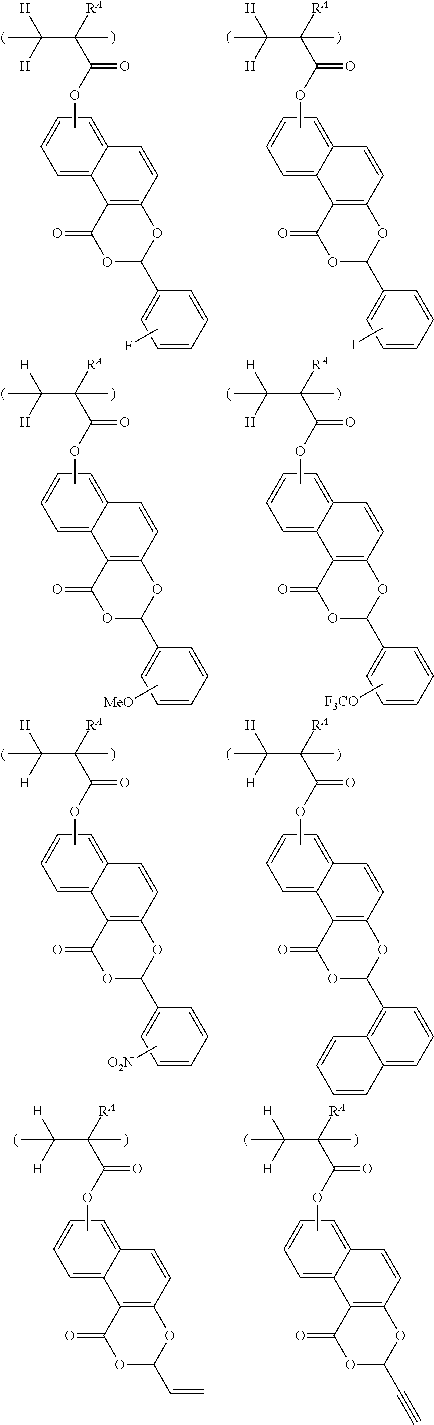

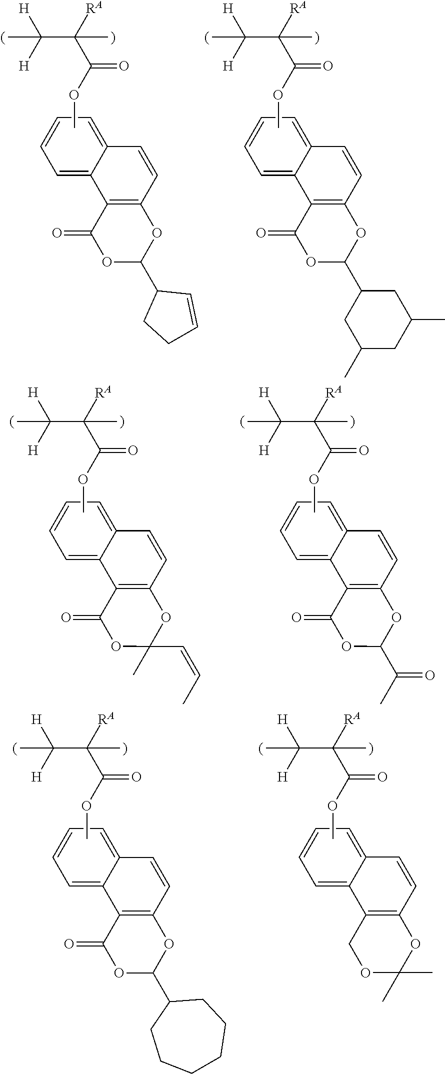

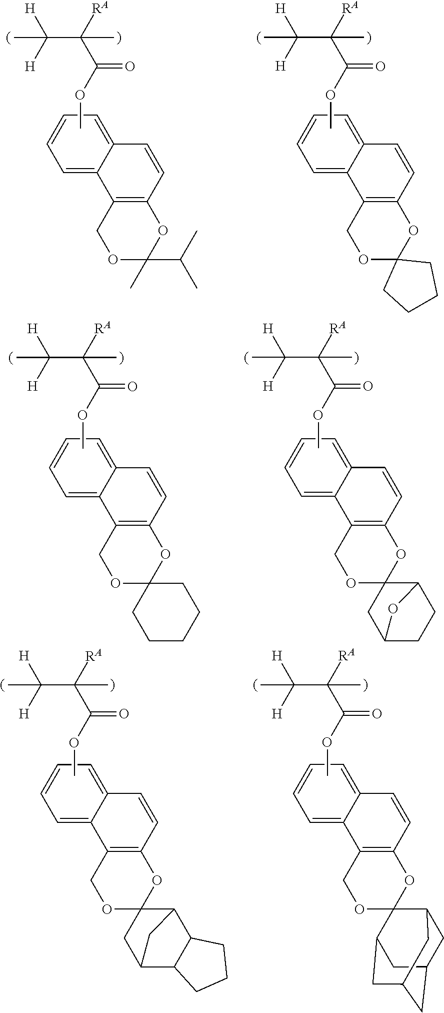

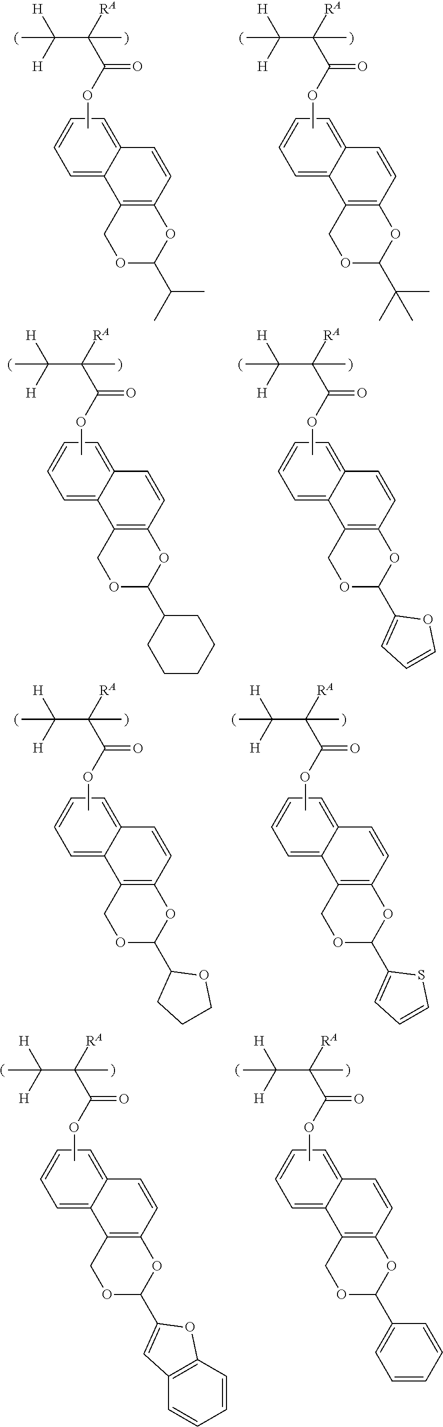

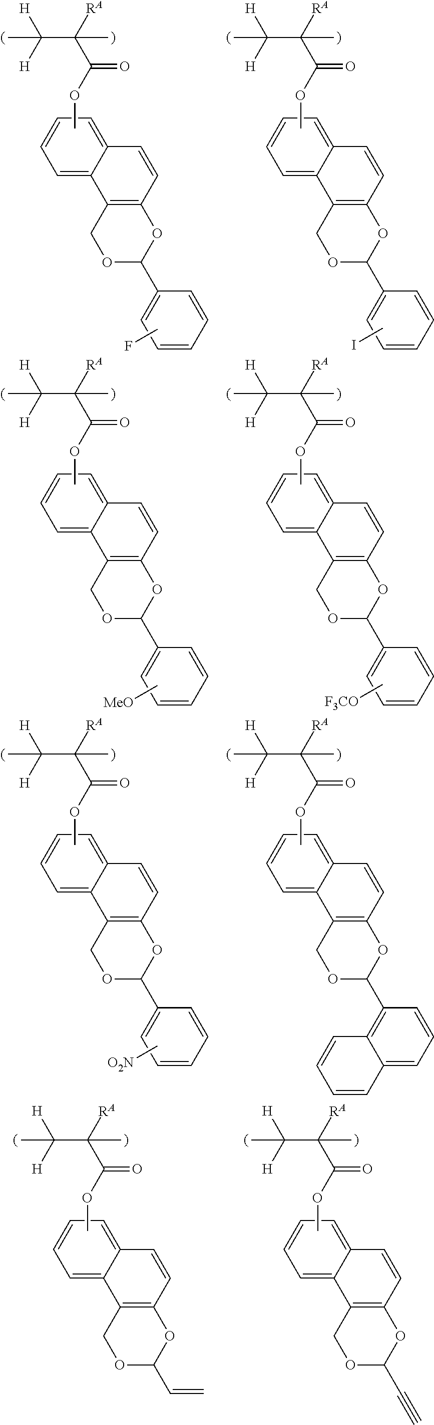

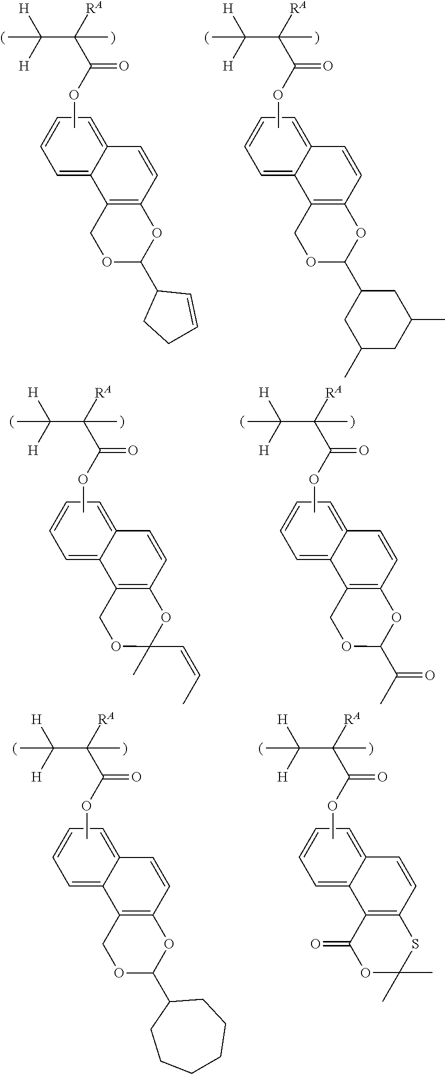

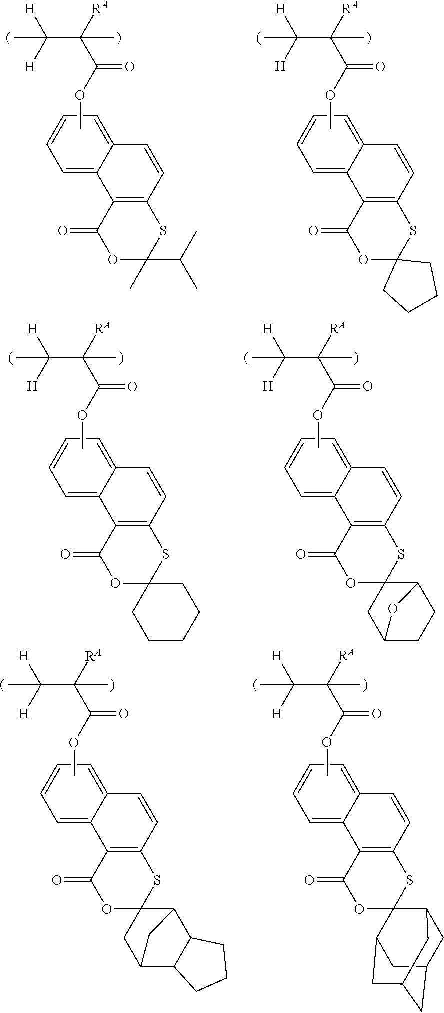

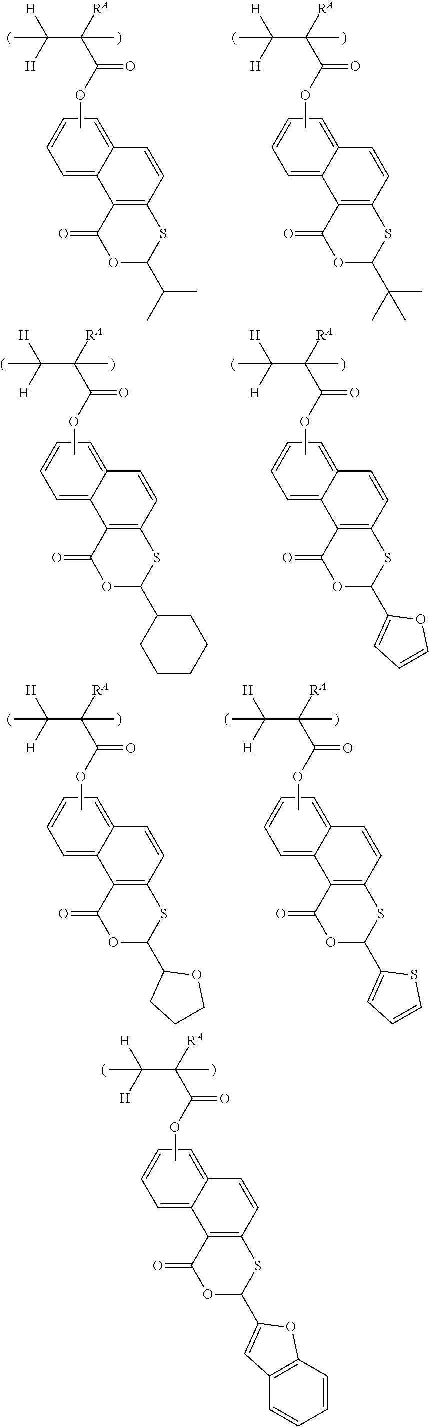

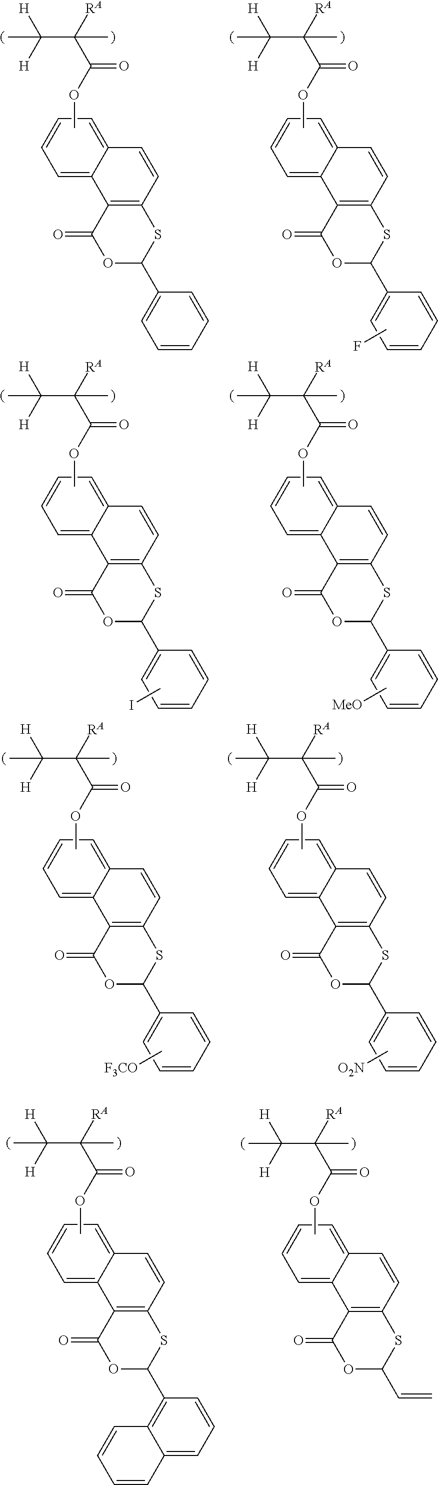

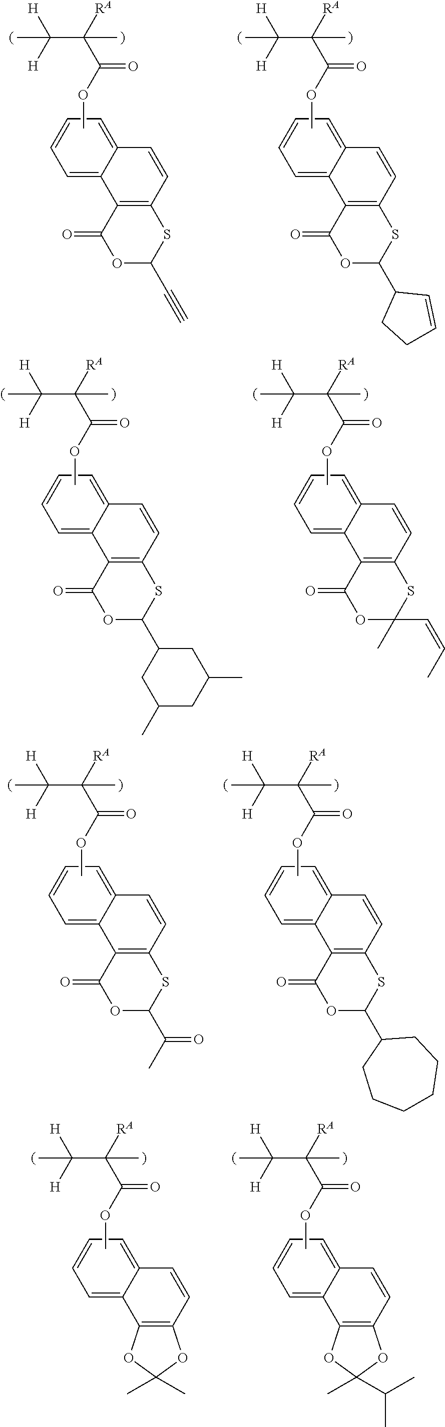

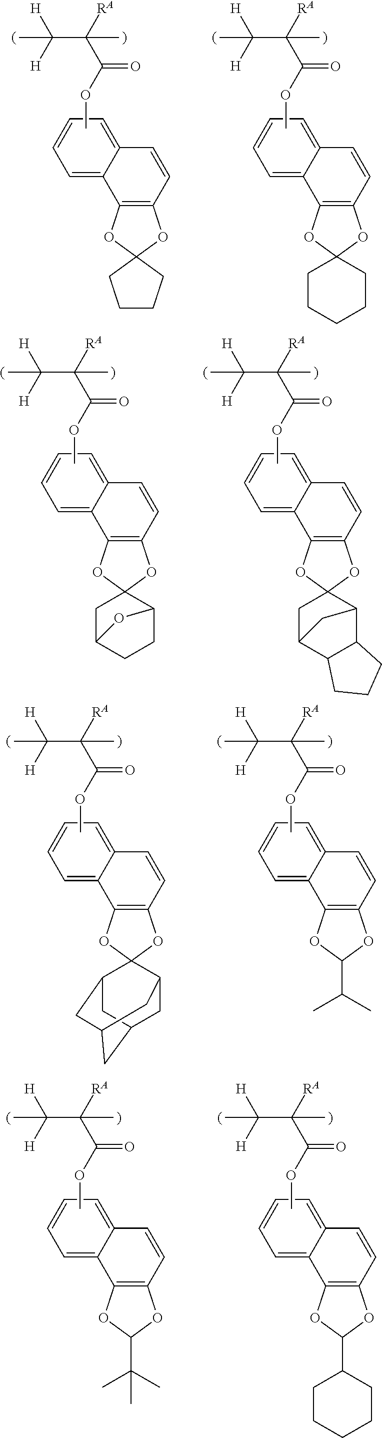

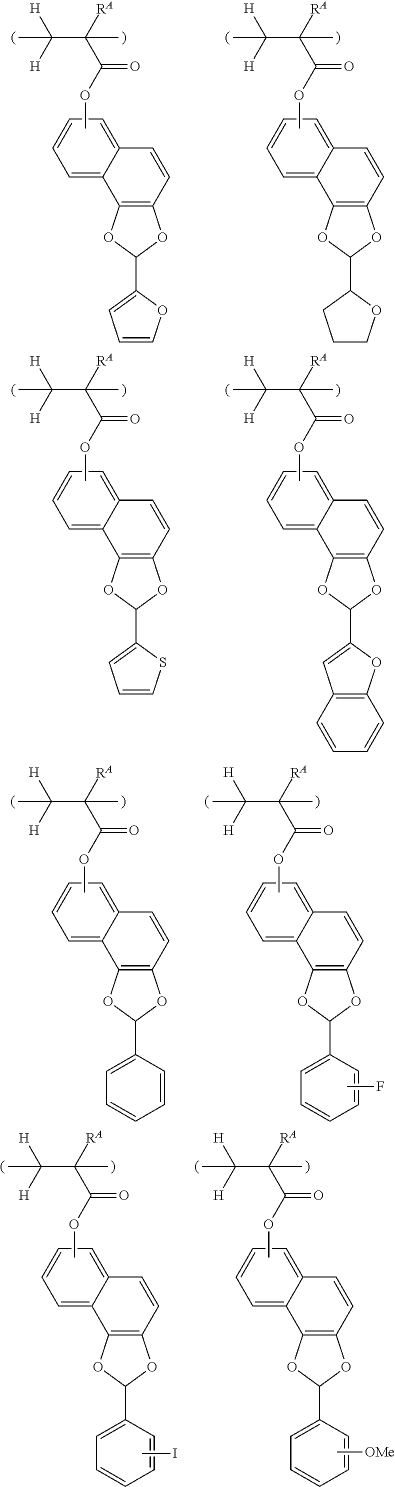

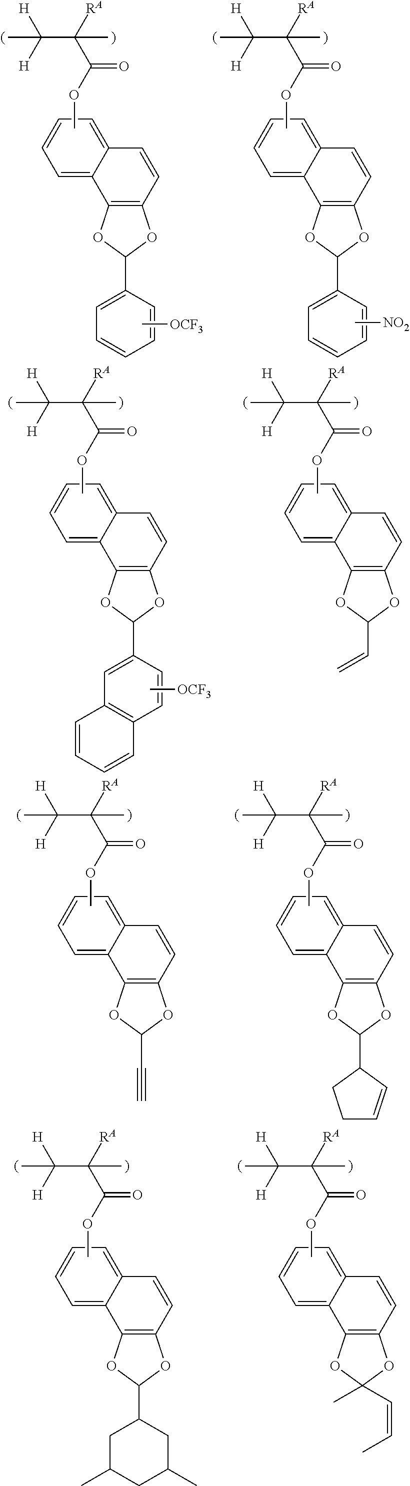

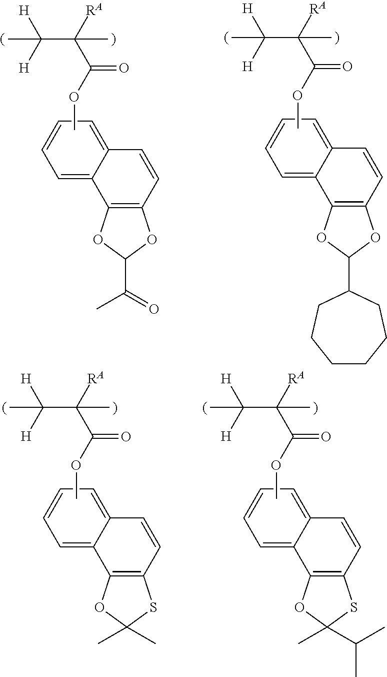

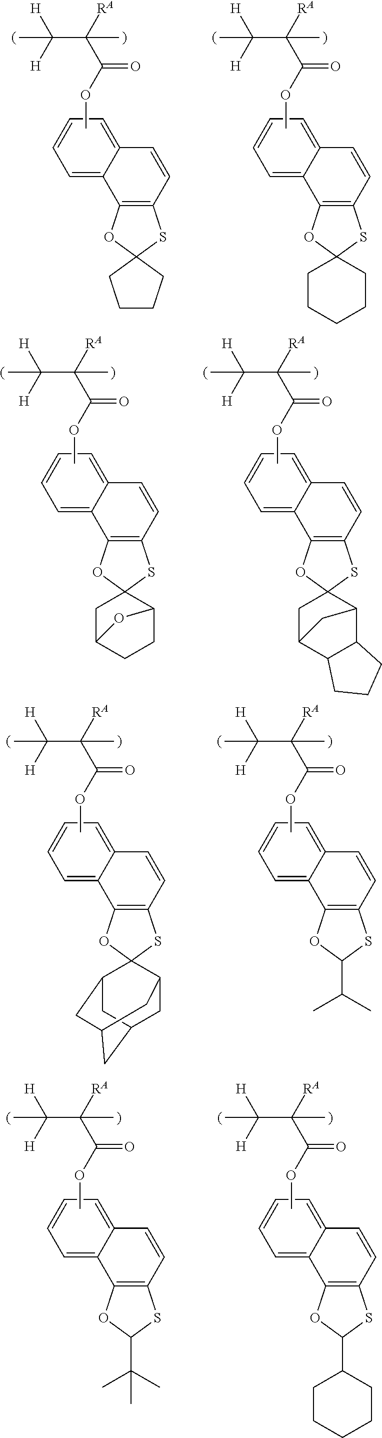

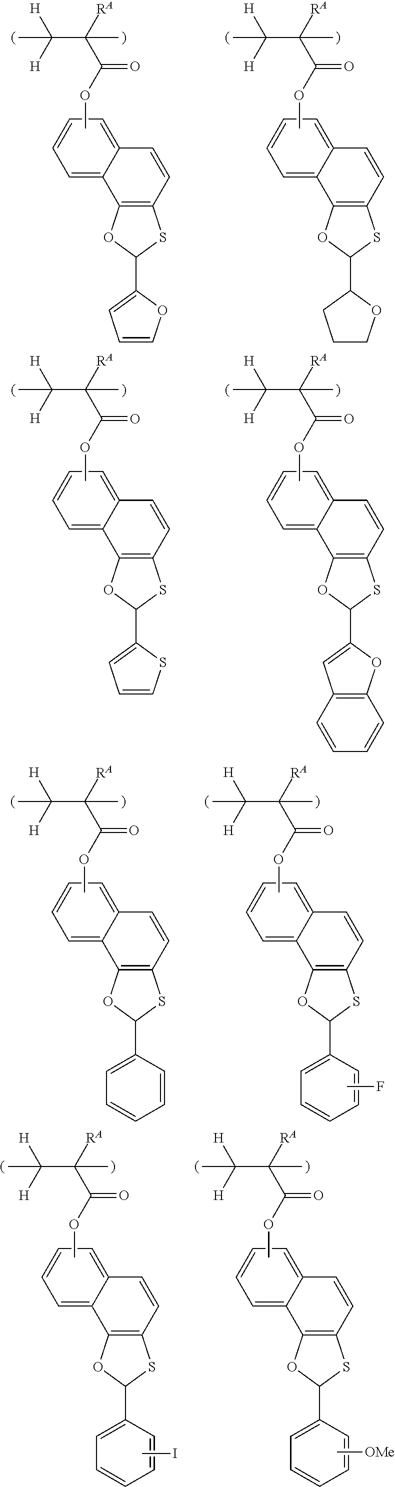

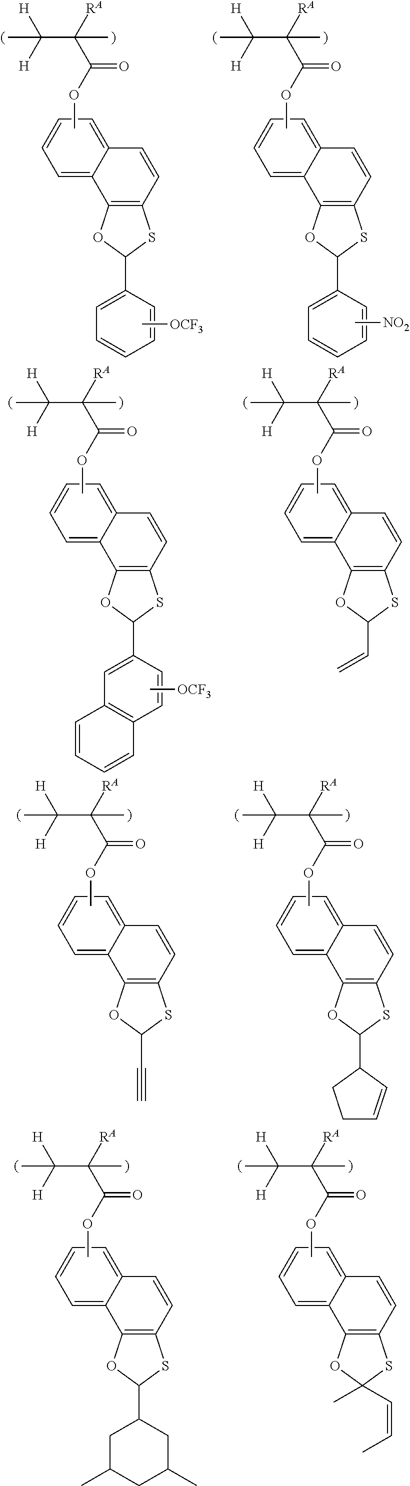

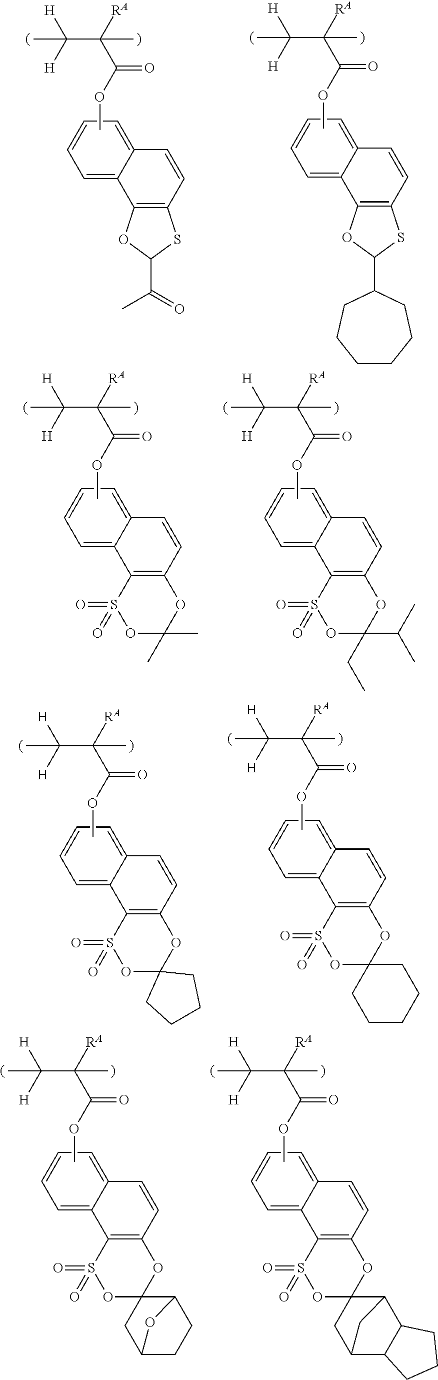

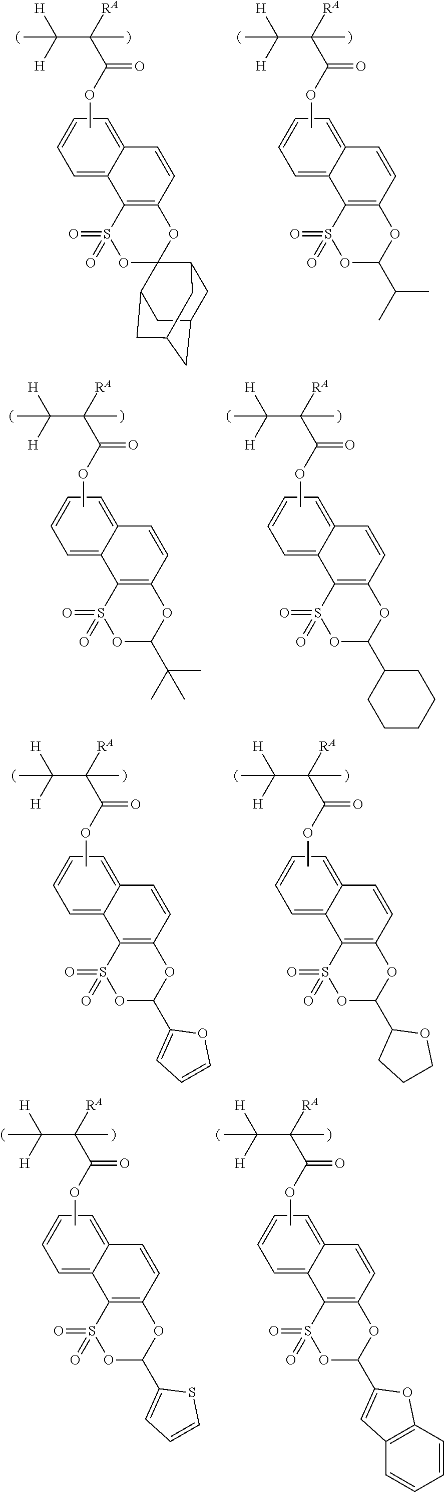

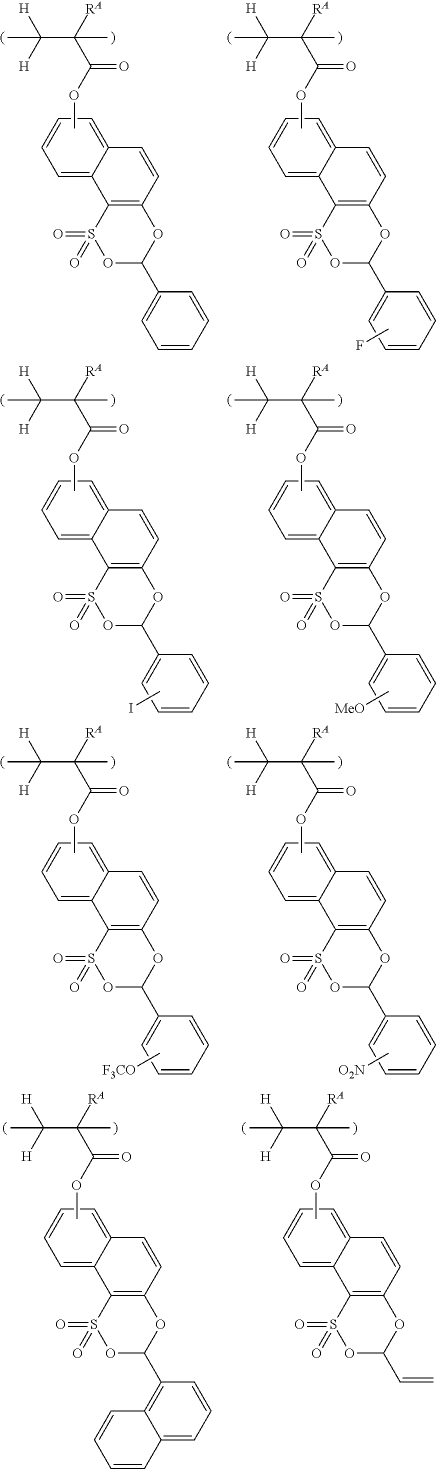

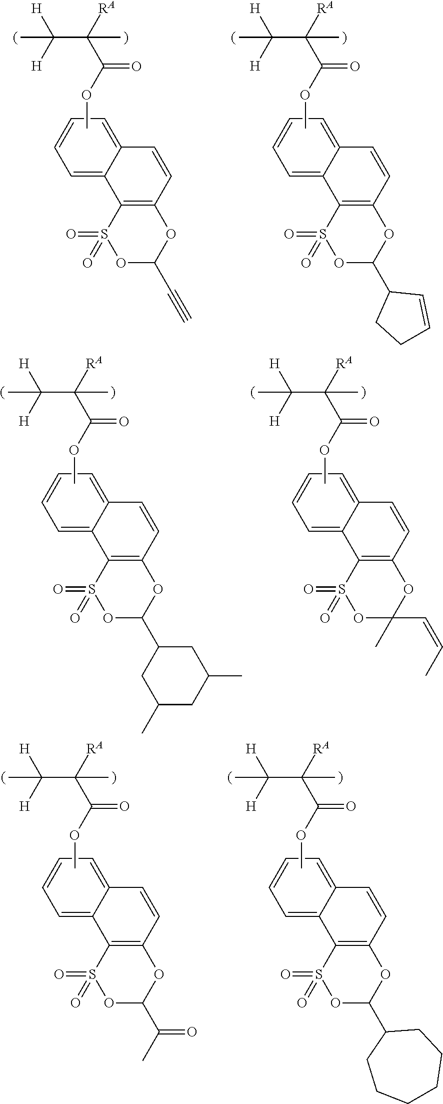

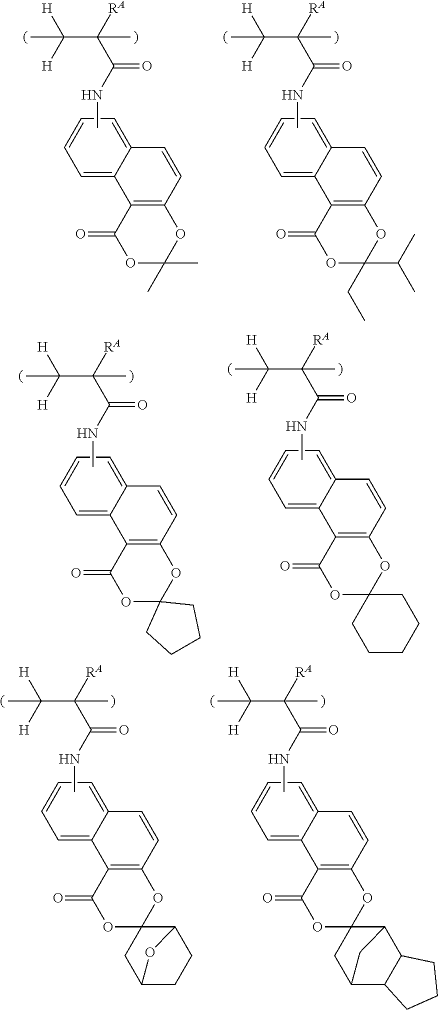

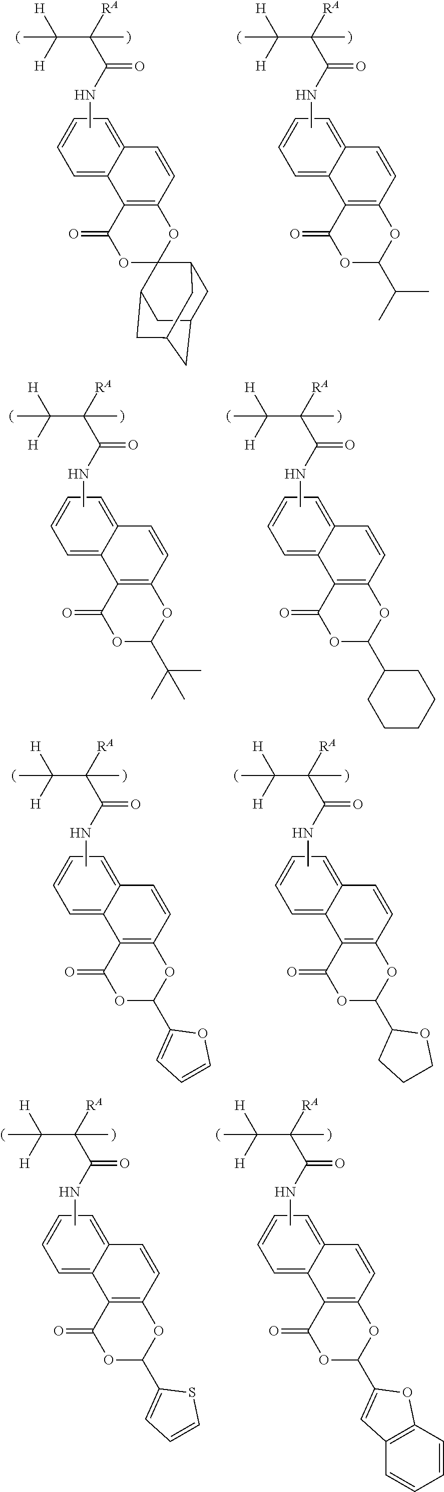

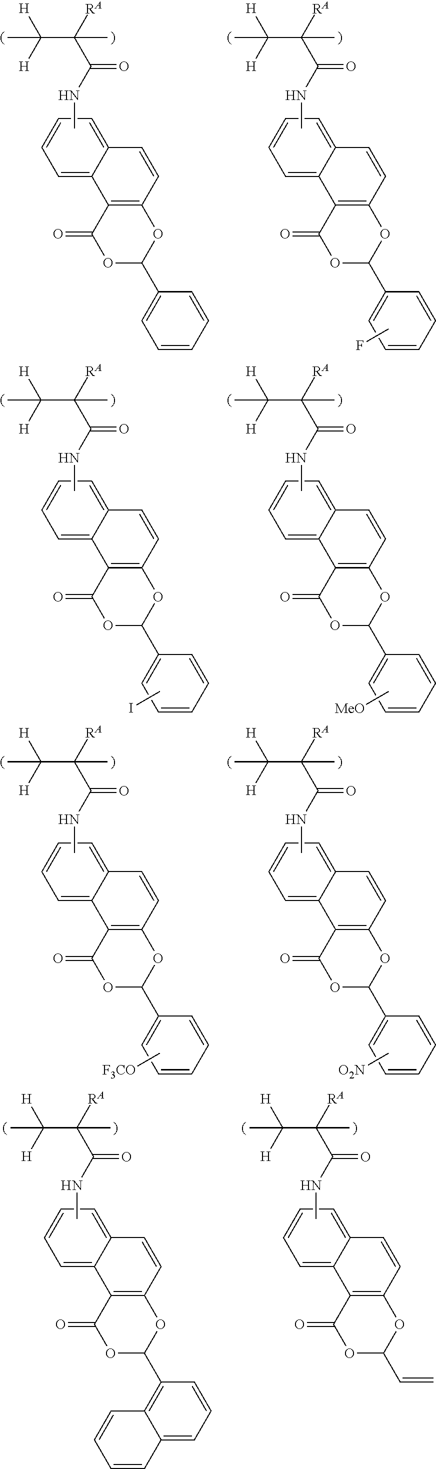

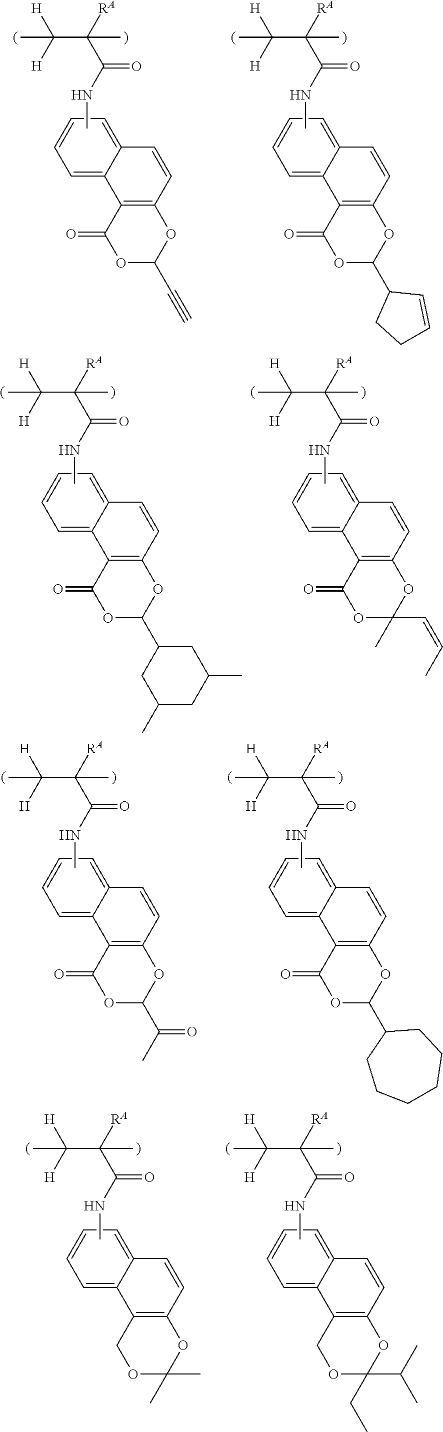

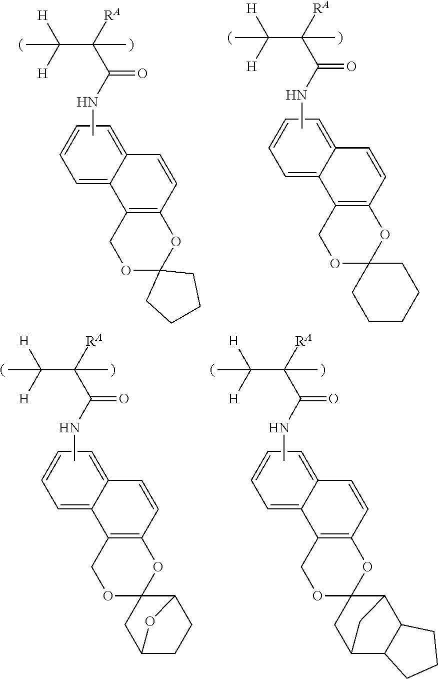

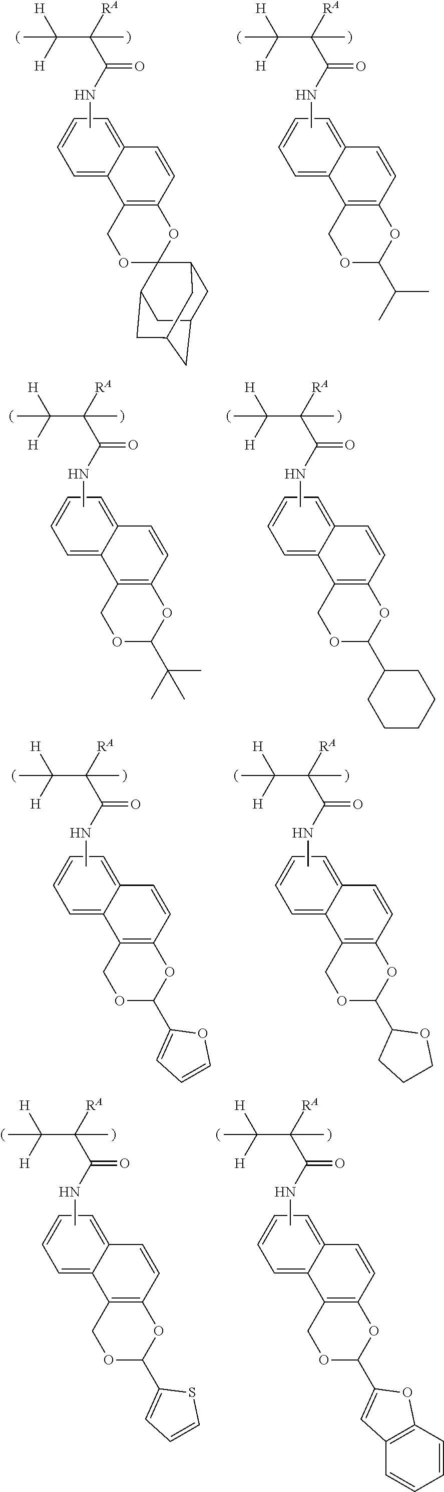

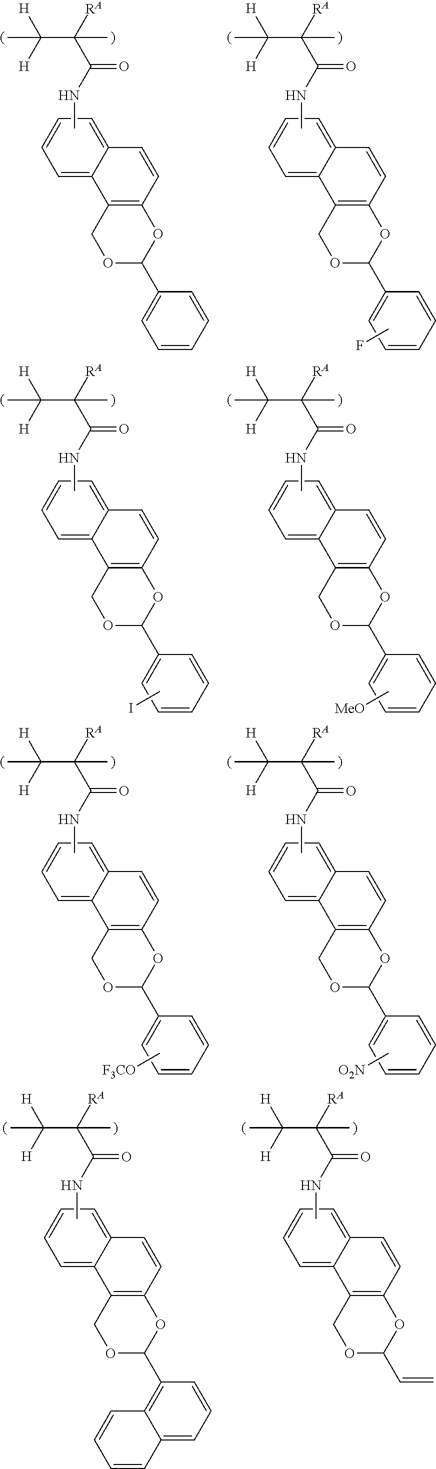

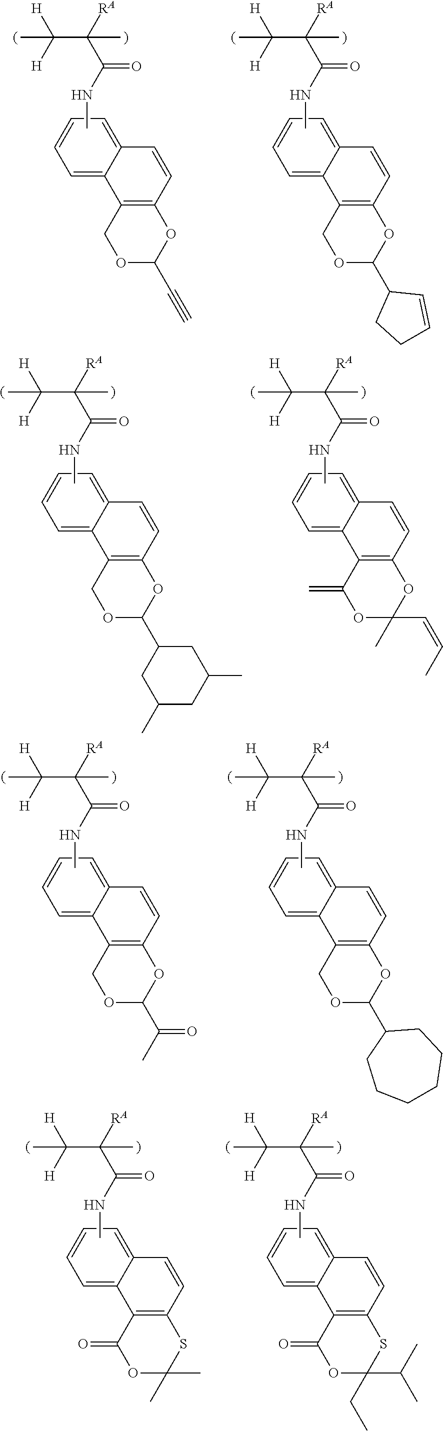

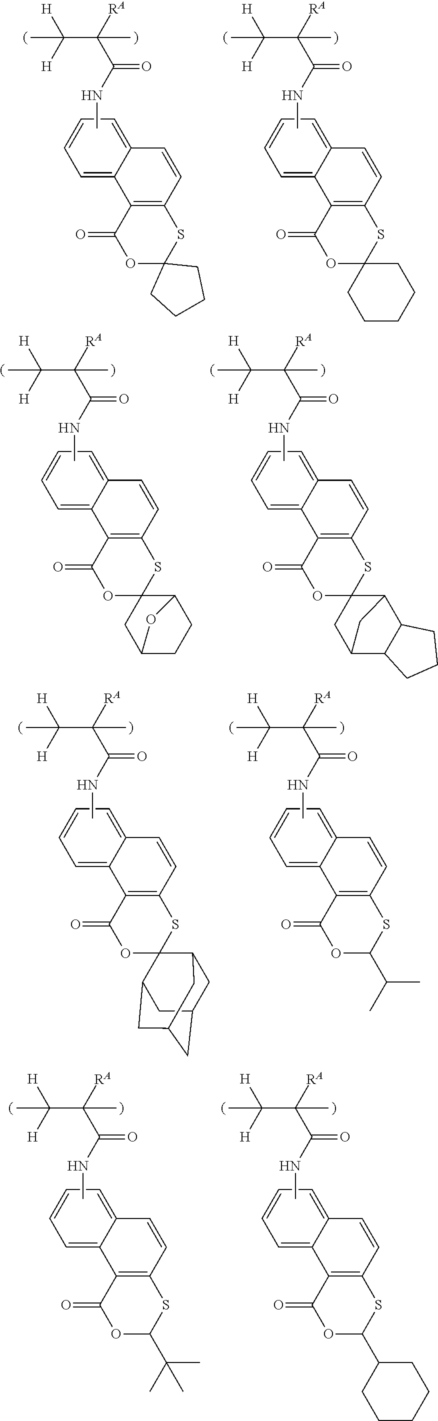

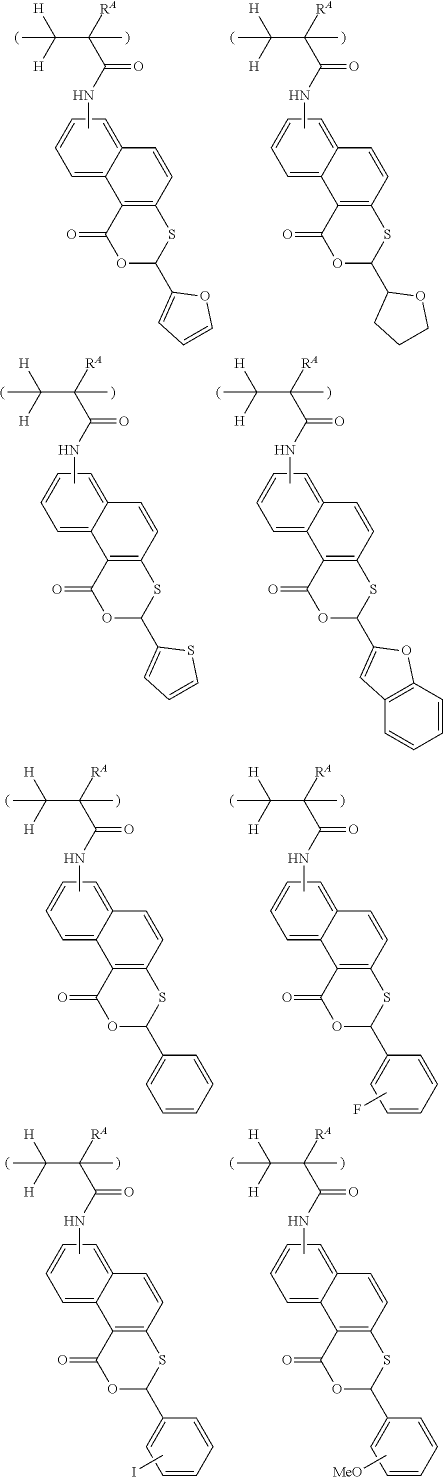

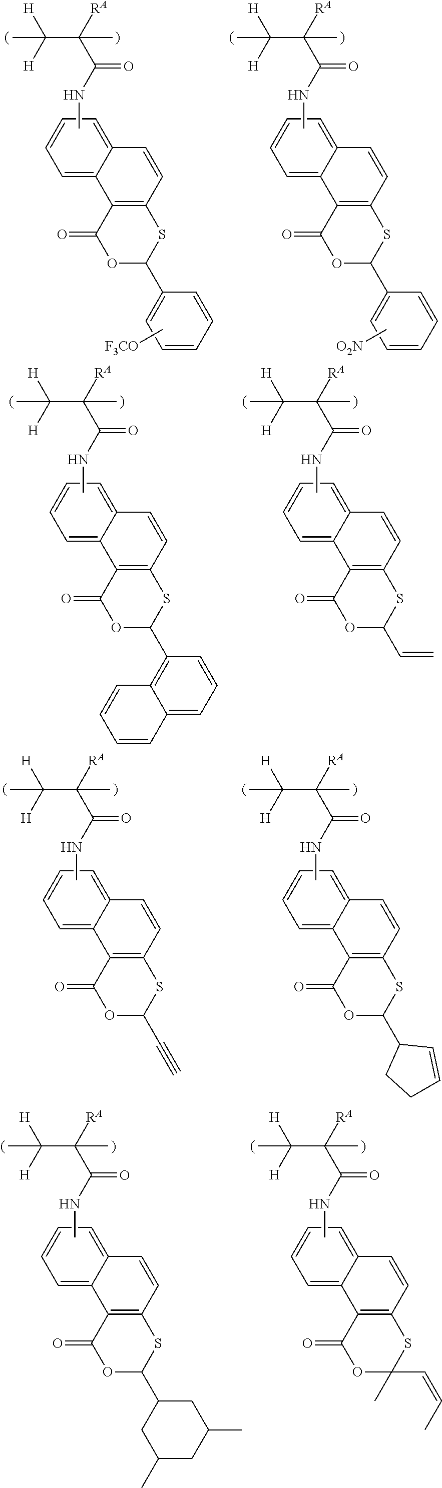

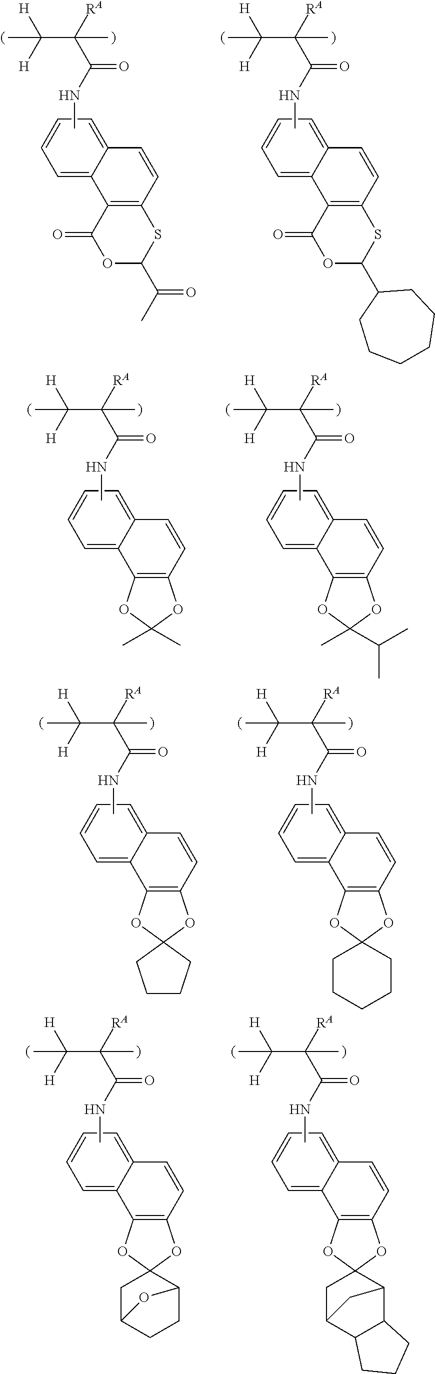

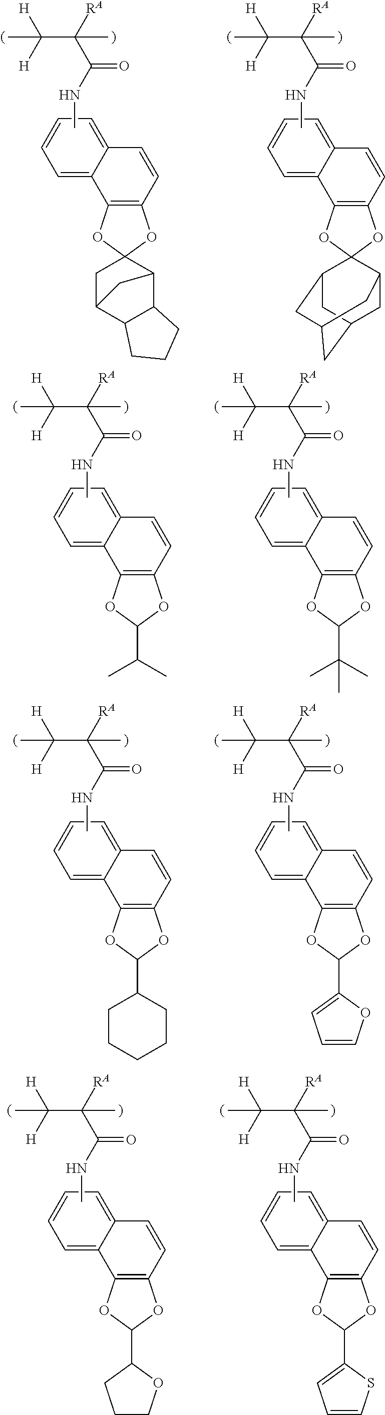

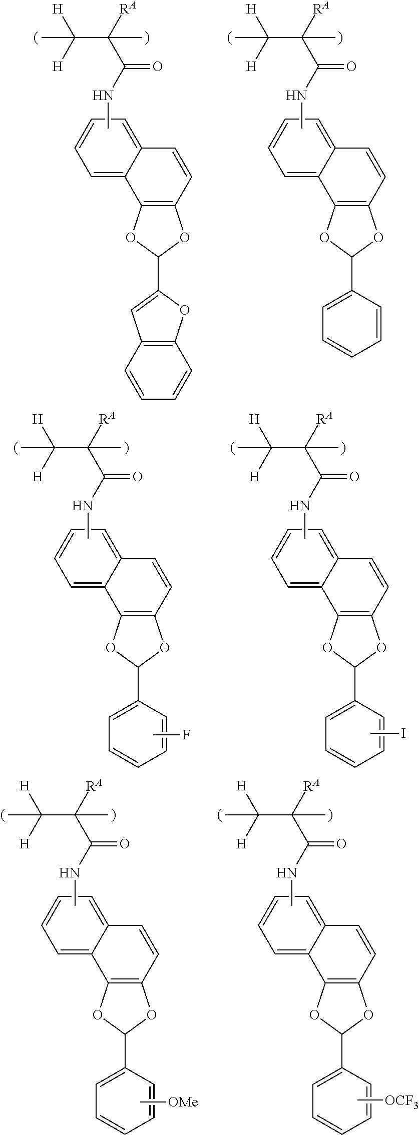

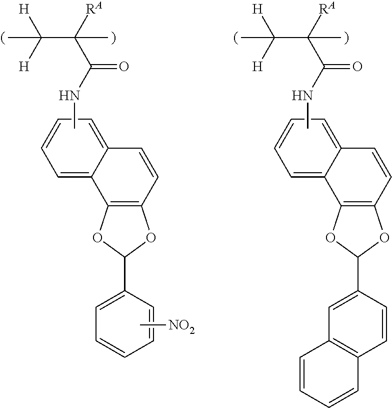

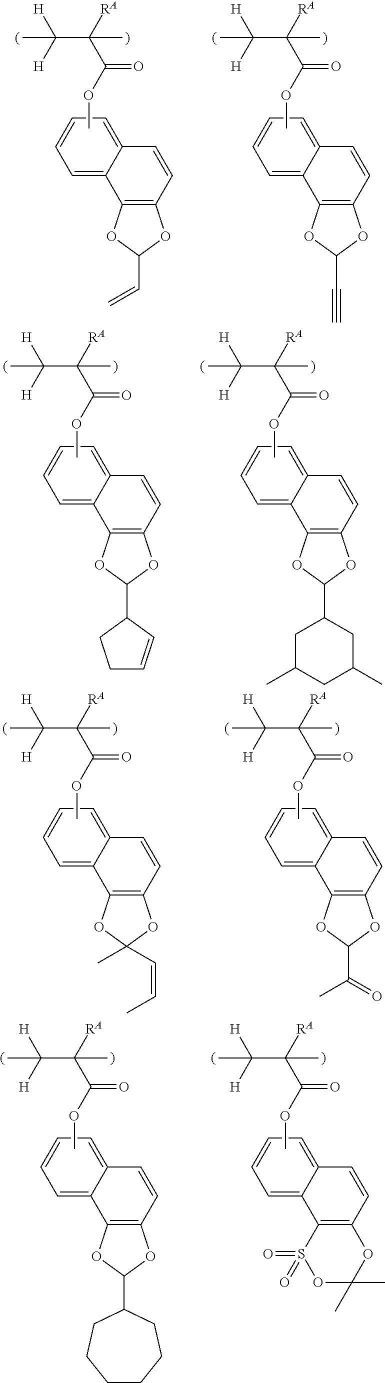

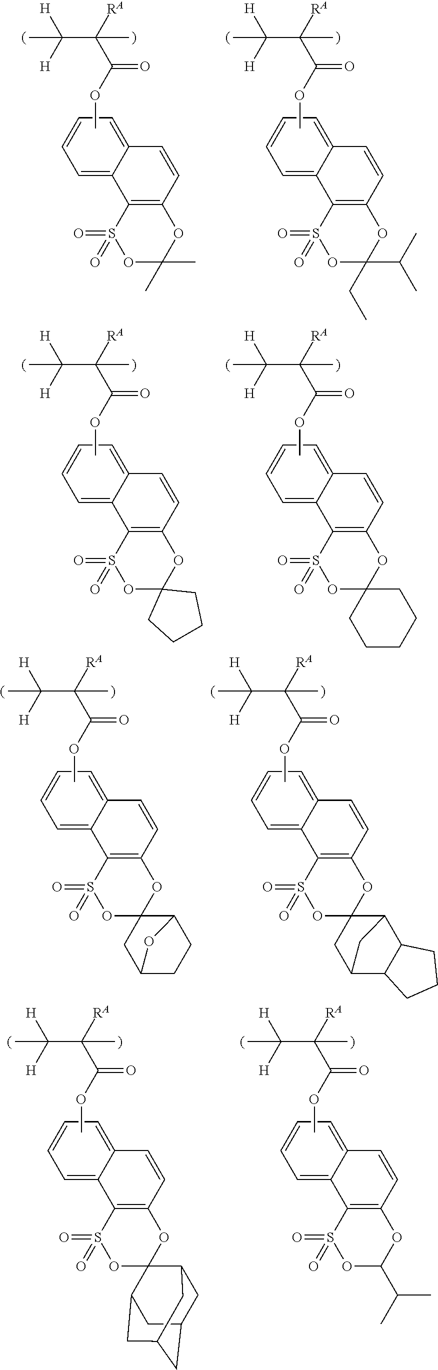

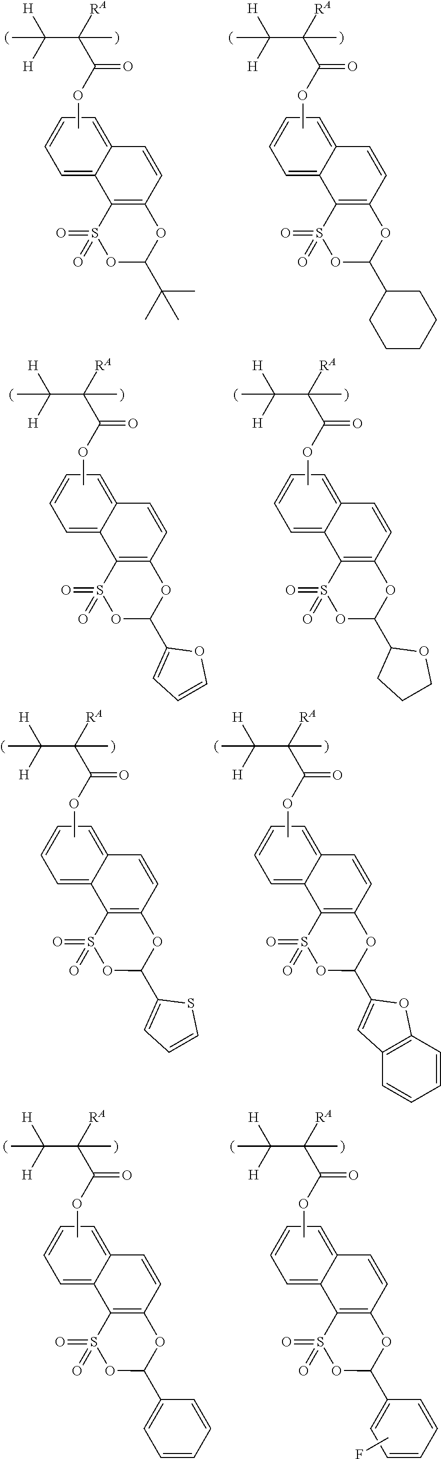

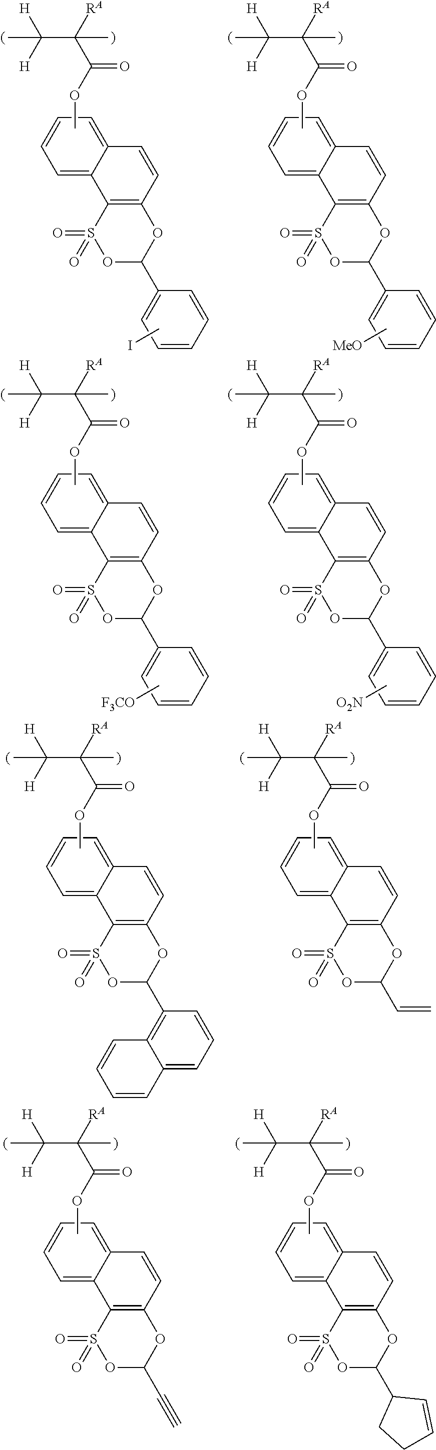

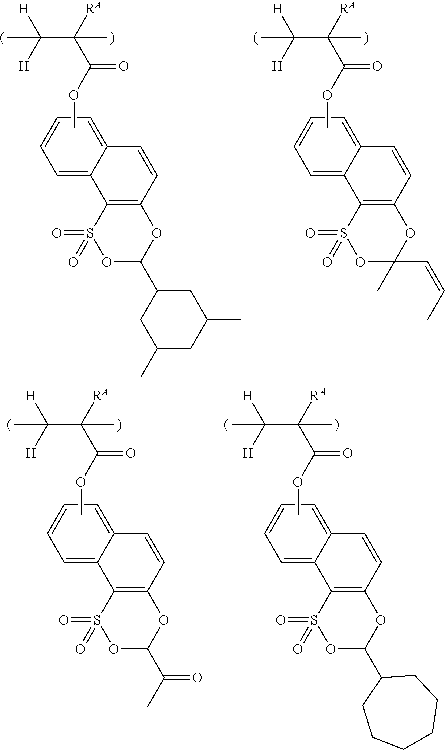

The base polymer preferably further contains a repeat unit having the formula (b1) (hereinafter also referred to as repeat unit b1) or a repeat unit having the formula (b2) (hereinafter also referred to as repeat unit b2).

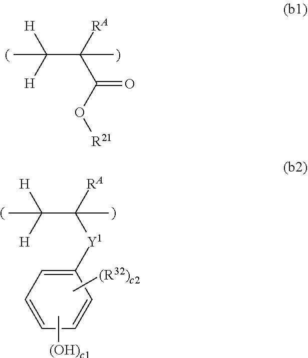

In the formulae (b1) and (b2), R^{A} s are each independently a hydrogen atom, a fluorine atom, a methyl group, or a trifluoromethyl group, Y¹ is a single bond or *—C(═O)—O—* designates a point of attachment to a carbon atom in the backbone. R³¹ is a hydrogen atom or a C₁-C₂₀ group containing at least one or more structures selected from a hydroxy group other than a phenolic hydroxy group, a cyano group, a carbonyl group, a carboxy group, an ether bond, an ester bond, a sulfonate ester bond, a carbonate bond, a lactone ring, a sultone ring, and a carboxylic anhydride (—C(═O)—O—C(═O)—), R³² is a halogen atom, a hydroxy group, a carboxy group, a nitro group, a cyano group, a C₁-C₂₀ hydrocarbyl group which may contain a heteroatom, a C₁-C₂₀ hydrocarbyloxy group which may contain a heteroatom, a C₂-C₂₀ hydrocarbylcarbonyl group which may contain a heteroatom, a C₂-C₂₀ hydrocarbylcarbonyloxy group which may contain a heteroatom, or a C₂-C₂₀ hydrocarbyloxycarbonyl group which may contain a heteroatom, c1 is 1, 2, 3, or 4, c2 is 0, 1, 2, 3, or 4, provided that 1≤c1+c2≤5.

Specific examples of the repeat unit b1 include those shown below, but are not limited thereto. In the following formulae, R¹ is as defined above.

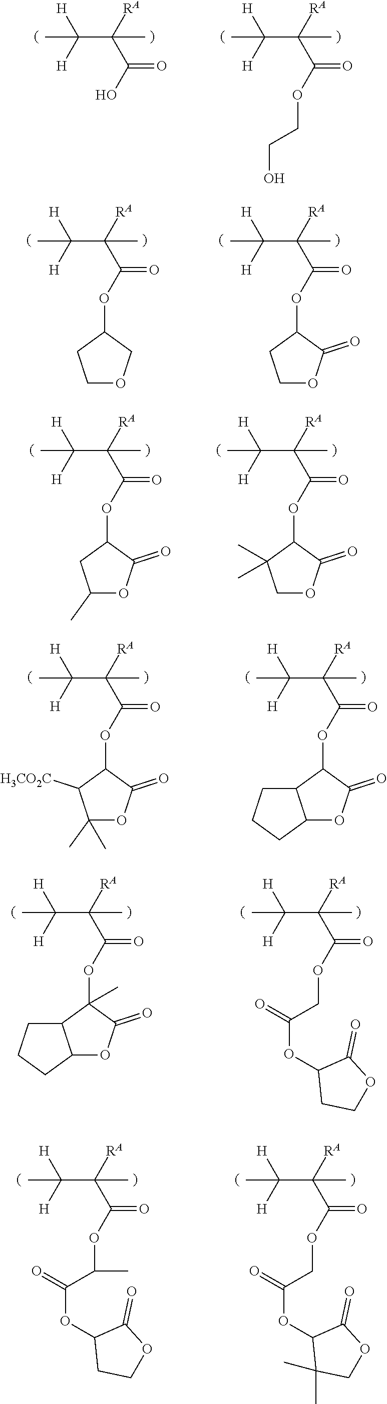

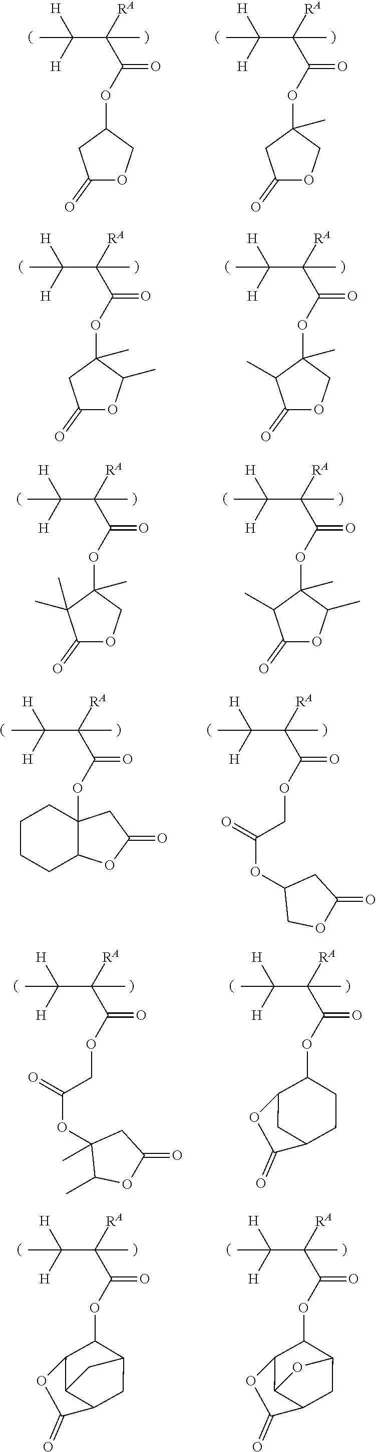

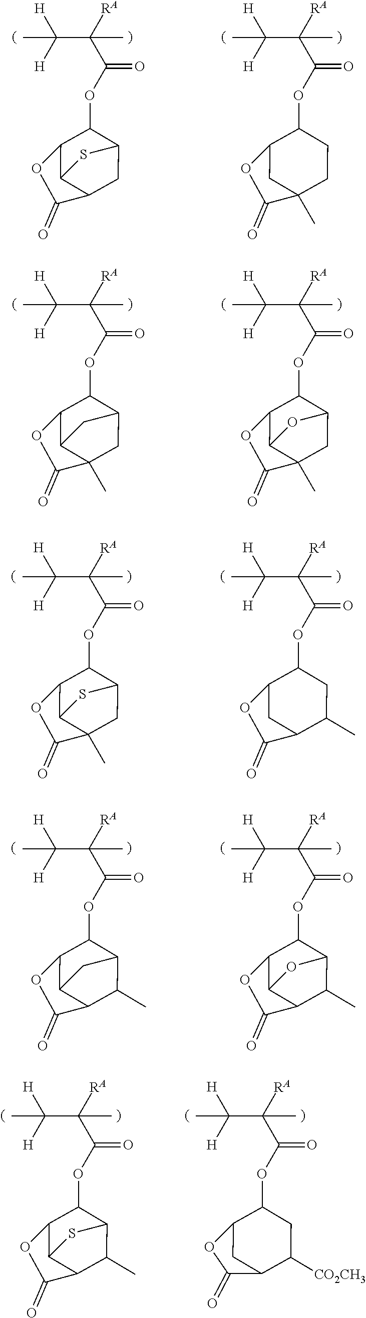

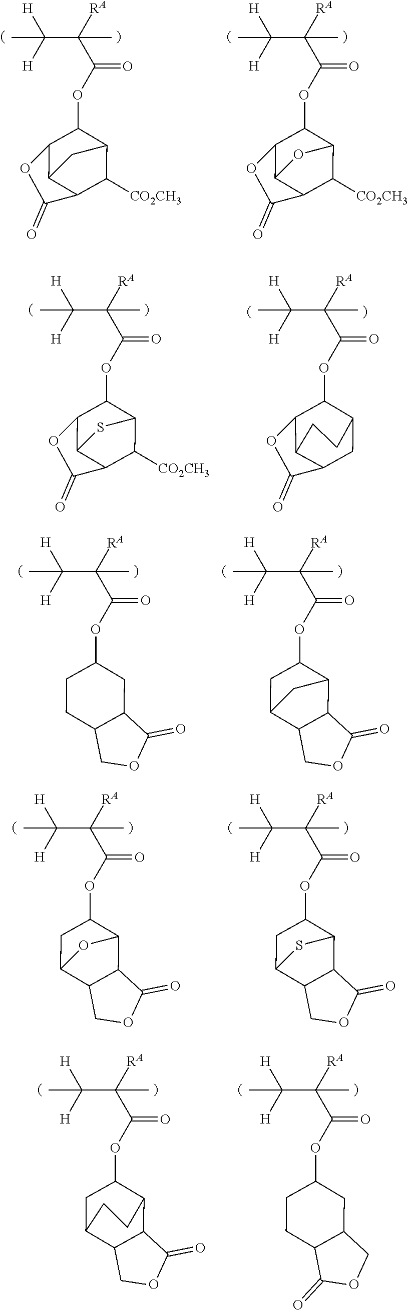

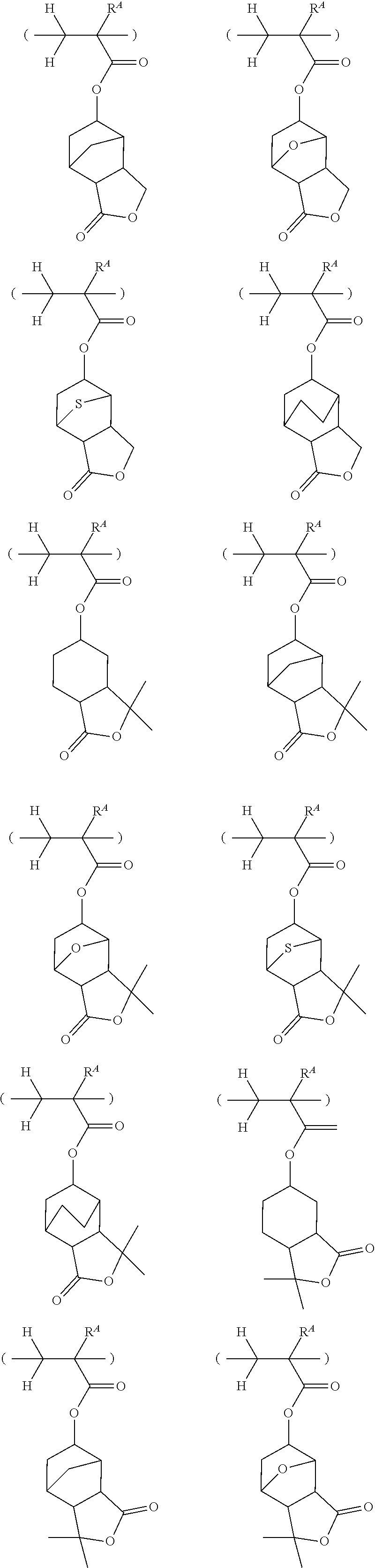

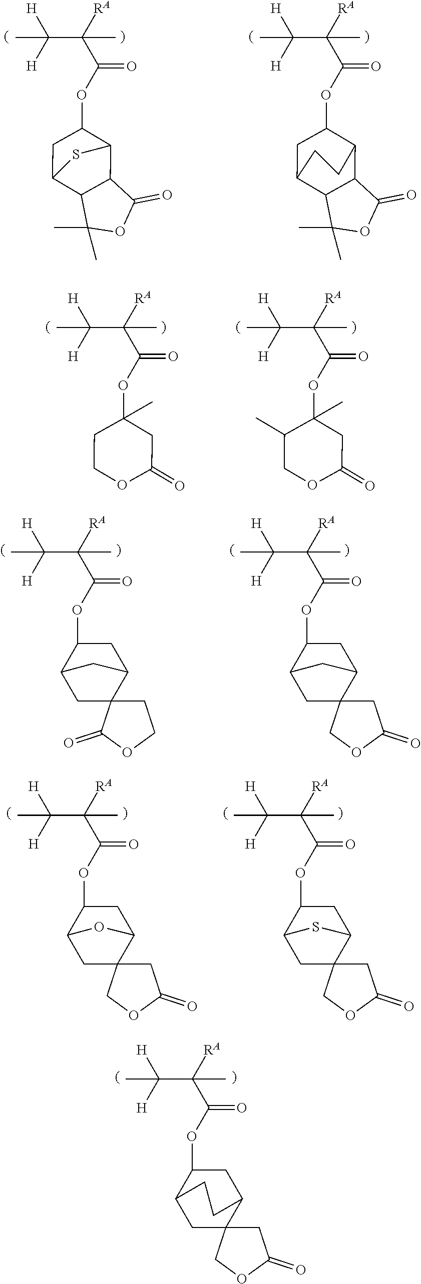

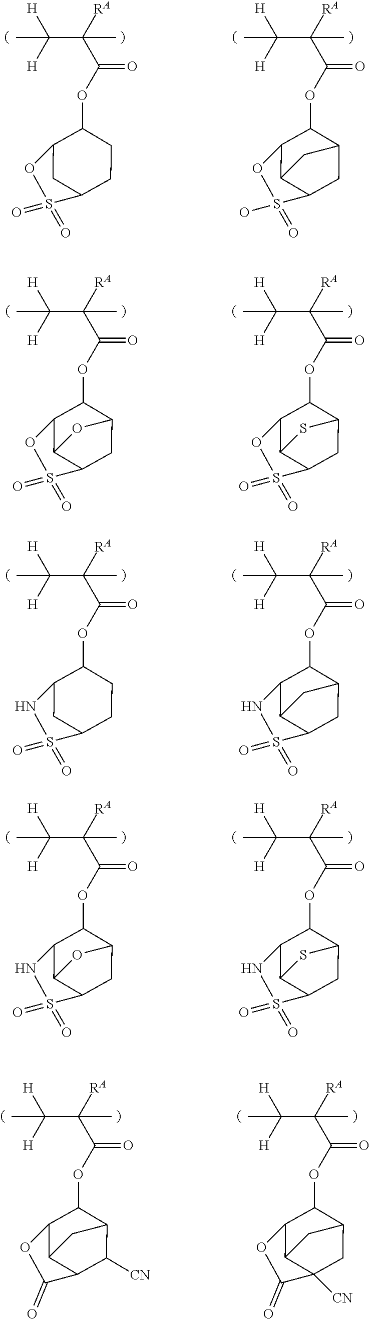

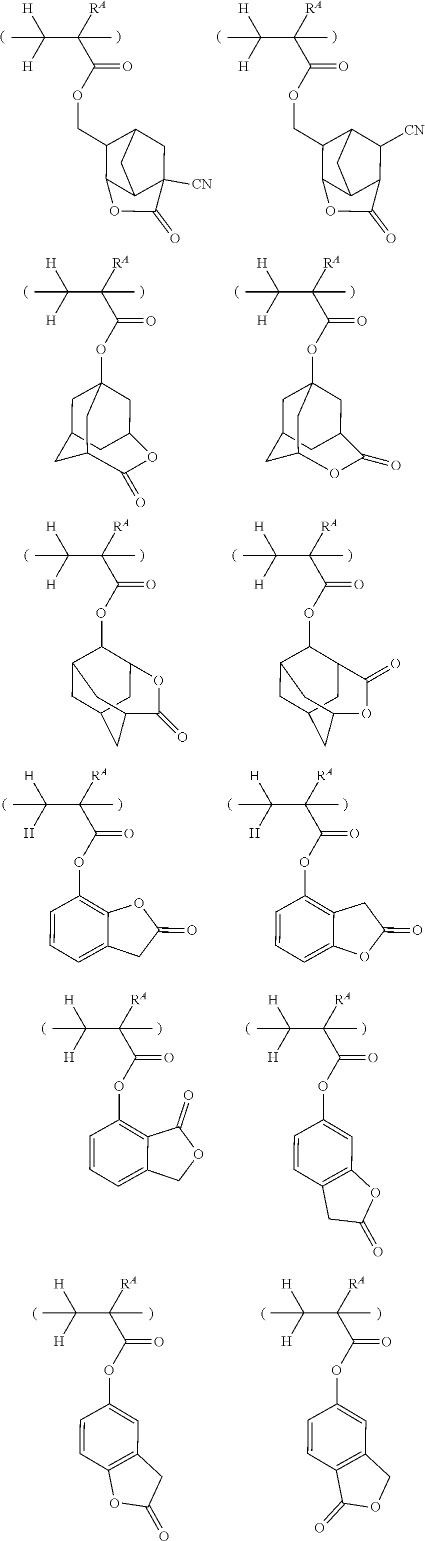

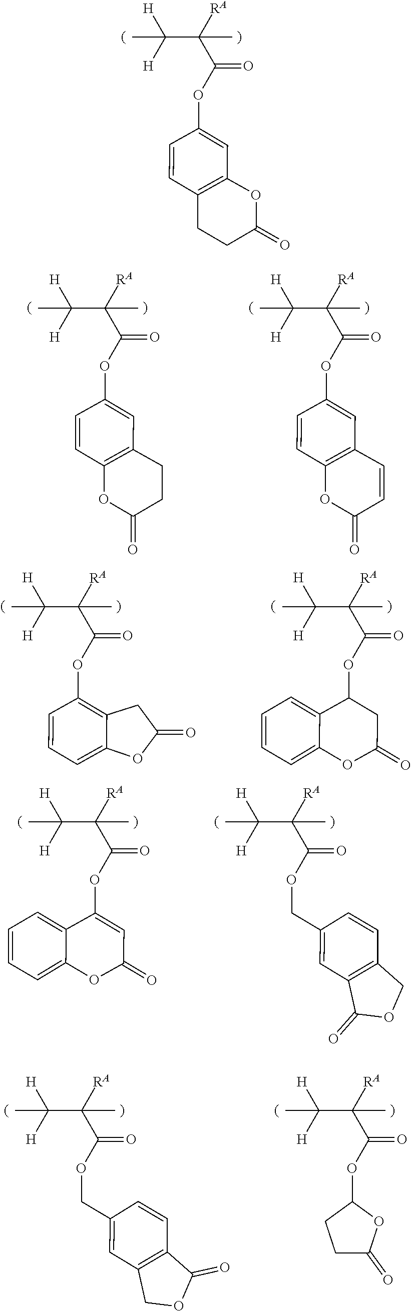

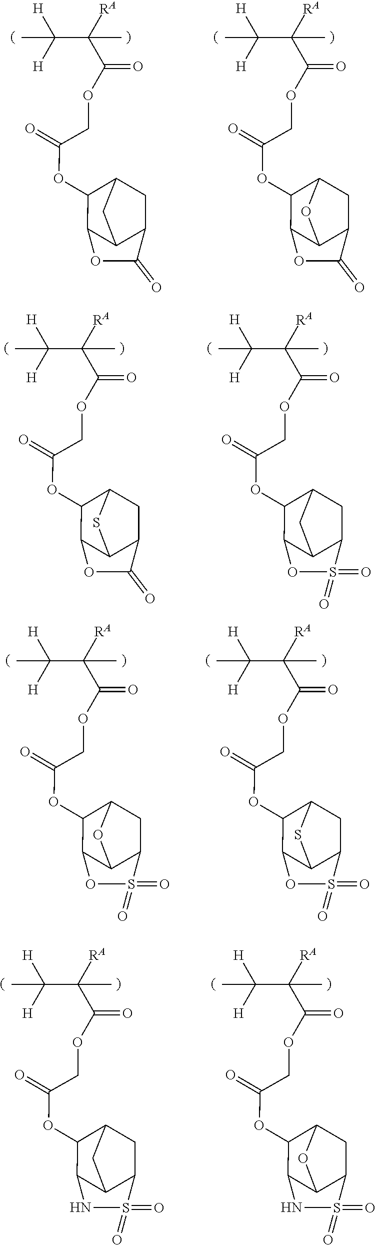

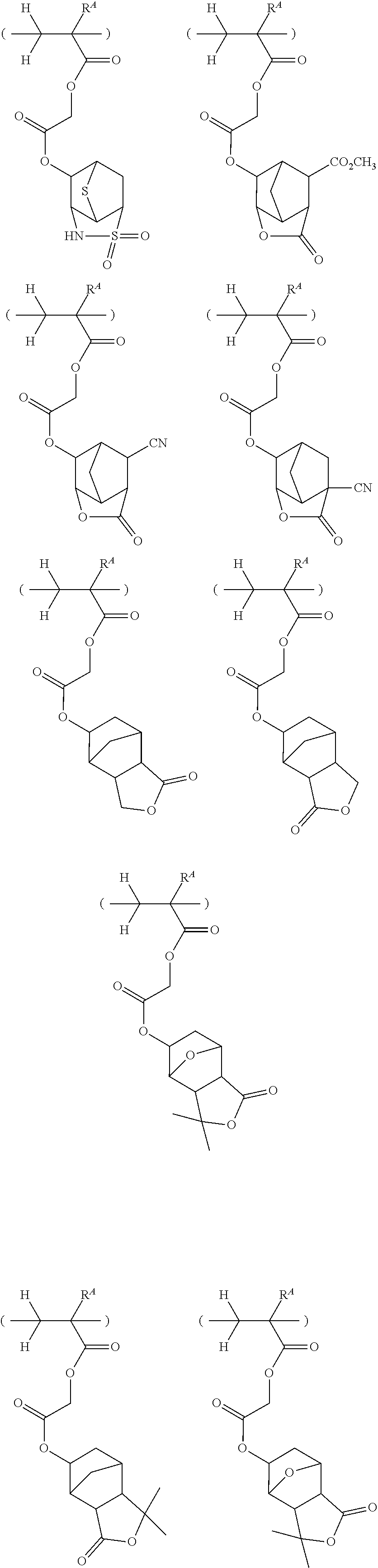

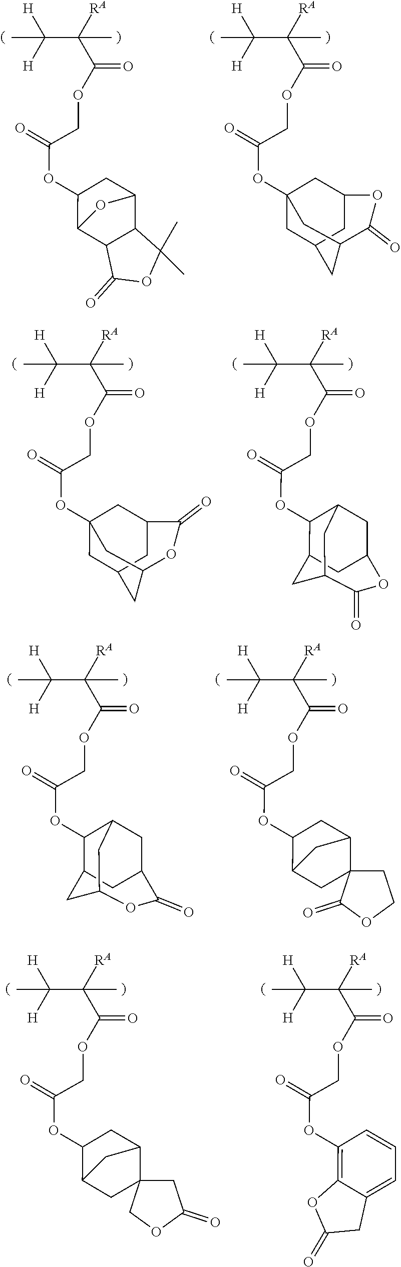

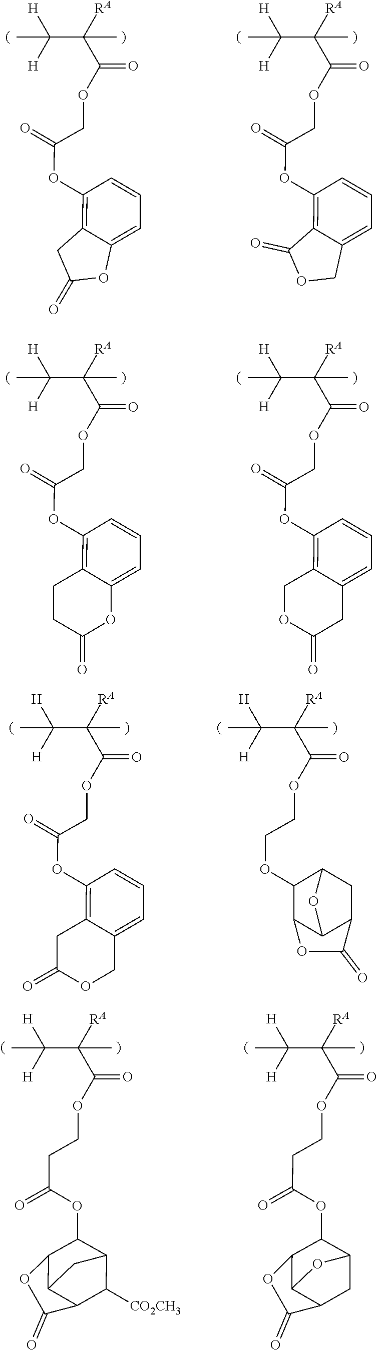

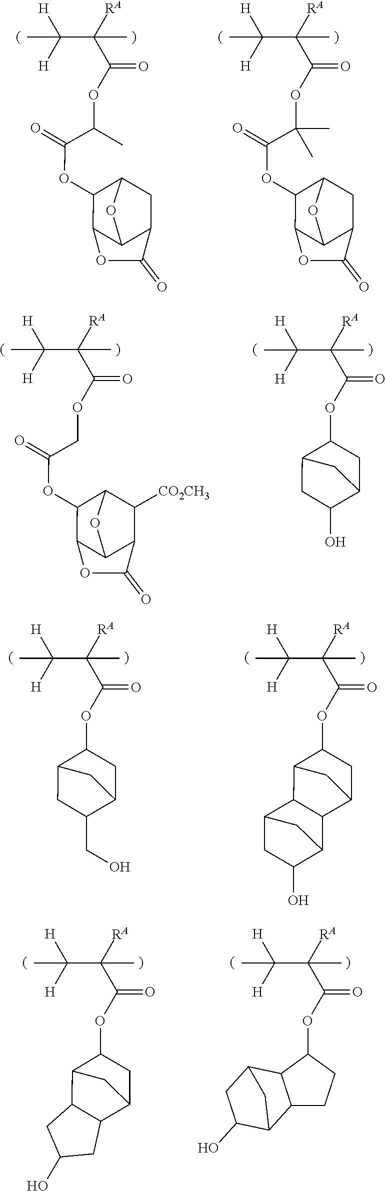

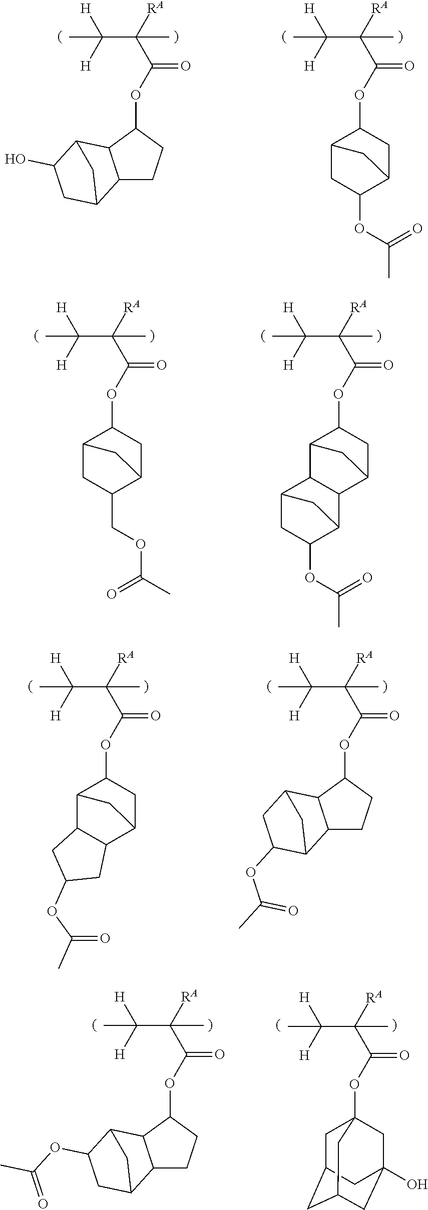

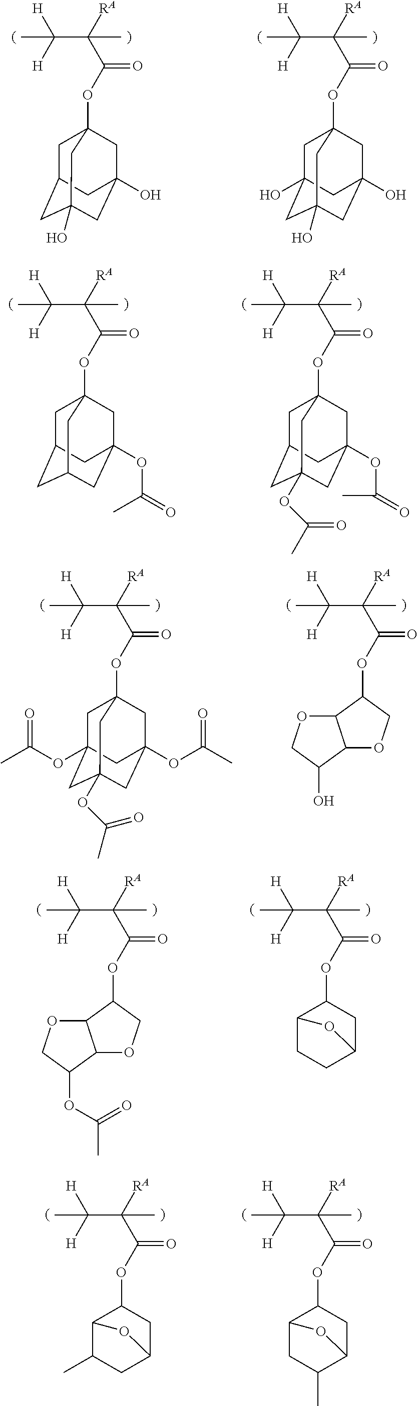

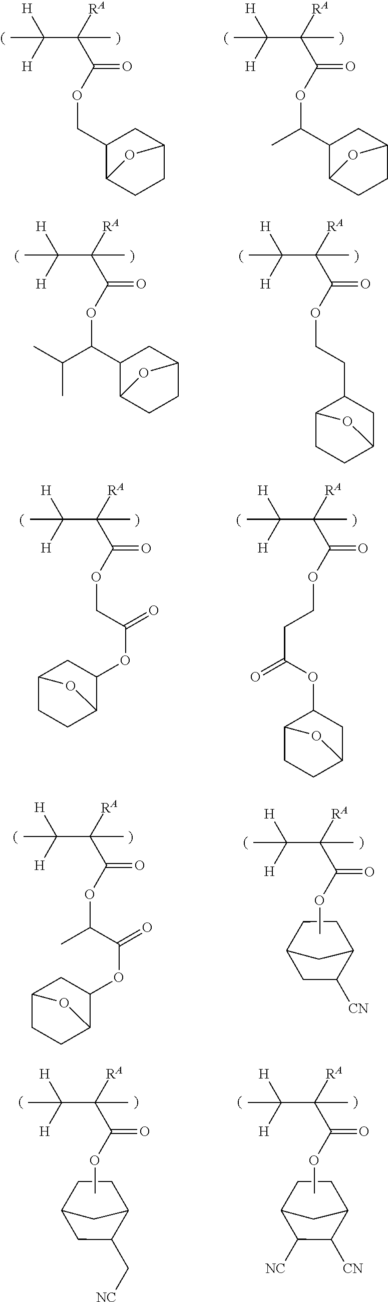

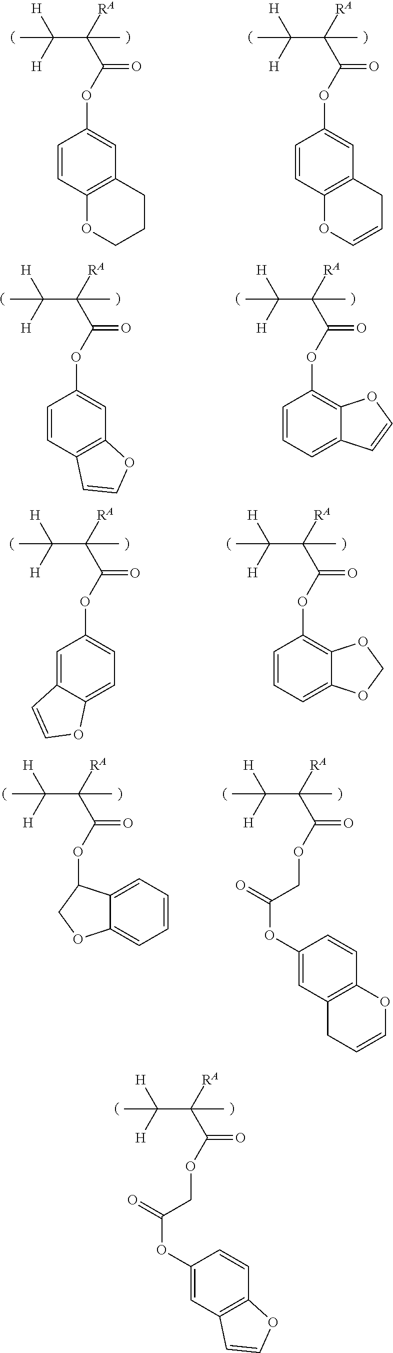

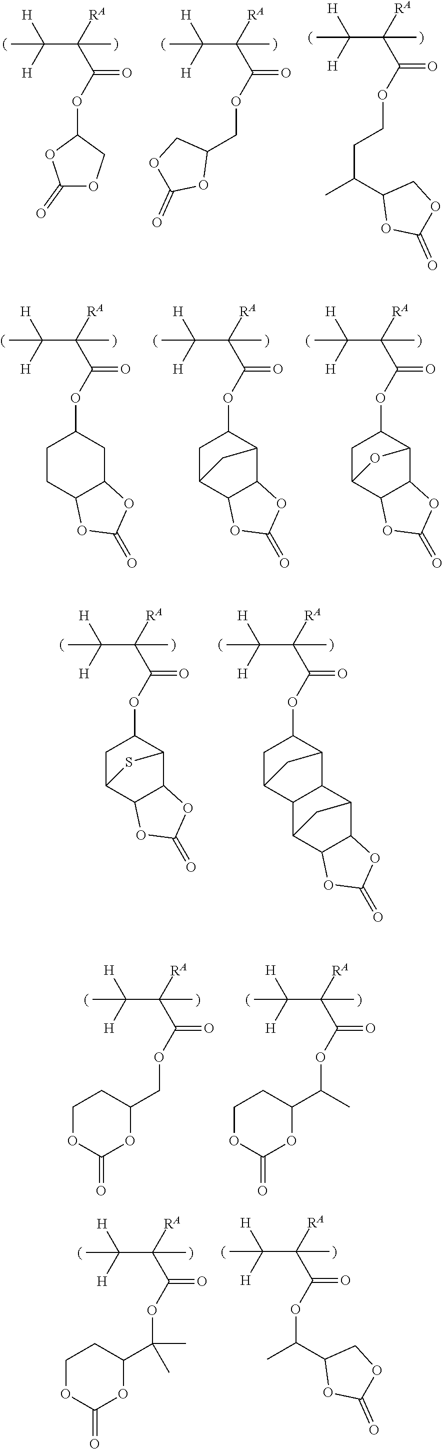

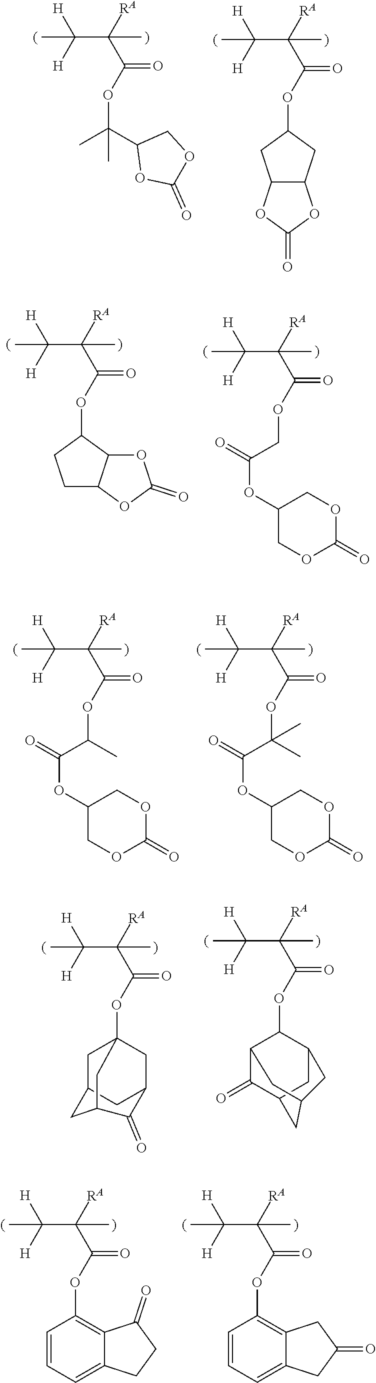

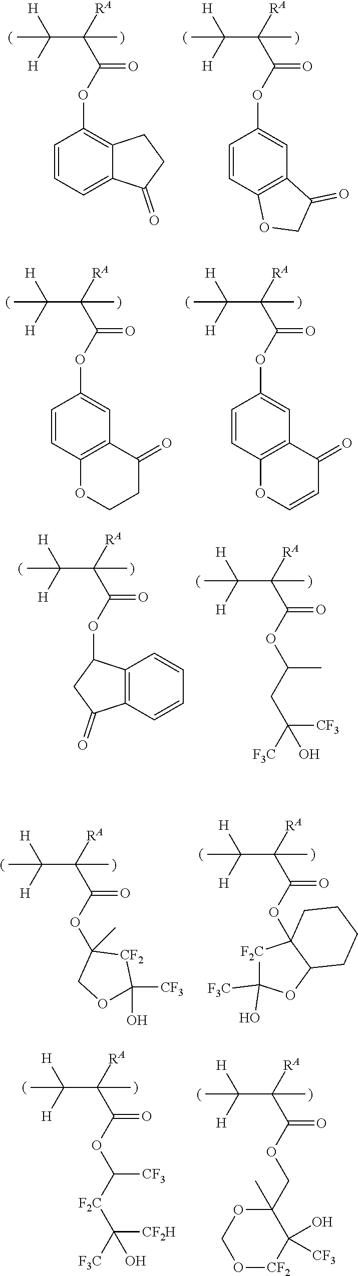

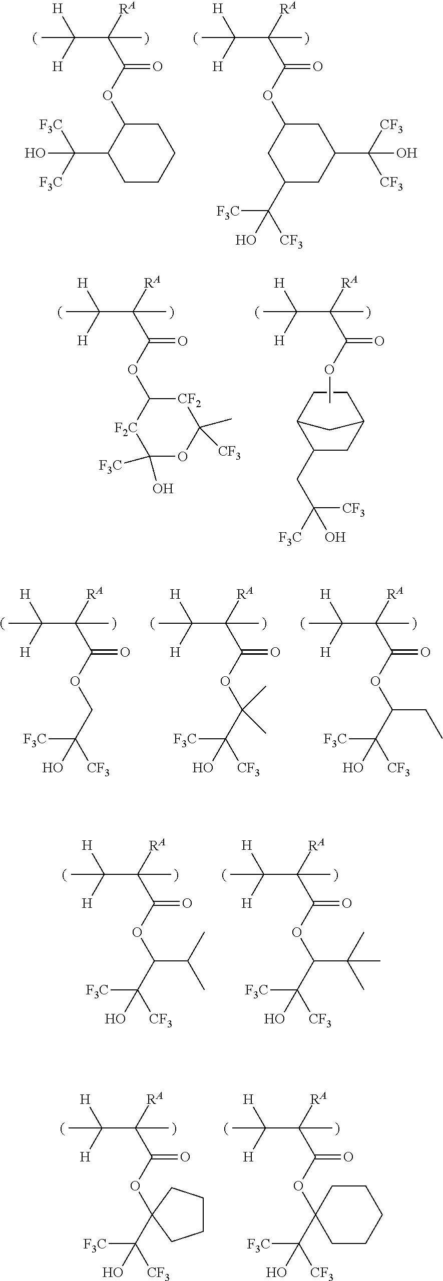

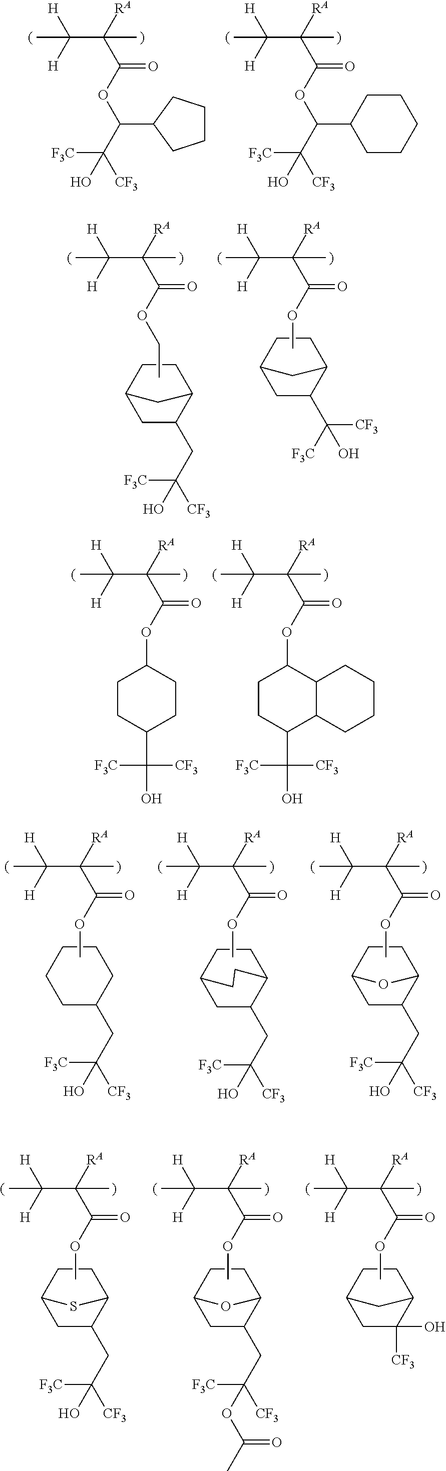

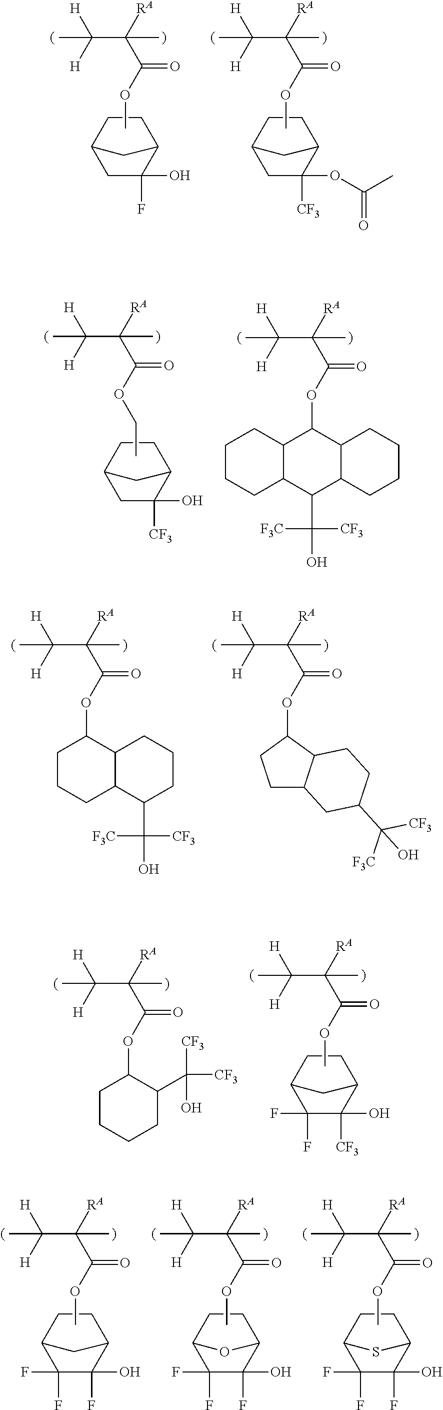

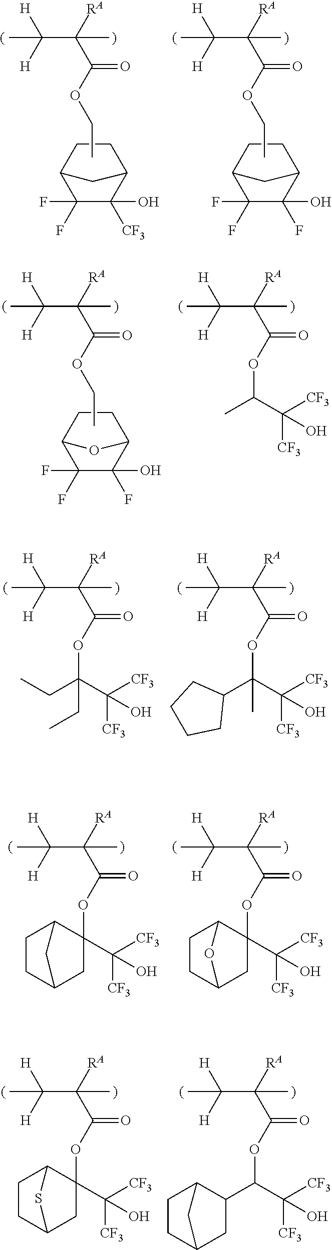

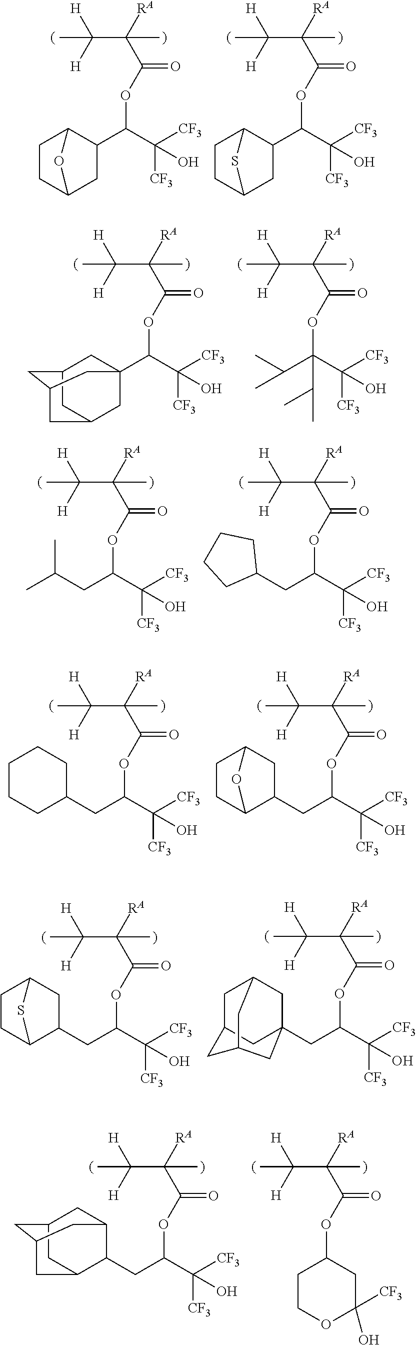

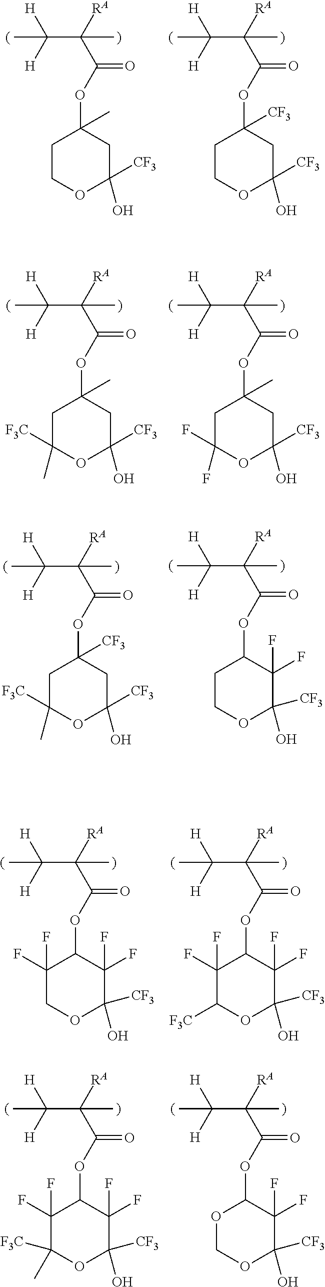

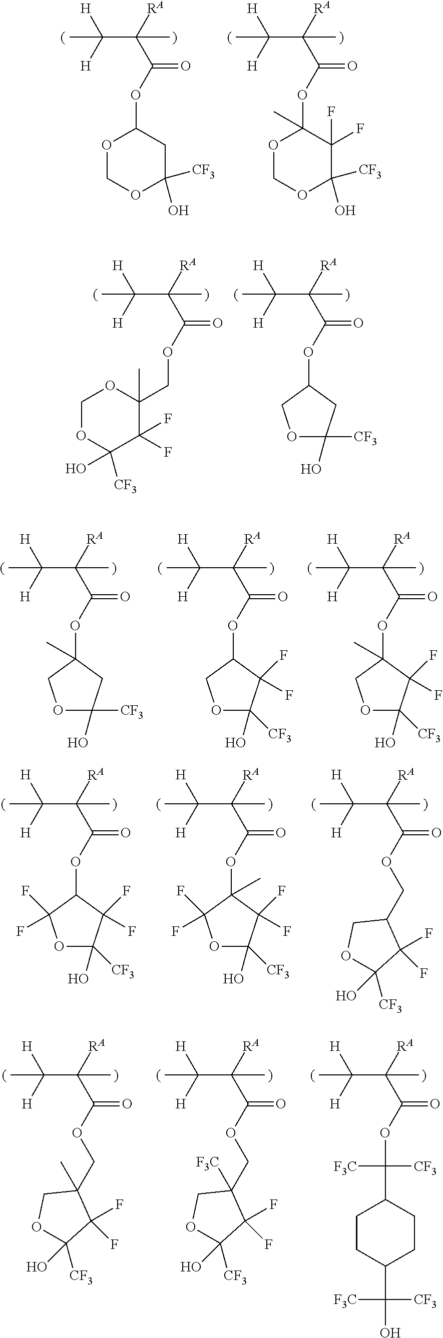

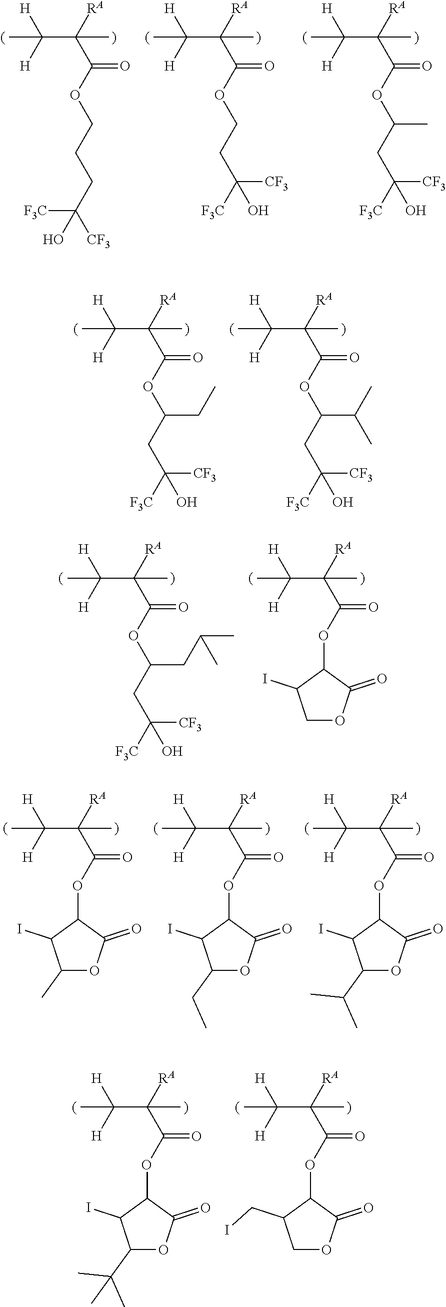

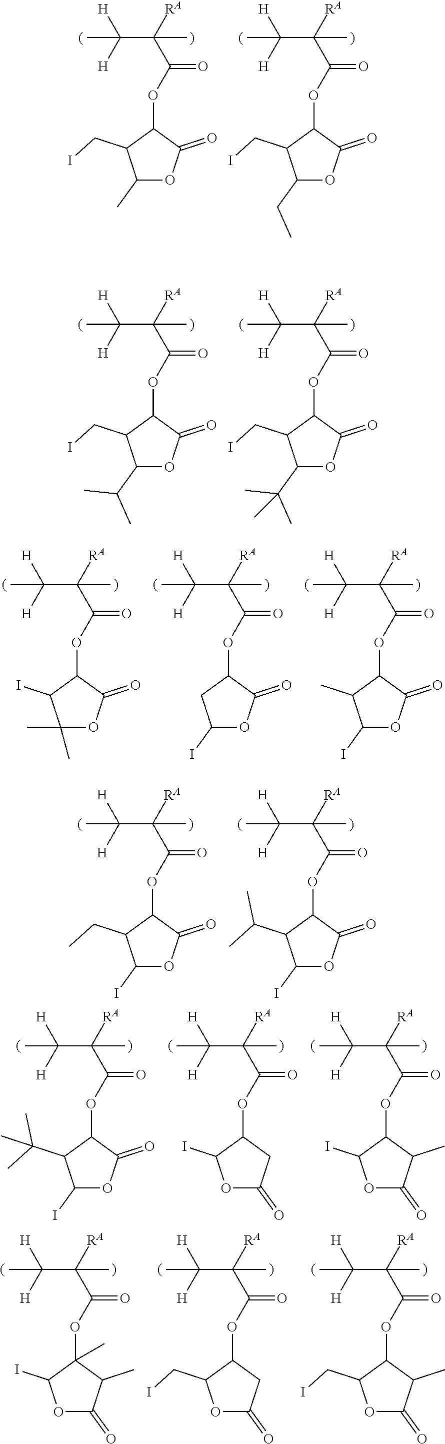

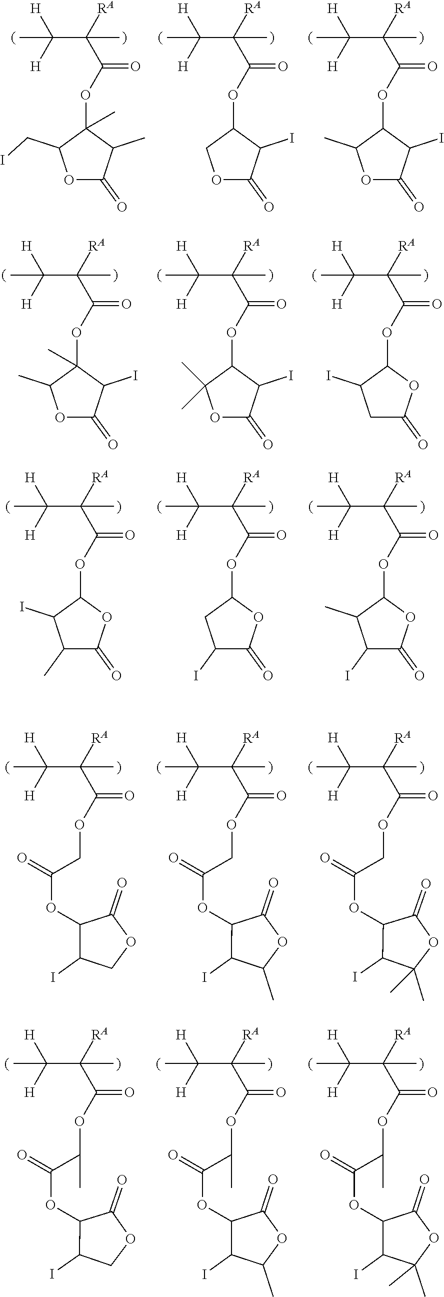

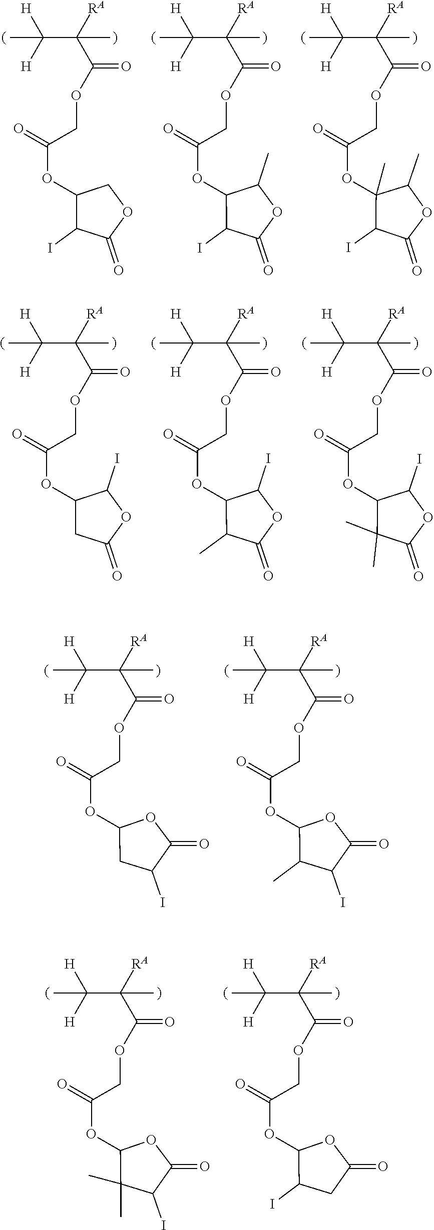

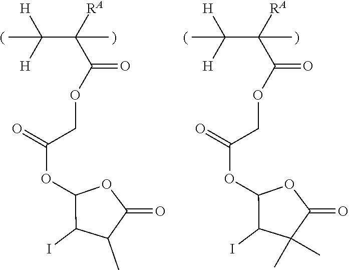

Specific examples of the repeat unit b2 include those shown below, but are not limited thereto. In the following formulae, R^{A} is as defined above.

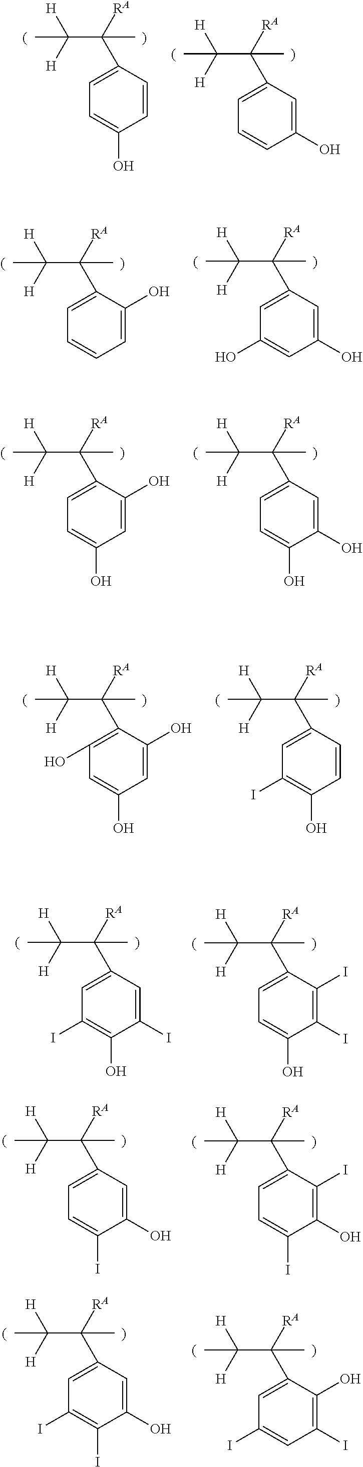

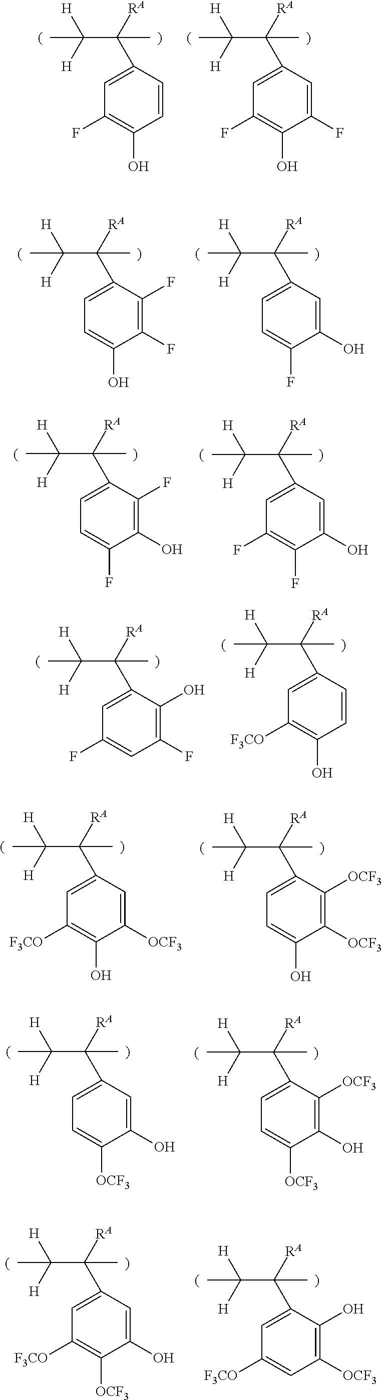

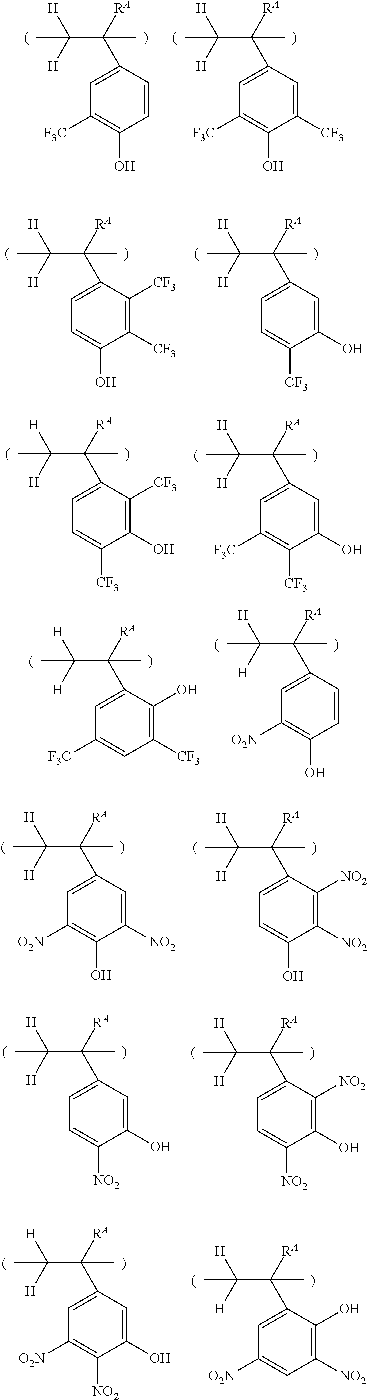

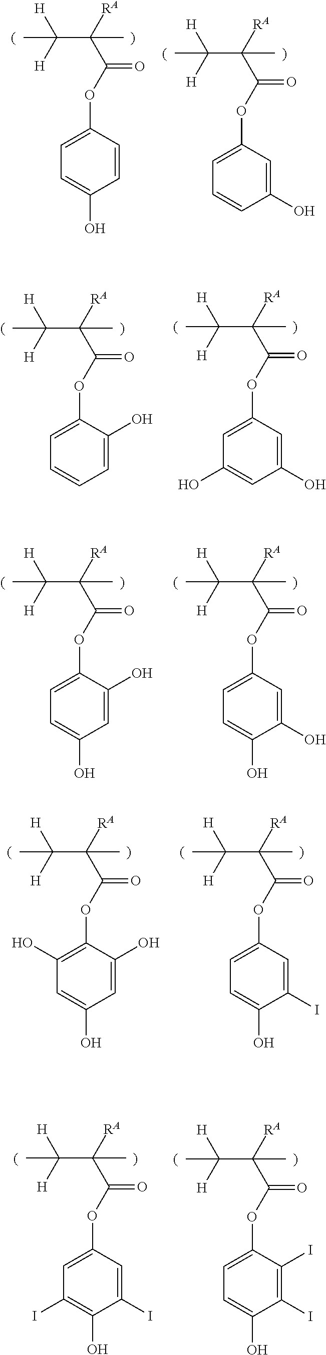

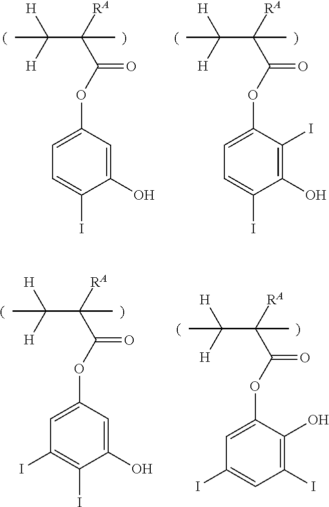

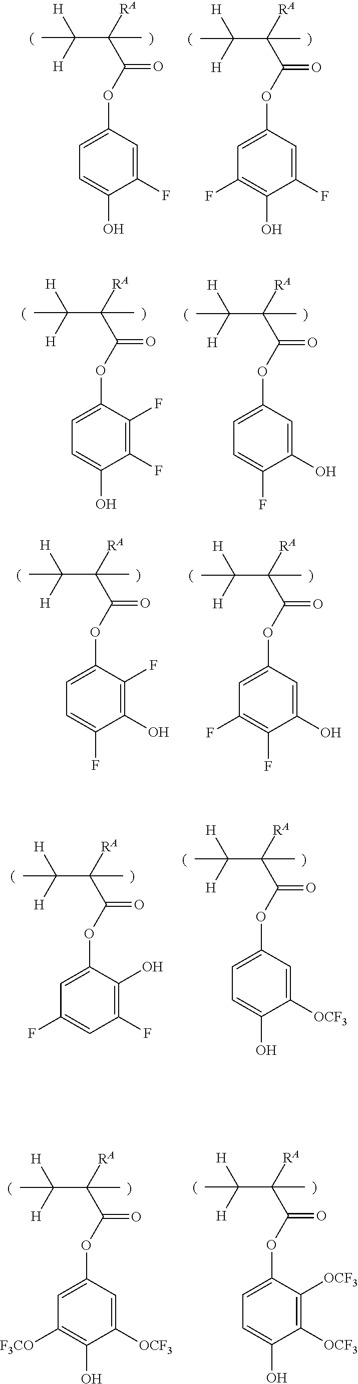

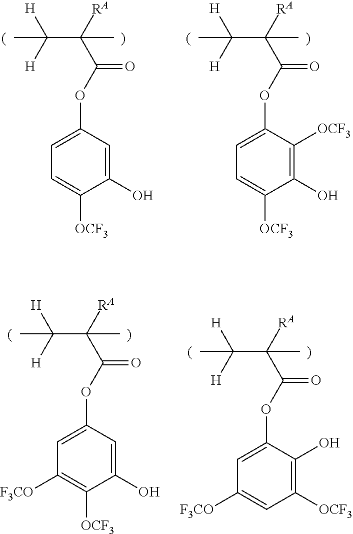

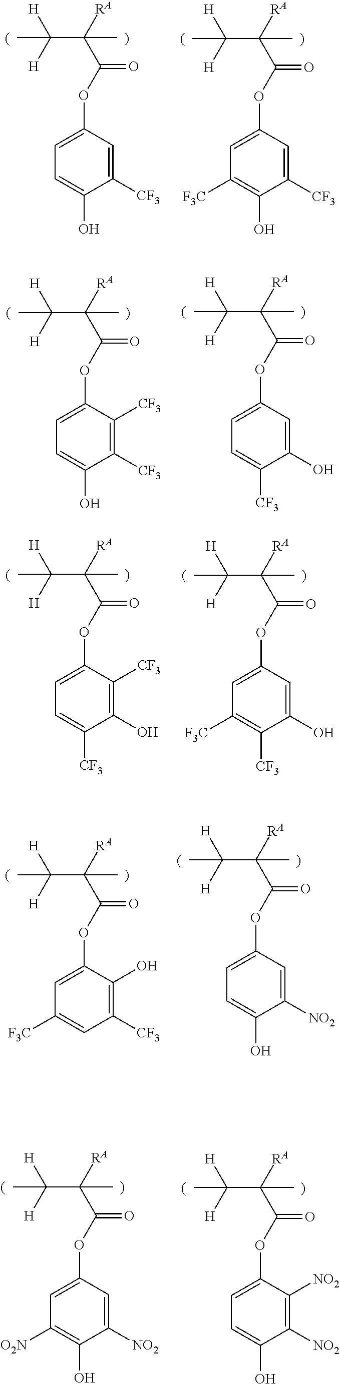

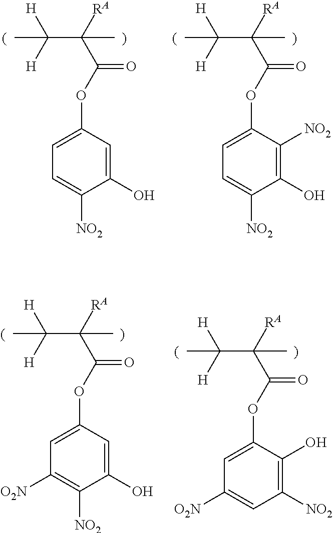

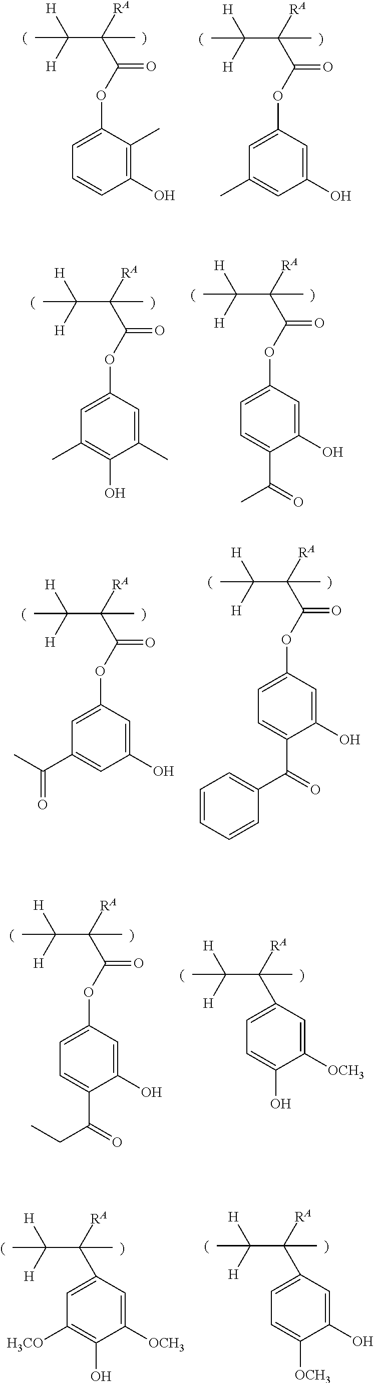

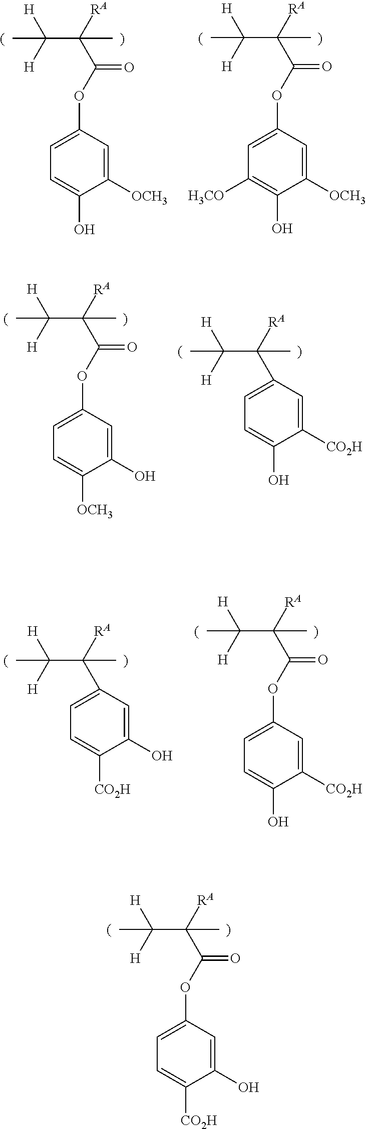

As the repeat unit b1 or b2, particularly a repeat unit having a lactone ring as a polar group is preferred in the case of ArF lithography, and a repeat unit having a phenol site is preferred in the case of KrF lithography, EB lithography, and EUV lithography.

The base polymer may further contain a repeat unit having a structure with a hydroxy group protected with an acid labile group (hereinafter also referred to as repeat unit c). The repeat unit c is not particularly limited as long as it has one or more structures having a hydroxy group protected with a protective group such that the protective group is decomposed to generate a hydroxy group under the action of an acid, and a repeat unit having the formula (c1) is preferred.

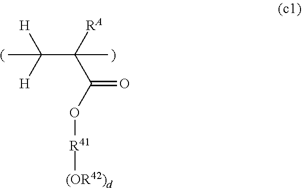

In the formula (c1), R^{A} is as defined above. R⁴¹ is a C₁-C₃₀ (d+1)-valent hydrocarbon group which may contain a heteroatom. R⁵² is an acid labile group. d is an integer of 1 to 4.

In the formula (c1), the acid labile group of R⁴² may be any group that is deprotected by the action of an acid to generate a hydroxy group. The structure of R⁴² is not particularly limited, but is preferably an acetal structure, a ketal structure, an alkoxycarbonyl group, an alkoxymethyl group having the formula (c2), or the like, and particularly preferably an alkoxymethyl group having the formula (c2).

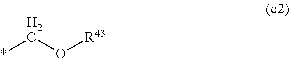

In the formulae, * designates a point of attachment. R⁴³ is a C₁-C₁₅ hydrocarbyl group.

Specific examples of the acid labile group of R⁴², the alkoxymethyl group having the formula (c2), and the repeat unit c are as exemplified for the repeat unit c described in JP-A 2020-111564.

The base polymer may further contain a repeat unit d derived from indene, benzofuran, benzothiophene, acenaphthylene, chromone, coumarin, norbornadiene, or a derivative thereof. Specific examples of the monomer that gives the repeat unit e include those shown below, but are not limited thereto.

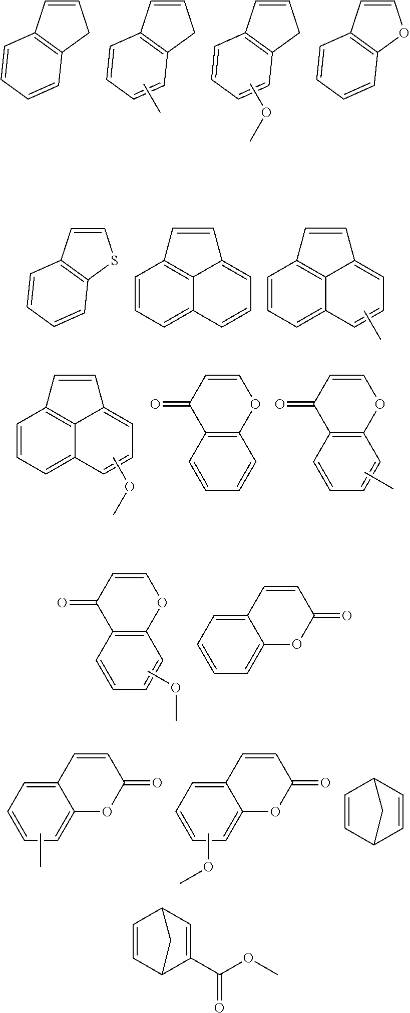

The base polymer may further contain a repeat unit e derived from indane, vinylpyridine, or vinylcarbazole.

In the polymer of the invention, the content ratios of the repeat units A, a1, a2, a3, b1, b2, c, d, and e are preferably 0<A≤0.4, 0<a1≤0.8, 0≤a2≤0.8, 0≤a3≤0.6, 0≤b1≤0.6, 0≤b2≤0.6, 0≤c≤0.5, 0≤d≤0.3, and 0≤e≤0.3, and more preferably 0<A≤0.3, 0<a1≤0.7, 0≤a2≤0.7, 0≤a3≤0.5, 0≤b1≤0.5, 0≤b2≤0.5, 0≤c≤0.3, 0≤d≤0.3, and 0≤e≤0.3, with the proviso that A+a1+a2+a3+b1+b2+c+d+e 1.0.

The polymer preferably has a weight average molecular weight (Mw) of 1,000 to 500,000, and more preferably 3,000 to 100,000. A Mw in the range ensures satisfactory etch resistance and eliminates the risk of resolution being lowered due to a failure to acquire a difference in dissolution rate before and after exposure. In the invention, Mw is a value measured in terms of polystyrene by gel permeation chromatography (GPC) using THF or N,N-dimethylformamide (DMF) as a solvent.

As for the molecular weight distribution (Mw/Mn) of the polymer, since the influence of Mw/Mn tends to become stronger as the pattern rule becomes finer, the polymer preferably has a narrow dispersity (Mw/Mn) of 1.0 to 2.0 in order to provide a resist composition suitable for fine pattern dimensions. A Mw/Mn in the range indicates smaller amounts of lower and higher molecular weight fractions and eliminates the risk of leaving foreign matter on the pattern or degrading the pattern profile after exposure.

The polymer may be synthesized by, for example, adding a radical polymerization initiator and heating and polymerizing a monomer that gives the above-mentioned repeat unit in an organic solvent.

Specific examples of the organic solvent used for the polymerization include toluene, benzene, THF, diethyl ether, dioxane, cyclohexane, cyclopentane, methyl ethyl ketone (MEK), propylene glycol monomethyl ether acetate (PGMEA), and γ-butyrolactone (GBL). Specific examples of the polymerization initiator include 2,2′-azobisisobutyronitrile (AIBN), 2,2′-azobis(2,4-dimethylvaleronitrile), dimethyl-2,2-azobis(2-methylpropionate), 1,1′-azobis(1-acetoxy-1-phenylethane), benzoyl peroxide, and lauroyl peroxide. The amount of the initiator added is preferably 0.01 to 25 mol % based on the total of monomers to be polymerized. The reaction temperature is preferably 50 to 150° C., and more preferably 60 to 100° C. The reaction time is preferably 2 to 24 hours, and more preferably 2 to 12 hours from the aspect of production efficiency.

The polymerization initiator may be added to the monomer solution, which is fed to the reaction vessel, or an initiator solution may be prepared separately from the monomer solution and fed to the reaction vessel independently. Since a polymerization reaction may proceed to form an ultrahigh molecular weight compound due to a radical generated from the initiator in the standby time, it is preferred from the aspect of quality control that the monomer solution and the initiator solution be independently prepared and added dropwise. The acid labile group that has been incorporated in the monomer may be kept as such, or the polymerization may be followed by protection or partial protection. A known chain transfer agent such as dodecylmercaptan or 2-mercaptoethanol may be used for the purpose of adjusting the molecular weight. In that case, the amount of such a chain transfer agent added is preferably 0.01 to 20 mol % based on the total amount of the monomer to be polymerized.

In the case of a monomer containing a hydroxy group, the hydroxy group may be substituted with an acetal group susceptible to deprotection with an acid such as an ethoxyethoxy group during polymerization, and then deprotected with a weak acid and water after polymerization, or may be substituted with an acetyl group, a formyl group, a pivaloyl group, or the like, and then alkali hydrolysis may be performed after polymerization.

When hydroxystyrene or hydroxyvinylnaphthalene is copolymerized, hydroxystyrene or hydroxyvinylnaphthalene and another monomer may be heated and polymerized by adding a radical polymerization initiator in an organic solvent, but acetoxystyrene or acetoxyvinylnaphthalene may be used, and the acetoxy group may be deprotected by alkali hydrolysis after polymerization to form polyhydroxystyrene or hydroxypolyvinylnaphthalene.

As the base during alkaline hydrolysis, ammonia water, triethylamine, or the like can be used. The reaction temperature is preferably −20 to 100° C., and more preferably 0 to 60° C. The reaction time is preferably 0.2 to 100 hours, and more preferably 0.5 to hours.

The amount of each monomer in the monomer solution may be appropriately set, for example, so as to provide the preferred content ratios of the repeat units described above.

As for the polymer obtained by the production method, a reaction solution obtained by a polymerization reaction may be used as a final product, or a powder obtained through a purification step such as a reprecipitation method in which a polymerization liquid is added to a poor solvent to obtain a powder may be handled as a final product. From the aspect of operation efficiency and consistent quality, it is preferred to handle a polymer solution obtained by dissolving the powder obtained by the purification step in a solvent as a final product.

Specific examples of the solvent used at that time include ketones such as cyclohexanone and methyl-2-n-pentyl ketone; alcohols such as 3-methoxybutanol, 3-methyl-3-methoxybutanol, 1-methoxy-2-propanol, and 1-ethoxy-2-propanol; ethers such as propylene glycol monomethyl ether (PGME), ethylene glycol monomethyl ether, propylene glycol monoethyl ether, ethylene glycol monoethyl ether, propylene glycol dimethyl ether, and diethylene glycol dimethyl ether; esters such as PGMEA, propylene glycol monoethyl ether acetate, ethyl lactate, ethyl pyruvate, butyl acetate, methyl 3-methoxypropionate, ethyl 3-ethoxypropionate, tert-butyl acetate, tert-butyl propionate, and propylene glycol mono-tert-butyl ether acetate; lactones such as GBL; alcohols such as diacetone alcohol (DAA); high-boiling alcoholic solvents such as diethylene glycol, propylene glycol, glycerol, 1,4-butanediol, and 1,3-butanediol; and mixed solvents thereof described in paragraphs [0144] to [0145] of JP-A 2008-111103.

The concentration of the polymer in the polymer solution is preferably 0.01 to 30 wt %, and more preferably 0.1 to 20 wt %.

The reaction solution and the polymer solution are preferably filtered through a filter. Filtration is effective for consistent quality because foreign matter and gel which can cause defects can be removed.

Examples of the material of the filter used for the filtration include fluorocarbon, cellulose, nylon, polyester, and hydrocarbon base materials, and in the filtering step of the resist composition, a filter formed of a fluorocarbon base material called Teflon®, a hydrocarbon base material such as polyethylene or polypropylene, or nylon is preferred. The pore size of the filter can be appropriately selected according to the desired cleanliness, but is preferably 100 nm or less, and more preferably 20 nm or less. One type of filter may be used alone or a plurality of filters may be used in combination. Although the filtering method may be single pass of the solution, it is more preferred to perform filtration a plurality of times by circulating the solution. In the polymer preparation process, the filtering step may be performed any times, in any order, but it is preferred to filter the reaction solution after the polymerization reaction, the polymer solution, or both.

### Chemically Amplified Resist Composition

### (a) Base Polymer

The chemically amplified resist composition of the invention contains a base polymer containing the above-mentioned polymer as a component (A).

As the polymer, one type may be used alone or two or more types which are different in compositional ratio, Mw, and/or Mw/Mn may be combined and used. In addition, the base polymer (A) may contain a hydrogenated product of a ring-opening metathesis polymer, and as the hydrogenated product, those described in JP-A 2003-66612 can be used.

### (B) Organic Solvent

The chemically amplified resist composition of the invention may contain an organic solvent as a component (B). The organic solvent (B) is not particularly limited as long as it can dissolve each component described above and each component described below. Specific examples of such an organic solvent include ketones such as cyclopentanone, cyclohexanone and methyl-2-n-pentyl ketone; alcohols such as 3-methoxybutanol, 3-methyl-3-methoxybutanol, 1-methoxy-2-propanol, and 1-ethoxy-2-propanol; ketoalcohols such as DAA; ethers such as PGME, ethylene glycol monomethyl ether, propylene glycol monoethyl ether, ethylene glycol monoethyl ether, propylene glycol dimethyl ether, and diethylene glycol dimethyl ether; esters such as PGMEA, propylene glycol monoethyl ether acetate, ethyl lactate, ethyl pyruvate, butyl acetate, methyl 3-methoxypropionate, ethyl 3-ethoxypropionate, tert-butyl acetate, tert-butyl propionate, and propylene glycol mono-tert-butyl ether acetate; lactones such as GBL; and mixed solvents thereof.

Among these organic solvents, 1-ethoxy-2-propanol, PGMEA, cyclohexanone, GBL, ethyl lactate, DAA, and mixed solvents thereof, in which the solubility of the base polymer of the component (A) is particularly excellent, are preferred.

In the chemically amplified resist composition of the invention, the content of the organic solvent (B) is preferably 200 to 7,000 parts by weight, and more preferably 400 to 5,000 parts by weight per 80 parts by weight of the base polymer (A). As the organic solvent (B), one type may be used alone or two or more types may be mixed and used.

### (C) Quencher

The chemically amplified resist composition of the invention may contain a quencher as a component (C). In the invention, the quencher is a material for forming a desired pattern by trapping an acid generated from a photoacid generator in a chemically amplified resist composition to prevent diffusion to an unexposed portion.

Specific examples of the quencher (C) include an onium salt having the formula (1) or (2).

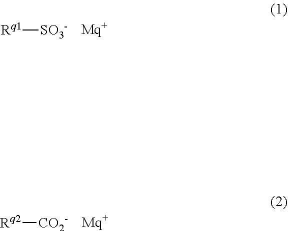

In the formula (1), R^{q1} is a hydrogen atom or a C₁-C₄₀ hydrocarbyl group which may contain a heteroatom, exclusive of a group in which a hydrogen atom attached to a carbon atom at the α-position of a sulfo group is substituted is substituted with a fluorine atom or a fluoroalkyl group. In the formula (2), R^{q2} is a hydrogen atom or a C₁-C₄₀ hydrocarbyl group which may contain a heteroatom.

Specific examples of the C₁-C₄₀ hydrocarbyl group of R^{q1} include C₁-C₄₀ alkyl groups such as a methyl group, an ethyl group, a n-propyl group, an isopropyl group, a n-butyl group, an isobutyl group, a sec-butyl group, a tert-butyl group, a n-pentyl group, a tert-pentyl group, a n-hexyl group, a n-octyl group, a 2-ethylhexyl group, a n-nonyl group, and a n-decyl group; C₃-C₄₀ cyclic saturated hydrocarbyl groups such as a cyclopentyl group, a cyclohexyl group, a cyclopentylmethyl group, a cyclopentylethyl group, a cyclopentylbutyl group, a cyclohexylmethyl group, a cyclohexylethyl group, a cyclohexylbutyl group, a norbornyl group, a tricyclo[5.2.1.0^{2,6}]decyl group, and an adamantyl group; and C₆-C₄₀ aryl groups such as a phenyl group, a naphthyl group, and an anthracenyl group. Some or all hydrogen atoms in the hydrocarbyl group may be substituted with a group containing a heteroatom such as an oxygen atom, a sulfur atom, a nitrogen atom, or a halogen atom, and some —CH₂— in the hydrocarbyl group may be substituted with a group containing a heteroatom such as an oxygen atom, a sulfur atom, or a nitrogen atom, so that the hydrocarbyl group may contain a hydroxy group, a fluorine atom, a chlorine atom, a bromine atom, an iodine atom, a cyano group, a carbonyl group, an ether bond, an ester bond, a sulfonate ester bond, a carbonate bond, a lactone ring, a sultone ring, a carboxylic anhydride (—C(═O)—O—C(═O)—), a haloalkyl group, or the like.

Specific examples of the hydrocarbyl group of R^{q2} include, in addition to the substituents exemplified as specific examples of R^{q1}, fluorinated saturated hydrocarbyl groups such as a trifluoromethyl group and a trifluoroethyl group, and fluorinated aryl groups such as a pentafluorophenyl group and a 4-trifluoromethylphenyl group.

Specific examples of the anion of the onium salt having the formula (1) include those shown below, but are not limited thereto.

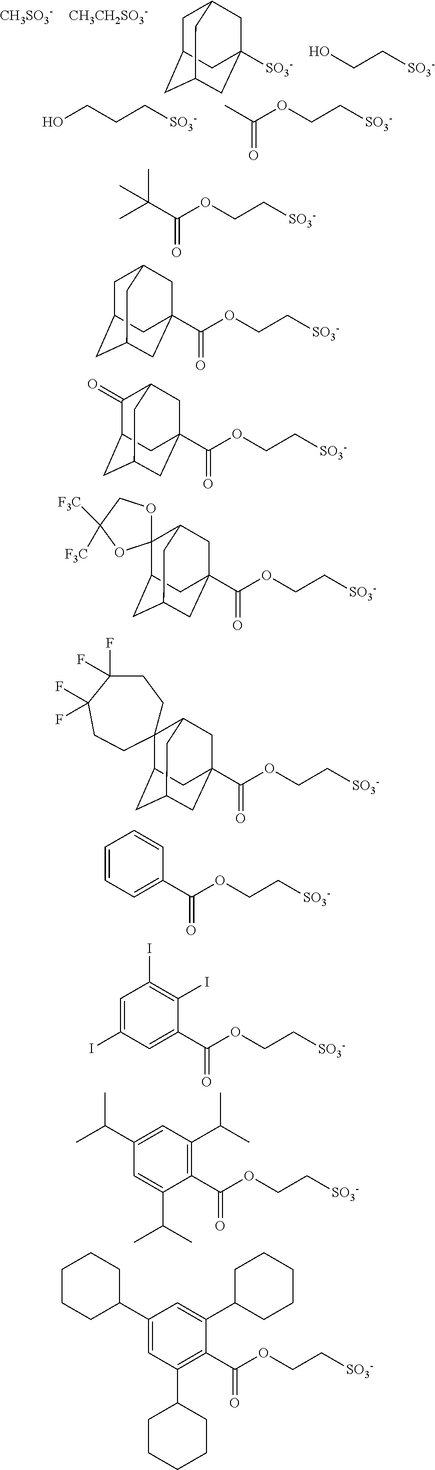

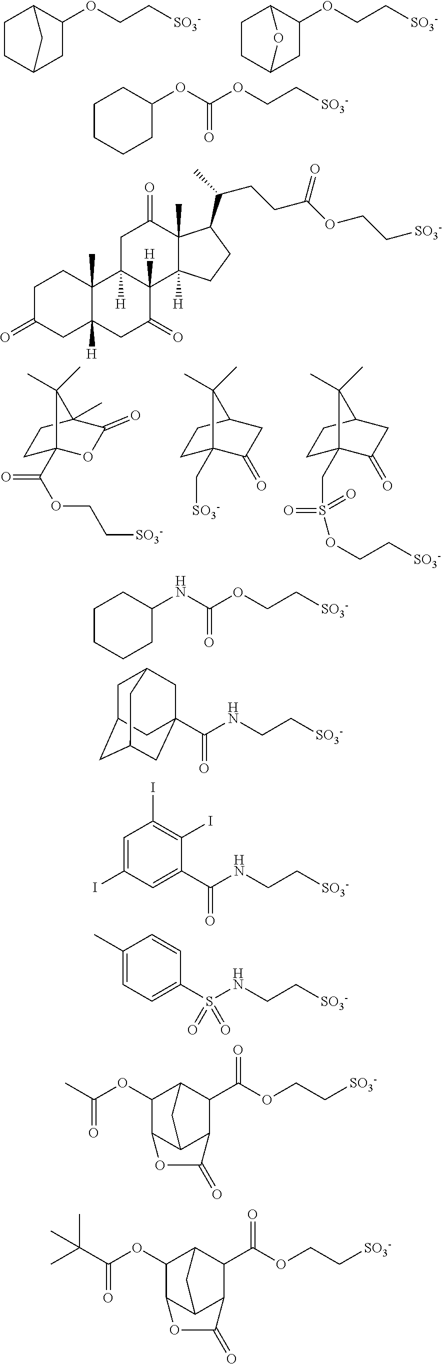

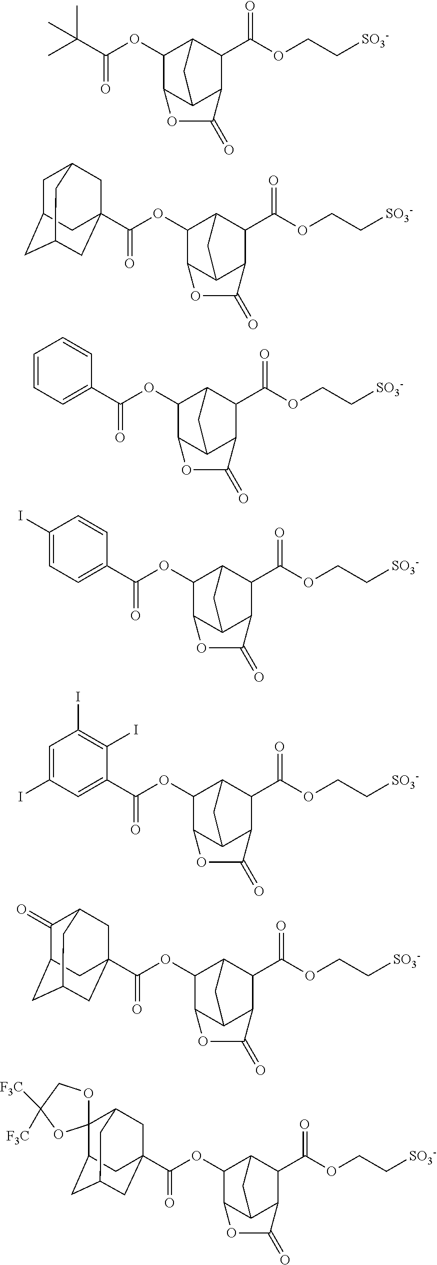

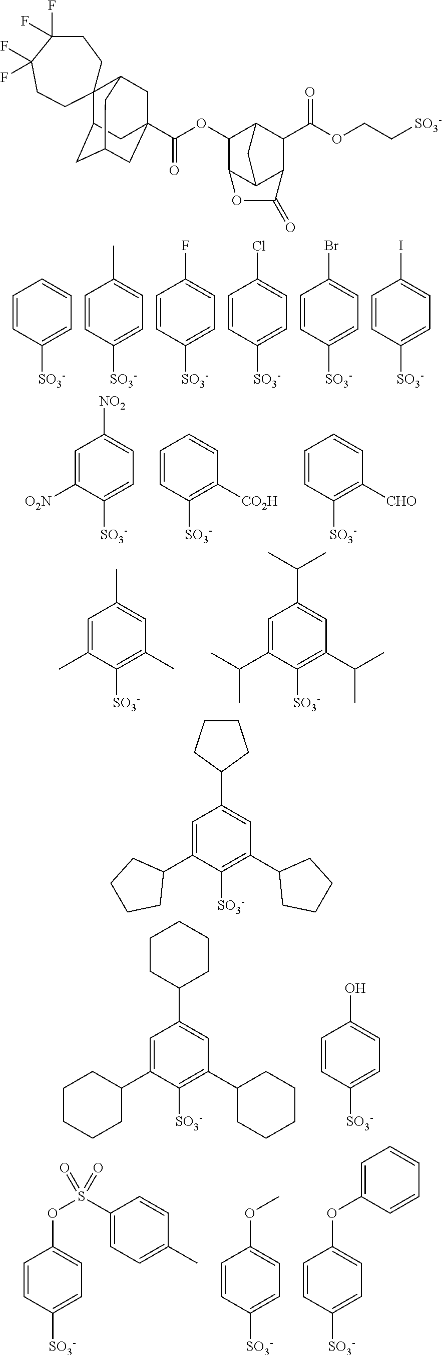

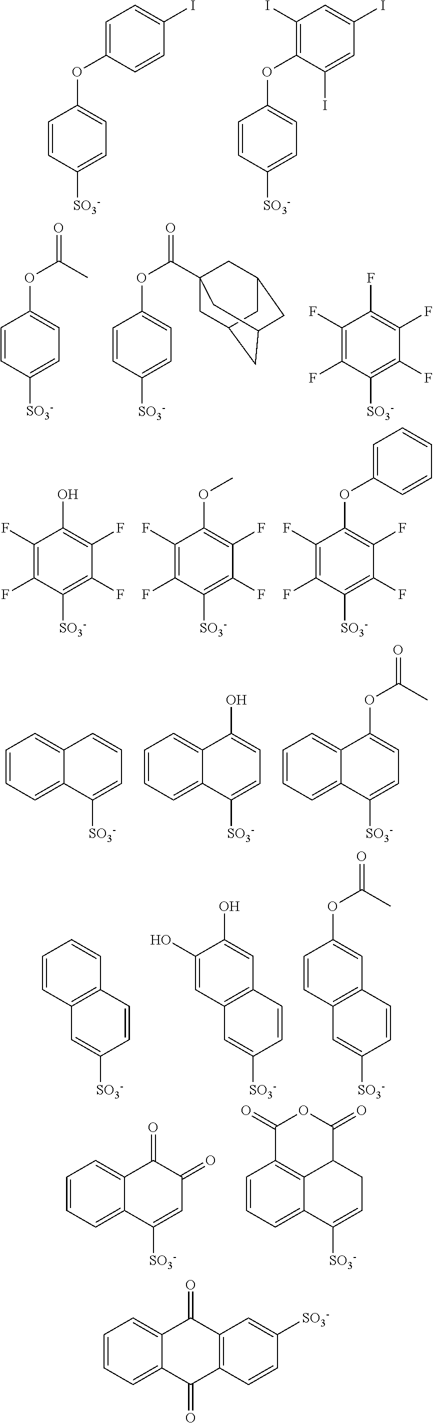

Specific examples of the anion of the onium salt having the formula (2) include those shown below, but are not limited thereto.

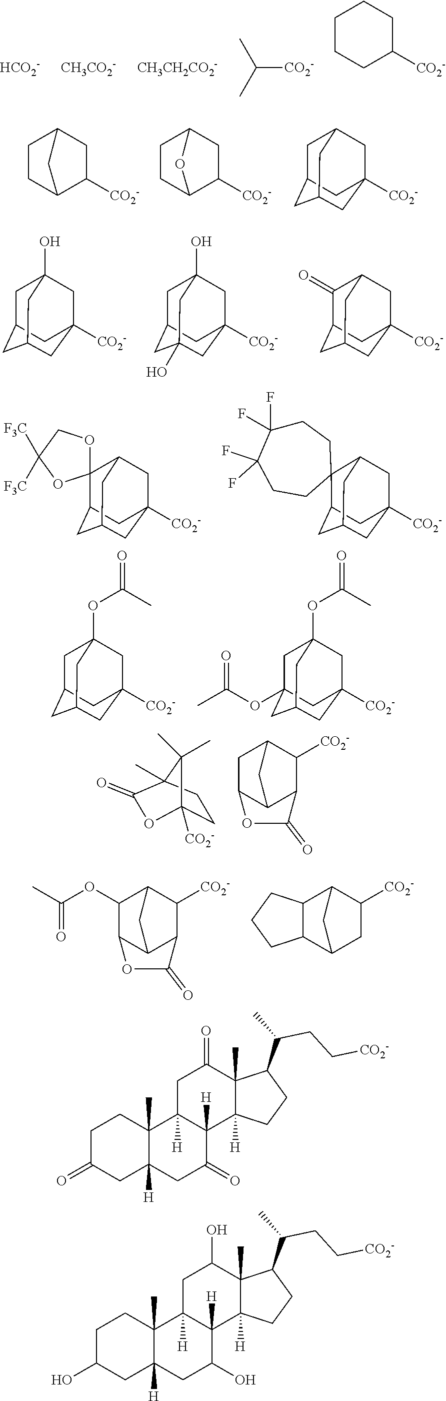

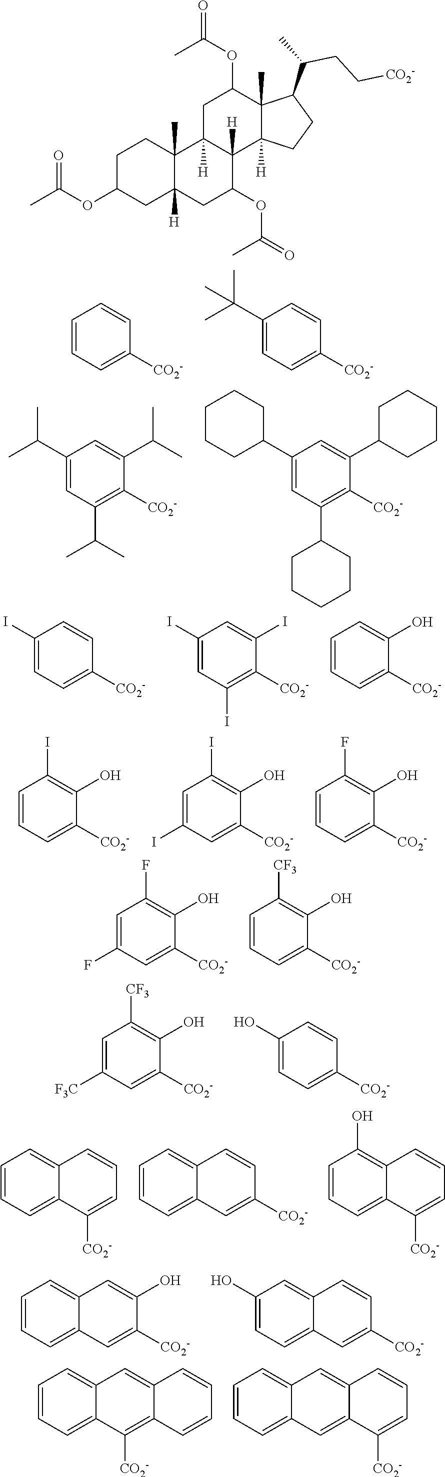

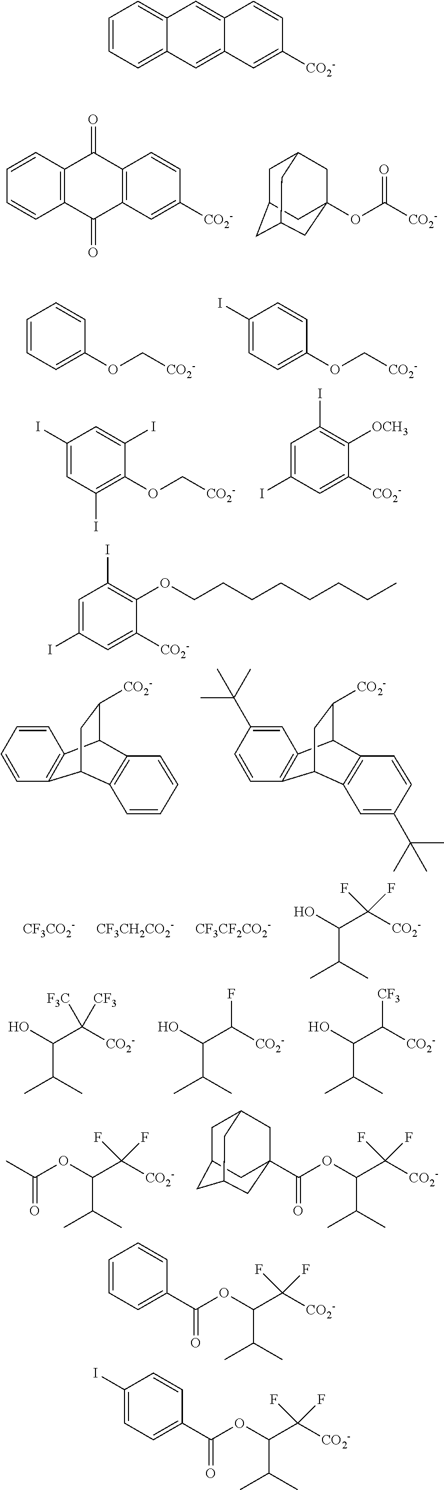

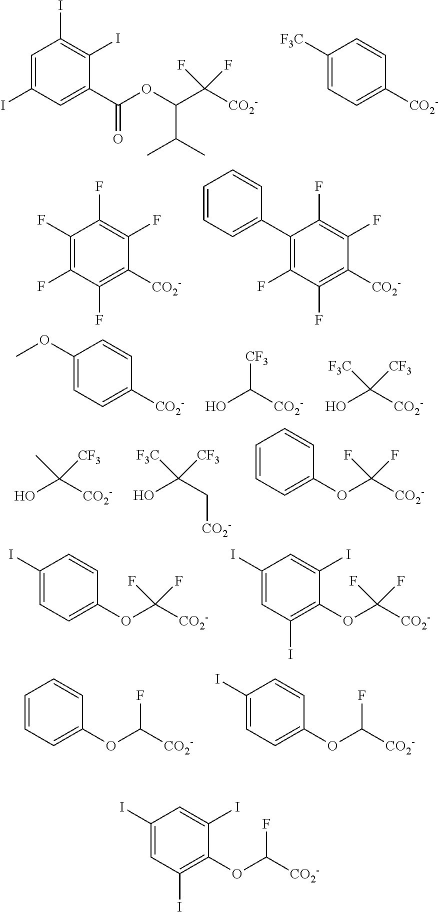

In the formulae (1) and (2), Mq⁺ is an onium cation. Examples of the onium cation include a sulfonium cation, an iodonium cation, and an ammonium cation. Specific examples of the sulfonium cation include those exemplified as specific examples of the sulfonium cation in the formula (A) and those described in paragraphs [0102] to [0125] of JP-A 2024-3744, but are not limited thereto. Specific examples of the iodonium cation include those described in paragraph [0181] of JP-A 2024-259, but are not limited thereto. Specific examples of the ammonium cation include ammonium cations having the formula (am-1).

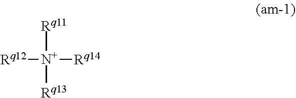

In the formula (am-1), R^{q11} to R^{q14} are each independently a C₁-C₄₀ hydrocarbyl group which may contain a heteroatom. R^{q11} and R^{g12} may bond together to form a ring with a nitrogen atom to which they are attached. Specific examples of the hydrocarbyl group are as exemplified above for the hydrocarbyl group of R¹ in the description of the formula (A).

Specific examples of the ammonium cation having the formula (am-1) include those shown below, but are not limited thereto.

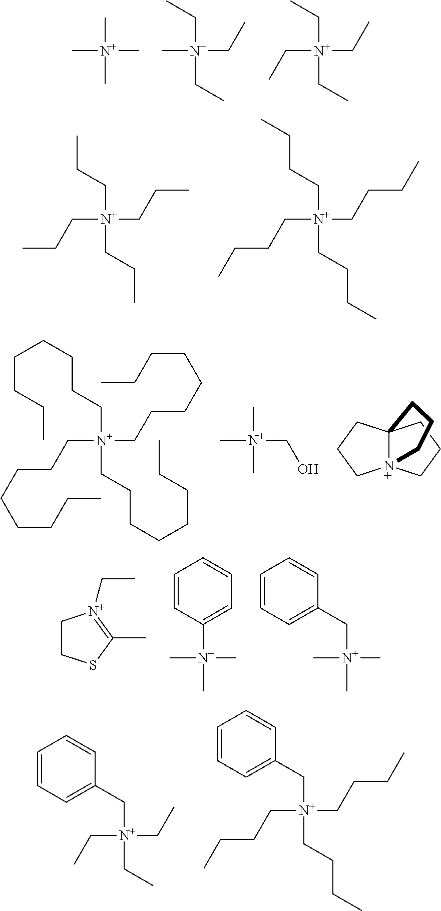

Specific examples of the onium salt having the formula (1) or (2) include arbitrary combinations of the anion with the cation described above. These onium salts are easily prepared by an ion exchange reaction using a known organic chemical method. With respect to the ion exchange reaction, for example, JP-A 2007-145797 can be referred to.

The onium salt having the formula (1) or (2) acts as a quencher in the chemically amplified resist composition of the invention. This is because each counter anion of the onium salt is a conjugate base of a weak acid. The weak acid as used herein means an acid having an acidity insufficient to deprotect an acid labile group of an acid labile group-containing unit in the base polymer. The onium salt having the formula (1) or (2) functions as a quencher when used in combination with an onium salt photoacid generator having a strong acid conjugated base such as sulfonic acid fluorinated at the α-position as the counter anion. That is, when an onium salt that generates a strong acid such as sulfonic acid fluorinated at the α-position and an onium salt that generates a weak acid such as sulfonic acid or carboxylic acid that is not fluorinated are mixed and used, if the strong acid generated from the photoacid generator by irradiation with high-energy radiation collides with an unreacted onium salt having a weak acid anion, the weak acid is released by salt exchange to produce an onium salt having a strong acid anion. In this process, the strong acid is exchanged with the weak acid having a lower catalysis, so that the acid is apparently deactivated and acid diffusion can be controlled.

As the quencher (C), an onium salt having sulfonium cation and phenoxide anion sites in a common molecule described in JP 6848776, an onium salt having sulfonium cation and carboxylate anion sites in a common molecule described in JP 6583136 and JP-A 2020-200311, and an onium salt having iodonium cation and carboxylate anion sites in a common molecule described in JP 6274755 can also be used.

Here, when the photoacid generator that generates a strong acid is an onium salt, as described above, the strong acid generated by irradiation with high-energy radiation can be exchanged with a weak acid, but it is considered difficult for a weak acid generated by irradiation with high-energy radiation to collide with an unreacted onium salt that generates a strong acid to induce salt exchange. This is due to the phenomenon that an onium cation easily forms an ion pair with a stronger acid anion.

When the chemically amplified resist composition of the invention contains the onium salt having the formula (1) or (2) as the quencher (C), the content thereof is preferably 0.1 to 20 parts by weight, and more preferably 0.1 to 10 parts by weight per 80 parts by weight of the base polymer (A). The content of the onium salt quencher of the component (C) in the above range is preferred because a satisfactory resolution is available without a substantial lowering of the sensitivity. As the onium salt having the formula (1) or (2), one type may be used alone or two or more types may be combined and used.

The chemically amplified resist composition of the invention may contain a nitrogen-containing compound as the quencher (C). Specific examples of the nitrogen-containing compound as the component (C) include primary, secondary, or tertiary amine compounds described in paragraphs [0146] to [0164] of JP-A 2008-111103, particularly amine compounds having a hydroxy group, an ether bond, an ester bond, a lactone ring, a cyano group, or a sulfonate ester bond. Additional examples include a compound obtained by protecting a primary or secondary amine with a carbamate group described in JP 3790649.

A sulfonic acid sulfonium salt having a nitrogen-containing substituent may also be used as the nitrogen-containing compound. Such a compound functions as a quencher in an unexposed portion, and as a so-called photo-degradable base that loses the quencher ability due to neutralization with the acid generated by itself in an exposed portion. Use of a photo-degradable base can further enhance the contrast between the exposed portion and the unexposed portion. With respect to the photo-degradable base, for example, JP-A 2009-109595, JP-A 2012-46501, and the like can be referred to.

When the chemically amplified resist composition of the invention contains a nitrogen-containing compound as the quencher (C), the content thereof is preferably 0.001 to 12 parts by weight, and more preferably 0.01 to 8 parts by weight per 80 parts by weight of the base polymer (A). As the nitrogen-containing compound, one type may be used alone or two or more types may be combined and used.

### (D) Photoacid Generator

The chemically amplified resist composition of the invention may contain a photoacid generator other than the component (A) (hereinafter also referred to as another photoacid generator) as the component (D). The another photoacid generator is not particularly limited as long as it is a compound that generates an acid by irradiation with high-energy radiation. Preferred examples of the another photoacid generator include photoacid generators having the formula (3) or (4).

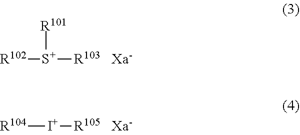

In the formula (3), R¹⁰¹ to R¹⁰⁵ are each independently a halogen atom or a C₁-C₂₀ hydrocarbyl group which may contain a heteroatom. Any two of R¹⁰¹, R¹⁰², and R¹⁰³ may bond together to form a ring with a sulfur atom to which they are attached.

Specific examples of the cation of the sulfonium salt having the formula (3) include those exemplified as specific examples of the sulfonium cation in the formula (A) and those described in paragraphs [0102] to [0125] of JP-A 2024-3744, but are not limited thereto. Specific examples of the cation of the iodonium salt having the formula (4) include those described in paragraph [0181] of JP-A 2024-259, but are not limited thereto.

In the formulae (3) and (4), Xa⁻ is a strong acid anion. Examples of the strong acid anion include anions having the formulae (Xa-1) to (Xa-4).

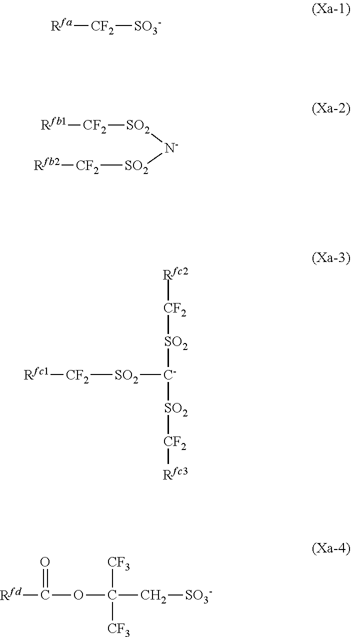

In the formula (Xa-1), R^{fa} is a fluorine atom or a C₁-C₄₀ hydrocarbyl group which may contain a heteroatom. The hydrocarbyl group may be saturated or unsaturated and straight, branched, or cyclic. Specific examples thereof are as exemplified later for the hydrocarbyl group of R^{fa1} in the formula (Xa-1-1).

As the anion having the formula (Xa-1), an anion having the formula (Xa-1-1) is preferred.

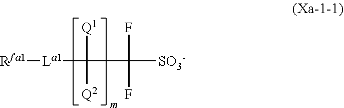

In the formula (Xa-1-1), Q¹ and Q² are each independently a hydrogen atom, a fluorine atom, or a C₁-C₆ fluorinated saturated hydrocarbyl group, but at least one of them is preferably a trifluoromethyl group for improving the solvent solubility. m is 0, 1, 2, 3, or 4, and is particularly preferably 1. R^{fa1} is a C₁-C₃₅ hydrocarbyl group which may contain a heteroatom, The heteroatom is preferably an oxygen atom, a nitrogen atom, a sulfur atom, a halogen atom, or the like, and more preferably an oxygen atom. As the hydrocarbyl group, a C₆-C₃₀ hydrocarbyl group is particularly preferred from the aspect of obtaining high resolution in fine pattern formation.

In the formula (Xa-1-1), the C₁-C₃₅ hydrocarbyl group of R¹¹ may be saturated or unsaturated and straight, branched, or cyclic. Specific examples thereof include C₁-C₃₅ alkyl groups such as a methyl group, an ethyl group, a n-propyl group, an isopropyl group, a n-butyl group, an isobutyl group, a sec-butyl group, a tert-butyl group, a pentyl group, a neopentyl group, a hexyl group, a heptyl group, a 2-ethylhexyl group, a nonyl group, an undecyl group, a tridecyl group, a pentadecyl group, a heptadecyl group, and an icosyl group; C₃-C₃₅ cyclic saturated hydrocarbyl groups such as a cyclopentyl group, a cyclohexyl group, a 1-adamantyl group, a 2-adamantyl group, a 1-adamantylmethyl group, a norbornyl group, a norbornylmethyl group, a tricyclodecyl group, a tetracyclododecyl group, a tetracyclododecylmethyl group, and a dicyclohexylmethyl group; C₂-C₃₅ unsaturated aliphatic hydrocarbyl groups such as a 2-propenyl group and a 3-cyclohexenyl group; C₆-C₃₅ aryl groups such as a phenyl group, a 1-naphthyl group, a 2-naphthyl group, and a 9-fluorenyl group; C₇-C₃₅ aralkyl groups such as a benzyl group and a diphenylmethyl group; and a group obtained by combining these groups.

Some or all hydrogen atoms in the hydrocarbyl group may be substituted with a group containing a heteroatom such as an oxygen atom, a sulfur atom, a nitrogen atom, or a halogen atom, and some —CH₂— in the hydrocarbyl group may be substituted with a group containing a heteroatom such as an oxygen atom, a sulfur atom, or a nitrogen atom, so that the hydrocarbyl group may contain a hydroxy group, a fluorine atom, a chlorine atom, a bromine atom, an iodine atom, a cyano group, a nitro group, a carbonyl group, an ether bond, an ester bond, a sulfonate ester bond, a carbonate bond, a lactone ring, a sultone ring, a carboxylic anhydride (—C(═O)—O—C(═O)—), a haloalkyl group, or the like. Specific examples of the hydrocarbyl group containing a heteroatom include a tetrahydrofuryl group, a methoxymethyl group, an ethoxymethyl group, a methylthiomethyl group, an acetamidomethyl group, a trifluoroethyl group, a (2-methoxyethoxy)methyl group, an acetoxymethyl group, a 2-carboxy-1-cyclohexyl group, a 2-oxopropyl group, a 4-oxo-1-adamantyl group, and a 3-oxocyclohexyl group.

In the formula (Xa-1-1), L^{a1} is a single bond, an ether bond, an ester bond, a sulfonate ester bond, a carbonate bond, or a carbamate bond, but is preferably an ether bond or an ester bond, and more preferably an ester bond from the aspect of synthesis.

Specific examples of the anion having the formula (Xa-1) include those shown below, but are not limited thereto. In the following formulae, Q¹ is as defined above, and Ac is an acetyl group.

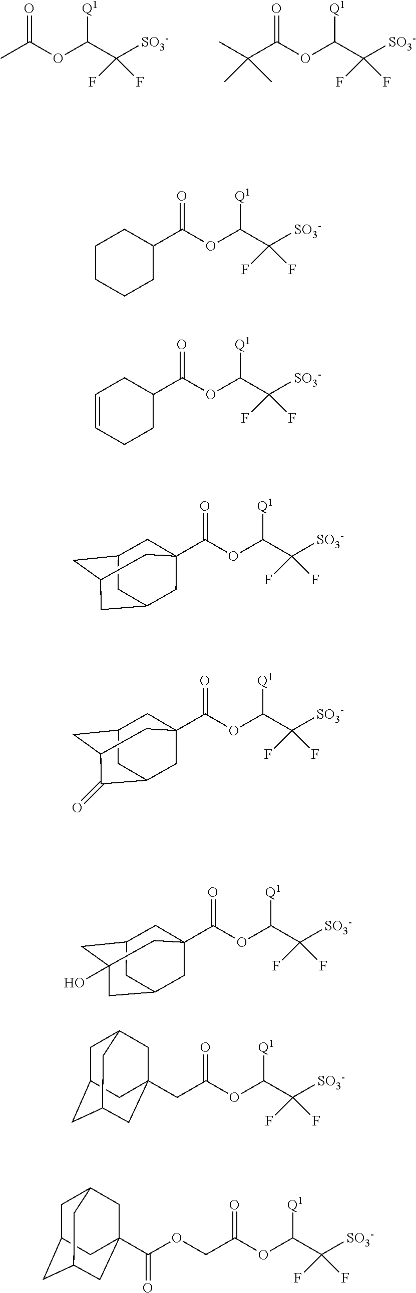

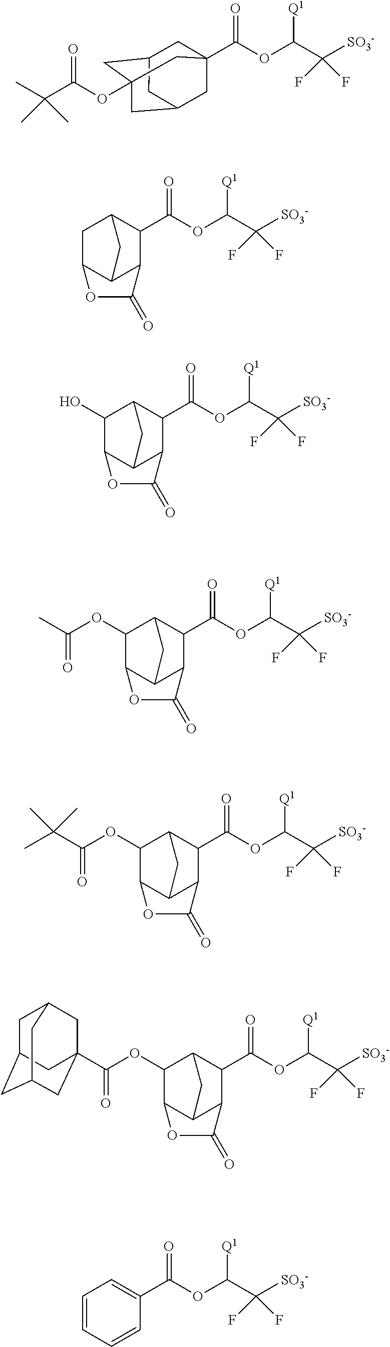

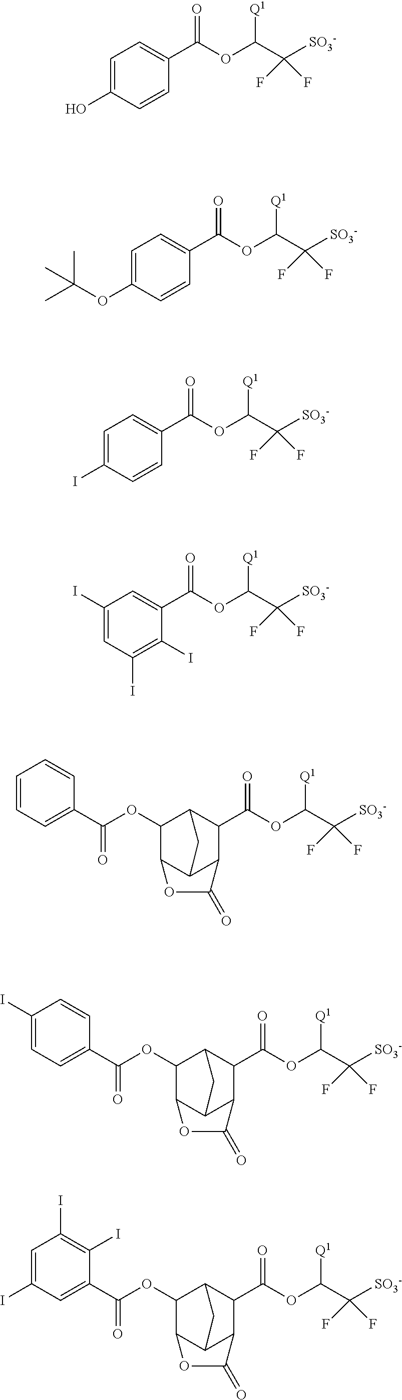

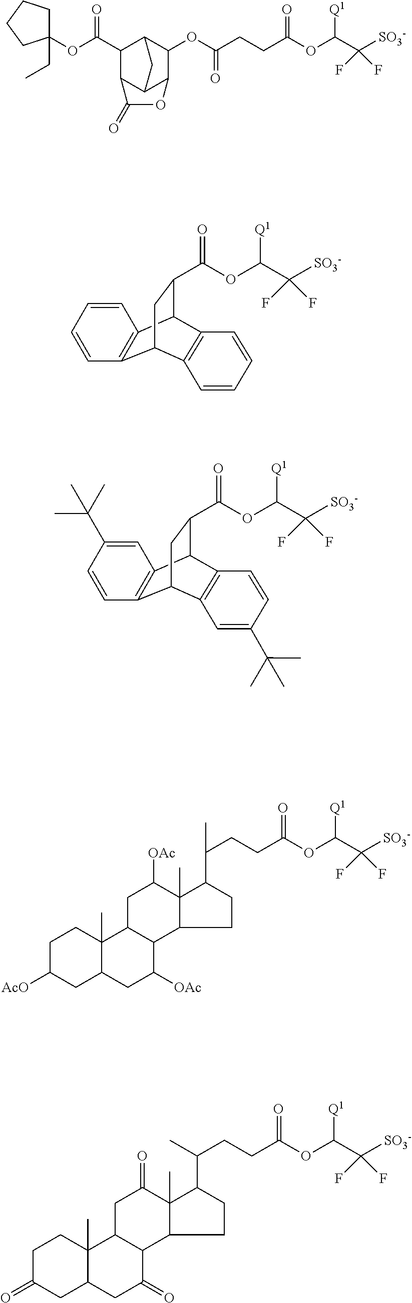

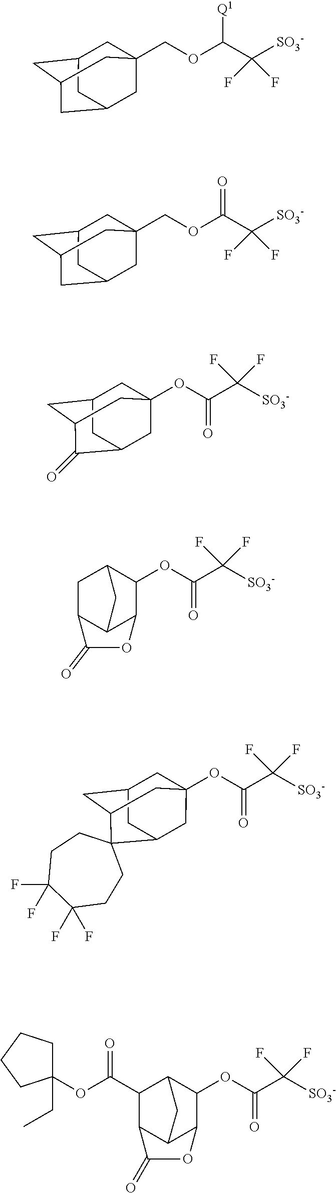

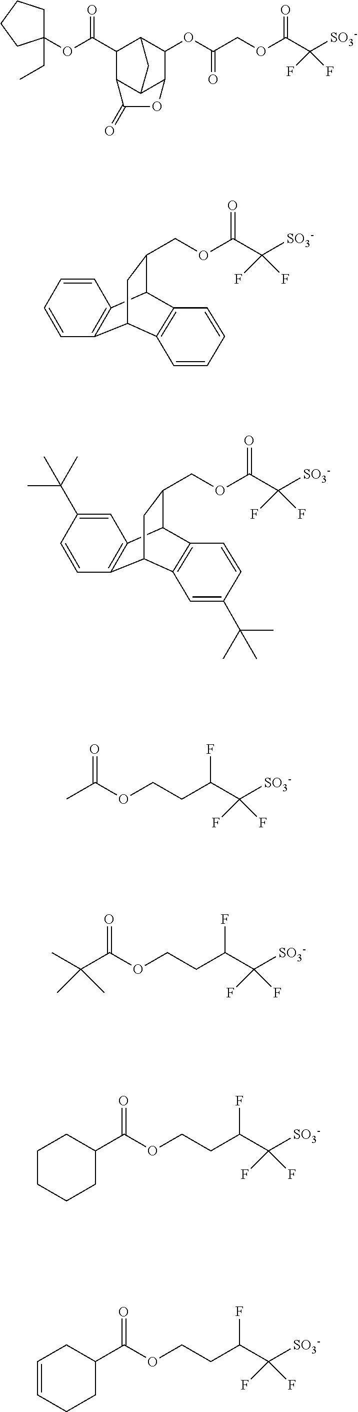

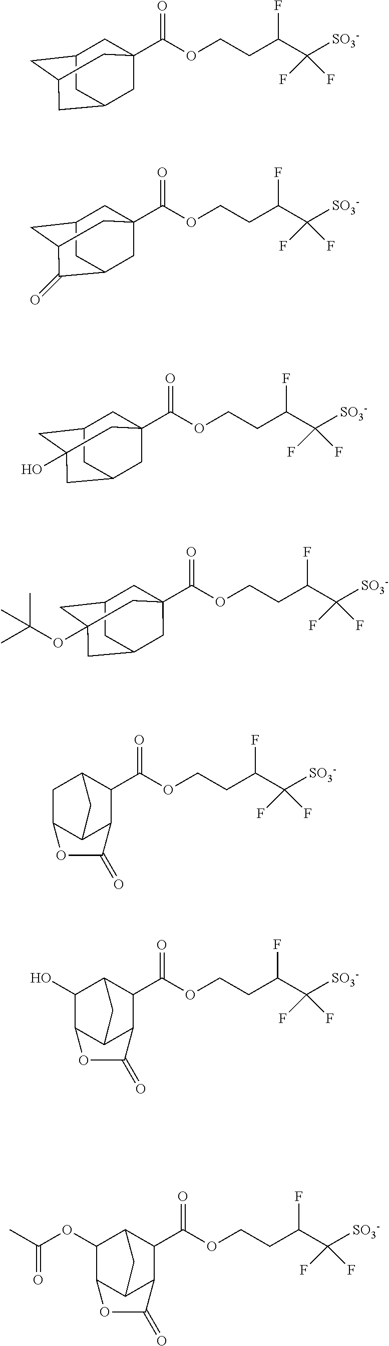

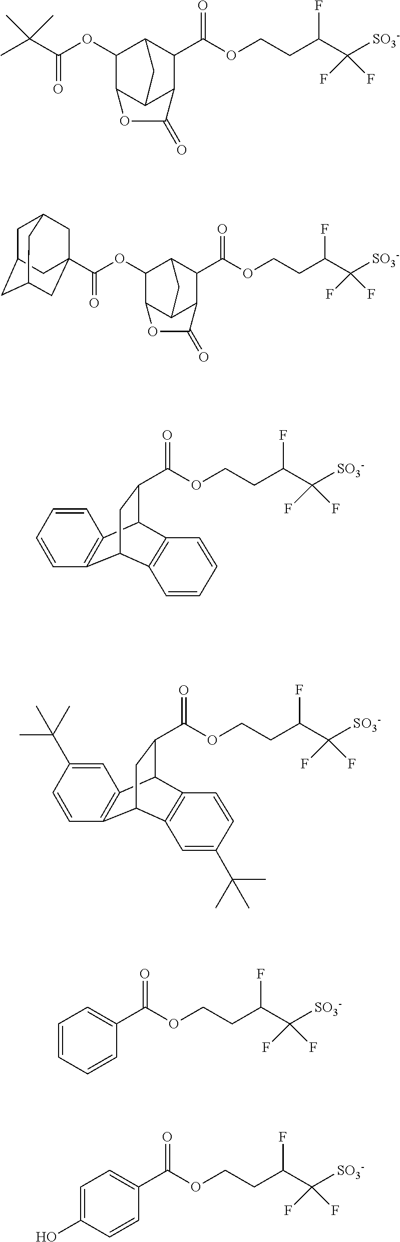

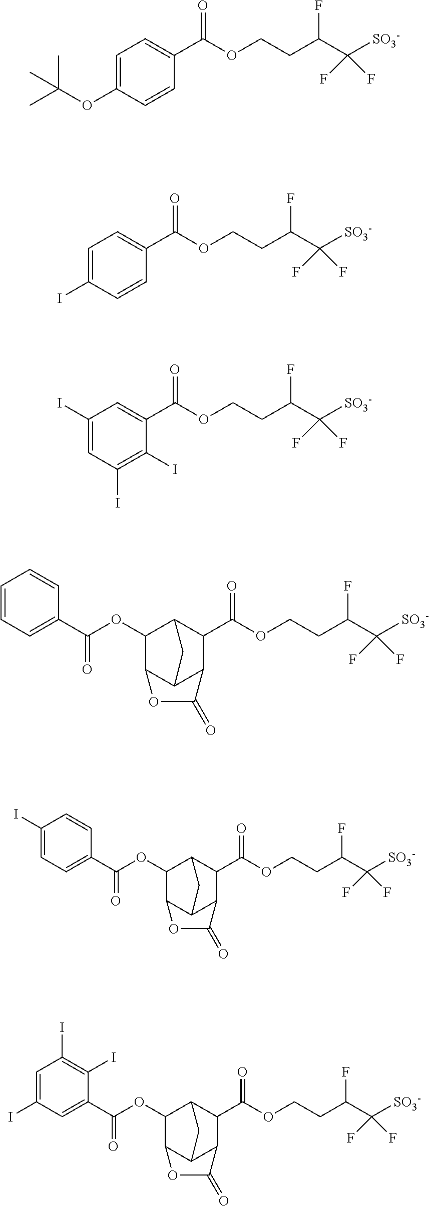

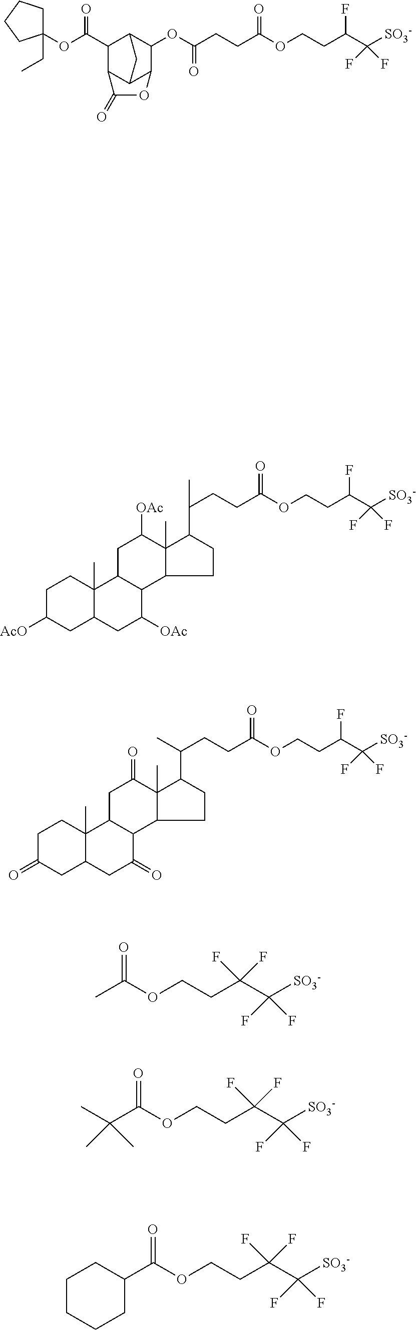

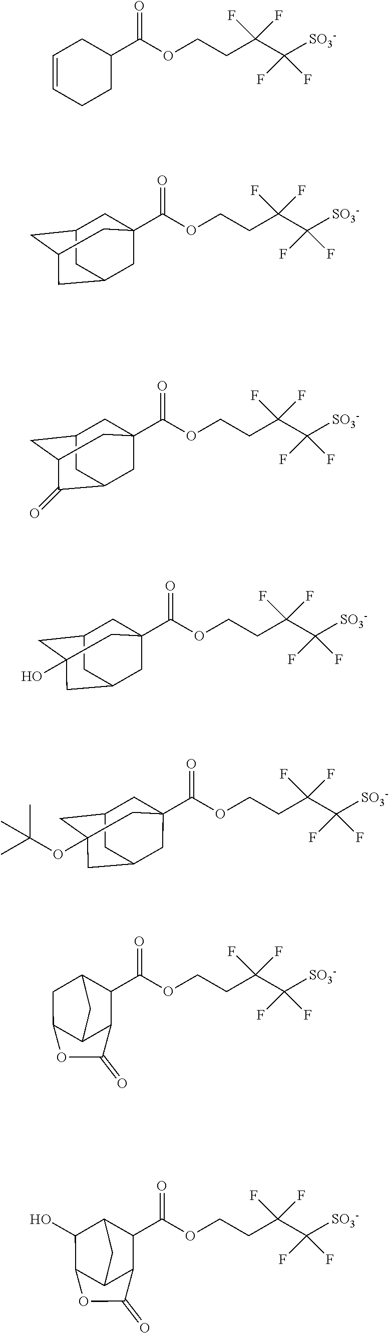

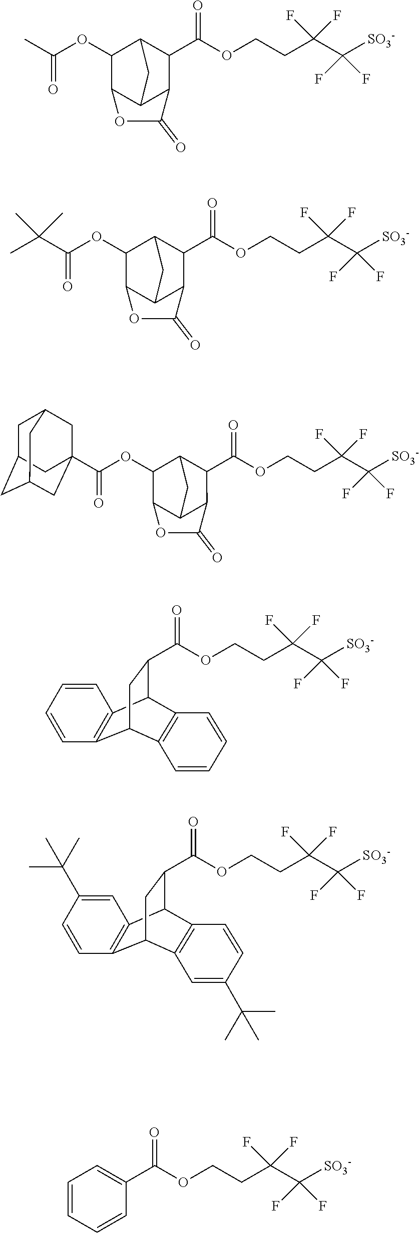

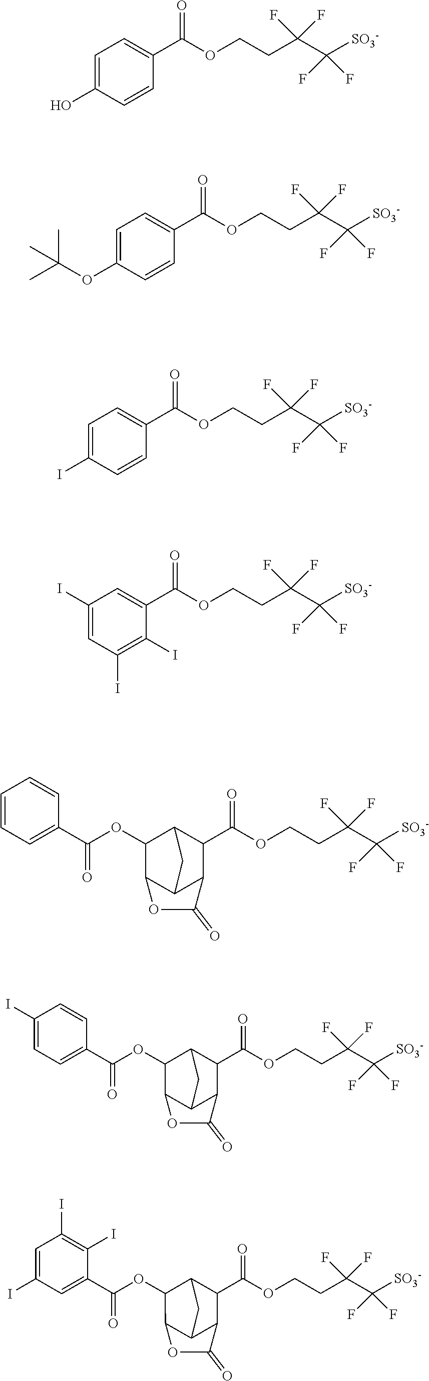

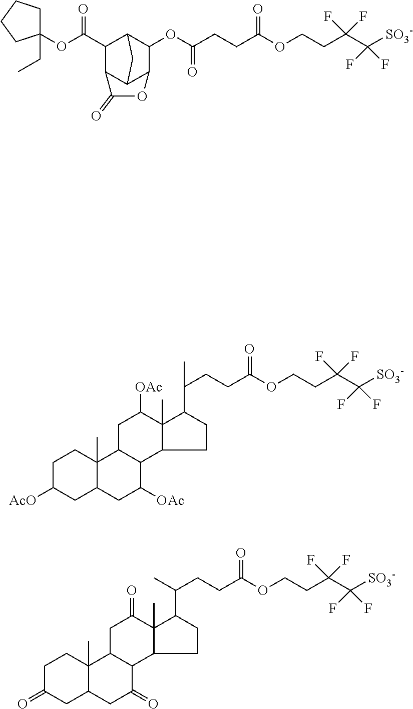

In the formula (Xa-2), R^{fb1} and R^{fb2} are each independently a fluorine atom or a C₁-C₄₀ hydrocarbyl group which may contain a heteroatom. The hydrocarbyl group may be saturated or unsaturated and straight, branched, or cyclic. Specific examples thereof are as exemplified above for the hydrocarbyl group of R^{fa1} in the formula (Xa-1-1). R^{fb1} and R^{fb2} are preferably a fluorine atom or a straight C₁-C₄ fluorinated alkyl group. R^{fb1} and R^{fb2} may bond together to form a ring with a group (—CF₂—SO₂—N⁻—SO₂—CF₂—) to which they are attached, and in this case, the group obtained by bonding R^{fb1} and R^{fb2} is preferably a fluorinated ethylene group or a fluorinated propylene group.

In the formula (Xa-3), R^{fc1}, R^{fc2}, and R^{fc3} are each independently a fluorine atom or a C₁-C₄₀ hydrocarbyl group which may contain a heteroatom. The hydrocarbyl group may be saturated or unsaturated and straight, branched, or cyclic. Specific examples thereof are as exemplified above for the hydrocarbyl group of R^{fa1} in the formula (Xa-1-1). R^{fc1}, R^{fc2}, and R^{fc3} are preferably a fluorine atom or a straight C₁-C₄ fluorinated alkyl group. R^{fc1} and R^{fc2} may bond together to form a ring with a group (—CF₂—SO₂—C—SO₂—CF₂—) to which they are attached, and in this case, the group obtained by bonding R^{fc1} and R^{fc2} is preferably a fluorinated ethylene group or a fluorinated propylene group.

In the formula (Xa-4), R^{fd} is a C₁-C₄₀ hydrocarbyl group which may contain a heteroatom. The hydrocarbyl group may be saturated or unsaturated and straight, branched, or cyclic. Specific examples thereof are as exemplified above for the hydrocarbyl group of R^{fa1} in the formula (Xa-1-1).

Specific examples of the anion having the formula (Xa-4) include those shown below, but are not limited thereto.

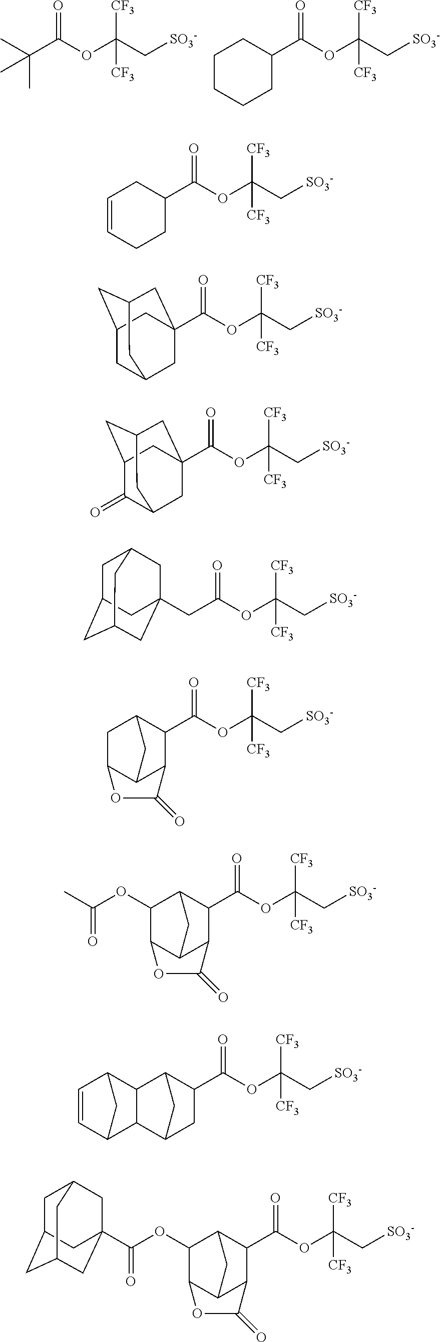

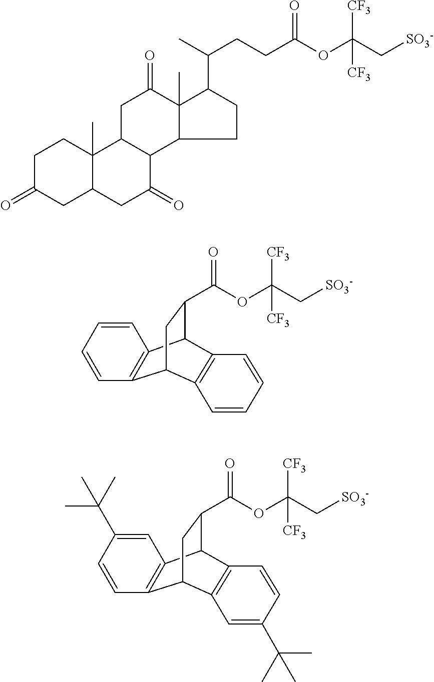

Examples of the non-nucleophilic counterion further include an anion having an aromatic ring substituted with an iodine atom or a bromine atom. Specific examples of the anion include anions having the formula (Xa-5).

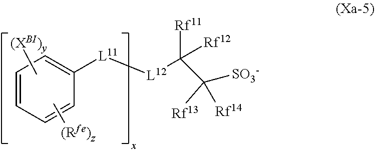

In the formula (Xa-5), x is 1, 2, or 3, y is 1, 2, 3, 4, or 5,
z is 0, 1, 2, or 3, provided that 1≤y+z≤5. y is preferably 1, 2, or 3, and more preferably 2 or 3. z is preferably 0, 1, or 2.

In the formula (Xa-5), X^{BI} is an iodine atom or a bromine atom, and may be identical or different when x and/or y is 2 or more.

In the formula (Xa-5), L¹¹ is a single bond, an ether bond, an ester bond, or a C₁-C₆ saturated hydrocarbylene group which may contain an ether bond or an ester bond. The saturated hydrocarbylene group may be straight, branched, or cyclic.

In the formula (Xa-5), L¹² is a single bond or a C₁-C₂₀ divalent linking group when x is 1, and a C₁-C₂₀ (x+1)-valent linking group when x is 2 or 3, and the linking group may contain an oxygen atom, a sulfur atom, or a nitrogen atom.

In the formula (Xa-5), R^{fe} is a hydroxy group, a carboxy group, a fluorine atom, a chlorine atom, a bromine atom, or an amino group, or a C₁-C₂₀ hydrocarbyl group, a C₁-C₂₀ hydrocarbyloxy group, a C₂-C₂₀ hydrocarbylcarbonyl group, a C₂-C₂₀ hydrocarbyloxycarbonyl group, a C₂-C₂₀ hydrocarbylcarbonyloxy group, or a C₁-C₂₀ hydrocarbylsulfonyloxy group, which may contain a fluorine atom, a chlorine atom, a bromine atom, a hydroxy group, an amino group, or an ether bond, or —N(R^{feA})(R^{feB}), —N(R^{feC})—C(═O)—R^{feD}, or —N(R^{feC})—C(═O)—O—R^{feD}. R^{feA} and R^{feB} are each independently a hydrogen atom or a C₁-C₆ saturated hydrocarbyl group, R^{feC} is a hydrogen atom or a C₁-C₆ saturated hydrocarbyl group, and may contain a halogen atom, a hydroxy group, a C₁-C₆ saturated hydrocarbyloxy group, a C₂-C₆ saturated hydrocarbylcarbonyl group, or a C₂-C₆ saturated hydrocarbylcarbonyloxy group. R^{feD} is a C₁-C₁₆ aliphatic hydrocarbyl group, a C₆-C₁₂ aryl group, or a C₇-C₁₅ aralkyl group, and may contain a halogen atom, a hydroxy group, a C₁-C₆ saturated hydrocarbyloxy group, a C₂-C₆ saturated hydrocarbylcarbonyl group, or a C₂-C₆ saturated hydrocarbylcarbonyloxy group. The aliphatic hydrocarbyl group may be saturated or unsaturated and straight, branched, or cyclic. The hydrocarbyl group, hydrocarbyloxy group, hydrocarbylcarbonyl group, hydrocarbyloxycarbonyl group, hydrocarbylcarbonyloxy group, and hydrocarbylsulfonyloxy group may be straight, branched, or cyclic. When x and/or z is 2 or more, a plurality of R^{fe} may be identical or different.

Among them, R^{fe} is preferably a hydroxy group, —N(R^{feC})—C(═O)—R^{feD}, —N(R^{feC})—C(═O)—O—R a fluorine atom, a chlorine atom, a bromine atom, a methyl group, a methoxy group, or the like.

In the formula (Xa-5), Rf¹¹ to Rf¹⁴ are each independently a hydrogen atom, a fluorine atom, or a trifluoromethyl group, and at least one of them is a fluorine atom or a trifluoromethyl group. Rf¹¹ and Rf¹² may be combined to form a carbonyl group. In particular, both Rf¹³ and Rf⁴ are preferably fluorine atoms.

Specific examples of the anion having the formula (Xa-5) include those shown below, but are not limited thereto. In the following formulae, X^{BI} is as defined above.

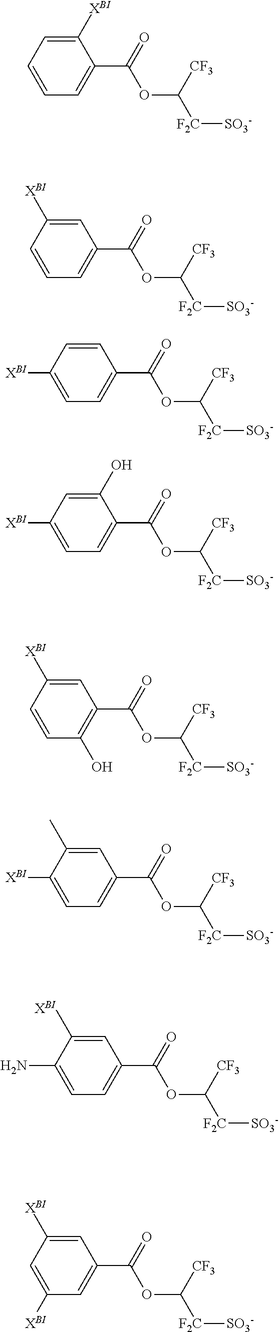

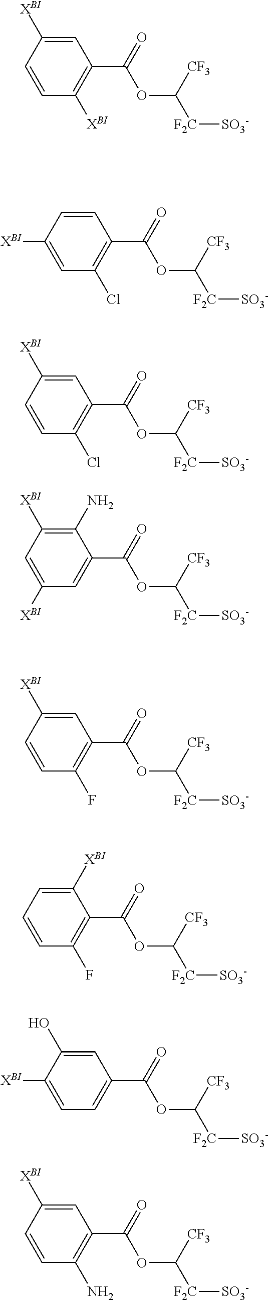

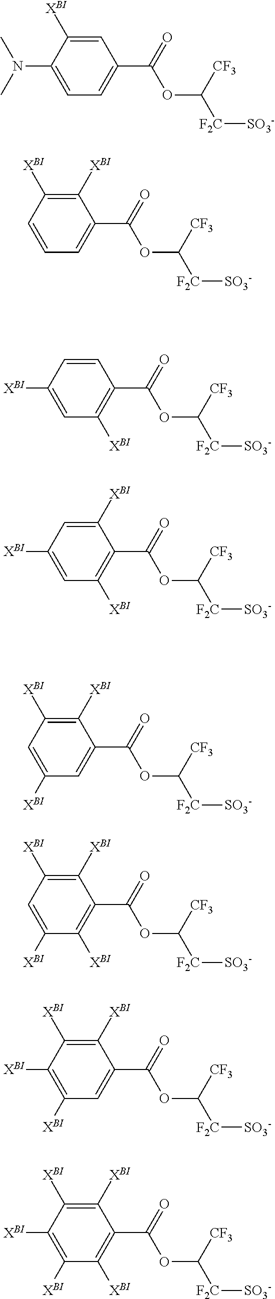

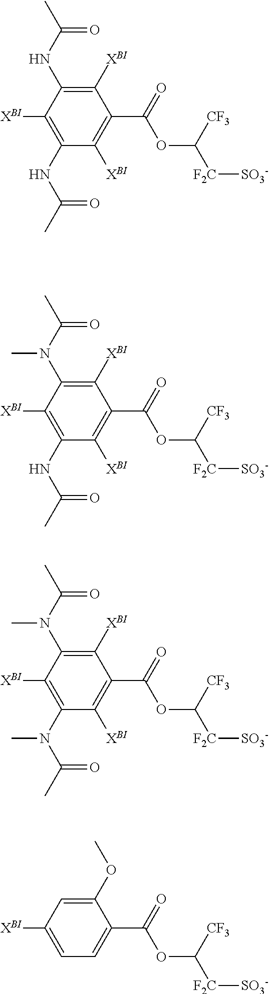

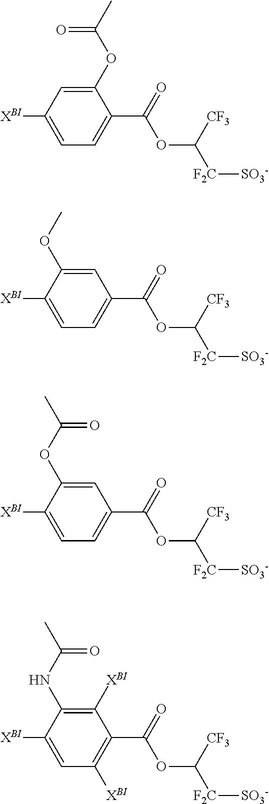

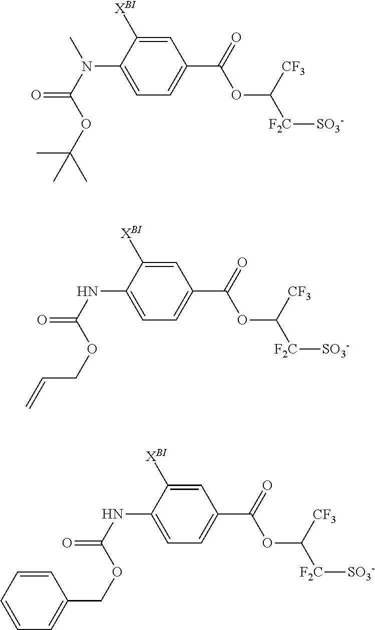

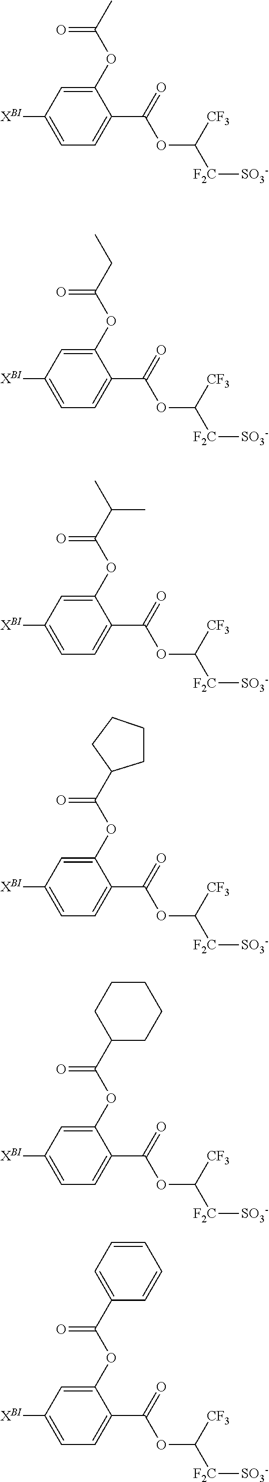

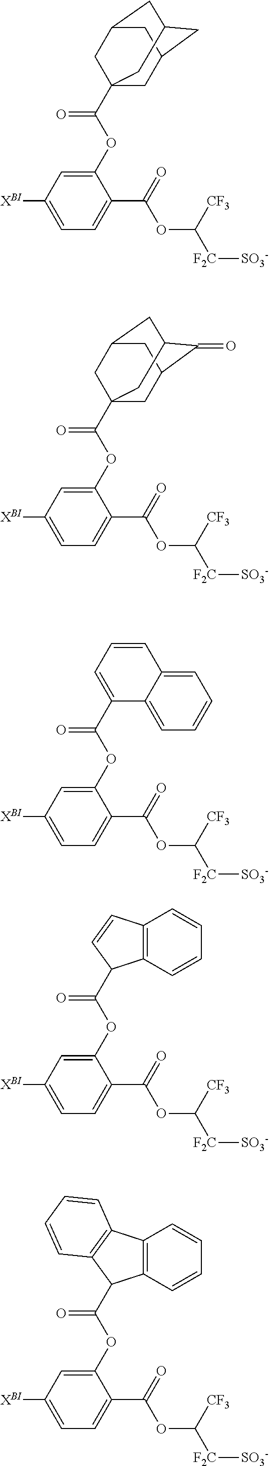

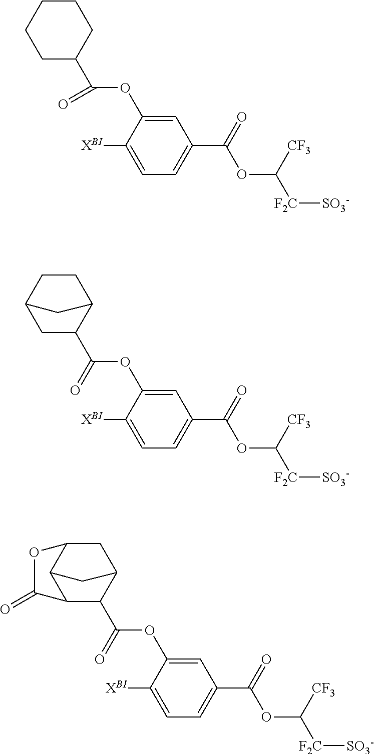

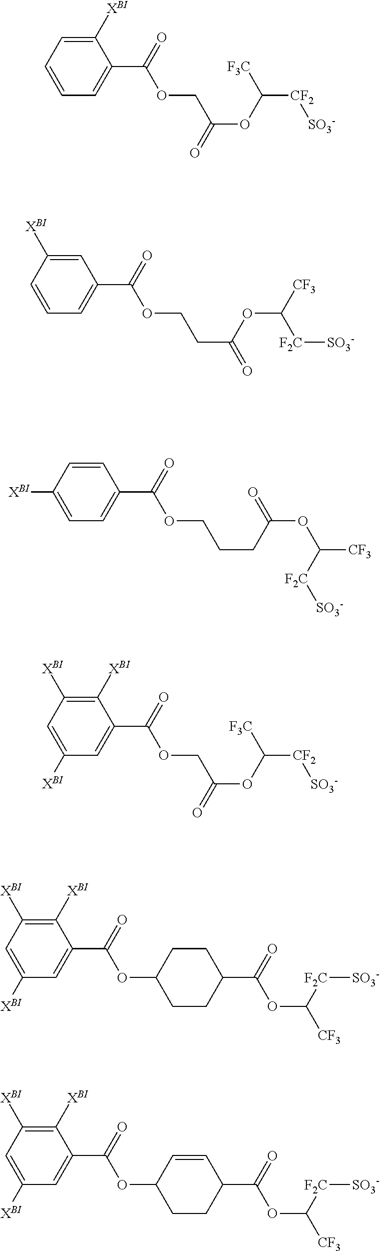

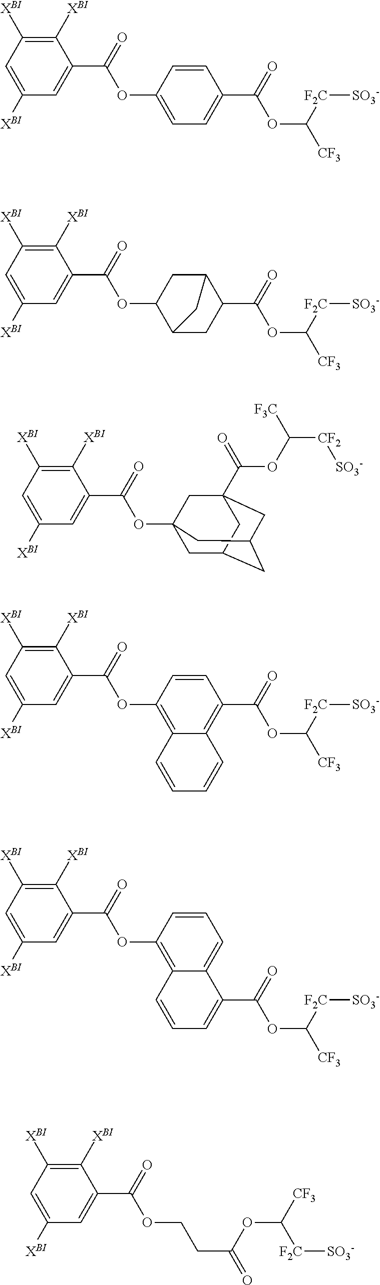

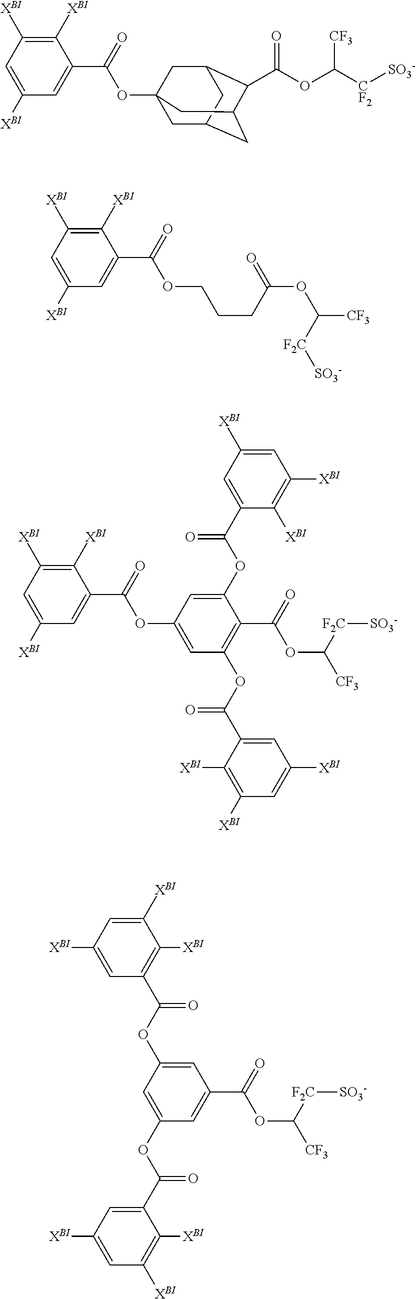

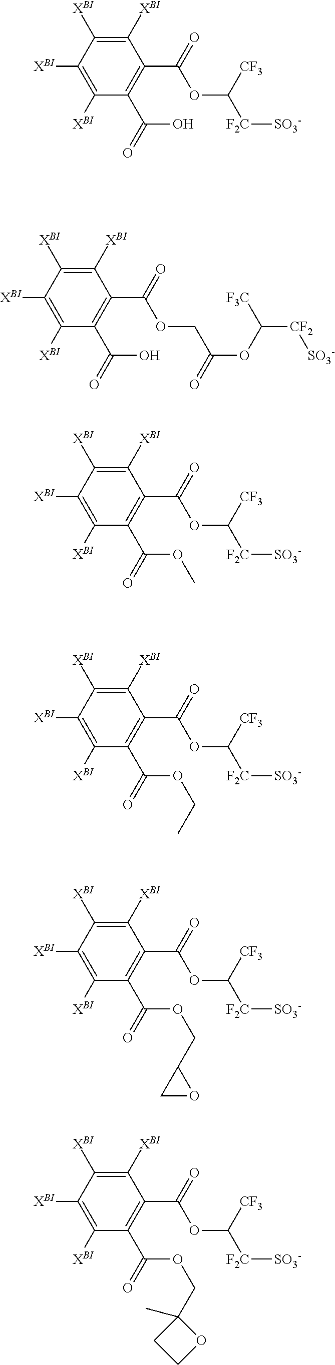

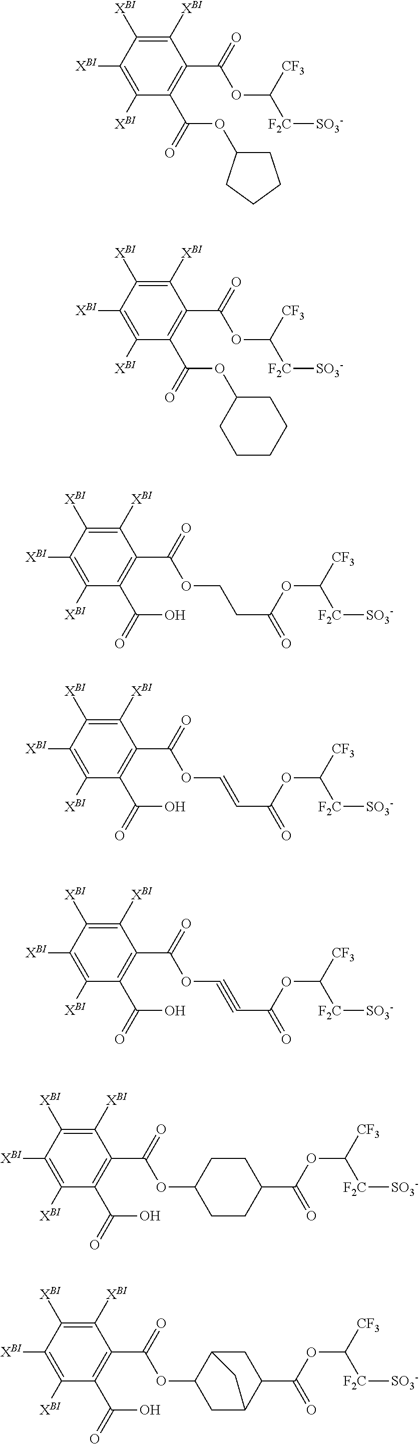

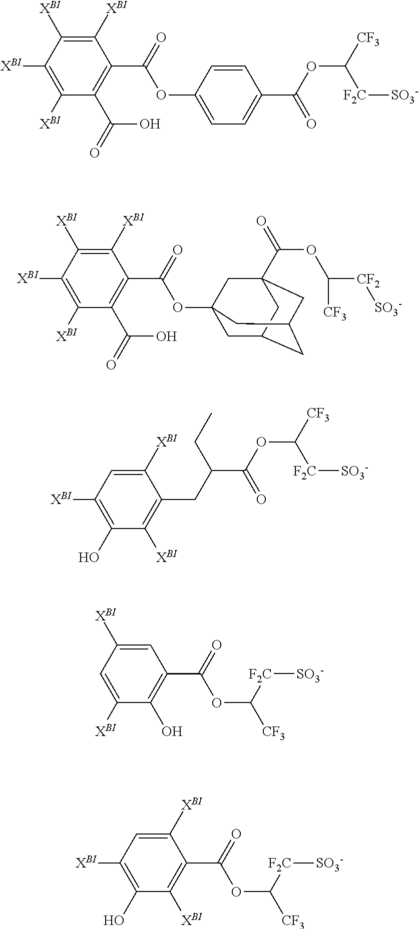

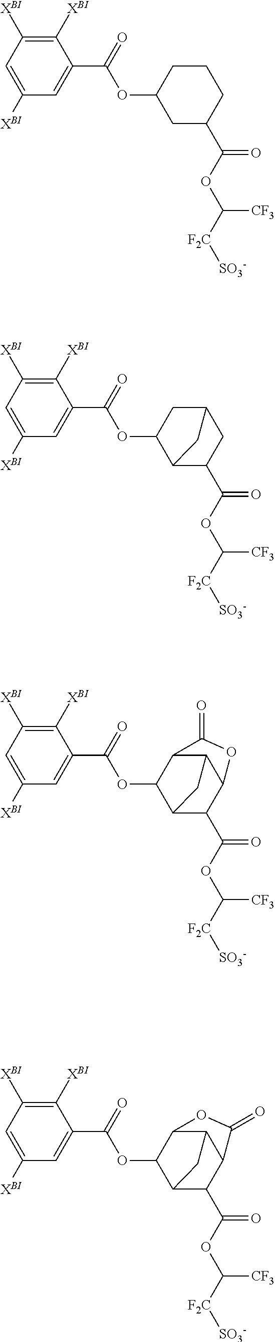

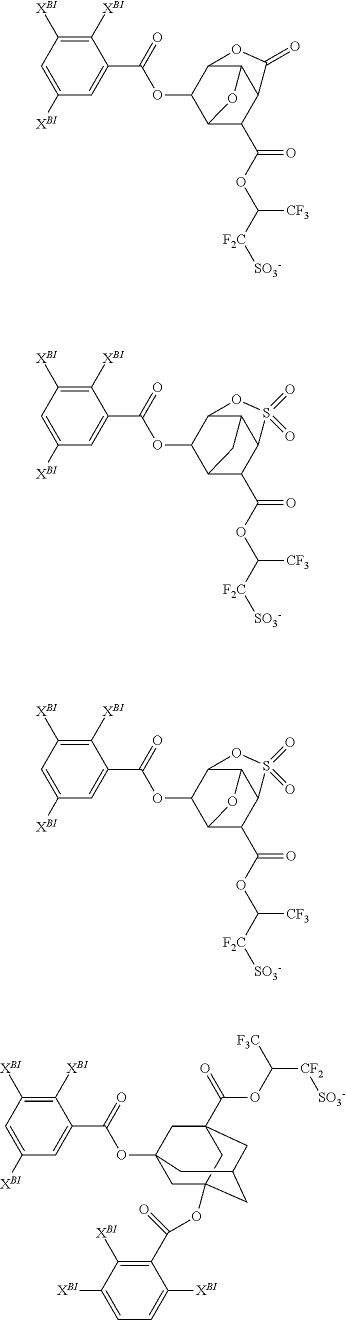

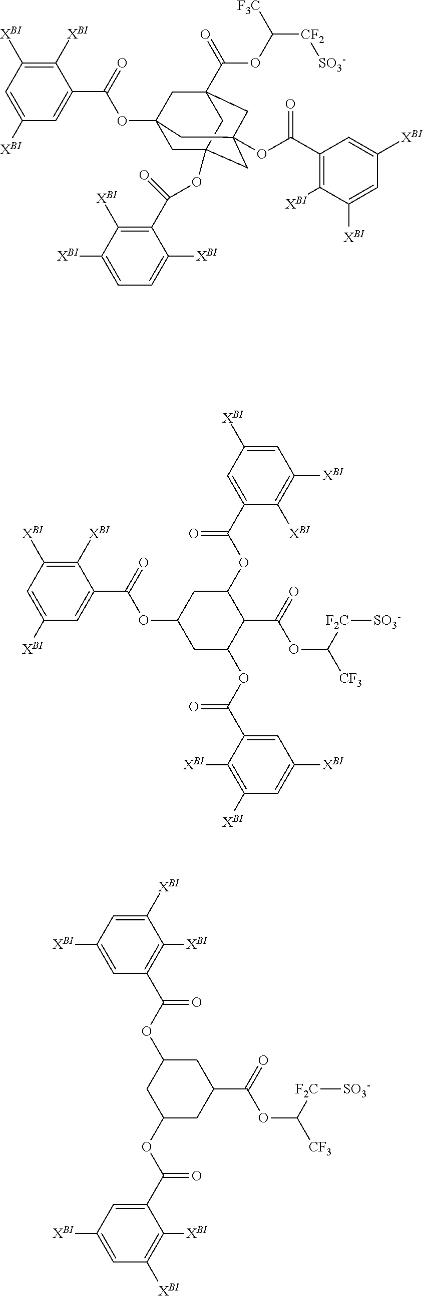

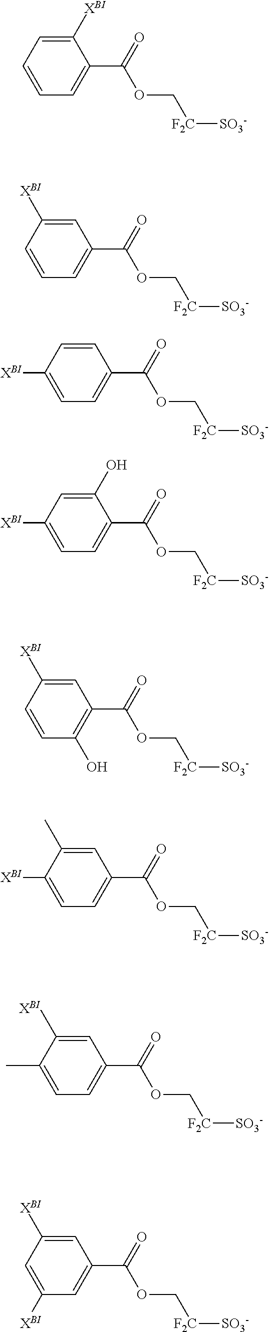

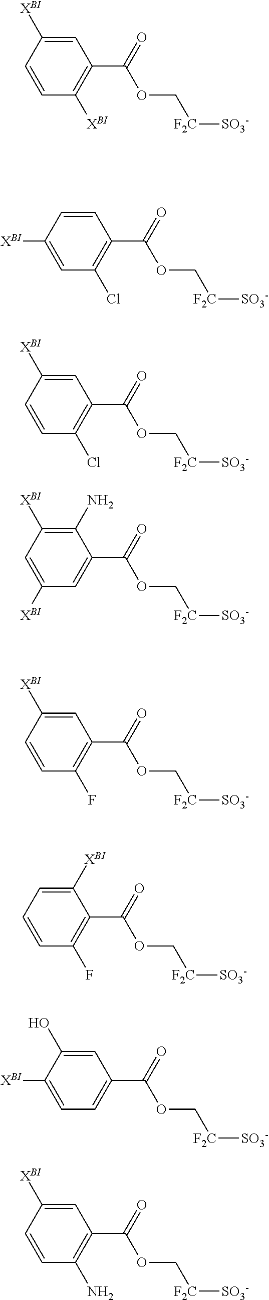

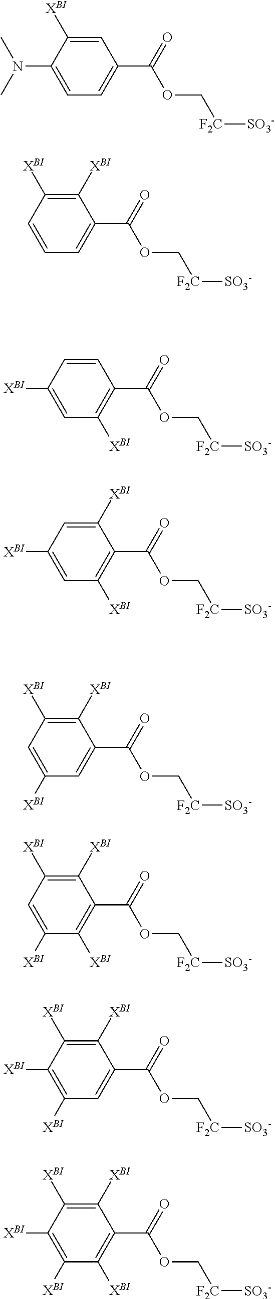

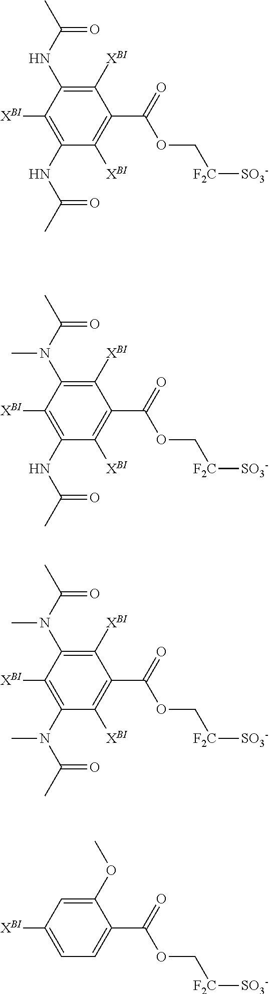

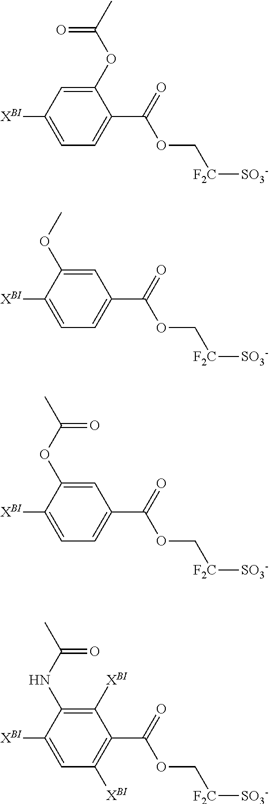

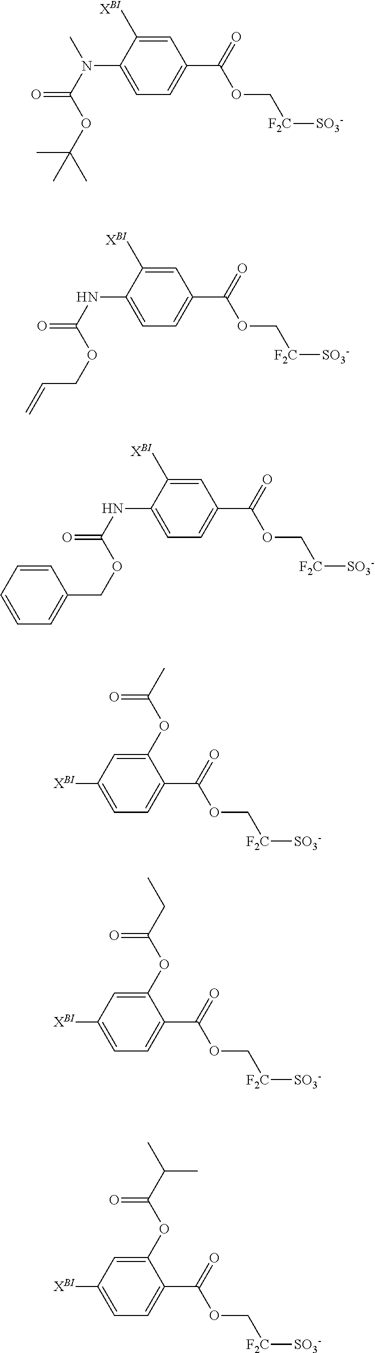

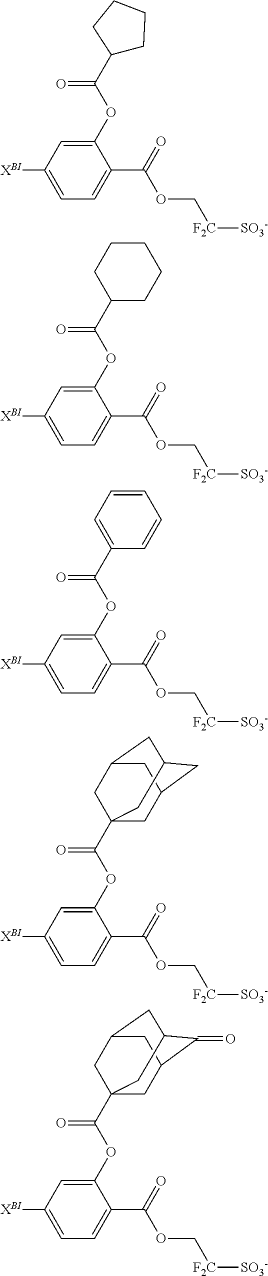

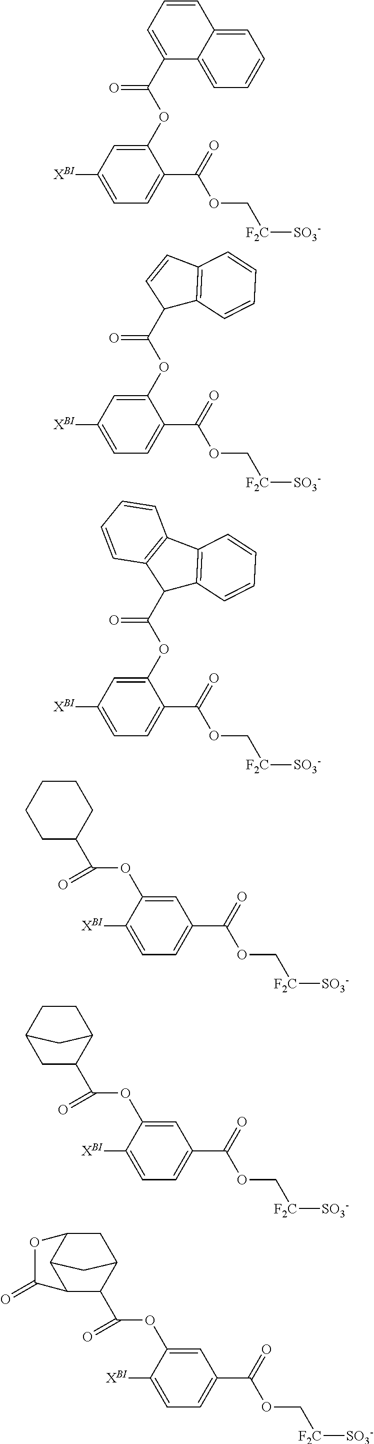

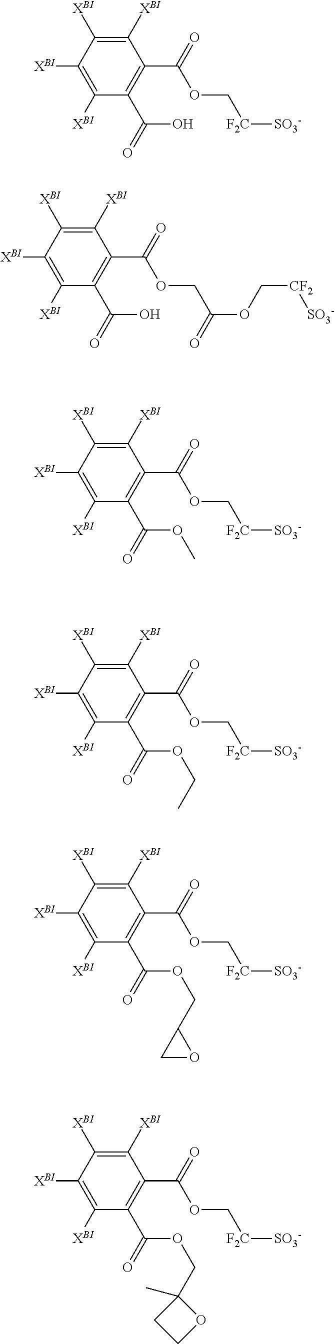

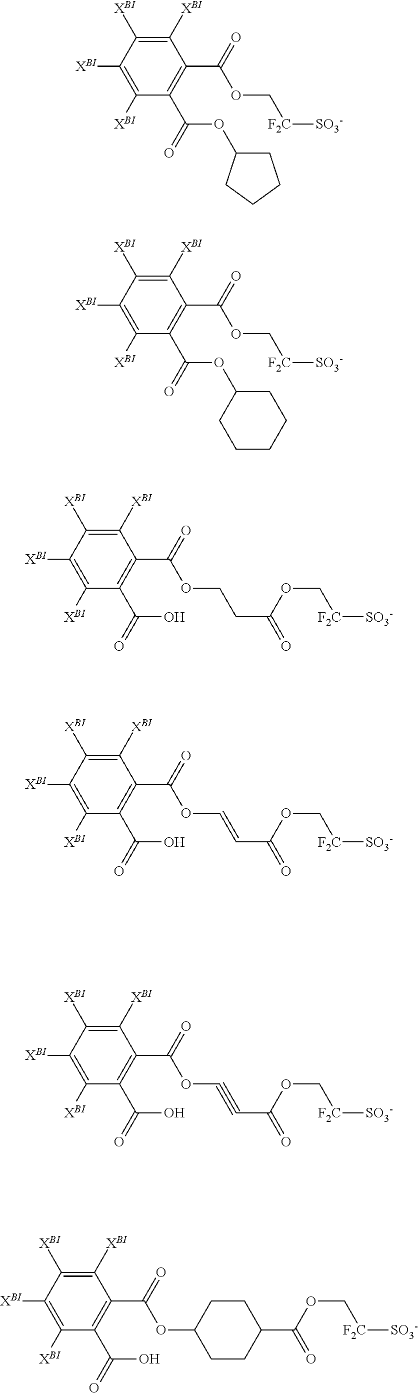

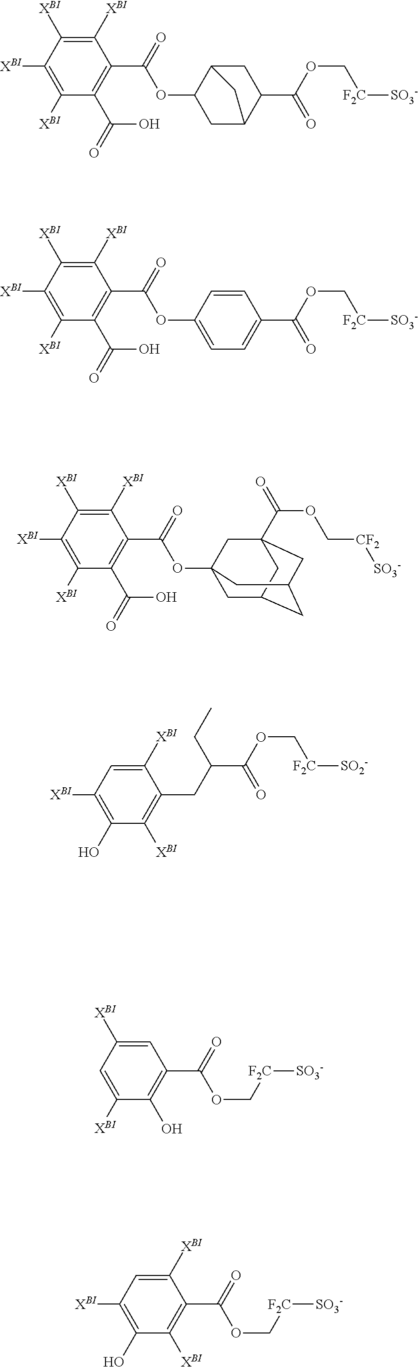

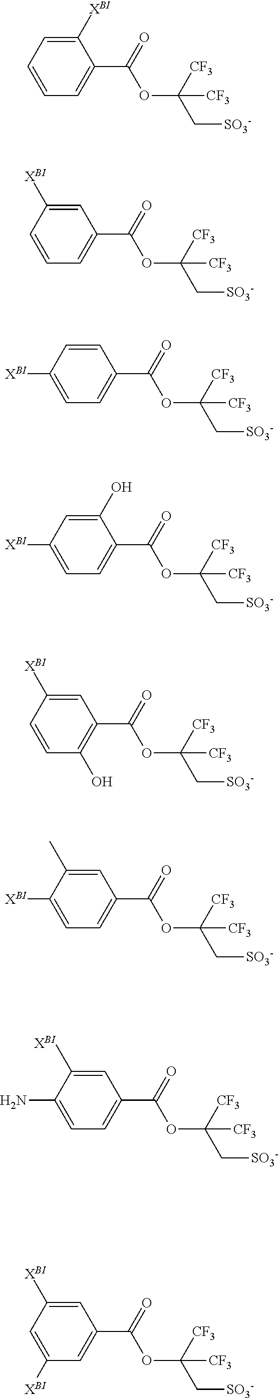

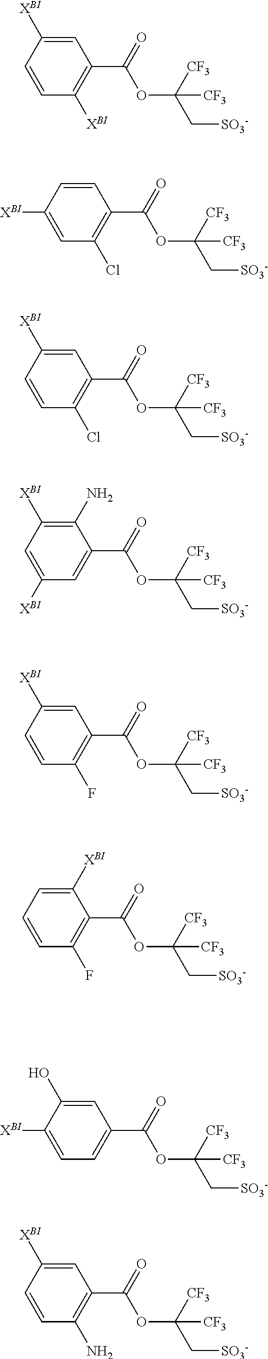

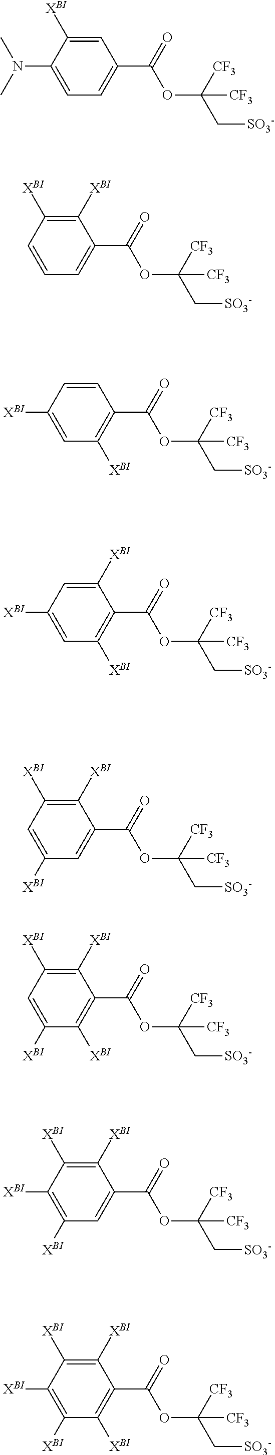

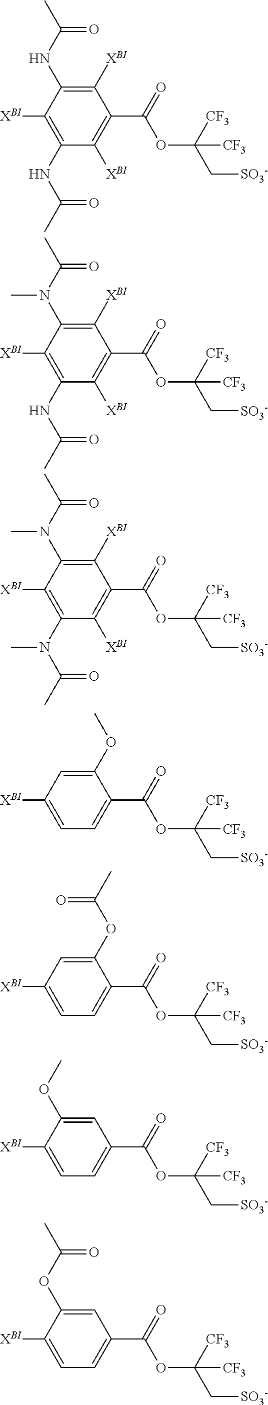

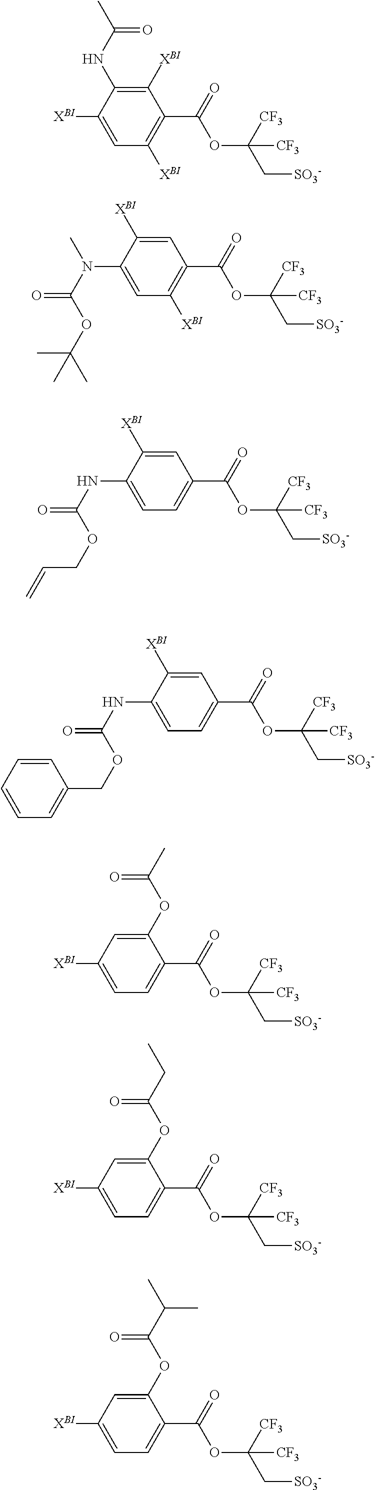

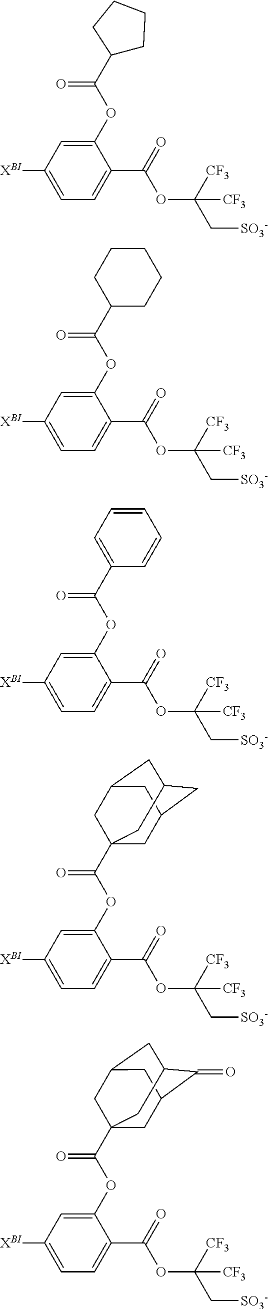

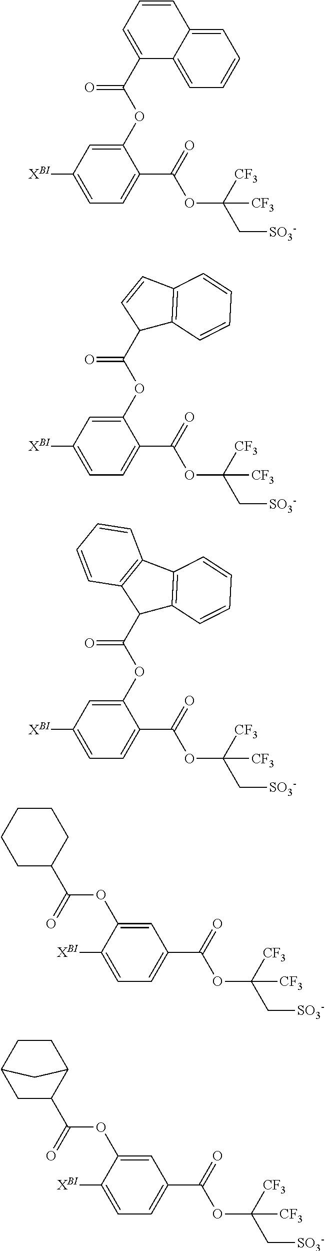

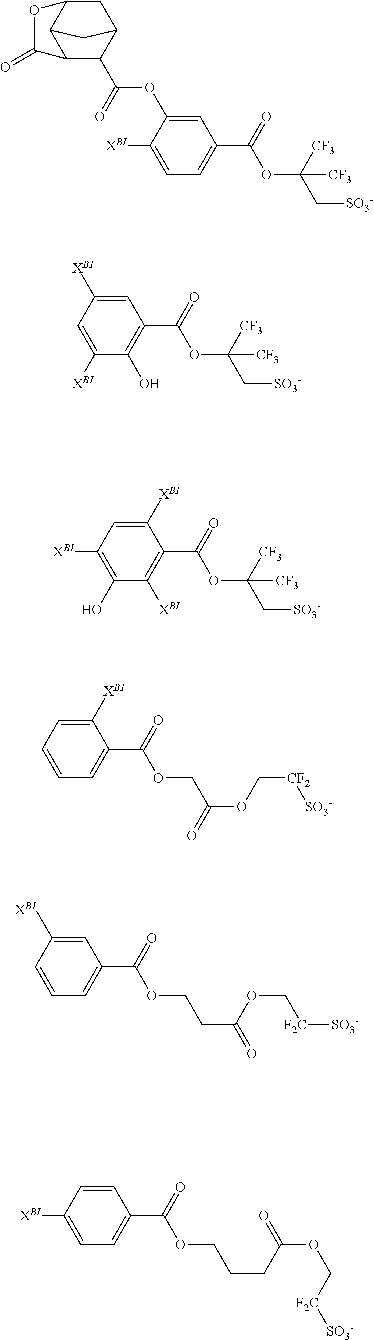

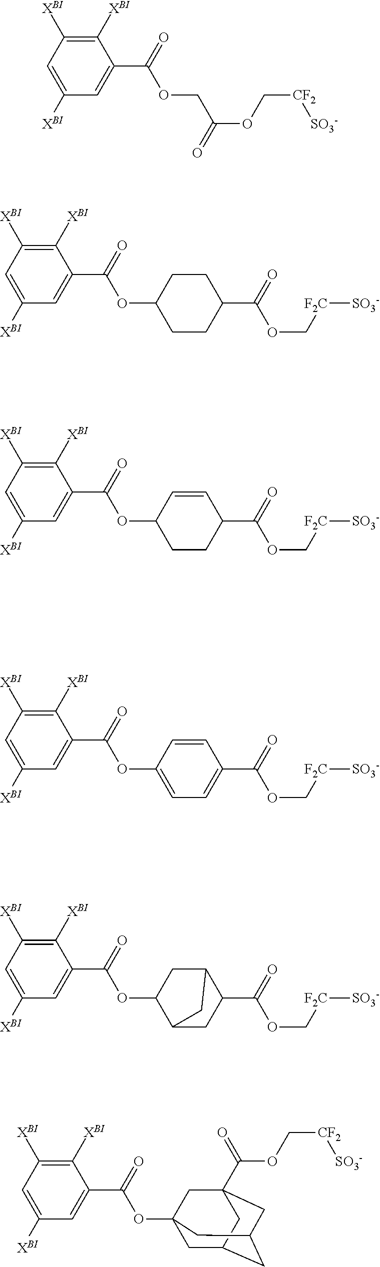

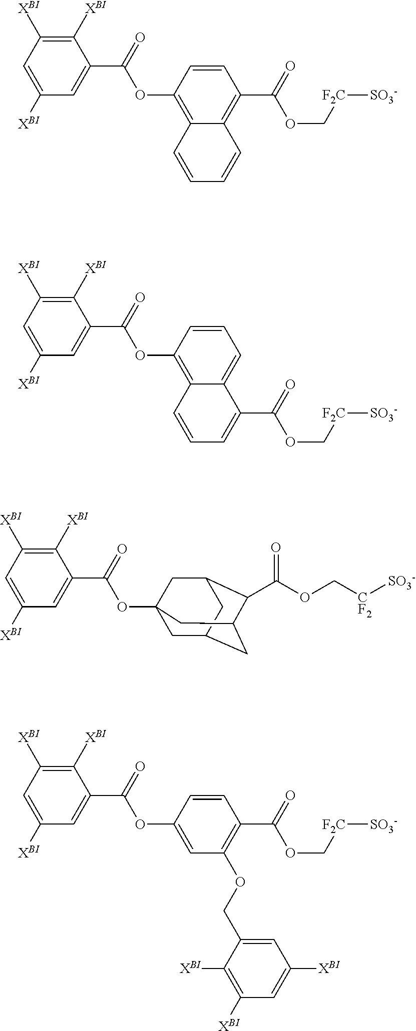

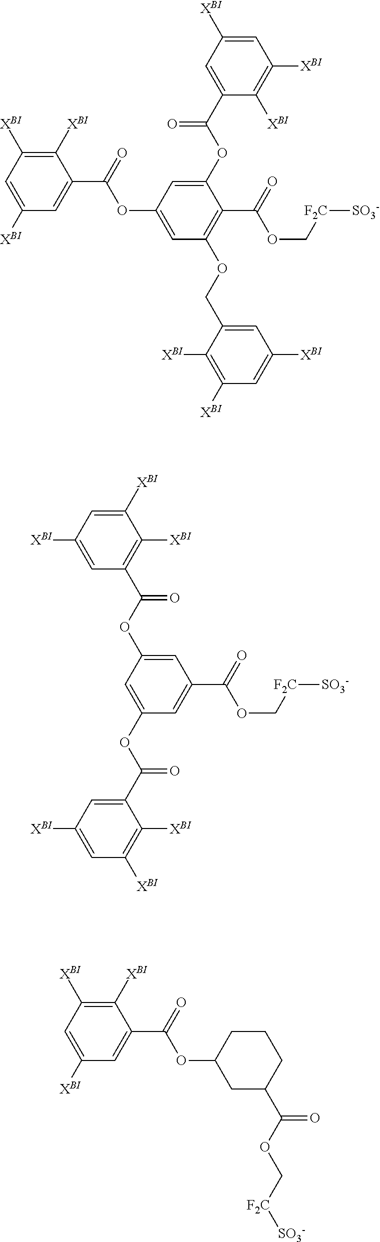

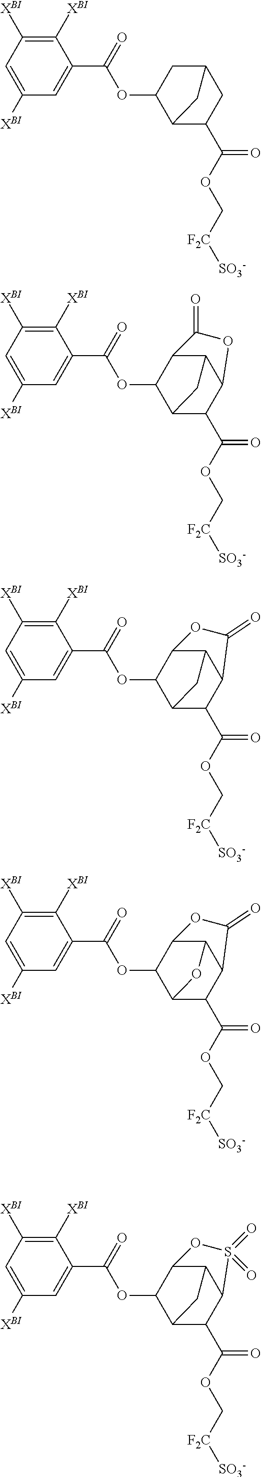

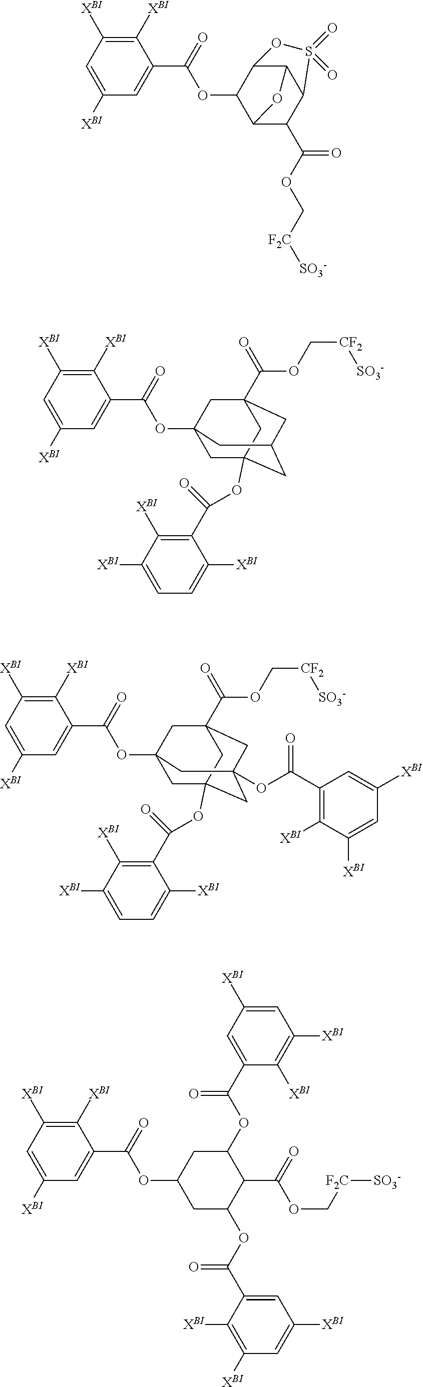

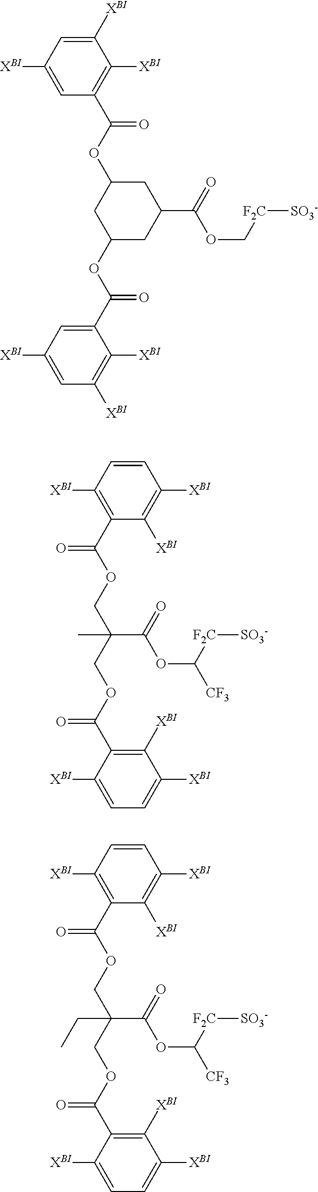

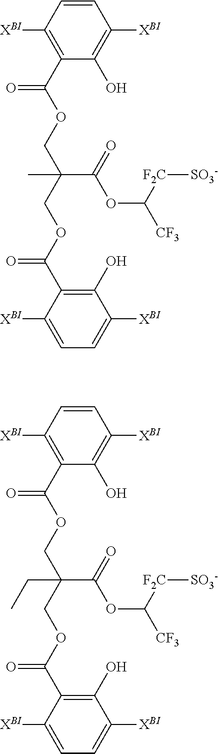

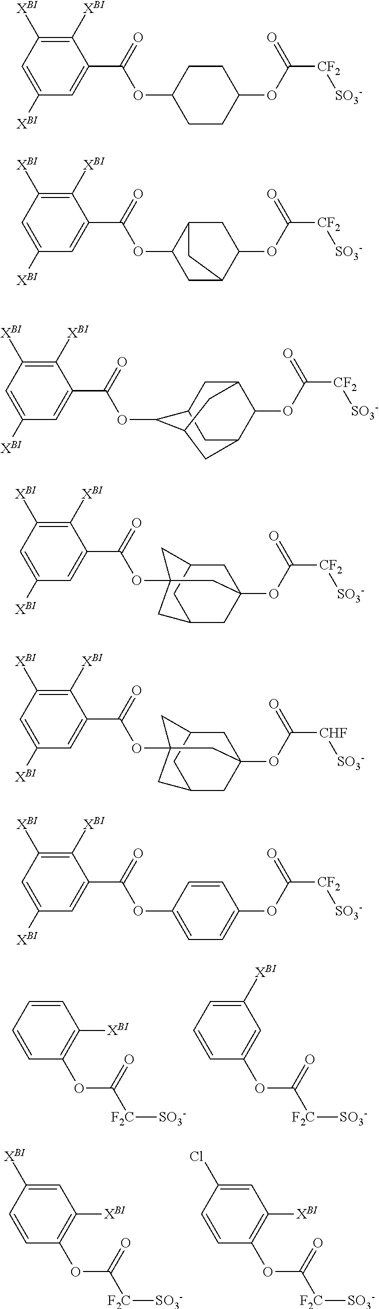

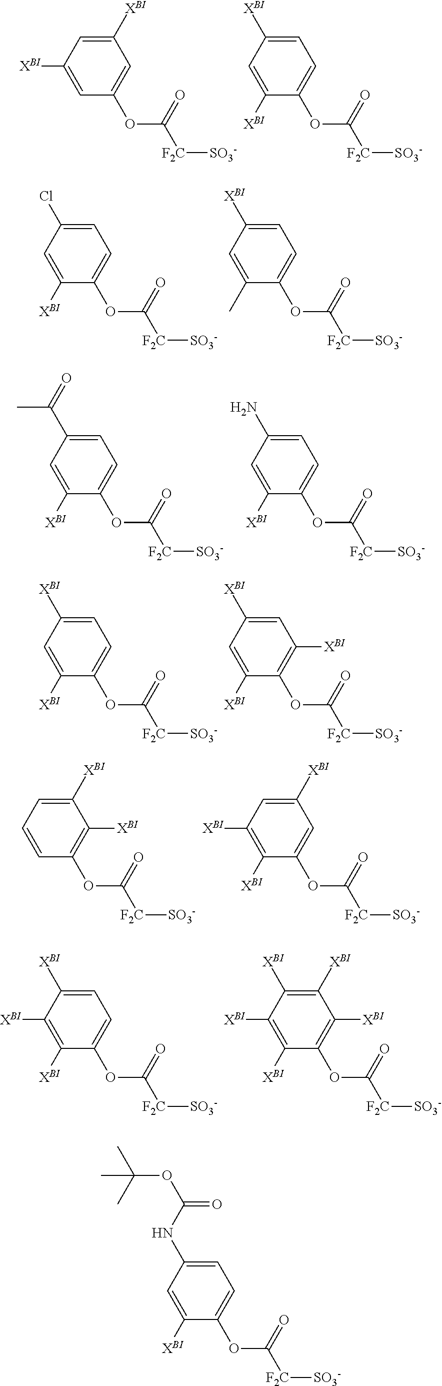

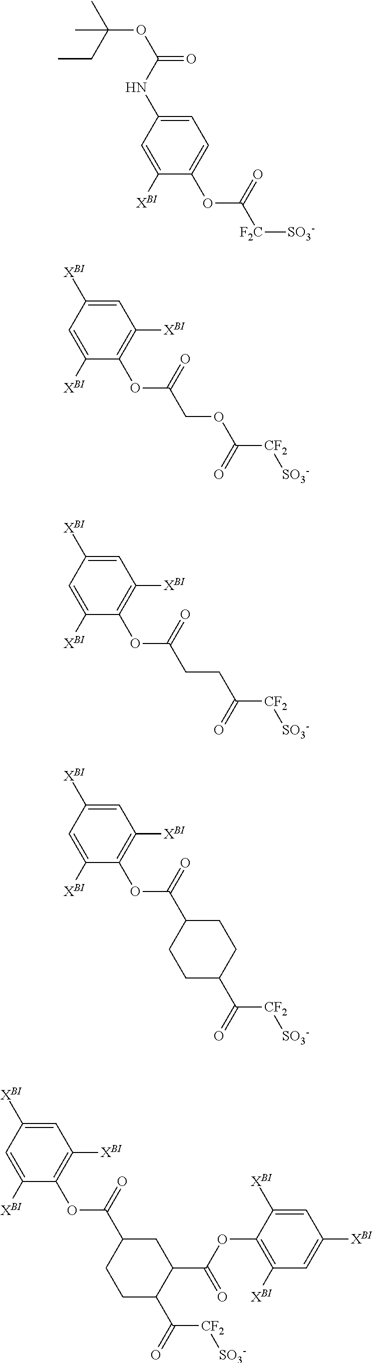

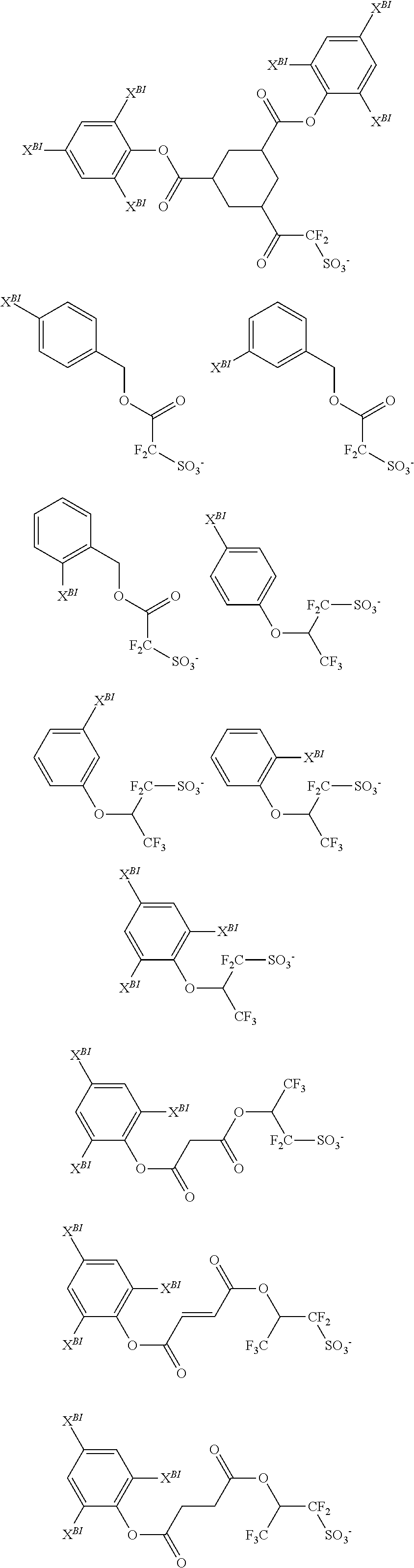

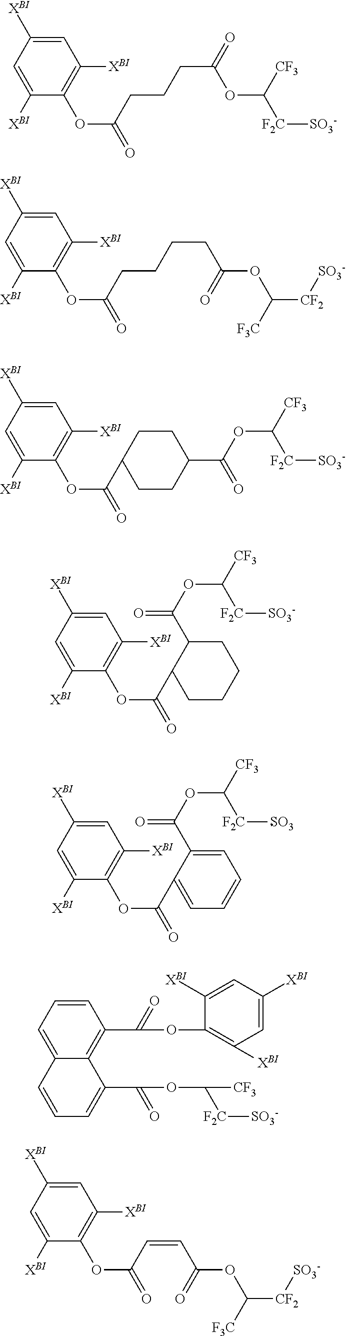

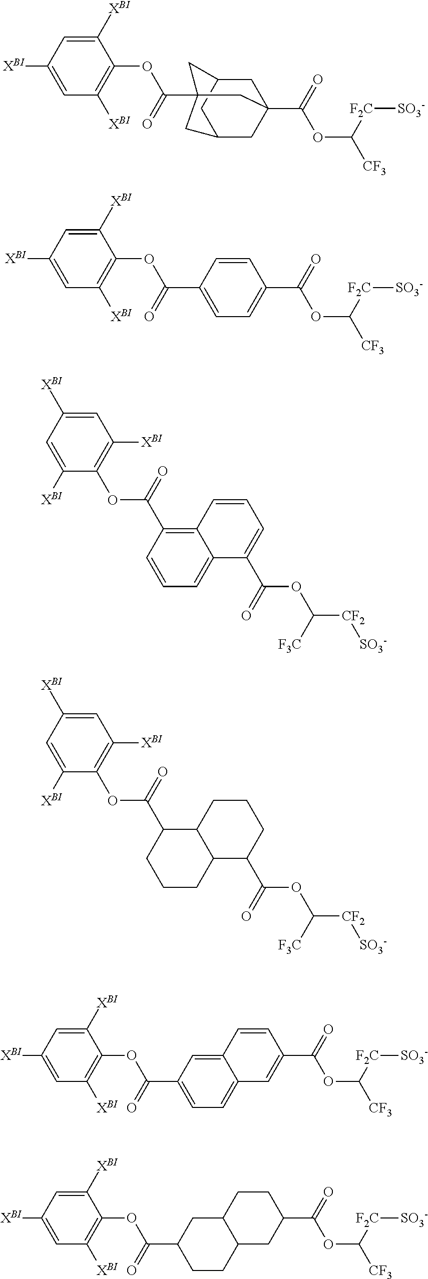

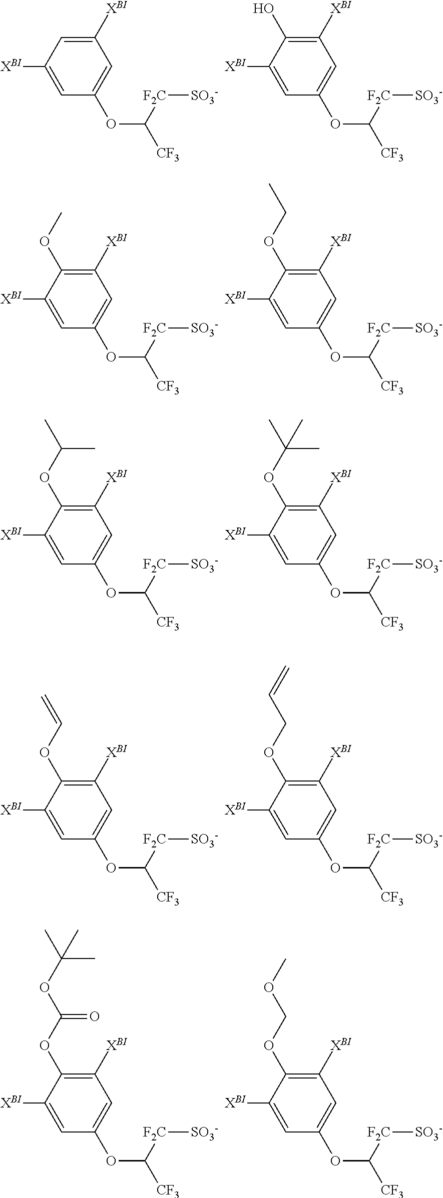

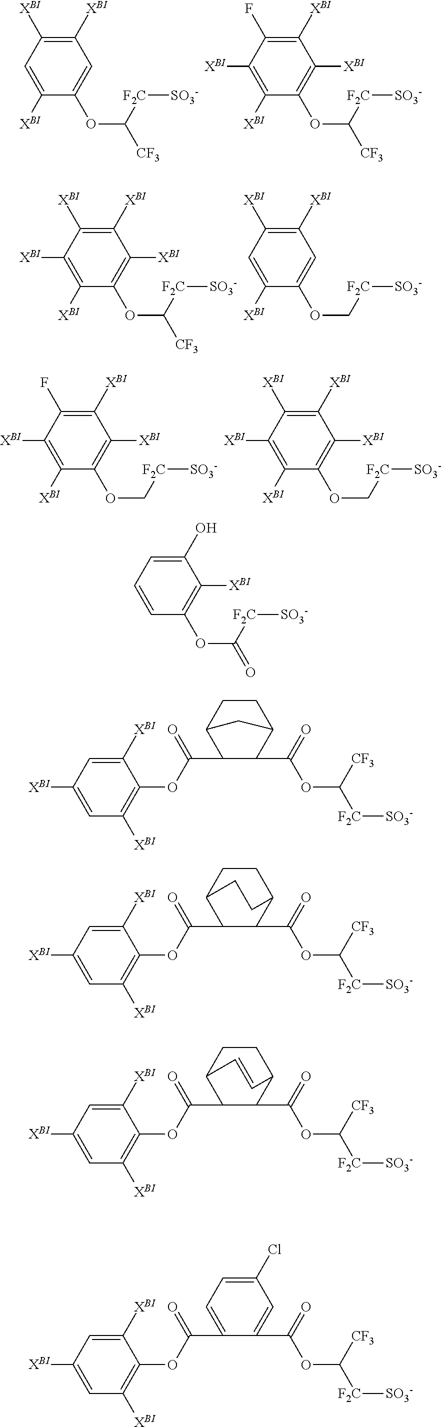

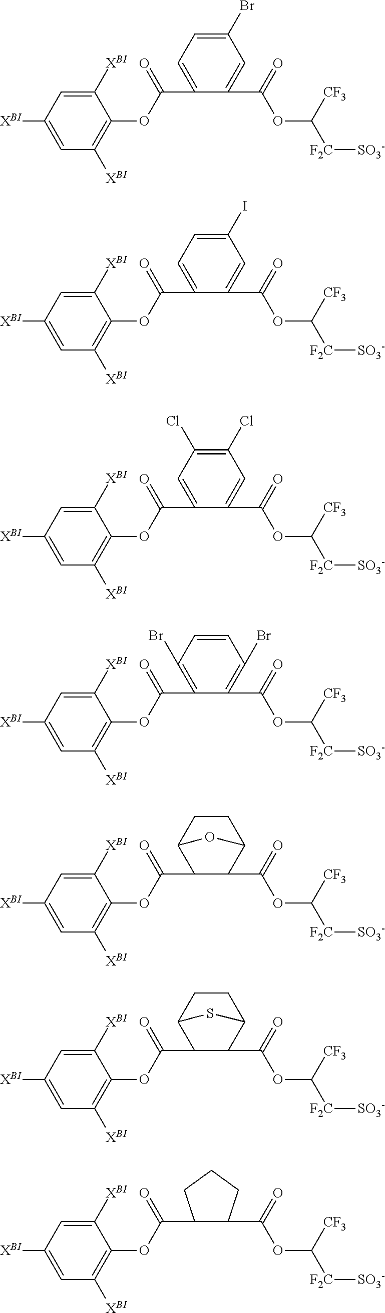

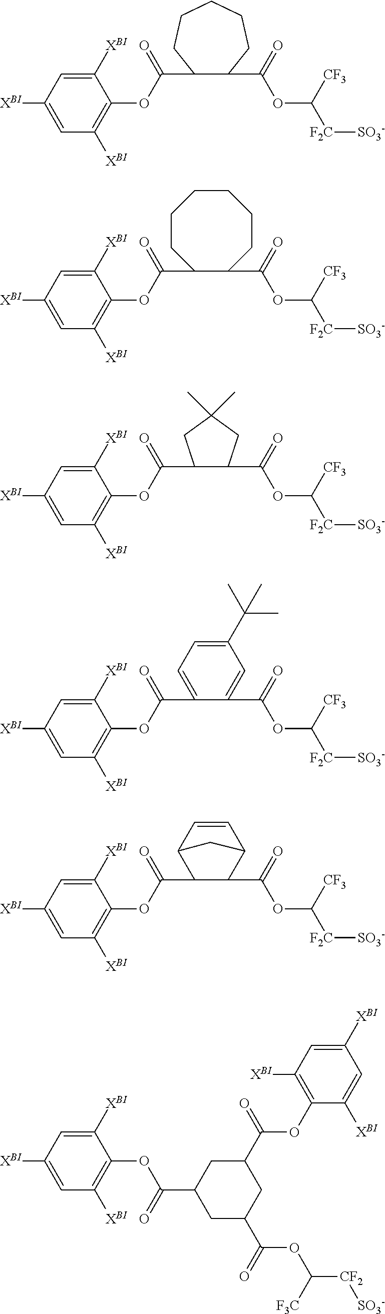

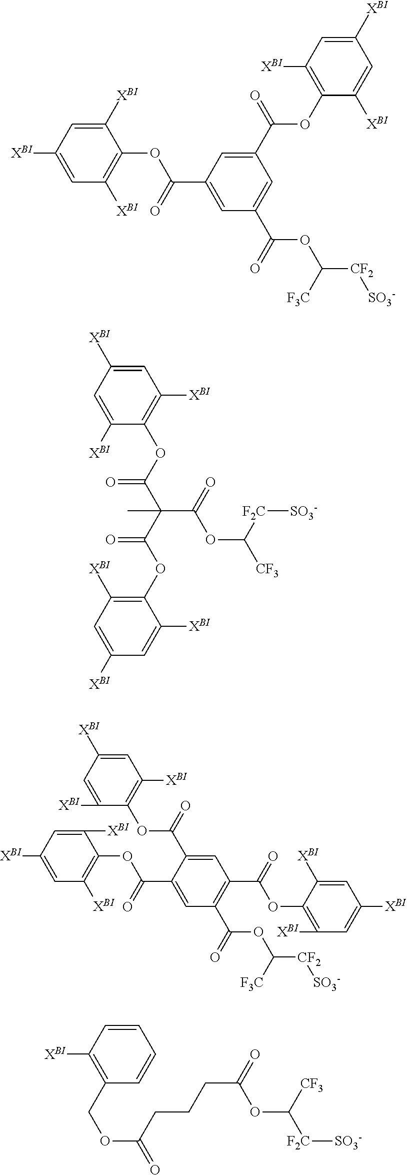

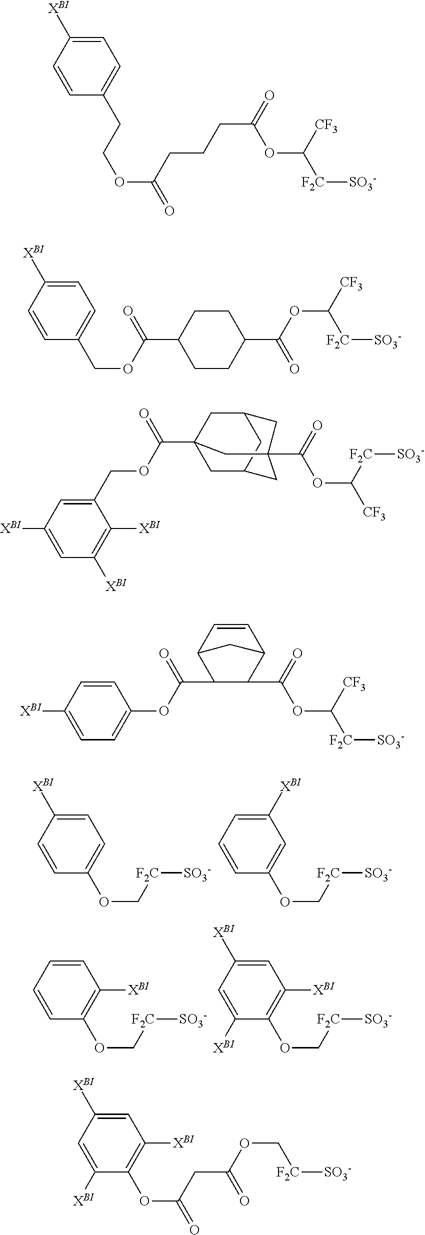

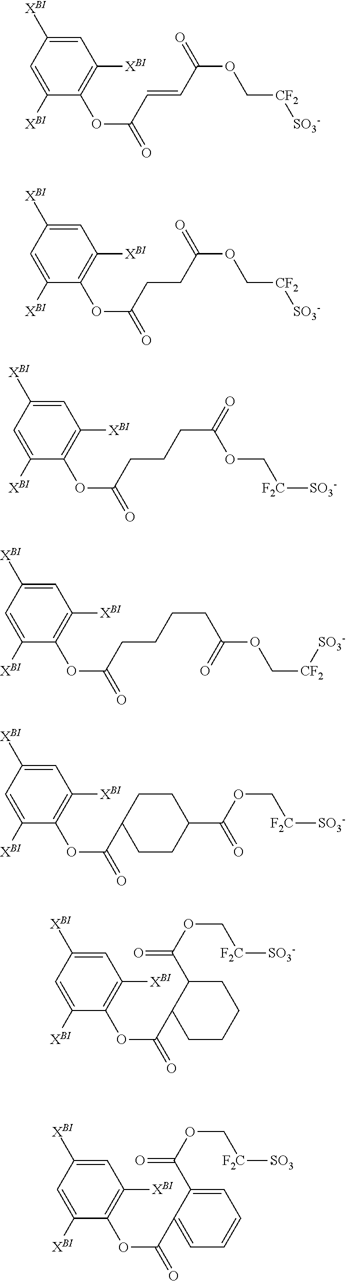

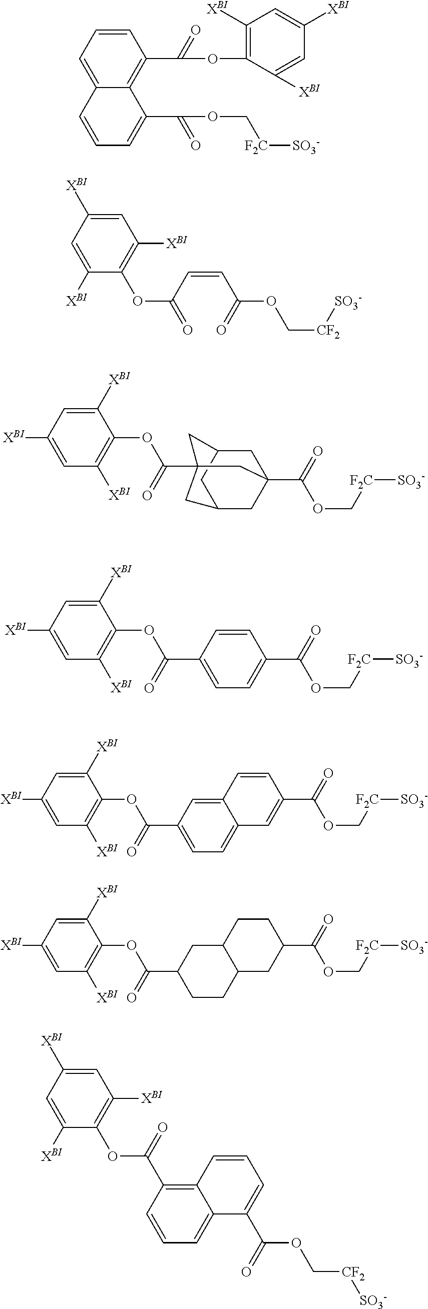

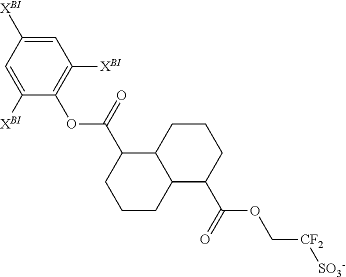

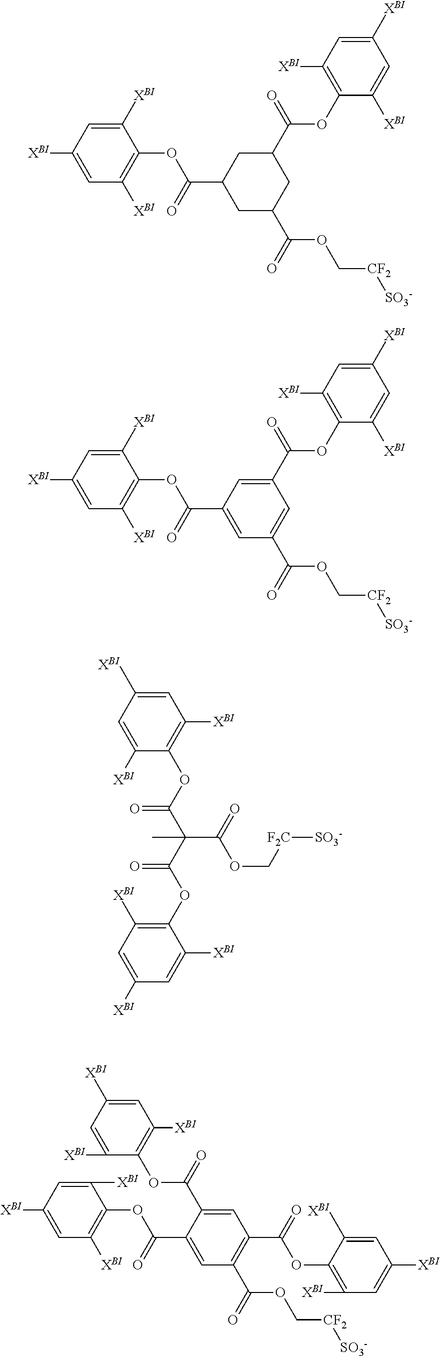

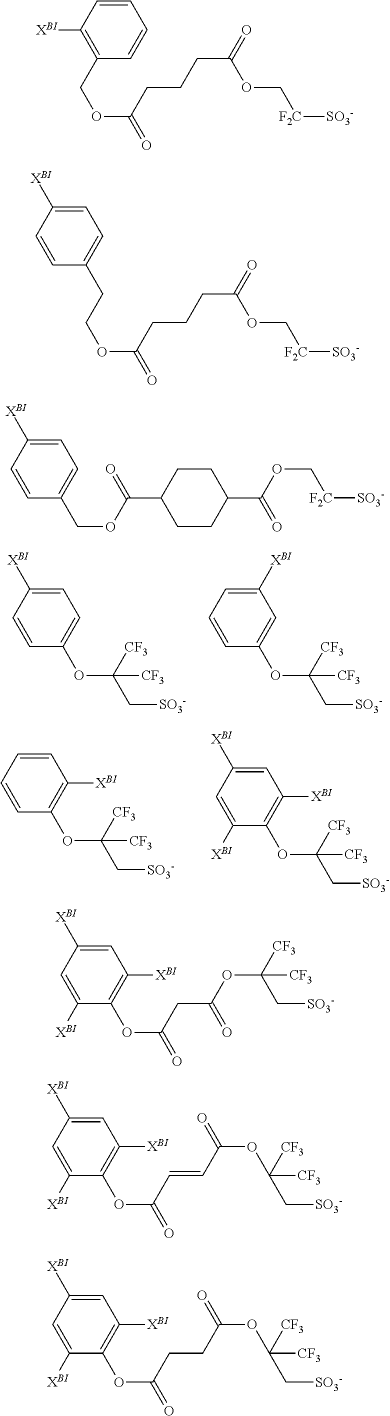

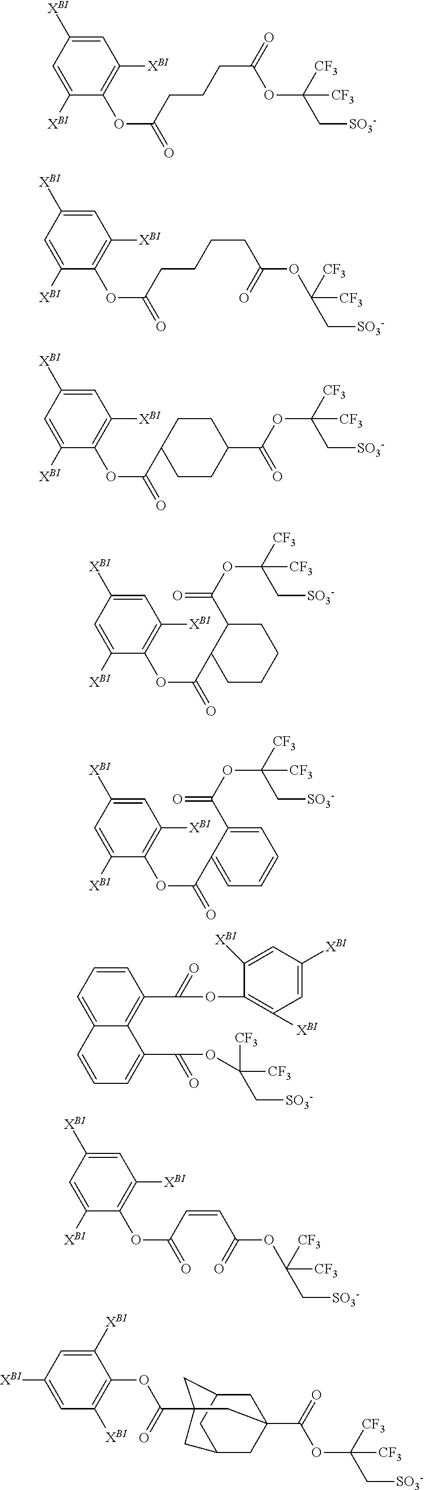

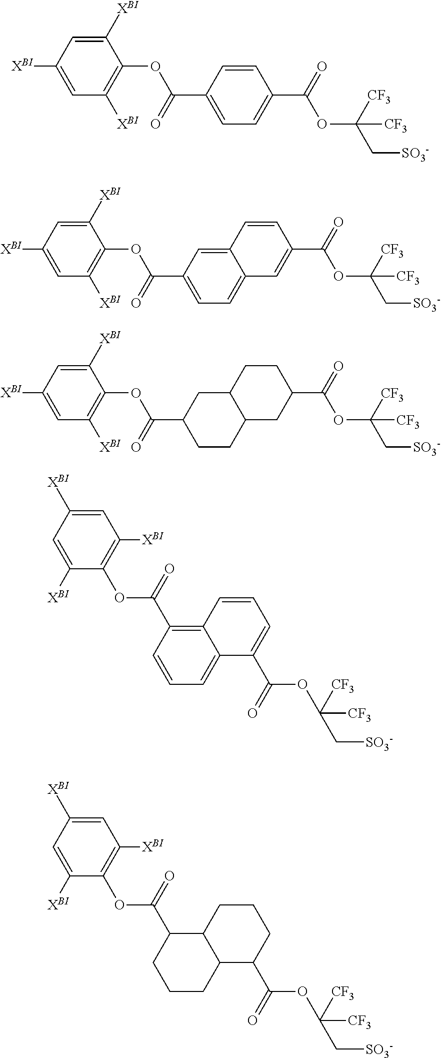

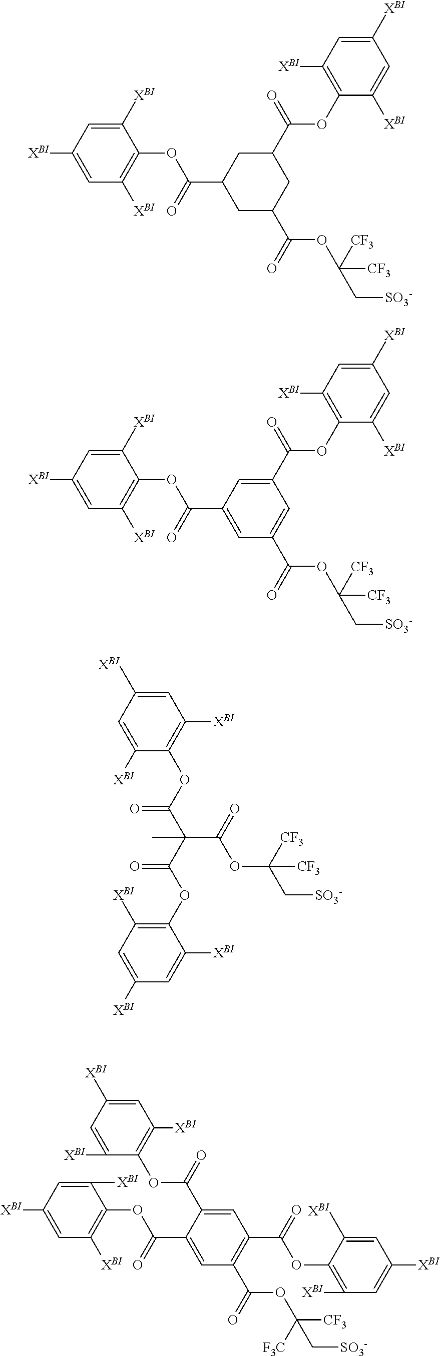

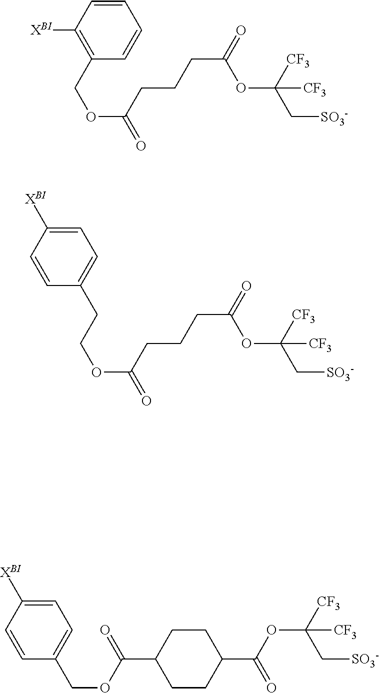

As the non-nucleophilic counterion, a fluorobenzenesulfonic acid anion attached to an aromatic group containing an iodine atom described in JP 6648726, an anion having a mechanism of decomposition by an acid described in WO 2021/200056 A or JP-A 2021-70692, an anion having a cyclic ether group described in JP-A 2018-180525 or JP-A 2021-35935, and an anion described in JP-A 2018-92159 can also be used.

As the non-nucleophilic counterion, an anion of a bulky benzenesulfonic acid derivative free of fluorine atoms described in JP-A 2006-276759, JP-A 2015-117200, JP-A 2016-65016, or JP-A 2019-202974, and a benzenesulfonic acid anion or an alkylsulfonic acid anion free of fluorine atoms attached to an aromatic group containing an iodine atom described in JP 6645464 can also be used.

As the non-nucleophilic counterion, further an anion of a bissulfonic acid described in JP-A 2015-206932, an anion having a sulfonate on one side and a sulfonamide or sulfonimide differing therefrom on the other side described in WO 2020/158366 A, and an anion having a sulfonate on one side and a carboxylate on the other side described in JP-A 2015-24989 can also be used.

The photoacid generator as the component (D) is also preferably a photoacid generator having the formula (5).

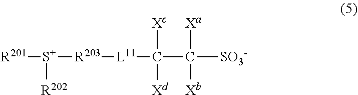

In the formula (5), R²⁰¹ and R²⁰² are each independently a C₁-C₃₀ hydrocarbyl group which may contain a heteroatom. R²⁰³ is a C₁-C₃₀ hydrocarbylene group which may contain a heteroatom, Any two of R²⁰¹, R²⁰², and R²⁰³ may bond together to form a ring with a sulfur atom to which they are attached.

The C₁-C₃₀ hydrocarbyl group of R²⁰¹ or R²⁰² may be saturated or unsaturated and straight, branched, or cyclic. Specific examples thereof include C₁-C₃₀ alkyl groups such as a methyl group, an ethyl group, a n-propyl group, an isopropyl group, a n-butyl group, an isobutyl group, a sec-butyl group, a tert-butyl group, a tert-pentyl group, a n-pentyl group, a n-hexyl group, a n-octyl group, a 2-ethylhexyl group, a n-nonyl group, and a n-decyl group; C₃-C₃₀ cyclic saturated hydrocarbyl groups such as a cyclopentyl group, a cyclohexyl group, a cyclopentylmethyl group, a cyclopentylethyl group, a cyclopentylbutyl group, a cyclohexylmethyl group, a cyclohexylethyl group, a cyclohexylbutyl group, a norbornyl group, an oxanorbornyl group, a tricyclo[5.2.1.0^{2,6}]decyl group, and an adamantyl group; C₆-C₃₀ aryl groups such as a phenyl group, a methylphenyl group, an ethylphenyl group, a n-propylphenyl group, an isopropylphenyl group, a n-butylphenyl group, an isobutylphenyl group, a sec-butylphenyl group, a tert-butylphenyl group, a naphthyl group, a methylnaphthyl group, an ethylnaphthyl group, a n-propylnaphthyl group, an isopropylnaphthyl group, a n-butylnaphthyl group, an isobutylnaphthyl group, a sec-butylnaphthyl group, a tert-butylnaphthyl group, and an anthracenyl group; and a group obtained by combining these groups. Some or all hydrogen atoms in the hydrocarbyl group may be substituted with a group containing a heteroatom such as an oxygen atom, a sulfur atom, a nitrogen atom, or a halogen atom, and some —CH₂— in the hydrocarbyl group may be substituted with a group containing a heteroatom such as an oxygen atom, a sulfur atom, or a nitrogen atom, so that the hydrocarbyl group may contain a hydroxy group, a cyano group, a fluorine atom, a chlorine atom, a bromine atom, an iodine atom, a carbonyl group, an ether bond, an ester bond, a sulfonate ester bond, a carbonate bond, a lactone ring, a sultone ring, a carboxylic anhydride (—C(═O)—O—C(═O)—), a haloalkyl group, or the like.

The C₁-C₃₀ hydrocarbylene group of R²⁰³ may be saturated or unsaturated and straight, branched, or cyclic. Specific examples thereof include C₁-C₃₀ alkanediyl groups such as a methanediyl group, an ethane-1,1-diyl group, an ethane-1,2-diyl group, a propane-1,3-diyl group, a butane-1,4-diyl group, a pentane-1,5-diyl group, a hexane-1,6-diyl group, a heptane-1,7-diyl group, an octane-1,8-diyl group, a nonane-1,9-diyl group, a decane-1,10-diyl group, an undecane-1,11-diyl group, a dodecane-1,12-diyl group, a tridecane-1,13-diyl group, a tetradecane-1,14-diyl group, a pentadecane-1,15-diyl group, a hexadecane-1,16-diyl group, and a heptadecane-1,17-diyl group; C₃-C₃₀ cyclic saturated hydrocarbylene groups such as a cyclopentanediyl group, a cyclohexanediyl group, a norbornanediyl group, and an adamantanediyl group; and arylene groups such as a phenylene group, a methylphenylene group, an ethylphenylene group, a n-propylphenylene group, an isopropylphenylene group, a n-butylphenylene group, an isobutylphenylene group, a sec-butylphenylene group, a tert-butylphenylene group, a naphthylene group, a methylnaphthylene group, an ethylnaphthylene group, a n-propylnaphthylene group, an isopropylnaphthylene group, a n-butylnaphthylene group, an isobutylnaphthylene group, a sec-butylnaphthylene group, and a tert-butylnaphthylene group. Some or all hydrogen atoms in the hydrocarbylene group may be substituted with a group containing a heteroatom such as an oxygen atom, a sulfur atom, a nitrogen atom, or a halogen atom, and some —CH₂-in the hydrocarbylene group may be substituted with a group containing a heteroatom such as an oxygen atom, a sulfur atom, or a nitrogen atom, so that the hydrocarbylene group may contain a hydroxy group, a cyano group, a fluorine atom, a chlorine atom, a bromine atom, an iodine atom, a carbonyl group, an ether bond, an ester bond, a sulfonate ester bond, a carbonate bond, a lactone ring, a sultone ring, a carboxylic anhydride (—C(═O)—O—C(═O)—), a haloalkyl group, or the like. The heteroatom is preferably an oxygen atom.

In the formula (5), L¹¹ is a single bond, an ether bond, or a C₁-C₂₀ hydrocarbylene group which may contain a heteroatom. The hydrocarbylene group may be saturated or unsaturated and straight, branched, or cyclic. Specific examples thereof are as exemplified above for the hydrocarbylene group of R²⁰³.

In the formula (5), X^{a}, X^{b}, X^{c}, and X^{d} are each independently a hydrogen atom, a fluorine atom, or a trifluoromethyl group. However, at least one of X^{a}, X^{b}, X^{c}, and X^{d} is a fluorine atom or a trifluoromethyl group.

As the photoacid generator having the formula (5), a photoacid generator having the formula (5′) is preferred.

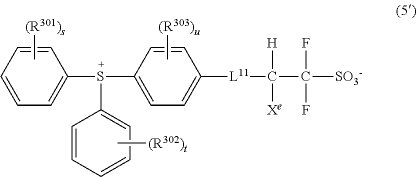

In the formula (5′), L¹¹ is as defined above. X^{e} is a hydrogen atom or a trifluoromethyl group, and preferably a trifluoromethyl group. R³⁰¹, R³⁰², and R³⁰³ are each independently a hydrogen atom or a C₁-C₂₀ hydrocarbyl group which may contain a heteroatom. The hydrocarbyl group may be saturated or unsaturated and straight, branched, or cyclic. Specific examples thereof are as exemplified above for the hydrocarbyl group of R^{fa1} in the formula (Xa-1-1). s and t are each independently 0, 1, 2, 3, 4, or 5, and u is 0, 1, 2, 3, or 4.

Examples of the photoacid generator having the formula (5) are as exemplified above for the photoacid generator having the formula (2) in JP-A 2017-26980.

Among the other photoacid generators, those containing an anion having the formula (Xa-1-1) or (Xa-4) are particularly preferred because of reduced acid diffusion and high solubility in the solvent. In addition, those having the formula (5′) are particularly preferred because of extremely reduced acid diffusion.

When the chemically amplified resist composition of the invention contains the photoacid generator (D), the content thereof is preferably 0.1 to 40 parts by weight, and more preferably 0.5 to 20 parts by weight per 80 parts by weight of the base polymer (A). The amount of the photoacid generator as the component (D) in the above range is preferred because a satisfactory resolution is available without any possibility that a problem of foreign matter occurs after development or during peeling of the resist film. As the another photoacid generator (D), one type may be used alone or two or more types may be combined and used.

### (E) Surfactant

The chemically amplified resist composition of the invention may contain a surfactant as a component (E). The surfactant (E) is preferably a surfactant insoluble or sparingly soluble in water and soluble in an alkaline developer, or a surfactant insoluble or sparingly soluble in water and an alkaline developer. With respect to such a surfactant, those described in JP-A 2010-215608 and JP-A 2011-16746 can be referred to.

As the surfactant insoluble or sparingly soluble in water and an alkaline developer, among the surfactants described in the above-mentioned patent documents, FC-4430 (manufactured by 3M), Surflon® 5-381 (manufactured by AGC Seimi Chemical Co., Ltd.), Olfine® E1004 (manufactured by Nissin Chemical Industry Co., Ltd.), KH-20, KH-30 (manufactured by AGC Seimi Chemical Co., Ltd.), an oxetane ring-opening polymer having the formula (surf-1), and the like are preferred.

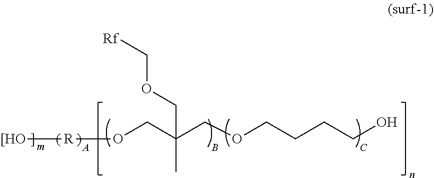

Here, R, Rf, A, B, C, m, n are applied only to the formula (surf-1) regardless of the above description. R is a di- to tetra-valent C₂-C₅ aliphatic group. Examples of the divalent aliphatic group include an ethylene group, a 1,4-butylene group, a 1,2-propylene group, a 2,2-dimethyl-1,3-propylene group, and a 1,5-pentylene group, and examples of the trivalent or tetravalent aliphatic group include the following groups.

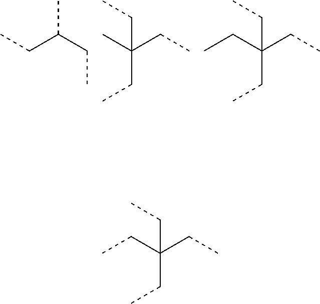

In the formulae, broken lines are points of attachment and are partial structures derived from glycerol, trimethylolethane, trimethylolpropane, and pentaerythritol, respectively.

Among them, a 1,4-butylene group, a 2,2-dimethyl-1,3-propylene group, and the like are preferred.

Rf is a trifluoromethyl group or a pentafluoroethyl group, and preferably a trifluoromethyl group. m is an integer of 0 to 3, n is an integer of 1 to 4, and the sum of n and m is the valence of R and an integer of 2 to 4. A is 1. B is an integer of 2 to 25, and preferably an integer of 4 to 20. C is an integer of 0 to 10, and preferably 0 or 1. The formula (surf-1) does not prescribe the arrangement of respective constituent units while the units may be arranged either blockwise or randomly. The production of a partially fluorinated oxetane ring-opening polymer-based surfactant is described in detail in U.S. Pat. No. 5,650,483 and the like.

When a resist protective film is not used in ArF immersion lithography, a surfactant insoluble or sparingly soluble in water and soluble in an alkaline developer has a function of reducing penetration of water or leaching by orientation on the surface of the resist film. Therefore, such a surfactant is useful for inhibiting elution of a water-soluble component from the resist film to reduce damage to an exposure apparatus, and is also useful because such a surfactant becomes soluble during development with an alkaline aqueous solution after the exposure or after post exposure bake (PEB), and hardly forms foreign matter causing a defect. The preferred surfactant is a polymeric surfactant which is insoluble or sparingly soluble in water and soluble in an alkaline developer, also referred to as “hydrophobic resin”, and especially is water repellent and enhances water sliding.

Specific examples of such a polymeric surfactant include those containing at least one type selected from repeat units having the formulae (6A) to (6E).

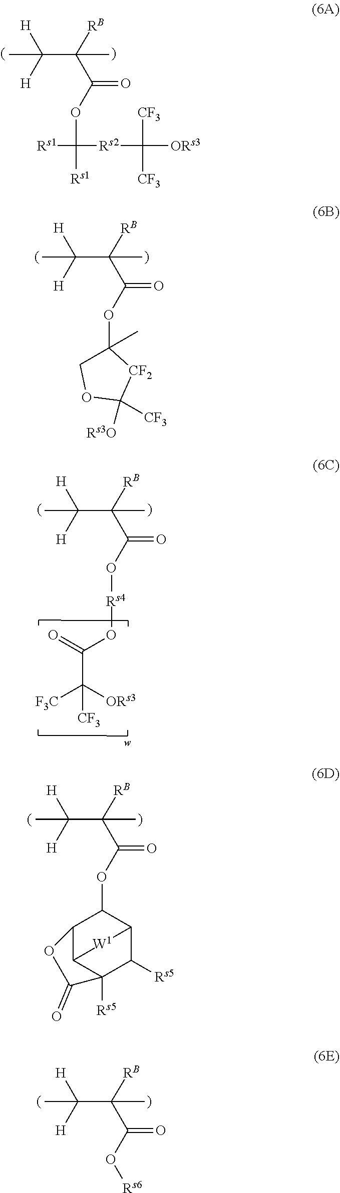

In the formulae (6A) to (6E), R^{B} is a hydrogen atom, a fluorine atom, a methyl group, or a trifluoromethyl group. W¹ is —CH₂—, —CH₂CH₂—, —O—, or two separate —H. R^{s1}'s are each independently a hydrogen atom or a C₁-C₁₀ hydrocarbyl group, R^{s2} is a single bond or a C₁-C₅ straight or branched hydrocarbylene group. R^{s3}'s are each independently a hydrogen atom, a C₁-C₁₅ hydrocarbyl group, a fluorinated hydrocarbyl group, or an acid labile group. When R^{s3} is a hydrocarbyl group or a fluorinated hydrocarbyl group, an ether bond or a carbonyl group may intervene in a carbon-carbon bond. R^{s4} is a C₁-C₂₀ (w+1)-valent hydrocarbon group or fluorinated hydrocarbon group. w is 1, 2, or 3. R^{s5}'s are each independently a hydrogen atom or a group of —C(═O)—O—R^{sa}. R^{sa} is a C₁-C₂₀ fluorinated hydrocarbyl group. R^{s6} is a C₁-C₁₅ hydrocarbyl group or a fluorinated hydrocarbyl group, and an ether bond or a carbonyl group may intervene in a carbon-carbon bond.

The C₁-C₁₀ hydrocarbyl group of R^{s1} is preferably a saturated hydrocarbyl group, and may be straight, branched, or cyclic. Specific examples thereof include C₁-C₁₀ alkyl groups such as a methyl group, an ethyl group, a n-propyl group, an isopropyl group, a n-butyl group, an isobutyl group, a sec-butyl group, a tert-butyl group, a n-pentyl group, a n-hexyl group, a n-heptyl group, a n-octyl group, a n-nonyl group, and a n-decyl group; and C₃-C₁₀ cyclic saturated hydrocarbyl groups such as a cyclopropyl group, a cyclobutyl group, a cyclopentyl group, a cyclohexyl group, an adamantyl group, and a norbornyl group. Among them, C₁-C₆ groups are preferred.

The hydrocarbylene group of R^{s2} is preferably a saturated hydrocarbylene group, and may be straight, branched, or cyclic. Specific examples thereof include a methylene group, an ethylene group, a propylene group, a butylene group, and a pentylene group.

The hydrocarbyl group of R^{s3} or R^{s6} may be saturated or unsaturated and straight, branched, or cyclic. Specific examples thereof include saturated hydrocarbyl groups and aliphatic unsaturated hydrocarbyl groups such as an alkenyl group and an alkynyl group, and saturated hydrocarbyl groups are preferred. Examples of the saturated hydrocarbyl group include an undecyl group, a dodecyl group, a tridecyl group, a tetradecyl group, and a pentadecyl group in addition to those exemplified above for the hydrocarbyl group of R^{s1}. Examples of the fluorinated hydrocarbyl group of R^{s3} or R^{s6} include groups in which some or all hydrogen atoms attached to carbon atoms of the above-mentioned hydrocarbyl group are substituted with fluorine atoms. As described above, an ether bond or a carbonyl group may intervene in a carbon-carbon bond.

Specific examples of the acid labile group of R^{s3} include groups having the formulae (AL-3) to (AL-5), a trialkylsilyl group in which each alkyl group is a C₁-C₆ alkyl group, and C₄-C₂₀ oxoalkyl groups.

The (w+1)-valent hydrocarbon group or fluorinated hydrocarbon group of R^{s4} may be straight, branched, or cyclic, and specific examples thereof include groups obtained by further eliminating “w” number of hydrogen atoms from the above-mentioned hydrocarbyl groups, fluorinated hydrocarbyl groups, and the like.

The fluorinated hydrocarbyl group of R^{sa} is preferably a saturated hydrocarbyl group, and may be straight, branched, or cyclic. Specific examples thereof include those in which some or all hydrogen atoms in the hydrocarbyl group are substituted with fluorine atoms, and specific examples thereof include a trifluoromethyl group, a 2,2,2-trifluoroethyl group, a 3,3,3-trifluoro-1-propyl group, a 3,3,3-trifluoro-2-propyl group, a 2,2,3,3-tetrafluoropropyl group, a 1,1,1,3,3,3-hexafluoroisopropyl group, a 2,2,3,3,4,4,4-heptafluorobutyl group, a 2,2,3,3,4,4,5,5-octafluoropentyl group, a 2,2,3,3,4,4,5,5,6,6,7,7-dodecafluoroheptyl group, a 2-(perfluorobutyl)ethyl group, a 2-(perfluorohexyl)ethyl group, a 2-(perfluorooctyl)ethyl group, and a 2-(perfluorodecyl)ethyl group.

Specific examples of the repeat units having the formulae (6A) to (6E) include those shown below, but are not limited thereto. In the following formulae, R^{B} is as defined above.

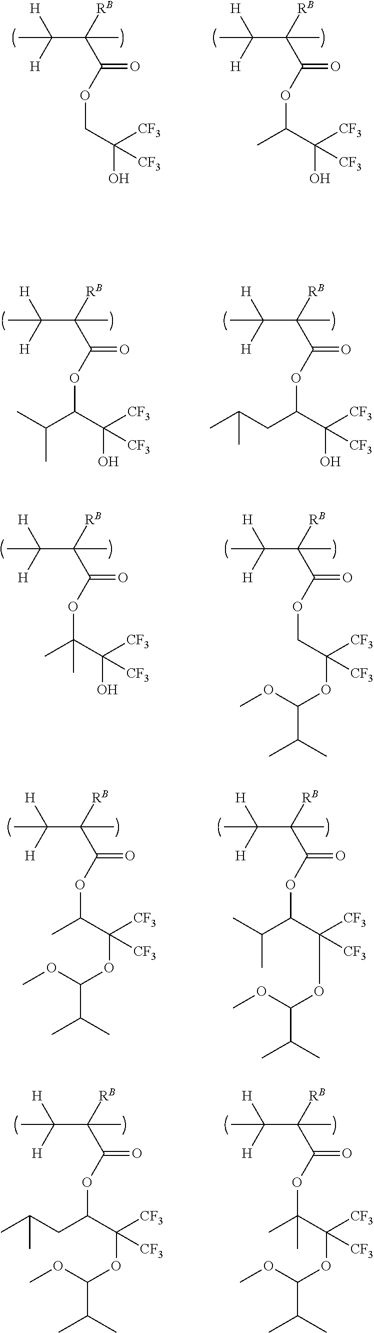

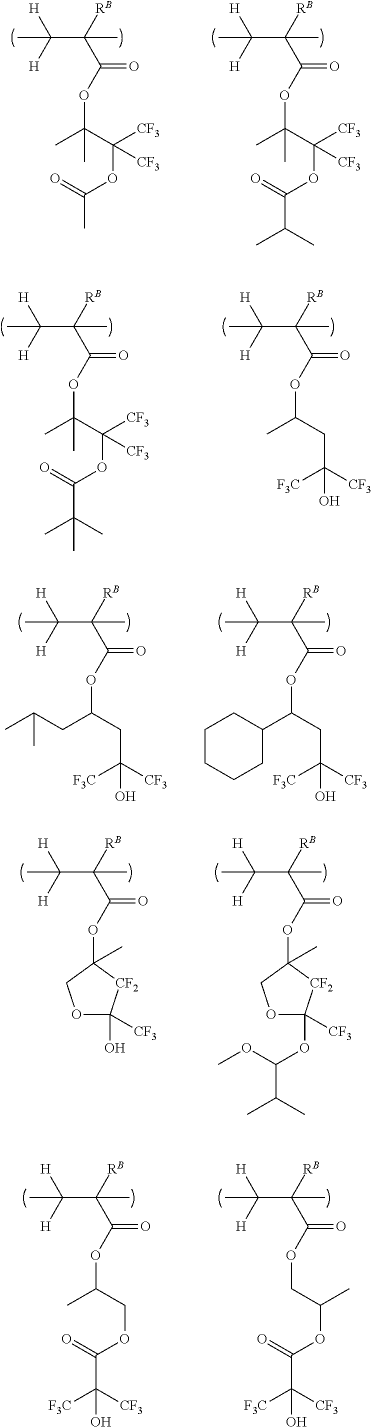

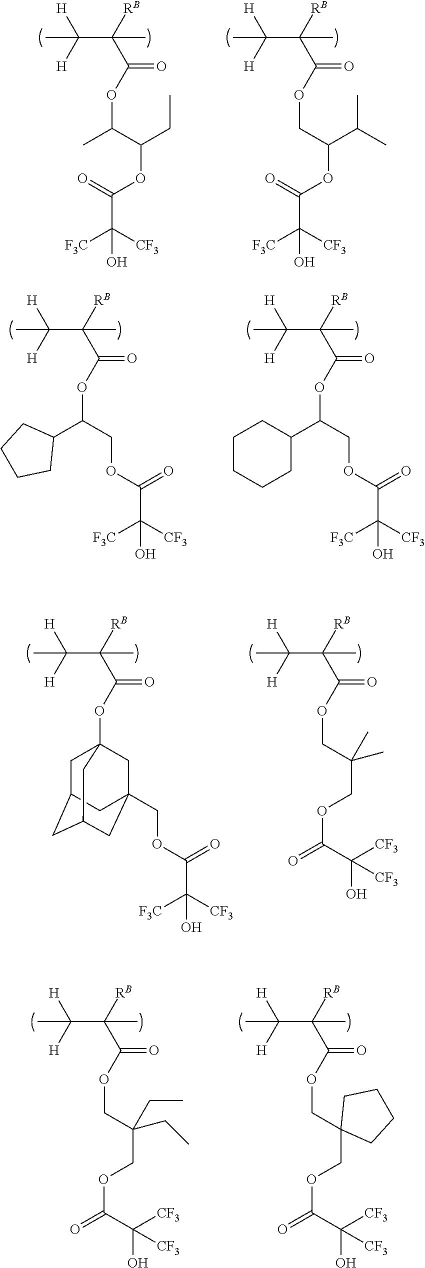

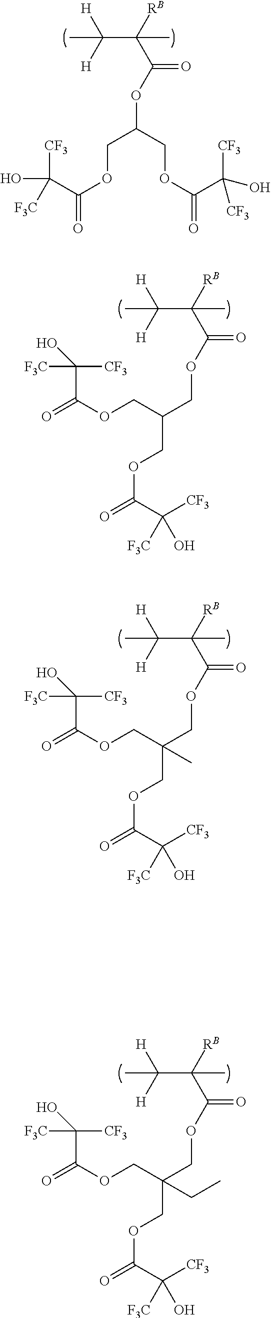

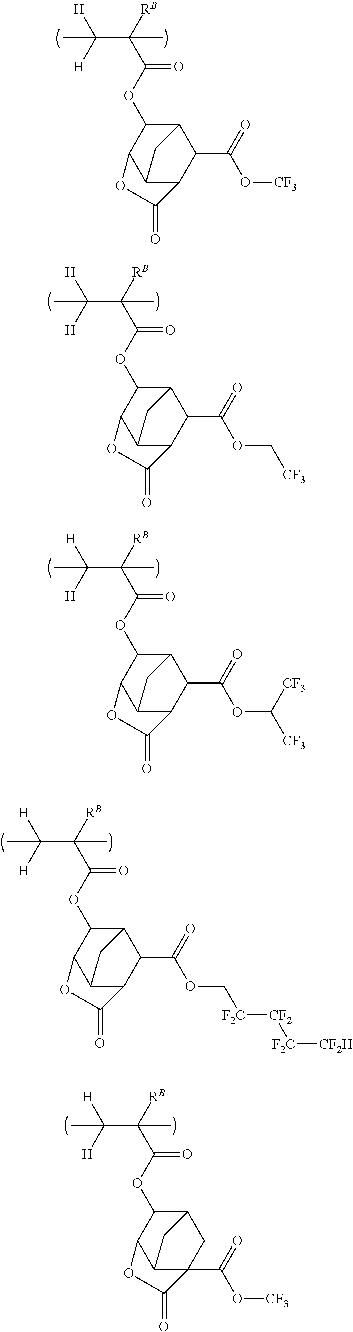

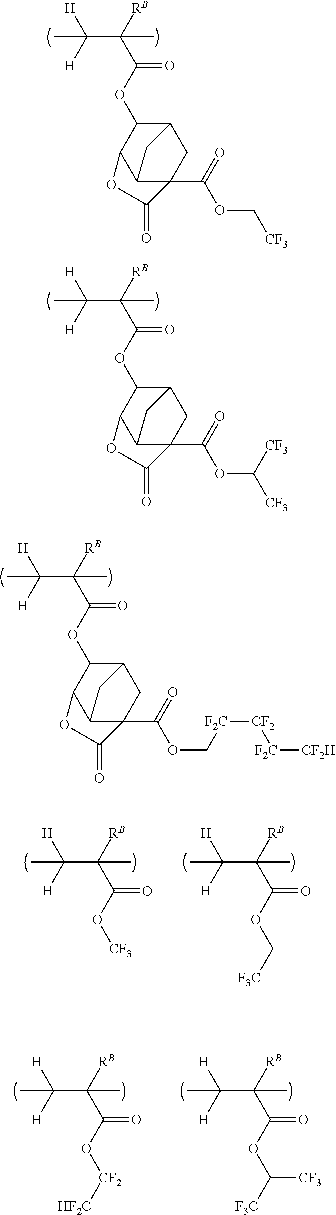

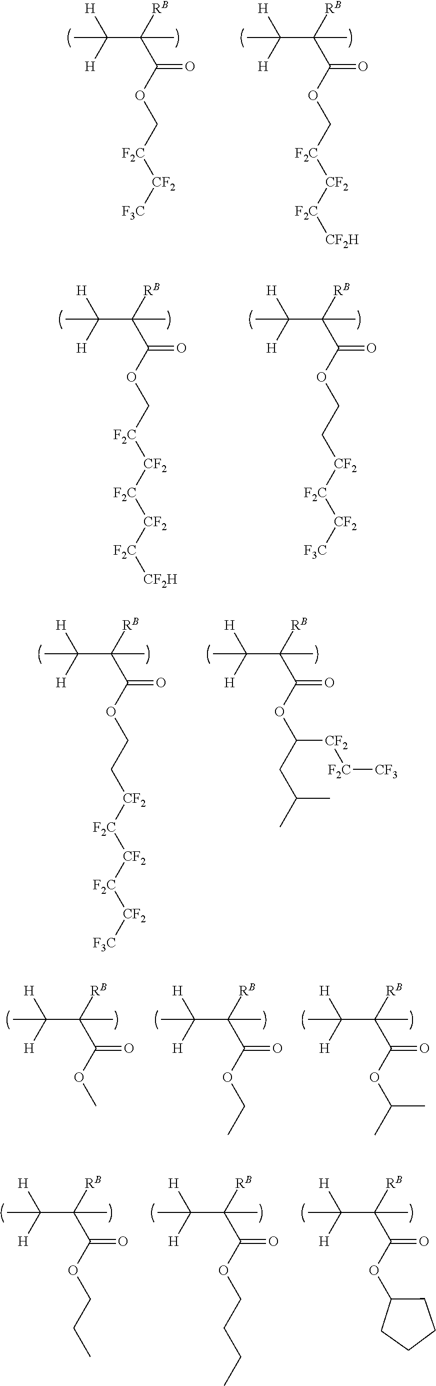

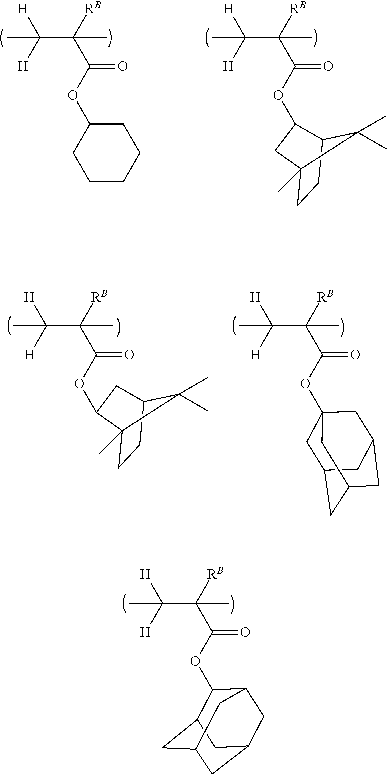

The polymeric surfactant may further contain another repeat unit other than the repeat units having the formulae (6A) to (6E). Examples of the another repeat unit include repeat units obtained from methacrylic acid, α-trifluoromethylacrylic acid derivatives, and the like. The content of the repeat units having the formulae (6A) to (6E) in the polymeric surfactant is preferably 20 mol % or more, more preferably 60 mol % or more, still more preferably 100 mol % of the overall repeat units.

The polymeric surfactant preferably has a Mw of 1,000 to 500,000, and more preferably 3,000 to 100,000. Mw/Mn is preferably 1.0 to 2.0, and more preferably 1.0 to 1.6.

Examples of the method for synthesizing the polymeric surfactant include a method in which a monomer containing a repeat unit having any one of the formulae (6A) to (6E) and, if necessary, an unsaturated bond to give another repeat unit is heated and polymerized by adding a radical initiator in an organic solvent. Examples of the organic solvent used in the polymerization include toluene, benzene, THF, diethyl ether, and dioxane. Specific examples of the polymerization initiator include AIBN, 2,2′-azobis(2,4-dimethylvaleronitrile), dimethyl 2,2-azobis(2-methylpropionate), benzoyl peroxide, and lauroyl peroxide. The reaction temperature is preferably 50 to 100° C. The reaction time is preferably 4 to 24 hours. The acid labile group that has been incorporated in the monomer may be kept as such, or the polymerization may be followed by protection or partial protection.

When the polymeric surfactant is synthesized, a known chain transfer agent such as dodecylmercaptan or 2-mercaptoethanol may be used for the purpose of adjusting the molecular weight. In that case, the amount of such a chain transfer agent added is preferably 0.01 to 10 mol % based on the total number of moles of the monomer to be polymerized.

When the chemically amplified resist composition of the invention contains the surfactant (E), the content thereof is preferably 0.1 to 50 parts by weight, and more preferably 0.5 to 10 parts by weight per 80 parts by weight of the base polymer (A). When the content of the surfactant (E) is 0.1 parts by weight or more, the receding contact angle between the surface of the resist film and water is sufficiently improved, and when the content of the surfactant is 50 parts by weight or less, the dissolution rate of the surface of the resist film in the developer is low, and the height of the formed fine pattern is sufficiently maintained. As the surfactant (E), one type may be used alone or two or more types may be combined and used.

### (F) Other Components

The chemically amplified resist composition of the invention may contain, as another component (F), a compound which is decomposed with an acid to generate another acid (acid amplifier compound), an organic acid derivative, a fluorine-substituted alcohol, a compound having a Mw of 3,000 or less and changes its solubility in a developer by the action of an acid (dissolution inhibitor), or the like. As the acid amplifier compound, compounds described in JP-A 2009-269953 or JP-A 2010-215608 can be referred to. When the acid amplifier compound is contained, the content thereof is preferably 0 to 5 parts by weight, and more preferably 0 to 3 parts by weight per 80 parts by weight of the base polymer (A). If the content is too large, it is difficult to control acid diffusion, and deterioration in resolution and deterioration in pattern shape may occur. As the organic acid derivative, the fluorine-substituted alcohol, and the dissolution inhibitor, compounds described in JP-A 2009-269953 or JP-A 2010-215608 can be referred to.

### Pattern Forming Process

A pattern forming process of the invention includes the steps of forming a resist film on a substrate using the chemically amplified resist composition, exposing the resist film to high-energy radiation, and developing the exposed resist film using a developer.

As the substrate, for example, a substrate for producing an integrated circuit (such as Si, SiO₂, SiN, SiON, TiN, WSi, BPSG, SOG, or an organic antireflective film) or a substrate for producing a mask circuit (such as Cr, CrO, CrON, MoSi₂, or SiO₂) can be used.

The resist film can be formed by, for example, applying the chemically amplified resist composition onto a substrate by a method such as spin coating so as to have a film thickness of preferably 0.05 to 2 μm, and prebaking the chemically amplified resist composition on a hotplate at preferably 60 to 150° C. for 1 to 10 minutes, more preferably at 80 to 140° C. for 1 to 5 minutes.

Examples of the high-energy radiation used for exposure of the resist film include KrF excimer laser light, ArF excimer laser light, EB, and EUV with a wavelength of 3 to nm. When KrF excimer laser light, ArF excimer laser light, or EUV is used, exposure can be performed by using a mask for forming a desired pattern and performing irradiation so that the exposure dose is preferably 1 to 200 mJ/cm² and more preferably 10 to 100 mJ/cm². When EB is used, irradiation is performed using a mask for forming a desired pattern or directly so that the exposure dose is preferably 1 to 300 μC/cm² and more preferably 10 to 200 μC/cm².

The exposure can be performed by an ordinary exposure method, or can be performed by also using an immersion method in which a liquid having a refractive index of 1.0 or more is interposed between the resist film and a projection lens. In this case, a protective film insoluble in water can also be used.

The protective film insoluble in water is used for preventing an eluted material from the resist film and for improving water sliding on the film surface, and roughly includes two types. One is of an organic solvent removal type, in which removal with an organic solvent not dissolving the resist film is required before development with an alkaline aqueous solution, and the other is of an alkaline aqueous solution-soluble type, in which the protective film is soluble in an alkaline developer and removed together with removal of a soluble portion in the resist film. The latter is particularly preferably a material that contains, as a base, a polymer having a 1,1,1,3,3,3-hexafluoro-2-propanol residue insoluble in water and soluble in an alkaline developer, and that is dissolved in an alcohol-based solvent having 4 or more carbon atoms, a C₈-C₁₂ ether-based solvent, or a mixed solvent thereof. The latter can also be a material obtained by dissolving the above-mentioned surfactant insoluble in water and soluble in an alkaline developer in an alcohol-based solvent having 4 or more carbon atoms, a C₈-C₁₂ ether-based solvent, or a mixed solvent thereof.

PEB may be performed after the exposure. PEB can be performed, for example, by heating on a hotplate at preferably 60 to 150° C. for 1 to 5 minutes, more preferably at 80 to 140° C. for 1 to 3 minutes.

In the development, for example, a developer of an alkaline aqueous solution, such as tetramethylammonium hydroxide (TMAH) at preferably 0.1 to 5 wt %, more preferably 2 to 3 wt % is used, and the development is performed for preferably 0.1 to 3 minutes, more preferably 0.5 to 2 minutes by a conventional method such as a dip method, a puddle method, or a spray method to dissolve the exposed portion and form a desired pattern on the substrate.

After the resist film is formed, the acid generator or the like may be extracted from the film surface by performing rinsing with pure water, or particles may be washed off, or rinsing for removing water remaining on the film after exposure may be performed.

Furthermore, a pattern may be formed by a double patterning method. Examples of the double patterning method include a trench method in which an underlayer of a 1:3 trench pattern is processed by first exposure and etching, and a 1:3 trench pattern is formed by second exposure while shifting the position to form a 1:1 pattern, and a line method in which a first underlayer of a 1:3 isolated left pattern is processed by first exposure and etching, and a second underlayer having the formed 1:3 isolated left pattern under the first underlayer is processed by second exposure while shifting the position to form a 1:1 pattern with a half pitch.

In the pattern forming process of the invention, a method of negative tone development in which an unexposed portion is dissolved using an organic solvent as a developer instead of the alkaline aqueous solution may be used.

In the organic solvent development, as the developer, 2-octanone, 2-nonanone, 2-heptanone, 3-heptanone, 4-heptanone, 2-hexanone, 3-hexanone, diisobutyl ketone, methylcyclohexanone, acetophenone, methylacetophenone, propyl acetate, butyl acetate, isobutyl acetate, pentyl acetate, butenyl acetate, isopentyl acetate, propyl formate, butyl formate, isobutyl formate, pentyl formate, isopentyl formate, methyl valerate, methyl pentenoate, methyl crotonate, ethyl crotonate, methyl propionate, ethyl propionate, ethyl 3-ethoxypropionate, methyl lactate, ethyl lactate, propyl lactate, butyl lactate, isobutyl lactate, pentyl lactate, isopentyl lactate, methyl 2-hydroxyisobutyrate, ethyl 2-hydroxyisobutyrate, methyl benzoate, ethyl benzoate, phenyl acetate, benzyl acetate, methyl phenylacetate, ethyl phenylacetate, benzyl formate, phenylethyl formate, methyl 3-phenylpropionate, benzyl propionate, 2-phenylethyl acetate, or the like can be used. One type of these organic solvents may be used alone, or two or more types of organic solvents may be mixed and used.

### EXAMPLES

Hereinafter, the invention is specifically described with showing Synthesis Examples, Examples, and Comparative Examples, but the invention is not limited to the following Examples. The apparatus used is as follows.
MALDI TOF-MS: S3000 manufactured by JEOL Ltd.

### [1] Synthesis of Sulfonium Salt Monomer

### Example 1-1

### Synthesis of PAG-1

### (1) Synthesis of Intermediate in-1

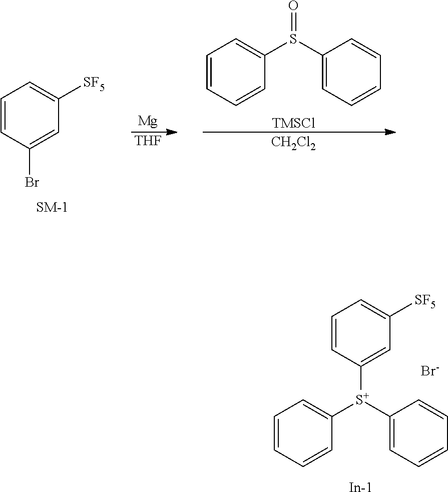

Under a nitrogen atmosphere, a Grignard reagent was prepared from magnesium (4.5 g), THF (100 g) and an intermediate In-1 (51.0 g). The reaction system was cooled to 10° C. or lower, and a solution containing diphenyl sulfoxide (12.1 g) and methylene chloride (50 g) was added thereto. After the addition, chlorotrimethylsilane (19.6 g) was added dropwise while the temperature in the reaction system was maintained at 20° C. or lower. After the dropwise addition, the mixture was aged for 2 hours at a temperature of 20° C. or lower in the reaction system. After aging, the reaction system was cooled, and an aqueous solution containing ammonium chloride (15 g), 20 wt % hydrochloric acid (15 g) and water (100 g) was added dropwise to stop the reaction. Thereafter, methanol (50 g) and diisopropyl ether (200 g) were added thereto, and an aqueous layer was separated. Further, the separated aqueous layer was washed twice with hexane (200 g). Methylene chloride (100 g) was added to the washed aqueous layer to extract a desired product. Thereafter, ordinary aqueous work-up was performed, and the solvent was distilled off to obtain 24.5 g of an intermediate In-2 as a colorless oily product (yield: 87%).

### (2) Synthesis of PAG-1

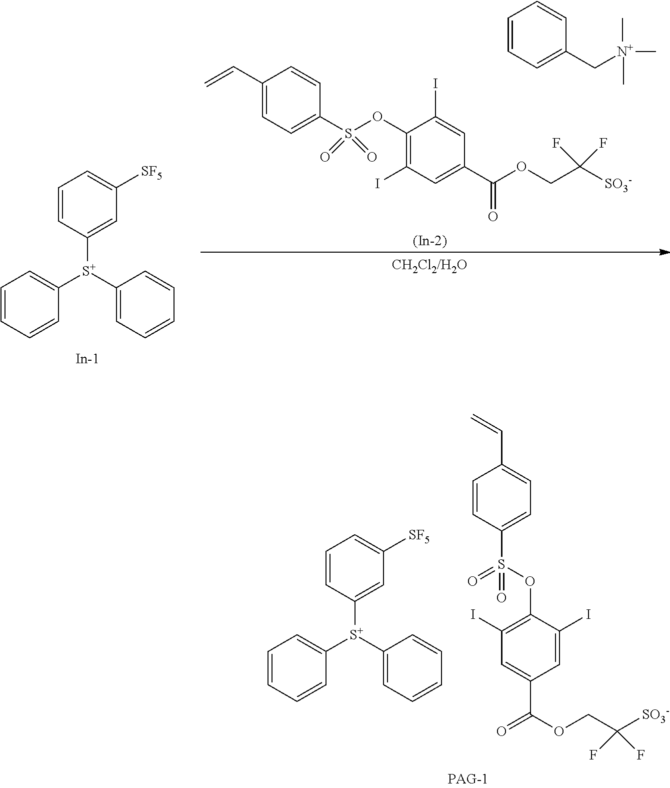

Under a nitrogen atmosphere, the intermediate In-1 (24.5 g), the intermediate In-2 (40.3 g), methylene chloride (150 g), and water (70 g) were added, and the mixture was stirred at room temperature for 30 minutes. The organic layer was separated, washed with water, and then concentrated under reduced pressure. The residue was washed with diisopropyl ether and concentrated to obtain 51.7 g of PAG-1 as an oily product (yield: 91%).

The results of TOF-MS of PAG-1 are shown below.

### MALDI TOF-MS:

POSITIVE M⁺ 389 (corresponding to C₁₈H₁₄F₄S₂⁺)
NEGATIVE M⁻ 699 (corresponding to C₁₇H₁₁F₂I₂O₈S₂⁻)

### Examples 1-2 to 1-9

### Synthesis of PAG-2 to PAG-9

The following sulfonium salt monomers PAG-2 to PAG-9 were synthesized by various organic synthesis reactions using corresponding raw materials.

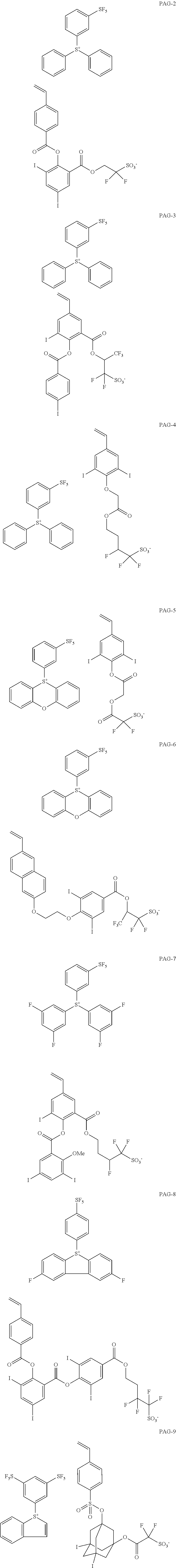

### Comparative Examples 1-1 to 1-6

### Synthesis of PAG-A to PAG-F for Comparison

The following sulfonium salt monomers PAG-A to PAG-F for comparison were synthesized by various organic synthesis reactions using corresponding raw materials.

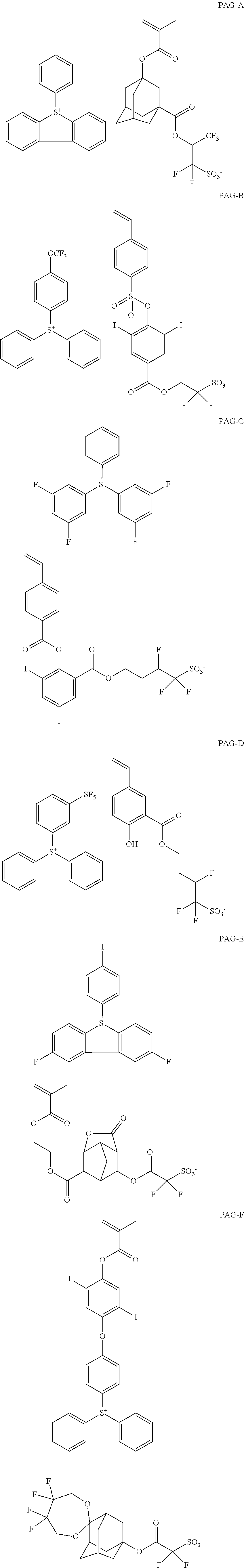

### [2] Synthesis of Base Polymer

Among the monomers used for synthesis of the base polymer, monomers other than PAG-1 to PAG-9 and PAG-A to PAG-F for comparison are as follows.

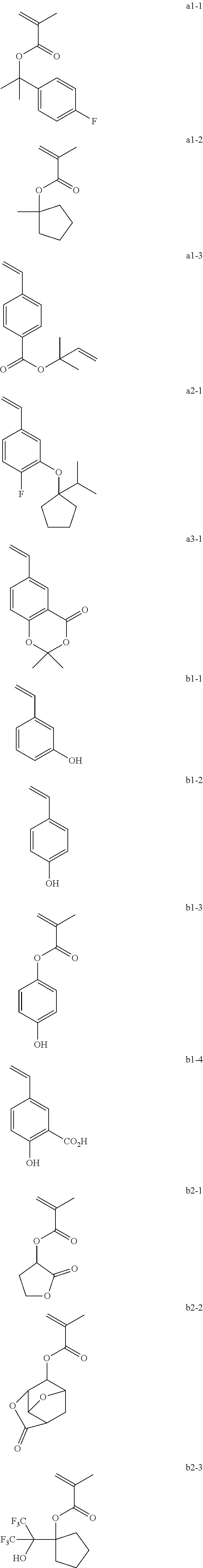

### Example 2-1

### Synthesis of Polymer P-1

Under a nitrogen atmosphere, a flask was charged with a monomer a1-1 (38.2 g), a monomer b1-1 (22.2 g), PAG-1 (50.8 g), V-601 (manufactured by Wako Pure Chemical Industries, Ltd.) (3.58 g), and MEK (127 g) to prepare a monomer-polymerization initiator solution. Another flask under a nitrogen atmosphere was charged with 46 g of MEK, heated to 80° C. with stirring, and then the monomer-polymerization initiator solution was added dropwise thereto over 4 hours. After completion of the dropwise addition, stirring was continued for 2 hours while the temperature of the polymerization liquid was maintained at 80° C., and then the polymerization liquid was cooled to room temperature. The obtained polymerization liquid was added dropwise to 2,000 g of hexane with vigorous stirring, and the precipitated polymer was separated by filtration. Further, the obtained polymer was washed twice with 600 g of hexane, and then vacuum dried at 50° C. for 20 hours to obtain a white powdery polymer P-1 as white powder (amount: 98.1 g, yield: 98%). The polymer P-1 had a Mw of 9,700, and a Mw/Mn of 1.61. Mw is a measurement value by GPC in terms of polystyrene using DMF as a solvent.

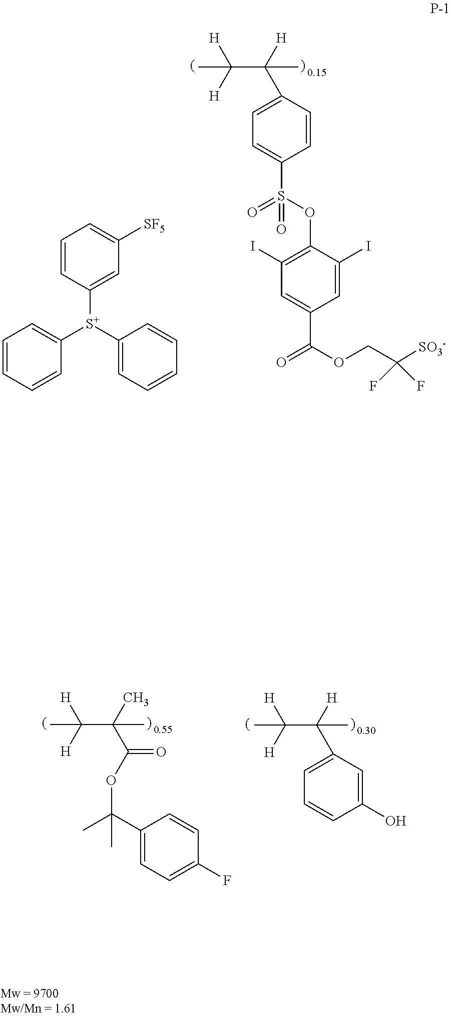

### Examples 2-2 to 2-26, Comparative Examples 2-1 to 2-18

### Synthesis of Polymers P-2 to P-26 and CP-1 to CP-18

Polymers shown in Tables 1 and 2 were produced by the same procedure as in Example 2-1 except that the type and blending ratio of monomers were changed.

TABLE 1

| | | Incorpo- | | Incorpo- | | Incorpo- | | Incorpo- | | Incorpo- | | |
|---|---|---|---|---|---|---|---|---|---|---|---|---|
| | | ration | | ration | | ration | | ration | | ration | | |
| | | ratio | | ratio | | ratio | | ratio | | ratio | | |
| Polymer | Unit 1 | (mol %) | Unit 2 | (mol %) | Unit 3 | (mol %) | Unit 4 | (mol %) | Unit 5 | (mol %) | Mw | Mw/Mn |
| P-1 | PAG-1 | 15 | a1-1 | 55 | b1-1 | 30 | — | — | — | — | 9,700 | 1.61 |
| P-2 | PAG-2 | 15 | a1-1 | 55 | b1-1 | 30 | — | — | — | — | 9,200 | 1.62 |
| P-3 | PAG-3 | 15 | a1-1 | 55 | b1-1 | 30 | — | — | — | — | 9,500 | 1.63 |
| P-4 | PAG-4 | 15 | a1-1 | 55 | b1-1 | 30 | — | — | — | — | 9,400 | 1.63 |
| P-5 | PAG-5 | 15 | a1-1 | 55 | b1-1 | 30 | — | — | — | — | 9,100 | 1.61 |
| P-6 | PAG-6 | 15 | a1-1 | 55 | b1-1 | 30 | — | — | — | — | 9,300 | 1.62 |
| P-7 | PAG-7 | 15 | a1-1 | 55 | b1-1 | 30 | — | — | — | — | 9,200 | 1.65 |
| P-8 | PAG-8 | 15 | a1-1 | 55 | b1-1 | 30 | — | — | — | — | 9,100 | 1.61 |
| P-9 | PAG-9 | 15 | a1-1 | 55 | b1-1 | 30 | — | — | — | — | 9,700 | 1.62 |
| P-10 | PAG-1 | 15 | a1-2 | 55 | b1-1 | 30 | — | — | — | — | 9,400 | 1.60 |
| P-11 | PAG-1 | 15 | a1-3 | 55 | b1-1 | 30 | — | — | — | — | 9,100 | 1.61 |
| P-12 | PAG-1 | 15 | a2-1 | 55 | b1-1 | 30 | — | — | — | — | 9,800 | 1.62 |
| P-13 | PAG-1 | 15 | a3-1 | 45 | b1-1 | 40 | — | — | — | — | 8,900 | 1.63 |
| P-14 | PAG-2 | 15 | a1-1 | 55 | b1-2 | 30 | — | — | — | — | 9,300 | 1.61 |
| P-15 | PAG-2 | 15 | a1-1 | 55 | b1-3 | 30 | — | — | — | — | 9,000 | 1.62 |
| P-16 | PAG-2 | 15 | a1-1 | 55 | b1-4 | 30 | — | — | — | — | 9,300 | 1.64 |
| P-17 | PAG-1 | 15 | a1-1 | 30 | a2-1 | 20 | b1-1 | 35 | — | — | 9,200 | 1.62 |
| P-18 | PAG-3 | 15 | a1-1 | 35 | a3-1 | 15 | b1-2 | 35 | — | — | 9,900 | 1.61 |
| P-19 | PAG-4 | 15 | a1-2 | 30 | a2-1 | 15 | b1-3 | 40 | — | — | 9,500 | 1.62 |
| P-20 | PAG-6 | 10 | a1-1 | 35 | a2-1 | 15 | b1-1 | 30 | b2-1 | 10 | 9,400 | 1.63 |
| P-21 | PAG-7 | 15 | a1-2 | 35 | a3-1 | 15 | b1-2 | 25 | b2-2 | 10 | 9,200 | 1.64 |
| P-22 | PAG-9 | 15 | a1-1 | 50 | b1-1 | 30 | b2-3 | 5 | — | — | 8,900 | 1.61 |
| P-23 | PAG-1 | 5 | a1-1 | 55 | b1-2 | 40 | — | — | — | — | 9,700 | 1.62 |
| P-24 | PAG-2 | 5 | a1-1 | 30 | a1-3 | 25 | b1-2 | 40 | — | — | 9,400 | 1.63 |
| P-25 | PAG-3 | 5 | a1-2 | 30 | a2-1 | 20 | b1-4 | 35 | b2-1 | 10 | 9,200 | 1.61 |
| P-26 | PAG-7 | 5 | a1-1 | 35 | a3-1 | 15 | b1-1 | 30 | b2-2 | 15 | 9,400 | 1.62 |

TABLE 2

| | | Incorpo- | | Incorpo- | | Incorpo- | | Incorpo- | | Incorpo- | | |
|---|---|---|---|---|---|---|---|---|---|---|---|---|
| | | ration | | ration | | ration | | ration | | ration | | |
| | | ratio | | ratio | | ratio | | ratio | | ratio | | |
| Polymer | Unit 1 | (mol %) | Unit 2 | (mol %) | Unit 3 | (mol %) | Unit 4 | (mol %) | Unit 5 | (mol %) | Mw | Mw/Mn |
| CP-1 | PAG-A | 15 | a1-1 | 55 | b1-1 | 30 | — | — | — | — | 9,500 | 1.62 |
| CP-2 | PAG-B | 15 | a1-1 | 55 | b1-1 | 30 | — | — | — | — | 9,100 | 1.61 |
| CP-3 | PAG-C | 15 | a1-1 | 55 | b1-1 | 30 | — | — | — | — | 9,300 | 1.63 |
| CP-4 | PAG-D | 15 | a1-1 | 55 | b1-1 | 30 | — | — | — | — | 9,100 | 1.62 |
| CP-5 | PAG-E | 15 | a1-1 | 55 | b1-1 | 30 | — | — | — | — | 9,000 | 1.65 |
| CP-6 | PAG-F | 15 | a1-1 | 55 | b1-1 | 30 | — | — | — | — | 9,500 | 1.62 |
| CP-7 | PAG-B | 15 | a1-2 | 55 | b1-1 | 30 | — | — | — | — | 9,600 | 1.61 |
| CP-8 | PAG-C | 15 | a3-1 | 45 | b1-1 | 40 | — | — | — | — | 9,700 | 1.63 |
| CP-9 | PAG-D | 15 | a1-1 | 55 | b1-3 | 30 | — | — | — | — | 9,500 | 1.63 |
| CP-10 | PAG-E | 15 | a1-1 | 55 | b1-4 | 30 | — | — | — | — | 9,400 | 1.62 |
| CP-11 | PAG-B | 15 | a1-1 | 35 | a3-1 | 15 | b1-2 | 35 | — | — | 9,700 | 1.64 |
| CP-12 | PAG-D | 10 | a1-1 | 35 | a2-1 | 15 | b1-1 | 30 | b2-1 | 10 | 9,500 | 1.62 |
| CP-13 | PAG-C | 15 | a1-2 | 35 | a3-1 | 15 | b1-2 | 25 | b2-2 | 10 | 9,300 | 1.61 |
| CP-14 | PAG-F | 15 | a1-1 | 50 | b1-1 | 30 | b2-3 | 5 | — | — | 9,100 | 1.60 |
| CP-15 | PAG-A | 5 | a1-1 | 55 | b1-2 | 40 | — | — | — | — | 9,300 | 1.62 |
| CP-16 | PAG-D | 5 | a1-2 | 30 | a2-1 | 20 | b1-4 | 35 | b2-1 | 10 | 9,400 | 1.63 |
| CP-17 | a1-1 | 60 | b1-1 | 40 | — | — | — | — | — | — | 5,700 | 1.55 |
| CP-18 | a1-1 | 50 | b1-2 | 30 | b2-1 | 20 | — | — | — | — | 6,100 | 1.54 |

### [13] Preparation of Chemically Amplified Resist Composition

### Examples 3-1 to 3-26 and Comparative Examples 3-1 to 3-18

Chemically amplified resist compositions (R-1 to R-26, CR-1 to CR-18) were prepared by dissolving a base polymer (P-1 to P-26) containing the sulfonium salt monomer (PAG-1 to PAG-9) of the invention or a base polymer (CP-1 to CP-18) containing a sulfonium salt monomer for comparison (PAG-A to PAG-F), another photoacid generator (PAG-X or PAG-Y), and a quencher (Q-1 to Q-4) in a solvent containing 0.01 wt % of surfactant A (OMNOVA Solutions, Inc.) to prepare a solution in accordance with the formulation shown in Tables 3 and 4, and filtering the solution through a Teflon® filter with a pore size of 0.2 μm.

TABLE 3

| | | | | | Photoacid | | | |
|---|---|---|---|---|---|---|---|---|
| | | Resist | Base polymer | Quencher | generator | Solvent 1 | Solvent 2 | Solvent 3 |
| | | composition | (pbw) | (pbw) | (pbw) | (pbw) | (pbw) | (pbw) |
| Example | 3-1 | R-1 | P-1 (80) | Q-1 (8.0) | — | PGMEA (2250) | EL (2800) | DAA (550) |
| | 3-2 | R-2 | P-2 (80) | Q-1 (8.2) | — | PGMEA (2250) | EL (2800) | DAA (550) |
| | 3-3 | R-3 | P-3 (80) | Q-1 (8.0) | — | PGMEA (2250) | EL (2800) | DAA (550) |
| | 3-4 | R-4 | P-4 (80) | Q-1 (7.8) | — | PGMEA (2250) | EL (2800) | DAA (550) |
| | 3-5 | R-5 | P-5 (80) | Q-1 (8.0) | — | PGMEA (2250) | EL (2800) | DAA (550) |
| | 3-6 | R-6 | P-6 (80) | Q-1 (8.0) | — | PGMEA (2250) | EL (2800) | DAA (550) |
| | 3-7 | R-7 | P-7 (80) | Q-1 (8.2) | — | PGMEA (2250) | EL (2800) | DAA (550) |
| | 3-8 | R-8 | P-8 (80) | Q-1 (8.0) | — | PGMEA (2250) | EL (2800) | DAA (550) |
| | 3-9 | R-9 | P-9 (80) | Q-1 (7.6) | — | PGMEA (2250) | EL (2800) | DAA (550) |
| | 3-10 | R-10 | P-10 (80) | Q-2 (8.2) | — | PGMEA (2250) | EL (2800) | DAA (550) |
| | 3-11 | R-11 | P-11 (80) | Q-3 (8.0) | — | PGMEA (2250) | EL (2800) | DAA (550) |
| | 3-12 | R-12 | P-12 (80) | Q-1 (8.0) | — | PGMEA (2250) | EL (2800) | DAA (550) |
| | 3-13 | R-13 | P-13 (80) | Q-1 (8.2) | — | PGMEA (2250) | EL (2800) | DAA (550) |
| | 3-14 | R-14 | P-14 (80) | Q-1 (8.0) | — | PGMEA (2250) | EL (2800) | DAA (550) |
| | 3-15 | R-15 | P-15 (80) | Q-3 (7.8) | — | PGMEA (2250) | EL (2800) | DAA (550) |
| | 3-16 | R-16 | P-16 (80) | Q-2 (8.0) | — | PGMEA (2250) | EL (2800) | DAA (550) |
| | 3-17 | R-17 | P-17 (80) | Q-1 (8.0) | — | PGMEA (2250) | EL (2800) | DAA (550) |
| | 3-18 | R-18 | P-18 (80) | Q-1 (7.8) | — | PGMEA (2250) | EL (2800) | DAA (550) |
| | 3-19 | R-19 | P-19 (80) | Q-2 (8.0) | — | PGMEA (2250) | EL (2800) | DAA (550) |
| | 3-20 | R-20 | P-20 (80) | Q-3 (7.8) | PAG-Y (15) | PGMEA (2250) | EL (2800) | DAA (550) |
| | 3-21 | R-21 | P-21 (80) | Q-1 (4.0) | — | PGMEA (2250) | EL (2800) | DAA (550) |
| | | | | Q-4 (3.8) | | | | |
| | 3-22 | R-22 | P-22 (80) | Q-1 (8.2) | — | PGMEA (2250) | EL (2800) | DAA (550) |
| | 3-23 | R-23 | P-23 (80) | Q-3 (7.6) | — | PGMEA (2250) | EL (2800) | DAA (550) |
| | 3-24 | R-24 | P-24 (80) | Q-1 (8.0) | PAG-X (10) | PGMEA (2250) | EL (2800) | DAA (550) |
| | 3-25 | R-25 | P-25 (80) | Q-2 (8.2) | PAG-X (10) | PGMEA (2250) | EL (2800) | DAA (550) |
| | 3-26 | R-26 | P-26 (80) | Q-3 (8.0) | PAG-Y (15) | PGMEA (2250) | EL (2800) | DAA (550) |

TABLE 4

| | | | | | Photoacid | | | |
|---|---|---|---|---|---|---|---|---|
| | | Resist | Base polymer | Quencher | generator | Solvent 1 | Solvent 2 | Solvent 3 |
| | | composition | (pbw) | (pbw) | (pbw) | (pbw) | (pbw) | (pbw) |
| Comparative | 3-1 | CR-1 | CP-1 (80) | Q-1 (8.0) | — | PGMEA (2250) | EL (2800) | DAA (550) |
| Example | 3-2 | CR-2 | CP-2 (80) | Q-1 (8.2) | — | PGMEA (2250) | EL (2800) | DAA (550) |
| | 3-3 | CR-3 | CP-3 (80) | Q-1 (8.0) | — | PGMEA (2250) | EL (2800) | DAA (550) |
| | 3-4 | CR-4 | CP-4 (80) | Q-1 (7.8) | — | PGMEA (2250) | EL (2800) | DAA (550) |
| | 3-5 | CR-5 | CP-5 (80) | Q-1 (8.0) | — | PGMEA (2250) | EL (2800) | DAA (550) |
| | 3-6 | CR-6 | CP-6 (80) | Q-1 (8.2) | — | PGMEA (2250) | EL (2800) | DAA (550) |
| | 3-7 | CR-7 | CP-7 (80) | Q-2 (8.2) | — | PGMEA (2250) | EL (2800) | DAA (550) |
| | 3-8 | CR-8 | CP-8 (80) | Q-1 (8.2) | — | PGMEA (2250) | EL (2800) | DAA (550) |
| | 3-9 | CR-9 | CP-9 (80) | Q-3 (7.8) | — | PGMEA (2250) | EL (2800) | DAA (550) |
| | 3-10 | CR-10 | CP-10 (80) | Q-2 (8.0) | — | PGMEA (2250) | EL (2800) | DAA (550) |
| | 3-11 | CR-11 | CP-11 (80) | Q-1 (8.0) | — | PGMEA (2250) | EL (2800) | DAA (550) |
| | 3-12 | CR-12 | CP-12 (80) | Q-3 (7.8) | PAG-Y (15) | PGMEA (2250) | EL (2800) | DAA (550) |
| | 3-13 | CR-13 | CP-13 (80) | Q-1 (4.0) | — | PGMEA (2250) | EL (2800) | DAA (550) |
| | | | | Q-4 (3.8) | | | | |
| | 3-14 | CR-14 | CP-14 (80) | Q-1 (8.2) | — | PGMEA (2250) | EL (2800) | DAA (550) |
| | 3-15 | CR-15 | CP-15 (80) | Q-3 (7.6) | — | PGMEA (2250) | EL (2800) | DAA (550) |
| | 3-16 | CR-16 | CP-16 (80) | Q-2 (8.2) | PAG-X (10) | PGMEA (2250) | EL (2800) | DAA (550) |
| | 3-17 | CR-17 | CP-17 (80) | Q-1 (8.0) | PAG-X (24) | PGMEA (2250) | EL (2800) | DAA (550) |
| | 3-18 | CR-18 | CP-18 (80) | Q-1 (8.0) | PAG-Y (24) | PGMEA (2250) | EL (2800) | DAA (550) |

In Tables 1 and 2, the solvents, other photoacid generators PAG-X and PAG-Y, quenchers Q-1 to Q-4, and surfactant A are as follows.

### Solvent:

PGMEA (propylene glycol monomethyl ether acetate)
EL (ethyl lactate)
DAA (diacetone alcohol)
   Another photoacid generators: PAG-X or PAG-Y

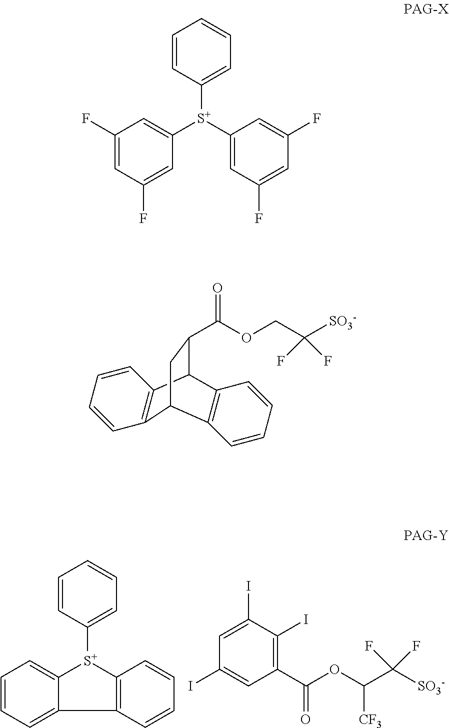

### Quenchers: Q-1 to Q-4

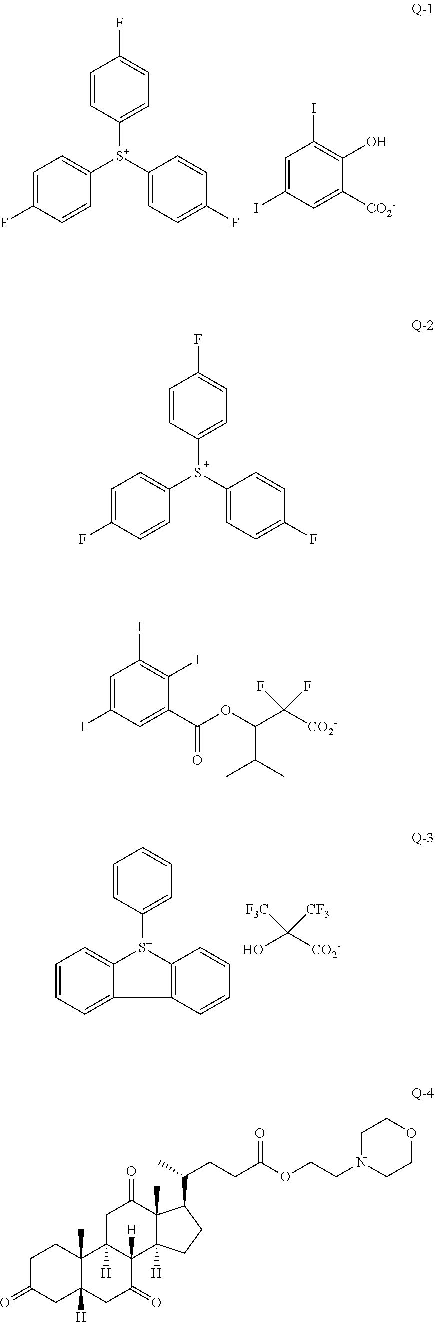

### Surfactant A:

3-methyl-3-(2,2,2-trifluoroethoxymethyl)oxetane/tetrahydrofuran/
2,2-dimethyl-1,3-propanediol copolymer (manufactured by OMNOVA Solutions, Inc.)

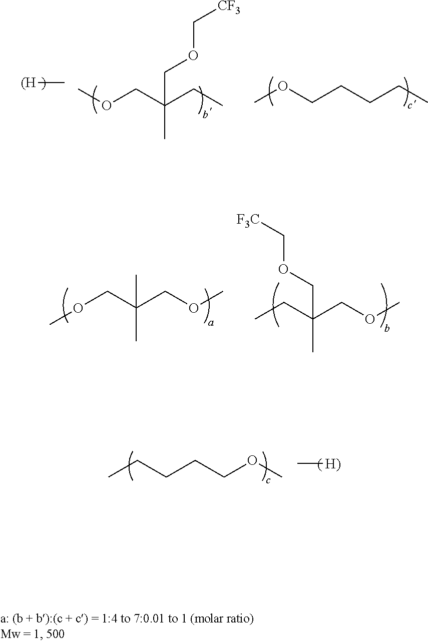

### [4] EUV Lithography Evaluation (1)

Examples 4-1 to 4-26 and Comparative Examples 4-1 to 4-18 Each of the chemically amplified resist compositions (R-1 to R-26, CR-1 to CR-18) shown in Tables 3 and 4 was applied by spin coating onto a Si substrate having a silicon-containing spin-on-hard mask SHB-A940 manufactured by Shin-Etsu Chemical Co., Ltd. (silicon content: 43 wt %) formed to a film thickness of 20 nm and prebaked on a hotplate at 100° C. for 60 seconds to prepare a resist film having a film thickness of 50 nm. The resist film was exposed using an EUV scanner NXE3400 (NA 0.33, a 0.9/0.6, dipole illumination) manufactured by ASML Holding N.V. through a mask bearing an LS pattern having a width of 18 nm and a pitch of 36 nm on-wafer size while changing the exposure dose and focus (exposure dose pitch: 1 mJ/cm², focus pitch: 0.020 μm), and after the exposure, PEB was performed at a temperature shown in Tables 5 and 6 for 60 seconds. This was followed by puddle development in a 2.38 wt % TMAH aqueous solution for seconds, rinsing with a surfactant-containing rinse material, and spin drying to obtain a positive pattern.

The obtained LS pattern was observed with a length measuring SEM (CG6300) manufactured by Hitachi High-Technologies Corporation, and the sensitivity, EL, LWR, depth of focus (DOF), and collapse limit were evaluated according to the following methods. The results are shown in Tables 5 and 6.

### [Evaluation of Sensitivity]

The optimum exposure dose Eop (mJ/cm²) which provided an LS pattern with a line width of 18 nm and a pitch of 36 nm was determined and reported as sensitivity. A smaller value indicates a higher sensitivity.

### [Evaluation of EL]

EL (unit:%) was determined from the exposure dose which provides the LS pattern with a space width of 18 nm±10% (16.2 to 19.8 nm) according to the following formula. A larger value indicates better performance.

$$EL ( \% ) = ( \vert {E}_{1} - {E}_{2} \vert / Eop ) \times 100$$

E₁: an optimum exposure dose which provides an LS pattern with a line width of 16.2 nm and a pitch of 36 nm
E₂: an optimum exposure dose which provides an LS pattern with a line width of 19.8 nm and a pitch of 36 nm
Eop: an optimum exposure dose which provides an LS pattern with a line width of 18 nm and a pitch of 36 nm

### [Evaluation of LWR]

In the LS pattern formed by exposure at Eop, the line width was measured at 10 longitudinally spaced apart points, from which a 3-fold value (3σ) of the standard deviation (σ) was determined as LWR. A smaller value indicates that a pattern having small roughness and a uniform line width is obtained.

### [Evaluation of DOF]

As an index of the depth of focus, a range of focus which provided an LS pattern with a size of 18 nm±10% (16.2 to 19.8 nm) was determined. A greater value indicates a wider depth of focus.

### [Evaluation of Collapse Limit of Line Pattern]

The line size of the LS pattern at each exposure dose with the optimum focus was measured at 10 longitudinally spaced apart points. A narrowest line size obtained without collapse was determined as a collapse limit size. A smaller value indicates better collapse limit.

TABLE 5

| | | | | Optimum | | | | |
|---|---|---|---|---|---|---|---|---|
| | | | PEB | exposure | | | | Collapse |
| | | Resist | temp. | dose | EL | LWR | DOF | limit |
| | | composition | (° C.) | (mJ/cm²) | (%) | (nm) | (nm) | (nm) |
| Example | 4-1 | R-1 | 95 | 32 | 18 | 2.3 | 120 | 10.8 |
| | 4-2 | R-2 | 100 | 33 | 19 | 2.4 | 110 | 10.7 |
| | 4-3 | R-3 | 100 | 32 | 17 | 2.5 | 120 | 10.6 |
| | 4-4 | R-4 | 95 | 32 | 18 | 2.3 | 110 | 11.1 |
| | 4-5 | R-5 | 105 | 33 | 17 | 2.4 | 100 | 11.2 |
| | 4-6 | R-6 | 100 | 35 | 17 | 2.5 | 120 | 11.3 |
| | 4-7 | R-7 | 95 | 34 | 18 | 2.5 | 110 | 11.2 |
| | 4-8 | R-8 | 95 | 32 | 19 | 2.3 | 100 | 11.3 |
| | 4-9 | R-9 | 100 | 33 | 17 | 2.4 | 110 | 11.2 |
| | 4-10 | R-10 | 100 | 34 | 18 | 2.3 | 120 | 11.3 |
| | 4-11 | R-11 | 100 | 32 | 19 | 2.3 | 120 | 11.4 |
| | 4-12 | R-12 | 95 | 34 | 17 | 2.4 | 110 | 10.8 |
| | 4-13 | R-13 | 105 | 33 | 18 | 2.5 | 120 | 10.6 |
| | 4-14 | R-14 | 100 | 32 | 17 | 2.3 | 100 | 10.9 |
| | 4-15 | R-15 | 95 | 33 | 17 | 2.4 | 110 | 10.7 |
| | 4-16 | R-16 | 95 | 33 | 18 | 2.4 | 120 | 11.1 |
| | 4-17 | R-17 | 100 | 32 | 17 | 2.5 | 110 | 11.2 |
| | 4-18 | R-18 | 95 | 34 | 18 | 2.3 | 110 | 11.4 |
| | 4-19 | R-19 | 95 | 33 | 18 | 2.4 | 120 | 11.3 |
| | 4-20 | R-20 | 100 | 34 | 17 | 2.5 | 110 | 10.9 |
| | 4-21 | R-21 | 100 | 35 | 19 | 2.6 | 110 | 10.7 |
| | 4-22 | R-22 | 100 | 32 | 17 | 2.3 | 110 | 11.2 |
| | 4-23 | R-23 | 95 | 33 | 18 | 2.4 | 110 | 11.3 |
| | 4-24 | R-24 | 95 | 32 | 18 | 2.3 | 120 | 11.2 |
| | 4-25 | R-25 | 100 | 34 | 17 | 2.5 | 110 | 11.0 |
| | 4-26 | R-26 | 100 | 32 | 19 | 2.3 | 110 | 10.7 |

TABLE 6

| | | | | Optimum | | | | |
|---|---|---|---|---|---|---|---|---|
| | | | PEB | exposure | | | | Collapse |
| | | Resist | temp. | dose | EL | LWR | DOF | limit |
| | | composition | (° C.) | (mJ/cm²) | (%) | (nm) | (nm) | (nm) |
| Comparative | 4-1 | CR-1 | 95 | 42 | 13 | 3.1 | 90 | 12.8 |
| Example | 4-2 | CR-2 | 100 | 38 | 13 | 2.8 | 80 | 12.7 |
| | 4-3 | CR-3 | 100 | 37 | 14 | 2.7 | 80 | 13.1 |
| | 4-4 | CR-4 | 100 | 39 | 13 | 2.9 | 90 | 12.7 |
| | 4-5 | CR-5 | 95 | 38 | 13 | 2.8 | 80 | 12.3 |
| | 4-6 | CR-6 | 100 | 35 | 11 | 3.7 | 60 | 12.9 |
| | 4-7 | CR-7 | 100 | 37 | 14 | 2.8 | 90 | 11.9 |
| | 4-8 | CR-8 | 100 | 38 | 13 | 2.9 | 80 | 12.1 |
| | 4-9 | CR-9 | 95 | 38 | 14 | 2.8 | 90 | 12.2 |
| | 4-10 | CR-10 | 100 | 38 | 14 | 2.8 | 80 | 12.4 |
| | 4-11 | CR-11 | 100 | 37 | 14 | 2.9 | 80 | 12.1 |
| | 4-12 | CR-12 | 100 | 38 | 14 | 2.9 | 80 | 11.8 |
| | 4-13 | CR-13 | 95 | 38 | 13 | 2.8 | 90 | 11.9 |
| | 4-14 | CR-14 | 105 | 36 | 11 | 3.6 | 50 | 12.9 |
| | 4-15 | CR-15 | 100 | 41 | 13 | 3.2 | 70 | 12.5 |
| | 4-16 | CR-16 | 95 | 37 | 13 | 2.9 | 80 | 12.3 |
| | 4-17 | CR-17 | 95 | 40 | 10 | 3.4 | 60 | 12.6 |
| | 4-18 | CR-18 | 100 | 41 | 10 | 3.3 | 60 | 12.4 |

From the results shown in Tables 5 and 6, it was found that the chemically amplified resist composition containing the polymer formed of the sulfonium salt monomer of the invention has good sensitivity and is excellent in EL, LWR, and DOF. In addition, it was verified that the value of the collapse limit is small and the pattern is resistant to collapse even in fine pattern formation. Therefore, it was shown that the chemically amplified resist composition of the invention is suitable as a material for EUV lithography.

### [5] EUV Lithography Evaluation (2)

### Examples 5-1 to 5-26 and Comparative Examples 5-1 to 5-18

Each of the chemically amplified resist compositions ((R-1 to R-26, CR-1 to CR-18) shown in Tables 3 and 4 was applied by spin coating onto a Si substrate having a silicon-containing spin-on-hard mask SHB-A940 manufactured by Shin-Etsu Chemical Co., Ltd. (silicon content: 43 wt %) formed to a film thickness of 20 nm and prebaked on a hotplate at 105° C. for 60 seconds to prepare a resist film having a film thickness of 50 nm. This resist film was exposed using an EUV scanner NXE3400 (NA 0.33, a 0.9/0.6, quadrupole illumination, a mask bearing a hole pattern having a pitch of 46 nm+20% bias on-wafer size) manufactured by ASML Holding N.V., and PEB was performed at a temperature shown in Tables 7 and 8 for 60 seconds using a hotplate, and development was performed with a 2.38 wt % aqueous TMAH solution for 30 seconds to form a hole pattern with a size of 23 nm.

An exposure dose when a hole with a size of 23 nm was formed was measured using a length measuring SEM (CG6300) manufactured by Hitachi High-Technologies Corporation to determine the exposure dose as a sensitivity, and the sizes of 50 holes at this time were measured, and a 3-fold value (36) of the standard deviation (σ) calculated from the results was determined as CDU. The results are shown in Tables 7 and 8.

TABLE 7

| | | | | Optimum | |
|---|---|---|---|---|---|
| | | Resist | PEB temp. | exposure dose | CDU |
| | | composition | (° C.) | (mJ/cm²) | (nm) |
| Example | 5-1 | R-1 | 95 | 22 | 2.2 |
| | 5-2 | R-2 | 95 | 23 | 2.5 |
| | 5-3 | R-3 | 90 | 23 | 2.2 |
| | 5-4 | R-4 | 90 | 23 | 2.3 |
| | 5-5 | R-5 | 90 | 22 | 2.4 |
| | 5-6 | R-6 | 95 | 24 | 2.5 |
| | 5-7 | R-7 | 95 | 23 | 2.4 |
| | 5-8 | R-8 | 90 | 24 | 2.5 |
| | 5-9 | R-9 | 95 | 24 | 2.4 |
| | 5-10 | R-10 | 95 | 23 | 2.2 |
| | 5-11 | R-11 | 95 | 25 | 2.4 |
| | 5-12 | R-12 | 90 | 22 | 2.6 |
| | 5-13 | R-13 | 90 | 24 | 2.4 |
| | 5-14 | R-14 | 90 | 23 | 2.3 |
| | 5-15 | R-15 | 90 | 23 | 2.4 |
| | 5-16 | R-16 | 85 | 24 | 2.5 |
| | 5-17 | R-17 | 95 | 25 | 2.4 |
| | 5-18 | R-18 | 95 | 24 | 2.2 |
| | 5-19 | R-19 | 90 | 22 | 2.5 |
| | 5-20 | R-20 | 95 | 23 | 2.4 |
| | 5-21 | R-21 | 95 | 23 | 2.4 |
| | 5-22 | R-22 | 95 | 24 | 2.2 |
| | 5-23 | R-23 | 95 | 24 | 2.4 |
| | 5-24 | R-24 | 90 | 25 | 2.5 |
| | 5-25 | R-25 | 95 | 22 | 2.4 |
| | 5-26 | R-26 | 95 | 23 | 2.2 |

TABLE 8

| | | | | Optimum | |
|---|---|---|---|---|---|
| | | Resist | PEB temp. | exposure dose | CDU |
| | | composition | (° C.) | (mJ/cm²) | (nm) |
| Comparative | 5-1 | CR-1 | 95 | 31 | 3.1 |
| Example | 5-2 | CR-2 | 95 | 28 | 2.8 |
| | 5-3 | CR-3 | 95 | 27 | 2.7 |
| | 5-4 | CR-4 | 90 | 27 | 2.8 |
| | 5-5 | CR-5 | 90 | 28 | 2.8 |
| | 5-6 | CR-6 | 95 | 25 | 3.4 |
| | 5-7 | CR-7 | 90 | 28 | 2.8 |
| | 5-8 | CR-8 | 90 | 27 | 2.7 |
| | 5-9 | CR-9 | 90 | 26 | 2.8 |
| | 5-10 | CR-10 | 95 | 28 | 2.8 |
| | 5-11 | CR-11 | 95 | 28 | 2.7 |
| | 5-12 | CR-12 | 95 | 29 | 2.9 |
| | 5-13 | CR-13 | 85 | 27 | 2.8 |
| | 5-14 | CR-14 | 95 | 25 | 3.4 |
| | 5-15 | CR-15 | 95 | 31 | 3.2 |
| | 5-16 | CR-16 | 90 | 29 | 2.9 |
| | 5-17 | CR-17 | 95 | 32 | 3.3 |
| | 5-18 | CR-18 | 95 | 32 | 3.2 |

From the results shown in Tables 7 and 8, it was verified that the chemically amplified resist composition containing the polymer formed of the sulfonium salt monomer of the invention has good sensitivity and is excellent in CDU.

### [6] Evaluation of Dry Etching Resistance

### Examples 6-1 to 6-26 and Comparative Examples 6-1 to 6-18

A polymer solution obtained by dissolving 2 g of each of the polymers (polymers P-1 to P-26, comparative polymers CP-1 to CP-18) shown in Tables 1 and 2 in 10 g of cyclohexanone and filtering the solution through a filter having a pore size of 0.2 μm was applied by spin coating onto a Si substrate to form a film having a thickness of 300 nm, and evaluation was performed under the following conditions.

Etching test with CHF₃/CF₄ gas:

The difference in polymer film thickness before and after etching was determined using a dry etching apparatus TE-8500P manufactured by Tokyo Electron Limited.

Etching conditions are as follows.

| | | |
|---|---|---|
| Chamber pressure | 40 | Pa |
| RF power | 1000 | W |
| Gap | 9 | mm |
| CHF₃ gas flow rate | 30 | mL/min |
| CF₄ gas flow rate | 30 | mL/min |
| Ar gas flow rate | 100 | mL/min |
| Time | 60 | sec |

In this evaluation, a smaller value of film thickness difference, that is, a lower etching rate indicates better etch resistance.

The results of the dry etch resistance are shown in Tables 9 and 10.

TABLE 9

| | | Polymer | CHF₃/CF₄ gas etching rate (nm/min) |
|---|---|---|---|
| Example | 6-1 | P-1 | 96 |
| | 6-2 | P-2 | 97 |
| | 6-3 | P-3 | 96 |
| | 6-4 | P-4 | 95 |
| | 6-5 | P-5 | 96 |
| | 6-6 | P-6 | 96 |
| | 6-7 | P-7 | 97 |
| | 6-8 | P-8 | 95 |
| | 6-9 | P-9 | 96 |
| | 6-10 | P-10 | 97 |
| | 6-11 | P-11 | 96 |
| | 6-12 | P-12 | 97 |
| | 6-13 | P-13 | 96 |
| | 6-14 | P-14 | 98 |
| | 6-15 | P-15 | 97 |
| | 6-16 | P-16 | 95 |
| | 6-17 | P-17 | 98 |
| | 6-18 | P-18 | 97 |
| | 6-19 | P-19 | 96 |
| | 6-20 | P-20 | 98 |
| | 6-21 | P-21 | 96 |
| | 6-22 | P-22 | 97 |
| | 6-23 | P-23 | 96 |
| | 6-24 | P-24 | 98 |
| | 6-25 | P-25 | 95 |
| | 6-26 | P-26 | 97 |

TABLE 10

| | | Polymer | CHF₃/CF₄ gas etching rate (nm/min) |
|---|---|---|---|
| Comparative | 6-1 | CP-1 | 114 |
| Example | 6-2 | CP-2 | 98 |
| | 6-3 | CP-3 | 97 |
| | 6-4 | CP-4 | 98 |
| | 6-5 | CP-5 | 109 |
| | 6-6 | CP-6 | 110 |
| | 6-7 | CP-7 | 98 |
| | 6-8 | CP-8 | 97 |
| | 6-9 | CP-9 | 99 |
| | 6-10 | CP-10 | 105 |
| | 6-11 | CP-11 | 98 |
| | 6-12 | CP-12 | 99 |
| | 6-13 | CP-13 | 97 |
| | 6-14 | CP-14 | 110 |
| | 6-15 | CP-15 | 108 |
| | 6-16 | CP-16 | 99 |
| | 6-17 | CP-17 | 109 |
| | 6-18 | CP-18 | 108 |

From the results shown in Tables 9 and 10, it was verified that the polymer of the invention has excellent dry etch resistance to a CHF₃/CF₄ gas.

Japanese Patent Application No. 2024-087965 is incorporated herein by reference.

Although some preferred embodiments have been described, many modifications and variations may be made thereto in light of the above teachings. It is therefore to be understood that the invention may be practiced otherwise than as specifically described without departing from the scope of the appended claims.

1. A sulfonium salt monomer, having the formula (A): 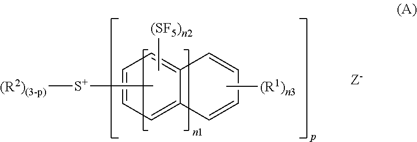
wherein p is 1, 2, or 3, n1 is 0 or 1, n2 is 1 or 2, n3 is 0, 1, 2, or 3, provided that when n1 is 0, 1≤n2+n3≤5, and when n1 is 1, 1≤n2+n3≤7,
R¹ is a halogen atom, a nitro group, a cyano group, a hydroxy group, a C₁-C₂₀ hydrocarbyl group which may contain a heteroatom, a C₁-C₂₀ hydrocarbyloxy group which may contain a heteroatom, or a C₁-C₂₀ hydrocarbylthio group which may contain a heteroatom, when n3 is 2 or 3, a plurality of R¹'s may be identical or different, and two R¹¹'s may bond together to form a ring with a carbon atom to which they are attached,
R² is a halogen atom or a C₁-C₃₀ hydrocarbyl group which may contain a heteroatom, when p is 1, two R²'s may be identical or different, and two of three substituents attached to S⁺ may bond together to form a ring with a sulfur atom to which they are attached, and
Z⁻ is a fluoroalkanesulfonic acid anion having an aromatic vinyl structure and an iodine atom.

2. The sulfonium salt monomer of claim 1, having the formula (A1): 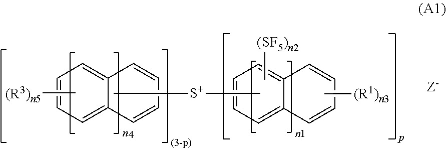
wherein p, n1 to n3, R¹, and Z⁻ are as defined above,
n4 is 0 or 1, n5 is 0, 1, 2, 3, 4, or 5,
R³ is a halogen atom, a nitro group, a cyano group, a hydroxy group, a C₁-C₂₀ hydrocarbyl group which may contain a heteroatom, a C₁-C₂₀ hydrocarbyloxy group which may contain a heteroatom, or a C₁-C₂₀ hydrocarbylthio group which may contain a heteroatom, when n5 is 2 to 5, a plurality of R³'s may be identical or different, and two R³'s may bond together to form a ring with a carbon atom to which they are attached.

3. The sulfonium salt monomer of claim 1, wherein Z⁻ is an anion having the formula (Z): 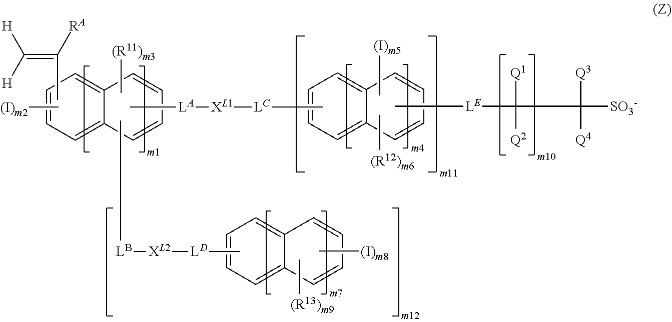
wherein m1 is 0 or 1, m2 is 0, 1, 2, 3, or 4, m3 is 0, 1, 2, or 3, m4 is 0 or 1, m5 is 0, 1, 2, 3, or 4, m6 is 0, 1, 2, or 3, m7 is 0 or 1, m8 is 1,2,3, or 4, m9 is 0, 1, 2, or 3, m10 is 0, 1, 2, 3, or 4, m11 is 0 or 1, m12 is 0 or 1, provided that when m1 is 0, 0≤m2+m3+m12≤4, when m1 is 1, 0≤m2+m3+m12≤6, when m4 is 0, 0≤m5+m6≤4, when m4 is 1, 0≤m5+m6≤6, when m7 is 0, 0≤m8+m9≤5, when m7 is 1, 0≤m8+m9≤7, and 1≤m2+m5+m8≤4,
R^{A}'s are each independently a hydrogen atom, a fluorine atom, a methyl group, or a trifluoromethyl group,
R¹¹, R¹², and R¹³ are each independently a halogen atom other than an iodine atom, a nitro group, a hydroxy group, a C₁-C₂₀ hydrocarbyl group which may contain a heteroatom, a C₁-C₂₀ hydrocarbyloxy group which may contain a heteroatom, or a C₁-C₂₀ hydrocarbylthio group which may contain a heteroatom, when m3 is 2 or 3, a plurality of R¹³'s may be identical or different, and two R¹¹'s may bond together to form a ring with a carbon atom to which they are attached, when m6 is 2 or 3, a plurality of R¹²'s may be identical or different, and two R¹²'s may bond together to form a ring with a carbon atom to which they are attached, when m9 is 2 or 3, a plurality of R¹³'s may be identical or different, and two R¹³'s may bond together to form a ring with a carbon atom to which they are attached,
L^{A}, L^{B}, L^{C}, L^{D}, and L^{E} are each independently a single bond, an ether bond, an ester bond, a sulfonate ester bond, an amide bond, a sulfonamide bond, a carbonate bond, or a carbamate bond,
X^{L1} and X^{L2} are each independently a single bond or a C₁-C₄₀ hydrocarbylene group which may contain a heteroatom,
Q¹ and Q² are each independently a hydrogen atom, a fluorine atom, or a C₁-C₆ fluorinated saturated hydrocarbyl group,
Q³ and Q⁴ are each independently a fluorine atom or a C₁-C₆ fluorinated saturated hydrocarbyl group,
excluding that m11 and m12 are 0 at the same time, and that L^{A}, L^{B}, L^{C}, L^{D}, and X^{L1} and X^{L2} are a single bond at the same time.

4. A monomeric photoacid generator comprising the sulfonium salt monomer of claim 1.

5. A polymer comprising a repeat unit derived from the monomeric photoacid generator of claim 4.

6. The polymer of claim 5, further comprising a repeat unit having the formula (a1) or (a2): 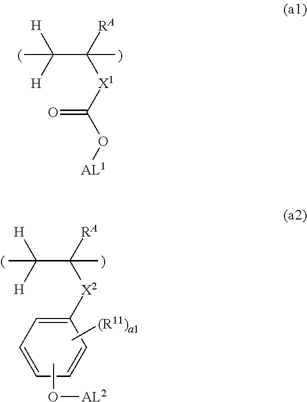
wherein R^{A} s are each independently a hydrogen atom, a fluorine atom, a methyl group, or a trifluoromethyl group,
X¹ is a single bond, a phenylene group, a naphthylene group, or *—C(═O)—O—X¹¹—, and the phenylene group or the naphthylene group may be substituted with a hydroxy group, a nitro group, a cyano group, a C₁-C₁₀ saturated hydrocarbyl group which may contain a fluorine atom, a C₁-C₁₀ saturated hydrocarbyloxy group which may contain a fluorine atom, or a halogen atom, X¹¹ is a C₁-C₁₀ saturated hydrocarbylene group, a phenylene group, or a naphthylene group, and the saturated hydrocarbylene group may contain a hydroxy group, an ether bond, an ester bond, or a lactone ring,
X² is a single bond or *—C(═O)—O—,
* designates a point of attachment to a carbon atom in the backbone,
R²¹ is a halogen atom, a cyano group, a hydroxy group, a nitro group, a C₁-C₂₀ hydrocarbyl group which may contain a heteroatom, a C₁-C₂₀ hydrocarbyloxy group which may contain a heteroatom, a C₂-C₂₀ hydrocarbylcarbonyl group which may contain a heteroatom, a C₂-C₂₀ hydrocarbylcarbonyloxy group which may contain a heteroatom, or a C₂-C₂₀ hydrocarbyloxycarbonyl group which may contain a heteroatom, when a1 is 2 or more, a plurality of R²¹'s may be identical or different,
AL¹ and AL² are each independently an acid labile group, and
a1 is 0, 1, 2, 3, or 4.

7. The polymer of claim 5, further comprising a repeat unit having the formula (a3): 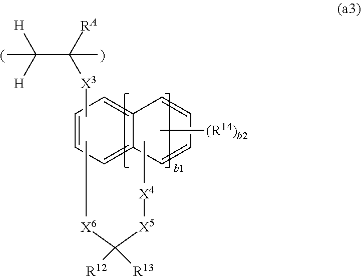
wherein b1 is 0 or 1, b2 is 0, 1, 2, or 3 when b1 is 0, and is 0, 1, 2, 3, 4, or 5 when b1 is 1,
R^{A} is a hydrogen atom, a fluorine atom, a methyl group, or a trifluoromethyl group,
X³ is a single bond, *—C(═O)—O—, or *—C(═O)—NH—, * designates a point of attachment to a carbon atom in the backbone,
R²² and R²³ are each independently a hydrogen atom or a C₁-C₂₀ hydrocarbyl group which may contain a heteroatom, and R²² and R²³ may bond together to form a ring with a carbon atom to which they are attached,
R²⁴ is a halogen atom, a hydroxy group, a cyano group, a nitro group, a C₁-C₂₀ hydrocarbyl group which may contain a heteroatom, a C₁-C₂₀ hydrocarbyloxy group which may contain a heteroatom, a C₂-C₂₀ hydrocarbyloxycarbonyl group which may contain a heteroatom, a C₁-C₂₀ hydrocarbylthio group which may contain a heteroatom, or —N(R^{24A})(R^{24B}), R^{24A} and R^{24B} are each independently a hydrogen atom or a C₁-C₆ hydrocarbyl group, when b2 is 2 or more, a plurality of R²⁴'s may be identical or different, and a plurality of R²⁴'s may bond together to form a ring with a carbon atom on an aromatic ring to which they are attached,
X⁴ is a single bond, a C₁-C₄ aliphatic hydrocarbylene group, a carbonyl group, a sulfonyl group, or a group obtained by combining these groups, and
X⁵ and X⁶ are each independently an oxygen atom or a sulfur atom, provided that X⁴ and X⁶ are attached to an adjacent carbon atom on an aromatic ring.

8. The polymer of claim 5, further comprising a repeat unit having the formula (b1) or (b2): 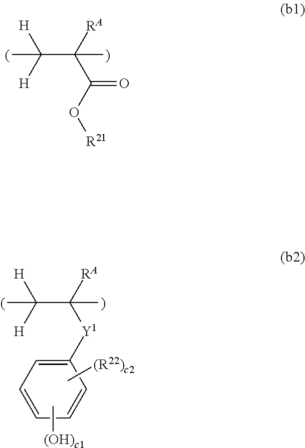
wherein R^{A}'s are each independently a hydrogen atom, a fluorine atom, a methyl group, or a trifluoromethyl group,
Y¹ is a single bond or *—C(═O)—O—, * designates a point of attachment to a carbon atom in the backbone,
R³¹ is a hydrogen atom or a C₁-C₂₀ group containing at least one or more structures selected from a hydroxy group other than a phenolic hydroxy group, a cyano group, a carbonyl group, a carboxy group, an ether bond, an ester bond, a sulfonate ester bond, a carbonate bond, a lactone ring, a sultone ring, and a carboxylic anhydride (—C(═O)—O—C(═O)—),
R³² is a halogen atom, a hydroxy group, a nitro group, a C₁-C₂₀ hydrocarbyl group which may contain a heteroatom, a C₁-C₂₀ hydrocarbyloxy group which may contain a heteroatom, a C₂-C₂₀ hydrocarbylcarbonyl group which may contain a heteroatom, a C₂-C₂₀ hydrocarbylcarbonyloxy group which may contain a heteroatom, or a C₂-C₂₀ hydrocarbyloxycarbonyl group which may contain a heteroatom, when c2 is 2 or more, a plurality of R³²'s may be identical or different,
c1 is an integer of 1 to 4, and c2 is an integer of 0 to 4, provided that 1≤b+c≤5.

9. A chemically amplified resist composition comprising (A) a base polymer containing the polymer of claim 5.

10. The chemically amplified resist composition of claim 9, further comprising (B) an organic solvent.

11. The chemically amplified resist composition of claim 9, further comprising (C) a quencher.

12. The chemically amplified resist composition of claim 9, further comprising (D) a photoacid generator.

13. The chemically amplified resist composition of claim 9, further comprising (E) a surfactant.

14. A pattern forming process comprising the steps of forming a resist film on a substrate using the chemically amplified resist composition of claim 9, exposing the resist film to high-energy radiation, and developing the exposed resist film using a developer.

15. The pattern forming process of claim 14, wherein the high-energy radiation is KrF excimer laser light, ArF excimer laser light, an electron beam, or an extreme ultraviolet ray with a wavelength of 3 to 15 nm.
